(19) United States
(12) Patent Application Publication
(10) Pub. No.: US 2006/0063760 A1
(43) Pub. Date: Mar. 23, 2006

(54) Hydroxamic acids useful in the treatment of hyper-proliferative disorders

(75) Inventors: Yamin Wang, Sandy Hook, CT (US); Maio Dai, Briarwood, NY (US); Holia Hatoum-Mokdad, Hamden, CT (US); Zhengqiu Hong, Milford, CT (US); Harold C. E. Kluender, Trumbull, CT (US); Gaetan H. Ladouceur, Guilford, CT (US); Tindy Li, West Haven, CT (US); Derek B. Lowe, Hamden, CT (US); Eric S. Mull, Guilford, CT (US); Tatiana E. Shelekhin, Fidgefield, CT (US); Roger A. Smith, Madison, CT (US); Wai C. Wong, Trumbull, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: BAYER PHARMACEUTICALS CORPORATION, WEST HAVEN, CT (US)

(21) Appl. No.: 10/554,390

(22) PCT Filed: Apr. 16, 2004
(86) PCT No.: PCT/US04/11990

§ 371 Date: Oct. 20, 2005

Related U.S. Application Data
(60) Provisional application No. 60/463,479, filed on Apr. 17, 2003.
(60) Provisional application No. 60/484,053, filed on Jun. 30, 2003.

(51) Int. Cl. A61K 31/54; A61K 31/537; A61K 31/445; A61K 31/495; A61K 31/4172

(52) U.S. Cl. 514/227.5; 514/310; 514/237.5; 514/252.12; 514/319; 514/575; 544/59; 544/159; 544/398; 546/205; 562/621; 514/400

(57) ABSTRACT

This invention relates to a compound of Formula (I) and its use in treating hyper-proliferative disorders.

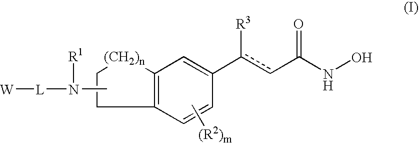

### FIELD OF THE INVENTION

This invention relates to novel hydroxamic acid compounds, pro-drugs thereof, pharmaceutical compositions containing such compounds and pro-drugs, and the use of those compounds or compositions for treating hyper-proliferative disorders.

### COMPOUNDS OF THE INVENTION

One embodiment of the present invention is a compound of Formula I 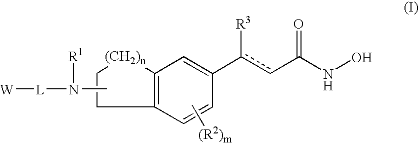
wherein
W is selected from H, (C₁-C₆)alkyl,
   O-phenyl optionally substituted with up to 2 substituents each selected independently from R¹²,
   phenyl optionally substituted with up to 2 substituents each selected independently from R¹², OH, COOR⁷, C(O)NHR⁷, S(O)₂(C₁-C₃)alkyl, NHS(O)₂(C₁-C₃)alkyl, N[(C₁-C₃)alkyl]₂, NH(C₁-C₃)alkyl,
      NHC(O)(C₁-C₃)alkyl, 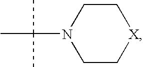
         and
      (C₁-C₃)alkoxy substituted with 1 substituent selected from N[(C₁-C₃)alkyl]₂, NH(C₁-C₃)alkyl, and 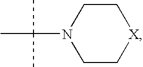
   indolyl optionally substituted with 1 or 2 substituents each selected independently from R¹², OH, C(O)O(C₁-C₄)alkyl,
      (C₁-C₃)alkyl substituted with 1 or 2 substituents each selected independently from OH, C(O)R⁸, (C₁-C₃)alkoxy, pyrrolidinyl, 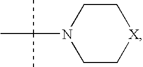
         imidazolyl, NH(C₁-C₃)alkyl, and N[(C₁-C₃)alkyl]₂, and
      (C₁-C₃)alkoxy substituted with 1 substituent selected from NH(C₁-C₃)alkyl, N[(C₁-C₃)alkyl]₂, pyrrolidinyl, imidazolyl, 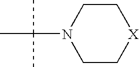
         and
      (C₁-C₃)alkoxy, and
      another heteroaryl optionally substituted with up to 3 substituents each independently selected from R¹²;
L is selected from CHR⁴, CHR⁵—CHR⁶, and CHR⁵—CH₂ —CHR⁶;
R¹ is selected from H, C(O)R¹⁰, C(O)OR⁷, tetrahydropyranyl, (C₃-C₆)cycloalkyl,
   phenyl optionally substituted with up to 2 substituents each independently selected from R¹²,
   pyridyl, optionally substituted with up to 2 substituents each independently selected from R¹²,
   S(O)₂-phenyl where said phenyl is optionally substituted with 1 or 2 substituents each independently selected from R¹², NH₂, NHC(O)(C₁-C₃)alkyl, NH(C₁-C₃)alkyl-N[(C₁-C₃)alkyl]₂, NH(C₁-C₃)alkyl-OH, COOH, OH, and (C₁-C₃)alkoxy substituted with 1 substituent selected from N[(C₁-C₃)alkyl]₂, OH, and 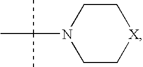
   S(O)₂(C₁-C₃)alkyl optionally substituted with one phenyl ring,
   (C₁-C₆)alkyl optionally substituted with 1 or 2 substituents each independently selected from OR¹¹, C(O)R¹⁰, C(O)OR⁷, N[(C₁-C₃)alkyl]₂,
      (C₃-C₆)cycloalkyl, dioxopyrrolidinyl, 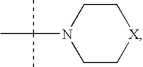
         glucopyranosyl, glucopyranosylamino,
      (C₁-C₃)alkoxy optionally substituted with 1 or 2 substituents each selected independently from OH, 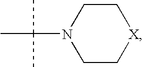
         and imidazolyl,
      O-phenyl optionally substituted with up to two substituents each independently selected from R¹²,
      NH₂ where one H is optionally replaced with one substituent selected from S(O)₂(C₁-C₃)alkyl, S(O)₂NH(C₁-C₃)alkyl, S(O)₂CF₃, C(O)R⁷, S(O)₂N[(C₁-C₃)alkyl]₂, C(O)O(C₁-C₄)alkyl, C(O)NH(C₁-C₄)alkyl, C(O)N[(C₁-C₃)alkyl]₂, 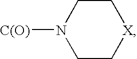
         and (C₁-C₄)alkyl optionally substituted with one OH group,
      phenyl optionally substituted with 1 or 2 substituents each independently selected from R¹², OH, S—(C₁-C₃)alkyl, C(O)NH₂, S(O)₂NH₂, C(O)N[(C₁-C₃)alkyl]₂, S(O(C₁-C₃)alkyl, S(O)₂NHC(O)(C₁-C₃)alkyl, C(O)(C₁-C₃)alkyl, C(O)NH(C₁-C₃)alkyl, NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHC(O)NH₂, S(O)₂N[(C₁-C₃)alkyl]₂, NHS(O)₂NH(C₁-C₃)alkyl, NHC(O)(C₁-C₃)alkyl, S(O)₂NH(C₁-C₃)alkyl optionally substituted with 1 substituent selected from (C₁-C₃)alkoxy, NH(C₁-C₃)alkyl, N[(C₁-C₃)alkyl]₂, and 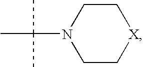
         (C₁-C₃)alkyl substituted with one substituent selected from NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHS(O)₂NH(C₁-C₃)alkyl, and NHC(O)(C₁-C₃)alkyl, and
         (C₁-C₃)alkoxy substituted with 1 substituent selected from OH, NH(C₁-C₃)alkyl, N[(C₁-C₃)alkyl]₂, (C₁-C₃)alkoxy, and 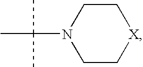
      pyrrolyl optionally substituted with one substituent selected from R¹², C(O)N[(C₁-C₃)alkyl]₂, C(O)NH(C₁-C₃)alkyl, C(O)(C₁-C₃)alkyl, 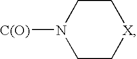
         and S(O)₂(C₁-C₃)alkyl,
      pyrazolyl optionally substituted with up to 3 substituents each selected independently from R¹², C(O)N[(C₁-C₃)alkyl]₂, C(O)NH(C₁-C₃)alkyl, and 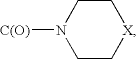
         and
      another heteroaryl optionally substituted with up to two substituents each independently selected from R¹²;
R² is in each instance selected independently from (C₁-C₃)alkyl, halo, (C₁-C₃)alkoxy, CF₃, NO₂, NH₂, CN, and COOH;
R³ is selected from H, (C₁-C₃)alkyl, and halo;
R⁴ is selected from H and (C₁-C₃)alkyl-OH;
R⁵ is selected from H, OH and (C₁-C₃)alkyl;
R⁶ is selected from H, C(O)OR⁷, C(O)R⁹, and (C₁-C₆)alkyl optionally substituted with one substituent selected from OH, NHS(O)₂(C₁-C₃)alkyl, and NHC(O)(C₁-C₃)alkyl;
R⁷ is selected from H and (C₁-C₄)alkyl;
R⁸ is selected from OH, NH₂, N[(C₁-C₃)alkyl]₂, morpholinyl, and pyrrolidinyl;
R⁹ is selected from NH₂, morpholinyl, N[(C₁-C₃)alkyl]₂, and NH(C₁-C₃)alkyl optionally substituted with one substituent selected from OH, COOH, and N[(C₁-C₃)alkyl]₂;
R¹⁰ is selected from (C₃-C₆)cycloalkyl, morpholinyl, N[(C₁-C₄)alkyl]₂, (C₁-C₃)alkoxy,
   heteroaryl optionally substituted with 1 or 2 substituents each independently selected from (C₁-C₃)alkyl, (C₁-C₃)alkoxy, OH, halo and CF₃,
   phenyl optionally substituted with 1 or 2 substituents each independently selected from (C₁-C₃)alkyl, (C₁-C₃)alkoxy, OH, halo and CF₃,
   (C₁-C₃)alkyl optionally substituted with one substituent selected from phenyl, imidazolyl, and 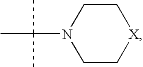
   NH(C₁-C₄)alkyl optionally substituted with 1 phenyl ring optionally substituted with 1 or 2 substituents each independently selected from (C₁-C₃)alkyl, (C₁-C₃)alkoxy, halo and CF₃, and
   NH-phenyl where said phenyl is optionally substituted with 1 or 2 substituents each independently selected from (C₁-C₃)alkyl, (C₁-C₃)alkoxy, halo and CF₃;
R¹¹ is selected from H, C(O)N[(C₁-C₃)alkyl]₂, C(O)-pyrrolidinyl, C(O)NH-phenyl, and C(O)NH(C₁-C₃)alkyl optionally substituted with 1 phenyl ring;
R¹² is selected from (C₁-C₆)alkyl, (C₁-C₃)alkoxy, halo, NO₂, CN, CF₃, O—CF₃, and phenyl optionally substituted with up to 2 substituents each selected independently from halo, (C₁-C₃)alkyl, and (C₁-C₃)alkoxy;
X is selected from O, S, CH₂, and NH, and when X is NH, the H on NH is optionally replaced with C(O)(C₁-C₃)alkyl, S(O)₂(C₁-C₃)alkyl, or (C₁-C₆)alkyl
   and when X is O, S, or CH₂, the 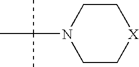
      moiety is optionally substituted by replacing any H atom in the 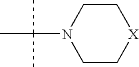
      moiety with (C₁-C₄)alkyl;
m is selected from 0, 1 and 2;
n is selected from 1 and 2; [symbol] is is selected from a double bond and a single bond; or a pharmaceutically acceptable salt, ester or carbonate thereof.

The terms identified above have the following meaning throughout:

The term “optionally substituted” means that the moiety so modified may have from none to up to at least the highest number of substituents indicated. The substituent may replace any H atom on the moiety so modified as long as the replacement is chemically possible and chemically stable. When there are two or more substituents on any moiety, each substituent is chosen independently of any other substituent and can, accordingly, be the same or different.

The terms “(C₁-C₃)alkyl”, “(C₁-C₄)alkyl” and “(C₁-C₆)alkyl”, mean linear or branched saturated carbon groups having from about 1 to about 3, about 4, or about 6 C atoms, respectively. Such groups include but are not limited to methyl, ethyl, n-propyl, isopropyl, n-butyl, isobutyl, sec-butyl, tert-butyl, and the like.

The term “(C₁-C₃)alkoxy” means a linear or branched saturated carbon group having from about 1 to about 3 C atoms, said carbon group being attached to an O atom. The O atom is the point of attachment of the alkoxy substituent to the rest of the molecule. Such groups include but are not limited to methoxy, ethoxy, n-propoxy, isopropoxy, and the like.

When an alkyl or an alkoxy group is “optionally substituted”, that means that any H atom on any C atom in the group is replaced with a recited substituent as long as the substitution is chemically appropriate for the C atom's location in the molecule, and as long as only about the maximum number of substituents recited replace H atoms in any specific alkoxy group.

The term “(C₃-C₆)cycloalkyl” means the monocyclic analogs of an alkyl group having from about 3 to about 6 C atoms, as defined above. Examples of (C₃-C₆)cycloalkyl groups include but are not limited to cyclopropyl, cyclobutyl, cyclopentyl, cyclohexyl, and the like.

The term “halo” means an atom selected from Cl, Br, F and I, where Cl, Br and F are preferred.

When “(O)” is used in a chemical formula, it means =O. For example, “C(O)” means a carbonyl group and “S(O)₂” means a sulfonyl group.

The formula “N[C₁-C₃)alkyl]₂” means that each of the 2 possible alkyl groups attached to the N atom are selected independently from the other so that they may be the same or they may be different.

In the case of (R²)ₘ, when m is 1 or 2, R² is in each instance attached to the core molecule at any available C atom on the phenyl ring. That is, when m is 1, R² is attached at any one of the three available C atoms of the phenyl ring. When m is 2, each R² group is attached to a separate available C atom selected form the three available C atoms of the phenyl ring, and each R² group is selected independently from the other.

The terms “heteroaryl” and “another heteroaryl” (hereafter, severally and collectively “another/heteroaryl”) each means an aromatic mono or fused bicyclic ring containing about 5 to about 10 atoms, 1, 2, 3, or 4 of which are each independently selected from N, O and S, the remaining atoms being C, as described further below.

When another/heteroaryl is an aromatic monocyclic ring containing 5 atoms, 1, 2, 3, or 4 of the atoms are each independently selected from N, O and S, and the remaining atoms are C, with the proviso that there is no more than one O atom or one S atom in any ring. The 5 membered heteroaryl is attached to the core molecule at any available C or N atom, and any substituent may be attached to the heteroaryl at any available C or N atom with the proviso that halo, NO₂, CN, O—CF₃, or alkoxy substituents are attached to the ring at any of the ring's available C atoms only. Such 5-membered heteroaryl groups include pyrrolyl, furanyl, thienyl, imidazolyl, pyrazolyl, thiazolyl, isothiazolyl, oxazolyl, isoxazolyl, isothiazolyl, triazolyl, oxadiazolyl, tetrazolyl, and thiadiazolyl, and the like, in all their possible isomeric forms.

When another/heteroaryl is an aromatic monocyclic ring containing 6 atoms, 1 or 2 of the atoms in the ring are N, and the remaining atoms are C. The moiety is attached to the core molecule at any available C atom, and any substituent may be attached to the 6 membered heteroaryl at any available C atom. Such groups include pyridinyl, pyrimidinyl, pyridazinyl, pyrazinyl, and the like, in any possible isomeric form.

When another/heteroaryl is a fused bicyclic ring, it has from 9 to 10 atoms divided into 2 rings that are fused together and 1, 2, 3, or 4 of which are each independently selected from N, O and S with the proviso that there can be no more than one O atom or one S atom in any fused bicyclic ring. The complete fused bicyclic ring system is aromatic. The heteroatoms may be located at any available position on the fused bicyclic moiety. A fused bicyclic heteroaryl is attached to the core molecule through any available C or N atom, and is optionally substituted at any available C or N atom(s) with the recited substituents with the exception that halo, NO₂, CN, O—CF₃, or alkoxy substituents are attached to the ring at any of the ring's available C atoms only. Bicyclic heteroaryl groups include −5-6, and —6-6 fused bicycles. The fused bicycles include, but are not limited to benzofuranyl, quinolinyl, isoquinolinyl, naphthyridinyl, indolyl, indazolyl, isoindolyl, benzoxazolyl, benzothiazolyl, benzimidazolyl, benzothienyl, benzotriazolyl and the like, in any possible isomeric form.

When W is another heteroaryl, indolyl is not included in this group. When W is optionally substituted indolyl, the indolyl moiety may be attached to the rest of the molecule at any available C or N atom, and it may be optionally substituted at any available C or N atom in the indolyl moiety.

When R¹ is (C₁-C₆)alkyl substituted with another heteroaryl, pyrrolyl and pyrazolyl are not included in the another heteroaryl group. When R¹ is (C₁-C₆)alkyl substituted with optionally substituted pyrrolyl or optionally substituted pyrazolyl, the said pyrrolyl or pyrazolyl may be attached to the rest of the molecule at any available C or N atom, and it may be optionally substituted at any available C or N atom on the ring with the exception that halo, NO₂, CN, O—CF₃, or alkoxy substituents are attached to the ring at any of the ring's available C atoms only.

When a glucopyranosyl group is attached to the rest of the molecule, it is attached through any O atom bonded to the groups pyranyl ring, and when a glucopyranosylamino group is attached to the rest of the molecule, it is attached through its N atom.

When a phenyl ring is substituted with one or more substituent, the substituent(s) may be attached to the phenyl ring at any available C atom. When there is more than 1 substituent on a phenyl ring, each is selected independently from the other so that they may be the same or different. 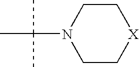
means optionally substituted morpholinyl, thiomorpholinyl, piperidinyl or piperazinyl. A 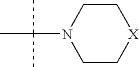
ring may be attached to the rest of the molecule through any available N atom in the 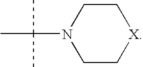
When a 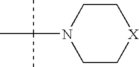
ring is substituted, the substituent(s) is/are attached to the ring at any of the ring's available C or N atom(s). When there is more than 1 substituent on a ring, each is selected independently from the other so that they may be the same or different.

When n is 1, the W-L-N(R¹)— side chain may be attached to the rest of the molecule at the C1, C2, or C3 atom, preferably at the C1 or C2 atom where the carbon atoms are numbered as follows:

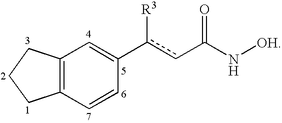

When n is 2, the W-L-N(R¹)— side chain may be attached to the rest of the molecule at C5, C6, C7, or C8 atom, preferably at C5, or C6 atom where the carbon atoms are numbered as follows:

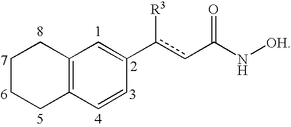

When L is CHR⁵—CHR⁶ or CHR⁵—CH₂—CHR⁶, W is linked to these groups at the CHR⁵ carbon atom and N(R¹) is linked to these groups at the CHR⁶ carbon atom.

Representative compounds of Formula I are shown in Table I. Those compound examples that have characterization data such as HPLC retention time and/or M+H mass spectroscopy data listed were actually synthesized. Those that do not have characterization data were not synthesized; however, they can be synthesized by following procedures that are well known to those skilled in the art and/or procedures that are disclosed in this application.

TABLE I

| | | | HPLC RT | |
|---|---|---|---|---|
| Compound | | Synthetic | (min) | |
| Example | Structure | sequence | (method) | M + H |
| 1 | 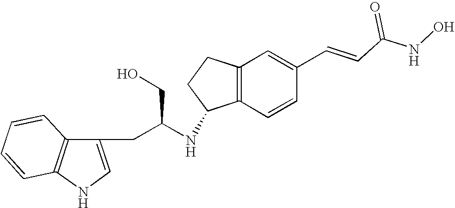 | D, E1, 1 | 1.65 (A) | 391.9 |
| 2 | 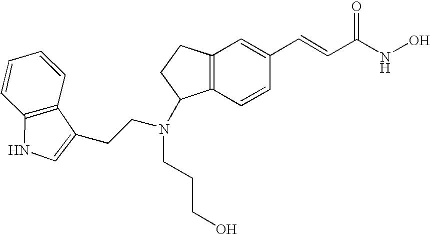 | D, E, G2, J3, 2 | 1.53 (A) | 420.0 |
| 3 | 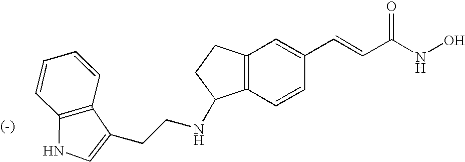 | D, E, I1, 3 | 1.70 (A) | 362.0 |
| 4 | 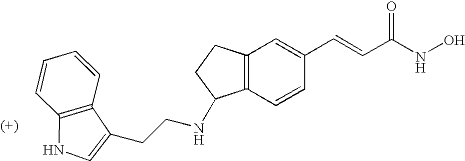 | D, E, I2, 4 | 1.68 (A) | 362.0 |
| 5 | 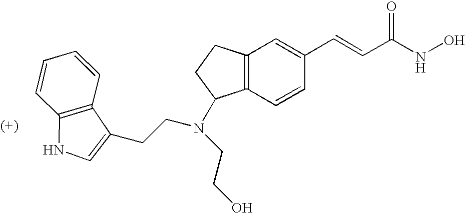 | D, E, G, J, H2, 5 | 1.52 (A) | 406.0 |
| 6 | 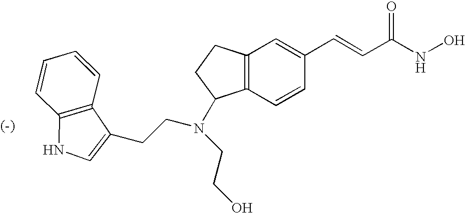 | D, E, G, J, H1, 6 | 1.51 (A) | 406.0 |
| 7 | 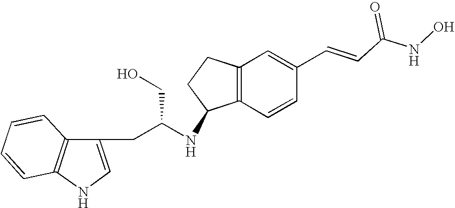 | D, E3, 7 | 1.63 (A) | 391.9 |
| 8 | 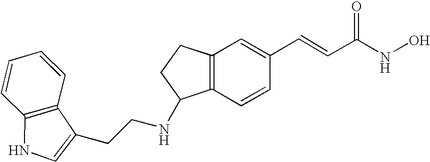 | D, E, 8 | 1.73 (A) | 362.0 |
| 9 | 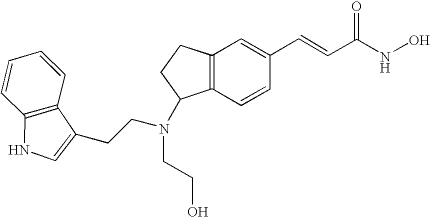 | D, E, G, J, 9 | 1.54 (A) | 406.0 |
| 10 | 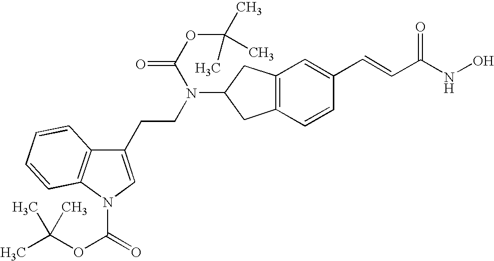 | C, G1 (via A), A2, D1, 10 | 3.72 (A) | 561.9 |
| 11 | 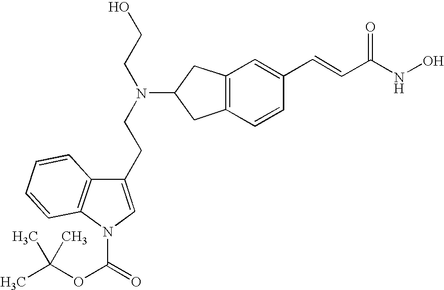 | C, F, F1 (via B), J2, D2, 11 | 2.17 (B) | 406.5 (—BOC) |
| 12 | 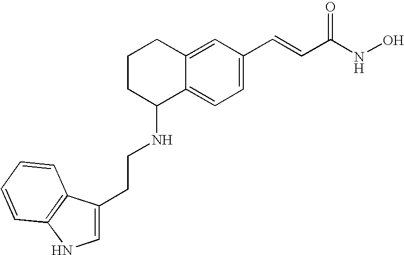 | K, L, E4, 12 | 1.98 (A) | 376.0 |
| 13 | 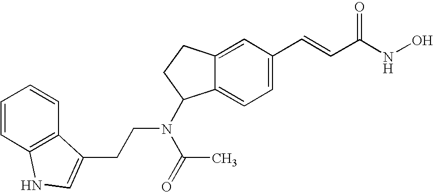 | D, E, M, 13 | 2.30 (A) | 403.9 |
| 14 | 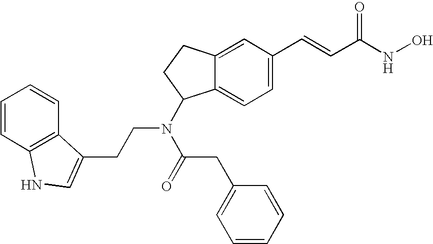 | D, E, M1, 14 | 2.76 (A) | 480.0 |
| 15 | 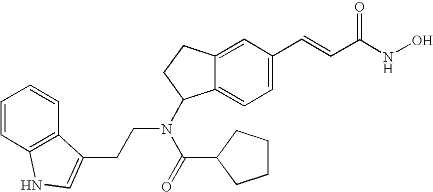 | D, E, M2, 15 | 2.82 (A) | 458.0 |
| 16 | 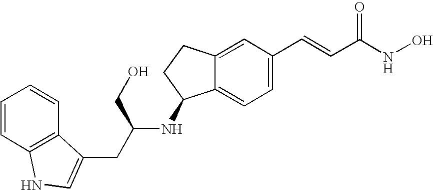 | D, E2, 16 | 1.72 (A) | 392.0 |
| 17 | 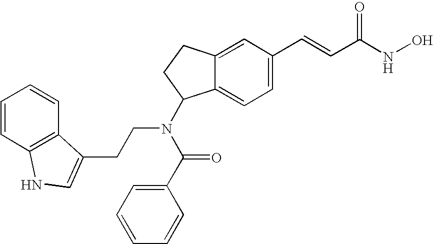 | D, E, M3, 17 | 2.68 (A) | 465.9 |
| 18 | 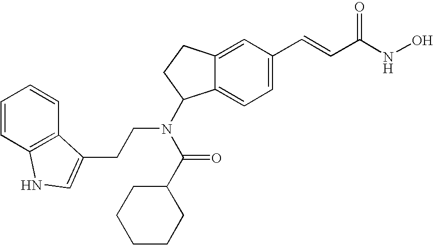 | D, E, M4, 18 | 2.91 (A) | 472.1 |
| 19 | 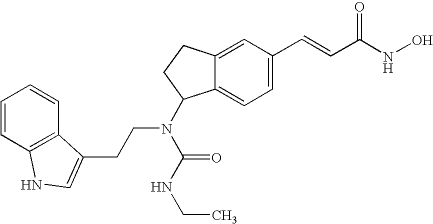 | D, E, N, 19 | 2.43 (A) | 432.9 |
| 20 | 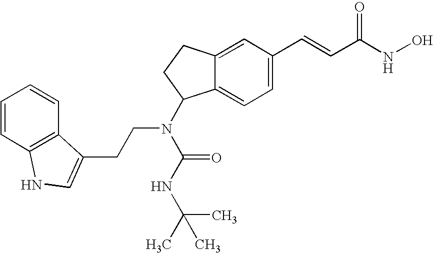 | D, E, N1, 20 | 2.80 (A) | 461.0 |
| 21 | 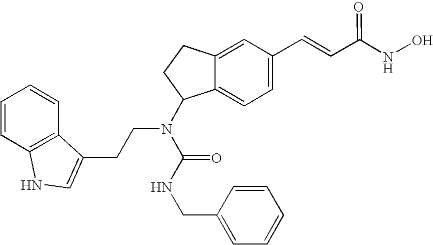 | D, E, N2, 21 | 2.79 (A) | 494.9 |
| 22 | 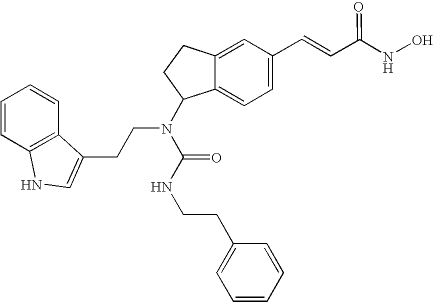 | D, E, N3, 22 | 2.79 (A) | 508.9 |
| 23 | 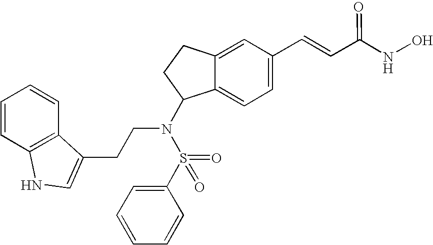 | D, E, O, 23 | 3.11 (A) | 501.9 |
| 24 | 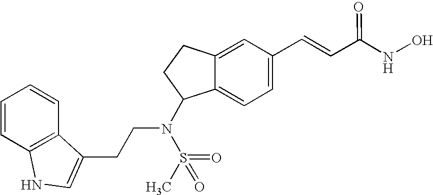 | D, E, O1, 24 | 2.49 (A) | 439.9 |
| 25 | 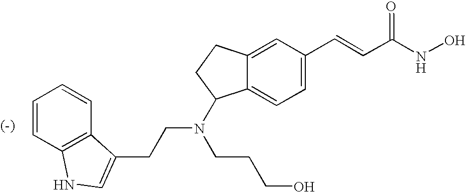 | D, E, G2, J3, P1, 25 | 1.68 (A) | 420.0 |
| 26 | 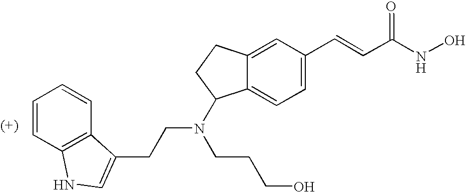 | D, E, G2, J3, P2, 26 | 1.68 (A) | 420.0 |
| 27 | 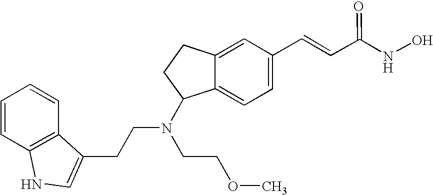 | D, E, G3, 27 | 1.83 (A) | 420.0 |
| 28 | 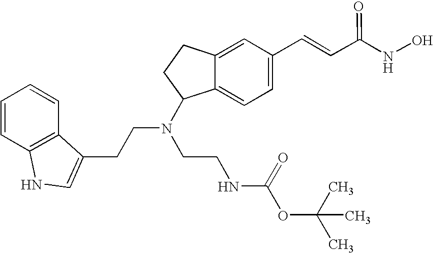 | D, E, F2, 28 | 2.15 (A) | 504.9 |
| 29 | 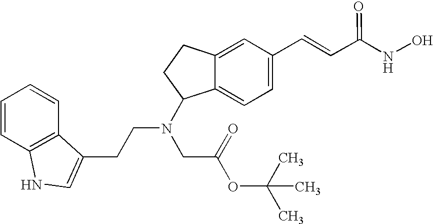 | D, E, G4, 29 | 2.27 (A) | 476.1 |
| 30 | 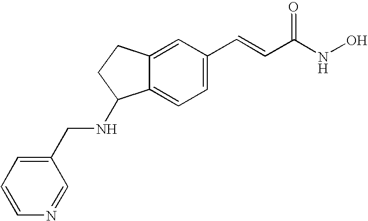 | D, Q, 30 | 1.03 (A) | 310.0 |
| 31 | 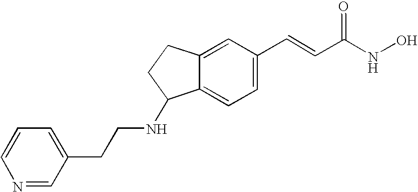 | D, Q1, 31 | 0.76 (A) | 324.1 |
| 32 | 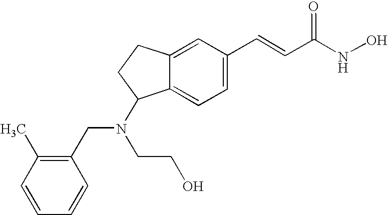 | D, Q2, F3, J4, 32 | 1.51 (A) | 367.0 |
| 33 | 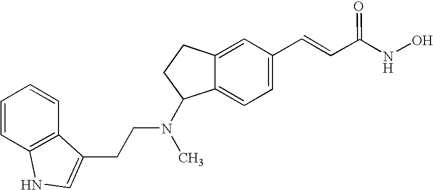 | D, Q3, 33 | 1.73 (A) | 376.0 |
| 34 | 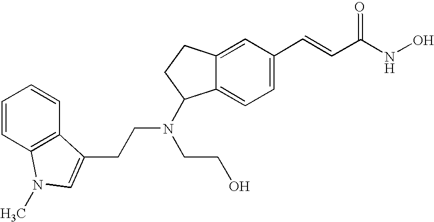 | D, E, G, G5, J5, 34 | 1.82 (A) | 420.0 |
| 35 | 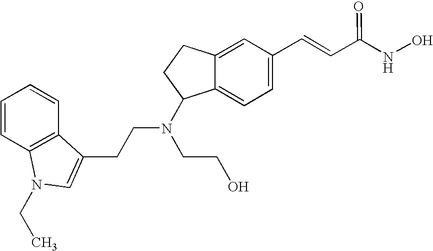 | D, E, G, G6, J6, 35 | 1.93 (A) | 434.0 |
| 36 | 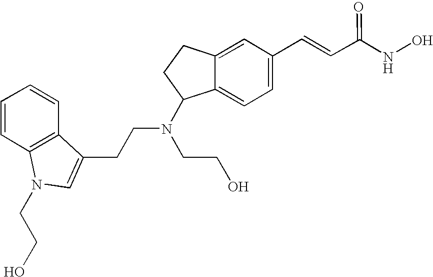 | D, E, G, G7, J7, 36 | 1.62 (A) | 450.0 |
| 37 | 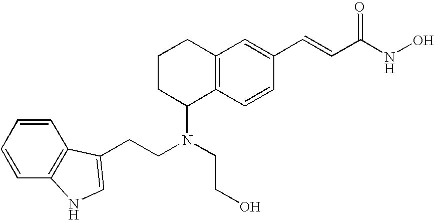 | K, L, E4, F4, J8, 37 | 1.95 (A) | 420.0 |
| 38 | 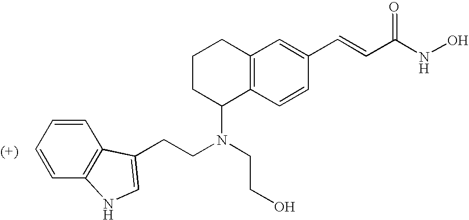 | K, L, E4, F4, J8, R1, 38 | 1.64 (A) | 420.0 |
| 39 | 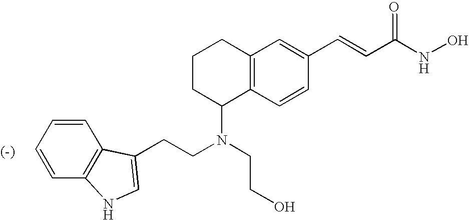 | K, L, E4, F4, J8, R2, 39 | 1.65 (A) | 419.9 |
| 40 | 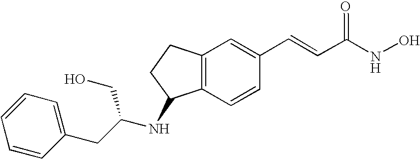 | D, E5, 40 | 1.44 (A) | 353.0 |
| 41 | 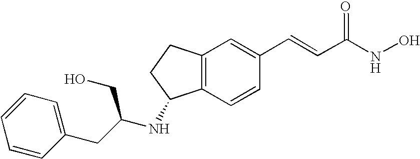 | D, E6, 41 | 1.44 (A) | 352.9 |
| 42 | 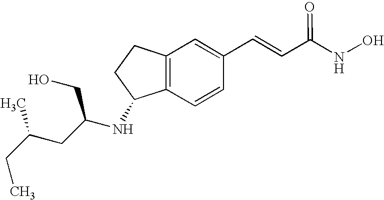 | D, E7, 42 | 1.23 (A) | 319.0 |
| 43 | 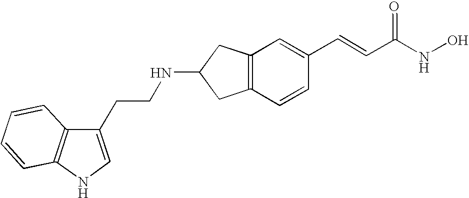 | C, G1 (via A), A2, D1, 10, 43 | 1.70 (A) | 362.1 |
| 44 | 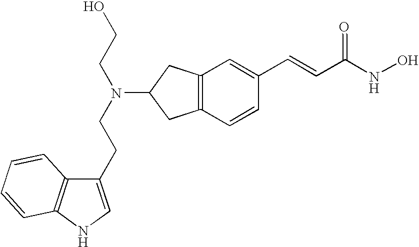 | C, F, F1, (via B), Jd, D2, 11, 44 | 1.65 (A) | 406.1 |
| 45 | 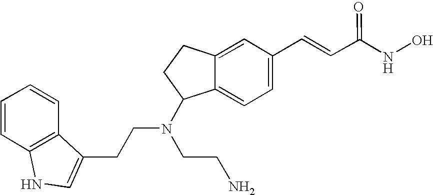 | D, E, F2, 28, 45 | 1.39 (A) | 404.7 |
| 46 | 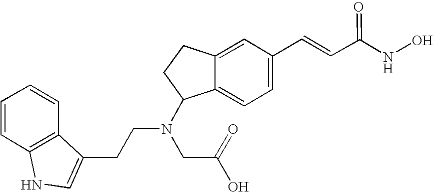 | D, E, G4, 29, 46 | 1.90 (A) | 420.0 |
| 47 | 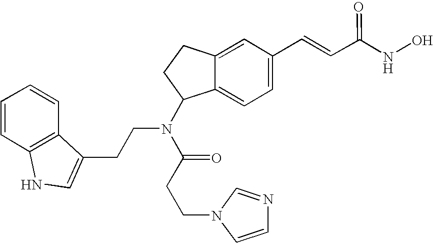 | | | |
| 48 | 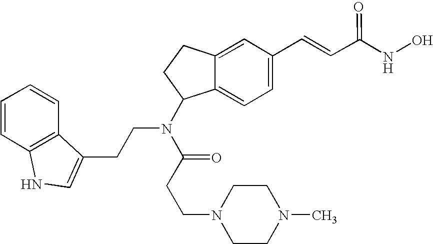 | | | |
| 49 | 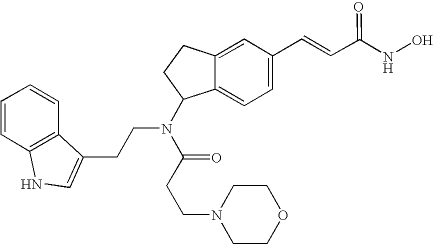 | | | |
| 50 | 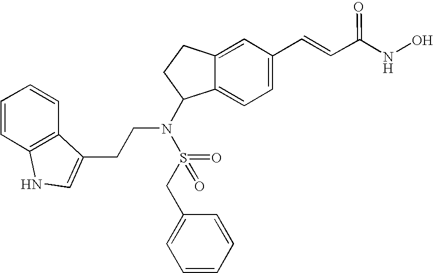 | | | |
| 51 | 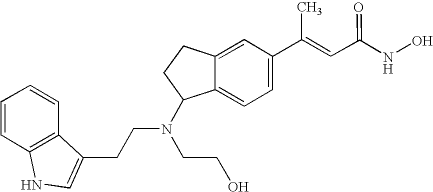 | D3, Q7, G8, J9, 51 | 1.67 (A) | 420.2 |
| 52 | 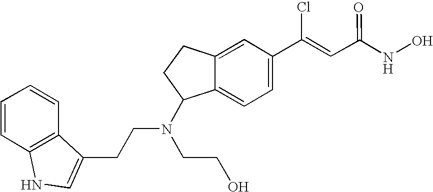 | | | |
| 53 | 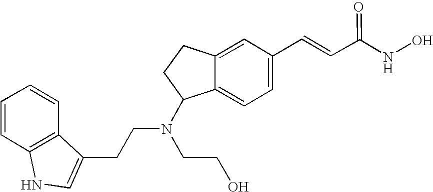 | D, E, G, J, S, 53 | 1.69 (A) | 408.0 |
| 54 | 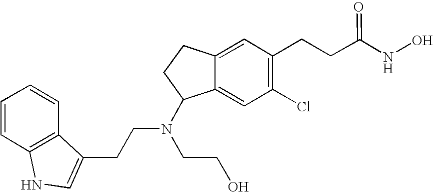 | | | |
| 55 | 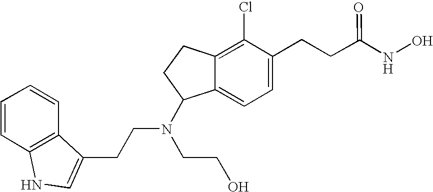 | | | |
| 56 | 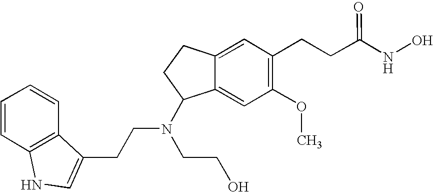 | | | |
| 57 | 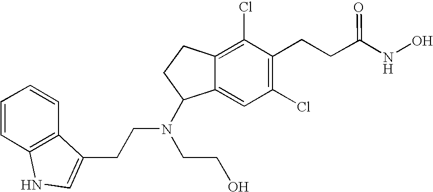 | | | |
| 58 | 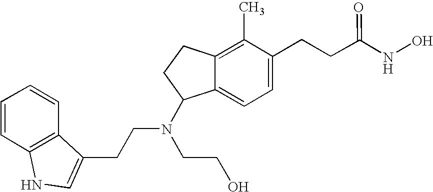 | | | |
| 59 | 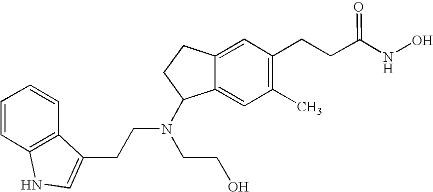 | | | |
| 60 | 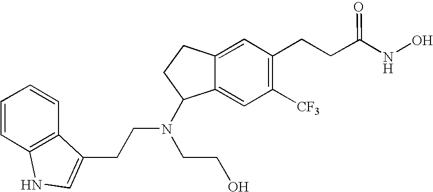 | | | |
| 61 | 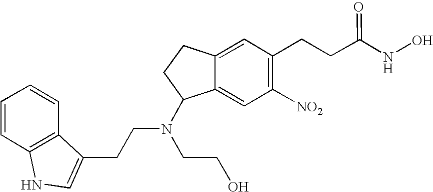 | | | |
| 62 | 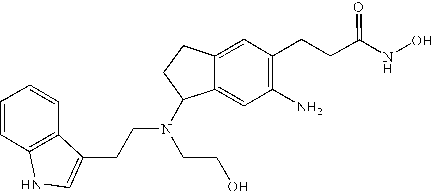 | | | |
| 63 | 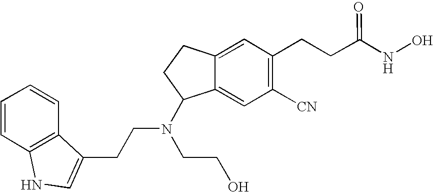 | | | |
| 64 | 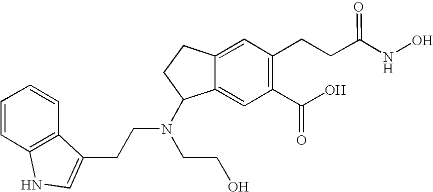 | | | |
| 65 | 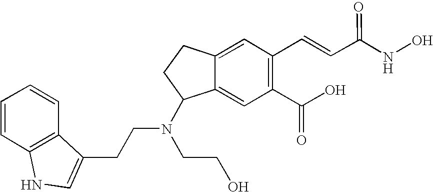 | | | |
| 66 | 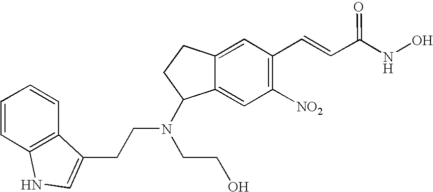 | | | |
| 67 | 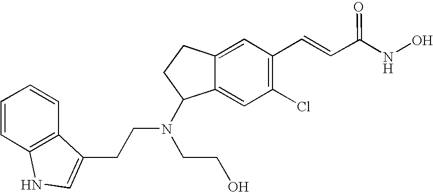 | | | |
| 68 | 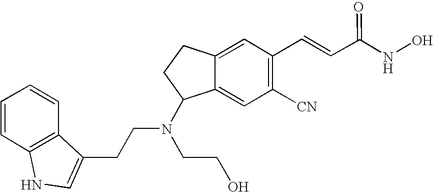 | | | |
| 69 | 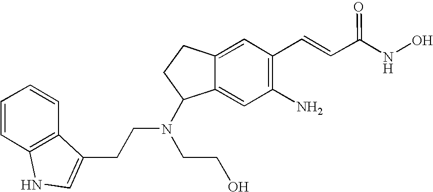 | | | |
| 70 | 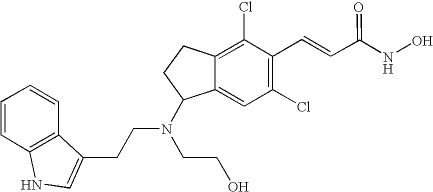 | | | |
| 71 | 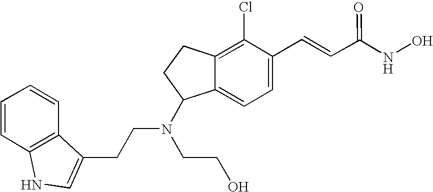 | | | |
| 72 | 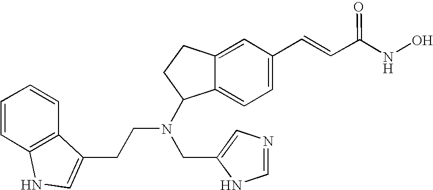 | D, E, F5, 72 | 1.66 (A) | 442.0 |
| 73 | 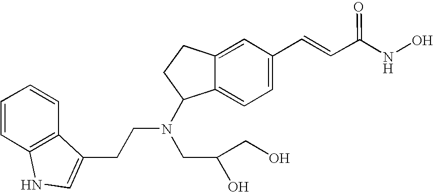 | | | |
| 74 | 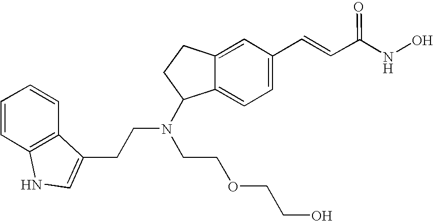 | D, E, G12, 74 | 1.13 (A) | 450.1 |
| 75 | 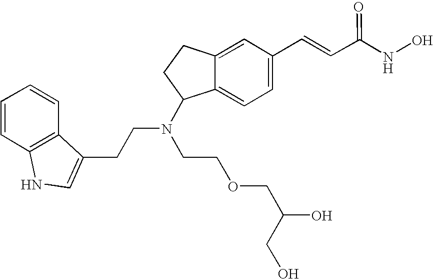 | | | |
| 76 | 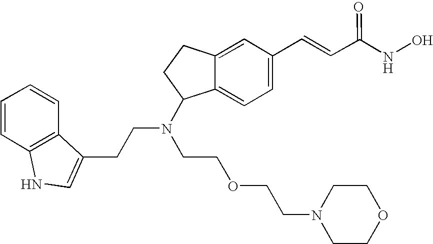 | | | |
| 77 | 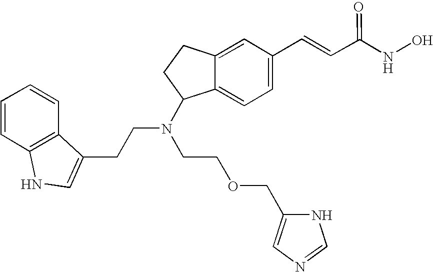 | | | |
| 78 | 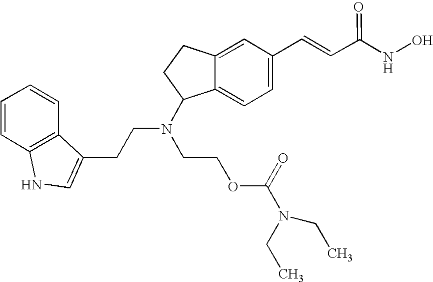 | | | |
| 79 | 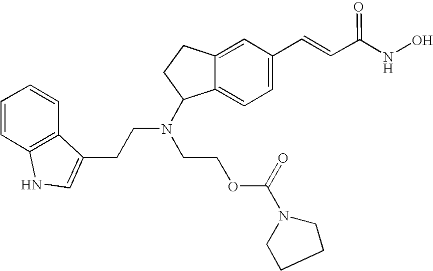 | | | |
| 80 | 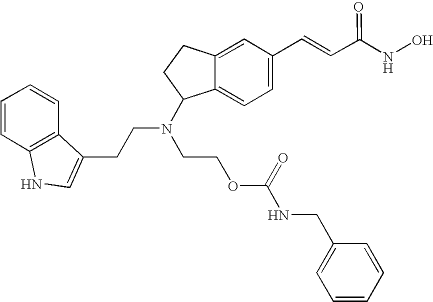 | | | |
| 81 | 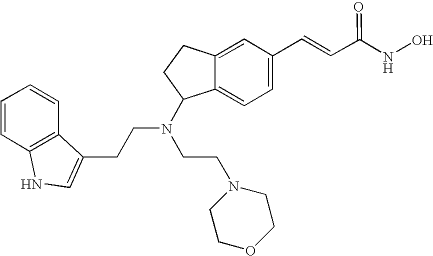 | D, E, G9, 81 | 1.82 (A) | 475.2 |
| 82 | 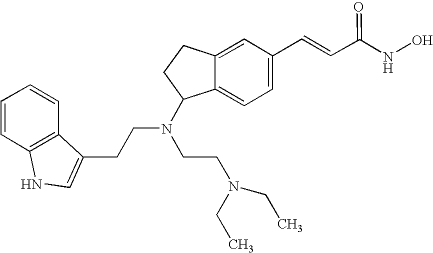 | | | |
| 83 | 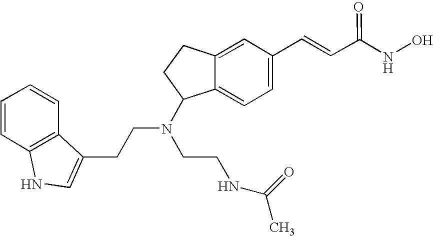 | D, E, F2, T, M5, 83 | 1.33 (A) | 447.1 |
| 84 | 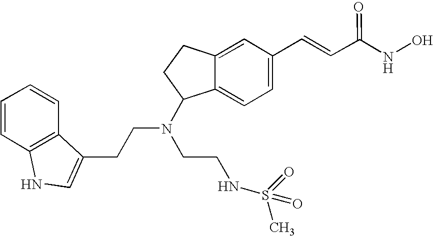 | D, E, F2, T, O2, 84 | 0.96 (A) | 486.0 |
| 85 | 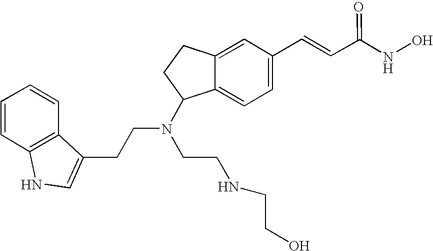 | D, E, F2, T, F99, J52, 85 | 1.38 (A) | 449.2 |
| 86 | 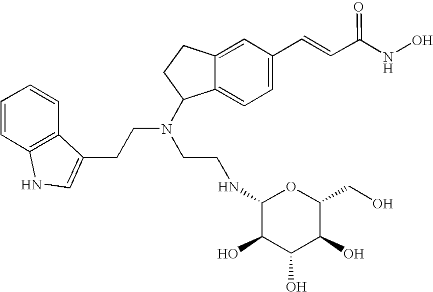 | | | |
| 87 | 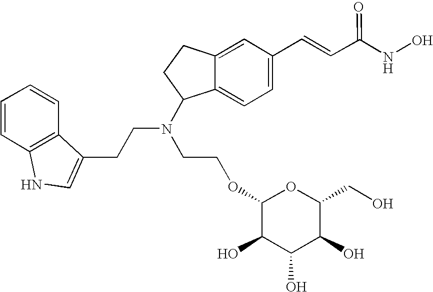 | | | |
| 88 | 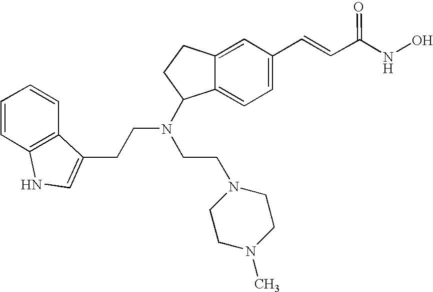 | | | |
| 89 | 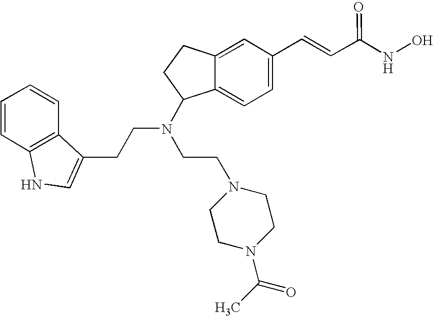 | | | |
| 90 | 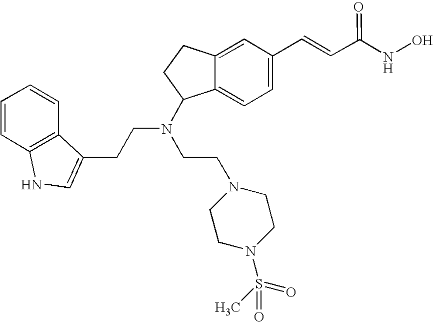 | | | |
| 91 | 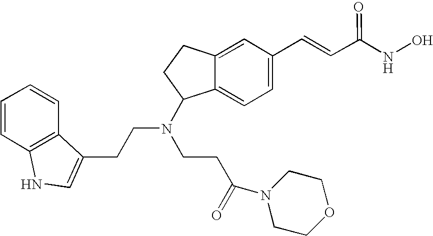 | | | |
| 92 | 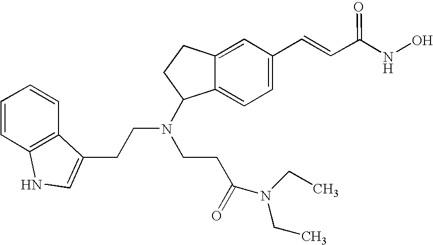 | | | |
| 93 | 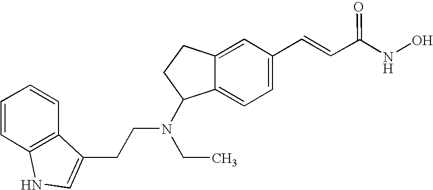 | D, E, F6, 93 | 1.77 (A) | 390.0 |
| 94 | 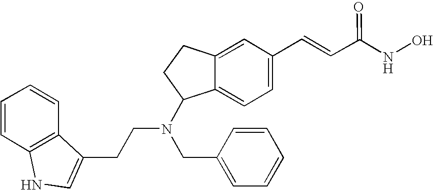 | D, E, F7, 94 | 2.29 (A) | 452.0 |
| 95 | 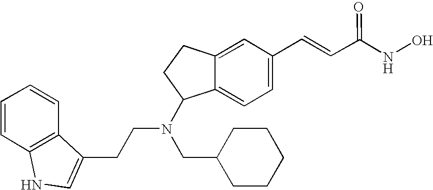 | D, E, F8, 95 | 2.40 (A) | 458.1 |
| 96 | 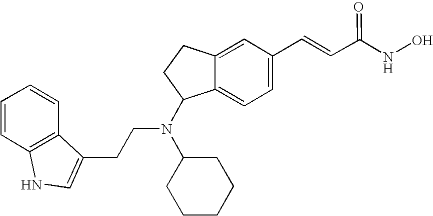 | | | |
| 97 | 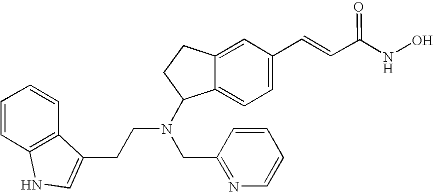 | | | |
| 98 | 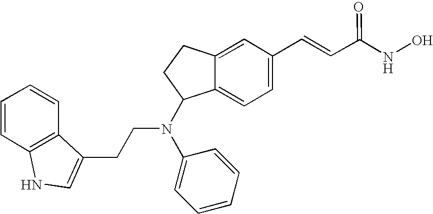 | | | |
| 99 | 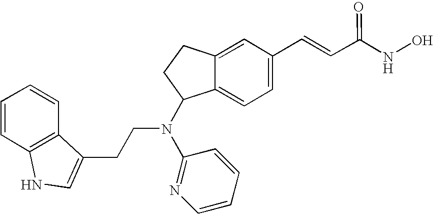 | | | |
| 100 | 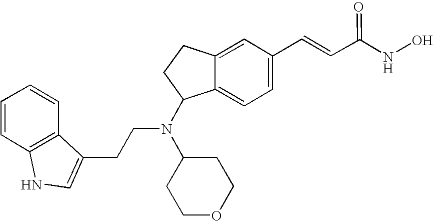 | | | |
| 101 | 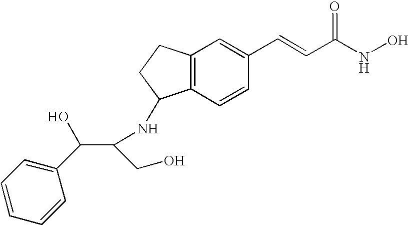 | | | |
| 102 | 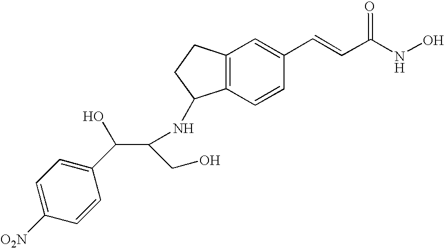 | | | |
| 103 | 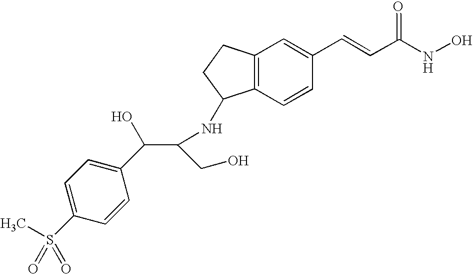 | | | |
| 104 | 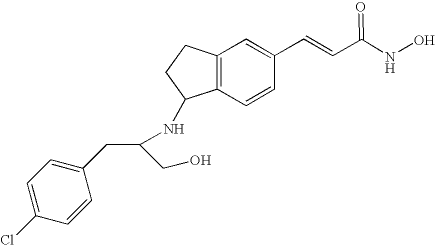 | | | |
| 105 | 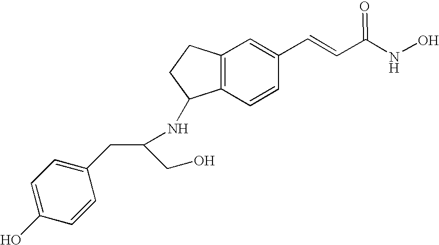 | | | |
| 106 | 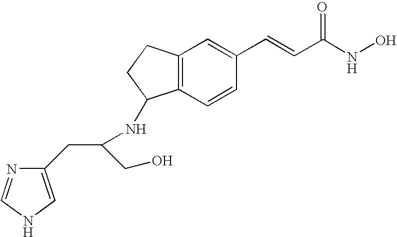 | | | |
| 107 | 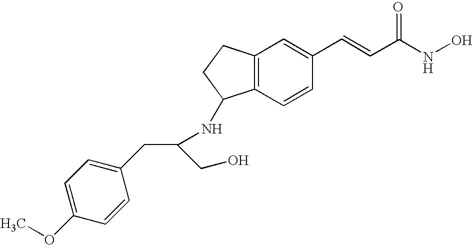 | | | |
| 108 | 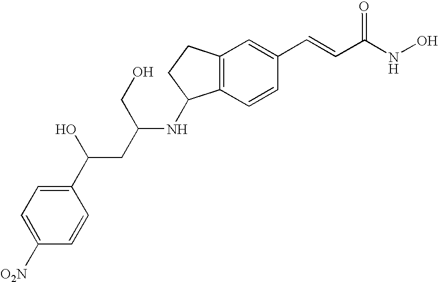 | | | |
| 109 | 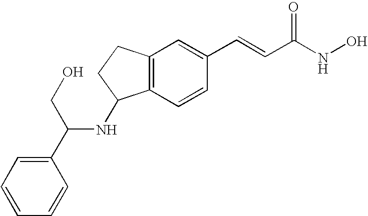 | | | |
| 110 | 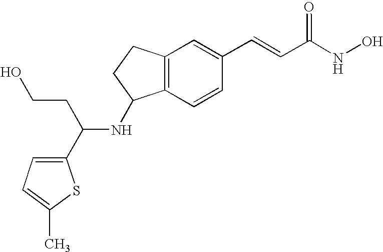 | | | |
| 111 | 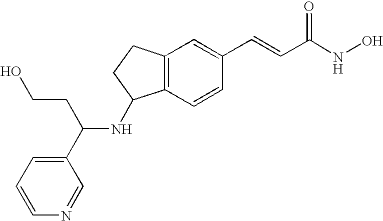 | | | |
| 112 | 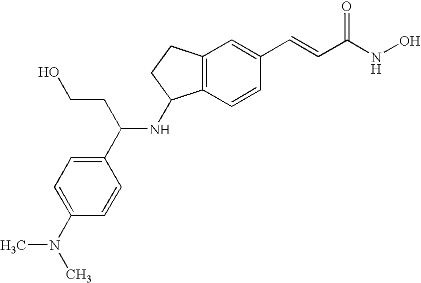 | | | |
| 113 | 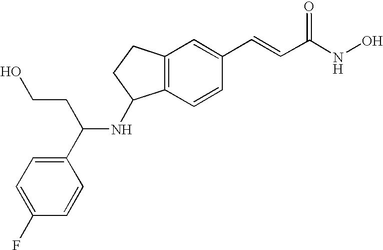 | | | |
| 114 | 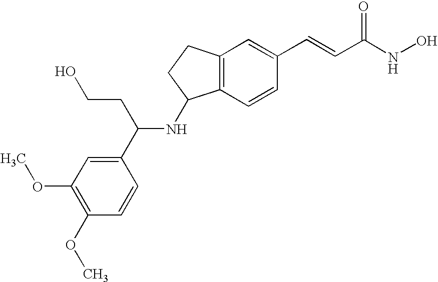 | | | |
| 115 | 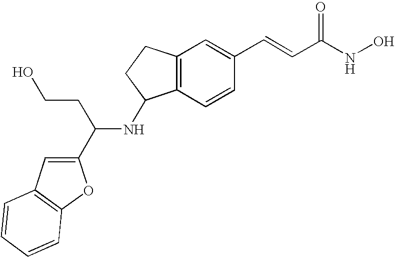 | | | |
| 116 | 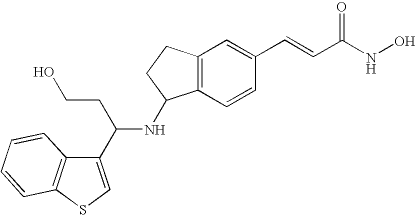 | | | |
| 117 | 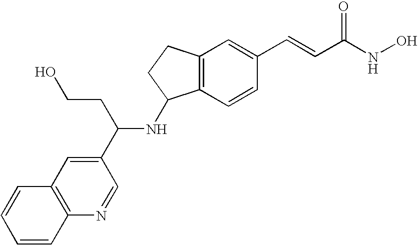 | | | |
| 118 | 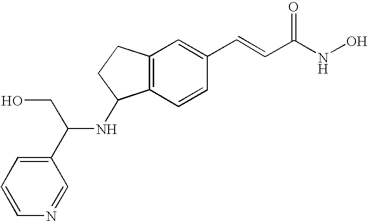 | | | |
| 119 | 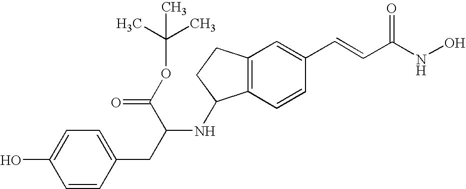 | | | |
| 120 | 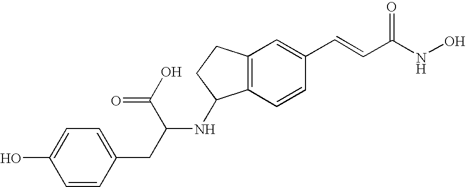 | | | |
| 121 | 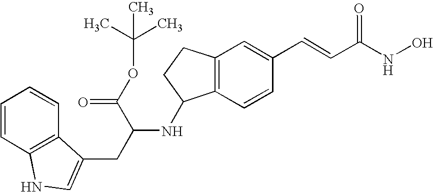 | | | |
| 122 | 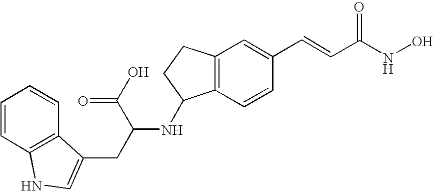 | | | |
| 123 | 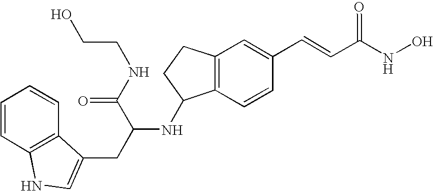 | | | |
| 124 | 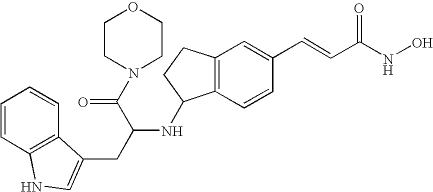 | | | |
| 125 | 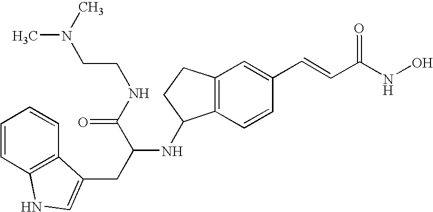 | | | |
| 126 | 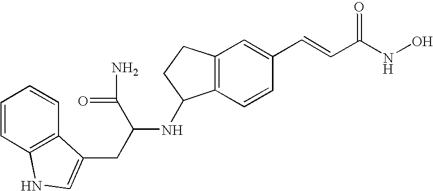 | | | |
| 127 | 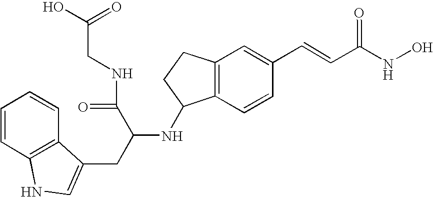 | | | |
| 128 | 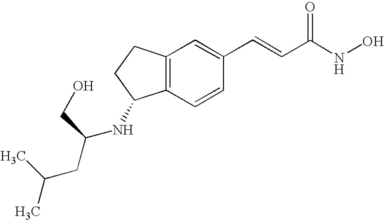 | D, E8, 128 | 1.30 (A) | 319.0 |
| 129 | 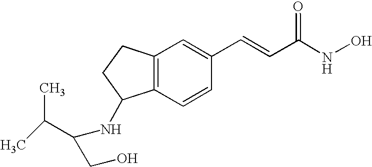 | | | |
| 130 | 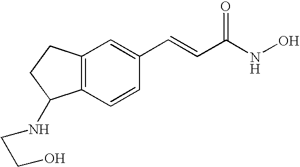 | | | |
| 131 | 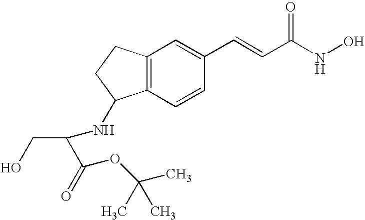 | | | |
| 132 | 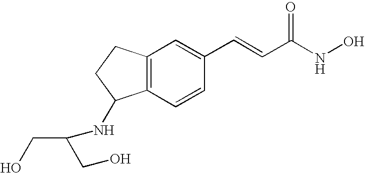 | | | |
| 133 | 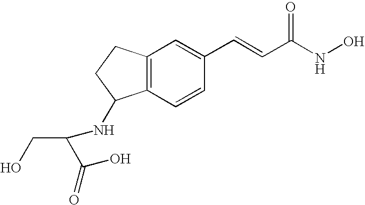 | | | |
| 134 | 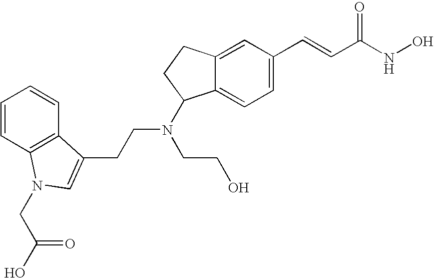 | | | |
| 135 | 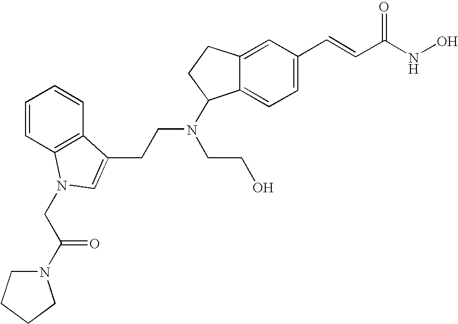 | | | |
| 136 | 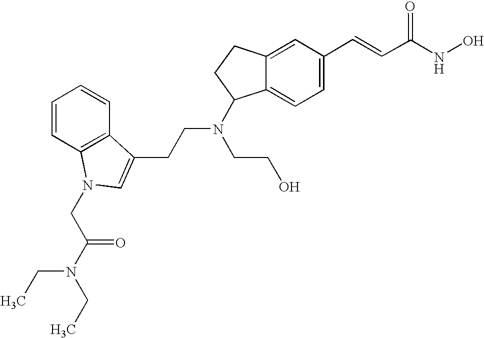 | | | |
| 137 | 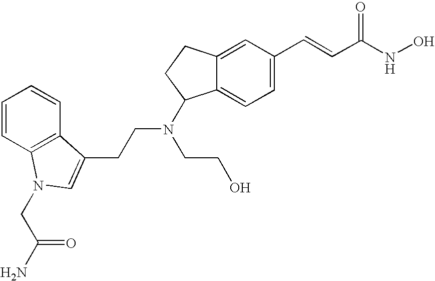 | | | |
| 138 | 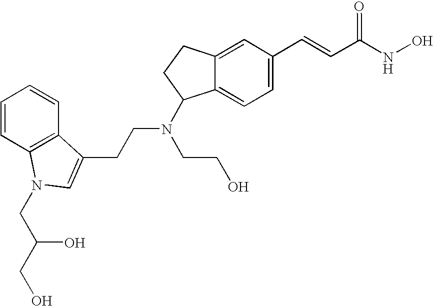 | | | |
| 139 | 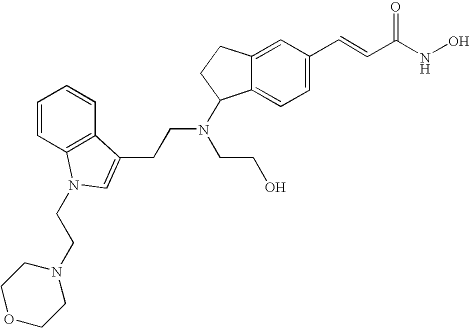 | | | |
| 140 | 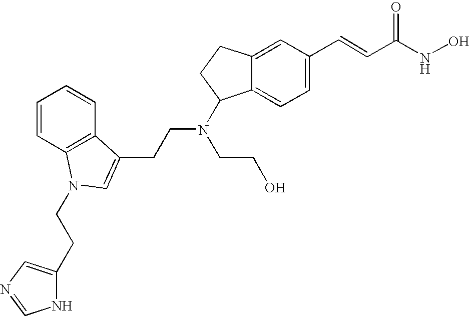 | | | |
| 141 | 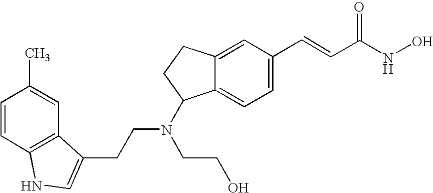 | | | |
| 142 | 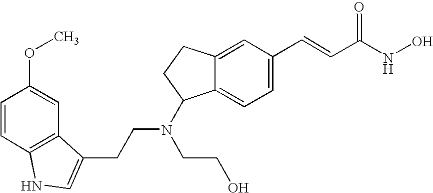 | D, Q4, F9, J10, 142 | 1.21 (A) | 436.0 |
| 143 | 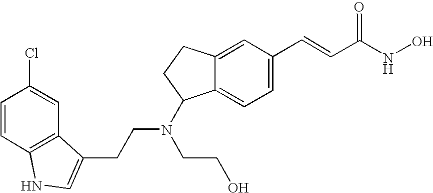 | D, Q5, F10, J11, 143 | 1.82 (A) | 439.9 |
| 144 | 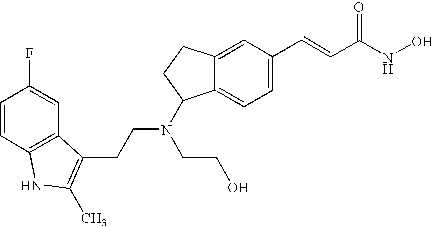 | D, Q6, F11, J12, 144 | 1.82 (A) | 437.9 |
| 145 | 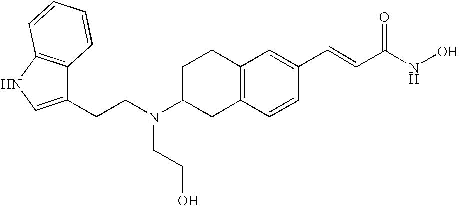 | | | |
| 146 | 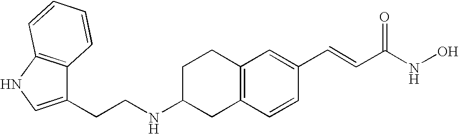 | | | |
| 147 | 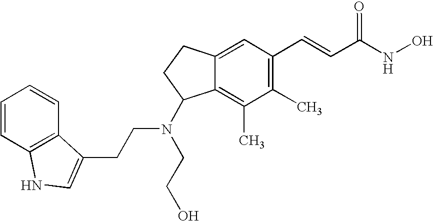 | | | |
| 148 | 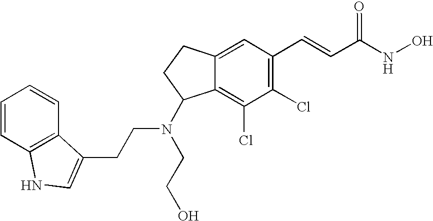 | | | |
| 149 | 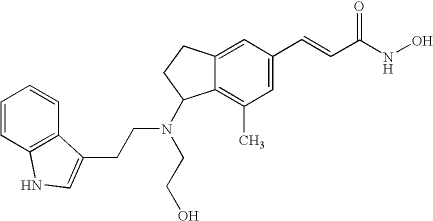 | | | |
| 150 | 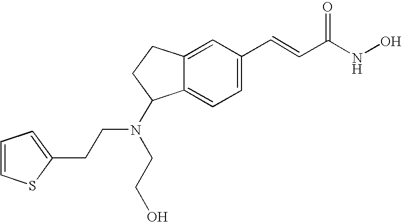 | | | |
| 151 | 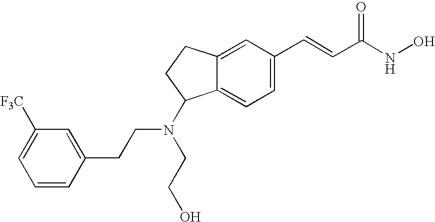 | | | |
| 152 | 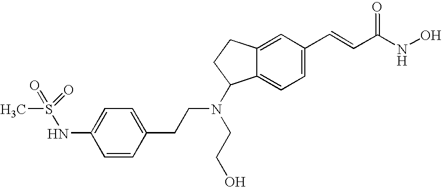 | | | |
| 153 | 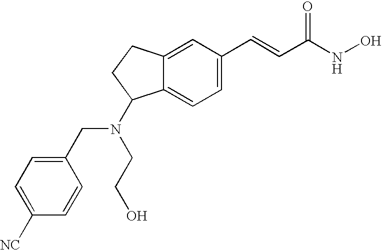 | | | |
| 154 | 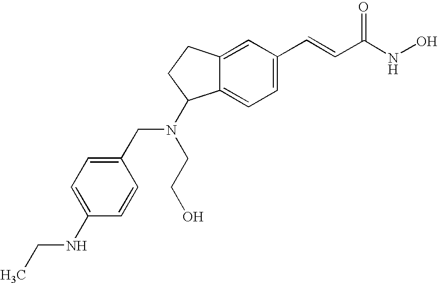 | | | |
| 155 | 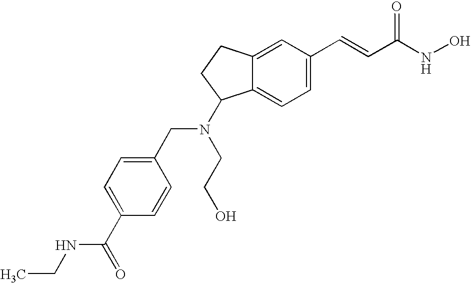 | | | |
| 156 | 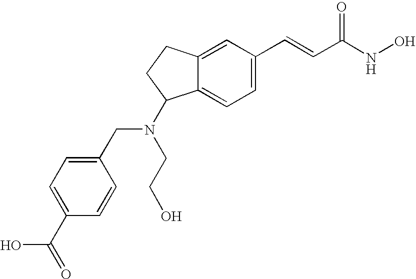 | | | |
| 157 | 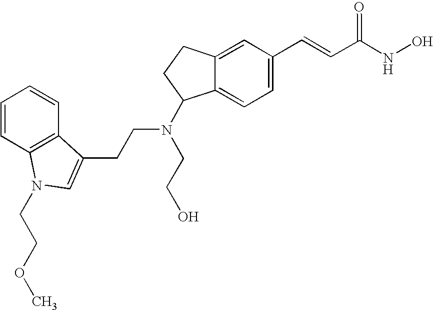 | | | |
| 158 | 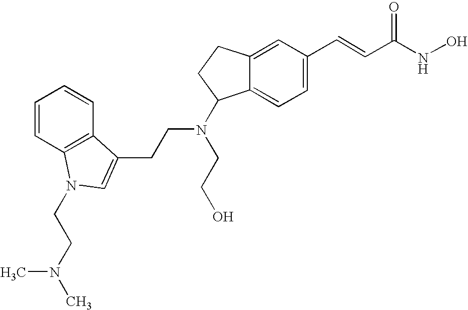 | | | |
| 159 | 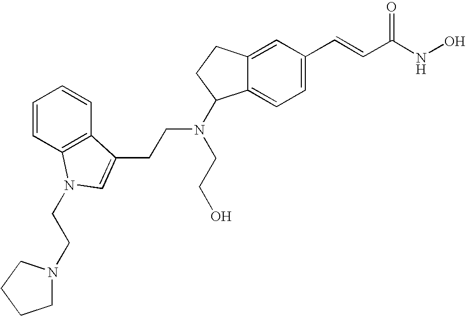 | | | |
| 160 | 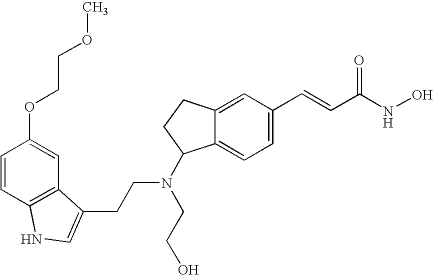 | | | |
| 161 | 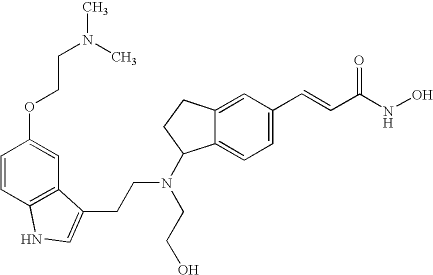 | | | |
| 162 | 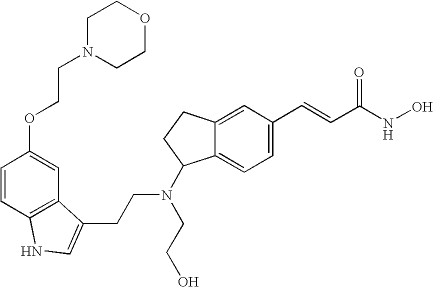 | D, Q37, F38, U1 J37, 162 | 0.98 (A) | 535.0 |
| 163 | 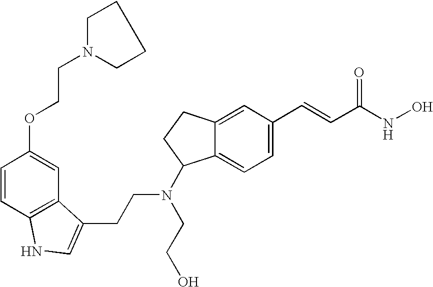 | | | |
| 164 | 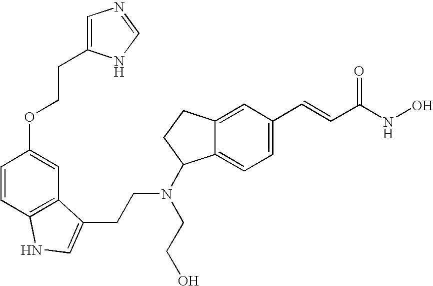 | | | |
| 165 | 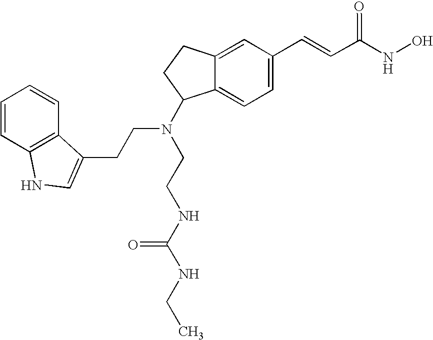 | D, E, F2, T, N4, 165 | 0.98 (A) | 476.1 |
| 166 | 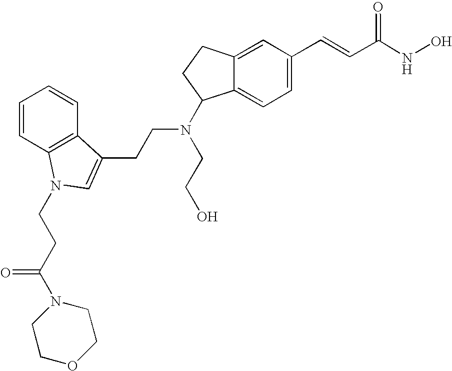 | | | |
| 167 | 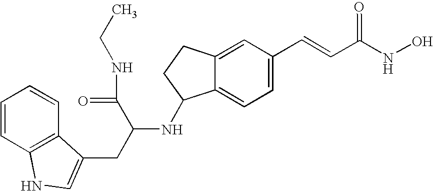 | | | |
| 168 | 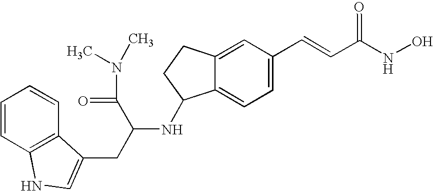 | | | |
| 169 | 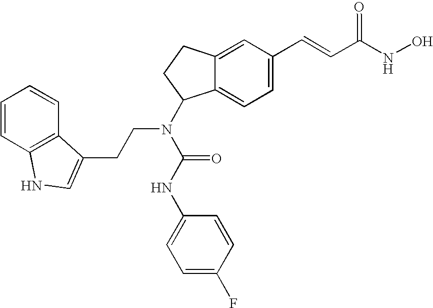 | | | |
| 170 | 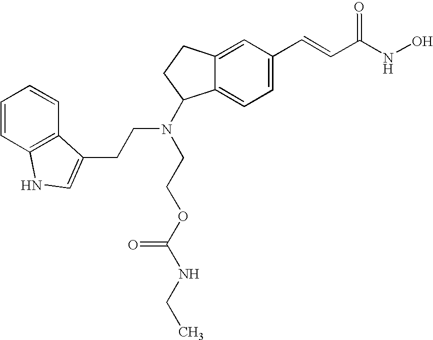 | | | |
| 171 | 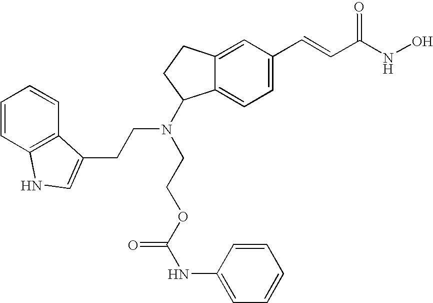 | | | |
| 172 | 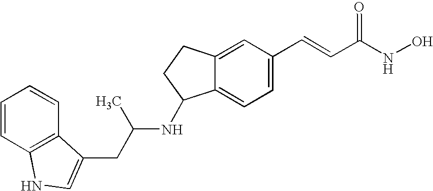 | | | |
| 173 | 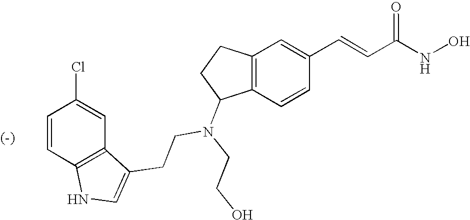 | D, Q5, F10, J11, R45, 173 | 1.67 (A) | 440.1 |
| 174 | 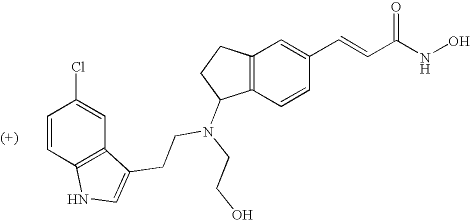 | D, Q5, F10, J11, R46, 174 | 1.81 (A) | 440.1 |
| 175 | 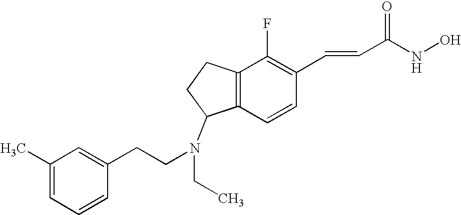 | AD, AE, AF, AG, Q64, F167, D5, 175 | 1.90 (A) | 383.1 |
| 176 | 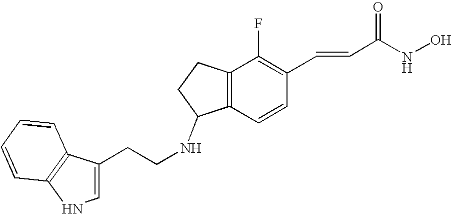 | AD, AE, AF, AG, Q63, D4, 176 | 2.23 (A) | 380.0 |
| 177 | 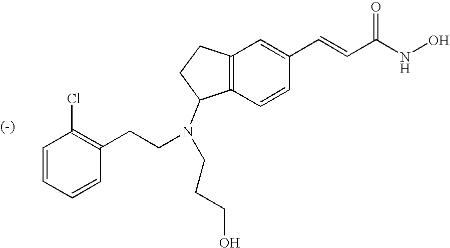 | D, Q18, F21, J20, R5. 177 | 0.99 (A) | 415.0 |
| 178 | 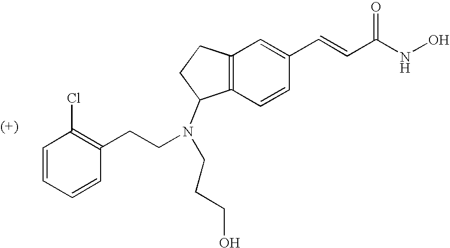 | D, Q18, F21, J20, R6. 178 | 0.96 (A) | 415.0 |
| 179 | 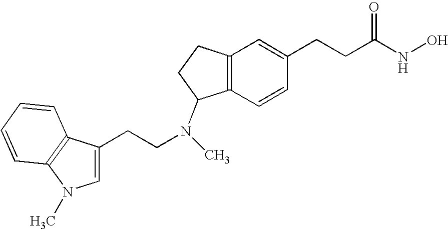 | D, E, V, 179 | 1.95 (A) | 390.0 |
| 180 | 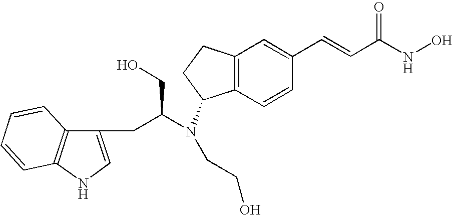 | D, E1, G10, J13, 180 | 1.48 (A) | 436.0 |
| 181 | 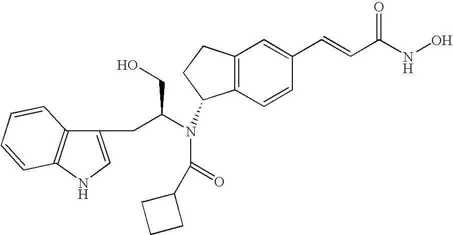 | D, E1, 181 | 2.43 (A) | 474.0 |
| 182 | 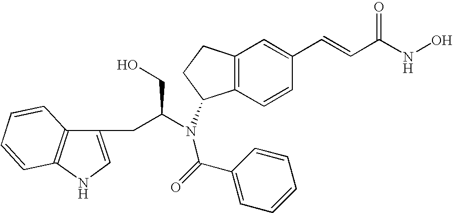 | D, E1, 182 | 2.45 (A) | 496.0 |
| 183 | 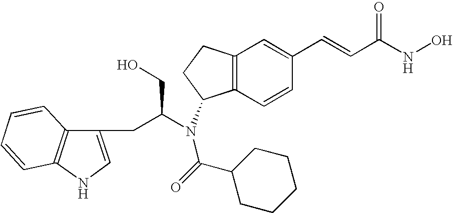 | D, E1, 183 | 2.63 (A) | 502.0 |
| 184 | 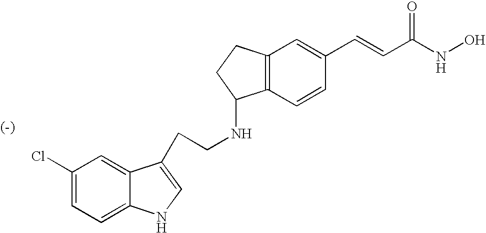 | D, Q5, R47, 184 | 2.07 (A) | 395.9 |
| 185 | 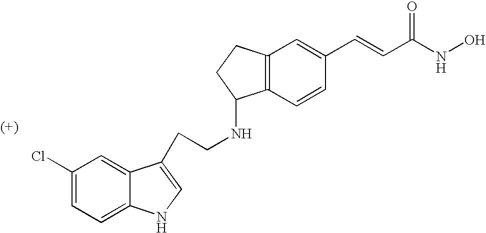 | D, Q5, R48, 185 | 2.10 (A) | 395.5 |
| 186 | 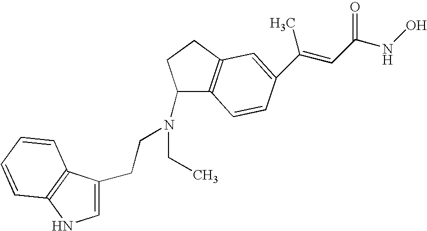 | D3, Q7, F12, 186 | 2.27 (A) | 404.1 |
| 187 | 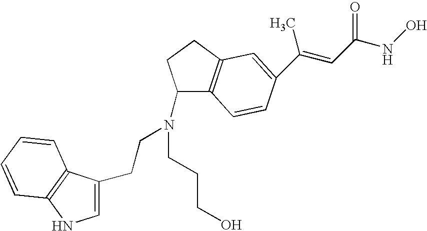 | D3, Q7, G11, J14, 187 | 2.12 (A) | 434.2 |
| 188 | 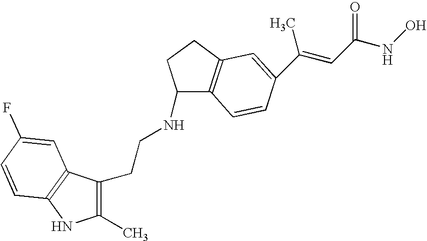 | D3, Q8, 188 | 2.53 (A) | 408.2 |
| 189 | 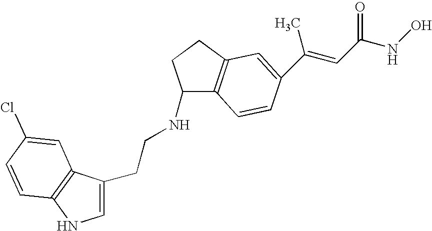 | D3, Q9, 189 | 2.60 (A) | 410.2 |
| 190 | 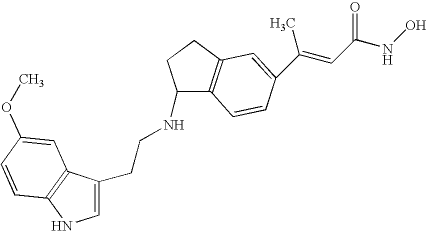 | D3, Q10, 190 | 2.28 (A) | 406.2 |
| 191 | 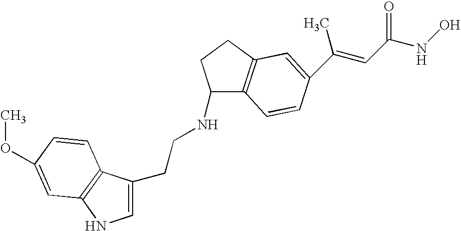 | D3, Q11, 191 | 2.33 (A) | 406.2 |
| 192 | 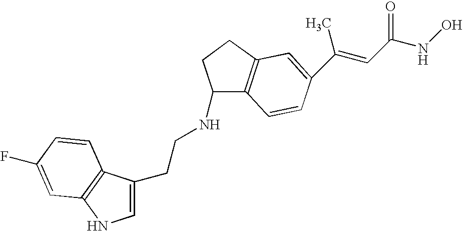 | D3, Q12, 192 | 2.40 (A) | 394.2 |
| 193 | 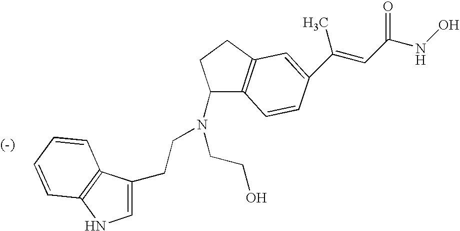 | D3, Q7, G8, J9, R3, 193 | 1.83 (A) | 420.2 |
| 194 | 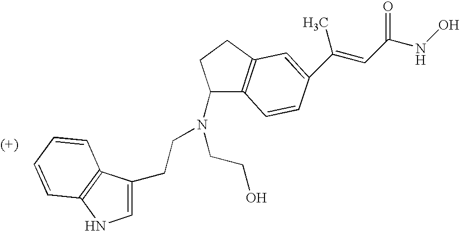 | D3, Q7, G8, J9, R4, 194 | 1.84 (A) | 420.2 |
| 195 | 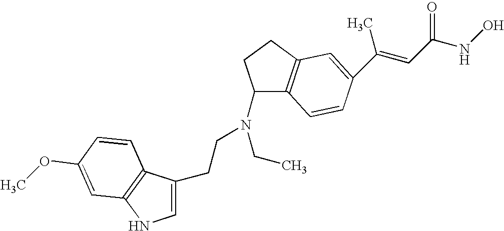 | D3, Q11, F13, 195 | 1.79 (A) | 434.1 |
| 196 | 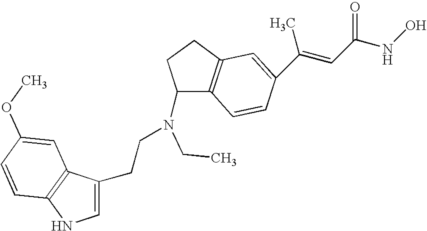 | D3, Q10, F14, 196 | 1.75 (A) | 434.1 |
| 197 | 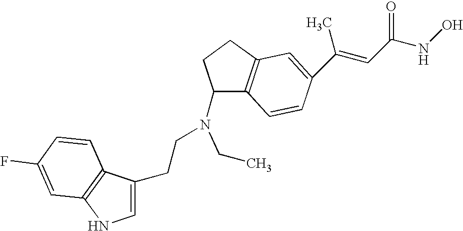 | D3, Q12, F15, 197 | 1.86 (A) | 422.2 |
| 198 | 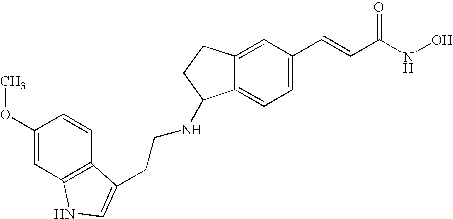 | D, Q13, 198 | 1.91 (A) | 392.0 |
| 199 | 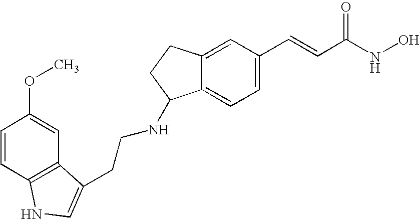 | D, Q4, 199 | 1.78 (A) | 392.0 |
| 200 | 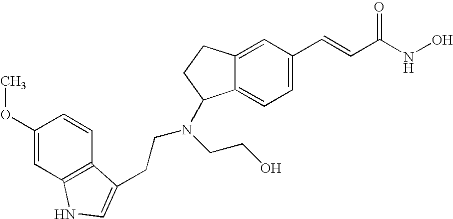 | D, Q13, F16, J15, 200 | 1.44 (A) | 436.0 |
| 201 | 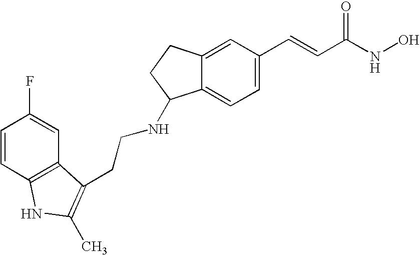 | D, Q6, 201 | 1.91 (A) | 394.0 |
| 202 | 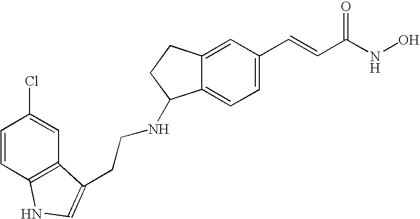 | D, Q5, 202 | 1.98 (A) | 395.9 |
| 203 | 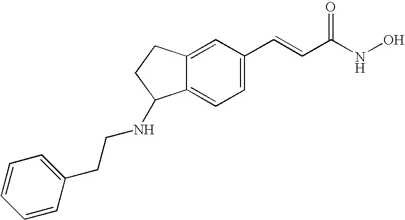 | D, Q14, 203 | 1.62 (A) | 322.9 |
| 204 | 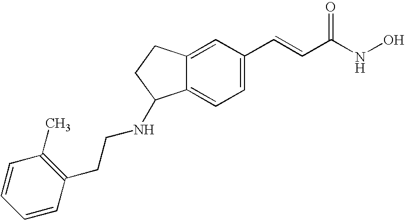 | D, Q15, 204 | 1.84 (A) | 337.1 |
| 205 | 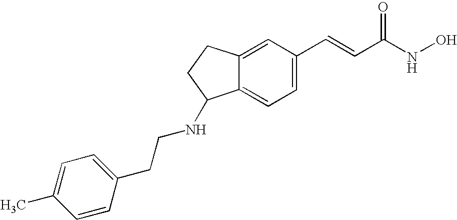 | D, Q16, 205 | 1.87 (A) | 337.1 |
| 206 | 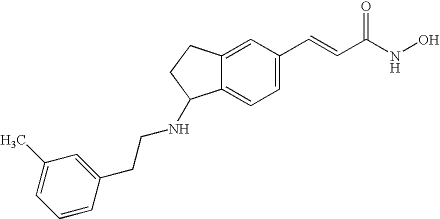 | D, Q17, 206 | 1.87 (A) | 337.1 |
| 207 | 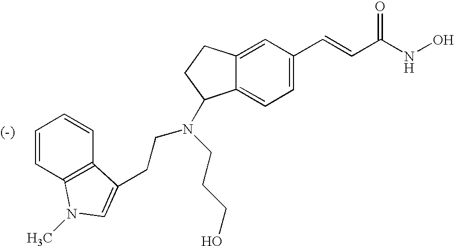 | D, E, G2, V1, J38, R23, 207 | 1.89 (A) | 434.1 |
| 208 | 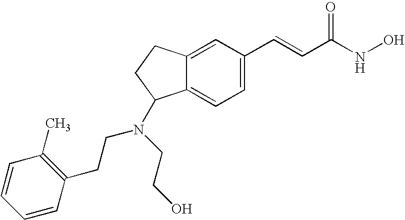 | D, Q15, F17, J16, 208 | 1.81 (A) | 381.1 |
| 209 | 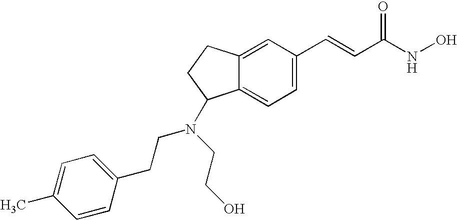 | D, Q16, F18, J17, 209 | 1.85 (A) | 381.1 |
| 210 | 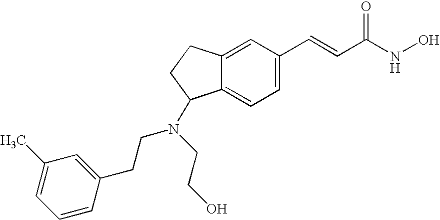 | D, Q17, F19, J18, 210 | 1.83 (A) | 381.1 |
| 211 | 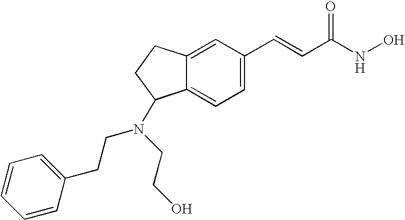 | D, Q14, F20, J19, 211 | 1.64 (A) | 367.0 |
| 212 | 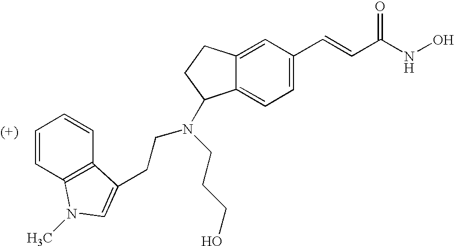 | D, E, G2, V1, J38, R24, 212 | 1.89 (A) | 434.0 |
| 213 | 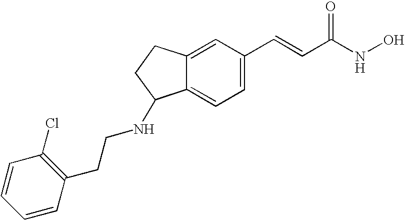 | D, Q18, 213 | 1.83 (A) | 357.0 |
| 214 | 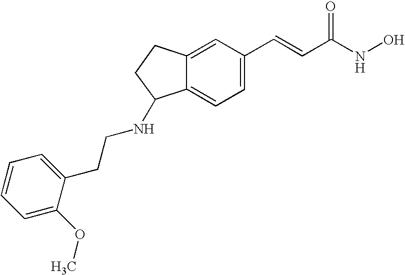 | D, Q19, 214 | 1.76 (A) | 353.0 |
| 215 | 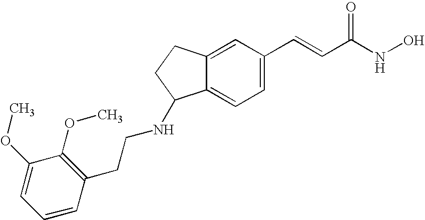 | D, Q20, 215 | 1.75 (A) | 383.1 |
| 216 | 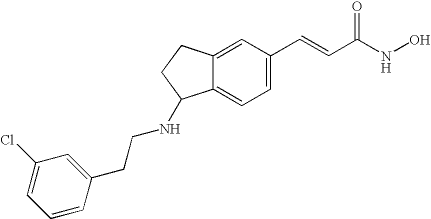 | D, Q21, 216 | 1.87 (A) | 357.0 |
| 217 | 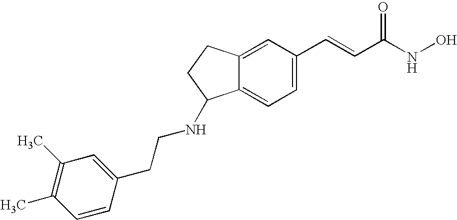 | D, Q22, 217 | 1.98 (A) | 351.1 |
| 218 | 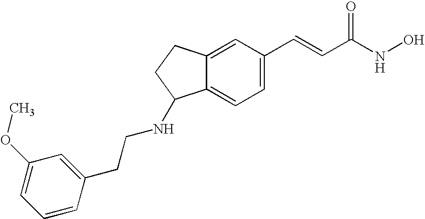 | D, Q23, 218 | 1.69 (A) | 353.0 |
| 219 | 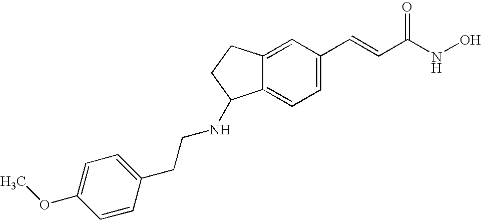 | D, Q24, 219 | 1.69 (A) | 353.0 |
| 220 | 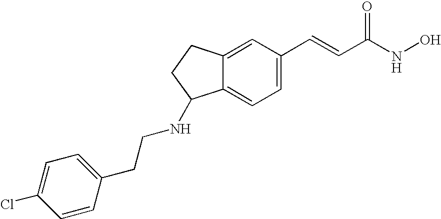 | D, Q25, 220 | 1.90 (A) | 357.0 |
| 221 | 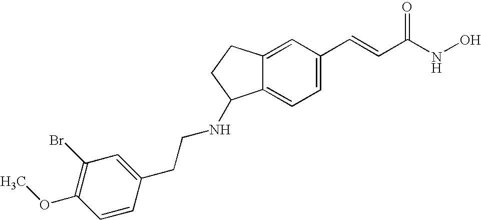 | D, Q26, 221 | 1.92 (A) | 431.0 |
| 222 | 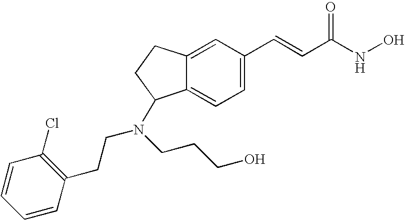 | D, Q18, F21, J20, 222 | 1.79 (A) | 415.0 |
| 223 | 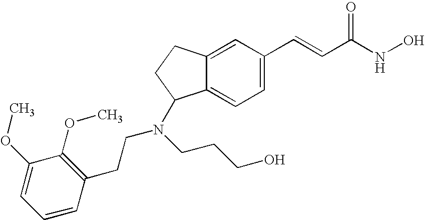 | D, Q20, F22, J21, 223 | 1.72 (A) | 441.1 |
| 224 | 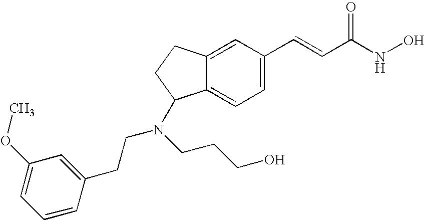 | D, Q23, F23, J22, 224 | 1.63 (A) | 411.0 |
| 225 | 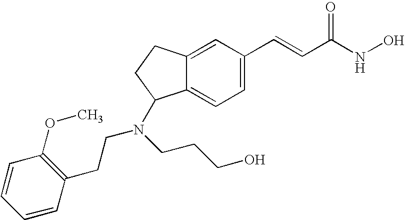 | D, Q19, F24, J23, 225 | 1.76 (A) | 411.0 |
| 226 | 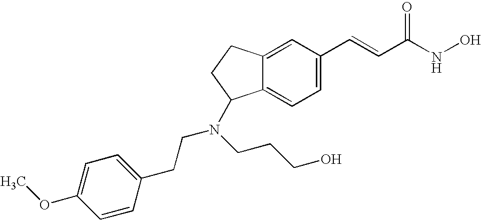 | D, Q24, F25, J24, 226 | 1.64 (A) | 411.0 |
| 227 | 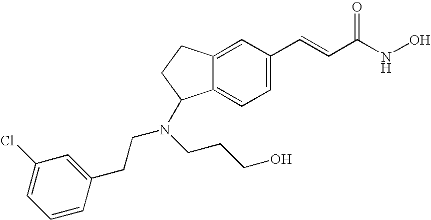 | D, Q21, F26, J25, 227 | 1.83 (A) | 415.0 |
| 228 | 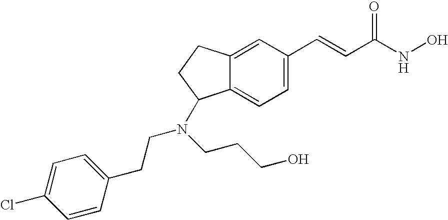 | D, Q25, F27, J26, 228 | 1.85 (A) | 415.0 |
| 229 | 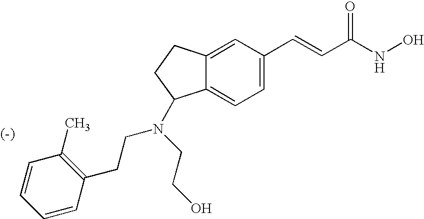 | D, Q15, F17, J16, R25, 229 | 1.92 (A) | 381.0 |
| 230 | 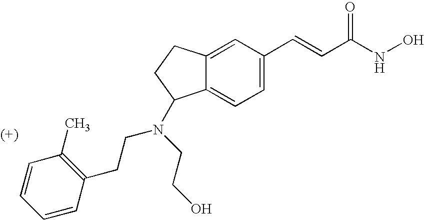 | D, Q15, F17, J16, R26, 230 | 1.92 (A) | 381.0 |
| 231 | 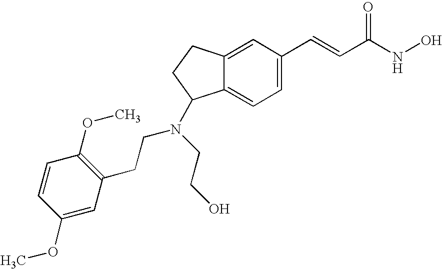 | D, Q27, F28, J27, 231 | 1.75 (A) | 427.0 |
| 232 | 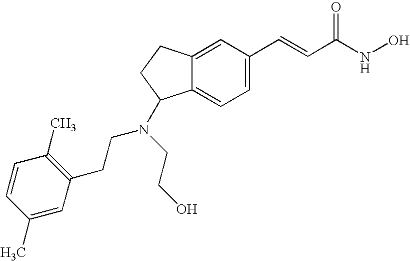 | D, Q28, F29, J28, 232 | 1.90 (A) | 395.0 |
| 233 | 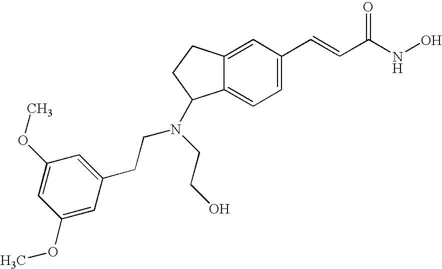 | D, Q29, F30, J29, 233 | 1.68 (A) | 427.0 |
| 234 | 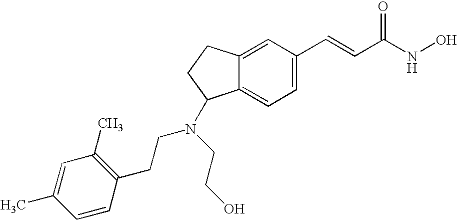 | D, Q30, F31, J30, 234 | 1.91 (A) | 395.0 |
| 235 | 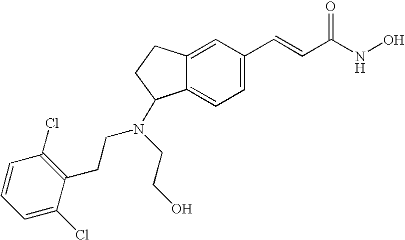 | D, Q31, F32, J31, 235 | 1.86 (A) | 435.0 |
| 236 | 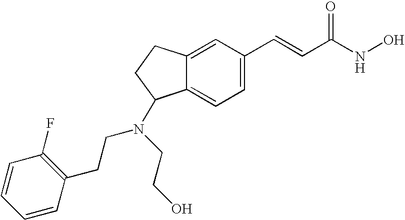 | D, Q32, F33, J32, 236 | 1.60 (A) | 385.0 |
| 237 | 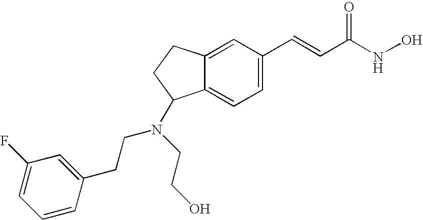 | D, Q33, F34, J33, 237 | 1.59 (A) | 385.0 |
| 238 | 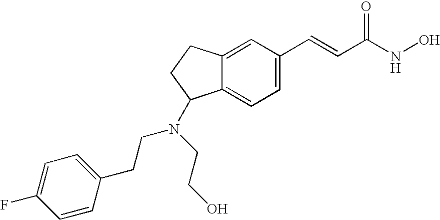 | D, Q34, F35, J34, 238 | 1.61 (A) | 385.0 |
| 239 | 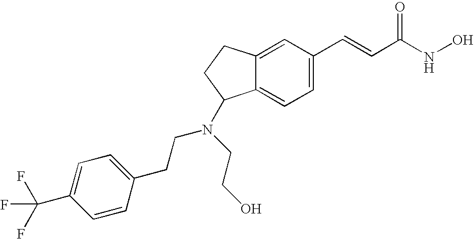 | D, Q35, F36, J35, 239 | 1.94 (A) | 434.9 |
| 240 | 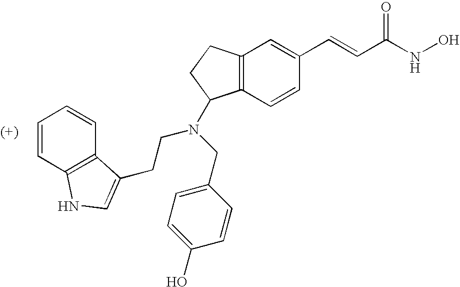 | D, E, F39, R27, 240 | 1.82 (A) | 468.1 |
| 241 | 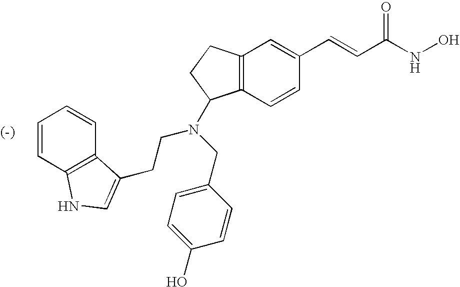 | D, E, F39, R28, 241 | 1.91 (A) | 468.1 |
| 242 | 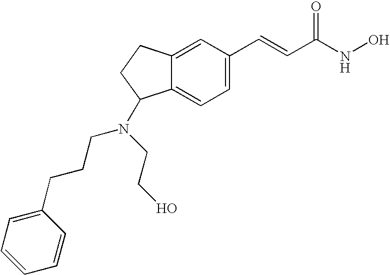 | D, Q36, F37, J36, 242 | 1.73 (A) | 381.0 |
| 243 | 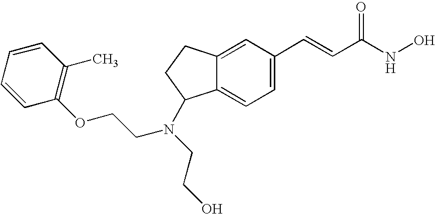 | D, Q38, F168, W1, 243 | 1.85 (A) | 397.0 |
| 244 | 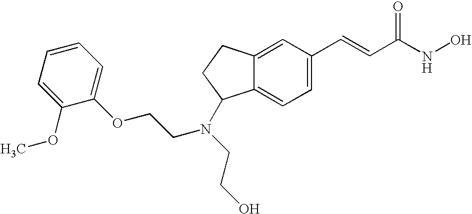 | D, Q38, F168, W2, 244 | 1.84 (A) | 413.0 |
| 245 | 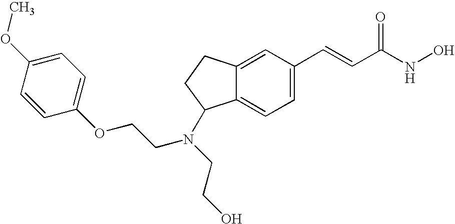 | D, Q38, F168, W3, 245 | 1.94 (A) | 413.0 |
| 246 | 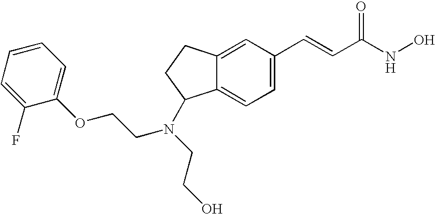 | D, Q38, F168, W4, 246 | 2.23 (A) | 400.9 |
| 247 | 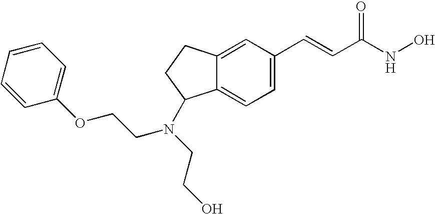 | D, Q38, F168, W, 247 | 1.96 (A) | 383.0 |
| 248 | 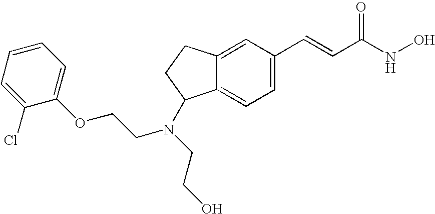 | D, Q38, F168, W5, 248 | 2.33 (A) | 417.0 |
| 249 | 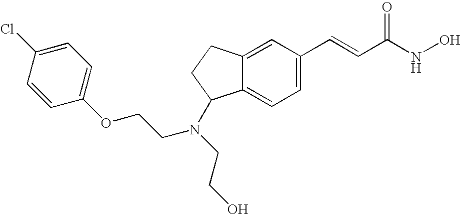 | D, Q38, F168, W6, 249 | 2.39 (A) | 417.0 |
| 250 | 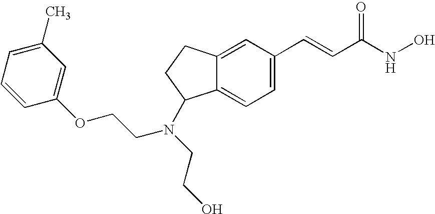 | D, Q38, F168, W7, 250 | 2.26 (A) | 397.0 |
| 251 | 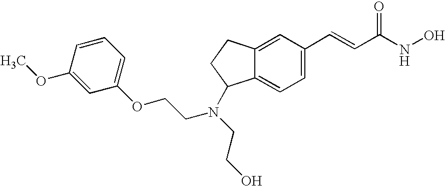 | D, Q38, F168, W8, 251 | 1.99 (A) | 413.0 |
| 252 | 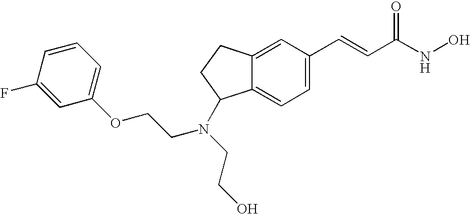 | D, Q38, F168, W9, 252 | 2.30 (A) | 400.9 |
| 253 | 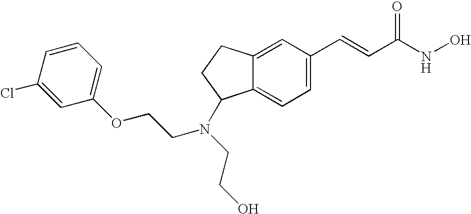 | D, Q38, F168, W10, 253 | 2.30 (A) | 417.0 |
| 254 | 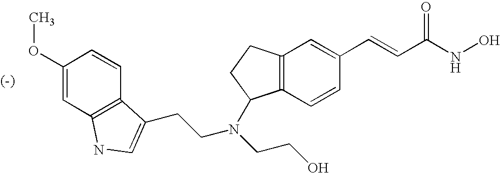 | D, Q13, F16, J15, R31, 254 | 1.30 (A) | 436.0 |
| 255 | 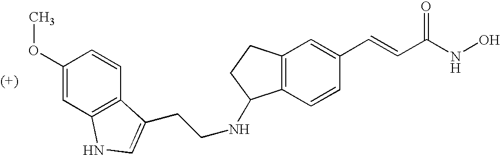 | D, Q13, R29, 255 | 1.83 (A) | 392.0 |
| 256 | 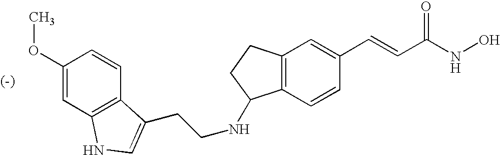 | D, Q13, R30, 256 | 1.85 (A) | 392.0 |
| 257 | 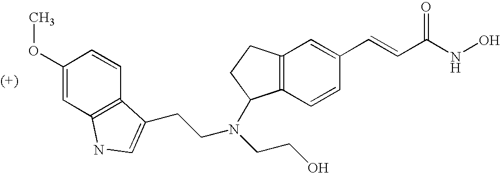 | D, Q13, F16, J15, R32, 257 | 1.23 (A) | 436.0 |
| 258 | 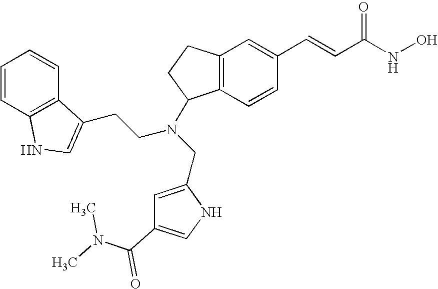 | D, E, F98, X7, 258 | 1.19 (A) | 511.9 |
| 259 | 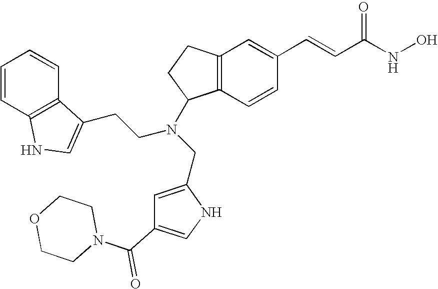 | D, E, F98, X8, 259 | 1.36 (A) | 554.0 |
| 260 | 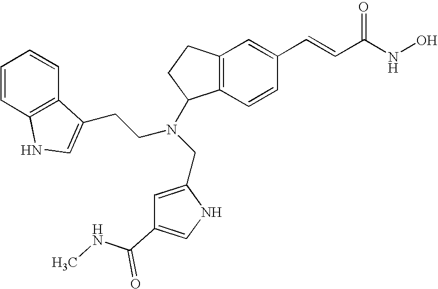 | D, E, F98, X9, 260 | 0.99 (A) | 497.9 |
| 261 | 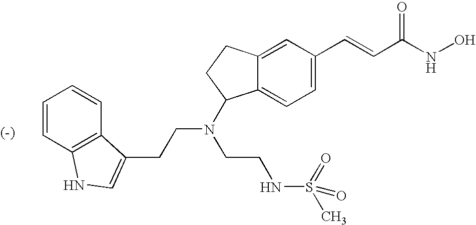 | D, E, F2, T, O2, R33, 261 | 0.97 (A) | 483.1 |
| 262 | 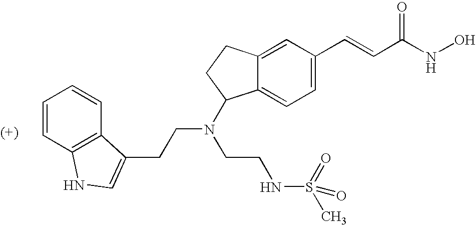 | D, E, F2, T, O2, R34, 262 | 0.97 (A) | 483.1 |
| 263 | 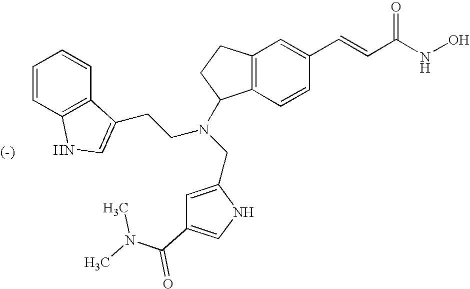 | D, E, F98, X7, R35, 263 | 0.99 (A) | 512.0 |
| 264 | 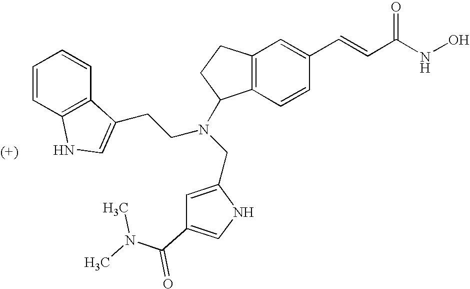 | D, E, F98, X7, R36, 264 | 0.95 (A) | 512.0 |
| 265 | 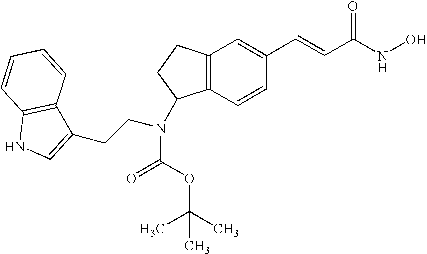 | D, E, A3, 265 | 3.06 (A) | 462.0 |
| 266 | 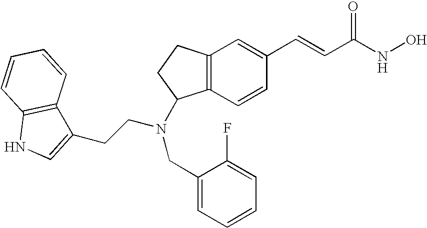 | D, E, F40, 266 | 2.13 (A) | 470.1 |
| 267 | 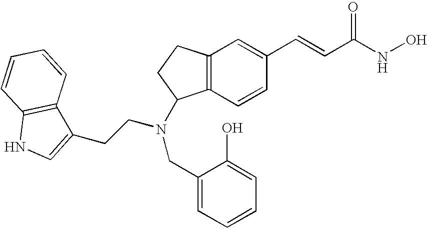 | D, E, F41, 267 | 2.20 (A) | 468.1 |
| 268 | 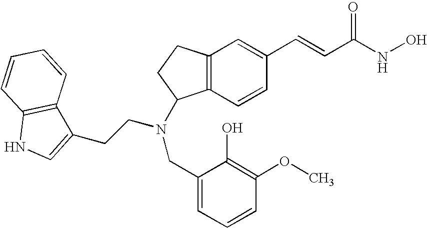 | D, E, F42, 268 | 2.11 (A) | 498.1 |
| 269 | 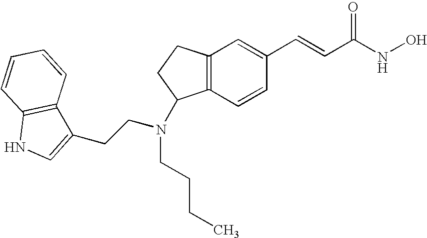 | D, E, F43, 269 | 2.04 (A) | 418.1 |
| 270 | 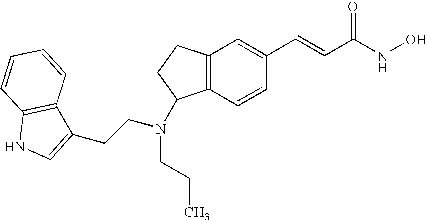 | D, E, F44, 270 | 2.02 (A) | 404.0 |
| 271 | 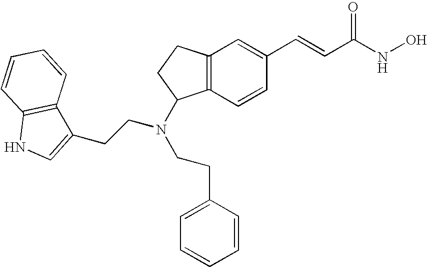 | D, E, F45, 271 | 2.30 (A) | 466.1 |
| 272 | 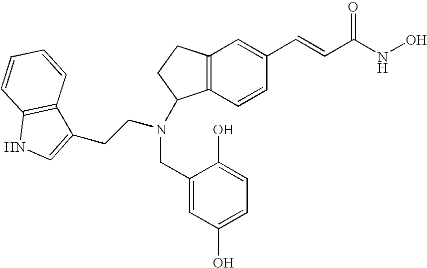 | D, E, F46, 272 | 1.95 (A) | 484.1 |
| 273 | 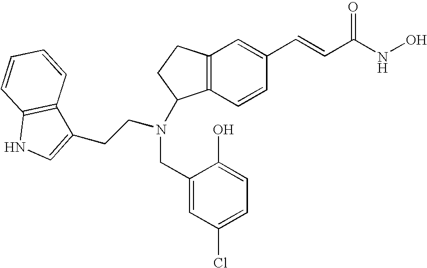 | D, E, F47, 273 | 2.35 (A) | 502.1 |
| 274 | 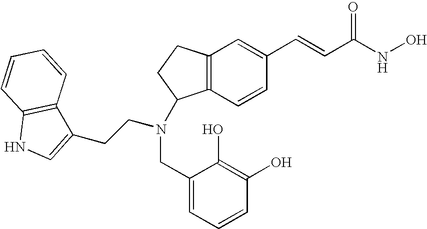 | D, E, F48, 274 | 1.92 (A) | 484.1 |
| 275 | 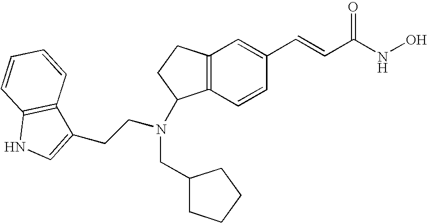 | D, E, F49, 275 | 2.16 (A) | 444.2 |
| 276 | 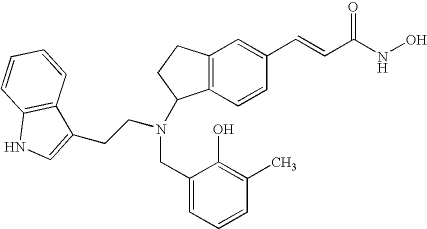 | D, E, F50, 276 | 2.16 (A) | 482.3 |
| 277 | 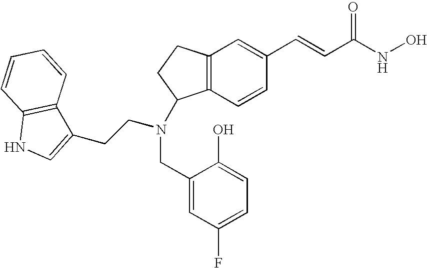 | D, E, F51, 277 | 2.08 (A) | 486.2 |
| 278 | 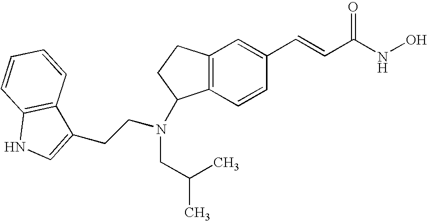 | D, E, F52, 278 | 2.11 (A) | 418.1 |
| 279 | 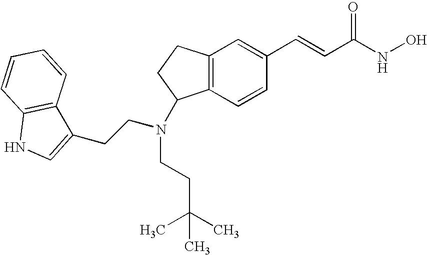 | D, E, F53, 279 | 2.39 (A) | 446.1 |
| 280 | 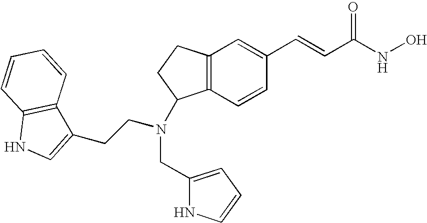 | D, E, F54, 280 | 2.14 (A) | 440.9 |
| 281 | 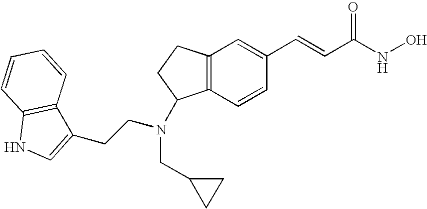 | D, E, F55, 281 | 1.91 (A) | 416.1 |
| 282 | 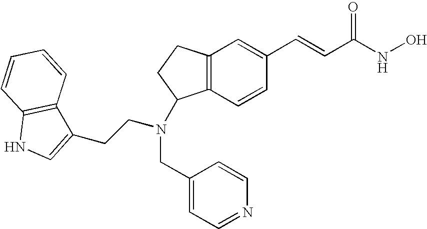 | D, E, F56, 282 | 2.09 (A) | 453.1 |
| 283 | 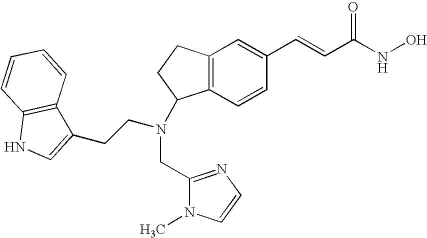 | D, E, F57, 283 | 2.22 (A) | 456.1 |

| | | | HPLC RT | |
|---|---|---|---|---|
| Compound | | Synthetic | (min) | |
| Example | Structure | sequence | (method) | M + H |
| 284 | 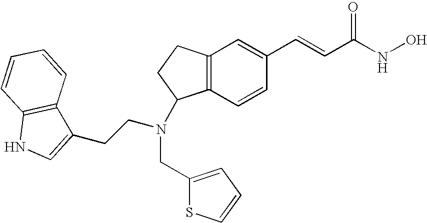 | D, E, F58, 284 | 2.04 (A) | 458.0 |
| 285 | 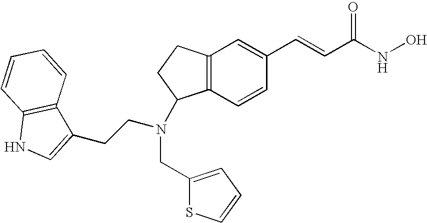 | D, E, F59, 284 | 1.95 (A) | 442.0 |
| 286 | 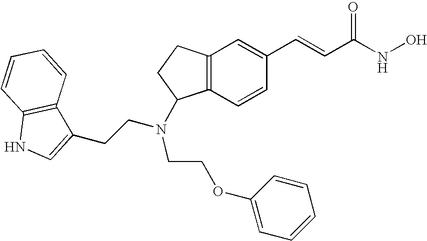 | D, E, G, J, U2, 286 | 2.19 (A) | 482.1 |
| 287 | 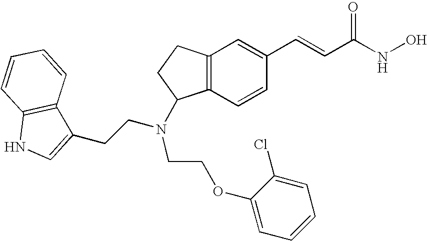 | D, E, G, J, U, 287 | 2.25 (A) | 516.2 |
| 288 | 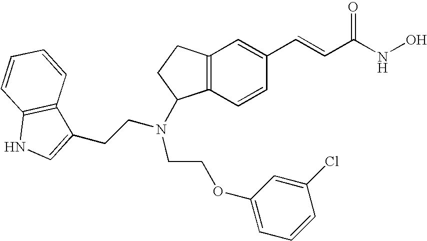 | D, E, G, J, U3, 288 | 2.31 (A) | 516.2 |
| 289 | 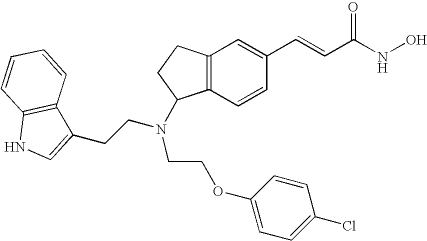 | D, E, G, J, U4, 289 | 2.32 (A) | 516.1 |
| 290 | 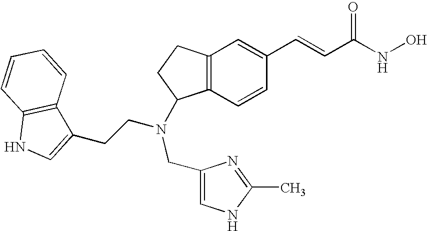 | D, E, F60, 290 | 1.67 (A) | 456.1 |
| 291 | 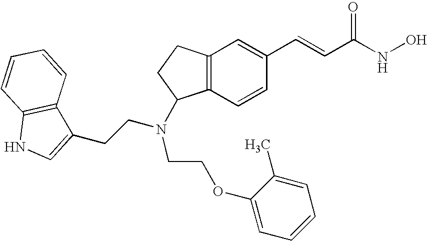 | D, E, G, J, U5, 291 | 2.46 (A) | 496.1 |
| 292 | 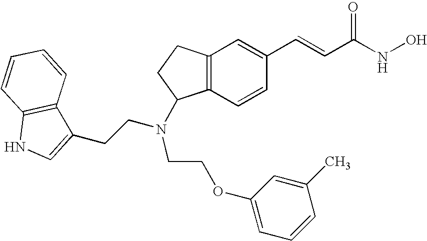 | D, E, G, J, U6, 292 | 2.28 (A) | 496.1 |
| 293 | 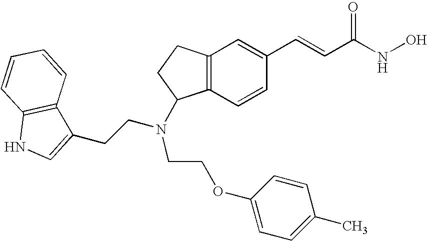 | D, E, G, J, U7, 293 | 2.28 (A) | 496.1 |
| 294 | 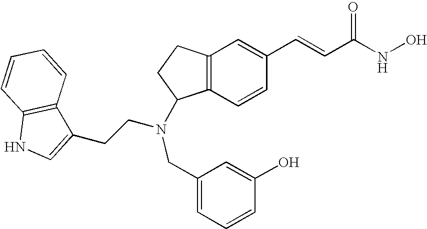 | D, E, F61, 294 | 1.96 (A) | 468.0 |
| 295 | 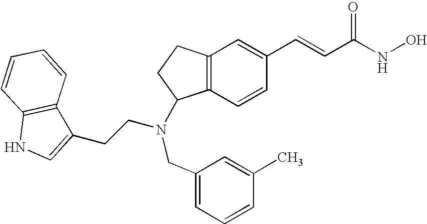 | D, E, F62, 295 | 2.22 (A) | 466.1 |
| 296 | 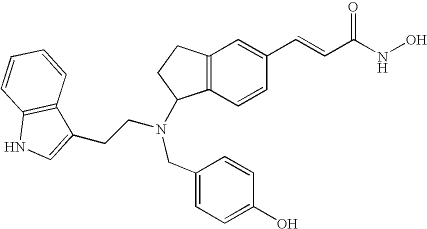 | D, E, F39, 296 | 1.94 (A) | 468.0 |
| 297 | 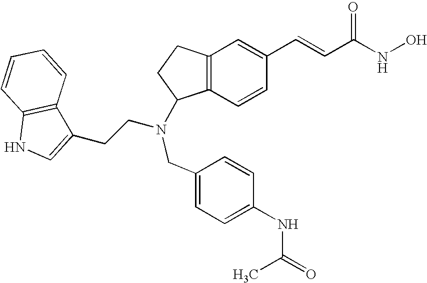 | D, E, F63, 297 | 2.02 (A) | 509.1 |
| 298 | 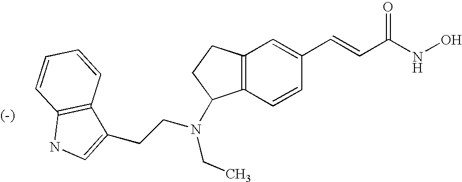 | D, E, F6, R7, 298 | 1.68 (A) | 390.0 |
| 299 | 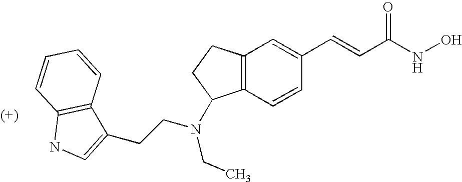 | D, E, F6, R8, 299 | 1.70 (A) | 390.0 |
| 300 | 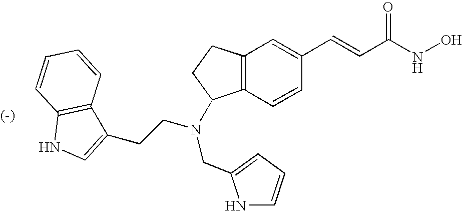 | D, E, F54, R9, 300 | 2.00 (A) | 440.9 |
| 301 | 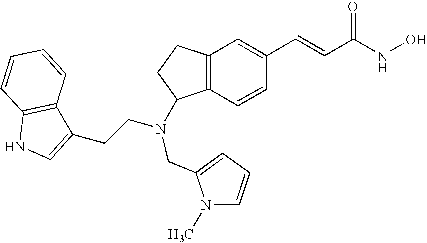 | D, E, F64, 301 | 2.02 (A) | 455.0 |
| 302 | 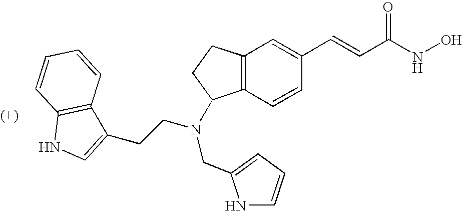 | D, E, F54, R10, 302 | 2.00 (A) | 440.9 |
| 303 | 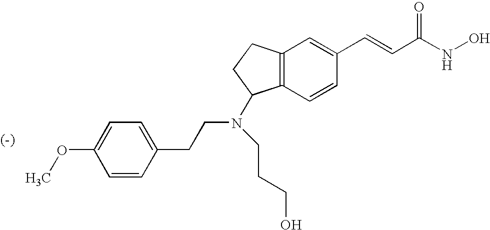 | D, Q24, F25, J24, R11, 303 | 1.73 (A) | 411.1 |
| 304 | 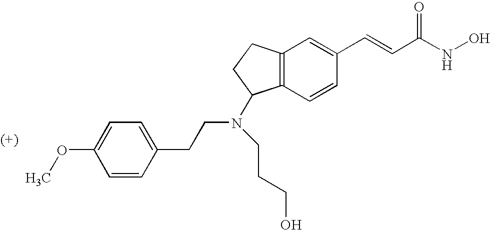 | D, Q24, F25, J24, R12, 304 | 1.74 (A) | 411.1 |
| 305 | 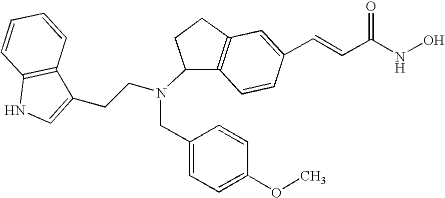 | D, E, F65, 305 | 2.25 (A) | 482.1 |
| 306 | 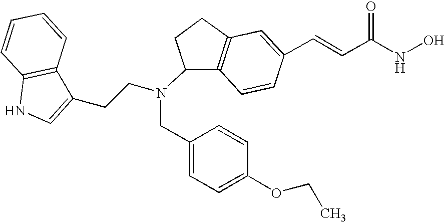 | D, E, F66, 306 | 2.43 (A) | 496.1 |
| 307 | 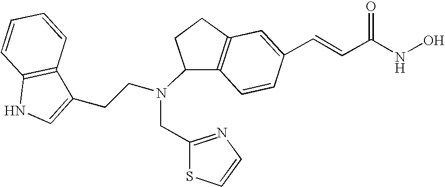 | D, E, F67, 307 | 2.02 (A) | 459.0 |
| 308 | 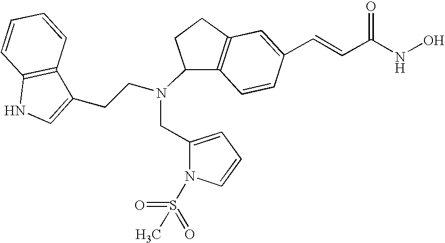 | D, E, F68, 308 | 2.05 (A) | 519.0 |
| 309 | 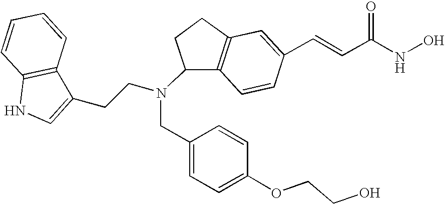 | D, E, F69, 309 | 1.17 (A) | 512.1 |
| 310 | 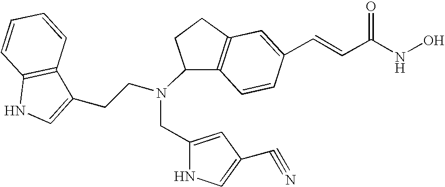 | D, E, F70, 310 | 1.95 (A) | 466.0 |
| 311 | 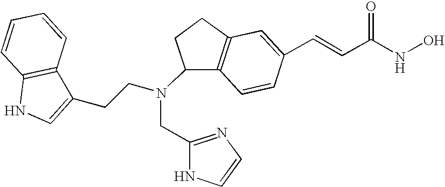 | D, E, F71, 311 | 2.08 (A) | 442.1 |
| 312 | 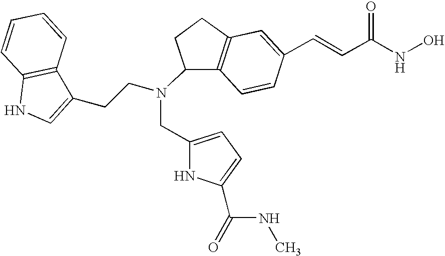 | D, E, F72, X1, 312 | 1.92 (A) | 498.0 |
| 313 | 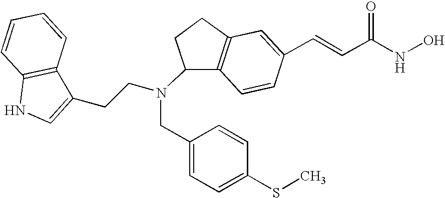 | D, E, F73, 313 | 2.42 (A) | 498.1 |
| 314 | 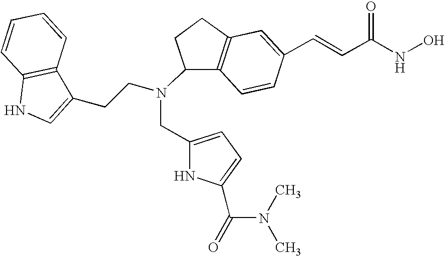 | D, E, F72, X3, 314 | 1.19 (A) | 512.0 |
| 315 | 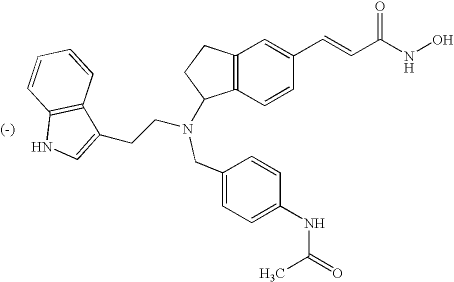 | D, E, F63, R13, 315 | 2.01 (A) | 509.2 |
| 316 | 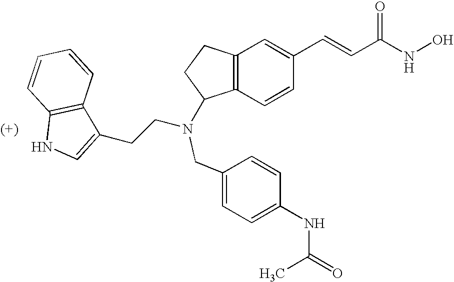 | D, E, F63, R14, 316 | 2.01 (A) | 509.1 |
| 317 | 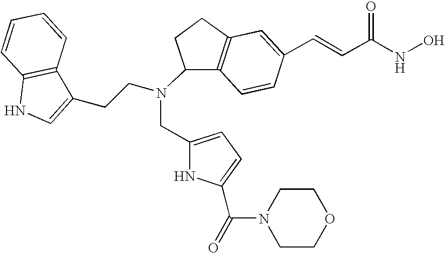 | D, E, F72, X4, 317 | 1.50 (A) | 554.0 |
| 318 | 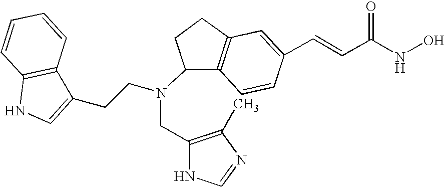 | D, E, F74, 318 | 1.10 (A) | 456.1 |
| 319 | 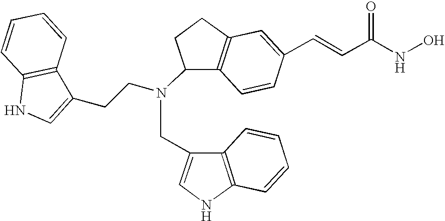 | D, E, F75, 319 | 2.37 (A) | 491.0 |
| 320 | 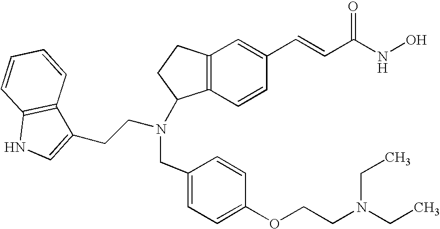 | D, E, F76, 320 | 1.74 (A) | 567.3 |
| 321 | 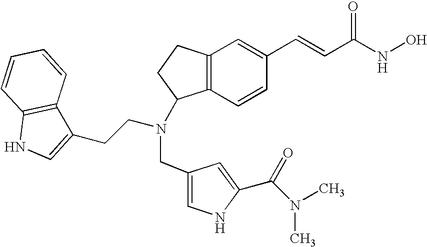 | D, E, F77, X5, 321 | 1.26 (A) | 512.1 |
| 322 | 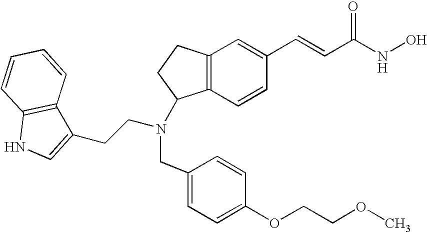 | D, E, F78, 322 | 2.22 (A) | 526.1 |
| 323 | 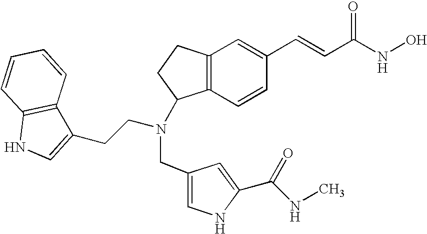 | D, E, F77, X6, 323 | 1.38 (A) | 498.1 |
| 324 | 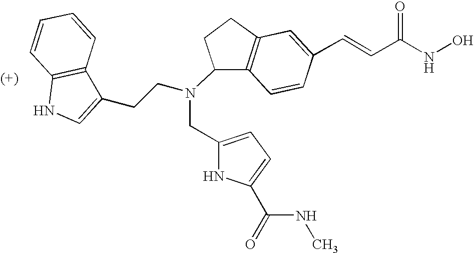 | D, E, F72, X1, R15, 324 | 1.92 (A) | 498.1 |
| 325 | 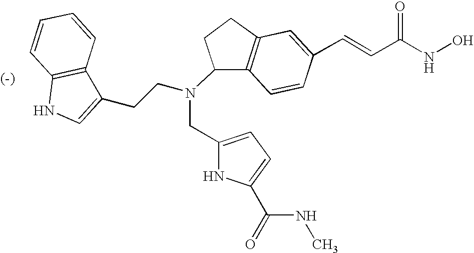 | D, E, F72, X1, R16, 325 | 1.93 (A) | 498.1 |
| 326 | 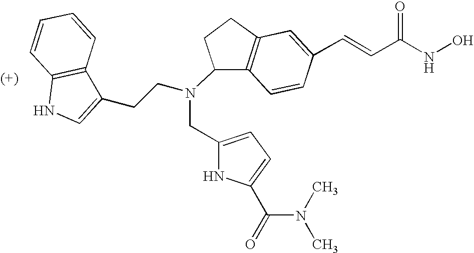 | D, E, F72, X3, R17, 326 | 0.96 (A) | 512.0 |
| 327 | 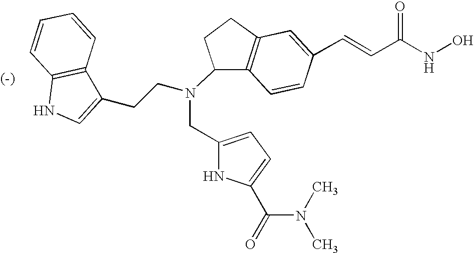 | D, E, F72, X3, R18, 327 | 0.96 (A) | 512.1 |
| 328 | 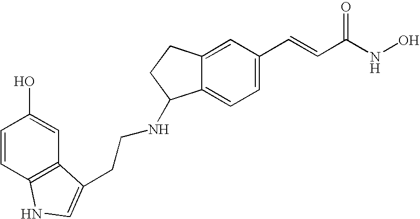 | D, Q37, 328 | 1.02 (A) | 378.6 |
| 329 | 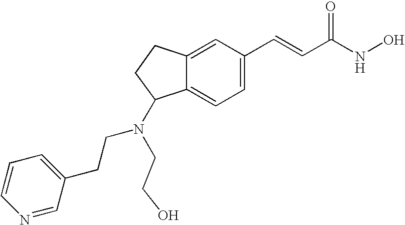 | D, Q1, F79, J40, 329 | 1.02 (A) | 368.1 |
| 330 | 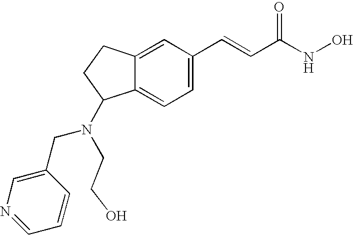 | D, Q, F80, J41, 330 | 1.06 (A) | 354.1 |
| 331 | 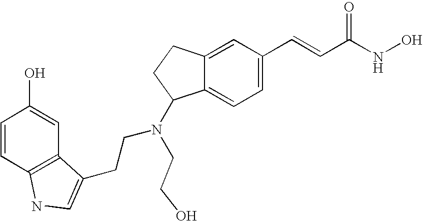 | D, Q37, F38, J42, 331 | 0.87 (A) | 422.0 |
| 332 | 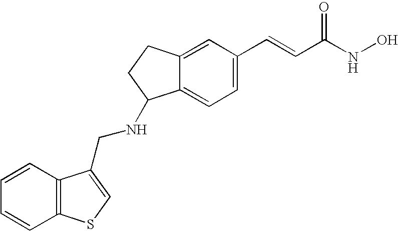 | D, Q40, 332 | 1.88 (A) | 364.9 |
| 333 | 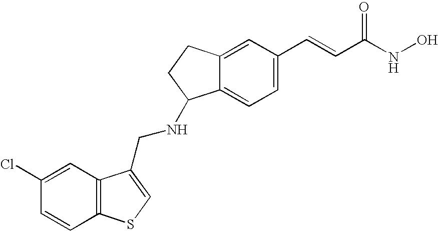 | D, Q41, 333 | 2.07 (A) | 398.8 |
| 334 | 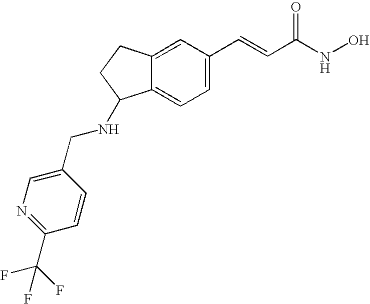 | D, Q42, 334 | 1.16 (A) | 377.7 |
| 335 | 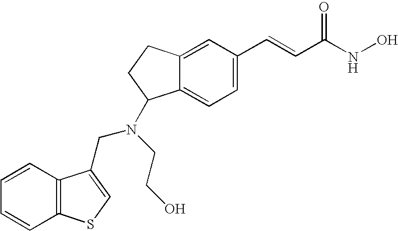 | D, Q40, F82, J43, 335 | 1.98 (A) | 408.9 |
| 336 | 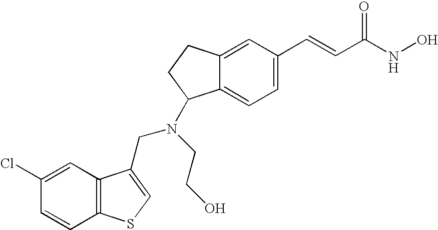 | D, Q41, F83, J44, 336 | 2.12 (A) | 442.9 |
| 337 | 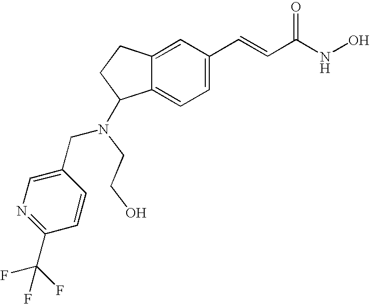 | D, Q42, F84, J45, 337 | 1.30 (A) | 421.9 |
| 338 | 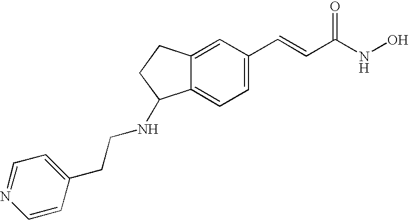 | D, Q43, 338 | 0.78 (A) | 324.0 |
| 339 | 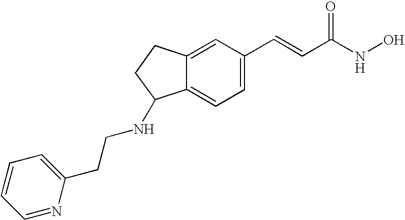 | D, Q44, 339 | 0.81 (A) | 324.0 |
| 340 | 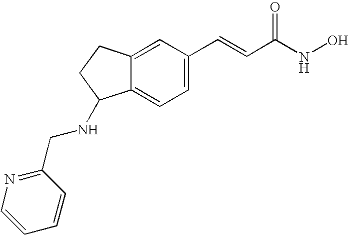 | D, Q45. 340 | 0.87 (A) | 310.0 |
| 341 | 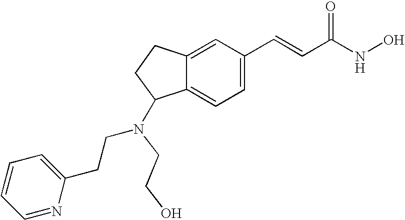 | D, Q44, F85, J46, 341 | 1.08 (A) | 368.0 |
| 342 | 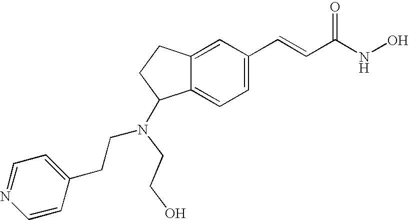 | D, Q43, F86, J47, 342 | 1.03 (A) | 368.0 |
| 343 | 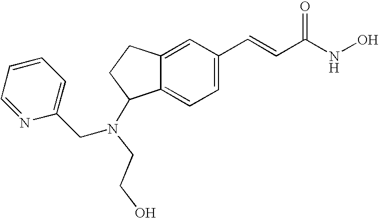 | D, Q45, F87, J48, 343 | 1.10 (A) | 354.0 |
| 344 | 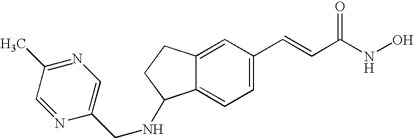 | D, Q46, 344 | 0.86 (A) | 324.9 |
| 345 | 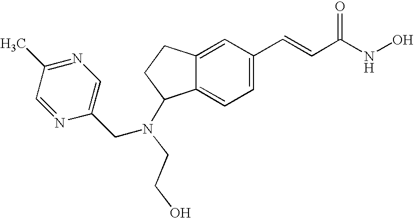 | D, Q46, F88, J49, 345 | 0.99 (A) | 368.9 |
| 346 | 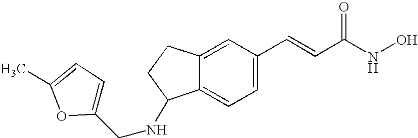 | D, Q47, 346 | 1.19 (A) | 312.9 |
| 347 | 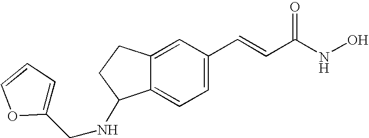 | D, Q48, 347 | 0.96 (A) | 298.9 |
| 348 | 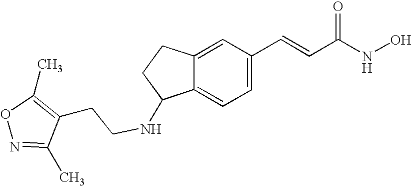 | D, Q49, 348 | 1.03 (A) | 342.0 |
| 349 | 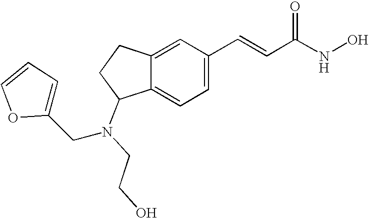 | D, Q48, F89, J50, 349 | 0.28 (A) | 343.0 |
| 350 | 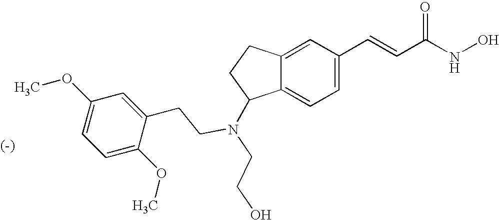 | D, Q27, F28, J27, R39, 350 | 1.61 (A) | 427.0 |
| 351 | 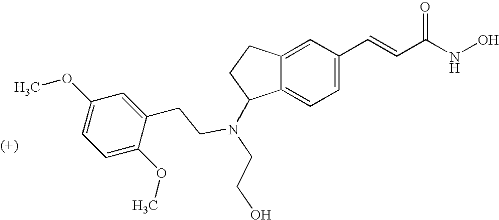 | D, Q27, F28, J27, R40, 351 | 1.58 (A) | 427.0 |
| 352 | 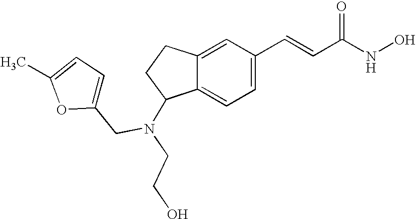 | D, Q47, F90, J51, 352 | 1.12 (A) | 356.9 |
| 353 | 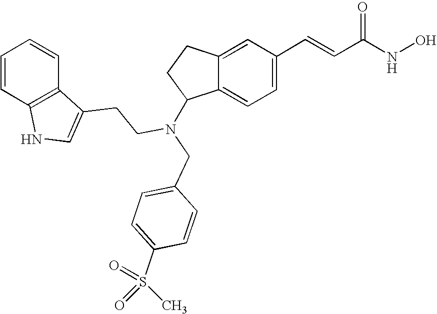 | D, E, F91, 353 | 2.49 (A) | 530.0 |
| 354 | 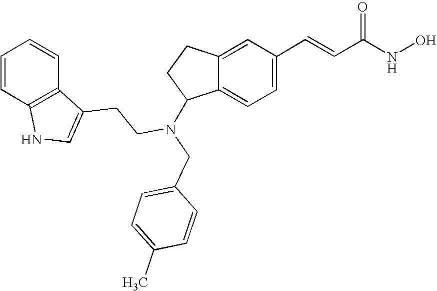 | D, E, F92, 354 | 2.74 (A) | 466.0 |
| 355 | 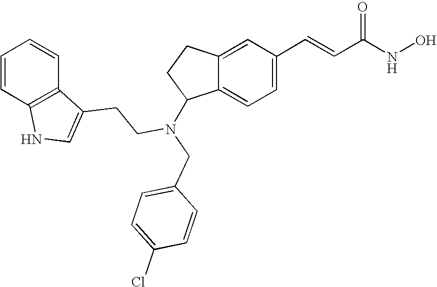 | D, E, F93, 355 | 2.78 (A) | 486.0 |
| 356 | 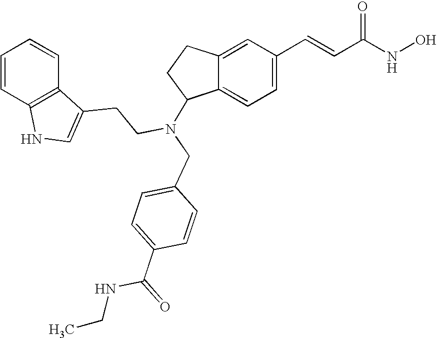 | D, E, F94 (via AH1), 356 | 2.44 (A) | 523.0 |
| 357 | 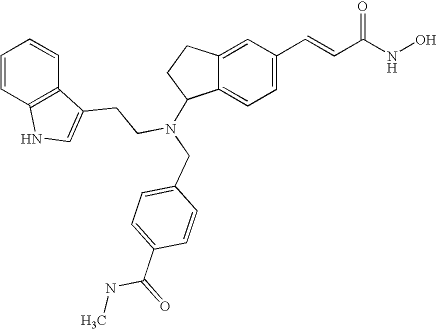 | D, E, F95 (via AH2), 357 | 2.32 (A) | 509.0 |
| 358 | 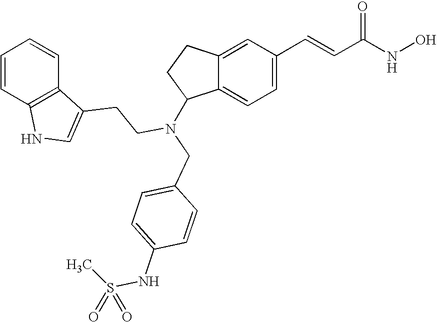 | D, F, F96 (via AH), 358 | 2.07 (A) | 545.1 |
| 359 | 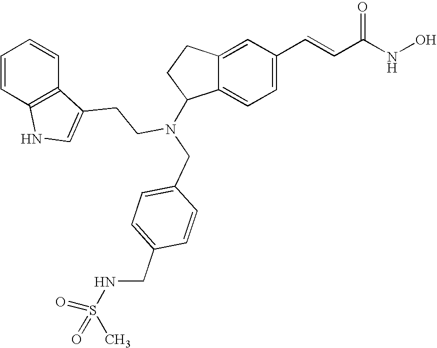 | D, E, F97 (via AH3), 359 | 1.21 (A) | 559.1 |
| 360 | 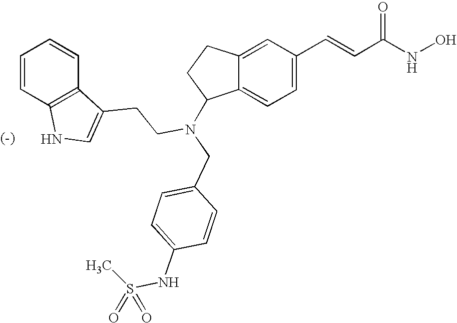 | D, E, F96 (via AH), R41, 360 | 2.08 (A) | 545.1 |
| 361 | 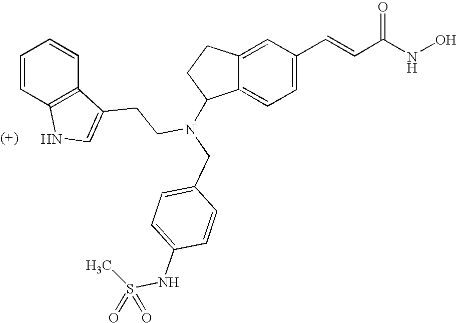 | D, E, F96 (via AH), R42, 361 | 2.07 (A) | 545.1 |
| 362 | 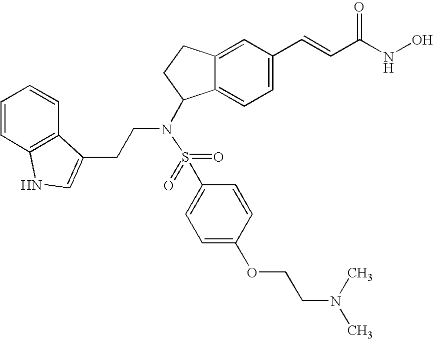 | D, E, O3, Y, Z1, 362 | 2.73 (A) | 589.3 |
| 363 | 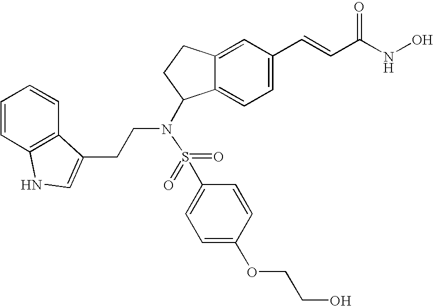 | D, E, O3, Y, Z, J53, 363 | 2.19 (A) | 562.0 |
| 364 | 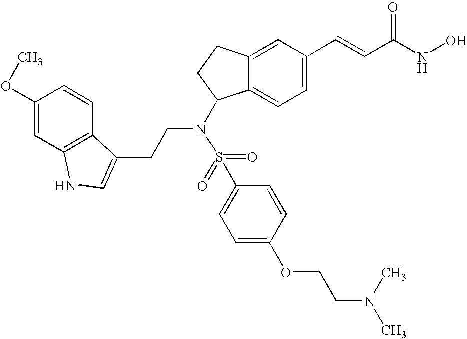 | | | |
| 365 | 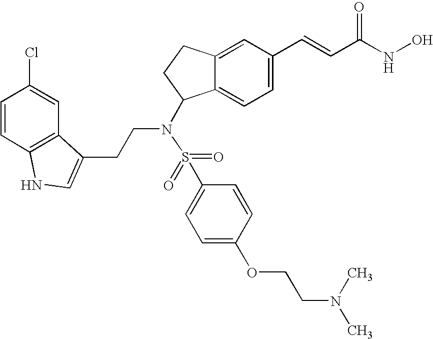 | | | |
| 366 | 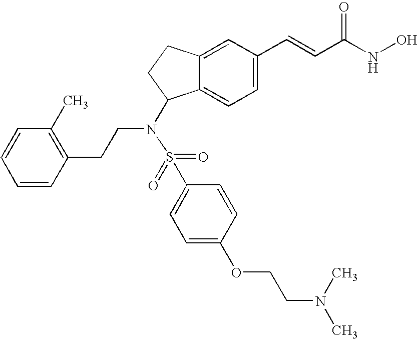 | | | |
| 367 | 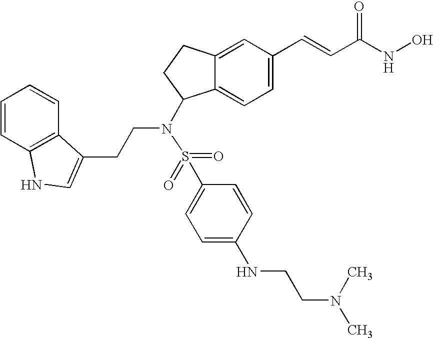 | | | |
| 368 | 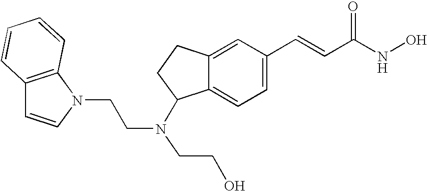 | D, Q50, F100, J54, 368 | 1.92 (A) | 406.1 |
| 369 | 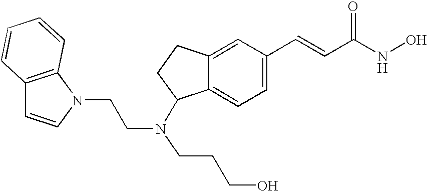 | | | |
| 370 | 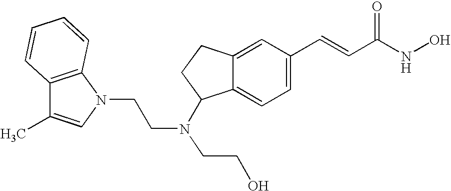 | D, Q51, F101, J55, 370 | 1.67 (A) | 420.1 |
| 371 | 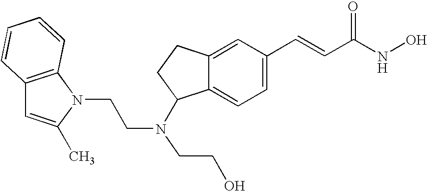 | | | |
| 372 | 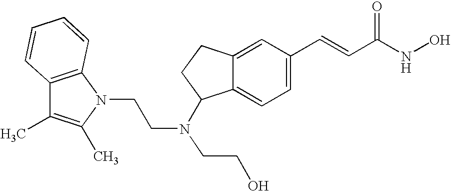 | | | |
| 373 | 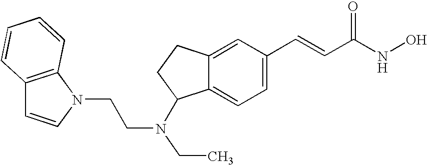 | | | |
| 374 | 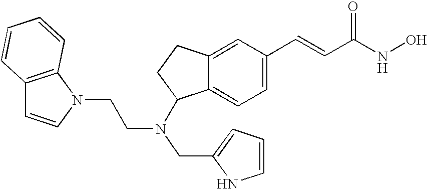 | | | |
| 375 | 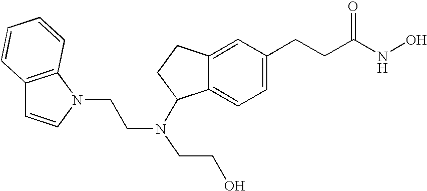 | | | |
| 376 | 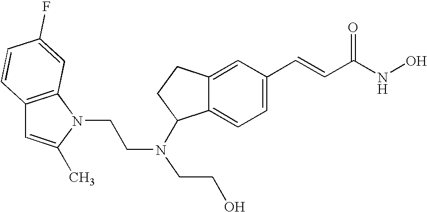 | | | |
| 377 | 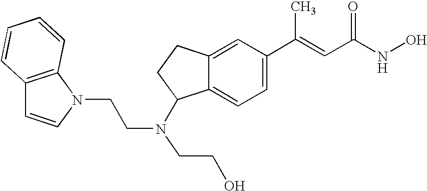 | | | |
| 378 | 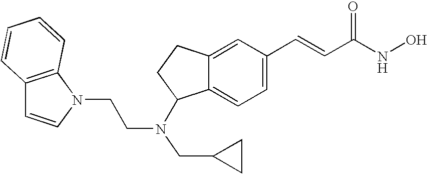 | | | |
| 379 | 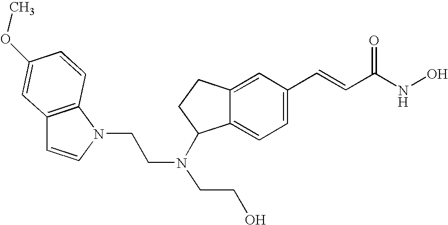 | D, Q52, F102, J56, 379 | 1.03 (A) | 436.0 |
| 380 | 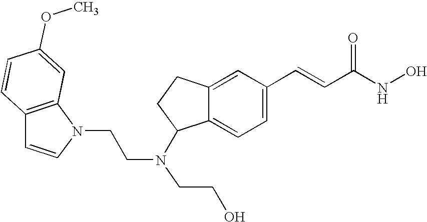 | | | |
| 381 | 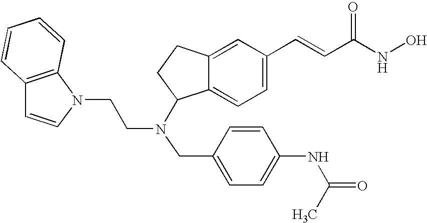 | | | |
| 382 | 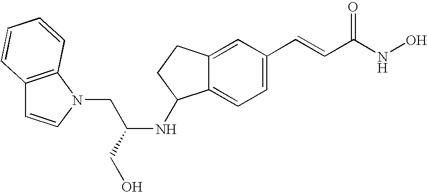 | | | |
| 383 | 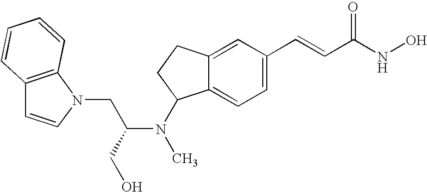 | | | |
| 384 | 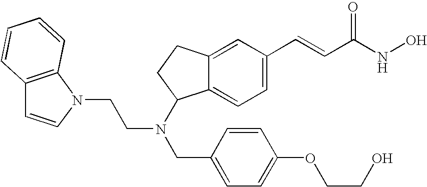 | | | |
| 385 | 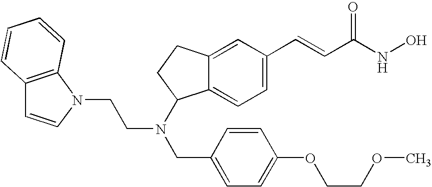 | | | |
| 386 | 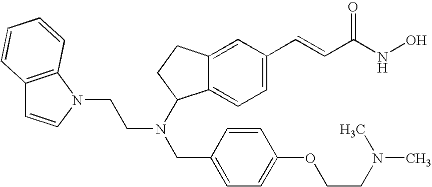 | | | |
| 387 | 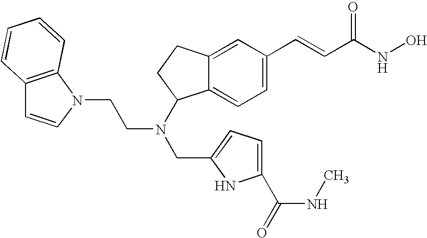 | | | |
| 388 | 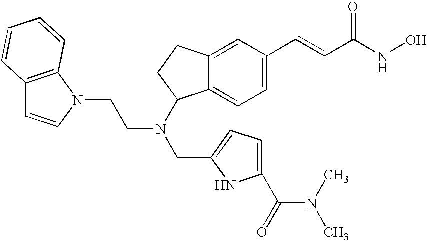 | | | |
| 389 | 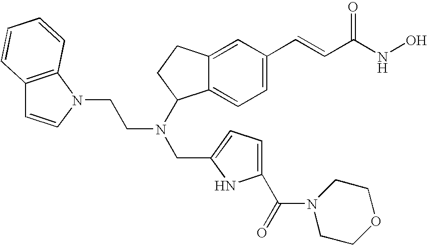 | | | |
| 390 | 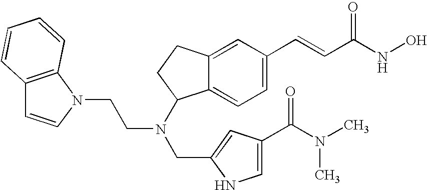 | | | |
| 391 | 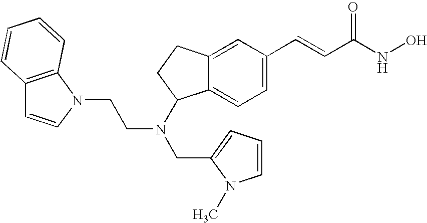 | | | |
| 392 | 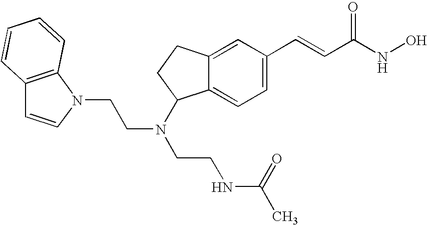 | | | |
| 393 | 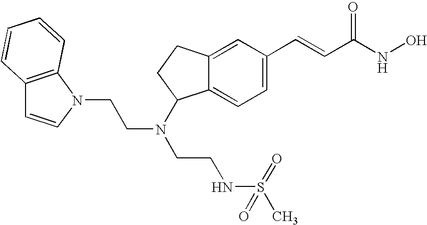 | | | |
| 394 | 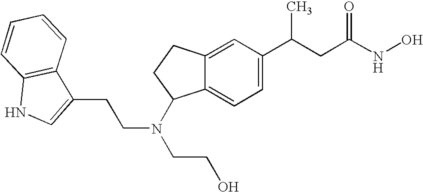 | | | |
| 395 | 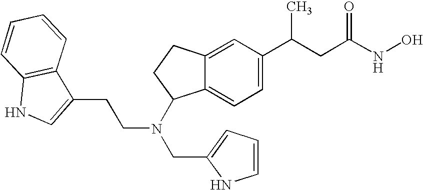 | | | |
| 396 | 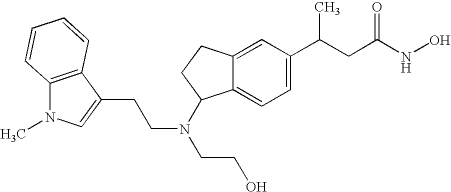 | | | |
| 397 | 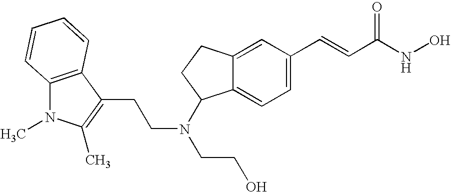 | | | |
| 398 | 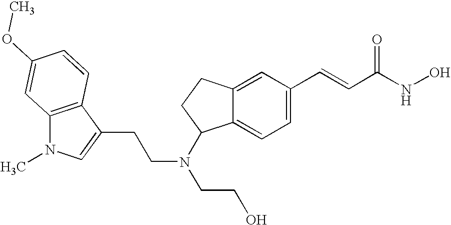 | | | |
| 399 | 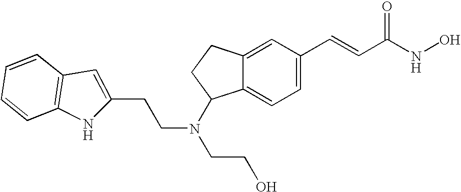 | | | |
| 400 | 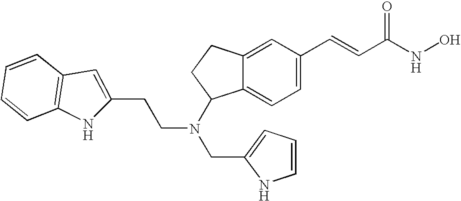 | | | |
| 401 | 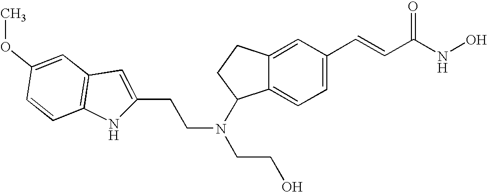 | | | |
| 402 | 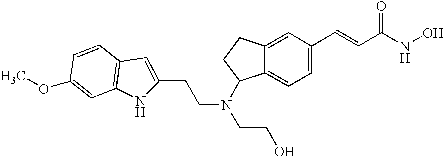 | | | |
| 403 | 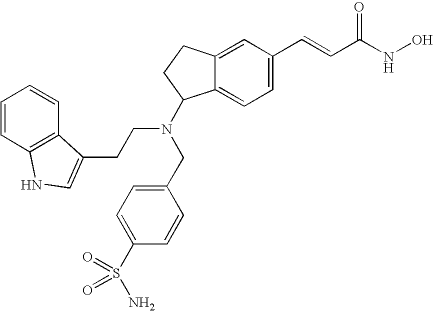 | D, E, F103 (AH4), 403 | 2.02 (A) | 531.1 |
| 404 | 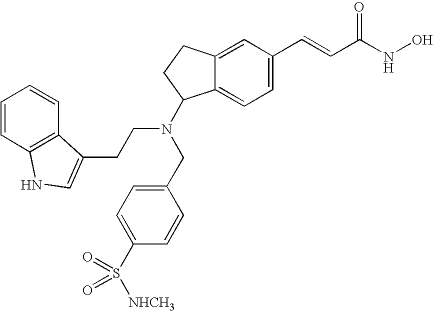 | | | |
| 405 | 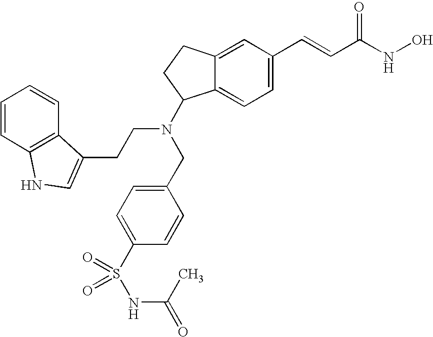 | | | |
| 406 | 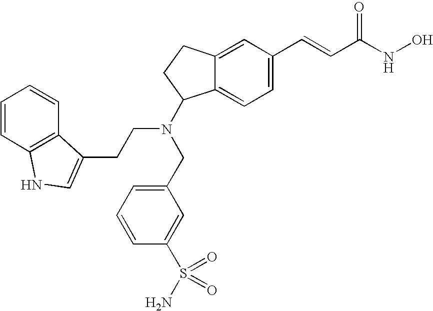 | | | |
| 407 | 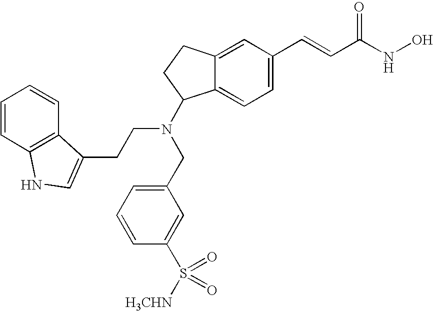 | | | |
| 408 | 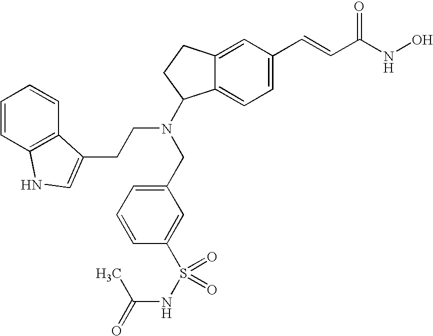 | | | |
| 409 | 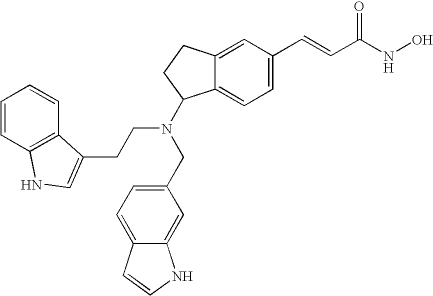 | D, E, F104, 409 | 2.32 (A) | 491.1 |
| 410 | 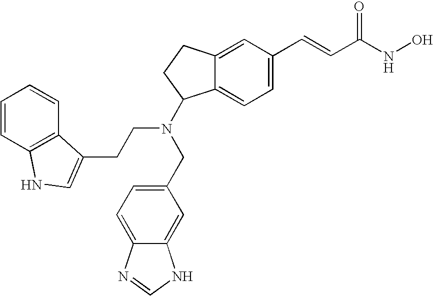 | | | |
| 411 | 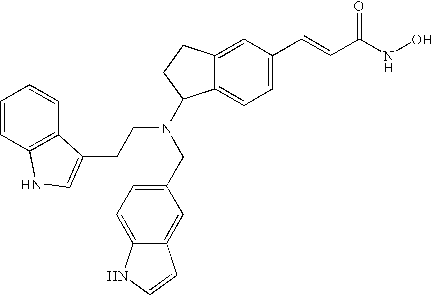 | D, E, F105, 411 | 2.65 (A) | 491.1 |
| 412 | 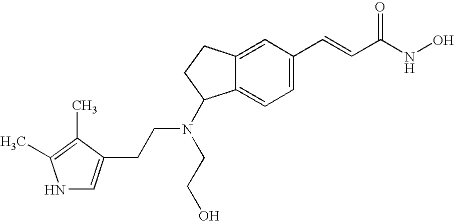 | | | |
| 413 | 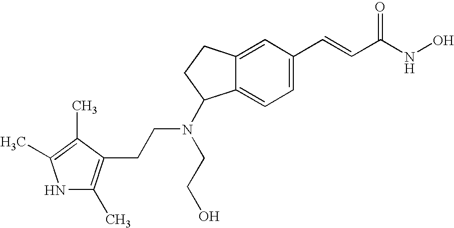 | | | |
| 414 | 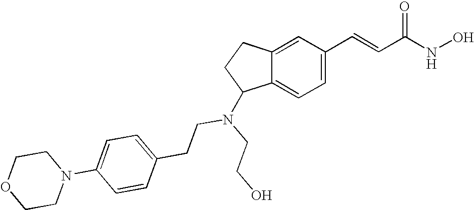 | | | |
| 415 | 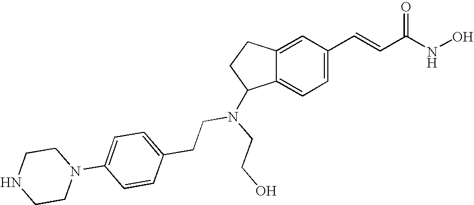 | | | |
| 416 | 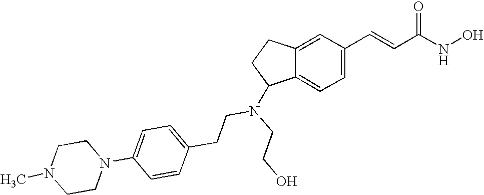 | | | |
| 417 | 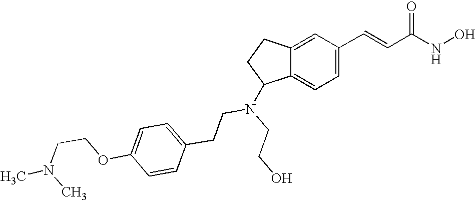 | | | |
| 418 | 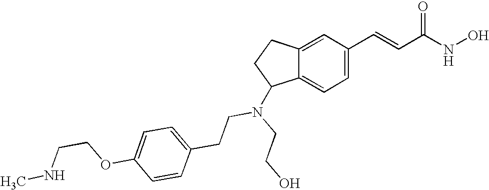 | | | |
| 419 | 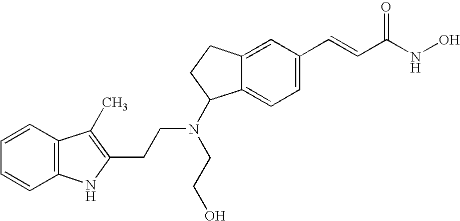 | | | |
| 420 | 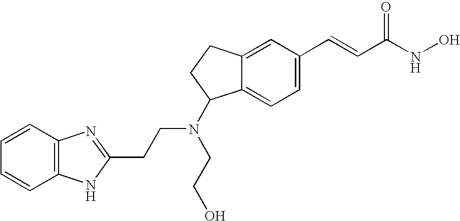 | | | |
| 421 | 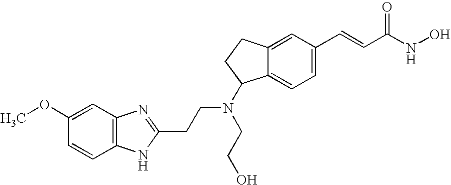 | | | |
| 422 | 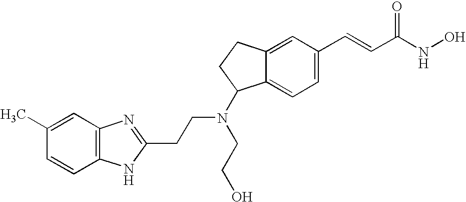 | | | |
| 423 | 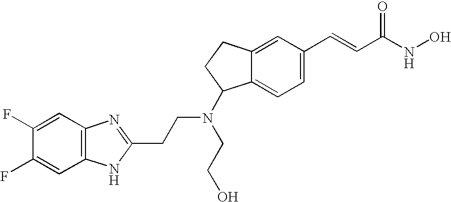 | | | |
| 424 | 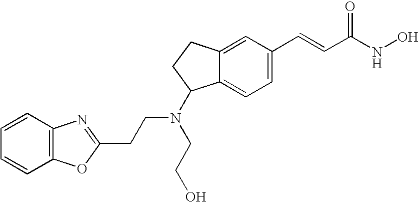 | | | |
| 425 | 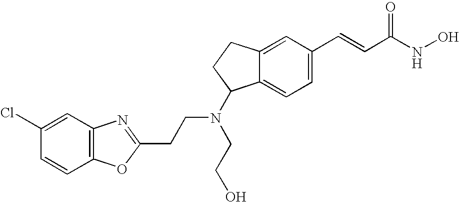 | | | |
| 426 | 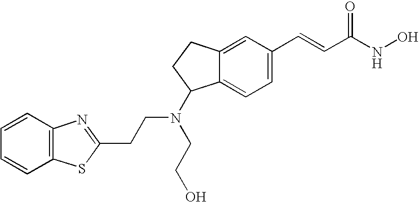 | | | |
| 427 | 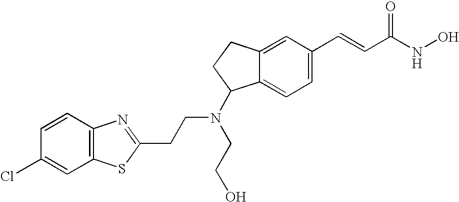 | | | |
| 428 | 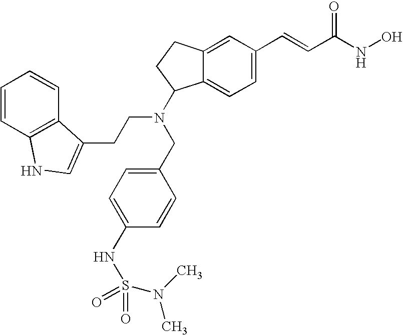 | D, E, F106 (via AH5), 428 | 1.26 (A) | 574.0 |
| 429 | 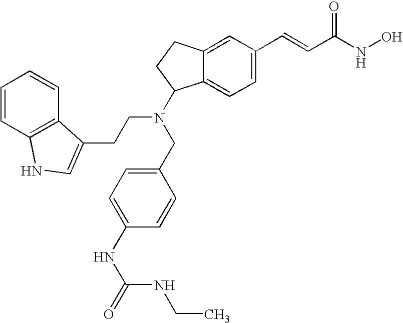 | D, E, F107 (via AH6), 429 | 2.51 (A) | 538.2 |
| 430 | 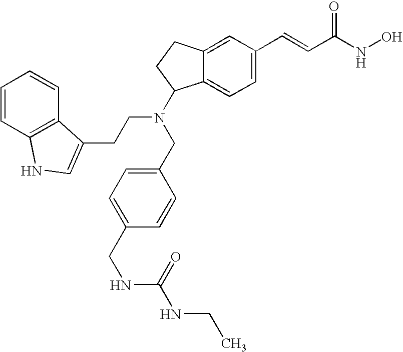 | D, E, F171 (via AH7), 430 | 2.07 (A) | 552.2 |
| 431 | 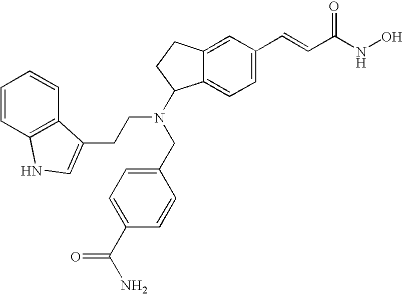 | D, E, F108 (via AH8), 431 | 1.93 (A) | 495.1 |
| 432 | 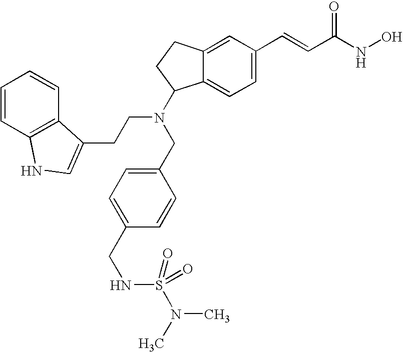 | D, E, F109 (via AH9), 432 | 2.63 (A) | 588.1 |
| 433 | 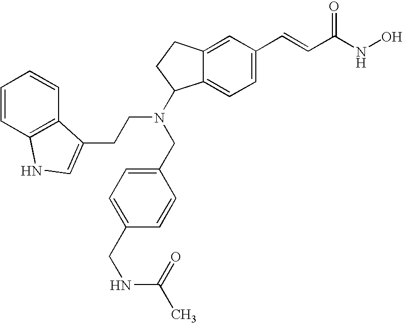 | | | |
| 434 | 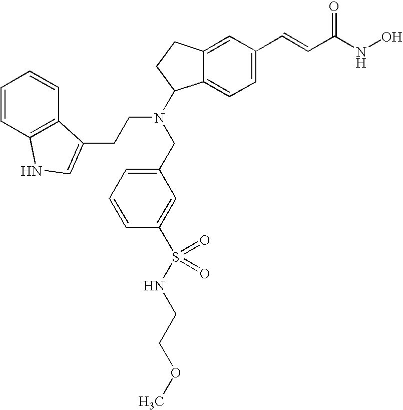 | | | |
| 435 | 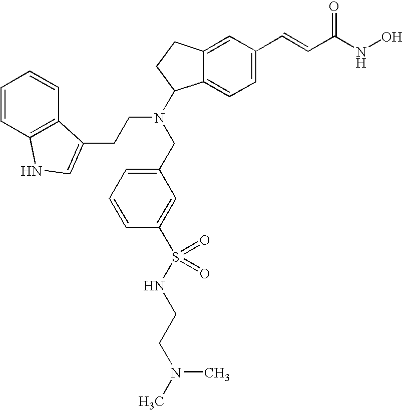 | | | |
| 436 | 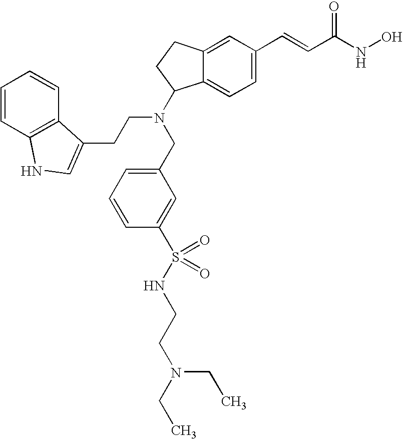 | | | |
| 437 | 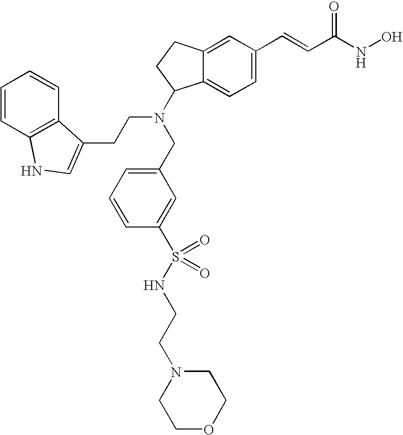 | | | |
| 438 | 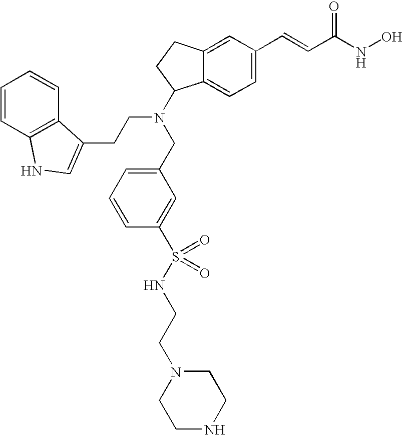 | | | |
| 439 | 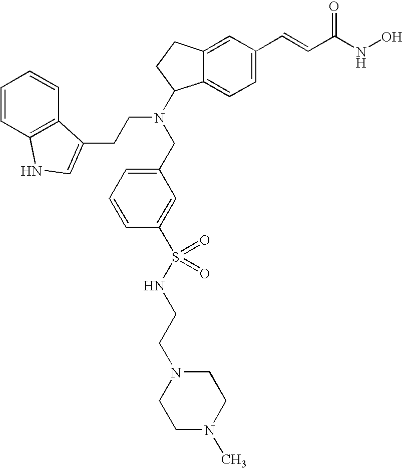 | | | |
| 440 | 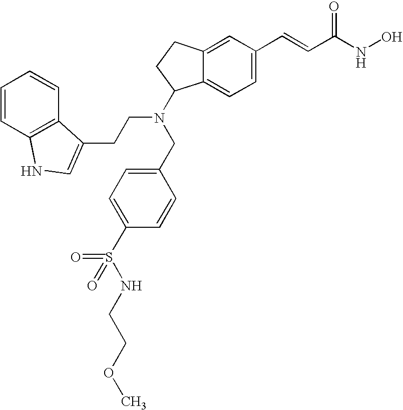 | | | |
| 441 | 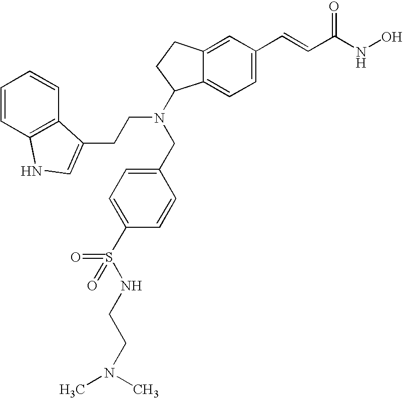 | | | |
| 442 | 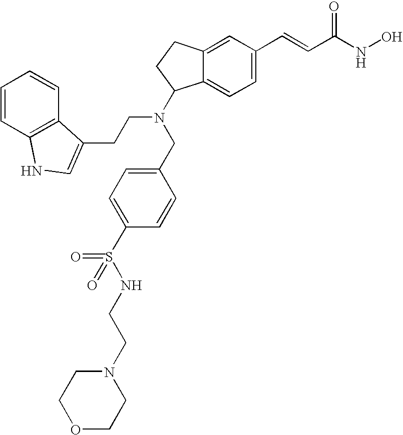 | | | |
| 443 | 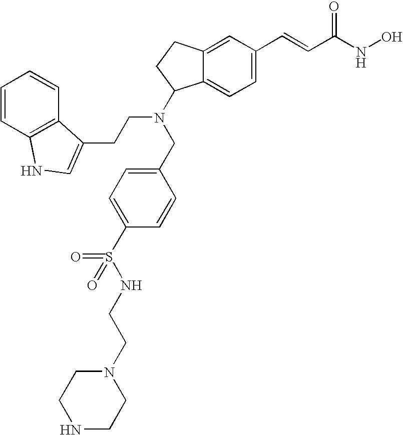 | | | |
| 444 | 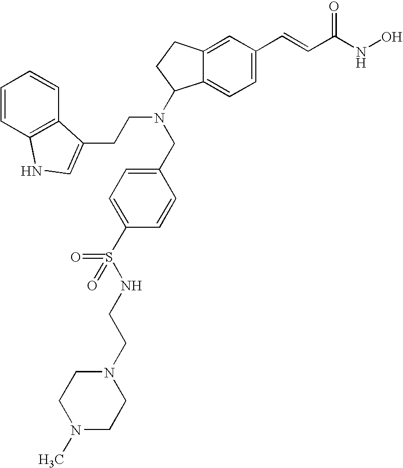 | | | |
| 445 | 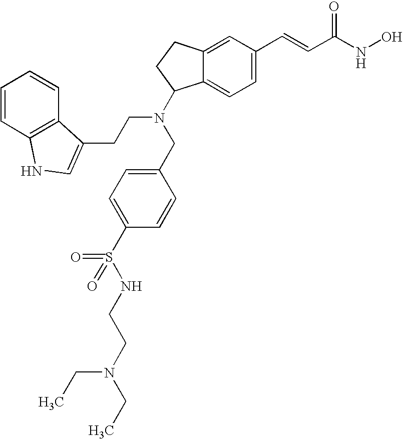 | | | |
| 446 | 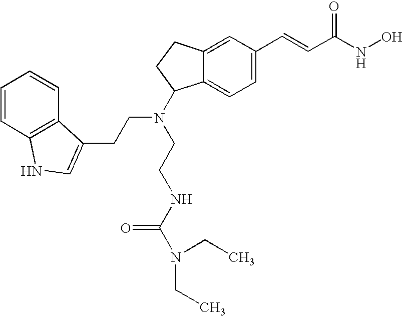 | D, E, F2, T, N5, 446 | 1.67 (A) | 504.2 |
| 447 | 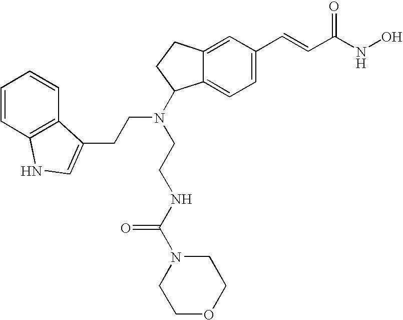 | D, E, F2, T, N6, 447 | 1.07 (A) | 518.1 |
| 448 | 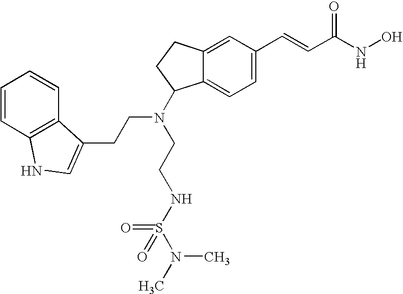 | D, E, F2, T, AA, 448 | 1.22 (A) | 512.1 |
| 449 | 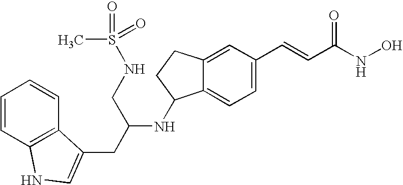 | | | |
| 450 | 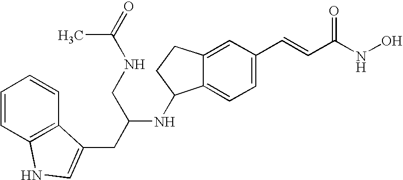 | | | |
| 451 | 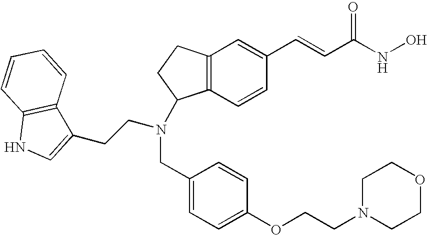 | | | |
| 452 | 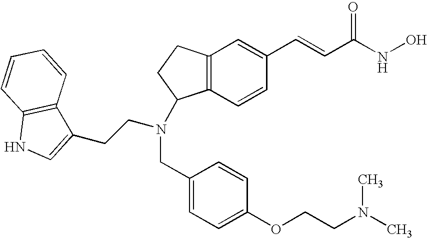 | | | |
| 453 | 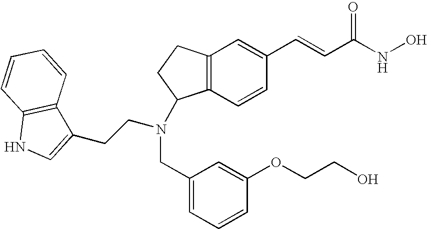 | D, E, F110, 453 | 1.14 (A) | 512.1 |
| 454 | 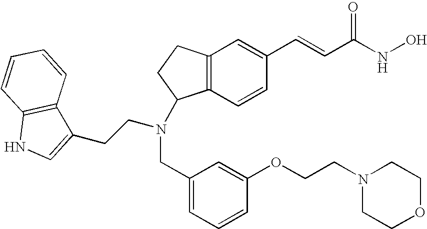 | | | |
| 455 | 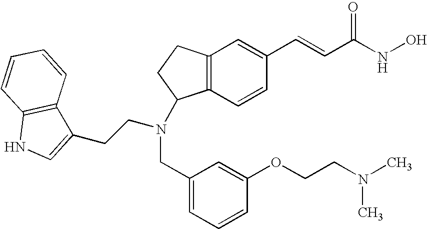 | | | |
| 456 | 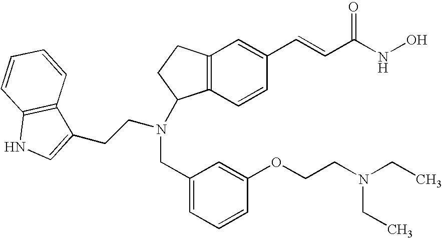 | D, E, F111, 456 | 0.94 (A) | 567.3 |
| 457 | 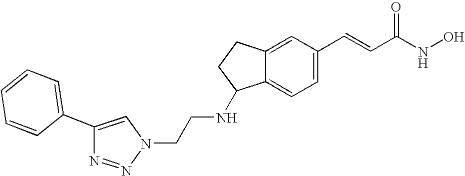 | | | |
| 458 | 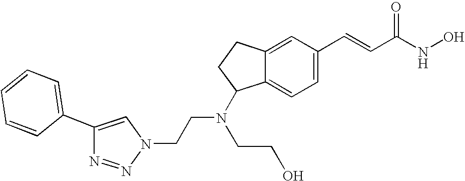 | | | |
| 459 | 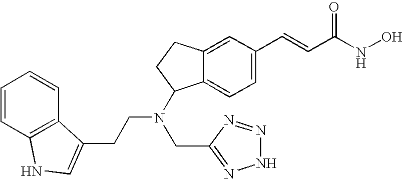 | | | |
| 460 | 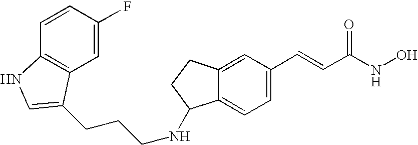 | D, Q53, 460 | 1.19 (A) | 394.0 |
| 461 | 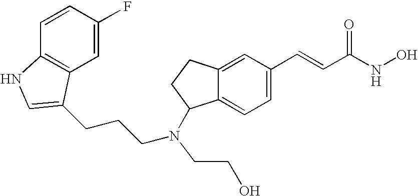 | | | |
| 462 | 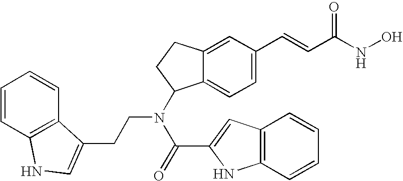 | D, E, X, 462 | 3.46 (A) | 505.0 |
| 463 | 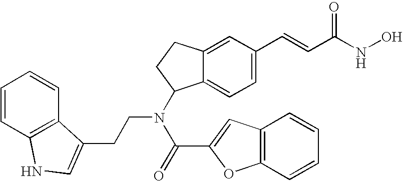 | D, E, X2, 463 | 3.49 (A) | 506.0 |
| 464 | 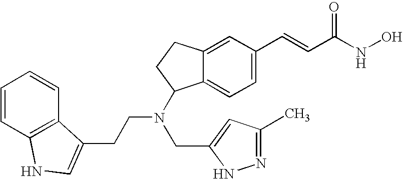 | | | |
| 465 | 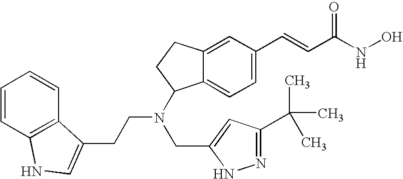 | | | |
| 466 | 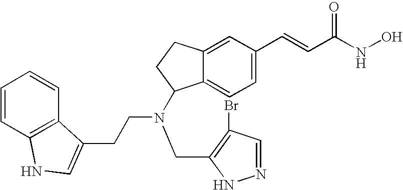 | | | |
| 467 | 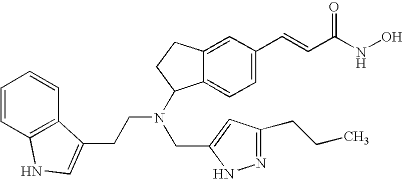 | | | |
| 468 | 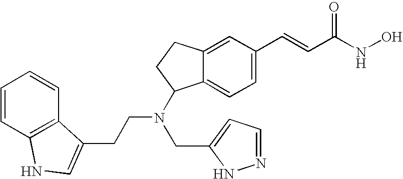 | | | |
| 469 | 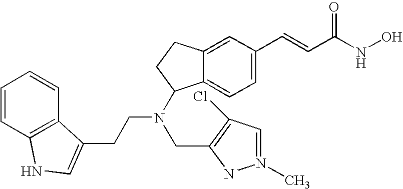 | D, E, F112, 469 | 1.27 (A) | 490.1 |
| 470 | 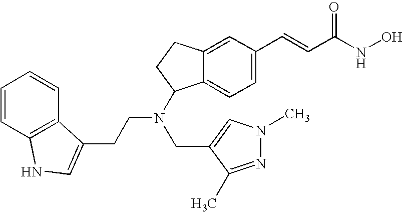 | D, E, F113, 470 | 2.26 (A) | 470.1 |
| 471 | 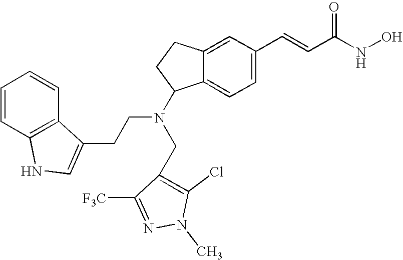 | D, E, F114, 471 | 1.05 (A) | 557.9 |
| 472 | 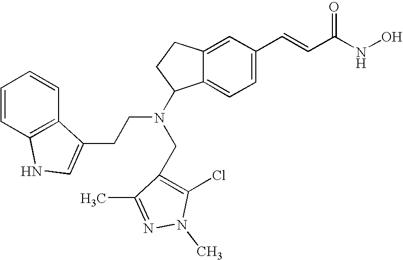 | D, E, F115, 472 | 2.43 (A) | 504.1 |
| 473 | 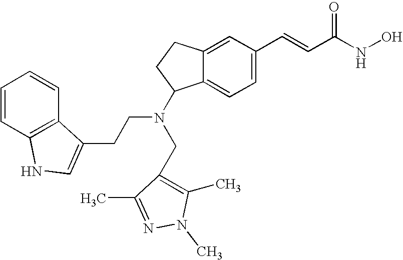 | D, E, F116, 473 | 1.02 (A) | 484.0 |
| 474 | 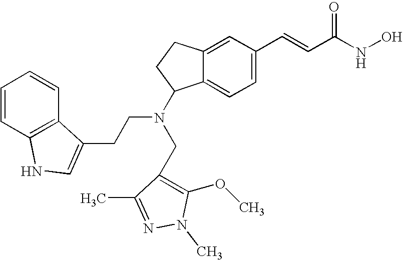 | | | |
| 475 | 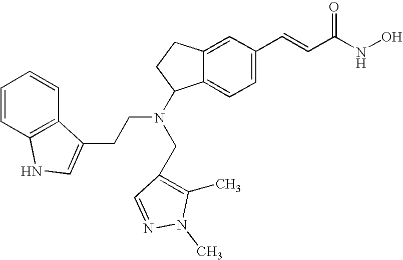 | D, E, F117, 475 | 2.25 (A) | 470.1 |
| 476 | 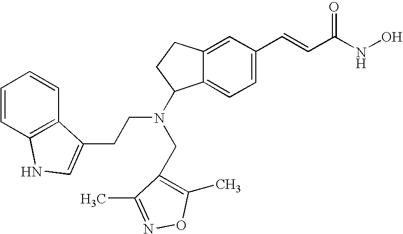 | D, E, F118, 476 | 1.32 (A) | 471.0 |
| 477 | 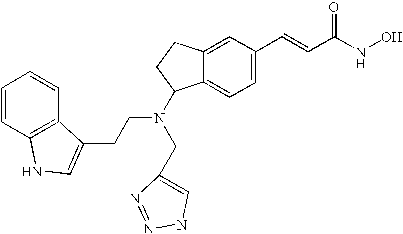 | D, E, F119, 477 | 0.99 (A) | 460.0 |
| 478 | 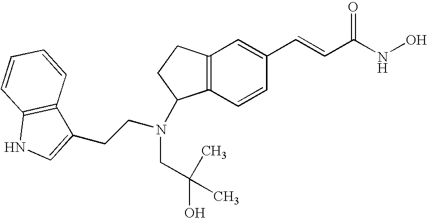 | | | |
| 479 | 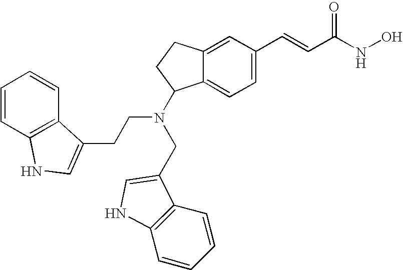 | | | |
| 480 | 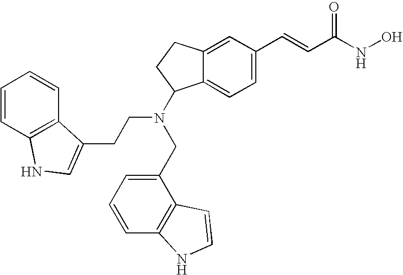 | | | |
| 481 | 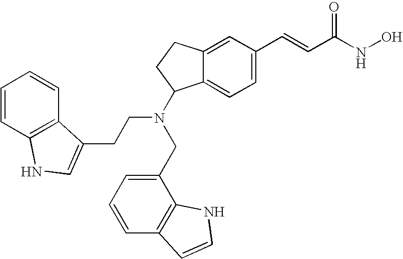 | | | |
| 482 | 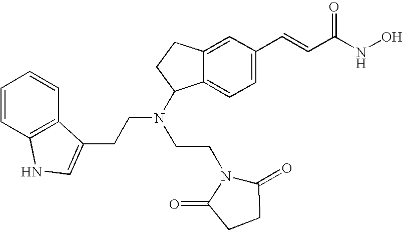 | | | |
| 483 | 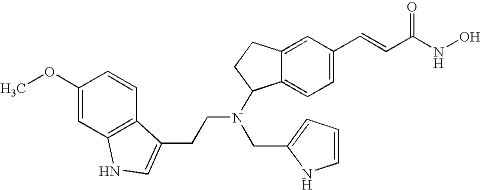 | D, Q13, F120, 483 | 2.11 (A) | 470.9 |
| 484 | 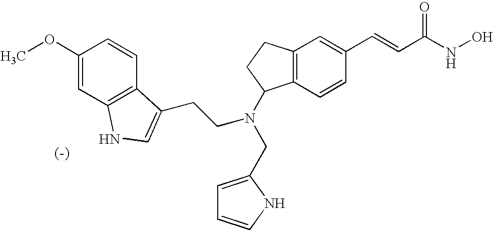 | D, Q13, F120, R43, 484 | 2.11 (A) | 470.9 |
| 485 | 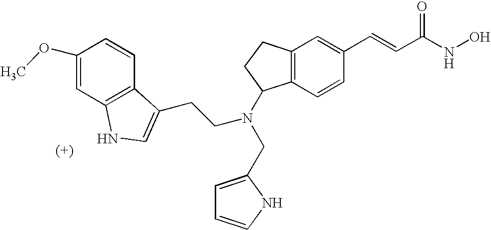 | D, Q13, F120, R44, 485 | 2.12 (A) | 470.9 |
| 486 | 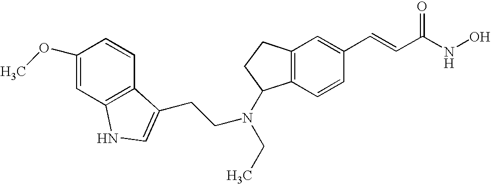 | D, Q13, F121, 486 | 1.84 (A) | 420.1 |
| 487 | 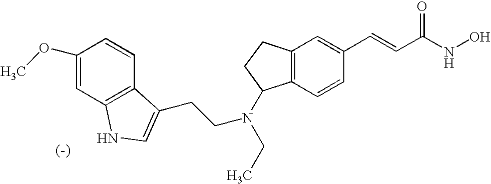 | D, Q13, F121, R21, 487 | 1.79 (A) | 420.1 |
| 488 | 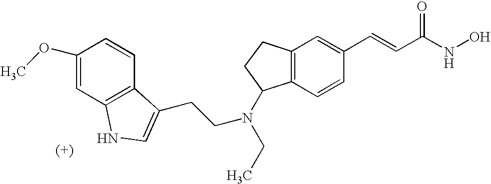 | D, Q13, F121, R22, 488 | 1.80 (A) | 420.1 |
| 489 | 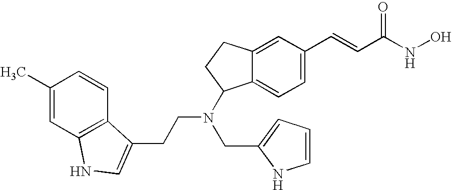 | D, Q54, F122, 489 | 2.27 (A) | 454.9 |
| 490 | 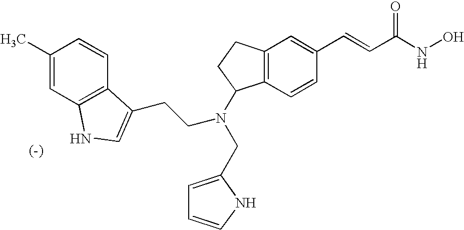 | D, Q54, F122, R37, 490 | 2.25 (A) | 454.9 |
| 491 | 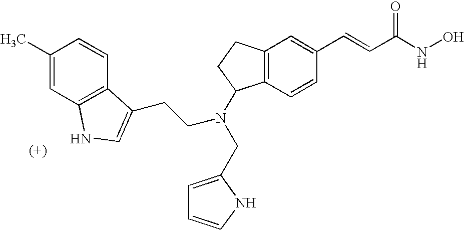 | D, Q54, F122, R38, 491 | 2.26 (A) | 454.9 |
| 492 | 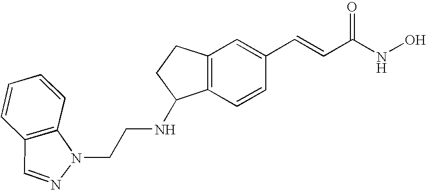 | D, Q55, 492 | 1.80 (A) | 363.0 |
| 493 | 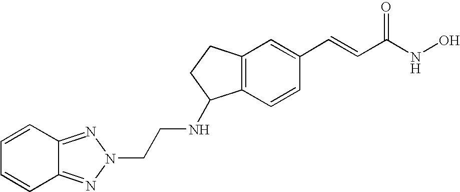 | D, Q56, 493 | 1.72 (A) | 363.0 |
| 494 | 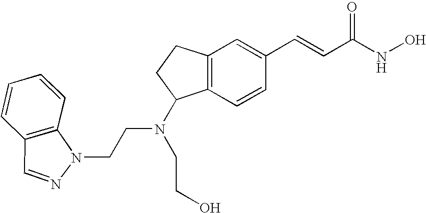 | | | |
| 495 | 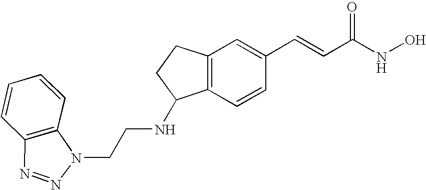 | | | |
| 496 | 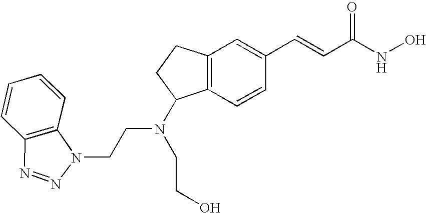 | | | |
| 497 | 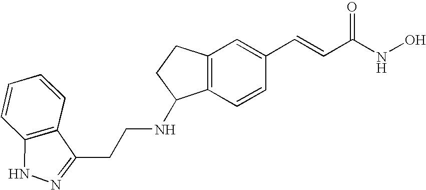 | | | |
| 498 | 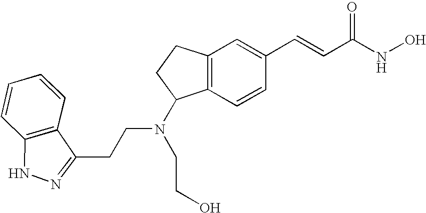 | | | |
| 499 | 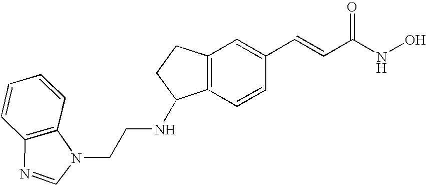 | D, Q57, 499 | 1.07 (A) | 363.1 |
| 500 | 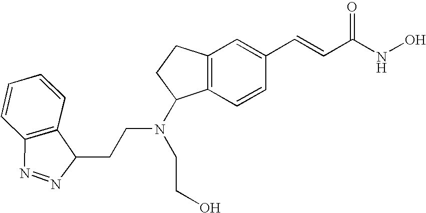 | | | |
| 501 | 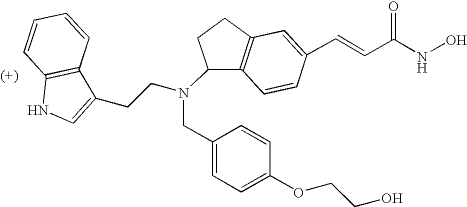 | D, E, F69, R19, 501 | 0.95 (A) | 512.1 |
| 502 | 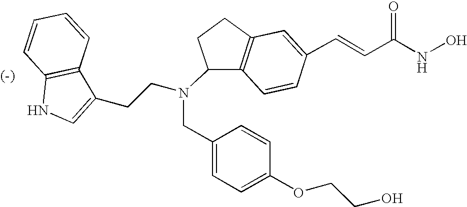 | D, E, F69, R20, 502 | 1.01 (A) | 512.1 |
| 503 | 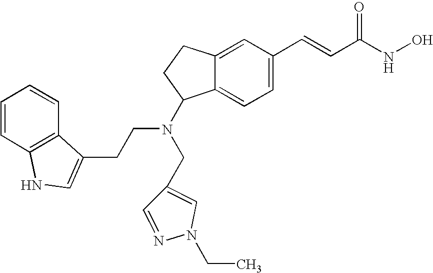 | D, E, F123, 503 | 1.97 (A) | 470.1 |
| 504 | 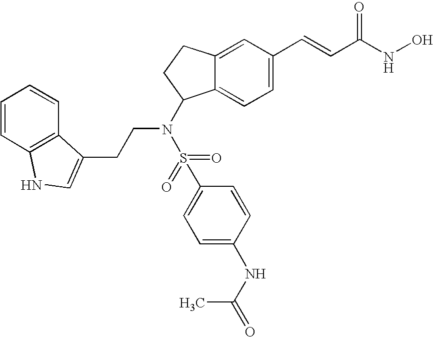 | D, E, O4, 504 | 2.26 (A) | 558.9 |
| 505 | 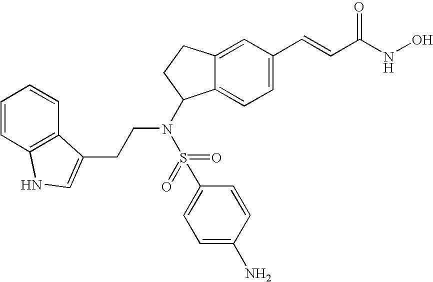 | D, E, O4, AB, 505 | 3.62 (A) | 517.1 |
| 506 | 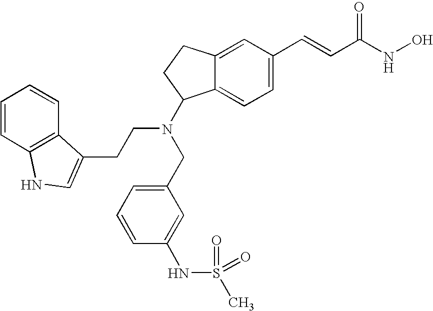 | D, E, F124 (via AH10), 506 | 2.17 (A) | 545.1 |
| 507 | 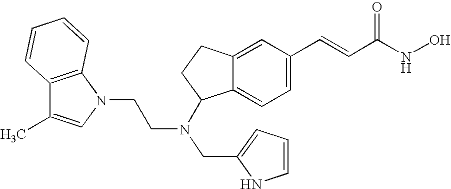 | D, Q51, F125, 507 | 2.25 (A) | 455.0, |
| 508 | 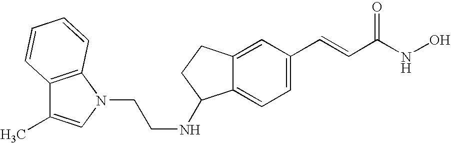 | D, Q51, 508 | 1.08 (A) | 375.9 |
| 509 | 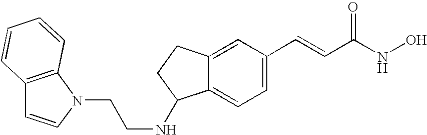 | D, Q50, 509 | 1.46 (A) | 362.0 |
| 510 | 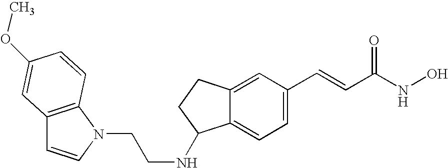 | D, Q52, 510 | 0.93 (A) | 392.0 |
| 511 | 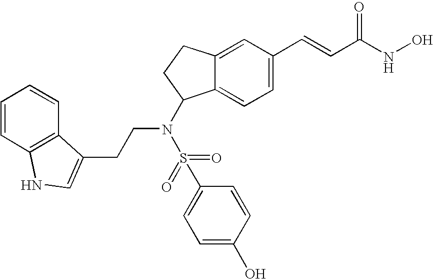 | D, E, O3, Y, 511 | 2.73 (A) | 518.0 |
| 512 | 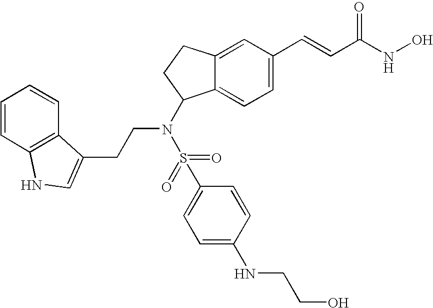 | D, E, O4, AB, AC, 512 | 3.08 (A) | 561.1 |
| 513 | 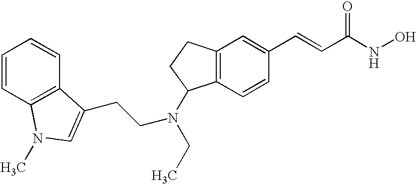 | D, Q58, F169, 513 | 2.09 (A) | 404.1 |
| 514 | 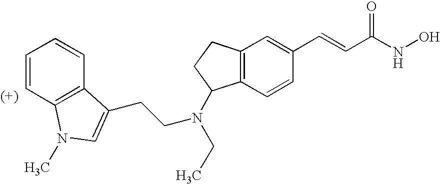 | D, Q58, F169, R49, 514 | 2.02 (A) | 404.1 |
| 515 | 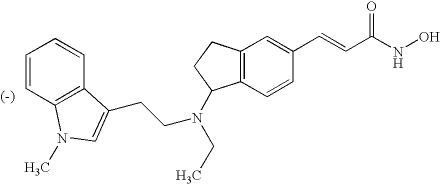 | D, Q58, F169, R50, 515 | 2.05 (A) | 404.1 |
| 516 | 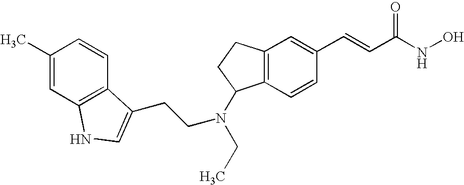 | D, Q54, F127, 516 | 2.01 (A) | 404.1 |
| 517 | 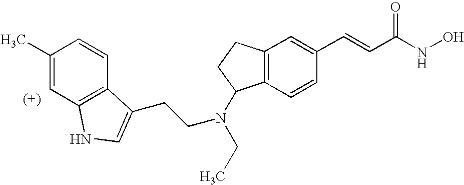 | D, Q54, F127, R51, 517 | 1.85 (A) | 404.1 |
| 518 | 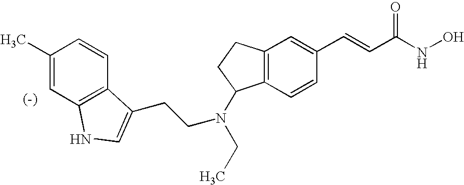 | D, Q54, F127, R52, 518 | 1.80 (A) | 404.1 |
| 519 | 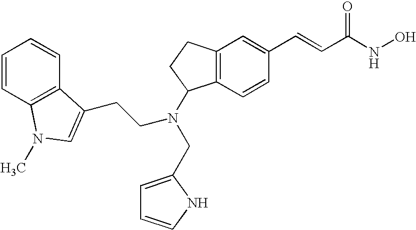 | D, Q58, F128, 519 | 2.29 (A) | 454.9 |
| 520 | 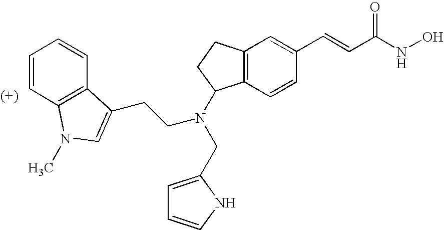 | D, Q58, F128, R53, 520 | | |
| 521 | 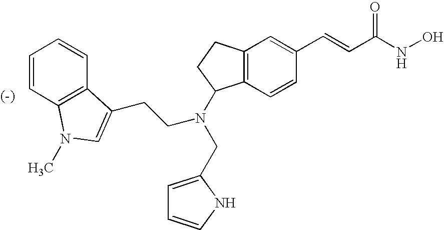 | D, Q58, F128, R54, 521 | | |
| 522 | 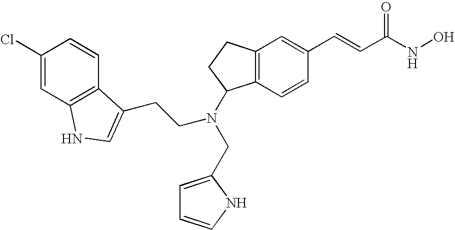 | D, Q59, F129, 522 | 2.29 (A) | 474.9 |
| 523 | 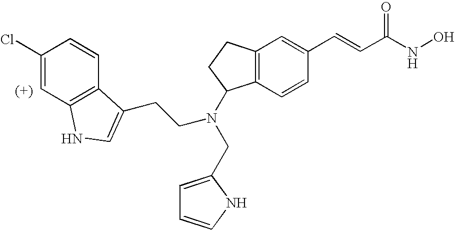 | D, Q59, F129, R55, 523 | 2.28 (A) | 474.9 |
| 524 | 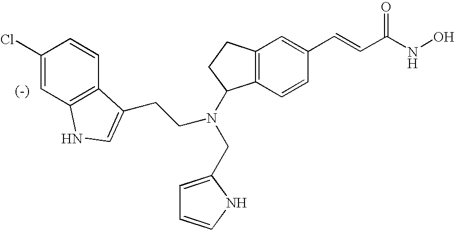 | D, Q59, F129, R56, 524 | 2.30 (A) | 474.9 |
| 525 | 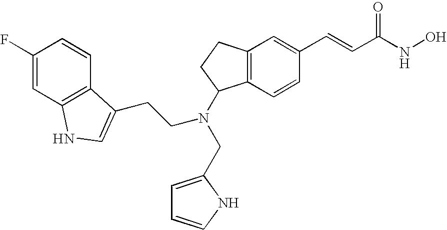 | D, Q60, F130, 525 | 2.20 (A) | 458.9 |
| 526 | 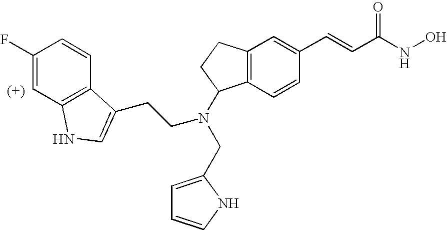 | D, Q60, F130, R57, 526 | 2.17 (A) | 458.9 |
| 527 | 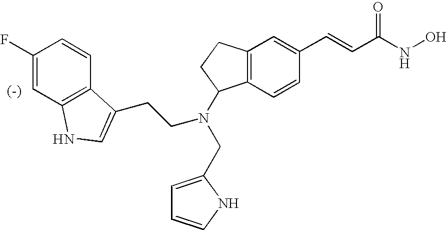 | D, Q60, F130, R58, 527 | 2.19 (A) | 458.9 |
| 528 | 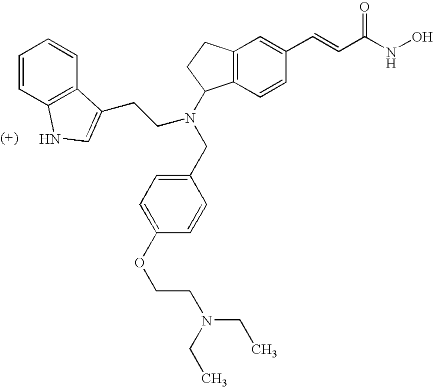 | D, E, F76, R59, 528 | 1.66 (A) | 567.3 |
| 529 | 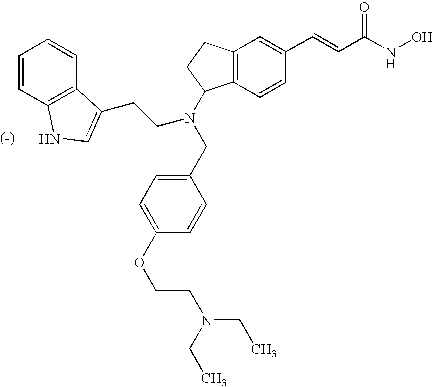 | D, E, F76, R60, 529 | 1.65 (A) | 567.3 |
| 530 | 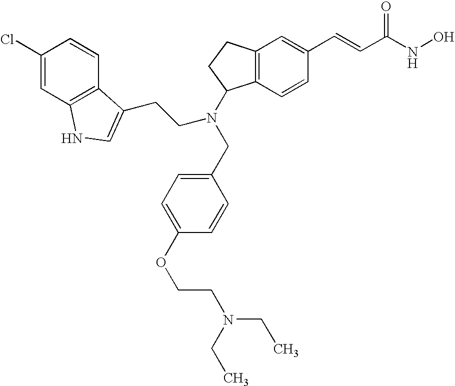 | D, Q59, F131, 530 | 1.94 (A) | 601.4 |
| 531 | 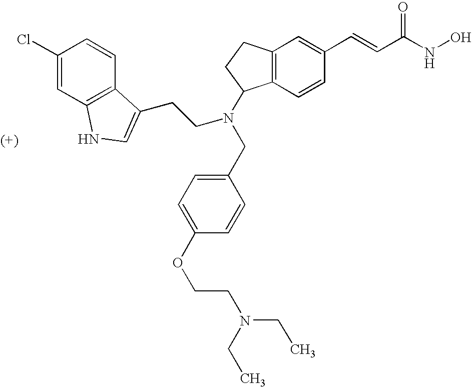 | D, Q59, F131, R61, 531 | 1.93 (A) | 601.4 |
| 532 | 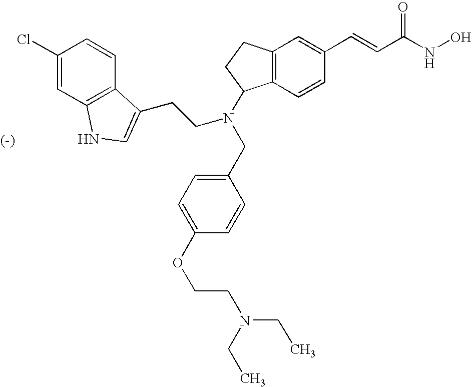 | D, Q59, F131, R62, 532 | 1.94 (A) | 601.4 |
| 533 | 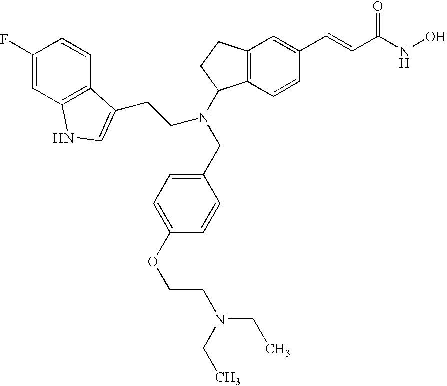 | D, Q60, F132, 533 | 1.85 (A) | 585.3 |
| 534 | 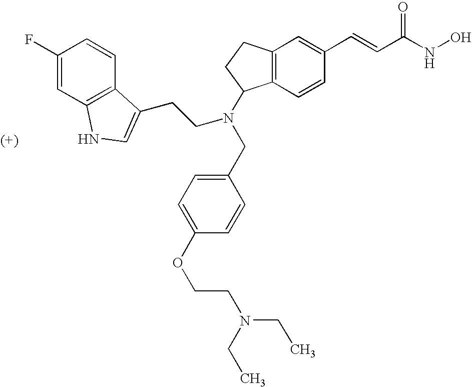 | D, Q60, F132, R63, 534 | 1.90 (A) | 585.2 |
| 535 | 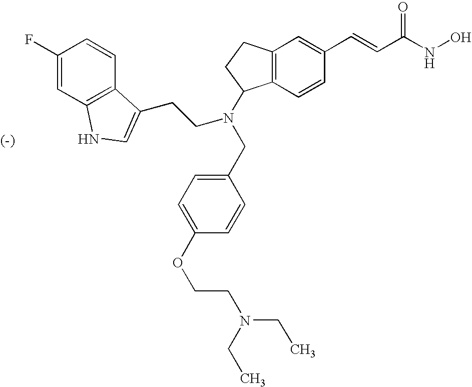 | D, Q60, F133, R64, 535 | 1.89 (A) | 585.2 |
| 536 | 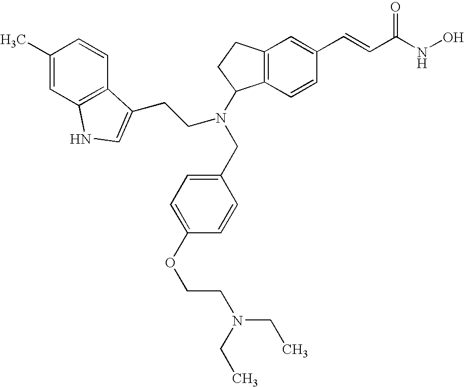 | D, Q54, F133, 536 | 1.89 (A) | 581.3 |
| 537 | 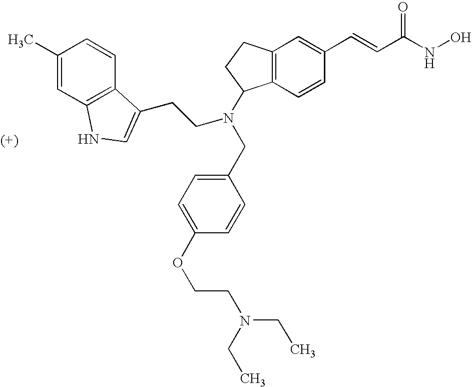 | D, Q54, F133, R65, 537 | 1.78 (A) | 581.3 |
| 538 | 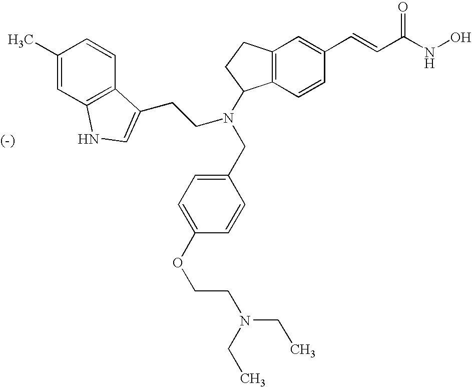 | D, Q54, F133, R66, 538 | 1.94 (A) | 581.2 |
| 539 | 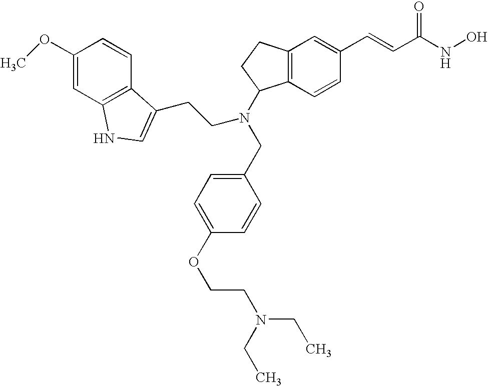 | D, Q13, F134, 539 | 1.86 (A) | 597.2 |
| 540 | 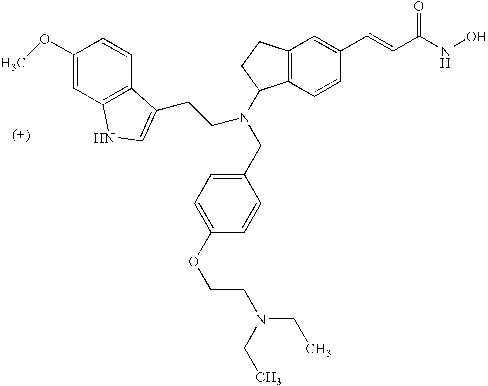 | D, Q13, F134, R67, 540 | 1.84 (A) | 597.2 |
| 541 | 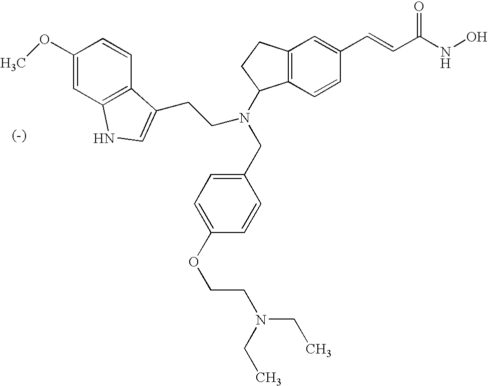 | D, Q13, F134, R68, 541 | 1.84 (A) | 597.2 |
| 542 | 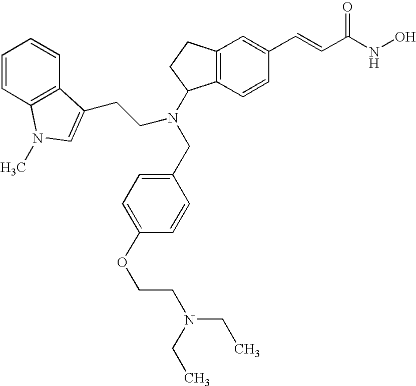 | D, Q58, F135, 542 | 1.95 (A) | 581.3 |
| 543 | 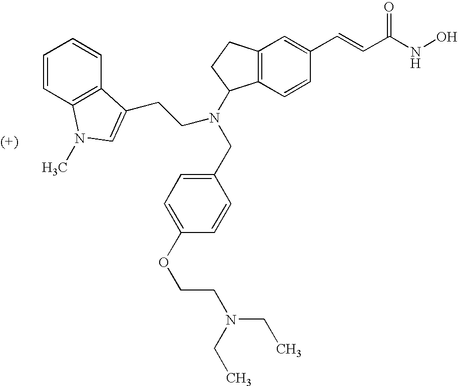 | D, Q58, F135, R69, 543 | 1.98 (A) | 581.2 |
| 544 | 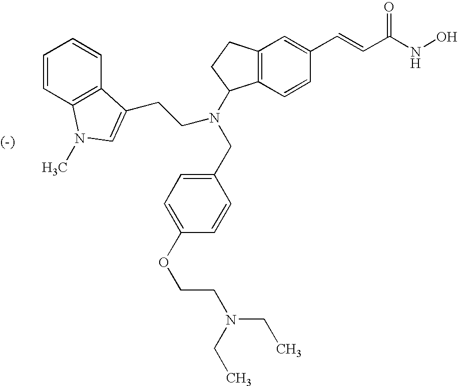 | D, Q58, F135, R70, 544 | 1.99 (A) | 581.2 |
| 545 | 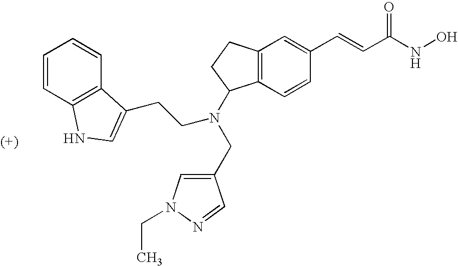 | D, E, F123, R71, 545 | 1.80 (A) | 470.1 |
| 546 | 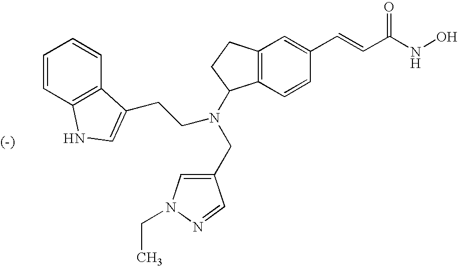 | D, E, F123, R72, 546 | 1.77 (A) | 470.1 |
| 547 | 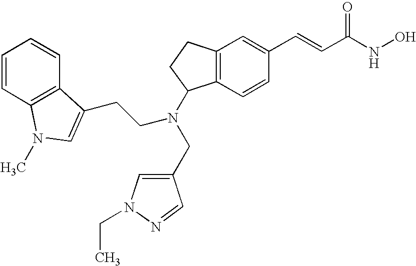 | D, Q58, F136, 547 | 2.07 (A) | 484.1 |
| 548 | 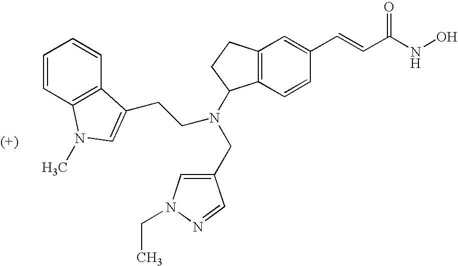 | D, Q58, F136, R73, 548 | 2.12 (A) | 484.2 |
| 549 | 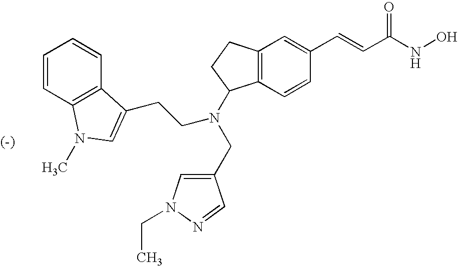 | D, Q58, F136, R74, 549 | 2.12 (A) | 484.2 |
| 550 | 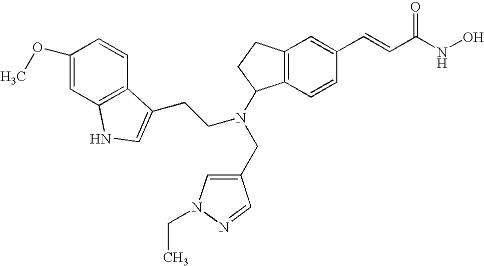 | D, Q13, F137, 550 | 1.97 (A) | 500.0 |
| 551 | 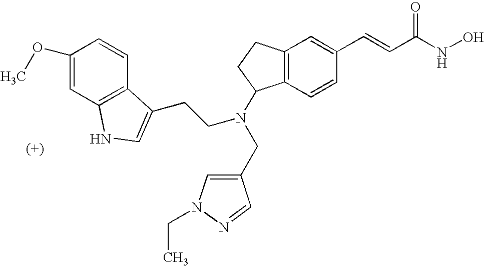 | D, Q13, F137, R75, 551 | 1.99 (A) | 500.0 |
| 552 | 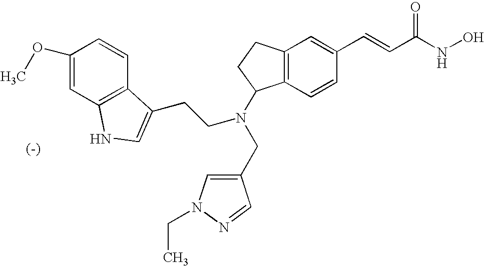 | D, Q13, F137, R76, 552 | 1.99 (A) | 500.0 |
| 553 | 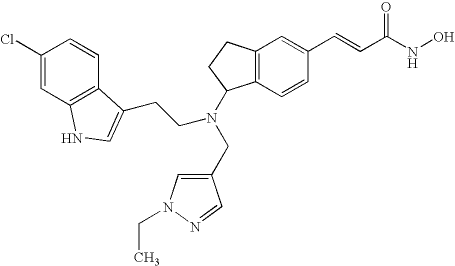 | D, Q59, F138, 553 | 2.16 (A) | 504.0 |
| 554 | 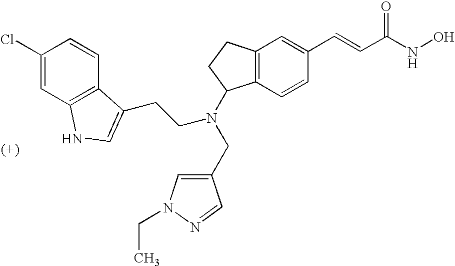 | D, Q59, F138, R77, 554 | 2.17 (A) | 504.2 |
| 555 | 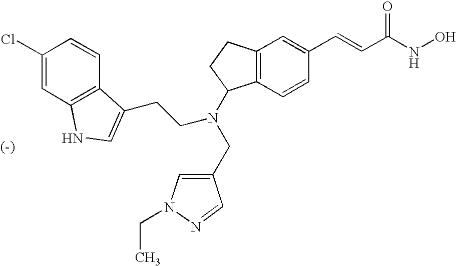 | D, Q59, F138, R78, 555 | 2.17 (A) | 504.2 |

| | | | HPLC RT | |
|---|---|---|---|---|
| Compound | | Synthetic | (min) | |
| Example | Structure | sequence | (method) | M + H |
| 556 | 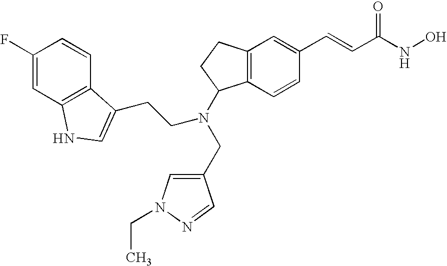 | D, Q60, F139, 556 | 2.04 (A) | 488.1 |
| 557 | 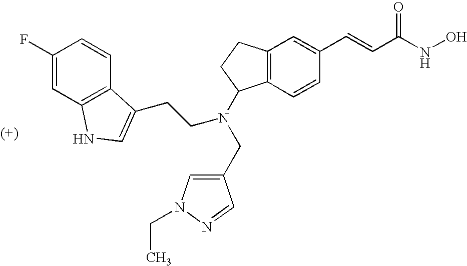 | D, Q60, F139, R79, 557 | 2.00 (A) | 488.1 |
| 558 | 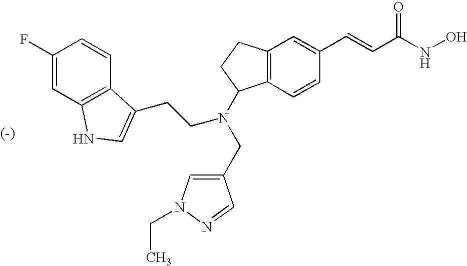 | D, Q60, F139, R80, 558 | 1.88 (A) | 488.1 |
| 559 | 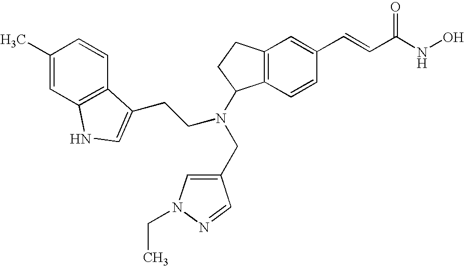 | D, Q54, F140, 559 | 2.11 (A) | 484.1 |
| 560 | 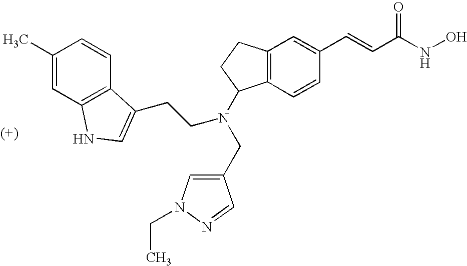 | D, Q54, F140 R81, 560 | 1.92 (A) | 484.1 |
| 561 | 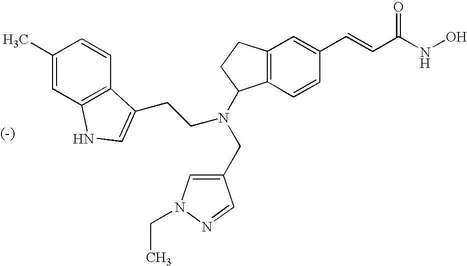 | D, Q54, F140, R82, 561 | 1.96 (A) | 484.1 |
| 562 | 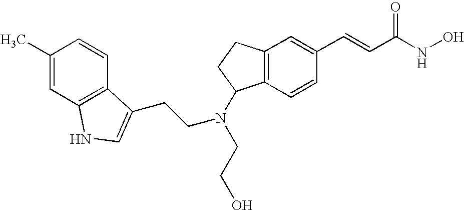 | D, Q54, F141, J57, 562 | 1.88 (A) | 420.1 |
| 563 | 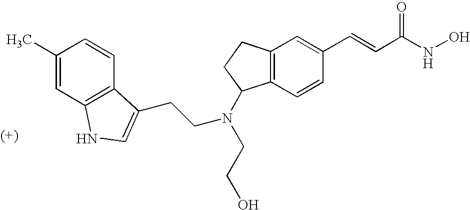 | D, Q54, F141, J57, R83, 563 | 1.87 (A) | 420.1 |
| 564 | 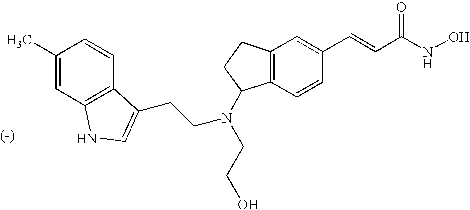 | D, Q54, F141, J57, R84, 564 | 1.88 (A) | 420.1 |
| 565 | 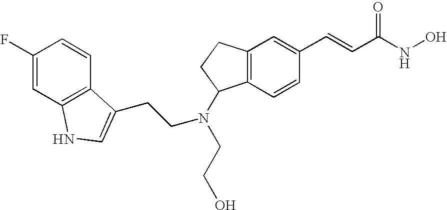 | D, Q60, F142, J58, 565 | 1.75 (A) | 424.0 |
| 566 | 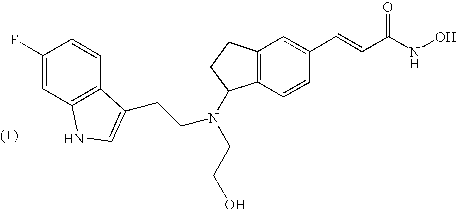 | D, Q60, F142, J58, R85, 566 | 1.72 (A) | 424.0 |
| 567 | 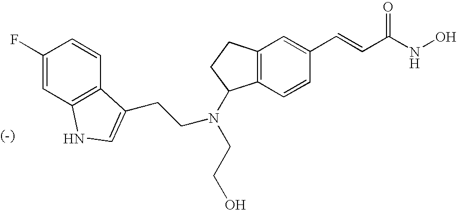 | D, Q60, F142, J58, R86, 567 | 1.69 (A) | 424.0 |
| 568 | 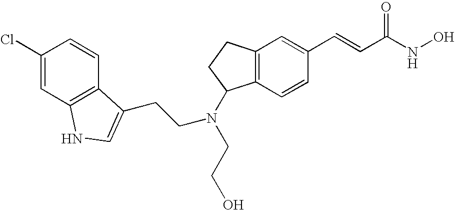 | D, Q59, F143, J59, 568 | 1.90 (A) | 440.1 |
| 569 | 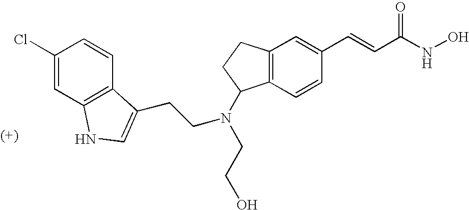 | D, Q59, F143, J59, R87, 569 | 1.95 (A) | 439.9 |
| 570 | 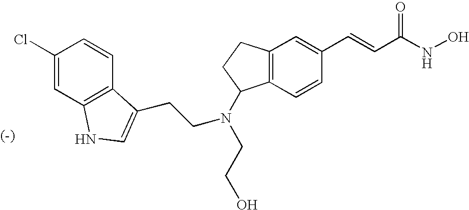 | D, Q59, F143, J59, R88, 570 | 1.95 (A) | 439.9 |
| 571 | 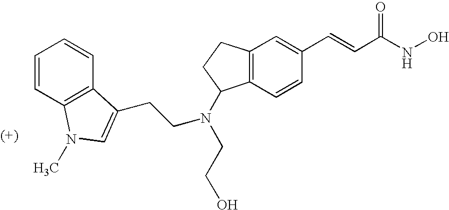 | D, E, G, G5, J5, R89, 571 | 1.84 (A) | 420.1 |
| 572 | 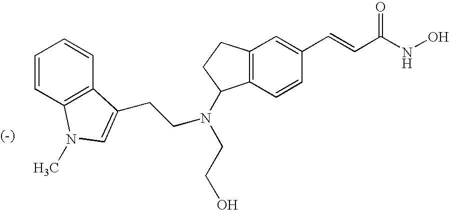 | D, E, G, G5, J5, R90, 572 | 1.87 (A) | 420.1 |
| 573 | 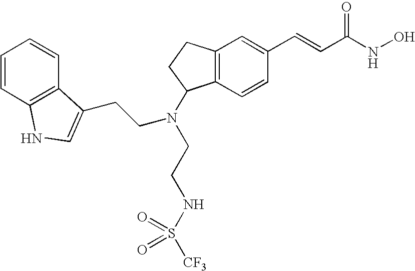 | D, E, F2, T, O5, 573 | 1.64 (A) | 537.0 |
| 574 | 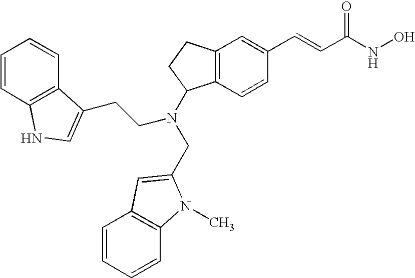 | D, E, F144, 574 | 2.50 (A) | 505.1 |
| 575 | 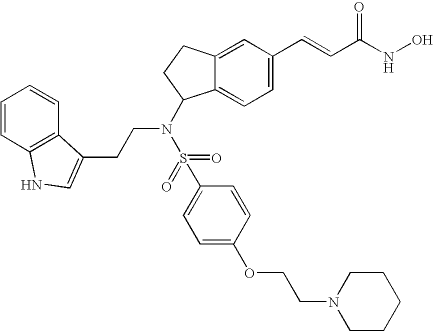 | D, E, O3, Y, Z2, 575 | 2.44 (A) | 629.4 |
| 576 | 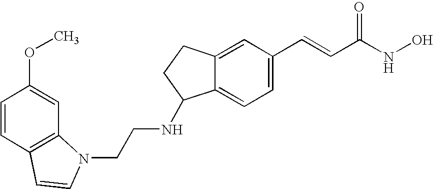 | D, Q61, 576 | 2.08 (A) | 392.0 |
| 577 | 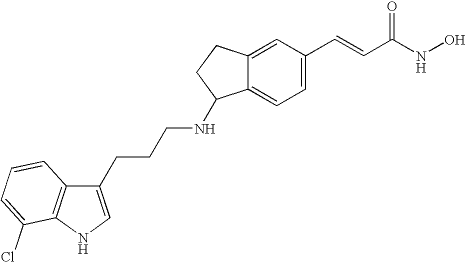 | D, Q62, 577 | 2.59 (A) | 410.0 |
| 578 | 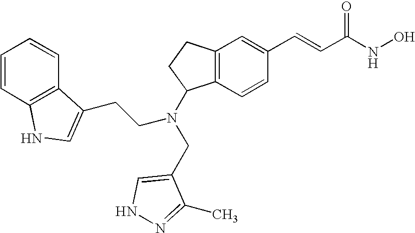 | D, E, F145, 578 | 1.93 (A) | 456.1 |
| 579 | 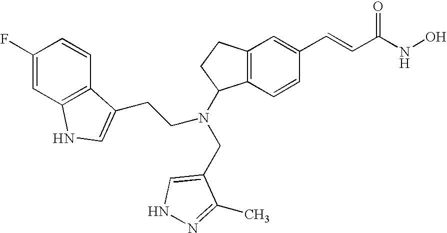 | D, E, F146, 579 | 1.76 (A) | 474.1 |
| 580 | 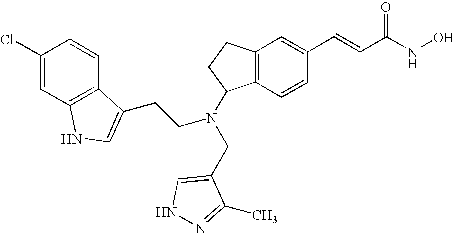 | D, E, F147, 580 | 2.10 (A) | 490.0 |
| 581 | 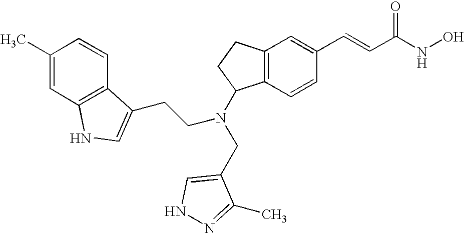 | D, E, F148, 581 | 2.42 (A) | 470.1 |
| 582 | 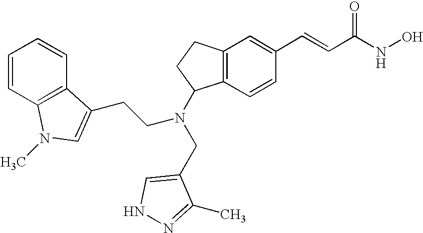 | D, E, F149, 582 | 2.45 (A) | 470.1 |
| 583 | 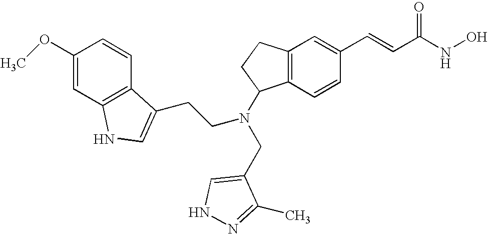 | D, E, F150, 583 | 1.69 (A) | 486.1 |
| 584 | 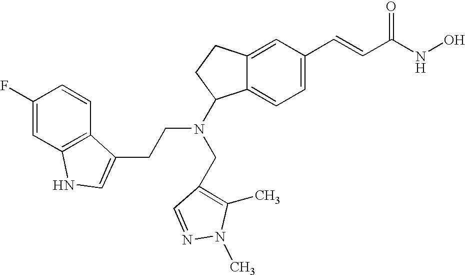 | D, E, F151, 584 | 2.02 (A) | 488.0 |
| 585 | 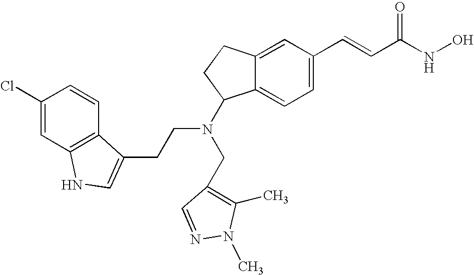 | D, E, F152, 585 | 2.54 (A) | 504.1 |
| 586 | 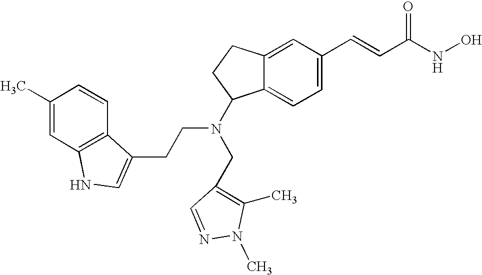 | D, E, F153, 586 | 2.47 (A) | 484.1 |
| 587 | 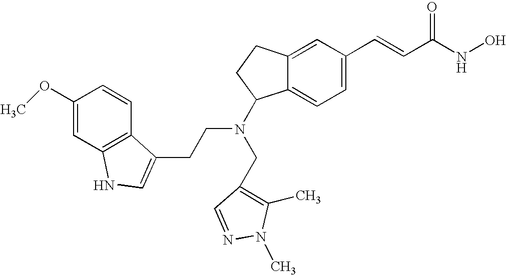 | D, E, F154, 587 | 1.81 (A) | 500.2 |
| 588 | 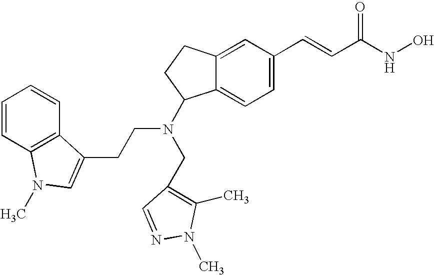 | D, E, F155, 588 | 2.14 (A) | 484.0 |
| 589 | 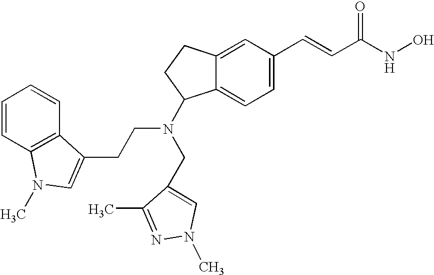 | D, E, F156, 589 | 2.13 (A) | 484.0 |
| 590 | 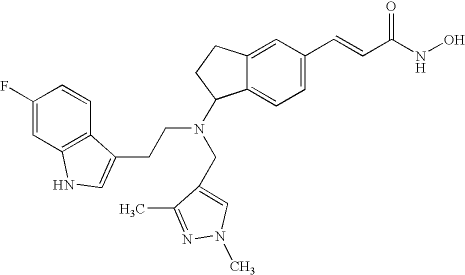 | D, E, F157, 590 | 1.82 (A) | 488.1 |
| 591 | 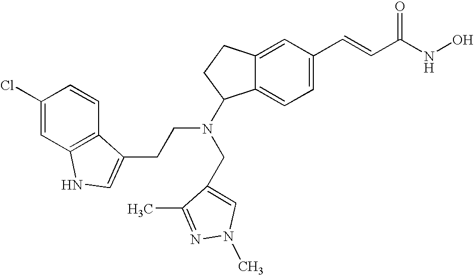 | D, E, F158, 591 | 2.53 (A) | 504.1 |
| 592 | 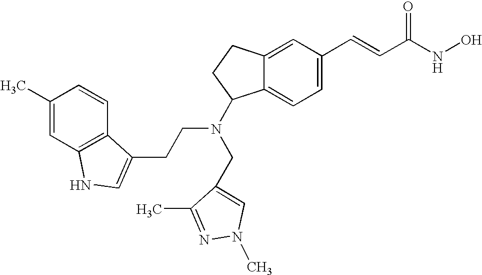 | D, E, F159, 592 | 2.46 (A) | 484.2 |
| 593 | 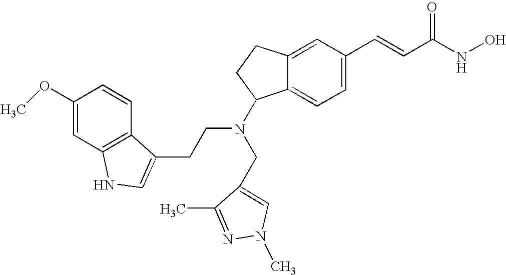 | D, E, F160, 593 | 1.80 (A) | 500.1 |
| 594 | 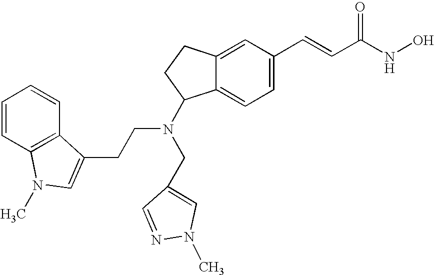 | D, E, F161, 594 | 2.09 (A) | 470.0 |
| 595 | 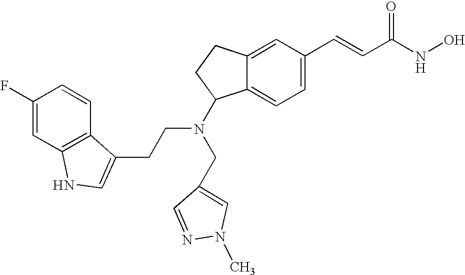 | D, E, F162, 595 | 1.97 (A) | 474.0 |
| 596 | 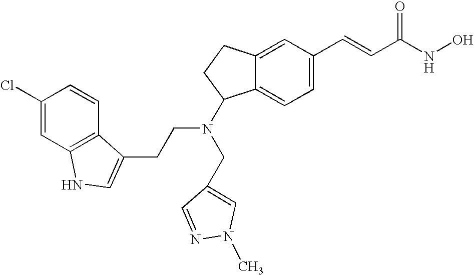 | D, E, F163, 596 | 2.07 (A) | 491.0 |
| 597 | 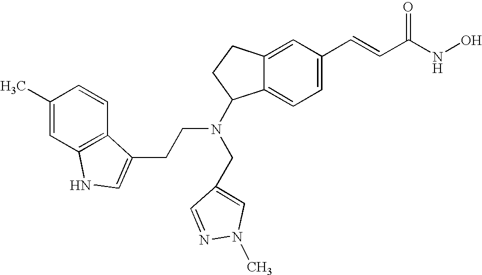 | D, E, F164, 597 | 2.03 (A) | 470.0 |
| 598 | 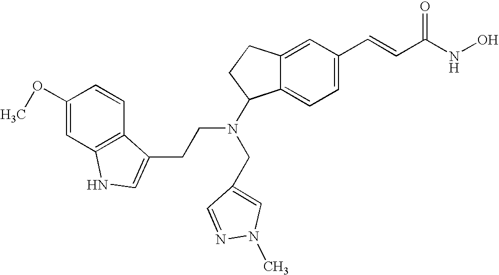 | D, E, F165, 598 | 1.90 (A) | 486.0 |
| 599 | 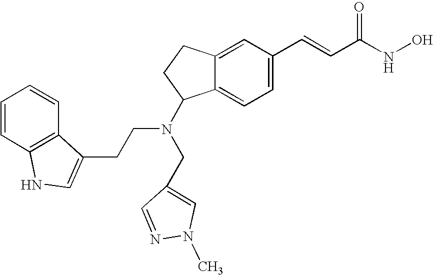 | D, E, F166, 599 | 1.91 (A) | 456.0 |
| 600 | 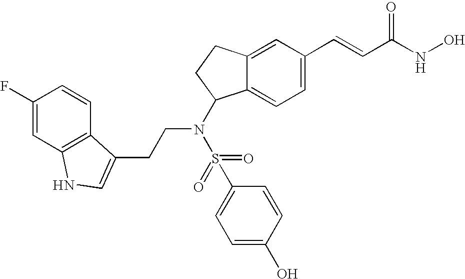 | | | |
| 601 | 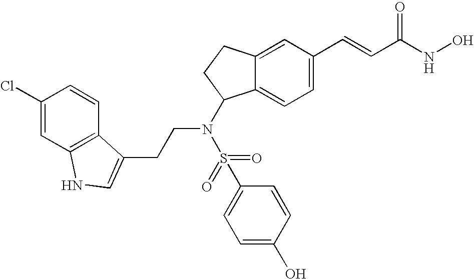 | | | |
| 603 | 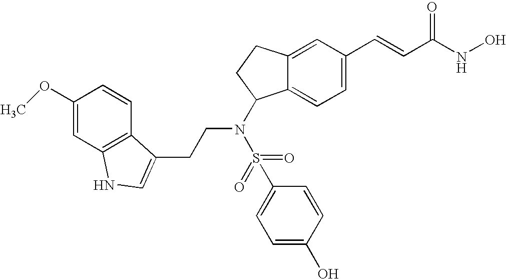 | | | |
| 604 | 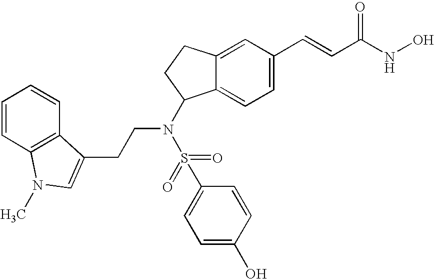 | | | |
| 605 | 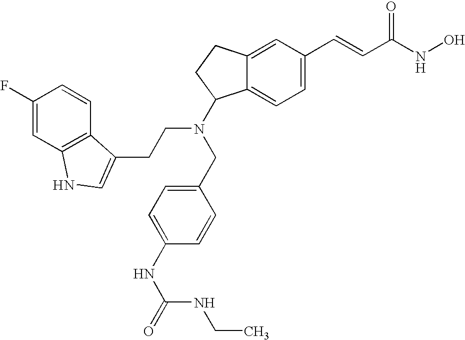 | | | |
| 606 | 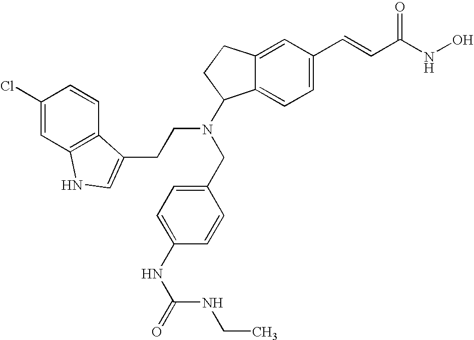 | | | |
| 607 | 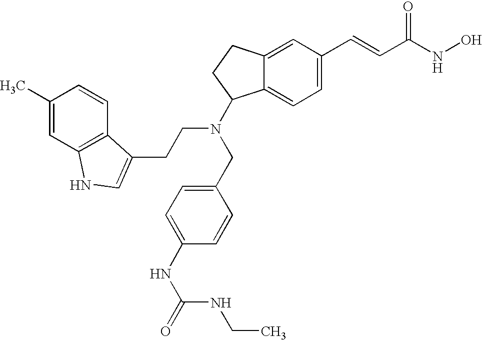 | | | |
| 608 | 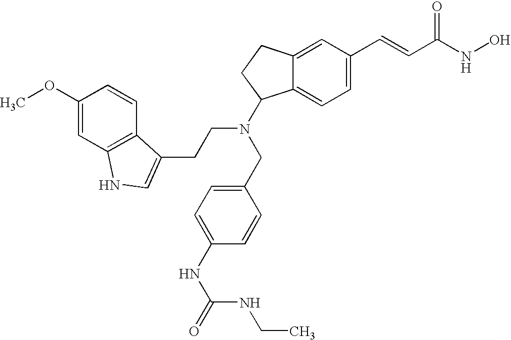 | | | |
| 609 | 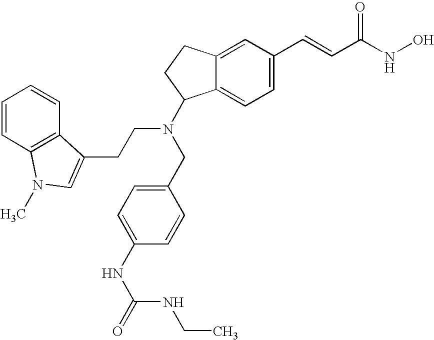 | | | |
| 610 | 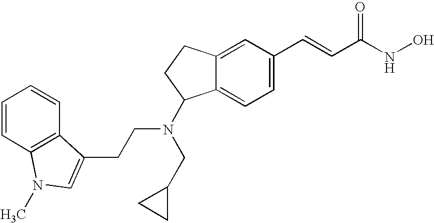 | | | |
| 611 | 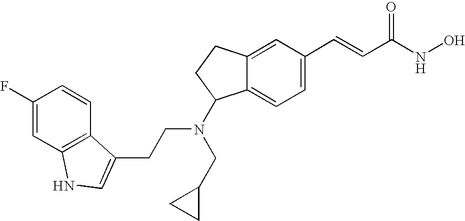 | | | |
| 612 | 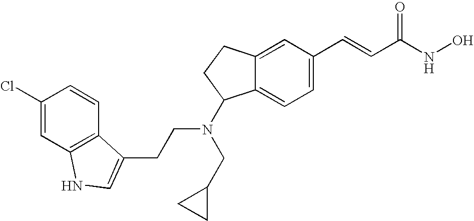 | | | |
| 613 | 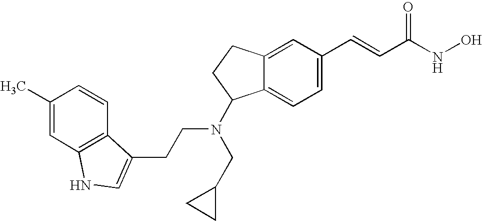 | | | |
| 614 | 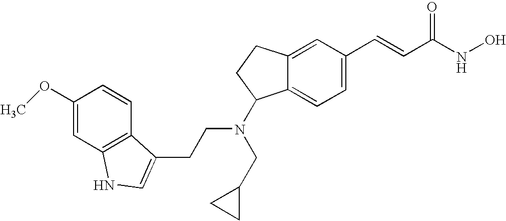 | | | |
| 615 | 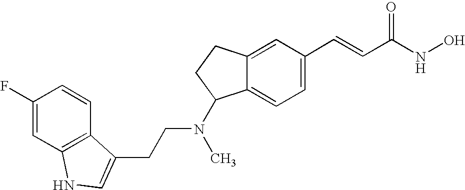 | | | |
| 616 | 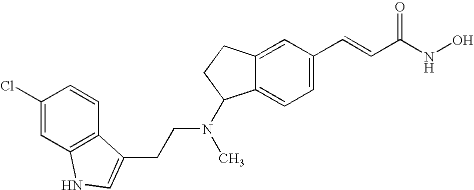 | | | |
| 617 | 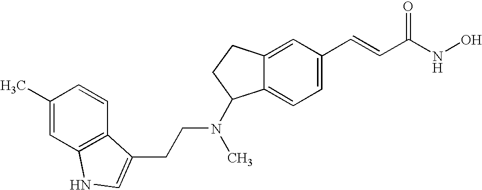 | | | |
| 618 | 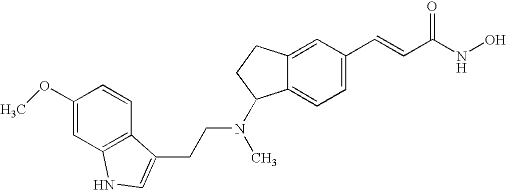 | | | |
| 619 | 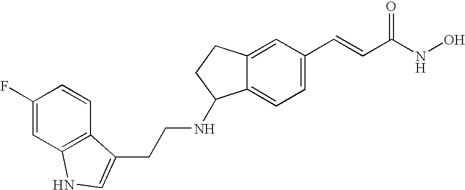 | D, Q60, 619 | 2.02 (A) | 379.9 |
| 620 | 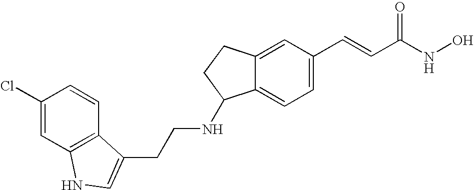 | D, Q59, 620 | 2.14 (A) | 395.9 |
| 621 | 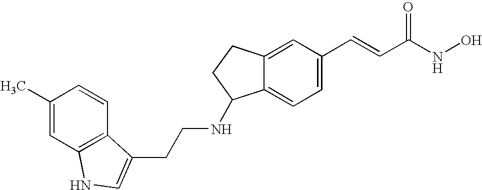 | D, Q54, 621 | 2.00 (A) | 376.1 |
| 622 | 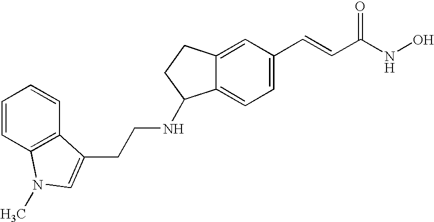 | D, Q58, 622 | 2.07 (A) | 376.1 |
| 623 | 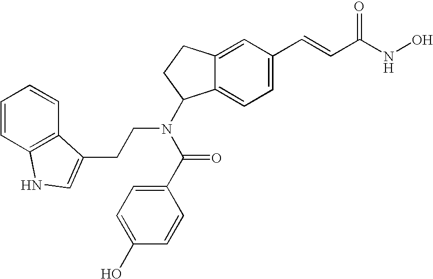 | | | |
| 624 | 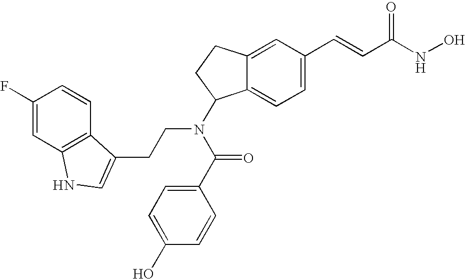 | | | |
| 625 | 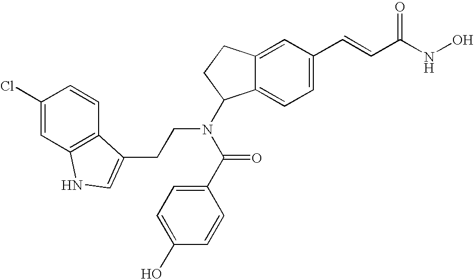 | | | |
| 626 | 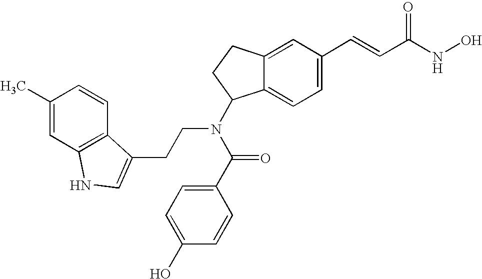 | | | |
| 627 | 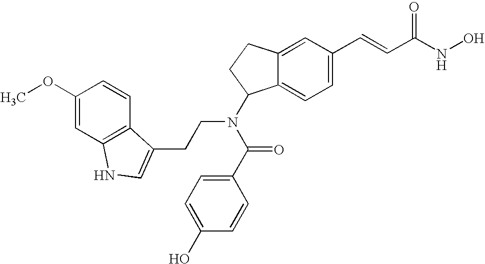 | | | |
| 628 | 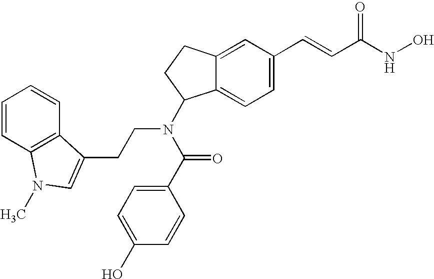 | | | |
| 629 | 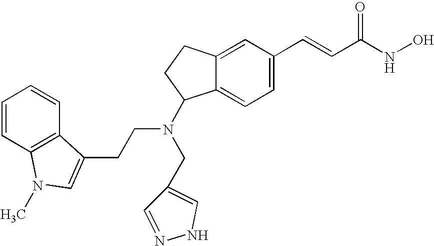 | | | |
| 630 | 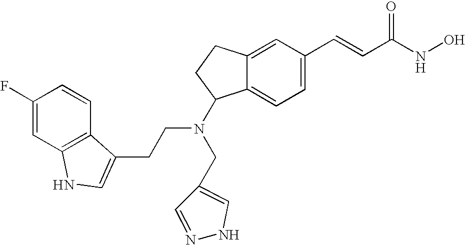 | | | |
| 631 | 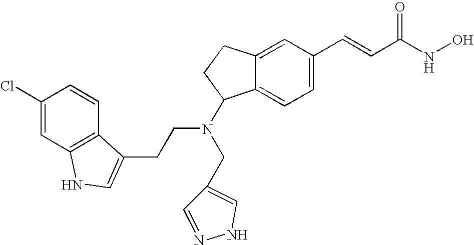 | | | |
| 632 | 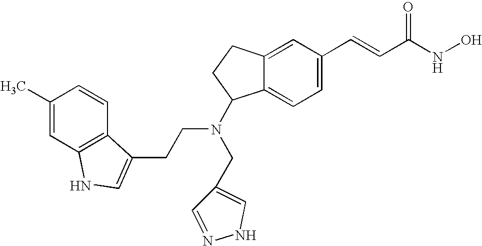 | | | |
| 633 | 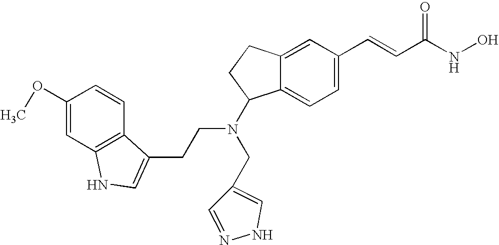 | | | |
| 634 | 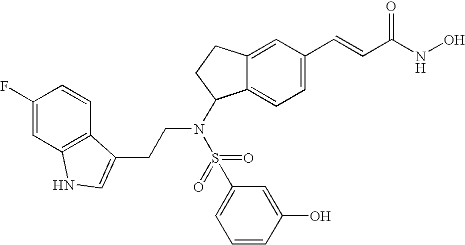 | | | |
| 635 | 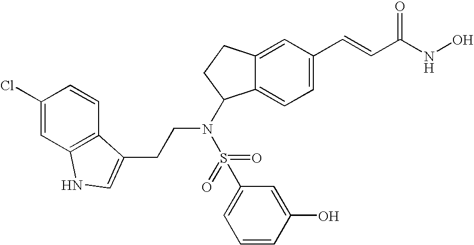 | | | |
| 636 | 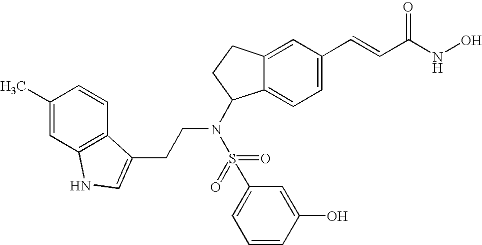 | | | |
| 637 | 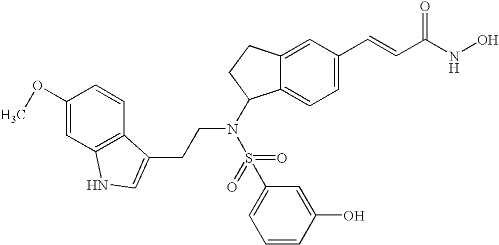 | | | |
| 638 | 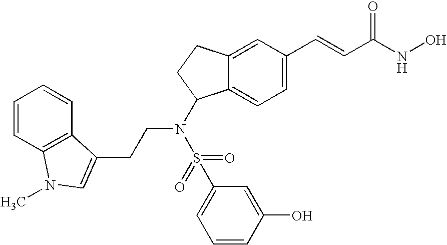 | | | |
| 639 | 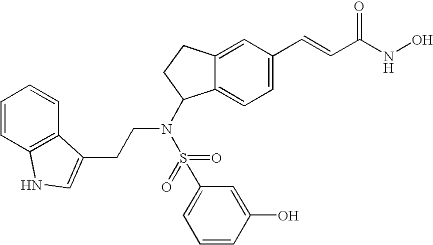 | | | |
| 640 | 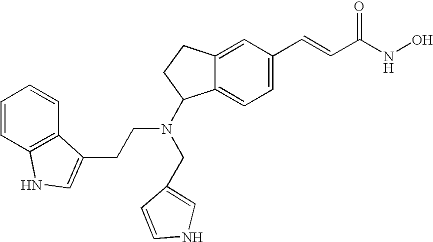 | | | |
| 641 | 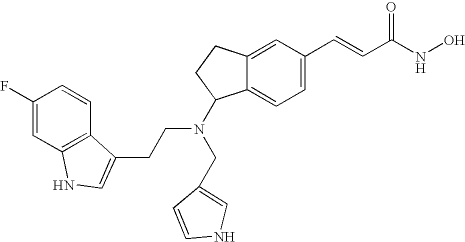 | | | |
| 642 | 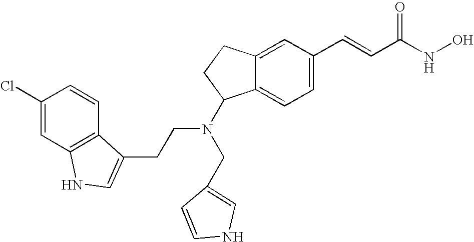 | | | |
| 643 | 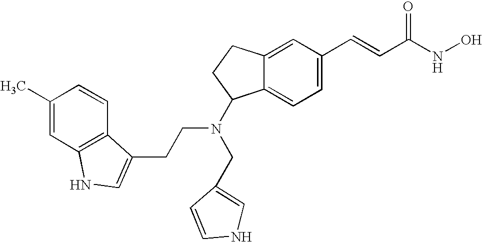 | | | |
| 644 | 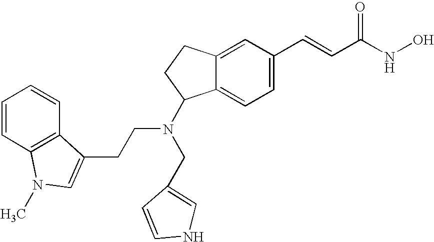 | | | |
| 645 | 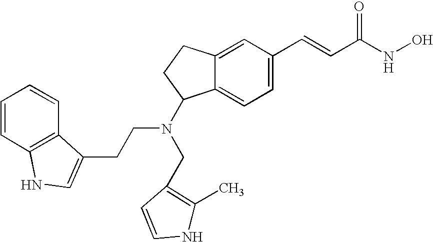 | | | |
| 646 | 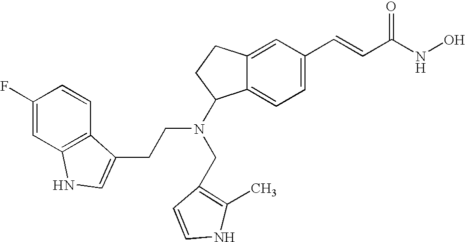 | | | |
| 647 | 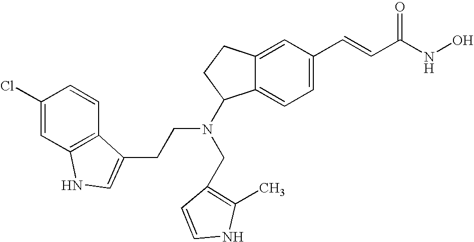 | | | |
| 648 | 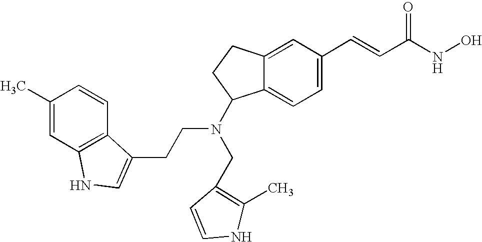 | | | |
| 649 | 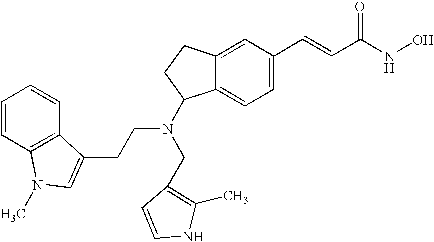 | | | |
| 650 | 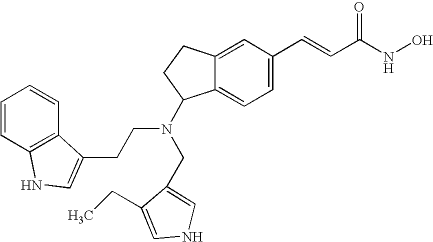 | | | |
| 651 | 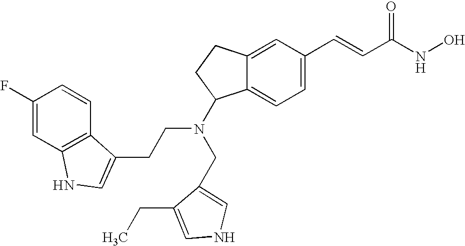 | | | |
| 652 | 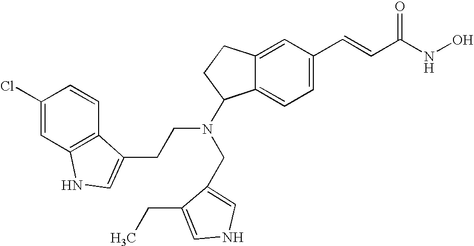 | | | |
| 653 | 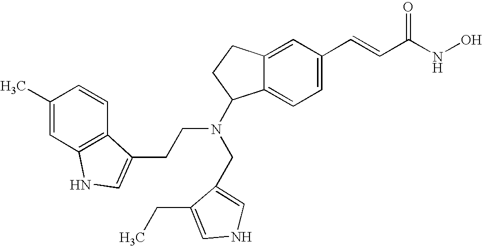 | | | |
| 654 | 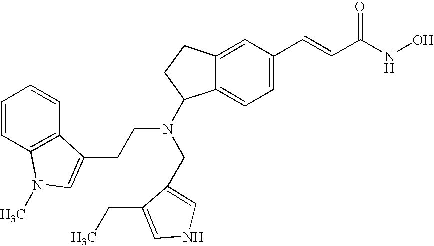 | | | |
| 655 | 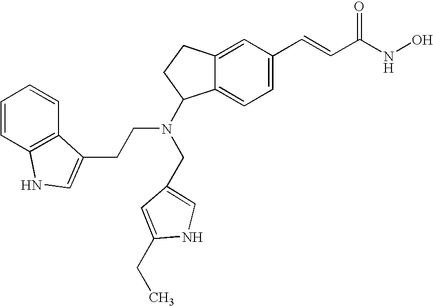 | | | |
| 656 | 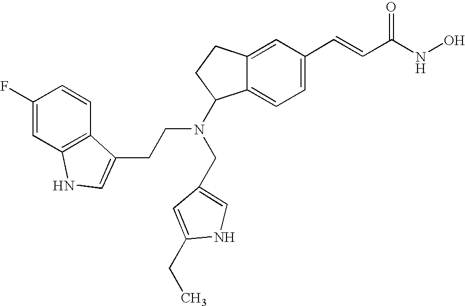 | | | |
| 657 | 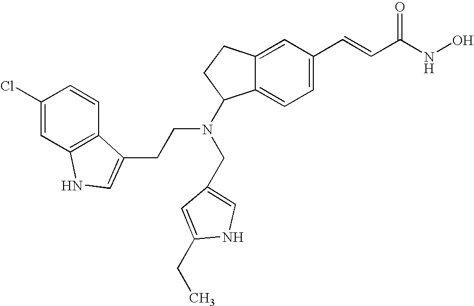 | | | |
| 658 | 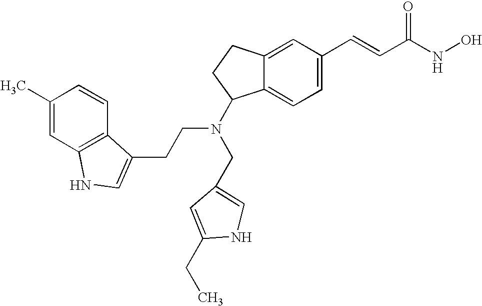 | | | |
| 659 | 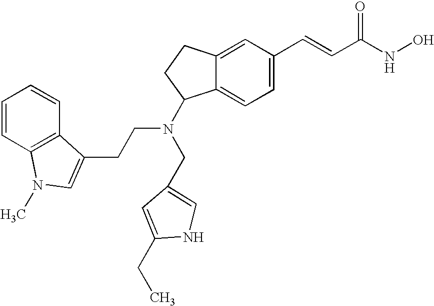 | | | |
| 660 | 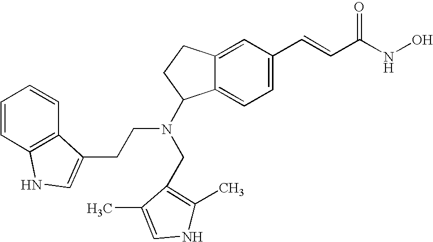 | | | |
| 661 | 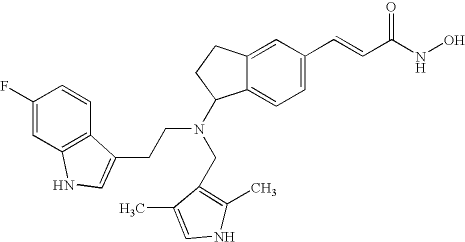 | | | |
| 662 | 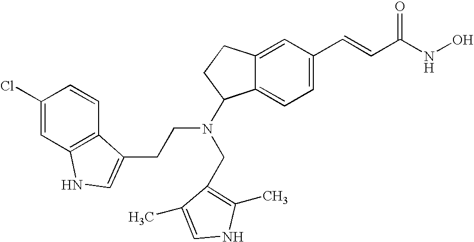 | | | |
| 663 | 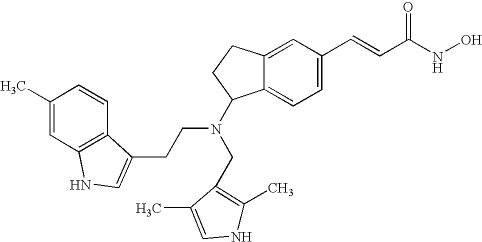 | | | |
| 664 | 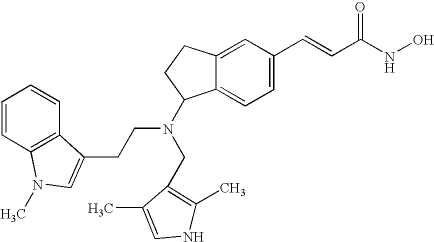 | | | |
| 665 | 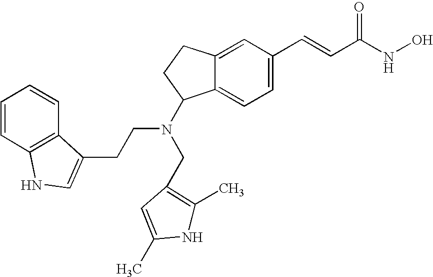 | | | |
| 666 | 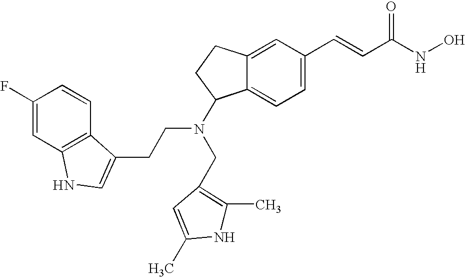 | | | |
| 667 | 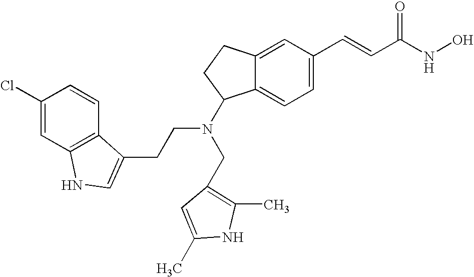 | | | |
| 668 | 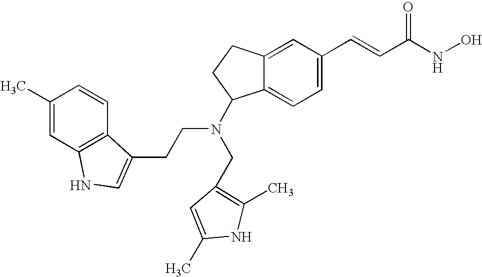 | | | |
| 669 | 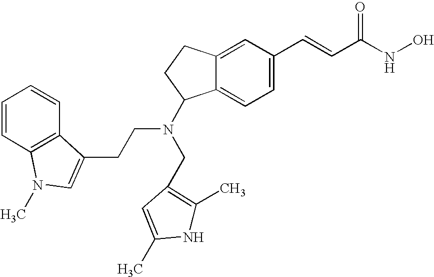 | | | |
| 670 | 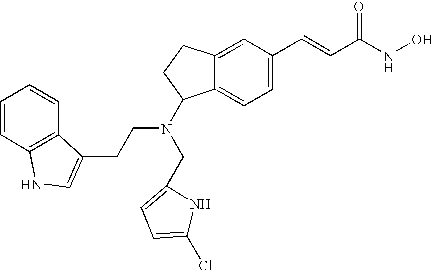 | | | |
| 671 | 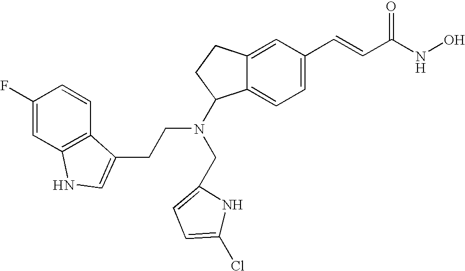 | | | |
| 672 | 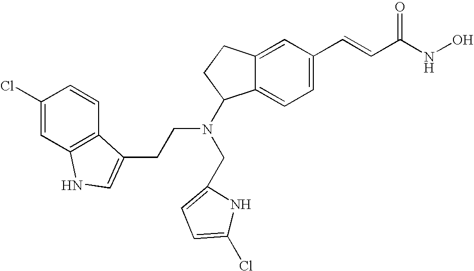 | | | |
| 673 | 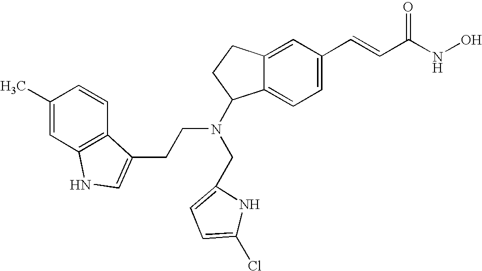 | | | |
| 674 | 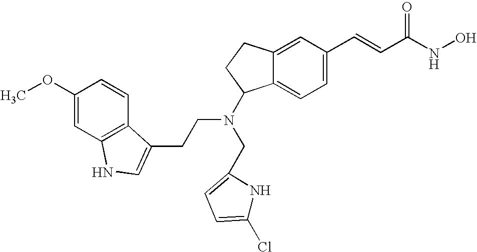 | | | |
| 675 | 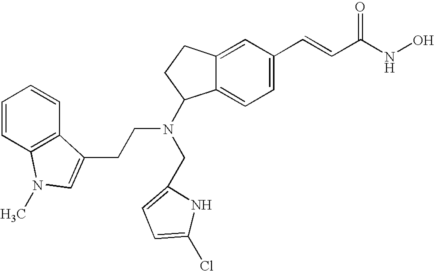 | | | |
| 676 | 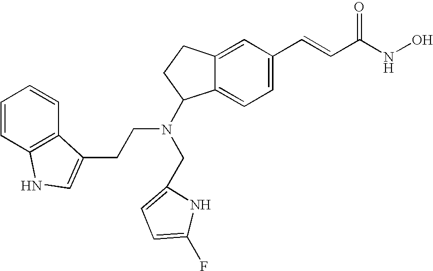 | | | |
| 677 | 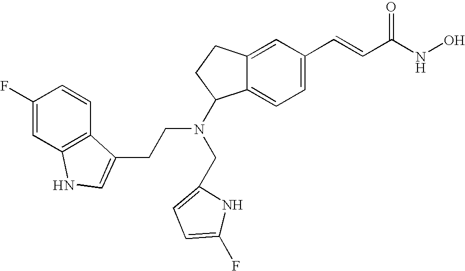 | | | |
| 678 | 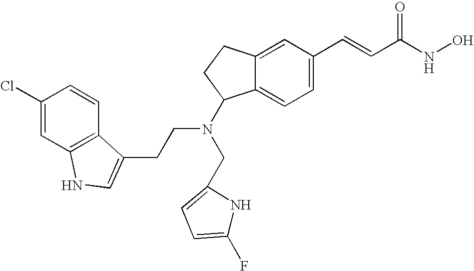 | | | |
| 679 | 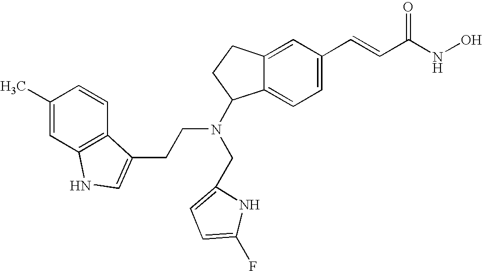 | | | |
| 680 | 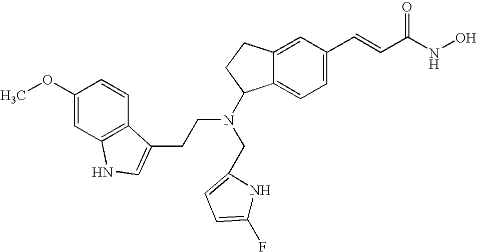 | | | |
| 681 | 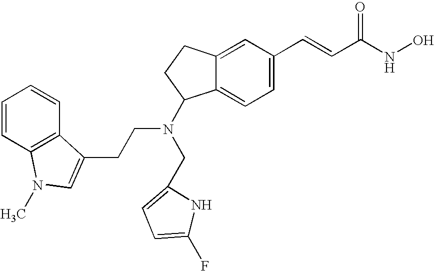 | | | |
| 682 | 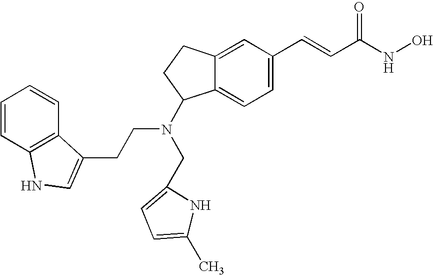 | | | |
| 683 | 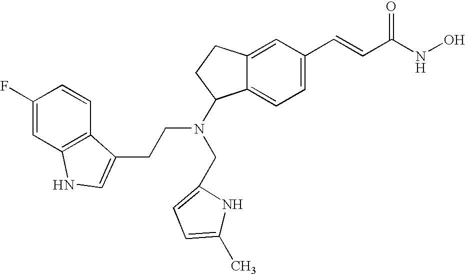 | | | |
| 684 | 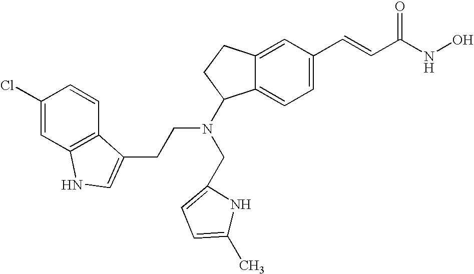 | | | |
| 685 | 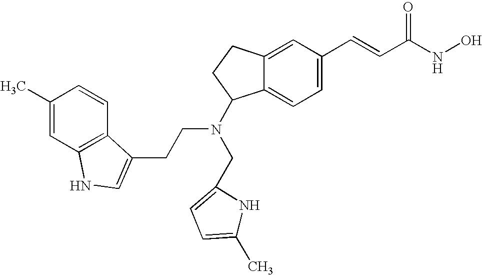 | | | |
| 686 | 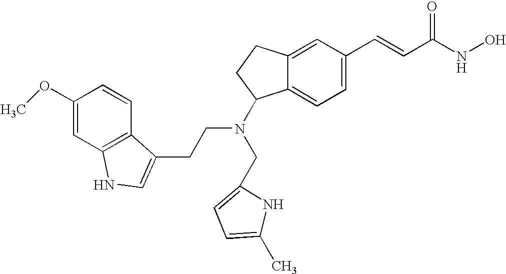 | | | |
| 687 | 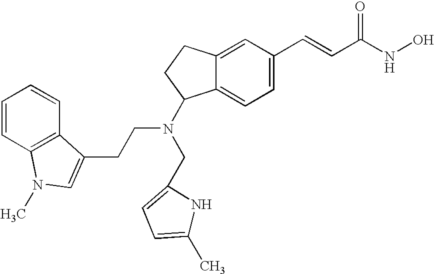 | | | |
| 688 | 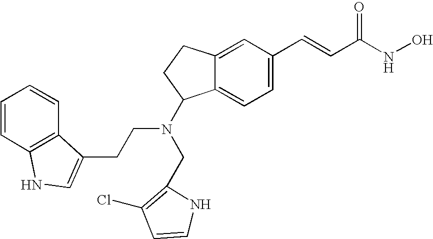 | | | |
| 689 | 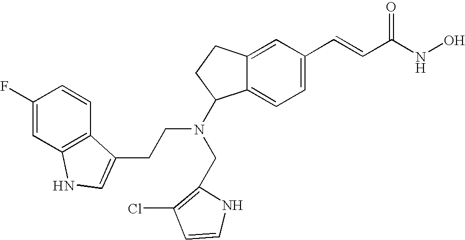 | | | |
| 690 | 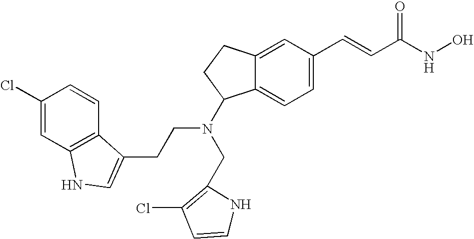 | | | |
| 691 | 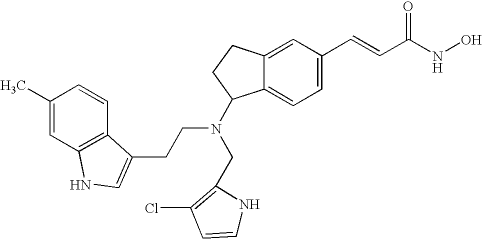 | | | |
| 692 | 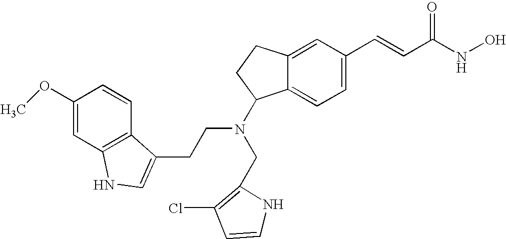 | | | |
| 693 | 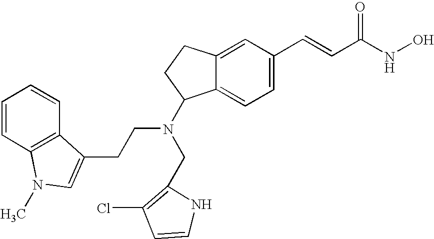 | | | |
| 694 | 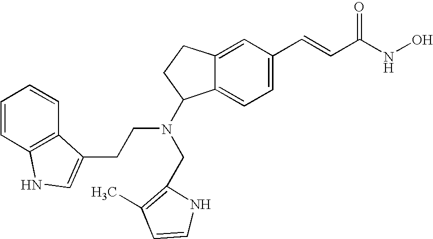 | | | |
| 695 | 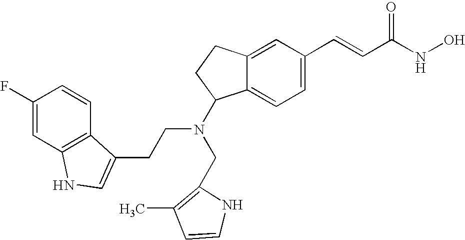 | | | |
| 696 | 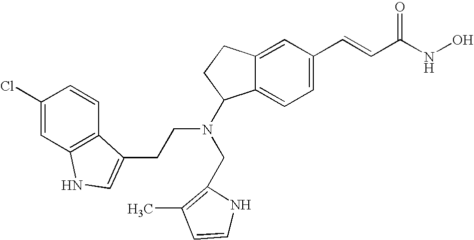 | | | |
| 697 | 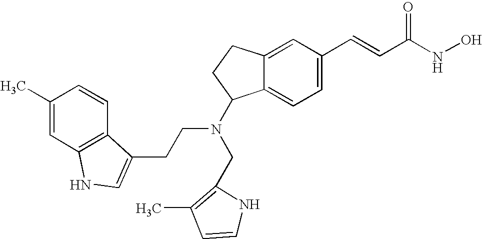 | | | |
| 698 | 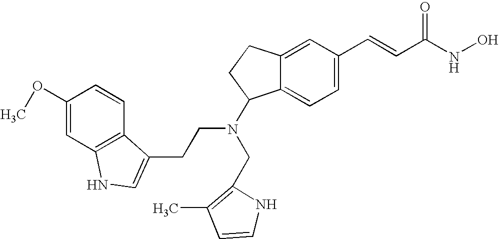 | | | |
| 699 | 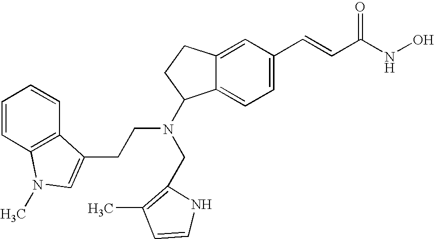 | | | |
| 700 | 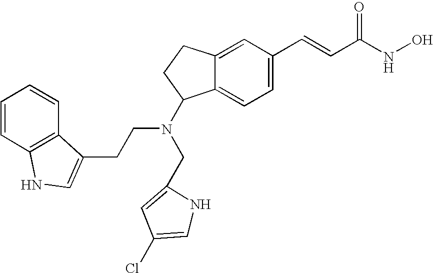 | | | |
| 701 | 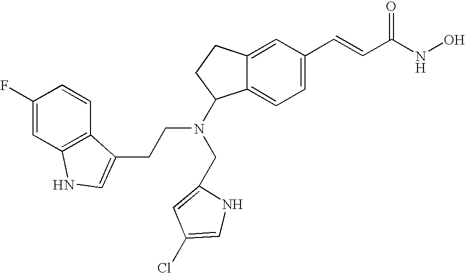 | | | |
| 702 | 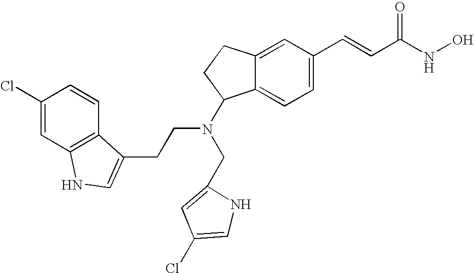 | | | |
| 703 | 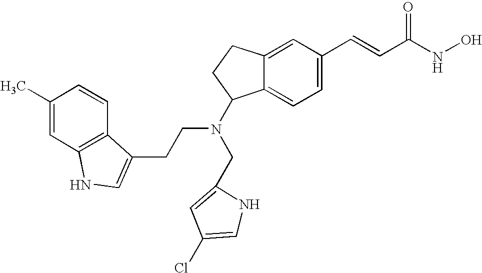 | | | |
| 704 | 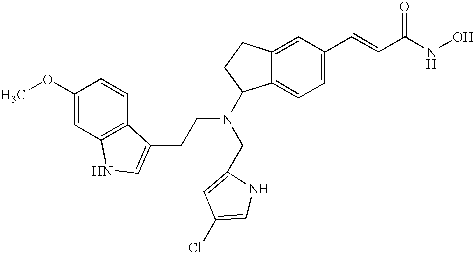 | | | |
| 705 | 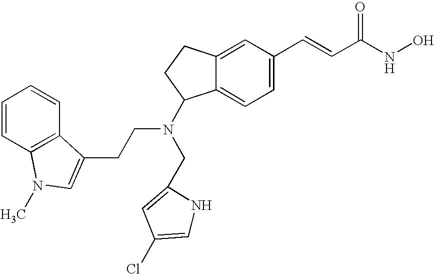 | | | |
| 706 | 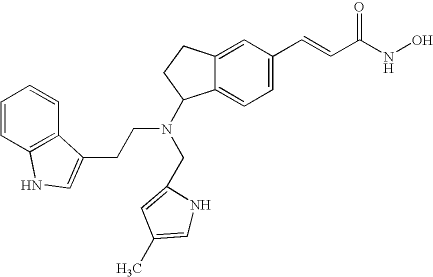 | | | |
| 707 | 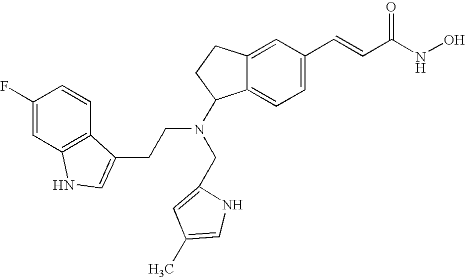 | | | |
| 708 | 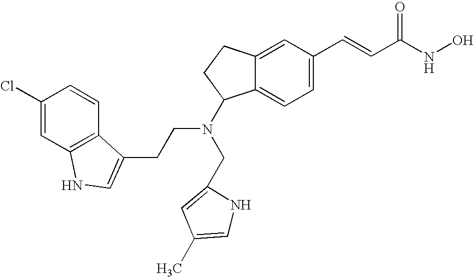 | | | |
| 709 | 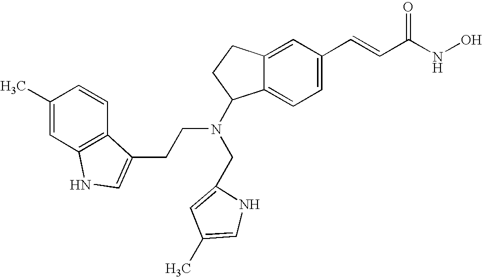 | | | |
| 710 | 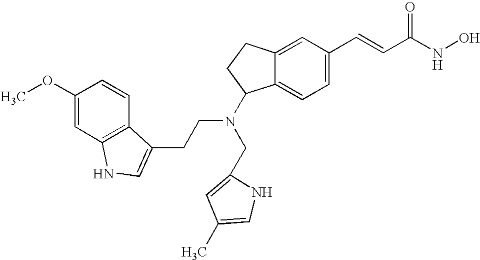 | | | |
| 711 | 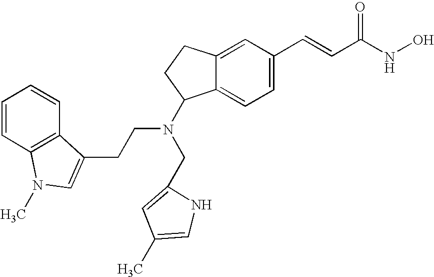 | | | |
| 712 | 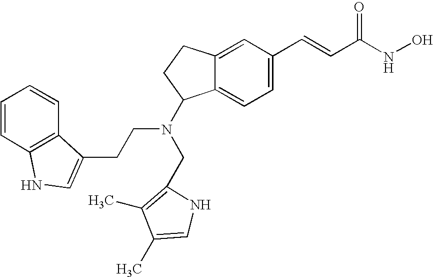 | | | |
| 713 | 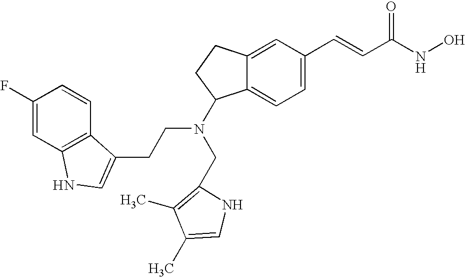 | | | |
| 714 | 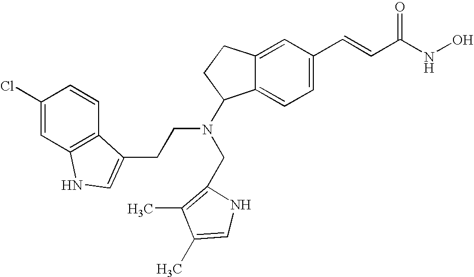 | | | |
| 715 | 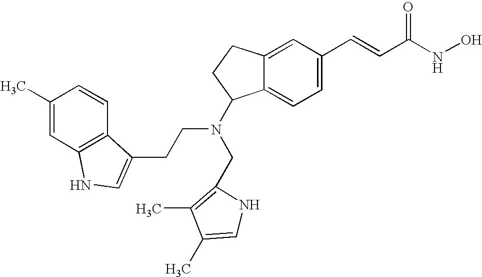 | | | |
| 716 | 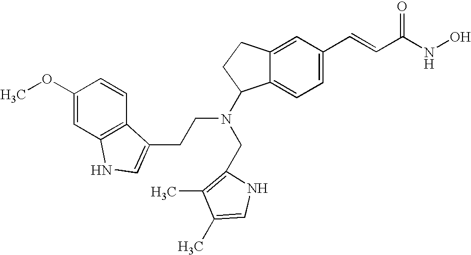 | | | |
| 717 | 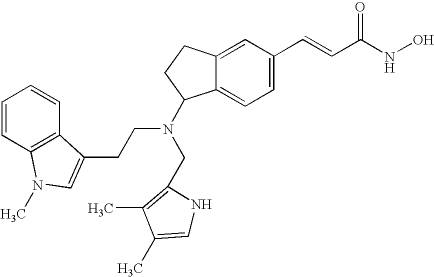 | | | |
| 718 | 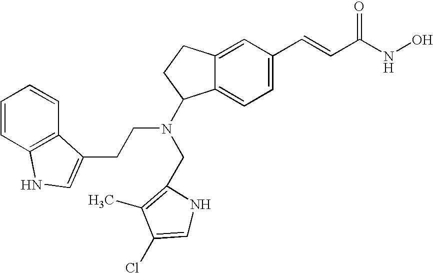 | | | |
| 719 | 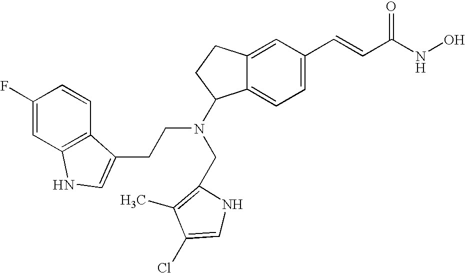 | | | |
| 720 | 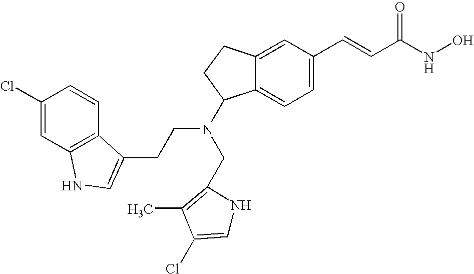 | | | |
| 721 | 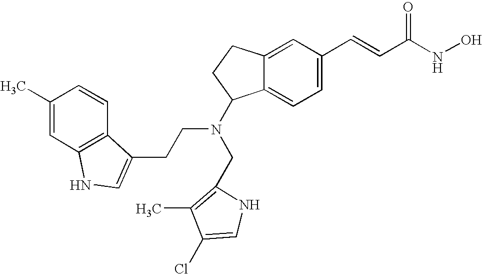 | | | |
| 722 | 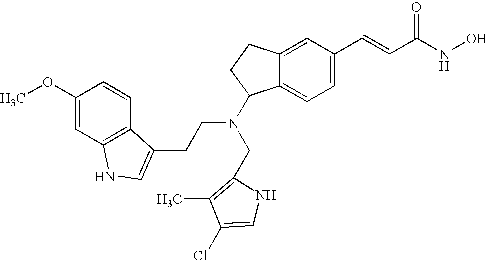 | | | |
| 723 | 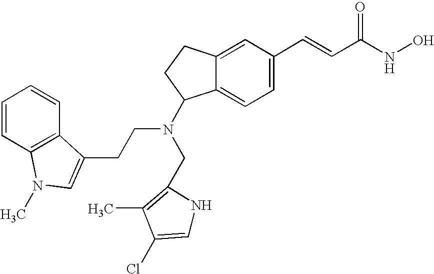 | | | |
| 724 | 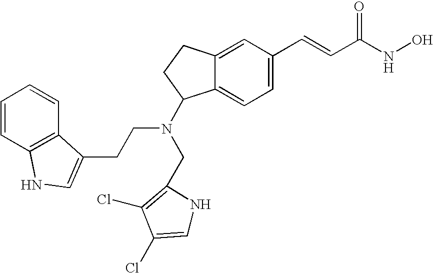 | | | |
| 725 | 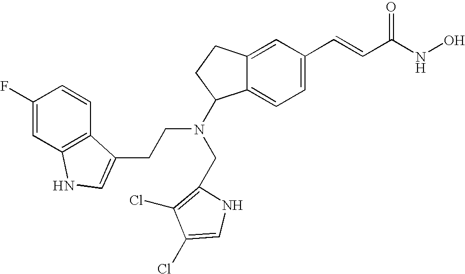 | | | |
| 726 | 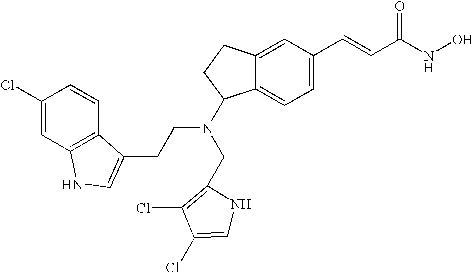 | | | |
| 727 | 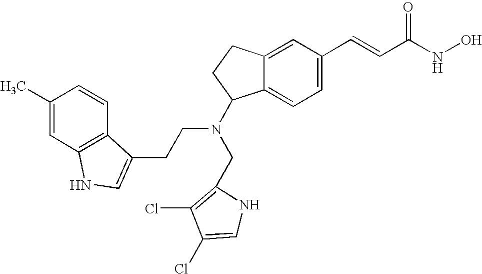 | | | |
| 728 | 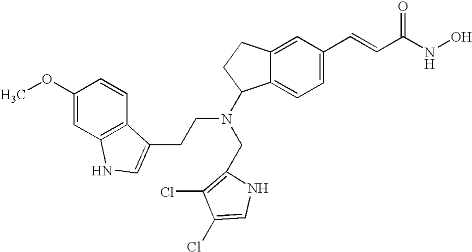 | | | |
| 729 | 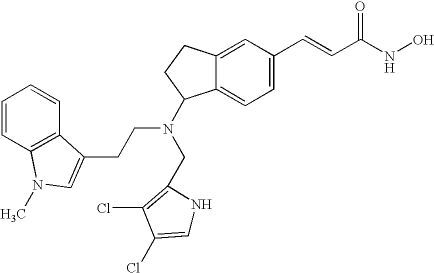 | | | |
| 730 | 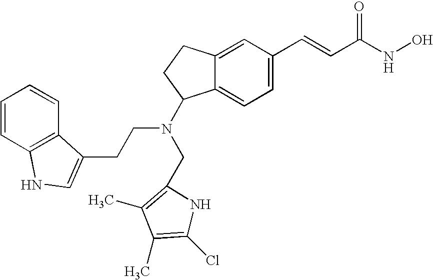 | | | |
| 731 | 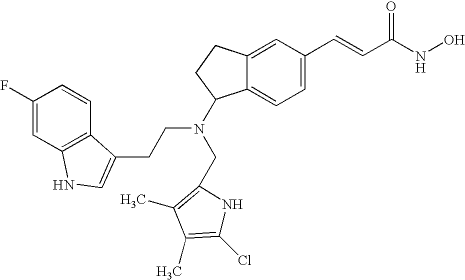 | | | |
| 732 | 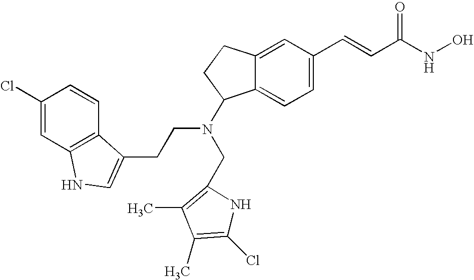 | | | |
| 733 | 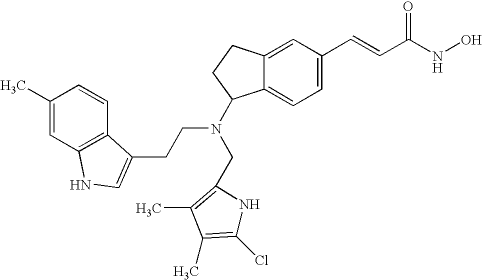 | | | |
| 734 | 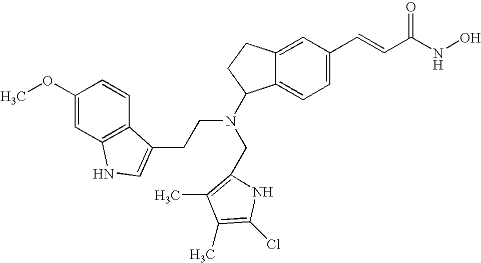 | | | |
| 735 | 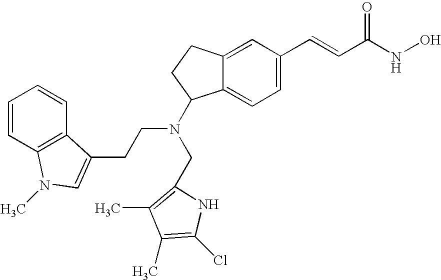 | | | |
| 736 | 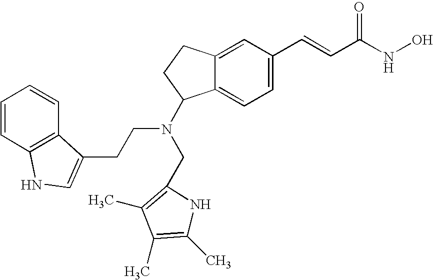 | | | |
| 737 | 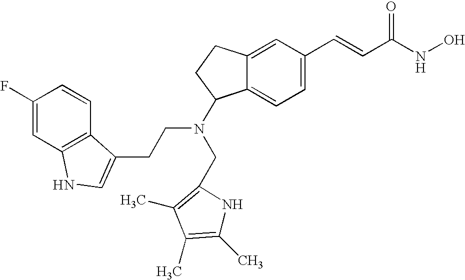 | | | |
| 738 | 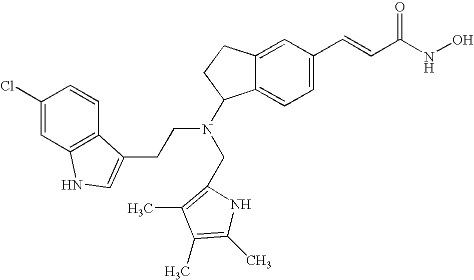 | | | |
| 739 | 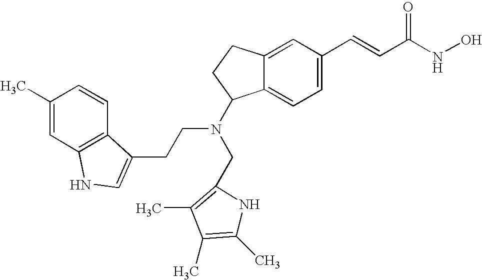 | | | |
| 740 | 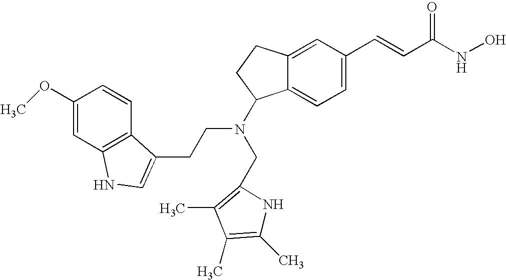 | | | |
| 741 | 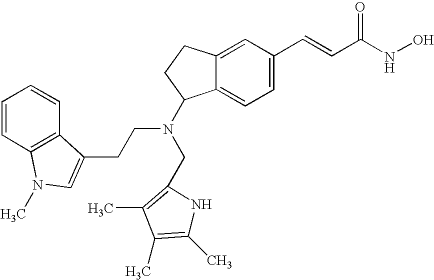 | | | |
| 742 | 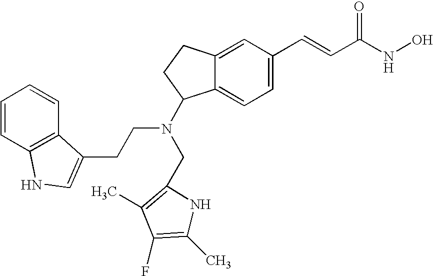 | | | |
| 743 | 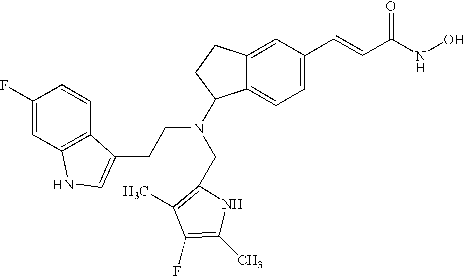 | | | |
| 744 | 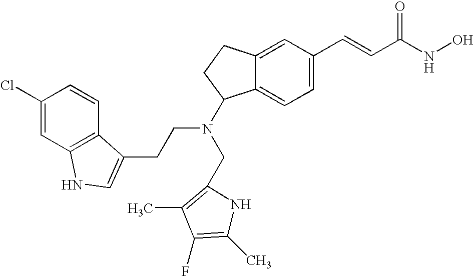 | | | |
| 745 | 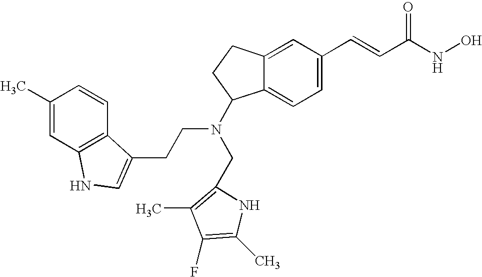 | | | |
| 746 | 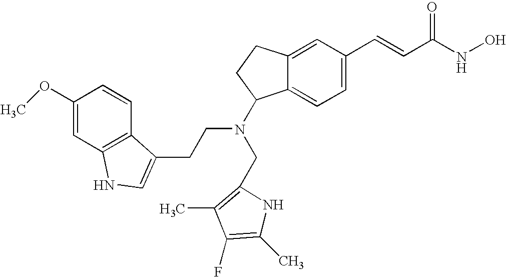 | | | |
| 747 | 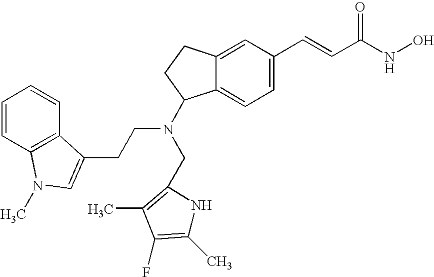 | | | |
| 748 | 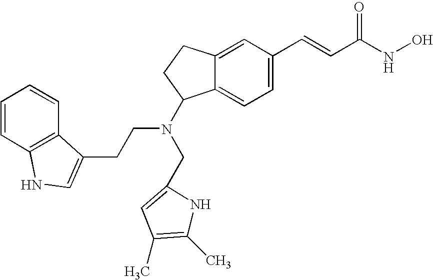 | | | |
| 749 | 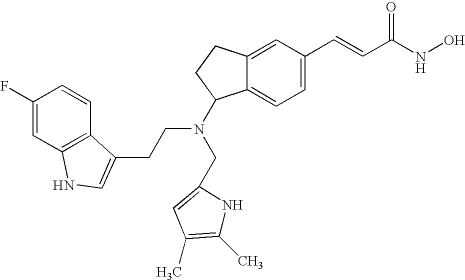 | | | |
| 750 | 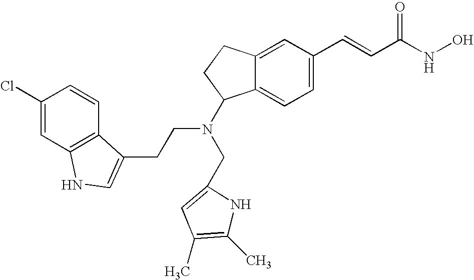 | | | |
| 751 | 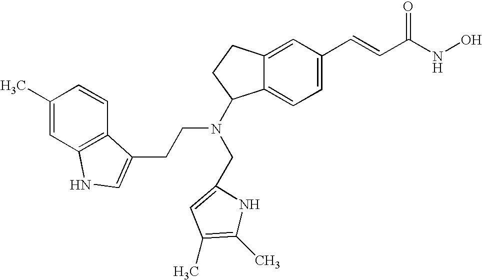 | | | |
| 752 | 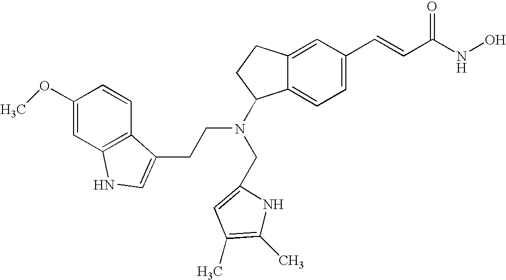 | | | |
| 753 | 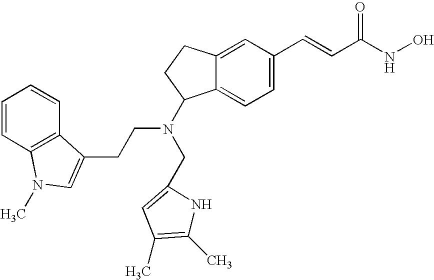 | | | |
| 754 | 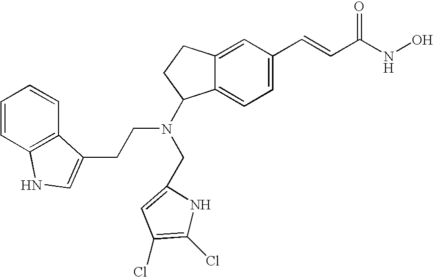 | | | |
| 755 | 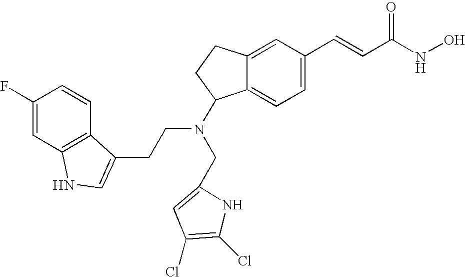 | | | |
| 756 | 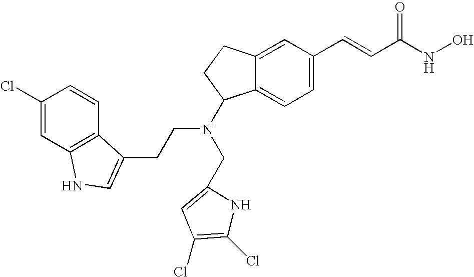 | | | |
| 757 | 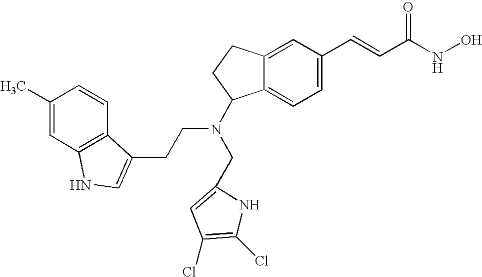 | | | |
| 758 | 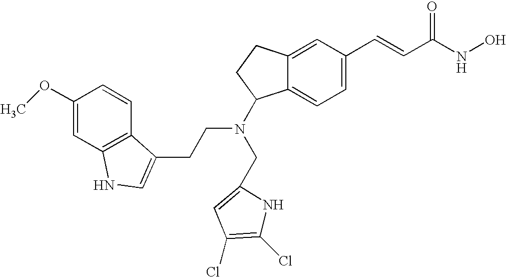 | | | |
| 759 | 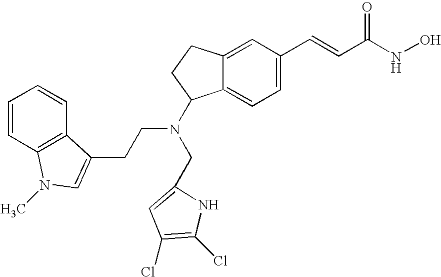 | | | |
| 760 | 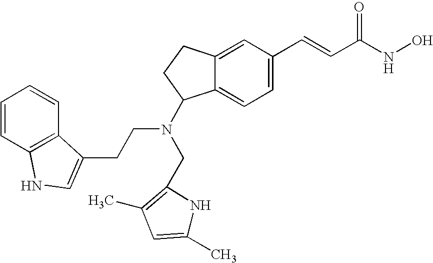 | | | |
| 761 | 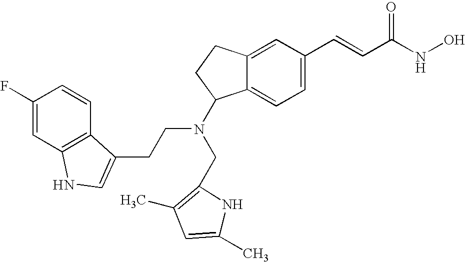 | | | |
| 762 | 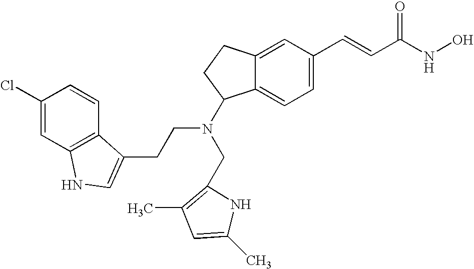 | | | |
| 763 | 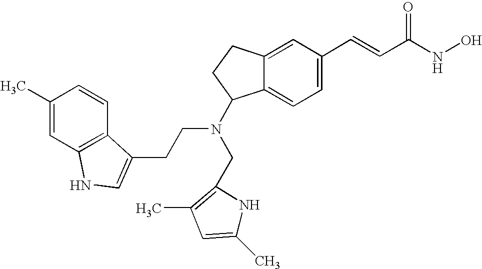 | | | |
| 764 | 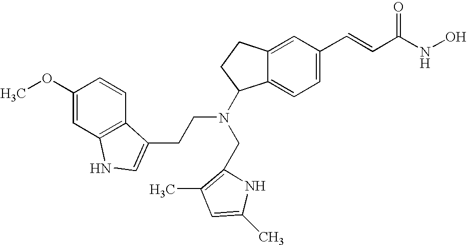 | | | |
| 765 | 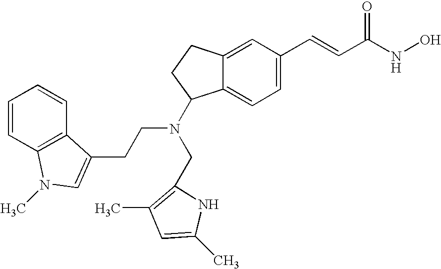 | | | |
| 766 | 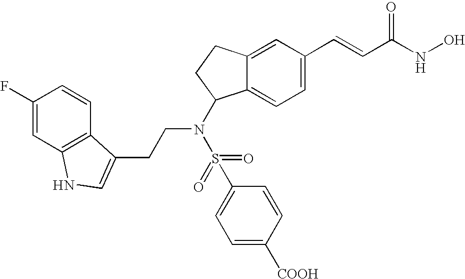 | | | |
| 767 | 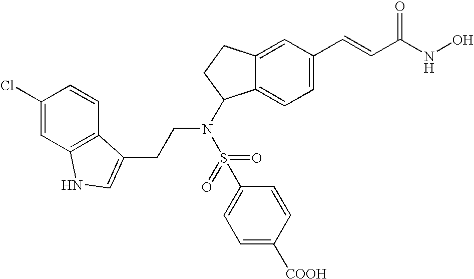 | | | |
| 768 | 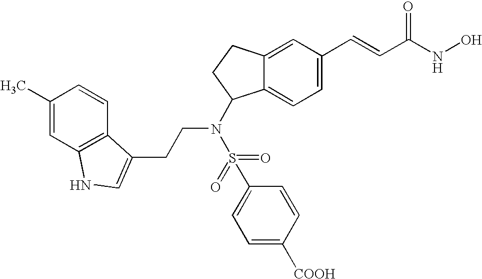 | | | |
| 769 | 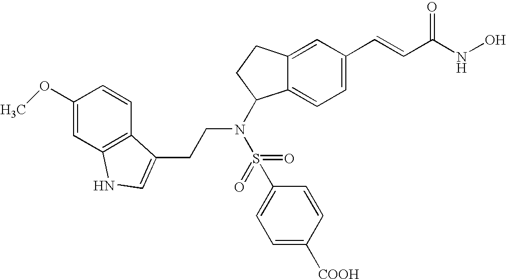 | | | |
| 770 | 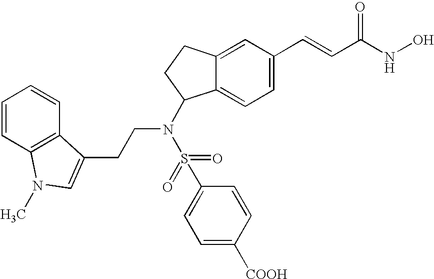 | | | |
| 771 | 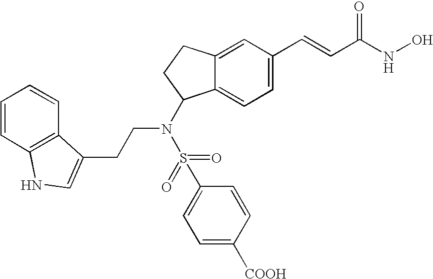 | | | |
| 772 | 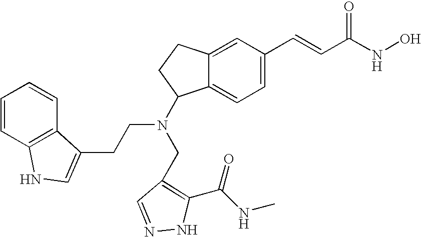 | | | |
| 773 | 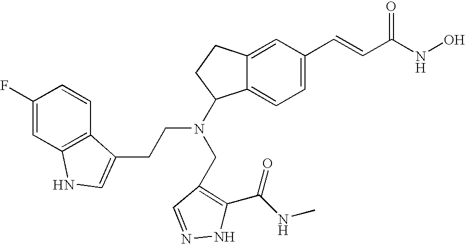 | | | |
| 774 | 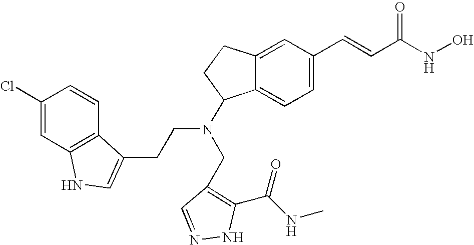 | | | |
| 775 | 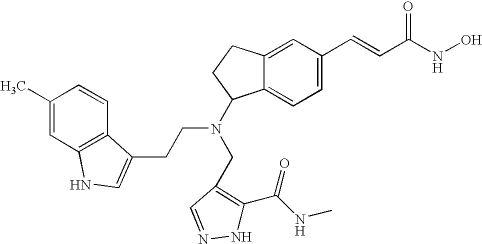 | | | |
| 776 | 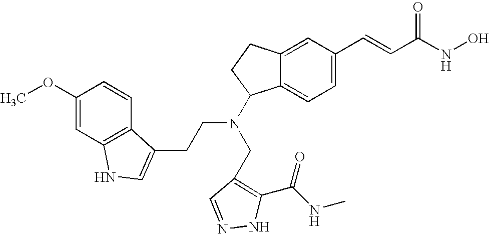 | | | |
| 777 | 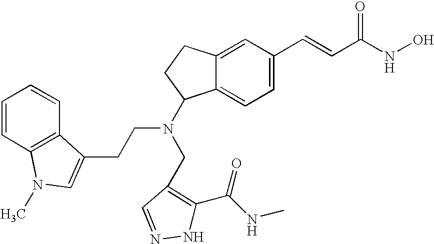 | | | |
| 778 | 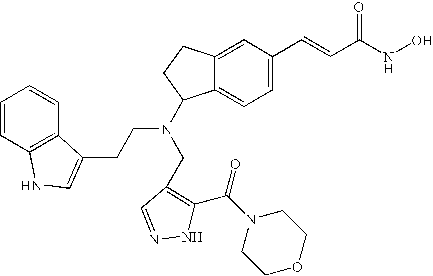 | | | |
| 779 | 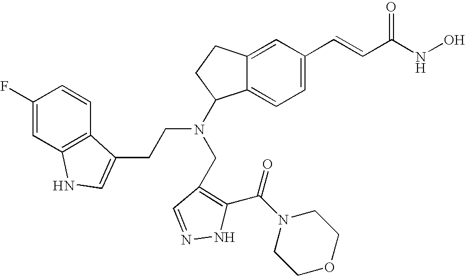 | | | |
| 780 | 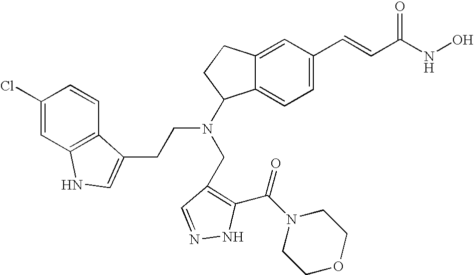 | | | |
| 781 | 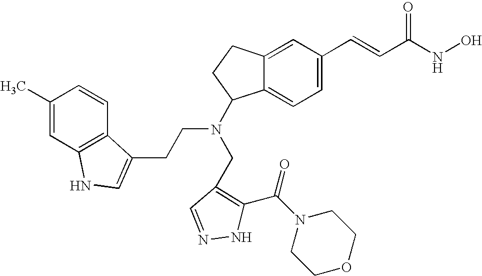 | | | |
| 782 | 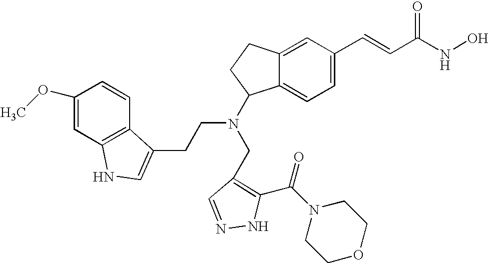 | | | |
| 783 | 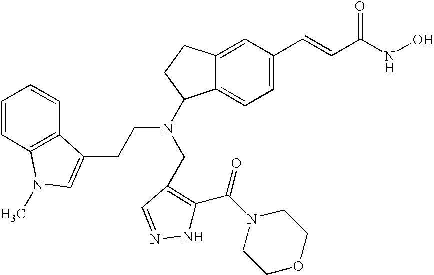 | | | |
| 784 | 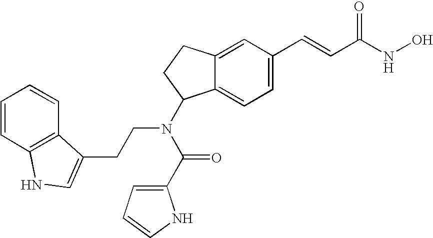 | | | |
| 785 | 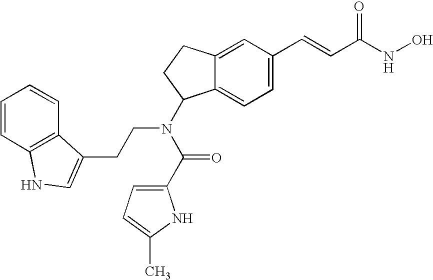 | | | |
| 786 | 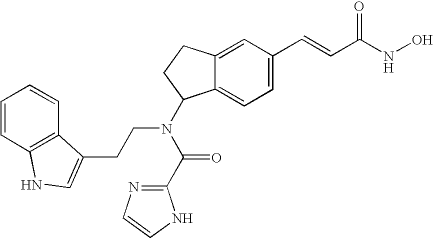 | | | |
| 787 | 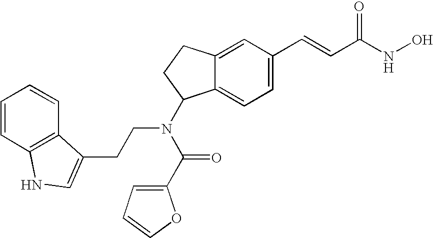 | | | |
| 788 | 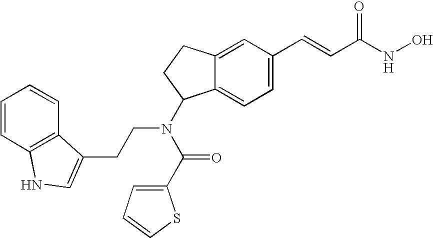 | | | |
| 789 | 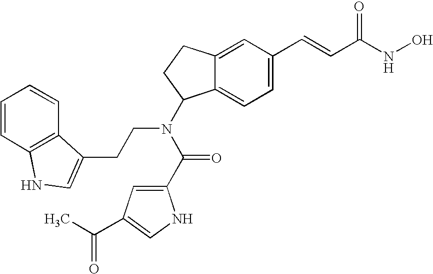 | | | |
| 790 | 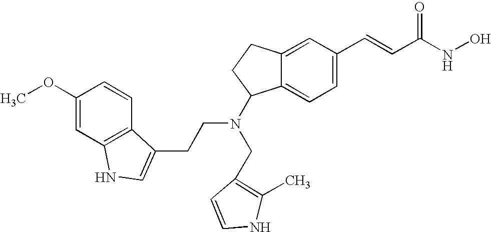 | | | |
| 791 | 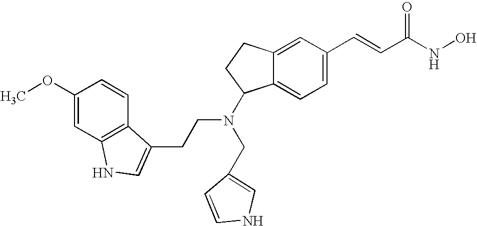 | | | |
| 792 | 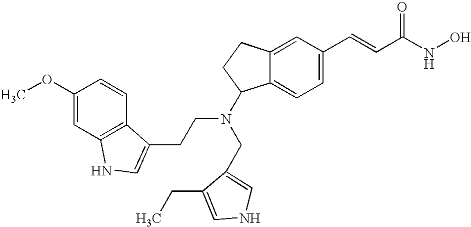 | | | |
| 793 | 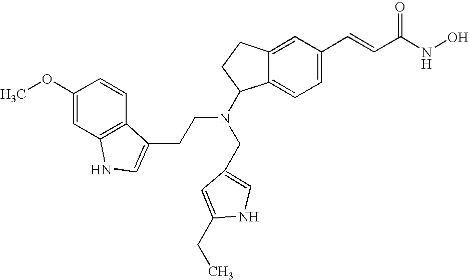 | | | |
| 794 | 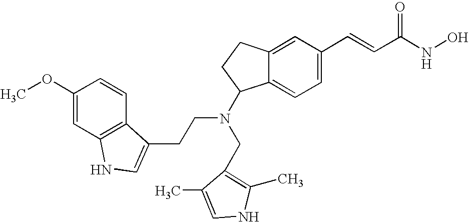 | | | |
| 795 | 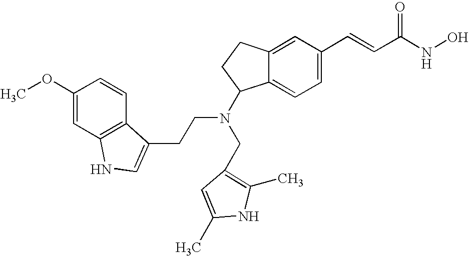 | | | |
| 796 | 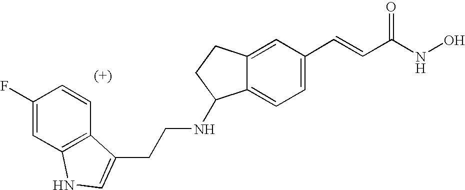 | D, Q60, R91, 796 | 1.93 (A) | 380.0 |
| 797 | 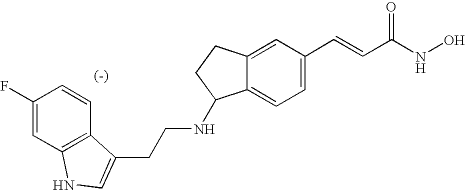 | D, Q60, R92, 797 | 1.92 (A) | 380.0 |
| 798 | 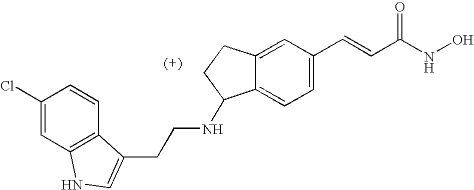 | D, Q59, R93, 798 | 2.04 (A) | 396.0 |
| 799 | 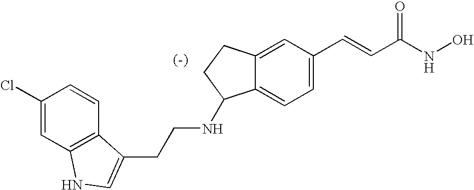 | D, Q59, R94, 799 | 2.06 (A) | 396.0 |
| 800 | 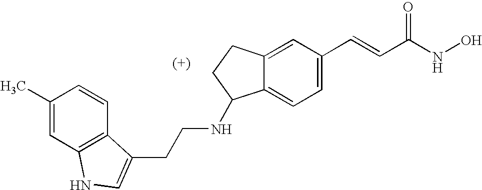 | D, Q54, R95, 800 | 2.03 (A) | 376.1 |
| 801 | 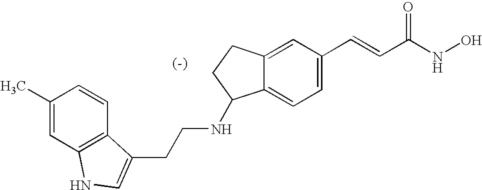 | D, Q54, R96, 801 | 2.03 (A) | 376.0 |
| 802 | 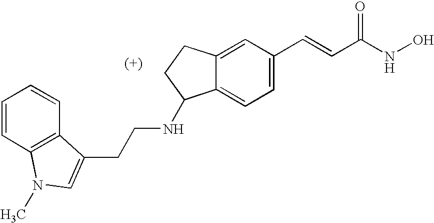 | D, Q58, R97, 802 | 2.01 (A) | 376.1 |
| 803 | 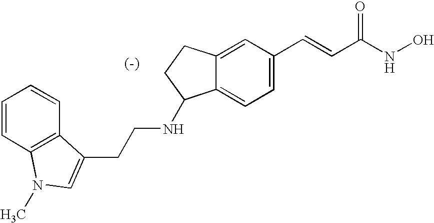 | D, Q58, R98, 803 | 2.00 (A) | 376.0 |
| 804 | 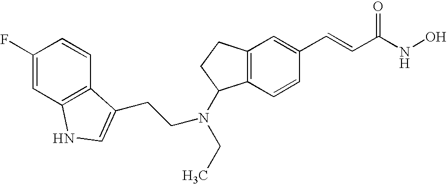 | D, Q60, F170, 804 | 1.92 (A) | 408.0 |
| 805 | 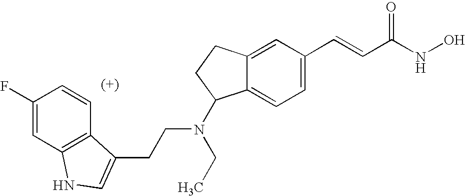 | D, Q60, F170, R99, 805 | 1.90 (A) | 408.0 |
| 806 | 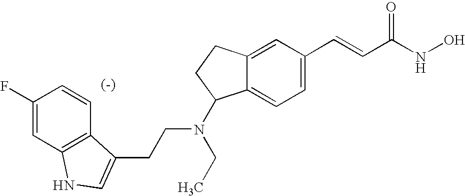 | D, Q60, F170, R100, 806 | 1.90 (A) | 408.0 |
| 807 | 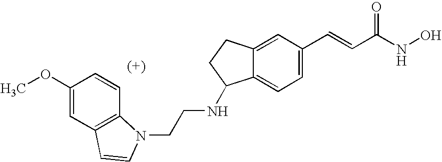 | D, Q52, F101, 807 | 1.97 (A) | 392.0 |
| 808 | 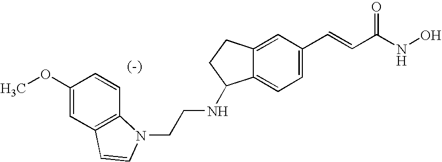 | D, Q52, R102, 808 | 2.20 (A) | 392.0 |

The chemical names for the compounds shown in Table 1 are provided below in Table 2

TABLE 2

| Compound | |
|---|---|
| Example | IUPAC Name |
| 1 | (2E)-N-hydroxy-3-(1-{[(1S)-2-hydroxy-1-(1H-indol-3-ylmethyl)ethyl]amino)- |
| | 2,3-dihydro-1H-inden-5-yl)acrylamide |
| 2 | (2E)-N-hydroxy-3-(1-{(3-hydroxypropyl)[2-(1H-indol-3-yl)ethyl]amino)-2,3- |
| | dihydro-1H-inden-5-yl)acrylamide |
| 3 | (2E)-N-hydroxy-3-((1S)-1-{[2-(1H-indol-3-yl)ethyl]amino}-2,3-dihydro-1H- |
| | inden-5-yl)acrylamide |
| 4 | (2E)-N-hydroxy-3-((1R)-1-{[2-(1H-indol-3-yl)ethyl]amino}-2,3-dihydro-1H- |
| | inden-5-yl)acrylamide |
| 5 | (2E)-N-hydroxy-3-((1R)-1-{(2-hydroxyethyl)[2-(1H-indol-3-yl)ethyl]amino}- |
| | 2,3-dihydro-1H-inden-5-yl)acrylamide |
| 6 | (2E)-N-hydroxy-3-((1S)-1-{(2-hydroxyethyl)[2-(1H-indol-3-yl)ethyl]amino}- |
| | 2,3-dihydro-1H-inden-5-yl)acrylamide |
| 7 | (2E)-N-hydroxy-3-(1-{[(1R)-2-hydroxy-1-(1H-indol-3-ylmethyl)ethyl]amino}- |
| | 2,3-dihydro-1H-inden-5-yl)acrylamide |
| 8 | (2E)-N-hydroxy-3-(1-{[2-(1H-indol-3-yl)ethyl]amino}-2,3-dihydro-1H-inden- |
| | 5-yl)acrylamide |
| 9 | (2E)-N-hydroxy-3-(1-{(2-hydroxyethyl)[2-(1H-indol-3-yl)ethyl]amino}-2,3- |
| | dihydro-1H-inden-5-yl)acrylamide |
| 10 | tert-butyl 3-[2-((tert-butoxycarbonyl){5-[(1E)-3-(hydroxyamino)-3-oxoprop- |
| | 1-en-1-yl]-2,3-dihydro-1H-inden-2-yl}amino)ethyl]-1H-indole-1-carboxylate |
| 11 | (2E)-3-{2-[{2-[1-(2,2-dimethylpropanoyl)-1H-indol-3-yl]ethyl}(2- |
| | hydroxyethyl)amino]-2,3-dihydro-1H-inden-5-yl}-N-hydroxyacrylamide |
| 12 | (2E)-N-hydroxy-3-(5-{[2-(1H-indol-3-yl)ethyl]amino}-5,6,7,8- |
| | tetrahydronaphthalen-2-yl)acrylamide |
| 13 | (2E)-3-(1-{acetyl[2-(1H-indol-3-yl)ethyl]amino}-2,3-dihydro-1H-inden-5-yl)- |
| | N-hydroxyacrylamide |
| 14 | (2E)-N-hydroxy-3-{1-[[2-(1H-indol-3-yl)ethyl](phenylacetyl)amino]-2,3- |
| | dihydro-1H-inden-5-yl}acrylamide |
| 15 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]cyclopentanecarboxamide |
| 16 | (2E)-N-hydroxy-3-((1S)-1-{[(1S)-2-hydroxy-1-(1H-indol-3- |
| | ylmethyl)ethyl]amino}-2,3-dihydro-1H-inden-5-yl)acrylamide |
| 17 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]benzamide |
| 18 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]cyclohexanecarboxamide |
| 19 | (2E)-3-(1-{[(ethylamino)carbonyl][2-(1H-indol-3-yl)ethyl]amino}-2,3- |
| | dihydro-1H-inden-5-yl)-N-hydroxyacrylamide |
| 20 | (2E)-3-(1-{[(tert-butylamino)carbonyl][2-(1H-indol-3-yl)ethyl]amino}-2,3- |
| | dihydro-1H-inden-5-yl)-N-hydroxyacrylamide |
| 21 | (2E)-3-(1-{[(benzylamino)carbonyl][2-(1H-indol-3-yl)ethyl]amino}-2,3- |
| | dihydro-1H-inden-5-yl)-N-hydroxyacrylamide |
| 22 | (2E)-N-hydroxy-3-[1-([2-(1H-indol-3-yl)ethyl]{[(2- |
| | phenylethyl)amino]carbonyl}amino)-2,3-dihydro-1H-inden-5-yl]acrylamide |
| 23 | (2E)-N-hydroxy-3-{1-[[2-(1H-indol-3-yl)ethyl](phenylsulfonyl)amino]-2,3- |
| | dihydro-1H-inden-5-yl}acrylamide |
| 24 | (2E)-N-hydroxy-3-{1-[[2-(1H-indol-3-yl)ethyl](methylsulfonyl)amino]-2,3- |
| | dihydro-1H-inden-5-yl}acrylamide |
| 25 | (2E)-N-hydroxy-3-((1S)-1-{(3-hydroxypropyl)[2-(1H-indol-3-yl)ethyl]amino}- |
| | 2,3-dihydro-1H-inden-5-yl)acrylamide |
| 26 | (2E)-N-hydroxy-3-((1R)-1-{(3-hydroxypropyl)[2-(1H-indol-3-yl)ethyl]amino}- |
| | 2,3-dihydro-1H-inden-5-yl)acrylamide |
| 27 | (2E)-N-hydroxy-3-{1-[[2-(1H-indol-3-yl)ethyl](2-methoxyethyl)amino]-2,3- |
| | dihydro-1H-inden-5-yl}acrylamide |
| 28 | tert-butyl (2-{{5-[(1E)-3-(hydroxyamino)-3-oxoprop-1-en-1-yl]-2,3-dihydro- |
| | 1H-inden-1-yl}[2-(1H-indol-3-yl)ethyl]amino}ethyl)carbamate |
| 29 | tert-butyl N-{5-[(1E)-3-(hydroxyamino)-3-oxoprop-1-en-1-yl]-2,3-dihydro- |
| | 1H-inden-1-yl}-N-[2-(1H-indol-3-yl)ethyl]glycinate |
| 30 | (2E)-N-hydroxy-3-{1-[(pyridin-3-ylmethyl)amino]-2,3-dihydro-1H-inden-5- |
| | yl}acrylamide |
| 31 | (2E)-N-hydroxy-3-{1-[(2-pyridin-3-ylethyl)amino]-2,3-dihydro-1H-inden-5- |
| | yl}acrylamide |
| 32 | (2E)-N-hydroxy-3-{1-[(2-hydroxyethyl)(2-methylbenzyl)amino]-2,3-dihydro- |
| | 1H-inden-5-yl}acrylamide |
| 33 | (2E)-N-hydroxy-3-{1-[[2-(1H-indol-3-yl)ethyl](methyl)amino]-2,3-dihydro- |
| | 1H-inden-5-yl}acrylamide |
| 34 | (2E)-N-hydroxy-3-(1-{(2-hydroxyethyl)[2-(1-methyl-1H-indol-3- |
| | yl)ethyl]amino}-2,3-dihydro-1H-inden-5-yl)acrylamide |
| 35 | (2E)-3-{1-[[2-(1-ethyl-1H-indol-3-yl)ethyl](2-hydroxyethyl)amino]-2,3- |
| | dihydro-1H-inden-5-yl}-N-hydroxyacrylamide |
| 36 | (2E)-N-hydroxy-3-[1-((2-hydroxyethyl){2-[1-(2-hydroxyethyl)-1H-indol-3- |
| | yl]ethyl}amino)-2,3-dihydro-1H-inden-5-yl]acrylamide |
| 37 | (2E)-N-hydroxy-3-(5-{(2-hydroxyethyl)[2-(1H-indol-3-yl)ethyl]amino}- |
| | 5,6,7,8-tetrahydronaphthalen-2-yl)acrylamide |
| 38 | (2E)-N-hydroxy-3-((5R)-5-{(2-hydroxyethyl)[2-(1H-indol-3-yl)ethyl]amino}- |
| | 5,6,7,8-tetrahydronaphthalen-2-yl)acrylamide |
| 39 | (2E)-N-hydroxy-3-((5S)-5-{(2-hydroxyethyl)[2-(1H-indol-3-yl)ethyl]amino}- |
| | 5,6,7,8-tetrahydronaphthalen-2-yl)acrylamide |
| 40 | (2E)-3-(1-{[(1R)-1-benzyl-2-hydroxyethyl]amino}-2,3-dihydro-1H-inden-5- |
| | yl)-N-hydroxyacrylamide |
| 41 | (2E)-3-(1-{[(1S)-1-benzyl-2-hydroxyethyl]amino}-2,3-dihydro-1H-inden-5- |
| | yl)-N-hydroxyacrylamide |
| 42 | (2E)-N-hydroxy-3-(1-{[1-(hydroxymethyl)-2-methylbutyl]amino}-2,3- |
| | dihydro-1H-inden-5-yl)acrylamide |
| 43 | (2E)-N-hydroxy-3-(2-{[2-(1H-indol-3-yl)ethyl]amino}-2,3-dihydro-1H-inden- |
| | 5-yl)acrylamide |
| 44 | (2E)-N-hydroxy-3-(2-{(2-hydroxyethyl)[2-(1H-indol-3-yl)ethyl]amino}-2,3- |
| | dihydro-1H-inden-5-yl)acrylamide trifluoroacetate (salt) |
| 45 | (2E)-3-(1-{(2-aminoethyl)[2-(1H-indol-3-yl)ethyl]amino}-2,3-dihydro-1H- |
| | inden-5-yl)-N-hydroxyacrylamide |
| 46 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]glycine |
| 47 | (2E)-N-hydroxy-3-(1-{[3-(1H-imidazol-1-yl)propanoyl][2-(1H-indol-3- |
| | yl)ethyl]amino}-2,3-dihydro-1H-inden-5-yl)acrylamide |
| 48 | (2E)-N-hydroxy-3-(1-{[2-(1H-indol-3-yl)ethyl][3-(4-methylpiperazin-1- |
| | yl)propanoyl]amino}-2,3-dihydro-1H-inden-5-yl)acrylamide |
| 49 | (2E)-N-hydroxy-3-{1-[[2-(1H-indol-3-yl)ethyl](3-morpholin-4- |
| | ylpropanoyl)amino]-2,3-dihydro-1H-inden-5-yl}acrylamide |
| 50 | (2E)-3-(1-{(benzylsulfonyl)[2-(1H-indol-3-yl)ethyl]amino}-2,3-dihydro-1H- |
| | inden-5-yl)-N-hydroxyacrylamide |
| 51 | (2E)-N-hydroxy-3-(1-{(2-hydroxyethyl)[2-(1H-indol-3-yl)ethyl]amino}-2,3- |
| | dihydro-1H-inden-5-yl)but-2-enamide |
| 52 | (2Z)-3-chloro-N-hydroxy-3-(1-{(2-hydroxyethyl)[2-(1H-indol-3- |
| | yl)ethyl]amino}-2,3-dihydro-1H-inden-5-yl)acrylamide |
| 53 | N-hydroxy-3-(1-{(2-hydroxyethyl)[2-(1H-indol-3-yl)ethyl]amino}-2,3- |
| | dihydro-1H-inden-5-yl)propanamide |
| 54 | 3-(6-chloro-1-{(2-hydroxyethyl)[2-(1H-indol-3-yl)ethyl]amino}-2,3-dihydro- |
| | 1H-inden-5-yl)-N-hydroxypropanamide |
| 55 | 3-(4-chloro-1-{(2-hydroxyethyl)[2-(1H-indol-3-yl)ethyl]amino}-2,3-dihydro- |
| | 1H-inden-5-yl)-N-hydroxypropanamide |
| 56 | N-hydroxy-3-(1-{(2-hydroxyethyl)[2-(1H-indol-3-yl)ethyl]amino}-6-methoxy- |
| | 2,3-dihydro-1H-inden-5-yl)propanamide |
| 57 | 3-(4,6-dichloro-1-{(2-hydroxyethyl)[2-(1H-indol-3-yl)ethyl]amino}-2,3- |
| | dihydro-1H-inden-5-yl)-N-hydroxypropanamide |
| 58 | N-hydroxy-3-(1-{(2-hydroxyethyl)[2-(1H-indol-3-yl)ethyl]amino}-4-methyl- |
| | 2,3-dihydro-1H-inden-5-yl)propanamide |
| 59 | N-hydroxy-3-(1-{(2-hydroxyethyl)[2-(1H-indol-3-yl)ethyl]amino}-6-methyl- |
| | 2,3-dihydro-1H-inden-5-yl)propanamide |
| 60 | N-hydroxy-3-[1-{(2-hydroxyethyl)[2-(1H-indol-3-yl)ethyl]amino}-6- |
| | (trifluoromethyl)-2,3-dihydro-1H-inden-5-yl]propanamide |
| 61 | N-hydroxy-3-(1-{(2-hydroxyethyl)[2-(1H-indol-3-yl)ethyl]amino}-6-nitro-2,3- |
| | dihydro-1H-inden-5-yl)propanamide |
| 62 | 3-(6-amino-1-{(2-hydroxyethyl)[2-(1H-indol-3-yl)ethyl]amino}-2,3-dihydro- |
| | 1H-inden-5-yl)-N-hydroxypropanamide |
| 63 | 3-(6-cyano-1-{(2-hydroxyethyl)[2-(1H-indol-3-yl)ethyl]amino}- |
| | 2,3dihydro-1H-inden-5-yl)-N-hydroxypropanamide |
| 64 | 6-[3-(hydroxyamino)-3-oxopropyl]-3-{(2-hydroxyethyl)[2-(1H-indol-3- |
| | yl)ethyl]amino}indane-5-carboxylic acid |
| 65 | 6-[(1E)-3-(hydroxyamino)-3-oxoprop-1-en-1-yl]-3-{(2-hydroxyethyl)[2-(1H- |
| | indol-3-yl)ethyl]amino}indane-5-carboxylic acid |
| 66 | (2E)-N-hydroxy-3-(1-{(2-hydroxyethyl)[2-(1H-indol-3-yl)ethyl]amino}-6- |
| | nitro-2,3-dihydro-1H-inden-5-yl)acrylamide |
| 67 | (2E)-3-(6-chloro-1-{(2-hydroxyethyl)[2-(1H-indol-3-yl)ethyl]amino}-2,3- |
| | dihydro-1H-inden-5-yl)-N-hydroxyacrylamide |
| 68 | (2E)-3-(6-cyano-1-{(2-hydroxyethyl)[2-(1H-indol-3-yl)ethyl]amino}-2,3- |
| | dihydro-1H-inden-5-yl)-N-hydroxyacrylamide |
| 69 | (2E)-3-(6-amino-1-{(2-hydroxyethyl)[2-(1H-indol-3-yl)ethyl]amino}-2,3- |
| | dihydro-1H-inden-5-yl)-N-hydroxyacrylamide |
| 70 | (2E)-3-(4,6-dichloro-1-{(2-hydroxyethyl)[2-(1H-indol-3-yl)ethyl]amino}-2,3- |
| | dihydro-1H-inden-5-yl)-N-hydroxyacrylamide |
| 71 | (2E)-3-(4-chloro-1-{(2-hydroxyethyl)[2-(1H-indol-3-yl)ethyl]amino}-2,3- |
| | dihydro-1H-inden-5-yl)-N-hydroxyacrylamide |
| 72 | (2E)-N-hydroxy-3-(1-{(1H-imidazol-4-ylmethyl)[2-(1H-indol-3- |
| | yl)ethyl]amino}-2,3-dihydro-1H-inden-5-yl)acrylamide |
| 73 | (2E)-3-(1-{(2,3-dihydroxypropyl)[2-(1H-indol-3-yl)ethyl]amino}-2,3-dihydro- |
| | 1H-inden-5-yl)-N-hydroxyacrylamide |
| 74 | (2E)-N-hydroxy-3-(1-{[2-(2-hydroxyethoxy)ethyl][2-(1H-indol-3- |
| | yl)ethyl]amino}-2,3-dihydro-1H-inden-5-yl)acrylamide |
| 75 | (2E)-3-(1-{[2-(2,3-dihydroxypropoxy)ethyl][2-(1H-indol-3-yl)ethyl]amino}- |
| | 2,3-dihydro-1H-inden-5-yl)-N-hydroxyacrylamide |
| 76 | (2E)-N-hydroxy-3-(1-{[2-(1H-indol-3-yl)ethyl][(2-(2-morpholin-4- |
| | ylethoxy)ethyl]amino}-2,3-dihydro-1H-inden-5-yl)acrylamide |
| 77 | (2E)-N-hydroxy-3-(1-{[2-(1H-imidazol-5-ylmethoxy)ethyl][2-(1H-indol-3- |
| | yl)ethyl]amino}-2,3-dihydro-1H-inden-5-yl)acrylamide |
| 78 | 2-{{5-[(1E)-3-(hydroxyamino)-3-oxoprop-1-en-1-yl]-2,3-dihydro-1H-inden- |
| | 1-yl}[2-(1H-indol-3-yl)ethyl]amino}ethyl diethylcarbamate |
| 79 | 2-{{5-[(1E)-3-(hydroxyamino)-3-oxoprop-1-en-1-yl]-2,3-dihydro-1H-inden- |
| | 1-yl}[2-(1H-indol-3-yl)ethyl]amino}ethyl pyrrolidine-1-carboxylate |
| 80 | 2-{{5-[(1E)-3-(hydroxyamino)-3-oxoprop-1-en-1-yl]-2,3-dihydro-1H-inden- |
| | 1-yl}[2-(1H-indol-3-yl)ethyl]amino}ethyl benzylcarbamate |
| 81 | (2E)-N-hydroxy-3-{1-[[2-(1H-indol-3-yl)ethyl](2-morpholin-4-ylethyl)amino]- |
| | 2,3-dihydro-1H-inden-5-yl}acrylamide |
| 82 | (2E)-3-(1-{[2-(diethylamino)ethyl][2-(1H-indol-3-yl)ethyl]amino}-2,3- |
| | dihydro-1H-inden-5-yl)-N-hydroxyacrylamide |
| 83 | (2E)-3-(1-{[2-(acetylamino)ethyl][2-(1H-indol-3-yl)ethyl]amino}-2,3-dihydro- |
| | 1H-inden-5-yl)-N-hydroxyacrylamide |
| 84 | (2E)-N-hydroxy-3-[1-([2-(1H-indol-3-yl)ethyl]{2- |
| | [(methylsulfonyl)amino]ethyl}amino)-2,3-dihydro-1H-inden-5-yl]acrylamide |
| 85 | (2E)-N-hydroxy-3-(1-{{2-[(2-hydroxyethyl)amino]ethyl}[2-(1H-indol-3- |
| | yl)ethyl]amino}-2,3-dihydro-1H-inden-5-yl)acrylamide |
| 86 | N-(2-{{5-[(1E)-3-(hydroxyamino)-3-oxoprop-1-en-1-yl]-2,3-dihydro-1H- |
| | inden-1-yl}[2-(1H-indol-3-yl)ethyl]amino}ethyl)-beta-D- |
| | glucopyranosylamine |
| 87 | (2E)-3-(1-{[2-(beta-D-glucopyranosyloxy)ethyl][2-(1H-indol-3- |
| | yl)ethyl]amino}-2,3-dihydro-1H-inden-5-yl)-N-hydroxyacrylamide |
| 88 | (2E)-N-hydroxy-3-(1-{[2-(1H-indol-3-yl)ethyl][2-(4-methylpiperazin-1- |
| | yl)ethyl]amino}-2,3-dihydro-1H-inden-5-yl)acrylamide |
| 89 | (2E)-3-(1-{[2-(4-acetylpiperazin-1-yl)ethyl][2-(1H-indol-3-yl)ethyl]amino}- |
| | 2,3-dihydro-1H-inden-5-yl)-N-hydroxyacrylamide |
| 90 | (2E)-N-hydroxy-3-[1-([2-(1H-indol-3-yl)ethyl]{2-[4- |
| | (methylsulfonyl)piperazin-1-yl]ethyl}amino)-2,3-dihydro-1H-inden-5- |
| | yl]acrylamide |
| 91 | (2E)-N-hydroxy-3-{1-[[2-(1H-indol-3-yl)ethyl](3-morpholin-4-yl-3- |
| | oxopropyl)amino]-2,3-dihydro-1H-inden-5-yl}acrylamide |
| 92 | N,N-diethyl-N3-{5-[(1E)-3-(hydroxyamino)-3-oxoprop-1-en-1-yl]-2,3- |
| | dihydro-1H-inden-1-yl}-N3-[2-(1H-indol-3-yl)ethyl]-beta-alaninamide |
| 93 | (2E)-3-(1-{ethyl[2-(1H-indol-3-yl)ethyl]amino}-2,3-dihydro-1H-inden-5-yl)- |
| | N-hydroxyacrylamide |
| 94 | (2E)-3-(1-{benzyl[2-(1H-indol-3-yl)ethyl]amino}-2,3-dihydro-1H-inden-5-yl)- |
| | N-hydroxyacrylamide |
| 95 | (2E)-3-(1-{(cyclohexylmethyl)[2-(1H-indol-3-yl)ethyl]amino}-2,3-dihydro- |
| | 1H-inden-5-yl)-N-hydroxyacrylamide |
| 96 | (2E)-3-(1-(cyclohexyl[2-(1H-indol-3-yl)ethyl]amino}-2,3-dihydro-1H-inden- |
| | 5-yl)-N-hydroxyacrylamide |
| 97 | (2E)-N-hydroxy-3-{1-[[2-(1H-indol-3-yl)ethyl](pyridin-2-ylmethyl)amino]-2,3- |
| | dihydro-1H-inden-5-yl}acrylamide |
| 98 | (2E)-N-hydroxy-3-{1-[[2-(1H-indol-3-yl)ethyl](phenyl)amino]-2,3-dihydro- |
| | 1H-inden-5-yl}acrylamide |
| 99 | (2E)-N-hydroxy-3-{1-[[2-(1H-indol-3-yl)ethyl](pyridin-2-yl)amino]-2,3- |
| | dihydro-1H-inden-5-yl}acrylamide |
| 100 | (2E)-N-hydroxy-3-{1-[[2-(1H-indol-3-yl)ethyl](tetrahydro-2H-pyran-4- |
| | yl)amino]-2,3-dihydro-1H-inden-5-yl}acrylamide |
| 101 | (2E)-N-hydroxy-3-(1-{[2-hydroxy-1-(hydroxymethyl)-2-phenylethyl]amino}- |
| | 2,3-dihydro-1H-inden-5-yl)acrylamide |
| 102 | (2E)-N-hydroxy-3-(1-{[2-hydroxy-1-(hydroxymethyl)-2-(4- |
| | nitrophenyl)ethyl]amino}-2,3-dihydro-1H-inden-5-yl)acrylamide |
| 103 | (2E)-N-hydroxy-3-[1-({2-hydroxy-1-(hydroxymethyl)-2-[4- |
| | (methylsulfonyl)phenyl]ethyl}amino)-2,3-dihydro-1H-inden-5-yl]acrylamide |
| 104 | (2E)-3-(1-{[1-(4-chlorobenzyl)-2-hydroxyethyl]amino}-2,3-dihydro-1H- |
| | inden-5-yl)-N-hydroxyacrylamide |
| 105 | (2E)-N-hydroxy-3-(1-{[2-hydroxy-1-(4-hydroxybenzyl)ethyl]amino}-2,3- |
| | dihydro-1H-inden-5-yl)acrylamide |
| 106 | (2E)-N-hydroxy-3-(1-{[2-hydroxy-1-(1H-imidazol-4-ylmethyl)ethyl]amino}- |
| | 2,3-dihydro-1H-inden-5-yl)acrylamide |
| 107 | (2E)-N-hydroxy-3-(1-{(2-hydroxy-1-(4-methoxybenzyl)ethyl]amino}-2,3- |
| | dihydro-1H-inden-5-yl)acrylamide |
| 108 | (2E)-N-hydroxy-3-(1-{[3-hydroxy-1-(hydroxymethyl)-3-(4- |
| | nitrophenyl)propyl]amino}-2,3-dihydro-1H-inden-5-yl)acrylamide |
| 109 | (2E)-N-hydroxy-3-{1-[(2-hydroxy-1-phenylethyl)amino]-2,3-dihydro-1H- |
| | inden-5-yl}acrylamide |
| 110 | (2E)-N-hydroxy-3-(1-{[3-hydroxy-1-(5-methyl-2-thienyl)propyl]amino}-2,3- |
| | dihydro-1H-inden-5-yl)acrylamide |
| 111 | (2E)-N-hydroxy-3-{1-[(3-hydroxy-1-pyridin-3-ylpropyl)amino]-2,3-dihydro- |
| | 1H-inden-5-yl}acrylamide |
| 112 | (2E)-3-[1-({1-[4-(dimethylamino)phenyl]-3-hydroxypropyl}amino)-2,3- |
| | dihydro-1H-inden-5-yl]-N-hydroxyacrylamide |
| 113 | (2E)-3-(1-{[1-(4-fluorophenyl)-3-hydroxypropyl]amino}-2,3-dihydro-1H- |
| | inden-5-yl)-N-hydroxyacrylamide |
| 114 | (2E)-3-(1-{[1-(3,4-dimethoxyphenyl)-3-hydroxypropyl]amino}-2,3-dihydro- |
| | 1H-inden-5-yl)-N-hydroxyacrylamide |
| 115 | (2E)-3-(1-{[1-(1-benzofuran-2-yl)-3-hydroxypropyl]amino}-2,3-dihydro-1H- |
| | inden-5-yl)-N-hydroxyacrylamide |
| 116 | (2E)-3-(1-{[1-(1-benzothien-3-yl)-3-hydroxypropyl]amino}-2,3-dihydro-1H- |
| | inden-5-yl)-N-hydroxyacrylamide |
| 117 | (2E)-N-hydroxy-3-{1-[(3-hydroxy-1-quinolin-3-ylpropyl)amino]-2,3-dihydro- |
| | 1H-inden-5-yl}acrylamide |
| 118 | (2E)-N-hydroxy-3-{1-[(2-hydroxy-1-pyridin-3-ylethyl)amino]-2,3-dihydro- |
| | 1H-inden-5-yl}acrylamide |
| 119 | tert-butyl N-{5-[(1E)-3-(hydroxyamino)-3-oxoprop-1-en-1-yl]-2,3-dihydro- |
| | 1H-inden-1-yl}tyrosinate |
| 120 | N-{5-[(1E)-3-(hydroxyamino)-3-oxoprop-1-en-1-yl]-2,3-dihydro-1H-inden-1- |
| | yl}tyrosine |
| 121 | tert-butyl N-{5-[(1E)-3-(hydroxyamino)-3-oxoprop-1-en-1-yl]-2,3-dihydro- |
| | 1H-inden-1-yl}tryptophanate |
| 122 | N-{5-[(1E)-3-(hydroxyamino)-3-oxoprop-1-en-1-yl]-2,3-dihydro-1H-inden-1- |
| | yl}trytophan |
| 123 | Nalpha-{5-[(1E)-3-(hydroxyamino)-3-oxoprop-1-en-1-yl]-2,3-dihydro-1H- |
| | inden-1-yl}-N-(2-hydroxyethyl)tryptophanamide |
| 124 | (2E)-N-hydroxy-3-(1-{[1-(1H-indol-3-ylmethyl)-2-morpholin-4-yl-2- |
| | oxoethyl]amino}-2,3-dihydro-1H-inden-5-yl)acrylamide |
| 125 | N-[2-(dimethylamino)ethyl]-Nalpha-{5-[(1E)-3-(hydroxyamino)-3-oxoprop- |
| | 1-en-1-yl]-2,3-dihydro-1H-inden-1-yl}tryptophanamide |
| 126 | Nalpha-{5-[(1E)-3-(hydroxyamino)-3-oxoprop-1-en-1-yl]-2,3-dihydro-1H- |
| | inden-1-yl}tryptophanamide |
| 127 | N-{5-[(1E)-3-(hydroxyamino)-3-oxoprop-1-en-1-yl]-2,3-dihydro-1H-inden-1- |
| | yl}tryptophylglycine |
| 128 | (2E)-N-hydroxy-3-(1-{[(1S)-1-(hydroxymethyl)-3-methylbutyl]amino}-2,3- |
| | dihydro-1H-inden-5-yl)acrylamide |
| 129 | (2E)-N-hydroxy-3-(1-{[1-(hydroxymethyl)-2-methylpropyl]amino}-2,3- |
| | dihydro-1H-inden-5-yl)acrylamide |
| 130 | (2E)-N-hydroxy-3-{1-[(2-hydroxyethyl)amino]-2,3-dihydro-1H-inden-5- |
| | yl}acrylamide |
| 131 | tert-butyl N-{5-[(1E)-3-(hydroxyamino)-3-oxoprop-1-en-1-yl]-2,3-dihydro- |
| | 1H-inden-1-yl}serinate |
| 132 | (2E)-N-hydroxy-3-(1-{[2-hydroxy-1-(hydroxymethyl)ethyl]amino}-2,3- |
| | dihydro-1H-inden-5-yl)acrylamide |
| 133 | N-{5-[(1E)-3-(hydroxyamino)-3-oxoprop-1-en-1-yl]-2,3-dihydro-1H-inden-1- |
| | yl}serine |
| 134 | (3-{2-[{5-[(1E)-3-(hydroxyamino)-3-oxoprop-1-en-1 -yl]-2,3-dihydro-1H- |
| | inden-1-yl}(2-hydroxyethyl)amino]ethyl}-1H-indol-1-yl)acetic acid |
| 135 | (2E)-N-hydroxy-3-[1-((2-hydroxyethyl){2-[1-(2-oxo-2-pyrrolidin-1-ylethyl)- |
| | 1H-indol-3-yl]ethyl}amino)-2,3-dihydro-1H-inden-5-yl]acrylamide |
| 136 | (2E)-3-{1-[(2-{1-[2-(diethylamino)-2-oxoethyl]-1H-indol-3-yl}ethyl)(2- |
| | hydroxyethyl)amino]-2,3-dihydro-1H-inden-5-yl}-N-hydroxyacrylamide |
| 137 | (2E)-3-{1-[{2-[1-(2-amino-2-oxoethyl)-1H-indol-3-yl]ethyl}(2- |
| | hydroxyethyl)amino]-2,3-dihydro-1H-inden-5-yl}-N-hydroxyacrylamide |
| 138 | (2E)-3-{1-[{2-[1-(2,3-dihydroxypropyl)-1H-indol-3-yl]ethyl}(2- |
| | hydroxyethyl)amino]-2,3-dihydro-1H-inden-5-yl}-N-hydroxyacrylamide |
| 139 | (2E)-N-hydroxy-3-[1-((2-hydroxyethyl){2-[1-(2-morpholin-4-ylethyl)-1H- |
| | indol-3-yl]ethyl}amino)-2,3-dihydro-1H-inden- |
| | 5-yl]acrylamide |
| 140 | (2E)-N-hydroxy-3-{1-[(2-hydroxyethyl)(2-{1-[2-(1H-imidazol-5-yl)ethyl]-1H- |
| | indol-3-yl}ethyl)amino]-2,3-dihydro-1H-inden-5-yl}acrylamide |
| 141 | (2E)-N-hydroxy-3-(1-{(2-hydroxyethyl)[2-(5-methyl-1H-indol-3- |
| | yl)ethyl]amino}-2,3-dihydro-1H-inden-5-yl)acrylamide |
| 142 | (2E)-N-hydroxy-3-(1-{(2-hydroxyethyl)[2-(5-methoxy-1H-indol-3- |
| | yl)ethyl]amino}-2,3-dihydro-1H-inden-5-yl)acrylamide |
| 143 | (2E)-3-{1-[[2-(5-chloro-1H-indol-3-yl)ethyl](2-hydroxyethyl)amino]-2,3- |
| | dihydro-1H-inden-5-yl}-N-hydroxyacrylamide |
| 144 | (2E)-3-{1-[[2-(5-fluoro-2-methyl-1H-indol-3-yl)ethyl](2-hydroxyethyl)amino]- |
| | 2,3-dihydro-1H-inden-5-yl}-N-hydroxyacrylamide |
| 145 | (2E)-N-hydroxy-3-(6-{(2-hydroxyethyl)[2-(1H-indol-3-yl)ethyl]amino}- |
| | 5,6,7,8-tetrahydronaphthalen-2-yl)acrylamide |
| 146 | (2E)-N-hydroxy-3-(6-{[2-(1H-indol-3-yl)ethyl]amino)-5,6,7,8- |
| | tetrahydronaphthalen-2-yl)acrylamide |
| 147 | (2E)-N-hydroxy-3-(1-{(2-hydroxyethyl)[2-(1H-indol-3-yl)ethyl]amino}-6,7- |
| | dimethyl-2,3-dihydro-1H-inden-5-yl)acrylamide |
| 148 | (2E)-3-(6,7-dichloro-1-{(2-hydroxyethyl)[2-(1H-indol-3-yl)ethyl]amino}-2,3- |
| | dihydro-1H-inden-5-yl)-N-hydroxyacrylamide |
| 149 | (2E)-N-hydroxy-3-(1-{(2-hydroxyethyl)[2-(1H-indol-3-yl)ethyl]amino}-7- |
| | methyl-2,3-dihydro-1H-inden-5-yl)acrylamide |
| 150 | (2E)-N-hydroxy-3-(1-{(2-hydroxyethyl)[2-(2-thienyl)ethyl]amino}-2,3- |
| | dihydro-1H-inden-5-yl)acrylamide |
| 151 | (2E)-N-hydroxy-3-[1-((2-hydroxyethyl){2-[3- |
| | (trifluoromethyl)phenyl]ethyl}amino)-2,3-dihydro-1H-inden-5-yl]acrylamide |
| 152 | (2E)-N-hydroxy-3-{1-[(2-hydroxyethyl)(2-{4- |
| | [(methylsulfonyl)amino]phenyl}ethyl)amino]-2,3-dihydro-1H-inden-5- |
| | yl}acrylamide |
| 153 | (2E)-3-{1-[(4-cyanobenzyl)(2-hydroxyethyl)amino]-2,3-dihydro-1H-inden-5- |
| | yl)-N-hydroxyacrylamide |
| 154 | (2E)-3-{1-[[4-(ethylamino)benzyl](2-hydroxyethyl)amino]-2,3-dihydro-1H- |
| | inden-5-yl}-N-hydroxyacrylamide |
| 155 | N-ethyl-4-{[{5-[(1E)-3-(hydroxyamino)-3-oxoprop-1-en-1-yl]-2,3-dihydro- |
| | 1H-inden-1-yl}(2-hydroxyethyl)amino]methyl}benzamide |
| 156 | 4-{[{5-[(1E)-3-(hydroxyamino)-3-oxoprop-1-en-1-yl]-2,3-dihydro-1H-inden- |
| | 1-yl}(2-hydroxyethyl)amino]methyl}benzoic acid |
| 157 | (2E)-N-hydroxy-3-[1-((2-hydroxyethyl){2-[1-(2-methoxyethyl)-1H-indol-3- |
| | yl]ethyl}amino)-2,3-dihydro-1H-inden-5-yl]acrylamide |
| 158 | (2E)-3-{1-[(2-{1-[2-(dimethylamino)ethyl]-1H-indol-3-yl}ethyl)(2- |
| | hydroxyethyl)amino]-2,3-dihydro-1H-inden-5-yl}-N-hydroxyacrylamide |
| 159 | (2E)-N-hydroxy-3-[1-((2-hydroxyethyl){2-[1-(2-pyrrolidin-1-ylethyl)-1H- |
| | indol-3-yl]ethyl}amino)-2,3-dihydro-1H-inden-5-yl]acrylamide |
| 160 | (2E)-N-hydroxy-3-[1-((2-hydroxyethyl){2-[5-(2-methoxyethoxy)-1H-indol-3- |
| | yl]ethyl}amino)-2,3-dihydro-1H-inden-5-yl]acrylamide |
| 161 | (2E)-3-{1-[(2-{5-[2-(dimethylamino)ethoxy]-1H-indol-3-yl}ethyl)(2- |
| | hydroxyethyl)amino]-2,3-dihydro-1H-inden-5-yl}-N-hydroxyacrylamide |
| 162 | (2E)-N-hydroxy-3-[1-((2-hydroxyethyl){2-[5-(2-morpholin-4-ylethoxy)-1H- |
| | indol-3-yl]ethyl}amino)-2,3-dihydro-1H-inden-5-yl]acrylamide |
| 163 | (2E)-N-hydroxy-3-[1-((2-hydroxyethyl){2-[5-(2-pyrrolidin-1-ylethoxy)-1H- |
| | indol-3-yl]ethyl}amino)-2,3-dihydro-1H-inden-5-yl]acrylamide |
| 164 | (2E)-N-hydroxy-3-{1-[(2-hydroxyethyl)(2-{5-[2-(1H-imidazol-5-yl)ethoxy]- |
| | 1H-indol-3-yl}ethyl)amino]-2,3-dihydro-1H-inden-5-yl}acrylamide |
| 165 | (2E)-3-(1-{(2-{[(ethylamino)carbonyl]amino}ethyl)[2-(1H-indol-3- |
| | yl)ethyl]amino}-2,3-dihydro-1H-inden-5-yl)-N-hydroxyacrylamide |
| 166 | (2E)-N-hydroxy-3-[1-((2-hydroxyethyl){2-[1-(3-morpholin-4-yl-3-oxopropyl)- |
| | 1H-indol-3-yl]ethyl}amino)-2,3-dihydro-1H-inden-5-yl]acrylamide |
| 167 | N-ethyl-Nalpha-{5-[(1E)-3-(hydroxyamino)-3-oxoprop-1-en-1-yl]-2,3- |
| | dihydro-1H-inden-1-yl}tryptophanamide |
| 168 | Nalpha-{5-[(1E)-3-(hydroxyamino)-3-oxoprop-1-en-1-yl]-2,3-dihydro-1H- |
| | inden-1-yl}-N,N-dimethyltryptophanamide |
| 169 | (2E)-3-(1-{{[(4-fluorophenyl)amino]carbonyl}[2-(1H-indol-3-yl)ethyl]amino}- |
| | 2,3-dihydro-1H-inden-5-yl)-N-hydroxyacrylamide |
| 170 | 2-{{5-[(1E)-3-(hydroxyamino)-3-oxoprop-1-en-1-yl]-2,3-dihydro-1H-inden- |
| | 1-yl}[2-(1H-indol-3-yl)ethyl]amino}ethyl ethylcarbamate |
| 171 | 2-{{5-[(1E)-3-(hydroxyamino)-3-oxoprop-1-en-1-yl]-2,3-dihydro-1H-inden- |
| | 1-yl}[2-(1H-indol-3-yl)ethyl]amino}ethyl phenylcarbamate |
| 172 | (2E)-N-hydroxy-3-(1-{[2-(1H-indol-3-yl)-1-methylethyl]amino}-2,3-dihydro- |
| | 1H-inden-5-yl)acrylamide |
| 173 | (2E)-3-{(1S)-1-[[2-(5-chloro-1H-indol-3-yl)ethyl](2-hydroxyethyl)amino]-2,3- |
| | dihydro-1H-inden-5-yl}-N-hydroxyacrylamide |
| 174 | (2E)-3-{(1R)-1-[[2-(5-chloro-1H-indol-3-yl)ethyl](2-hydroxyethyl)amino]-2,3- |
| | dihydro-1H-inden-5-yl}-N-hydroxyacrylamide |
| 175 | (2E)-3-(1-{ethyl[2-(3-methylphenyl)ethyl]amino}-4-fluoro-2,3-dihydro-1H- |
| | inden-5-yl)-N-hydroxyacrylamide |
| 176 | (2E)-3-(4-fluoro-1-{[2-(1H-indol-3-yl)ethyl]amino}-2,3-dihydro-1H-inden-5- |
| | yl)-N-hydroxyacrylamide |
| 177 | (2E)-3-{(1S)-1-[[2-(2-chlorophenyl)ethyl](3-hydroxypropyl)amino]-2,3- |
| | dihydro-1H-inden-5-yl}-N-hydroxyacrylamide |
| 178 | (2E)-3-{(1R)-1-[[2-(2-chlorophenyl)ethyl](3-hydroxypropyl)amino]-2,3- |
| | dihydro-1H-inden-5-yl}-N-hydroxyacrylamide |
| 179 | (2E)-N-hydroxy-3-(1-{methyl[2-(1-methyl-1H-indol-3-yl)ethyl]amino}-2,3- |
| | dihydro-1H-inden-5-yl)acrylamide |
| 180 | (2E)-N-hydroxy-3-(1-{(2-hydroxyethyl)[2-hydroxy-1-(1H-indol-3- |
| | ylmethyl)ethyl]amino}-2,3-dihydro-1H-inden-5-yl)acrylamide |
| 181 | N-{(1R)-5-[(1E)-3-(hydroxyamino)-3-oxoprop-1-en-1-yl]-2,3-dihydro-1H- |
| | inden-1-yl}-N-[(1S)-2-hydroxy-1-(1H-indol-3- |
| | ylmethyl)ethyl]cyclobutanecarboxamide |
| 182 | N-{(1R)-5-[(1E)-3-(hydroxyamino)-3-oxoprop-1-en-1-yl]-2,3-dihydro-1H- |
| | inden-1-yl}-N-[(1S)-2-hydroxy-1-(1H-indol-3-ylmethyl)ethyl]benzamide |
| 183 | N-{(1R)-5-[(1E)-3-(hydroxyamino)-3-oxoprop-1-en-1-yl]-2,3-dihydro-1H- |
| | inden-1-yl}-N-[(1S)-2-hydroxy-1-(1H-indol-3- |
| | ylmethyl)ethyl]cyclohexanecarboxamide |
| 184 | (2E)-3-((1S)-1-{[2-(5-chloro-1H-indol-3-yl)ethyl]amino}-2,3-dihydro-1H- |
| | inden-5-yl)-N-hydroxyacrylamide |
| 185 | (2E)-3-((1R)-1-{[2-(5-chloro-1H-indol-3-yl)ethyl]amino}-2,3-dihydro-1H- |
| | inden-5-yl)-N-hydroxyacrylamide |
| 186 | (2E)-3-(1-{ethyl[2-(1H-indol-3-yl)ethyl]amino}-2,3-dihydro-1H-inden-5-yl)- |
| | N-hydroxybut-2-enamide |
| 187 | (2E)-N-hydroxy-3-(1-{(3-hydroxypropyl)[2-(1H-indol-3-yl)ethyl]amino}-2,3- |
| | dihydro-1H-inden-5-yl)but-2-enamide |
| 188 | (2E)-3-(1-{[2-(5-fluoro-2-methyl-1H-indol-3-yl)ethyl]amino}-2,3-dihydro-1H- |
| | inden-5-yl)-N-hydroxybut-2-enamide |
| 189 | (2E)-3-(1-{[2-(5-chloro-1H-indol-3-yl)ethyl]amino}-2,3-dihydro-1H-inden-5- |
| | yl)-N-hydroxybut-2-enamide |
| 190 | (2E)-N-hydroxy-3-(1-{[2-(5-methoxy-1H-indol-3-yl)ethyl]amino}-2,3- |
| | dihydro-1H-inden-5-yl)but-2-enamide |
| 191 | (2E)-N-hydroxy-3-(1-{[2-(6-methoxy-1H-indol-3-yl)ethyl]amino}-2,3- |
| | dihydro-1H-inden-5-yl)but-2-enamide |
| 192 | (2E)-3-(1-{[2-(6-fluoro-1H-indol-3-yl)ethyl]amino}-2,3-dihydro-1H-inden-5- |
| | yl)-N-hydroxybut-2-enamide |
| 193 | (2E)-N-hydroxy-3-((1S)-1-{(2-hydroxyethyl)[2-(1H-indol-3-yl)ethyl]amino}- |
| | 2,3-dihydro-1H-inden-5-yl)but-2-enamide |
| 194 | (2E)-N-hydroxy-3-((1R)-1-{(2-hydroxyethyl)[2-(1H-indol-3-yl)ethyl]amino}- |
| | 2,3-dihydro-1H-inden-5-yl)but-2-enamide |
| 195 | (2E)-3-(1-{ethyl[2-(6-methoxy-1H-indol-3-yl)ethyl]amino}-2,3-dihydro-1H- |
| | inden-5-yl)-N-hydroxybut-2-enamide |
| 196 | (2E)-3-(1-{ethyl[2-(5-methoxy-1H-indol-3-yl)ethyl]amino}-2,3-dihydro-1H- |
| | inden-5-yl)-N-hydroxybut-2-enamide |
| 197 | (2E)-3-(1-{ethyl[2-(6-fluoro-1H-indol-3-yl)ethyl]amino}-2,3-dihydro-1H- |
| | inden-5-yl)-N-hydroxybut-2-enamide |
| 198 | (2E)-N-hydroxy-3-(1-{[2-(6-methoxy-1H-indol-3-yl)ethyl]amino}-2,3- |
| | dihydro-1H-inden-5-yl)acrylamide |
| 199 | (2E)-N-hydroxy-3-(1-{[2-(5-methoxy-1H-indol-3-yl)ethyl]amino}-2,3- |
| | dihydro-1H-inden-5-yl)acrylamide |
| 200 | (2E)-N-hydroxy-3-(1-{(2-hydroxyethyl)[2-(6-methoxy-1H-indol-3- |
| | yl)ethyl]amino}-2,3-dihydro-1H-inden-5-yl)acrylamide |
| 201 | (2E)-3-(1-{[2-(5-fluoro-2-methyl-1H-indol-3-yl)ethyl]amino}-2,3-dihydro-1H- |
| | inden-5-yl)-N-hydroxyacrylamide |
| 202 | (2E)-3-(1-{[2-(5-chloro-1H-indol-3-yl)ethyl]amino}-2,3-dihydro-1H-inden-5- |
| | yl)-N-hydroxyacrylamide |
| 203 | (2E)-N-hydroxy-3-{1-[(2-phenylethyl)amino]-2,3-dihydro-1H-inden-5- |
| | yl}acrylamide |
| 204 | (2E)-N-hydroxy-3-(1-{[2-(2-methylphenyl)ethyl]amino}-2,3-dihydro-1H- |
| | inden-5-yl)acrylamide |
| 205 | (2E)-N-hydroxy-3-(1-{[2-(4-methylphenyl)ethyl]amino}-2,3-dihydro-1H- |
| | inden-5-yl)acrylamide |
| 206 | (2E)-N-hydroxy-3-(1-{[2-(3-methylphenyl)ethyl]amino}-2,3-dihydro-1H- |
| | inden-5-yl)acrylamide |
| 207 | (2E)-N-hydroxy-3-((1S)-1-{(3-hydroxypropyl)[2-(1-methyl-1H-indol-3- |
| | yl)ethyl]amino}-2,3-dihydro-1H-inden-5-yl)acrylamide |
| 208 | (2E)-N-hydroxy-3-(1-{(2-hydroxyethyl)[2-(2-methylphenyl)ethyl]amino}-2,3- |
| | dihydro-1H-inden-5-yl)acrylamide |
| 209 | (2E)-N-hydroxy-3-(1-{(2-hydroxyethyl)[2-(4-methylphenyl)ethyl]amino}-2,3- |
| | dihydro-1H-inden-5-yl)acrylamide |
| 210 | (2E)-N-hydroxy-3-(1-{(2-hydroxyethyl)[2-(3-methylphenyl)ethyl]amino}-2,3- |
| | dihydro-1H-inden-5-yl)acrylamide |
| 211 | (2E)-N-hydroxy-3-{1-[(2-hydroxyethyl)(2-phenylethyl)amino]-2,3-dihydro- |
| | 1H-inden-5-yl}acrylamide |
| 212 | (2E)-N-hydroxy-3-((1R)-1-{(3-hydroxypropyl)[2-(1-methyl-1H-indol-3- |
| | yl)ethyl]amino}-2,3-dihydro-1H-inden-5-yl)acrylamide |
| 213 | (2E)-3-(1-{[2-(2-chlorophenyl)ethyl]amino}-2,3-dihydro-1H-inden-5-yl)-N- |
| | hydroxyacrylamide |
| 214 | (2E)-N-hydroxy-3-(1-{[2-(2-methoxyphenyl)ethyl]amino}-2,3-dihydro-1H- |
| | inden-5-yl)acrylamide |
| 215 | (2E)-3-(1-{[2-(2,3-dimethoxyphenyl)ethyl]amino}-2,3-dihydro-1H-inden-5- |
| | yl)-N-hydroxyacrylamide |
| 216 | (2E)-3-(1-{[2-(3-chlorophenyl)ethyl]amino}-2,3-dihydro-1H-inden-5-yl)-N- |
| | hydroxyacrylamide |
| 217 | (2E)-3-(1-{[2-(3,4-dimethylphenyl)ethyl]amino}-2,3-dihydro-1H-inden-5-yl)- |
| | N-hydroxyacrylamide |
| 218 | (2E)-N-hydroxy-3-(1-{[2-(3-methoxyphenyl)ethyl]amino}-2,3-dihydro-1H- |
| | inden-5-yl)acrylamide |
| 219 | (2E)-N-hydroxy-3-(1-{[2-(4-methoxyphenyl)ethyl]amino}-2,3-dihydro-1H- |
| | inden-5-yl)acrylamide |
| 220 | (2E)-3-(1-{[2-(4-chlorophenyl)ethyl]amino}-2,3-dihydro-1H-inden-5-yl)-N- |
| | hydroxyacrylamide |
| 221 | (2E)-3-(1-{[2-(3-bromo-4-methoxyphenyl)ethyl]amino}-2,3-dihydro-1H- |
| | inden-5-yl)-N-hydroxyacrylamide |
| 222 | (2E)-3-{1-[[2-(2-chlorophenyl)ethyl](3-hydroxypropyl)amino]-2,3-dihydro- |
| | 1H-inden-5-yl}-N-hydroxyacrylamide |
| 223 | (2E)-3-{1-[[2-(2,3-dimethoxyphenyl)ethyl](3-hydroxypropyl)amino]-2,3- |
| | dihydro-1H-inden-5-yl}-N-hydroxyacrylamide |
| 224 | (2E)-N-hydroxy-3-(1-{(3-hydroxypropyl)[2-(3-methoxyphenyl)ethyl]amino}- |
| | 2,3-dihydro-1H-inden-5-yl)acrylamide |
| 225 | (2E)-N-hydroxy-3-(1-{(3-hydroxypropyl)[2-(2-methoxyphenyl)ethyl]amino}- |
| | 2,3-dihydro-1H-inden-5-yl)acrylamide |
| 226 | (2E)-N-hydroxy-3-(1-{(3-hydroxypropyl)[2-(4-methoxyphenyl)ethyl]amino}- |
| | 2,3-dihydro-1H-inden-5-yl)acrylamide |
| 227 | (2E)-3-{1-[[2-(3-chlorophenyl)ethyl](3-hydroxypropyl)amino]-2,3-dihydro- |
| | 1H-inden-5-yl)-N-hydroxyacrylamide |
| 228 | (2E)-3-{1-[[2-(4-chlorophenyl)ethyl](3-hydroxypropyl)amino]-2,3-dihydro- |
| | 1H-inden-5-yl}-N-hydroxyacrylamide |
| 229 | (2E)-N-hydroxy-3-((1S)-1-{(2-hydroxyethyl)[2-(2- |
| | methylphenyl)ethyl]amino}-2,3-dihydro-1H-inden-5-yl)acrylamide |
| 230 | (2E)-N-hydroxy-3-((1R)-1-{(2-hydroxyethyl)[2-(2- |
| | methylphenyl)ethyl]amino}-2,3-dihydro-1H-inden-5-yl)acrylamide |
| 231 | (2E)-3-{1-[[2-(2,5-dimethoxyphenyl)ethyl](2-hydroxyethyl)amino]-2,3- |
| | dihydro-1H-inden-5-yl}-N-hydroxyacrylamide |
| 232 | (2E)-3-{1-[[2-(2,5-dimethylphenyl)ethyl](2-hydroxyethyl)amino]-2,3- |
| | dihydro-1H-inden-5-yl}-N-hydroxyacrylamide |
| 233 | (2E)-3-{1-[[2-(3,5-dimethoxyphenyl)ethyl](2-hydroxyethyl)amino]-2,3- |
| | dihydro-1H-inden-5-yl}-N-hydroxyacrylamide |
| 234 | (2E)-3-{1-[[2-(2,4-dimethylphenyl)ethyl](2-hydroxyethyl)amino]-2,3- |
| | dihydro-1H-inden-5-yl}-N-hydroxyacrylamide |
| 235 | (2E)-3-{1-[[2-(2,6-dichlorophenyl)ethyl](2-hydroxyethyl)amino]-2,3-dihydro- |
| | 1H-inden-5-yl}-N-hydroxyacrylamide |
| 236 | (2E)-3-{1-[[2-(2-fluorophenyl)ethyl](2-hydroxyethyl)amino]-2,3-dihydro-1H- |
| | inden-5-yl}-N-hydroxyacrylamide |
| 237 | (2E)-3-{1-[[2-(3-fluorophenyl)ethyl](2-hydroxyethyl)amino]-2,3-dihydro-1H- |
| | inden-5-yl}-N-hydroxyacrylamide |
| 238 | (2E)-3-{1-[[2-(4-fluorophenyl)ethyl](2-hydroxyethyl)amino]-2,3-dihydro-1H- |
| | inden-5-yl}-N-hydroxyacrylamide |
| 239 | (2E)-N-hydroxy-3-((1R)-1-{(4-hydroxybenzyl)[2-(1H-indol-3- |
| | yl)ethyl]amino}-2,3-dihydro-1H-inden-5-yl)acrylamide |
| 240 | (2E)-N-hydroxy-3-((1R)-1-{(4-hydroxybenzyl)[2-(1H-indol-3- |
| | yl)ethyl]amino}-2,3-dihydro-1H-inden-5-yl)acrylamide |
| 241 | (2E)-N-hydroxy-3-((1S)-1-{(4-hydroxybenzyl)[2-(1H-indol-3- |
| | yl)ethyl]amino}-2,3-dihydro-1H-inden-5-yl)acrylamide |
| 242 | (2E)-N-hydroxy-3-{1-[(2-hydroxyethyl)(3-phenylpropyl)amino]-2,3-dihydro- |
| | 1H-inden-5-yl}acrylamide |
| 243 | (2E)-N-hydroxy-3-(1-{(2-hydroxyethyl)[2-(2-methylphenoxy)ethyl]amino}- |
| | 2,3-dihydro-1H-inden-5-yl)acrylamide |
| 244 | (2E)-N-hydroxy-3-(1-{(2-hydroxyethyl)[2-(2-methoxyphenoxy)ethyl]amino}- |
| | 2,3-dihydro-1H-inden-5-yl)acrylamide |
| 245 | (2E)-N-hydroxy-3-(1-{(2-hydroxyethyl)[2-(4-methoxyphenoxy)ethyl]amino}- |
| | 2,3-dihydro-1H-inden-5-yl)acrylamide |
| 246 | (2E)-3-{1-[[2-(2-fluorophenoxy)ethyl](2-hydroxyethyl)amino]-2,3-dihydro- |
| | 1H-inden-5-yl}-N-hydroxyacrylamide |
| 247 | (2E)-N-hydroxy-3-{1-[(2-hydroxyethyl)(2-phenoxyethyl)amino]-2,3-dihydro- |
| | 1H-inden-5-yl}acrylamide |
| 248 | (2E)-3-{1-[[2-(2-chlorophenoxy)ethyl](2-hydroxyethyl)amino]-2,3-dihydro- |
| | 1H-inden-5-yl}-N-hydroxyacrylamide |
| 249 | (2E)-3-{1-[[2-(4-chlorophenoxy)ethyl](2-hydroxyethyl)amino]-2,3-dihydro- |
| | 1H-inden-5-yl}-N-hydroxyacrylamide |
| 250 | (2E)-N-hydroxy-3-(1-{(2-hydroxyethyl)[2-(3-methylphenoxy)ethyl]amino}- |
| | 2,3-dihydro-1H-inden-5-yl)acrylamide |
| 251 | (2E)-N-hydroxy-3-(1-{(2-hydroxyethyl)[2-(3-methoxyphenoxy)ethyl]amino}- |
| | 2,3-dihydro-1H-inden-5-yl)acrylamide |
| 252 | (2E)-3-{1-[[2-(3-fluorophenoxy)ethyl](2-hydroxyethyl)amino]-2,3-dihydro- |
| | 1H-inden-5-yl}-N-hydroxyacrylamide |
| 253 | (2E)-3-{1-[[2-(3-chlorophenoxy)ethyl](2-hydroxyethyl)amino]-2,3-dihydro- |
| | 1H-inden-5-yl}-N-hydroxyacrylamide |
| 254 | (2E)-N-hydroxy-3-((1S)-1-{(2-hydroxyethyl)[2-(6-methoxy-1H-indol-3- |
| | yl)ethyl]amino}-2,3-dihydro-1H-inden-5-yl)acrylamide |
| 255 | (2E)-N-hydroxy-3-((1R)-1-{[2-(6-methoxy-1H-indol-3-yl)ethyl]amino}-2,3- |
| | dihydro-1H-inden-5-yl)acrylamide |
| 256 | (2E)-N-hydroxy-3-((1S)-1-{[2-(6-methoxy-1H-indol-3-yl)ethyl]amino}-2,3- |
| | dihydro-1H-inden-5-yl)acrylamide |
| 257 | (2E)-N-hydroxy-3-((1R)-1-{(2-hydroxyethyl)[2-(6-methoxy-1H-indol-3- |
| | yl)ethyl]amino}-2,3-dihydro-1H-inden-5-yl)acrylamide |
| 258 | 5-({{5-[(1E)-3-(hydroxyamino)-3-oxoprop-1-en-1-yl]-2,3-dihydro-1H-inden- |
| | 1-yl}[2-(1H-indol-3-yl)ethyl]amino}methyl)-N,N-dimethyl-1H-pyrrole-3- |
| | carboxamide |
| 259 | (2E)-N-hydroxy-3-[1-([2-(1H-indol-3-yl)ethyl]{[4-(morpholin-4-ylcarbonyl)- |
| | 1H-pyrrol-2-yl]methyl}amino)-2,3-dihydro-1H-inden-5-yl]acrylamide |
| 260 | 5-({{5-[(1E)-3-(hydroxyamino)-3-oxoprop-1-en-1-yl]-2,3-dihydro-1H-inden- |
| | 1-yl}[2-(1H-indol-3-yl)ethyl]amino}methyl)-N-methyl-1H-pyrrole-3- |
| | carboxamide |
| 261 | (2E)-N-hydroxy-3-[1-([2-(1H-indol-3-yl)ethyl]{2- |
| | [(methylsulfonyl]amino]ethyl}amino)-2,3-dihydro-1H-inden-5-yl]acrylamide |
| 262 | (2E)-N-hydroxy-3-[1-([2-(1H-indol-3-yl)ethyl]{2- |
| | [(methylsulfonyl)amino]ethyl}amino)-2,3-dihydro-1H-inden-5-yl]acrylamide |
| 263 | 5-({{(1S)-5-[(1E)-3-(hydroxyamino)-3-oxoprop-1-en-1-yl]-2,3-dihydro-1H- |
| | inden-1-yl}[2-(1H-indol-3-yl)ethyl]amino}methyl)-N,N-dimethyl-1H-pyrrole- |
| | 3-carboxamide |
| 264 | 5-({{(1R)-5-[(1E)-3-(hydroxyamino)-3-oxoprop-1-en-1-yl]-2,3-dihydro-1H- |
| | inden-1-yl}[2-(1H-indol-3-yl)ethyl]amino}methyl)-N,N-dimethyl-1H-pyrrole- |
| | 3-carboxamide |
| 265 | tert-butyl {5-[(1E)-3-(hydroxyamino)-3-oxoprop-1-en-1-yl]-2,3-dihydro-1H- |
| | inden-1-yl}[2-(1H-indol-3-yl)ethyl]carbamate |
| 266 | (2E)-3-(1-{(2-fluorobenzyl)[2-(1H-indol-3-yl)ethyl]amino}-2,3-dihydro-1H- |
| | inden-5-yl)-N-hydroxyacrylamide |
| 267 | (2E)-3-(1-{butyl[2-(1H-indol-3-yl)ethyl]amino}-2,3-dihydro-1H-inden-5-yl)- |
| | N-hydroxyacrylamide |
| 268 | (2E)-N-hydroxy-3-(1-{(2-hydroxy-3-methoxybenzyl)[2-(1H-indol-3- |
| | yl)ethyl]amino}-2,3-dihydro-1H-inden-5-yl)acrylamide |
| 269 | (2E)-3-(1-{butyl[2-(1H-indol-3-yl)ethyl]amino}-2,3-dihydro-1H-inden-5-yl)- |
| | N-hydroxyacrylamide |
| 270 | (2E)-N-hydroxy-3-{1-[[2-(1H-indol-3-yl)ethyl](propyl)amino]-2,3-dihydro- |
| | 1H-inden-5-yl}acrylamide |
| 271 | (2E)-N-hydroxy-3-{1-[[2-(1H-indol-3-yl)ethyl](2-phenylethyl)amino]-2,3- |
| | dihydro-1H-inden-5-yl}acrylamide |
| 272 | (2E)-3-(1-{(2,5-dihydroxybenzyl)[2-(1H-indol-3-yl)ethyl]amino}-2,3-dihydro- |
| | 1H-inden-5-yl)-N-hydroxyacrylamide |
| 273 | (2E)-3-(1-{(5-chloro-2-hydroxybenzyl)[2-(1H-indol-3-yl)ethyl]amino}-2,3- |
| | dihydro-1H-inden-5-yl)-N-hydroxyacrylamide |
| 274 | (2E)-3-(1-{(2,3-dihydroxybenzyl)[2-(1H-indol-3-yl)ethyl]amino}-2,3-dihydro- |
| | 1H-inden-5-yl)-N-hydroxyacrylamide |
| 275 | (2E)-3-(1-{(cyclopentylmethyl)[2-(1H-indol-3-yl)ethyl]amino}-2,3-dihydro- |
| | 1H-inden-5-yl)-N-hydroxyacrylamide |
| 276 | (2E)-N-hydroxy-3-(1-{(2-hydroxy-3-methylbenzyl)[2-(1H-indol-3- |
| | yl)ethyl]amino)-2,3-dihydro-1H-inden-5-yl)acrylamide |
| 277 | (2E)-3-(1-{(5-fluoro-2-hydroxybenzyl)[2-(1H-indol-3-yl)ethyl]amino}-2,3- |
| | dihydro-1H-inden-5-yl)-N-hydroxyacrylamide |
| 278 | (2E)-N-hydroxy-3-{1-[[2-(1H-indol-3-yl)ethyl](isobutyl)amino]-2,3-dihydro- |
| | 1H-inden-5-yl}acrylamide |
| 279 | (2E)-3-(1-{(3,3-dimethylbutyl)[2-(1H-indol-3-yl)ethyl]amino}-2,3-dihydro- |
| | 1H-inden-5-yl)-N-hydroxyacrylamide |
| 280 | (2E)-N-hydroxy-3-{1-[[2-(1H-indol-3-yl)ethyl](1H-pyrrol-2-ylmethyl)amino]- |
| | 2,3-dihydro-1H-inden-5-yl}acrylamide |
| 281 | (2E)-3-(1-{(cyclopropylmethyl)[2-(1H-indol-3-yl)ethyl]amino}-2,3-dihydro- |
| | 1H-inden-5-yl)-N-hydroxyacrylamide |
| 282 | (2E)-N-hydroxy-3-{1-[[2-(1H-indol-3-yl)ethyl](pyridin-4-ylmethyl)amino]-2,3- |
| | dihydro-1H-inden-5-yl}acrylamide |
| 283 | (2E)-N-hydroxy-3-(1-{[2-(1H-indol-3-yl)ethyl][(1-methyl-1H-imidazol-2- |
| | yl)methyl]amino}-2,3-dihydro-1H-inden-5-yl)acrylamide |
| 284 | (2E)-N-hydroxy-3-{1-[[2-(1H-indol-3-yl)ethyl](2-thienylmethyl)amino]-2,3- |
| | dihydro-1H-inden-5-yl}acrylamide |
| 285 | (2E)-3-(1-{(2-furylmethyl)[2-(1H-indol-3-yl)ethyl]amino)-2,3-dihydro-1H- |
| | inden-5-yl)-N-hydroxyacrylamide |
| 286 | (2E)-N-hydroxy-3-{1-[[2-(1H-indol-3-yl)ethyl](2-phenoxyethyl)amino]-2,3- |
| | dihydro-1H-inden-5-yl}acrylamide |
| 287 | (2E)-3-(1-{[2-(2-chlorophenoxy)ethyl][2-(1H-indol-3-yl)ethyl]amino}-2,3- |
| | dihydro-1H-inden-5-yl)-N-hydroxyacrylamide |
| 288 | (2E)-3-(1-{[2-(3-chlorophenoxy)ethyl][2-(1H-indol-3-yl)ethyl]amino}-2,3- |
| | dihydro-1H-inden-5-yl)-N-hydroxyacrylamide |
| 289 | (2E)-3-(1-{[2-(4-chlorophenoxy)ethyl][2-(1H-indol-3-yl)ethyl]amino}-2,3- |
| | dihydro-1H-inden-5-yl)-N-hydroxyacrylamide |
| 290 | (2E)-N-hydroxy-3-(1-{[2-(1H-indol-3-yl)ethyl][(2-methyl-1H-imidazol-4- |
| | yl)methyl]amino}-2,3-dihydro-1H-inden-5-yl)acrylamide |
| 291 | (2E)-N-hydroxy-3-(1-{[2-(1H-indol-3-yl)ethyl][2-(2- |
| | methylphenoxy)ethyl]amino}-2,3-dihydro-1H-inden-5-yl)acrylamide |
| 292 | (2E)-N-hydroxy-3-(1-{[2-(1H-indol-3-yl)ethyl][2-(3- |
| | methylphenoxy)ethyl]amino}-2,3-dihydro-1H-inden-5-yl)acrylamide |
| 293 | (2E)-N-hydroxy-3-(1-{[2-(1H-indol-3-yl)ethyl][2-(4- |
| | methylphenoxy)ethyl]amino}-2,3-dihydro-1H-inden-5-yl)acrylamide |
| 294 | (2E)-N-hydroxy-3-(1-{(3-hydroxybenzyl)[2-(1H-indol-3-yl)ethyl]amino}-2,3- |
| | dihydro-1H-inden-5-yl)acrylamide |
| 295 | (2E)-N-hydroxy-3-{1-[[2-(1H-indol-3-yl)ethyl](3-methylbenzyl)amino]-2,3- |
| | dihydro-1H-inden-5-yl}acrylamide |
| 296 | (2E)-N-hydroxy-3-(1-{(4-hydroxybenzyl)[2-(1H-indol-3-yl)ethyl]amino}-2,3- |
| | dihydro-1H-inden-5-yl)acrylamide |
| 297 | (2E)-3-(1-{[4-(acetylamino)benzyl][2-(1H-indol-3-yl)ethyl]amino}-2,3- |
| | dihydro-1H-inden-5-yl)-N-hydroxyacrylamide |
| 298 | (2E)-3-((1S)-1-{ethyl[2-(1H-indol-3-yl)ethyl]amino}-2,3-dihydro-1H-inden-5- |
| | yl)-N-hydroxyacrylamide |
| 299 | (2E)-3-((1R)-1-{ethyl[2-(1H-indol-3-yl)ethyl]amino}-2,3-dihydro-1H-inden-5- |
| | yl )-N-hydroxyacrylamide |
| 300 | (2E)-N-hydroxy-3-{(1S)-1-[[2-(1H-indol-3-yl)ethyl](1H-pyrrol-2- |
| | ylmethyl)amino]-2,3-dihydro-1H-inden-5-yl}acrylamide |
| 301 | (2E)-N-hydroxy-3-(1-{[2-(1H-indol-3-yl)ethyl][(1-methyl-1H-pyrrol-2- |
| | yl)methyl]amino}-2,3-dihydro-1H-inden-5-yl)acrylamide |
| 302 | (2E)-N-hydroxy-3-{(1R)-1-[[2-(1H-indol-3-yl)ethyl](1H-pyrrol-2- |
| | ylmethyl)amino]-2,3-dihydro-1H-inden-5-yl}acrylamide |
| 303 | (2E)-N-hydroxy-3-((1S)-1-{(3-hydroxypropyl)[2-(4- |
| | methoxyphenyl)ethyl]amino)-2,3-dihydro-1H-inden-5-yl)acrylamide |
| 304 | (2E)-N-hydroxy-3-((1R)-1-{(3-hydroxypropyl)[2-(4- |
| | methoxyphenyl)ethyl]amino}-2,3-dihydro-1H-inden-5-yl)acrylamide |
| 305 | (2E)-N-hydroxy-3-{1-[[2-(1H-indol-3-yl)ethyl](4-methoxybenzyl)amino]-2,3- |
| | dihydro-1H-inden-5-yl}acrylamide |
| 306 | (2E)-3-(1-{(4-ethoxybenzyl)[2-(1H-indol-3-yl)ethyl]amino}-2,3-dihydro-1H- |
| | inden-5-yl)-N-hydroxyacrylamide |
| 307 | (2E)-N-hydroxy-3-{1-[[2-(1H-indol-3-yl)ethyl](1,3-thiazol-2-ylmethyl)amino]- |
| | 2,3-dihydro-1H-inden-5-yl}acrylamide |
| 308 | (2E)-N-hydroxy-3-[1-([2-(1H-indol-3-yl)ethyl]{[1-(methylsulfonyl)-1H-pyrrol- |
| | 2-yl]methyl}amino)-2,3-dihydro-1H-inden-5-yl]acrylamide |
| 309 | (2E)-N-hydroxy-3-(1-{[4-(2-hydroxyethoxy)benzyl][2-(1H-indol-3- |
| | yl)ethyl]amino}-2,3-dihydro-1H-inden-5-yl)acrylamide |
| 310 | (2E)-3-(1-{[(4-cyano-1H-pyrrol-2-yl)methyl][2-(1H-indol-3-yl)ethyl]amino}- |
| | 2,3-dihydro-1H-inden-5-yl)-N-hydroxyacrylamide |
| 311 | (2E)-N-hydroxy-3-(1-{(1H-imidazol-2-ylmethyl)[2-(1H-indol-3- |
| | yl)ethyl]amino}-2,3-dihydro-1H-inden-5-yl)acrylamide |
| 312 | 5-({{5-[(1E)-3-(hydroxyamino)-3-oxoprop-1-en-1-yl]-2,3-dihydro-1H-inden- |
| | 1-yl}[2-(1H-indol-3-yl)ethyl]amino}methyl)-N-methyl-1H-pyrrole-2- |
| | carboxamide |
| 313 | (2E)-N-hydroxy-3-(1-{[2-(1H-indol-3-yl)ethyl][4-(methylthio)benzyl]amino}- |
| | 2,3-dihydro-1H-inden-5-yl)acrylamide |
| 314 | 5-({{5-[(1E)-3-(hydroxyamino)-3-oxoprop-1-en-1-yl]-2,3-dihydro-1H-inden- |
| | 1-yl}[2-(1H-indol-3-yl)ethyl]amino}methyl)-N,N-dimethyl-1H-pyrrole-2- |
| | carboxamide |
| 315 | (2E)-3-((1S)-1-{[4-(acetylamino)benzyl][2-(1H-indol-3-yl)ethyl]amino}-2,3- |
| | dihydro-1H-inden-5-yl)-N-hydroxyacrylamide |
| 316 | (2E)-3-((1R)-1-{[4-(acetylamino)benzyl][2-(1H-indol-3-yl)ethyl]amino}-2,3- |
| | dihydro-1H-inden-5-yl)-N-hydroxyacrylamide |
| 317 | (2E)-N-hydroxy-3-[1-([2-(1H-indol-3-yl)ethyl]{[5-(morpholin-4-ylcarbonyl)- |
| | 1H-pyrrol-2-yl]methyl}amino)-2,3-dihydro-1H-inden-5-yl]acrylamide |
| 318 | (2E)-N-hydroxy-3-(1-{[2-(1H-indol-3-yl)ethyl][(4-methyl-1H-imidazol-5- |
| | yl)methyl]amino}-2,3-dihydro-1H-inden-5-yl)acrylamide |
| 319 | (2E)-N-hydroxy-3-{1-[[2-(1H-indol-3-yl)ethyl](1H-indol-3-ylmethyl)amino]- |
| | 2,3-dihydro-1H-inden-5-yl}acrylamide |
| 320 | (2E)-3-(1-{{4-[2-(diethylamino)ethoxy]benzyl}[2-(1H-indol-3- |
| | yl)ethyl]amino}-2,3-dihydro-1H-inden-5-yl)-N-hydroxyacrylamide |
| 321 | 4-({{5-[(1E)-3-(hydroxyamino)-3-oxoprop-1-en-1-yl]-2,3-dihydro-1H-inden- |
| | 1-yl}[2-(1H-indol-3-yl)ethyl]amino}methyl)-N,N-dimethyl-1H-pyrrole-2- |
| | carboxamide |
| 322 | (2E)-N-hydroxy-3-(1-{[2-(1H-indol-3-yl)ethyl][4-(2- |
| | methoxyethoxy)benzyl]amino}-2,3-dihydro-1H-inden-5-yl)acrylamide |
| 323 | 4-({{5-[(1E)-3-(hydroxyamino)-3-oxoprop-1-en-1-yl]-2,3-dihydro-1H-iden- |
| | 1-yl}[2-(1H-indol-3-yl)ethyl]amino}methyl)-N-methyl-1H-pyrrole-2- |
| | carboxamide |
| 324 | 5-({{(1R)-5-[(1E)-3-(hydroxyamino)-3-oxoprop-1-en-1-yl]-2,3-dihydro-1H- |
| | inden-1-yl}[2-(1H-indol-3-yl)ethyl]amino}methyl)-N-methyl-1H-pyrrole-2- |
| | carboxamide |
| 325 | 5-({{(1S)-5-[(1E)-3-(hydroxyamino)-3-oxoprop-1-en-1-yl]-2,3-dihydro-1H- |
| | inden-1-yl}[2-(1H-indol-3-yl)ethyl]amino}methyl)-N-methyl-1H-pyrrole-2- |
| | carboxamide |
| 326 | 5-({{(1R)-5-[(1E)-3-(hydroxyamino)-3-oxoprop-1-en-1-yl]-2,3-dihydro-1H- |
| | inden-1-yl}[2-(1H-indol-3yl)ethyl]amino}methyl)-N,N-dimethyl-1H-pyrrole- |
| | 2-carboxamide |
| 327 | 5-({{(1S)-5-[(1E)-3-(hydroxyamino)-3-oxoprop-1-en-1-yl]-2,3-dihydro-1H- |
| | inden-1-yl}[2-(1H-indol-3-yl)ethyl]amino}methyl)-N,N-dimethyl-1H-pyrrole- |
| | 2-carboxamide |
| 328 | (2E)-N-hydroxy-3-(1-{[2-(5-hydroxy-1H-indol-3-yl)ethyl]amino}-2,3-dihydro- |
| | 1H-inden-5-yl)acrylamide |
| 329 | (2E)-N-hydroxy-3-{1-[(2-hydroxyethyl)(2-pyridin-3-ylethyl)amino]-2,3- |
| | dihydro-1H-inden-5-yl}acrylamide |
| 330 | (2E)-N-hydroxy-3-(1-{(2-hydroxyethyl)[2-(5-hydroxy-1H-indol-3- |
| | yl)ethyl]amino}-2,3-dihydro-1H-inden-5-yl)acrylamide |
| 331 | (2E)-N-hydroxy-3-(1-{(2-hydroxyethyl)[2-(5-hydroxy-1H-indol-3- |
| | yl)ethyl]amino}-2,3-dihydro-1H-inden-5-yl)acrylamide |
| 332 | (2E)-3-{1-[(1-benzothien-3-ylmethyl)amino]-2,3-dihydro-1H-inden-5-yl}-N- |
| | hydroxyacrylamide |
| 333 | (2E)-3-(1-{[(5-chloro-1-benzothien-3-yl)methyl]amino}-2,3-dihydro-1H- |
| | inden-5-yl)-N-hydroxyaclylamide |
| 334 | (2E)-N-hydroxy-3-[1-({[6-(trifluoromethyl)pyridin-3-yl]methyl}amino)-2,3- |
| | dihydro-1H-inden-5-yl]acrylamide |
| 335 | (2E)-3-{1-[(1-benzothien-3-ylmethyl)(2-hydroxyethyl)amino]-2,3-dihydro |
| | 1H-inden-5-yl}-N-hydroxyacrylamide |
| 336 | (2E)-3-{1-[[(5-chloro-1-benzothien-3-yl)methyl](2-hydroxyethyl)amino]-2,3- |
| | dihydro-1H-inden-5-yl}-N-hydroxyacrylamide |
| 337 | (2E)-N-hydroxy-3-[1-((2-hydroxyethyl){[6-(trifluoromethyl)pyridin-3- |
| | yl]methyl}amino)-2,3-dihydro-1H-inden-5-yl]acrylamide |
| 338 | (2E)-N-hydroxy-3-{1-[(2-pyridin-4-ylethyl)amino]-2,3-dihydro-1H-inden-5- |
| | yl}acrylamide |
| 339 | (2E)-N-hydroxy-3-{1-[(2-pyridin-2-ylethyl)amino]-2,3-dihydro-1H-inden-5- |
| | yl}acrylamide |
| 340 | (2E)-N-hydroxy-3-{1-[(pyridin-2-ylmethyl)amino]-2,3-dihydro-1H-inden-5- |
| | yl}acrylamide |
| 341 | (2E)-N-hydroxy-3-{1-[(2-hydroxyethyl)(2-pyridin-2-ylethyl)amino]-2,3- |
| | dihydro-1H-inden-5-yl}acrylamide |
| 342 | (2E)-N-hydroxy-3-{1-[(2-hydroxyethyl)(2-pyridin-4-ylethyl)amino]-2,3- |
| | dihydro-1H-inden-5-yl}acrylamide |
| 343 | (2E)-N-hydroxy-3-{1-[(2-hydroxyethyl)(pyridin-2-ylmethyl)amino]-2,3- |
| | dihydro-1H-inden-5-yl}acrylamide |
| 344 | (2E)-N-hydroxy-3-(1-{[(5-methylpyrazin-2-yl)methyl]amino}-2,3-dihydro- |
| | 1H-inden-5-yl)acrylamide |
| 345 | (2E)-N-hydroxy-3-(1-{(2-hydroxyethyl)[(5-methylpyrazin-2- |
| | yl)methyl]amino}-2,3-dihydro-1H-inden-5-yl)acrylamide |
| 346 | (2E)-N-hydroxy-3-(1-{[(5-methyl-2-furyl)methyl]amino}-2,3-dihydro-1H- |
| | inden-5-yl)acrylamide |
| 347 | (2E)-3-{1-[(2-furylmethyl)amino]-2,3-dihydro-1H-inden-5-yl}-N- |
| | hydroxyacrylamide |
| 348 | (2E)-3-(1-{[2-(3,5-dimethylisoxazol-4-yl)ethyl]amino}-2,3-dihydro-1H- |
| | inden-5-yl)-N-hydroxyacrylamide |
| 349 | (2E)-3-{1-[(2-furylmethyl)(2-hydroxyethyl)amino]-2,3-dihydro-1H-inden-5- |
| | yl}-N-hydroxyacrylamide |
| 350 | (2E)-3-{(1S)-1-[[2-(2,5-dimethoxyphenyl)ethyl](2-hydroxyethyl)amino]-2,3- |
| | dihydro-1H-inden-5-yl}-N-hydroxyacrylamide |
| 351 | (2E)-3-{(1R)-1-[[2-(2,5-dimethoxyphenyl)ethyl](2-hydroxyethyl)amino]-2,3- |
| | dihydro-1H-inden-5-yl}-N-hydroxyacrylamide |
| 352 | (2E)-N-hydroxy-3-(1-{(2-hydroxyethyl)[(5-methyl-2-furyl)methyl]amino}-2,3- |
| | dihydro-1H-inden-5-yl)acrylamide |
| 353 | (2E)-N-hydroxy-3-(1-{[2-(1H-indol-3-yl)ethyl][4- |
| | (methylsulfonyl)benzyl]amino}-2,3-dihydro-1H-inden-5-yl)acrylamide |
| 354 | (2E)-N-hydroxy-3-{1-[[2-(1H-indol-3-yl)ethyl](4-methylbenzyl)amino]-2,3- |
| | dihydro-1H-inden-5-yl}acrylamide |
| 355 | (2E)-3-(1-{(4-chlorobenzyl)[2-(1H-indol-3-yl)ethyl]amino}-2,3-dihydro-1H- |
| | inden-5-yl)-N-hydroxyacrylamide |
| 356 | N-ethyl-4-({{5-[(1E)-3-(hydroxyamino)-3-oxoprop-1-en-1-yl]-2,3-dihydro- |
| | 1H-inden-1-yl}[2-(1H-indol-3-yl)ethyl]amino}methyl)benzamide |
| 357 | 4-({{5-[(1E)-3-(hydroxyamino)-3-oxoprop-1-en-1-yl]-2,3-dihydro-1H-inden- |
| | 1-yl}[2-(1H-indol-3-yl)ethyl]amino}methyl)-N-methylbenzamide |
| 358 | (2E)-N-hydroxy-3-[1-([2-(1H-indol-3-yl)ethyl]{4- |
| | [(methylsulfonyl)amino]benzyl}amino)-2,3-dihydro-1H-inden-5- |
| | yl]acrylamide |
| 359 | (2E)-N-hydroxy-3-{1-[[2-(1H-indol-3-yl)ethyl](4- |
| | {[(methylsulfonyl)amino]methyl}benzyl)amino]-2,3-dihydro-1H-inden-5- |
| | yl}acrylamide |
| 360 | (2E)-N-hydroxy-3-[(1S)-1-([2-(1H-indol-3-yl)ethyl]{4- |
| | [(methylsulfonyl)amino]benzyl}amino)-2,3-dihydro-1H-inden-5- |
| | yl]acrylamide |
| 361 | (2E)-N-hydroxy-3-[(1R)-1-([2-(1H-indol-3-yl)ethyl]{4- |
| | [(methylsulfonyl)amino]benzyl}amino)-2,3-dihydro-1H-inden-5- |
| | yl]acrylamide |
| 362 | (2E)-3-(1-{({4-[2-(dimethylamino)ethoxy]phenyl}sulfonyl)[2-(1H-indol-3- |
| | yl)ethyl]amino}-2,3-dihydro-1H-inden-5-yl)-N-hydroxyacrylamide |
| 363 | (2E)-N-hydroxy-3-(1-{{[4-(2-hydroxyethoxy)phenyl]sulfonyl}[2-(1H-indol-3- |
| | yl)ethyl]amino}-2,3-dihydro-1H-inden-5-yl)acrylamide |
| 364 | (2E)-3-(1-{({4-[2-(dimethylamino)ethoxy]phenyl}sulfonyl)[2-(6-methoxy-1H- |
| | indol-3-yl)ethyl]amino}-2,3-dihydro-1H-inden-5-yl)-N-hydroxyacrylamide |
| 365 | (2E)-3-{1-[[2-(5-chloro-1H-indol-3-yl)ethyl]({4-[2- |
| | (dimethylamino)ethoxy]phenyl}sulfonyl)amino]-2,3-dihydro-1H-inden-5-yl}- |
| | N-hydroxyacrylamide |
| 366 | (2E)-3-(1-{({4-[2-(dimethylamino)ethoxy]phenyl}sulfonyl)[2-(2- |
| | methylphenyl)ethyl]amino}-2,3-dihydro-1H-inden-5-yl)-N- |
| | hydroxyacrylamide |
| 367 | (2E)-3-(1-{[(4-{[2-(dimethylamino)ethyl]amino}phenyl)sulfonyl][2-(1H-indol- |
| | 3-yl)ethyl]amino}-2,3-dihydro-1H-inden-5-yl)-N-hydroxyacrylamide |
| 368 | (2E)-N-hydroxy-3-(1-{(2-hydroxyethyl)[2-(1H-indol-1-yl)ethyl]amino}-2,3- |
| | dihydro-1H-inden-5-yl)acrylamide |
| 369 | (2E)-N-hydroxy-3-(1-{(3-hydroxypropyl)[2-(1H-indol-1-yl)ethyl]amino}-2,3- |
| | dihydro-1H-inden-5-yl)acrylamide |
| 370 | (2E)-N-hydroxy-3-(1-{(2-hydroxyethyl)[2-(3-methyl-1H-indol-1- |
| | yl)ethyl]amino}-2,3-dihydro-1H-inden-5-yl)acrylamide |
| 371 | (2E)-N-hydroxy-3-(1-{(2-hydroxyethyl)[2-(2-methyl-1H-indol-1- |
| | yl)ethyl]amino}-2,3-dihydro-1H-inden-5-yl)acrylamide |
| 372 | (2E)-3-{1-[[2-(2,3-dimethyl-1H-indol-1-yl)ethyl](2-hydroxyethyl)amino]-2,3- |
| | dihydro-1H-inden-5-yl}-N-hydroxyacrylamide |
| 373 | (2E)-3-(1-{ethyl[2-(1H-indol-1-yl)ethyl]amino}-2,3-dihydro-1H-inden-5-yl)- |
| | N-hydroxyacrylamide |
| 374 | (2E)-N-hydroxy-3-{1-[[2-(1H-indol-1-yl)ethyl](1H-pyrrol-2-ylmethyl)amino]- |
| | 2,3-dihydro-1H-inden-5-yl}acrylamide |
| 375 | N-hydroxy-3-(1-{(2-hydroxyethyl)[2-(1H-indol-1-yl)ethyl]amino}-2,3- |
| | dihydro-1H-inden-5-yl)propanamide |
| 376 | (2E)-3-{1-[[2-(6-fluoro-2-methyl-1H-indol-1-yl)ethyl](2- |
| | hydroxyethyl)amino]-2,3-dihydro-1H-inden-5-yl}-N-hydroxyacrylamide |
| 377 | (2E)-N-hydroxy-3-(1-{(2-hydroxyethyl)[2-(1H-indol-1-yl)ethyl]amino}-2,3- |
| | dihydro-1H-inden-5-yl)but-2-enamide |
| 378 | (2E)-3-(1-{(cyclopropylmethyl)[2-(1H-indol-1-yl)ethyl]amino}-2,3-dihydro- |
| | 1H-inden-5-yl)-N-hydroxyacrylamide |
| 379 | (2E)-N-hydroxy-3-(1-{(2-hydroxyethyl)[2-(5-methoxy-1H-indol-1- |
| | yl)ethyl]amino}-2,3-dihydro-1H-inden-5-yl)acrylamide |
| 380 | (2E)-N-hydroxy-3-(1-{(2-hydroxyethyl)[2-(6-methoxy-1H-indol-1- |
| | yl)ethyl]amino}-2,3-dihydro-1H-inden-5-yl)acrylamide |
| 381 | (2E)-3-(1-{[4-(acetylamino)benzyl][2-(1H-indol-1-yl)ethyl]amino}-2,3- |
| | dihydro-1H-inden-5-yl)-N-hydroxyacrylamide |
| 382 | (2E)-N-hydroxy-3-(1-{[(1S)-2-hydroxy-1-(1H-indol-1-ylmethyl)ethyl]amino}- |
| | 2,3-dihydro-1H-inden-5-yl)acrylamide |
| 383 | (2E)-N-hydroxy-3-{1-[[(1S)-2-hydroxy-1-(1H-indol-1- |
| | ylmethyl)ethyl](methyl)amino]-2,3-dihydro-1H-inden-5-yl}acrylamide |
| 384 | (2E)-N-hydroxy-3-(1-{[4-(2-hydroxyethoxy)benzyl][2-(1H-indol-1- |
| | yl)ethyl]amino}-2,3-dihydro-1H-inden-5-yl)acrylamide |
| 385 | (2E)-N-hydroxy-3-(1-{[2-(1H-indol-1-yl)ethyl][4-(2- |
| | methoxyethoxy)benzyl]amino}-2,3-dihydro-1H-inden-5-yl)acrylamide |
| 386 | (2E)-3-(1-{{4-[2-(dimethylamino)ethoxy]benzyl}[2-(1H-indol-1- |
| | yl)ethyl]amino}-2,3-dihydro-1H-inden-5-yl)-N-hydroxyacrylamide |
| 387 | 5-({{5-[(1E)-3-(hydroxyamino)-3-oxoprop-1-en-1-yl]-2,3-dihydro-1H-inden- |
| | 1-yl}[2-(1H-indol-1-yl)ethyl]amino)methyl)-N-methyl-1H-pyrrole-2- |
| | carboxamide |
| 388 | 5-({{5-[(1E)-3-(hydroxyamino)-3-oxoprop-1-en-1-yl]-2,3-dihydro-1H-inden- |
| | 1-yl}[2-(1H-indol-1-yl)ethyl]amino}methyl)-N,N-dimethyl-1H-pyrrole-2- |
| | carboxamide |
| 389 | (2E)-N-hydroxy-3-[1-([2-(1H-indol-1-yl)ethyl]{[5-(morpholin-4-ylcarbonyl)- |
| | 1H-pyrrol-2-yl]methyl}amino)-2,3-dihydro-1H-inden-5-yl]acrylamide |
| 390 | 5-({{5-[(1E)-3-(hydroxyamino)-3-oxoprop-1-en-1-yl]-2,3-dihydro-1H-inden- |
| | 1-yl}[2-(1H-indol-1-yl)ethyl]amino}methyl)-N,N-dimethyl-1H-pyrrole-3- |
| | carboxamide |
| 391 | (2E)-N-hydroxy-3-(1-{[2-(1H-indol-1-yl)ethyl][(1-methyl-1H-pyrrol-2- |
| | yl)methyl]amino}-2,3-dihydro-1H-inden-5-yl)acrylamide |
| 392 | (2E)-3-(1-{[2-(acetylamino)ethyl][2-(1H-indol-1-yl)ethyl]amino}-2,3-dihydro- |
| | 1H-inden-5-yl)-N-hydroxyacrylamide |
| 393 | (2E)-N-hydroxy-3-[1-([2-(1H-indol-1-yl)ethyl]{2- |
| | [(methylsulfonyl)amino]ethyl}amino)-2,3-dihydro-1H-inden-5-yl]acrylamide |
| 394 | N-hydroxy-3-(1-{(2-hydroxyethyl)[2-(1H-indol-3-yl)ethyl]amino}-2,3- |
| | dihydro-1H-inden-5-yl)butanamide |
| 395 | N-hydroxy-3-{1-[[2-(1H-indol-3-yl)ethyl](1H-pyrrol-2-ylmethyl)amino]-2,3- |
| | dihydro-1H-inden-5-yl}butanamide |
| 396 | N-hydroxy-3-(1-{(2-hydroxyethyl)[2-(1-methyl-1H-indol-3-yl)ethyl]amino}- |
| | 2,3-dihydro-1H-inden-5-yl)butanamide |
| 397 | (2E)-3-{1-[[2-(1,2-dimethyl-1H-indol-3-yl)ethyl](2-hydroxyethyl)amino]-2,3- |
| | dihydro-1H-inden-5-yl}-N-hydroxyacrylamide |
| 398 | (2E)-N-hydroxy-3-(1-{(2-hydroxyethyl)[2-(6-methoxy-1-methyl-1H-indol-3- |
| | yl)ethyl]amino}-2,3-dihydro-1H-inden-5-yl)acrylamide |
| 399 | (2E)-N-hydroxy-3-(1-{(2-hydroxyethyl)[2-(1H-indol-2-yl)ethyl]amino}-2,3- |
| | dihydro-1H-inden-5-yl)acrylamide |
| 400 | (2E)-N-hydroxy-3-{1-[[2-(1H-indol-2-yl)ethyl](1H-pyrrol-2-ylmethyl)amino]- |
| | 2,3-dihydro-1H-inden-5-yl)acrylamide |
| 401 | (2E)-N-hydroxy-3-(1-{(2-hydroxyethyl)[2-(5-methoxy-1H-indol-2- |
| | yl)ethyl]amino}-2,3-dihydro-1H-inden-5-yl)acrylamide |
| 402 | (2E)-N-hydroxy-3-(1-{(2-hydroxyethyl)[2-(6-methoxy-1H-indol-2- |
| | yl)ethyl]amino}-2,3-dihydro-1H-inden-5-yl)acrylamide |
| 403 | (2E)-3-(1-{[4-(aminosulfonyl)benzyl][2-(1H-indol-3-yl)ethyl]amino}-2,3- |
| | dihydro-1H-inden-5-yl)-N-hydroxyacrylamide |
| 404 | (2E)-N-hydroxy-3-[1-([2-(1H-indol-3-yl)ethyl]{4- |
| | [(methylamino)sulfonyl]benzyl}amino)-2,3-dihydro-1H-inden-5- |
| | yl]acrylamide |
| 405 | (2E)-3-(1-{{4-[(acetylamino)sulfonyl]benzyl}[2-(1H-indol-3-yl)ethyl]amino}- |
| | 2,3-dihydro-1H-inden-5-yl)-N-hydroxyacrylamide |
| 406 | (2E)-3-(1-{[3-(aminosulfonyl)benzyl][2-(1H-indol-3-yl)ethyl]amino}-2,3- |
| | dihydro-1H-inden-5-yl)-N-hydroxyacrylamide |
| 407 | (2E)-N-hydroxy-3-[1-([2-(1H-indol-3-yl)ethyl]{3- |
| | [(methylamino)sulfonyl]benzyl}amino)-2,3-dihydro-1H-inden-5- |
| | yl]acrylamide |
| 408 | (2E)-3-(1-{{3-[(acetylamino)sulfonyl]benzyl}[2-(1H-indol-3-yl)ethyl]amino}- |
| | 2,3-dihydro-1H-inden-5-yl)-N-hydroxyacrylamide |
| 409 | (2E)-N-hydroxy-3-{1-[[2-(1H-indol-3-yl)ethyl](1H-indol-6-ylmethyl)amino]- |
| | 2,3-dihydro-1H-inden-5-yl}acrylamide |
| 410 | (2E)-3-(1-{(1H-benzimidazol-6-ylmethyl)[2-(1H-indol-3-yl)ethyl]amino}-2,3- |
| | dihydro-1H-inden-5-yl)-N-hydroxyacrylamide |
| 411 | (2E)-N-hydroxy-3-{1-[[2-(1H-indol-3-yl)ethyl](1H-indol-5-ylmethyl)amino]- |
| | 2,3-dihydro-1H-inden-5-yl}acrylamide |
| 412 | (2E)-3-{1-[[2-(4,5-dimethyl-1H-pyrrol-3-yl)ethyl](2-hydroxyethyl)amino]-2,3- |
| | dihydro-1H-inden-5-yl}-N-hydroxyacrylamide |
| 413 | (2E)-N-hydroxy-3-(1-{(2-hydroxyethyl)[2-(2,4,5-trimethyl-1H-pyrrol-3- |
| | yl)ethyl]amino}-2,3-dihydro-1H-inden-5-yl)acrylamide |
| 414 | (2E)-N-hydroxy-3-(1-{(2-hydroxyethyl)[2-(4-morpholin-4- |
| | ylphenyl)ethyl]amino}-2,3-dihydro-1H-inden-5-yl)acrylamide |
| 415 | (2E)-N-hydroxy-3-(1-{(2-hydroxyethyl)[2-(4-piperazin-1- |
| | ylphenyl)ethyl]amino}-2,3-dihydro-1H-inden-5-yl)acrylamide |
| 416 | (2E)-N-hydroxy-3-[1-((2-hydroxyethyl){2-[4-(4-methylpiperazin-1- |
| | yl)phenyl]ethyl}amino)-2,3-dihydro-1H-inden-5-yl]acrylamide |
| 417 | (2E)-3-{1-[(2-{4-[2-(dimethylamino)ethoxy]phenyl}ethyl)(2- |
| | hydroxyethyl)amino]-2,3-dihydro-1H-inden-5-yl}-N-hydroxyacrylamide |
| 418 | (2E)-N-hydroxy-3-{1-[(2-hydroxyethyl)(2-{4-[2- |
| | (methylamino)ethoxy]phenyl}ethyl)amino]-2,3-dihydro-1H-inden-5- |
| | yl}acrylamide |
| 419 | (2E)-N-hydroxy-3-(1-{(2-hydroxyethyl)[2-(3-methyl-1H-indol-2- |
| | yl)ethyl]amino}-2,3-dihydro-1H-inden-5-yl)acrylamide |
| 420 | (2E)-3-{1-[[2-(1H-benzimidazol-2-yl)ethyl](2-hydroxyethyl)amino]-2,3- |
| | dihydro-1H-inden-5-yl}-N-hydroxyacrylamide |
| 421 | (2E)-N-hydroxy-3-(1-{(2-hydroxyethyl)[2-(5-methoxy-1H-benzimidazol-2- |
| | yl)ethyl]amino}-2,3-dihydro-1H-inden-5-yl)acrylamide |
| 422 | (2E)-N-hydroxy-3-(1-{(2-hydroxyethyl)[2-(5-methyl-1H-benzimidazol-2- |
| | yl)ethyl]amino}-2,3-dihydro-1H-inden-5-yl)acrylamide |
| 423 | (2E)-3-{1-[[2-(5,6-difluoro-1H-benzimidazol-2-yl)ethyl](2- |
| | hydroxyethyl)amino]-2,3-dihydro-1H-inden-5-yl}-N-hydroxyacrylamide |
| 424 | (2E)-3-{1-[[2-(1,3-benzoxazol-2-yl)ethyl](2-hydroxyethyl)amino]-2,3- |
| | dihydro-1H-inden-5-yl}-N-hydroxyacrylamide |
| 425 | (2E)-3-{1-[[2-(5-chloro-1,3-benzoxazol-2-yl)ethyl](2-hydroxyethyl)amino]- |
| | 2,3-dihydro-1H-inden-5-yl}-N-hydroxyacrylamide |
| 426 | (2E)-3-{1-[[2-(1,3-benzothiazol-2-yl)ethyl](2-hydroxyethyl)amino]-2,3- |
| | dihydro-1H-inden-5-yl}-N-hydroxyacrylamide |
| 427 | (2E)-3-{1-[[2-(6-chloro-1,3-benzothiazol-2-yl)ethyl](2-hydroxyethyl)amino]- |
| | 2,3-dihydro-1H-inden-5-yl}-N-hydroxyacrylamide |
| 428 | (2E)-3-(1-{(4-{[(dimethylamino)sulfonyl]amino}benzyl)[2-(1H-indol-3- |
| | yl)ethyl]amino}-2,3-dihydro-1H-inden-5-yl)-N-hydroxyacrylamide |
| 429 | (2E)-3-(1-{(4-{[(ethylamino)carbonyl]amino}benzyl)[2-(1H-indol-3- |
| | yl)ethyl]amino}-2,3-dihydro-1H-inden-5-yl)-N-hydroxyacrylamide |
| 430 | (2E)-3-(1-{[4({[(ethylamino)carbonyl]amino}methyl)benzyl][2-(1H-indol-3- |
| | yl)ethyl]amino}-2,3-dihydro-1H-inden-5-yl)-N-hydroxyacrylamide |
| 431 | 4-({{5-[(1E)-3-(hydroxyamino)-3-oxoprop-1-en-1-yl]-2,3-dihydro-1H-inden- |
| | 1-yl}[2-(1H-indol-3-yl)ethyl]amino}methyl)benzamide |
| 432 | (2E)-3-(1-{[4-({[(dimethylamino)sulfonyl]amino}methyl)benzyl][2-(1H-indol- |
| | 3-yl)ethyl]amino}-2,3-dihydro-1H-inden-5-yl)-N-hydroxyacrylamide |
| 433 | (2E)-3-(1-{{4-[(acetylamino)methyl]benzyl}[2-(1H-indol-3-yl)ethyl]amino}- |
| | 2,3-dihydro-1H-inden-5-yl)-N-hydroxyacrylamide |
| 434 | (2E)-N-hydroxy-3-{1-[[2-(1H-indol-3-yl)ethyl](3-{[(2- |
| | methoxyethyl)amino]sulfonyl}benzyl)amino]-2,3-dihydro-1H-inden-5- |
| | yl}acrylamide |
| 435 | (2E)-3-(1-{[3-({[2-(dimethylamino)ethyl]amino}sulfonyl)benzyl][2-(1H-indol- |
| | 3-yl)ethyl]amino}-2,3-dihydro-1H-inden-5-yl)-N-hydroxyacrylamide |
| 436 | (2E)-3-(1-{[3-({[2-(diethylamino)ethyl]amino}sulfonyl)benzyl][2-(1H-indol-3- |
| | yl)ethyl]amino}-2,3-dihydro-1H-inden-5-yl)-N-hydroxyacrylamide |
| 437 | (2E)-N-hydroxy-3-{1-[[2-(1H-indol-3-yl)ethyl](3-{[(2-morpholin-4- |
| | ylethyl)amino]sulfonyl}benzyl)amino]-2,3-dihydro-1H-inden-5-yl}acrylamide |
| 438 | (2E)-N-hydroxy-3-{1-[[2-(1H-indol-3-yl)ethyl](3-{[(2-piperazin-1- |
| | ylethyl)amino]sulfonyl}benzyl)amino]-2,3-dihydro-1H-inden-5-yl}acrylamide |
| 439 | (2E)-N-hydroxy-3-(1-{[2-(1H-indol-3-yl)ethyl][3-({[2-(4-methylpiperazin-1- |
| | yl)ethyl]amino}sulfonyl)benzyl]amino}-2,3-dihydro-1H-inden-5- |
| | yl)acrylamide |
| 440 | (2E)-N-hydroxy-3-{1-[[2-(1H-indol-3-yl)ethyl](4-{[(2- |
| | methoxyethyl)amino]sulfonyl}benzyl)amino]-2,3-dihydro-1H-inden-5- |
| | yl}acrylamide |
| 441 | (2E)-3-(1-{[4-({[2-(dimethylamino)ethyl]amino}sulfonyl)benzyl][2-(1H-indol- |
| | 3-yl)ethyl]amino}-2,3-dihydro-1H-inden-5-yl)-N-hydroxyacrylamide |
| 442 | (2E)-N-hydroxy-3-{1-[[2-(1H-indol-3-yl)ethyl](4-{[(2-morpholin-4- |
| | ylethyl)amino]sulfonyl}benzyl)amino]-2,3-dihydro-1H-inden-5-yl}acrylamide |
| 443 | (2E)-N-hydroxy-3-{1-[[2-(1H-indol-3-yl)ethyl](4-{[(2-piperazin-1- |
| | ylethyl)amino]sulfonyl}benzyl)amino]-2,3-dihydro-1H-inden-5-yl}acrylamide |
| 444 | (2E)-N-hydroxy-3-(1-{[2-(1H-indol-3-yl)ethyl][4-({[2-(4-methylpiperazin-1- |
| | yl)ethyl]amino}sulfonyl)benzyl]amino}-2,3-dihydro-1H-inden-5- |
| | yl)acrylamide |
| 445 | (2E)-3-(1-{[4-({[2-(diethylamino)ethyl]amino}sulfonyl)benzyl][2-(1H-indol-3- |
| | yl)ethyl]amino}-2,3-dihydro-1H-inden-5-yl)-N-hydroxyacrylamide |
| 446 | (2E)-3-(1-{(2-{[(diethylamino)carbonyl]amino}ethyl)[2-(1H-indol-3- |
| | yl)ethyl]amino}-2,3-dihydro-1H-inden-5-yl)-N-hydroxyacrylamide |
| 447 | N-(2-{{5-[(1E)-3-(hydroxyamino)-3-oxoprop-1-en-1-yl]-2,3-dihydro-1H- |
| | inden-1-yl}[2-(1H-indol-3-yl)ethyl]amino}ethyl)morpholine-4-carboxamide |
| 448 | (2E)-3-(1-{(2-{[(dimethylamino)sulfonyl]amino}ethyl)[2-(1H-indol-3- |
| | yl)ethyl]amino}-2,3-dihydro-1H-inden-5-yl)-N-hydroxyacrylamide |
| 449 | (2E)-N-hydroxy-3-{1-[(2-(1H-indol-3-yl)-1- |
| | {[(methylsulfonyl)amino]methyl}ethyl)amino]-2,3-dihydro-1H-inden-5- |
| | yl}acrylamide |
| 450 | (2E)-3-(1-{[2-(acetylamino)-1-(1H-indol-3-ylmethyl)ethyl]amino}-2,3- |
| | dihydro-1H-inden-5-yl)-N-hydroxyacrylamide |
| 451 | (2E)-N-hydroxy-3-(1-{[2-(1H-indol-3-yl)ethyl][4-(2-morpholin-4- |
| | ylethoxy)benzyl]amino}-2,3-dihydro-1H-inden-5-yl)acrylamide |
| 452 | (2E)-3-(1-{{4-[2-(dimethylamino)ethoxy]benzyl}[2-(1H-indol-3- |
| | yl)ethyl]amino}-2,3-dihydro-1H-inden-5-yl)-N-hydroxyacrylamide |
| 453 | (2E)-N-hydroxy-3-(1-{[3-(2-hydroxyethoxy)benzyl][2(1H-indol-3- |
| | yl)ethyl]amino}-2,3-dihydro-1H-inden-5-yl)acrylamide |
| 454 | (2E)-N-hydroxy-3-(1-{[2-(1H-indol-3-yl)ethyl][3-(2-morpholin-4- |
| | ylethoxy)benzyl]amino}-2,3-dihydro-1H-inden-5-yl)acrylamide |
| 455 | (2E)-3-(1-{{3-[2-(dimethylamino)ethoxy]benzyl}[2-(1H-indol-3- |
| | yl)ethyl]amino}-2,3-dihydro-1H-inden-5-yl)-N-hydroxyacrylamide |
| 456 | (2E)-3-(1-{{3-[2-(diethylamino)ethoxy]benzyl}[2-(1H-indol-3- |
| | yl)ethyl]amino}-2,3-dihydro-1H-inden-5-yl)-N-hydroxyacrylamide |
| 457 | (2E)-N-hydroxy-3-(1-{[2-(4-phenyl-1H-1,2,3-triazol-1-yl)ethyl]amino}-2,3- |
| | dihydro-1H-inden-5-yl)acrylamide |
| 458 | (2E)-N-hydroxy-3-(1-{(2-hydroxyethyl)[2-(4-phenyl-1H-1,2,3-triazol-1- |
| | yl)ethyl]amino}-2,3-dihydro-1H-inden-5-yl)acrylamide |
| 459 | (2E)-N-hydroxy-3-{1-[[2-(1H-indol-3-yl)ethyl](2H-tetrazol-5- |
| | ylmethyl)amino]-2,3-dihydro-1H-inden-5-yl}acrylamide |
| 460 | (2E)-3-(1-{[3-(5-fluoro-1H-indol-3-yl)propyl]amino}-2,3-dihydro-1H-inden-5- |
| | yl)-N-hydroxyacrylamide |
| 461 | (2E)-3-{1-[[3-(5-fluoro-1H-indol-3-yl)propyl](2-hydroxyethyl)amino]-2,3- |
| | dihydro-1H-inden-5-yl}-N-hydroxyacrylamide |
| 462 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]-1H-indole-2-carboxamide |
| 463 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]-1-benzofuran-2-carboxamide |
| 464 | (2E)-N-hydroxy-3-(1-{[2-(1H-indol-3-yl)ethyl][(3-methyl-1H-pyrazol-5- |
| | yl)methyl]amino}-2,3-dihydro-1H-inden-5-yl)acrylamide |
| 465 | (2E)-3-(1-{[(3-tert-butyl-1H-pyrazol-5-yl)methyl][2-(1H-indol-3- |
| | yl)ethyl]amino}-2,3-dihydro-1H-inden-5-yl)-N-hydroxyacrylamide |
| 466 | (2E)-3-(1-{[(4-bromo-1H-pyrazol-5-yl)methyl][2-(1H-indol-3-yl)ethyl]amino}- |
| | 2,3-dihydro-1H-inden-5-yl)-N-hydroxyacrylamide |
| 467 | (2E)-N-hydroxy-3-(1-{[2-(1H-indol-3-yl)ethyl][(3-propyl-1H-pyrazol-5- |
| | yl)methyl]amino}-2,3-dihydro-1H-inden-5-yl)acrylamide |
| 468 | (2E)-N-hydroxy-3-{1-[[2-(1H-indol-3-yl)ethyl](1H-pyrazol-5- |
| | ylmethyl)amino]-2,3-dihydro-1H-inden-5-yl}acrylamide |
| 469 | (2E)-3-(1-{[(4-chloro-1-methyl-1H-pyrazol-3-yl)methyl][2-(1H-indol-3- |
| | yl)ethyl]amino}-2,3-dihydro-1H-inden-5-yl)-N-hydroxyacrylamide |
| 470 | (2E)-3-(1-{[(1,3-dimethyl-1H-pyrazol-4-yl)methyl][2-(1H-indol-3- |
| | yl)ethyl]amino}-2,3-dihydro-1H-inden-5-yl)-N-hydroxyacrylamide |
| 471 | (2E)-3-(1-{{[5-chloro-1-methyl-3-(trifluoromethyl)-1H-pyrazol-4- |
| | yl]methyl}[2-(1H-indol-3-yl)ethyl]amino}-2,3-dihydro-1H-inden-5-yl)-N- |
| | hydroxyacrylamide |
| 472 | (2E)-3-(1-{[(5-chloro-1,3-dimethyl-1H-pyrazol-4-yl)methyl][2-(1H-indol-3- |
| | yl)ethyl]amino}-2,3-dihydro-1H-inden-5-yl)-N-hydroxyacrylamide |
| 473 | (2E)-N-hydroxy-3-(1-{[2-(1H-indol-3-yl)ethyl][(1,3,5-trimethyl-1H-pyrazol-4- |
| | yl)methyl]amino}-2,3-dihydro-1H-inden-5-yl)acrylamide |
| 474 | (2E)-N-hydroxy-3-(1-{[2-(1H-indol-3-yl)ethyl][(5-methoxy-1,3-dimethyl-1H- |
| | pyrazol-4-yl)methyl]amino}-2,3-dihydro-1H-inden-5-yl)acrylamide |
| 475 | (2E)-3-(1-{[(1,5-dimethyl-1H-pyrazol-4-yl)methyl][2-(1H-indol-3- |
| | yl)ethyl]amino}-2,3-dihydro-1H-inden-5-yl)-N-hydroxyacrylamide |
| 476 | (2E)-3-(1-{[(3,5-dimethylisoxazol-4-yl)methyl][2-(1H-indol-3- |
| | yl)ethyl]amino}-2,3-dihydro-1H-inden-5-yl)-N-hydroxyacrylamide |
| 477 | (2E)-N-hydroxy-3-{1-[[2-(1H-indol-3-yl)ethyl](1,2,3-thiadiazol-4- |
| | ylmethyl)amino]-2,3-dihydro-1H-inden-5-yl}acrylamide |
| 478 | (2E)-N-hydroxy-3-(1-{(2-hydroxy-2-methylpropyl)[2-(1H-indol-3- |
| | yl)ethyl]amino}-2,3-dihydro-1H-inden-5-yl)acrylamide |
| 479 | (2E)-N-hydroxy-3-{1-[[2-(1H-indol-3-yl)ethyl](1H-indol-3-ylmethyl)amino]- |
| | 2,3-dihydro-1H-inden-5-yl}acrylamide |
| 480 | (2E)-N-hydroxy-3-{1-[[2-(1H-indol-3-yl)ethyl][1H-indol-4-ylmethyl)amino]- |
| | 2,3-dihydro-1H-inden-5-yl}acrylamide |
| 481 | (2E)-N-hydroxy-3-{1-[[2-(1H-indol-3-yl)ethyl](1H-indol-7-ylmethyl)amino]- |
| | 2,3-dihydro-1H-inden-5-yl}acrylamide |
| 482 | (2E)-3-(1-{[2-(2,5-dioxopyrrolidin-1-yl)ethyl][2-(1H-indol-3-yl)ethyl]amino}- |
| | 2,3-dihydro-1H-inden-5-yl)-N-hydroxyacrylamide |
| 483 | (2E)-N-hydroxy-3-{1-[[2-(6-methoxy-1H-indol-3-yl)ethyl](1H-pyrrol-2- |
| | ylmethyl)amino]-2,3-dihydro-1H-inden-5-yl}acrylamide |
| 484 | (2E)-N-hydroxy-3-{1-[[2-(6-methoxy-1H-indol-3-yl)ethyl](1H-pyrrol-2- |
| | ylmethyl)amino]-2,3-dihydro-1H-inden-5-yl}acrylamide |
| 485 | (2E)-N-hydroxy-3-{1-[[2-(6-methoxy-1H-indol-3-yl)ethyl](1H-pyrrol-2- |
| | ylmethyl)amino]-2,3-dihydro-1H-inden-5-yl}acrylamide |
| 486 | (2E)-3-(1-{ethyl[2-(6-methoxy-1H-indol-3-yl)ethyl]amino}-2,3-dihydro-1H- |
| | inden-5-yl)-N-hydroxyacrylamide |
| 487 | (2E)-3-(1-{ethyl[2-(6-methoxy-1H-indol-3-yl)ethyl]amino}-2,3-dihydro-1H- |
| | inden-5-yl)-N-hydroxyacrylamide |
| 488 | (2E)-3-(1-{ethyl[2-(6-methoxy-1H-indol-3-yl)ethyl]amino}-2,3-dihydro-1H- |
| | inden-5-yl)-N-hydroxyacrylamide |
| 489 | (2E)-N-hydroxy-3-{1-[[2-(6-methyl-1H-indol-3-yl)ethyl](1H-pyrrol-2- |
| | ylmethyl)amino]-2,3-dihydro-1H-inden-5-yl}acrylamide |
| 490 | (2E)-N-hydroxy-3-{1-[[2-(6-methyl-1H-indol-3-yl)ethyl](1H-pyrrol-2- |
| | ylmethyl)amino]-2,3-dihydro-1H-inden-5-yl}acrylamide |
| 491 | (2E)-N-hydroxy-3-{1-[[2-(6-methyl-1H-indol-3-yl)ethyl](1H-pyrrol-2- |
| | ylmethyl)amino]-2,3-dihydro-1H-inden-5-yl}acrylamide |
| 492 | (2E)-N-hydroxy-3-(1-{[2-(1H-indazol-1-yl)ethyl]amino}-2,3-dihydro-1H- |
| | inden-5-yl)acrylamide |
| 493 | (2E)-N-hydroxy-3-(1-{[2-(2H-indazol-2-yl)ethyl]amino}-2,3-dihydro-1H- |
| | inden-5-yl)acrylamide |
| 494 | (2E)-N-hydroxy-3-(1-{(2-hydroxyethyl)[2-(1H-indazol- |
| | 1-yl)ethyl]amino}-2,3-dihydro-1H-inden-5-yl)acrylamide |
| 495 | (2E)-3-(1-{[2-(1H-1,2,3-benzotriazol-1-yl)ethyl]amino}-2,3-dihydro-1H- |
| | inden-5-yl)-N-hydroxyacrylamide |
| 496 | (2E)-3-{1-[[2-(1H-1,2,3-benzotriazol-1-yl)ethyl](2-hydroxyethyl)amino]-2,3- |
| | dihydro-1H-inden-5-yl}-N-hydroxyacrylamide |
| 497 | (2E)-N-hydroxy-3-(1-{[2-(1H-indazol-3-yl)ethyl]amino}-2,3-dihydro-1H- |
| | inden-5-yl)acrylamide |
| 498 | (2E)-N-hydroxy-3-(1-{(2-hydroxyethyl)[2-(1H-indazol-3-yl)ethyl]amino}-2,3- |
| | dihydro-1H-inden-5-yl)acrylamide |
| 499 | (2E)-3-(1-{[2-(1H-benzimidazol-1-yl)ethyl]amino}-2,3-dihydro-1H-inden-5- |
| | yl)-N-hydroxyacrylamide |
| 500 | (2E)-3-{1-[[2-(1H-benzimidazol-1-yl)ethyl](2-hydroxyethyl)amino]-2,3- |
| | dihydro-1H-inden-5-yl}-N-hydroxyacrylamide |
| 501 | (2E)-N-hydroxy-3-(1-{[4-(2-hydroxyethoxy)benzyl][2-(1H-indol-3- |
| | yl)ethyl]amino}-2,3-dihydro-1H-inden-5-yl)acrylamide |
| 502 | (2E)-N-hydroxy-3-(1-{[4-(2-hydroxyethoxy)benzyl][2-(1H-indol-3- |
| | yl)ethyl]amino}-2,3-dihydro-1H-inden-5-yl)acrylamide |
| 503 | (2E)-3-(1-{[(1-ethyl-1H-pyrazol-4-yl)methyl][2-(1H-indol-3-yl)ethyl]amino}- |
| | 2,3-dihydro-1H-inden-5-yl)-N-hydroxyacrylamide |
| 504 | (2E)-3-(1-({[4-(acetylamino)phenyl]sulfonyl}[2-(1H-indol-3-yl)ethyl]amino}- |
| | 2,3-dihydro-1H-inden-5-yl)-N-hydroxyacrylamide |
| 505 | (2E)-3-(1-{[(4-aminophenyl)sulfonyl][2-(1H-indol-3-yl)ethyl]amino}-2,3- |
| | dihydro-1H-inden-5-yl)-N-hydroxyacrylamide |
| 506 | (2E)-N-hydroxy-3-[1-([2-(1H-indol-3-yl)ethyl]{3- |
| | [(methylsulfonyl)amino]benzyl}amino)-2,3-dihydro-1H-inden-5- |
| | yl]acrylamide |
| 507 | (2E)-N-hydroxy-3-{1-[[2-(3-methyl-1H-indol-1-yl)ethyl](1H-pyrrol-2- |
| | ylmethyl)amino]-2,3-dihydro-1H-inden-5-yl}acrylamide |
| 508 | (2E)-N-hydroxy-3-(1-{[2-(3-methyl-1H-indol-1-yl)ethyl]amino}-2,3-dihydro- |
| | 1H-inden-5-yl)acrylamide |
| 509 | (2E)-N-hydroxy-3-(1-{[2-(1H-indol-1-yl)ethyl]amino}-2,3-dihydro-1H-inden- |
| | 5-yl)acrylamide |
| 510 | (2E)-N-hydroxy-3-(1-{[2-(5-methoxy-1H-indol-1-yl)ethyl]amino}-2,3- |
| | dihydro-1H-inden-5-yl)acrylamide |
| 511 | (2E)-N-hydroxy-3-(1-{[(4-hydroxyphenyl)sulfonyl][2-(1H-indol-3- |
| | yl)ethyl]amino}-2,3-dihydro-1H-inden-5-yl)acrylamide |
| 512 | (2E)-N-hydroxy-3-(1-{({4-[(2-hydroxyethyl)amino]phenyl}sulfonyl)[2-(1H- |
| | indol-3-yl)ethyl]amino}-2,3-dihydro-1H-inden-5-yl)acrylamide |
| 513 | (2E)-3-(1-{ethyl[2-(1-methyl-1H-indol-3-yl)ethyl]amino}-2,3-dihydro-1H- |
| | inden-5-yl)-N-hydroxyacrylamide |
| 514 | (2E)-3-(1-{ethyl[2-(1-methyl-1H-indol-3-yl)ethyl]amino}-2,3-dihydro-1H- |
| | inden-5-yl)-N-hydroxyacrylamide |
| 515 | (2E)-3-(1-{ethyl[2-(1-methyl-1H-indol-3-yl)ethyl]amino}-2,3-dihydro-1H- |
| | inden-5-yl)-N-hydroxyacrylamide |
| 516 | (2E)-3-(1-{ethyl[2-(6-methyl-1H-indol-3-yl)ethyl]amino}-2,3-dihydro-1H- |
| | inden-5-yl)-N-hydroxyacrylamide |
| 517 | (2E)-3-(1-{ethyl[2-(6-methyl-1H-indol-3-yl)ethyl]amino}-2,3-dihydro-1H- |
| | inden-5-yl)-N-hydroxyacrylamide |
| 518 | (2E)-3-(1-{ethyl[2-(6-methyl-1H-indol-3-yl)ethyl]amino}-2,3-dihydro-1H- |
| | inden-5-yl)-N-hydroxyacrylamide |
| 519 | (2E)-N-hydroxy-3-{1-[[2-(1-methyl-1H-indol-3-yl)ethyl](1H-pyrrol-2- |
| | ylmethyl)amino]-2,3-dihydro-1H-inden-5-yl}acrylamide |
| 520 | (2E)-N-hydroxy-3-{1-[[2-(1-methyl-1H-indol-3-yl)ethyl](1H-pyrrol-2- |
| | ylmethyl)amino]-2,3-dihydro-1H-inden-5-yl}acrylamide |
| 521 | (2E)-N-hydroxy-3-{1-[[2-(1-methyl-1H-indol-3-yl)ethyl](1H-pyrrol-2- |
| | ylmethyl)amino]-2,3-dihydro-1H-inden-5-yl}acrylamide |
| 522 | (2E)-3-{1-[[2-(6-chloro-1H-indol-3-yl)ethyl](1H-pyrrol-2-ylmethyl)amino]- |
| | 2,3-dihydro-1H-inden-5-yl}-N-hydroxyacrylamide |
| 523 | (2E)-3-{1-[[2-(6-chloro-1H-indol-3-yl)ethyl](1H-pyrrol-2-ylmethyl)amino]- |
| | 2,3-dihydro-1H-inden-5-yl}-N-hydroxyacrylamide |
| 524 | (2E)-3-{1-[[2-(6-chloro-1H-indol-3-yl)ethyl](1H-pyrrol-2-ylmethyl)amino]- |
| | 2,3-dihydro-1H-inden-5-yl}-N-hydroxyacrylamide |
| 525 | (2E)-3-{1-[[2-(6-fluoro-1H-indol-3-yl)ethyl](1H-pyrrol-2-ylmethyl)amino]-2,3- |
| | dihydro-1H-inden-5-yl}-N-hydroxyacrylamide |
| 526 | (2E)-3-{1-[[2-(6-fluoro-1H-indol-3-yl)ethyl](1H-pyrrol-2-ylmethyl)amino]-2,3- |
| | dihydro-1H-inden-5-yl}-N-hydroxyacrylamide |
| 527 | (2E)-3-{1-[[2-(6-fluoro-1H-indol-3-yl)ethyl](1H-pyrrol-2-ylmethyl)amino]-2,3- |
| | dihydro-1H-inden-5-yl}-N-hydroxyacrylamide |
| 528 | (2E)-3-(1-{{4-[2-(diethylamino)ethoxy]benzyl}[2-(1H-indol-3- |
| | yl)ethyl]amino}-2,3-dihydro-1H-inden-5-yl)-N-hydroxyacrylamide |
| 529 | (2E)-3-(1-{{4-[2-(diethylamino)ethoxy]benzyl}[2-(1H-indol-3- |
| | yl)ethyl]amino}-2,3-dihydro-1H-inden-5-yl)-N-hydroxyacrylamide |
| 530 | (2E)-3-[1-([2-(6-chloro-1H-indol-3-yl)ethyl]{4-[2- |
| | (diethylamino)ethoxy]benzyl}amino)-2,3-dihydro-1H-inden-5-yl]-N- |
| | hydroxyacrylamide |
| 531 | (2E)-3-(1-([2-(6-chloro-1H-indol-3-yl)ethyl]{4-[2- |
| | (diethylamino)ethoxy]benzyl}amino)-2,3-dihydro-1H-inden-5-yl]-N- |
| | hydroxyacrylamide |
| 532 | (2E)-3-[1-([2-(6-chloro-1H-indol-3-yl)ethyl]{4-[2- |
| | (diethylamino)ethoxy]benzyl}amino)-2,3-dihydro-1H-inden-5-yl]-N- |
| | hydroxyacrylamide |
| 533 | (2E)-3-(1-{{4-[2-(diethylamino)ethoxy]benzyl}[2-(6-fluoro-1H-indol-3- |
| | yl)ethyl]amino}-2,3-dihydro-1H-inden-5-yl)-N-hydroxyacrylamide |
| 534 | (2E)-3-(1-{{4-[2-(diethylamino)ethoxy]benzyl}[2-(6-fluoro-1H-indol-3- |
| | yl)ethyl]amino}-2,3-dihydro-1H-inden-5-yl)-N-hydroxyacrylamide |
| 535 | (2E)-3-(1-{{4-[2-(diethylamino)ethoxy]benzyl}[2-(6-fluoro-1H-indol-3- |
| | yl)ethyl]amino}-2,3-dihydro-1H-inden-5-yl)-N-hydroxyacrylamide |
| 536 | (2E)-3-(1-{{4-[2-(diethylamino)ethoxy]benzyl}[2-(6-methyl-1H-indol-3- |
| | yl)ethyl]amino}-2,3-dihydro-1H-inden-5-yl)-N-hydroxyacrylamide |
| 537 | (2E)-3-(1-{{4-[2-(diethylamino)ethoxy]benzyl}[2-(6-methyl-1H-indol-3- |
| | yl)ethyl]amino}-2,3-dihydro-1H-inden-5-yl)-N-hydroxyacrylamide |
| 538 | (2E)-3-(1-{{4-[2-(diethylamino)ethoxy]benzyl}[2-(6-methyl-1H-indol-3- |
| | yl)ethyl]amino}-2,3-dihydro-1H-inden-5-yl)-N-hydroxyacrylamide |
| 539 | (2E)-3-(1-{{4-[2-(diethylamino)ethoxy]benzyl}[2-(6-methoxy-1H-indol-3- |
| | yl)ethyl]amino}-2,3-dihydro-1H-inden-5-yl)-N-hydroxyacrylamide |
| 540 | (2E)-3-(1-{{4-[2-(diethylamino)ethoxy]benzyl}[2-(6-methoxy-1H-indol-3- |
| | yl)ethyl]amino}-2,3-dihydro-1H-inden-5-yl)-N-hydroxyacrylamide |
| 541 | (2E)-3-(1-{{4-[2-(diethylamino)ethoxy]benzyl}[2-(6-methoxy-1H-indol-3- |
| | yl)ethyl]amino}-2,3-dihydro-1H-inden-5-yl)-N-hydroxyacrylamide |
| 542 | (2E)-3-(1-{{4-[2-(diethylamino)ethoxy]benzyl}[2-(1-methyl-1H-indol-3- |
| | yl)ethyl]amino}-2,3-dihydro-1H-inden-5-yl)-N-hydroxyacrylamide |
| 543 | (2E)-3-(1-{{4-[2-(diethylamino)ethoxy]benzyl}[2-(1-methyl-1H-indol-3- |
| | yl)ethyl]amino}-2,3-dihydro-1H-inden-5-yl)-N-hydroxyacrylamide |
| 544 | (2E)-3-(1-{{4-[2-(diethylamino)ethoxy]benzyl}[2-(1-methyl-1H-indol-3- |
| | yl)ethyl]amino}-2,3-dihydro-1H-inden-5-yl)-N-hydroxyacrylamide |
| 545 | (2E)-3-(1-{[(1-ethyl-1H-pyrazol-4-yl)methyl][2-(1H-indol-3-yl)ethyl]amino}- |
| | 2,3-dihydro-1H-inden-5-yl)-N-hydroxyacrylamide |
| 546 | (2E)-3-(1-{[(1-ethyl-1H-pyrazol-4-yl)methyl][2-(1H-indol-3-yl)ethyl]amino}- |
| | 2,3-dihydro-1H-inden-5-yl)-N-hydroxyacrylamide |
| 547 | (2E)-3-(1-{[(1-ethyl-1H-pyrazol-4-yl)methyl][2-(1-methyl-1H-indol-3- |
| | yl)ethyl]amino}-2,3-dihydro-1H-inden-5-yl)-N-hydroxyacrylamide |
| 548 | (2E)-3-(1-{[(1-ethyl-1H-pyrazol-4-yl)methyl][2-(1-methyl-1H-indol-3- |
| | yl)ethyl]amino}-2,3-dihydro-1H-inden-5-yl)-N-hydroxyacrylamide |
| 549 | (2E)-3-(1-{[(1-ethyl-1H-pyrazol-4-yl)methyl][2-(1-methyl-1H-indol-3- |
| | yl)ethyl]amino}-2,3-dihydro-1H-inden-5-yl)-N-hydroxyacrylamide |
| 550 | (2E)-3-(1-{[(1-ethyl-1H-pyrazol-4-yl)methyl][2-(6-methoxy-1H-indol-3- |
| | yl)ethyl]amino}-2,3-dihydro-1H-inden-5-yl)-N-hydroxyacrylamide |
| 551 | (2E)-3-(1-{[(1-ethyl-1H-pyrazol-4-yl)methyl][2-(6-methoxy-1H-indol-3- |
| | yl)ethyl]amino}-2,3-dihydro-1H-inden-5-yl)-N-hydroxyacrylamide |
| 552 | (2E)-3-(1-{[(1-ethyl-1H-pyrazol-4-yl)methyl][2-(6-methoxy-1H-indol-3- |
| | yl)ethyl]amino}-2,3-dihydro-1H-inden-5-yl)-N-hydroxyacrylamide |
| 553 | (2E)-3-(1-{[2-(6-chloro-1H-indol-3-yl)ethyl][(1-ethyl-1H-pyrazol-4- |
| | yl)methyl]amino}-2,3-dihydro-1H-inden-5-yl)-N-hydroxyacrylamide |
| 554 | (2E)-3-(1-{[2-(6-chloro-1H-indol-3-yl)ethyl][(1-ethyl-1H-pyrazol-4- |
| | yl)methyl]amino}-2,3-dihydro-1H-inden-5-yl)-N-hydroxyacrylamide |
| 555 | (2E)-3-(1-{[2-(6-chloro-1H-indol-3-yl)ethyl][(1-ethyl-1H-pyrazol-4- |
| | yl)methyl]amino}-2,3-dihydro-1H-inden-5-yl)-N-hydroxyacrylamide |
| 556 | (2E)-3-(1-{[(1-ethyl-1H-pyrazol-4-yl)methyl][2-(6-fluoro-1H-indol-3- |
| | yl)ethyl]amino}-2,3-dihydro-1H-inden-5-yl)-N-hydroxyacrylamide |
| 557 | (2E)-3-(1-{[(1-ethyl-1H-pyrazol-4-yl)methyl][2-(6-fluoro-1H-indol-3- |
| | yl)ethyl]amino}-2,3-dihydro-1H-inden-5-yl)-N-hydroxyacrylamide |
| 558 | (2E)-3-(1-{[(1-ethyl-1H-pyrazol-4-yl)methyl][2-(6-fluoro-1H-indol-3- |
| | yl)ethyl]amino}-2,3-dihydro-1H-inden-5-yl)-N-hydroxyacrylamide |
| 559 | (2E)-3-(1-{[(1-ethyl-1H-pyrazol-4-yl)methyl][2-(6-methyl-1H-indol-3- |
| | yl)ethyl]amino}-2,3-dihydro-1H-inden-5-yl)-N-hydroxyacrylamide |
| 560 | (2E)-3-(1-{[(1-ethyl-1H-pyrazol-4-yl)methyl][2-(6-methyl-1H-indol-3- |
| | yl)ethyl]amino}-2,3-dihydro-1H-inden-5-yl)-N-hydroxyacrylamide |
| 561 | (2E)-3-(1-{[(1-ethyl-1H-pyrazol-4-yl)methyl][2-(6-methyl-1H-indol-3- |
| | yl)ethyl]amino}-2,3-dihydro-1H-inden-5-yl)-N-hydroxyacrylamide |
| 562 | (2E)-N-hydroxy-3-(1-{(2-hydroxyethyl)[2-(6-methyl-1H-indol-3- |
| | yl)ethyl]amino}-2,3-dihydro-1H-inden-5-yl)acrylamide |
| 563 | (2E)-N-hydroxy-3-(1-{(2-hydroxyethyl)[2-(6-methyl-1H-indol-3- |
| | yl)ethyl]amino}-2,3-dihydro-1H-inden-5-yl)acrylamide |
| 564 | (2E)-N-hydroxy-3-(1-{(2-hydroxyethyl)[2-(6-methyl-1H-indol-3- |
| | yl)ethyl]amino}-2,3-dihydro-1H-inden-5-yl)acrylamide |
| 565 | (2E)-3-{1-[[2-(6-fluoro-1H-indol-3-yl)ethyl](2-hydroxyethyl)amino]-2,3- |
| | dihydro-1H-inden-5-yl}-N-hydroxyacrylamide |
| 566 | (2E)-3-{1-[[2-(6-fluoro-1H-indol-3-yl)ethyl](2-hydroxyethyl)amino]-2,3- |
| | dihydro-1H-inden-5-yl}-N-hydroxyacrylamide |
| 567 | (2E)-3-{1-[[2-(6-fluoro-1H-indol-3-yl)ethyl](2-hydroxyethyl)amino]-2,3- |
| | dihydro-1H-inden-5-yl}-N-hydroxyacrylamide |
| 568 | (2E)-3-{1-[[2-(6-chloro-1H-indol-3-yl)ethyl](2-hydroxyethyl)amino]-2,3- |
| | dihydro-1H-inden-5-yl}-N-hydroxyacrylamide |
| 569 | (2E)-3-{1-[[2-(6-chloro-1H-indol-3-yl)ethyl](2-hydroxyethyl)amino]-2,3- |
| | dihydro-1H-inden-5-yl}-N-hydroxyacrylamide |
| 570 | (2E)-3-{1-[[2-(6-chloro-1H-indol-3-yl)ethyl](2-hydroxyethyl)amino]-2,3- |
| | dihydro-1H-inden-5-yl}-N-hydroxyacrylamide |
| 571 | (2E)-N-hydroxy-3-(1-{(2-hydroxyethyl)[2-(1-methyl-1H-indol-3- |
| | yl)ethyl]amino}-2,3-dihydro-1H-inden-5-yl)acrylamide |
| 572 | (2E)-N-hydroxy-3-(1-{(2-hydroxyethyl)[2-(1-methyl-1H-indol-3- |
| | yl)ethyl]amino}-2,3-dihydro-1H-inden-5-yl)acrylamide |
| 573 | (2E)-N-hydroxy-3-{1-[[2-(1H-indol-3-yl)ethyl](2- |
| | {[(trifluoromethyl)sulfonyl]amino}ethyl)amino]-2,3-dihydro-1H-inden-5- |
| | yl}acrylamide |
| 574 | (2E)-N-hydroxy-3-(1-{[2-(1H-indol-3-yl)ethyl][(1-methyl-1H-indol-2- |
| | yl)methyl]amino}-2,3-dihydro-1H-inden-5-yl)acrylamide |
| 575 | (2E)-N-hydroxy-3-[1-([2-(1H-indol-3-yl)ethyl]{[4-(2-piperidin-1- |
| | ylethoxy)phenyl]sulfonyl}amino)-2,3-dihydro-1H-inden-5-yl]acrylamide |
| 576 | (2E)-N-hydroxy-3-(1-{[2-(6-methoxy-1H-indol-1-yl)ethyl]amino}-2,3- |
| | dihydro-1H-inden-5-yl)acrylamide |
| 577 | (2E)-3-(1-{[3-(7-chloro-1H-indol-3-yl)propyl]amino}-2,3-dihydro-1H-inden- |
| | 5-yl)-N-hydroxyacrylamide |
| 578 | (2E)-N-hydroxy-3-(1-{[2-(1H-indol-3-yl)ethyl][(3-methyl-1H-pyrazol-4- |
| | yl)methyl]amino}-2,3-dihydro-1H-inden-5-yl)acrylamide |
| 579 | (2E)-3-(1-{[2-(6-fluoro-1H-indol-3-yl)ethyl][(3-methyl-1H-pyrazol-4- |
| | yl)methyl]amino}-2,3-dihydro-1H-inden-5-yl)-N-hydroxyacrylamide |
| 580 | (2E)-3-(1-{[2-(6-chloro-1H-indol-3-yl)ethyl][(3-methyl-1H-pyrazol-4- |
| | yl)methyl]amino}-2,3-dihydro-1H-inden-5-yl)-N-hydroxyacrylamide |
| 581 | (2E)-N-hydroxy-3-(1-{[2-(6-methyl-1H-indol-3-yl)ethyl][(3-methyl-1H- |
| | pyrazol-4-yl)methyl]amino}-2,3-dihydro-1H-inden-5-yl)acrylamide |
| 582 | (2E)-N-hydroxy-3-(1-{[2-(1-methyl-1H-indol-3-yl)ethyl][(3-methyl-1H- |
| | pyrazol-4-yl)methyl]amino}-2,3-dihydro-1H-inden-5-yl)acrylamide |
| 583 | (2E)-N-hydroxy-3-(1-{[2-(6-methoxy-1H-indol-3-yl)ethyl][(3-methyl-1H- |
| | pyrazol-4-yl)methyl]amino}-2,3-dihydro-1H-inden-5-yl)acrylamide |
| 584 | (2E)-3-(1-{[(1,5-dimethyl-1H-pyrazol-4-yl)methyl][2-(6-fluoro-1H-indol-3- |
| | yl)ethyl]amino}-2,3-dihydro-1H-inden-5-yl)-N-hydroxyacrylamide |
| 585 | (2E)-3-(1-{[2-(6-chloro-1H-indol-3-yl)ethyl][(1,5-dimethyl-1H-pyrazol-4- |
| | yl)methyl]amino}-2,3-dihydro-1H-inden-5-yl)-N-hydroxyacrylamide |
| 586 | (2E)-3-(1-{[(1,5-dimethyl-1H-pyrazol-4-yl)methyl][2-(6-methyl-1H-indol-3- |
| | yl)ethyl]amino}-2,3-dihydro-1H-inden-5-yl)-N-hydroxyacrylamide |
| 587 | (2E)-3-(1-{[(1,5-dimethyl-1H-pyrazol-4-yl)methyl][2-(6-methoxy-1H-indol-3- |
| | yl)ethyl]amino}-2,3-dihydro-1H-inden-5-yl)-N-hydroxyacrylamide |
| 588 | (2E)-3-(1-{[(1,5-dimethyl-1H-pyrazol-4-yl)methyl][2-(1-methyl-1H-indol-3- |
| | yl)ethyl]amino}-2,3-dihydro-1H-inden-5-yl)-N-hydroxyacrylamide |
| 589 | (2E)-3-(1-{[(1,3-dimethyl-1H-pyrazol-4-yl)methyl][2-(1-methyl-1H-indol-3- |
| | yl)ethyl]amino}-2,3-dihydro-1H-inden-5-yl)-N-hydroxyacrylamide |
| 590 | (2E)-3-(1-{[(1,3-dimethyl-1H-pyrazol-4-yl)methyl][2-(6-fluoro-1H-indol-3- |
| | yl)ethyl]amino}-2,3-dihydro-1H-inden-5-yl)-N-hydroxyacrylamide |
| 591 | (2E)-3-(1-{[2-(6-chloro-1H-indol-3-yl)ethyl][(1,3-dimethyl-1H-pyrazol-4- |
| | yl)methyl]amino}-2,3-dihydro-1H-inden-5-yl)-N-hydroxyacrylamide |
| 592 | (2E)-3-(1-{[(1,3-dimethyl-1H-pyrazol-4-yl)methyl][2-(6-methyl-1H-indol-3- |
| | yl)ethyl]amino}-2,3-dihydro-1H-inden-5-yl)-N-hydroxyacrylamide |
| 593 | (2E)-3-(1-{[(1,3-dimethyl-1H-pyrazol-4-yl)methyl][2-(6-methoxy-1H-indol-3- |
| | yl)ethyl]amino}-2,3-dihydro-1H-inden-5-yl)-N-hydroxyacrylamide |
| 594 | (2E)-N-hydroxy-3-(1-{[2-(1-methyl-1H-indol-3-yl)ethyl][(1-methyl-1H- |
| | pyrazol-4-yl)methyl]amino}-2,3-dihydro-1H-inden-5-yl)acrylamide |
| 595 | (2E)-3-(1-{[2-(6-fluoro-1H-indol-3-yl)ethyl][(1-methyl-1H-pyrazol-4- |
| | yl)methyl]amino}-2,3-dihydro-1H-inden-5-yl)-N-hydroxyacrylamide |
| 596 | (2E)-3-(1-{[2-(6-chloro-1H-indol-3-yl)ethyl][(1-methyl-1H-pyrazol-4- |
| | yl)methyl]amino}-2,3-dihydro-1H-inden-5-yl)-N-hydroxyacrylamide |
| 597 | (2E)-N-hydroxy-3-(1-{[2-(6-methyl-1H-indol-3-yl)ethyl][(1-methyl-1H- |
| | pyrazol-4-yl)methyl]amino}-2,3-dihydro-1H-inden-5-yl)acrylamide |
| 598 | (2E)-N-hydroxy-3-(1-{[2-(6-methoxy-1H-indol-3-yl)ethyl][(1-methyl-1H- |
| | pyrazol-4-yl)methyl]amino}-2,3-dihydro-1H-inden-5-yl)acrylamide |
| 599 | (2E)-N-hydroxy-3-(1-{[2-(1H-indol-3-yl)ethyl][(1-methyl-1H-pyrazol-4- |
| | yl)methyl]amino}-2,3-dihydro-1H-inden-5-yl)acrylamide |
| 600 | (2E)-3-(1-{[2-(6-fluoro-1H-indol-3-yl)ethyl][(4- |
| | hydroxyphenyl)sulfonyl]amino}-2,3-dihydro-1H-inden-5-yl)-N- |
| | hydroxyacrylamide |
| 601 | (2E)-3-(1-{[2-(6-chloro-1H-indol-3-yl)ethyl][(4- |
| | hydroxyphenyl)sulfonyl]amino}-2,3-dihydro-1H-inden-5-yl)-N- |
| | hydroxyacrylamide |
| 602 | (2E)-N-hydroxy-3-(1-{[(4-hydroxyphenyl)sulfonyl][2-(6-methyl-1H-indol-3- |
| | yl)ethyl]amino}-2,3-dihydro-1H-inden-5-yl)acrylamide |
| 603 | (2E)-N-hydroxy-3-(1-{[(4-hydroxyphenyl)sulfonyl][2-(6-methoxy-1H-indol-3- |
| | yl)ethyl]amino}-2,3-dihydro-1H-inden-5-yl)acrylamide |
| 604 | (2E)-N-hydroxy-3-(1-{[(4-hydroxyphenyl)sulfonyl][2-(1-methyl-1H-indol-3- |
| | yl)ethyl]amino}-2,3-dihydro-1H-inden-5-yl)acrylamide |
| 605 | (2E)-3-(1-{(4-{[(ethylamino)carbonyl]amino}benzyl)[2-(6-fluoro-1H-indol-3- |
| | yl)ethyl]amino}-2,3-dihydro-1H-inden-5-yl)-N-hydroxyacrylamide |
| 606 | (2E)-3-{1-[[2-(6-chloro-1H-indol-3-yl)ethyl](4- |
| | {[(ethylamino)carbonyl]amino}benzyl)amino]-2,3-dihydro-1H-inden-5-yl}-N- |
| | hydroxyacrylamide |
| 607 | (2E)-3-(1-{(4-{[(ethylamino)carbonyl]amino}benzyl)[2-(6-methyl-1H-indol-3- |
| | yl)ethyl]amino}-2,3-dihydro-1H-inden-5-yl)-N-hydroxyacrylamide |
| 608 | (2E)-3-(1-{(4-{[(ethylamino)carbonyl]amino}benzyl)[2-(6-methoxy-1H-indol- |
| | 3-yl)ethyl]amino}-2,3-dihydro-1H-inden-5-yl)-N-hydroxyacrylamide |
| 609 | (2E)-3-(1-{(4-{[(ethylamino)carbonyl]amino}benzyl)[2-(1-methyl-1H-indol-3- |
| | yl)ethyl]amino}-2,3-dihydro-1H-inden-5-yl)-N-hydroxyacrylamide |
| 610 | (2E)-3-(1-{(cyclopropylmethyl)[2-(1-methyl-1H-indol- |
| | 3-yl)ethyl]amino}-2,3-dihydro-1H-inden-5-yl)-N-hydroxyacrylamide |
| 611 | (2E)-3-(1-{(cyclopropylmethyl)[2-(6-fluoro-1H-indol- |
| | 3-yl)ethyl]amino}-2,3-dihydro-1H-inden-5-yl)-N-hydroxyacrylamide |
| 612 | (2E)-3-{1-[[2-(6-chloro-1H-indol-3-yl)ethyl](cyclopropylmethyl)amino]-2,3- |
| | dihydro-1H-inden-5-yl}-N-hydroxyacrylamide |
| 613 | (2E)-3-(1-{(cyclopropylmethyl)[2-(6-methyl-1H-indol- |
| | 3-yl)ethyl]amino}-2,3-dihydro-1H-inden-5-yl)-N-hydroxyacrylamide |
| 614 | (2E)-3-(1-{(cyclopropylmethyl)[2-(6-methoxy-1H-indol-3-yl)ethyl]amino}- |
| | 2,3-dihydro-1H-inden-5-yl)-N-hydroxyacrylamide |
| 615 | (2E)-3-{1-[[2-(6-fluoro-1H-indol-3-yl)ethyl](methyl)amino]-2,3-dihydro-1H- |
| | inden-5-yl}-N-hydroxyacrylamide |
| 616 | (2E)-3-{1-[[2-(6-chloro-1H-indol-3-yl)ethyl](methyl)amino]-2,3-dihydro-1H- |
| | inden-5-yl}-N-hydroxyacrylamide |
| 617 | (2E)-N-hydroxy-3-(1-{methyl[2-(6-methyl-1H-indol-3- |
| | yl)ethyl]amino}-2,3-dihydro-1H-inden-5-yl)acrylamide |
| 618 | (2E)-N-hydroxy-3-{1-[[2-(6-methoxy-1H-indol-3-yl)ethyl](methyl)amino]- |
| | 2,3-dihydro-1H-inden-5-yl}acrylamide |
| 619 | (2E)-3-(1-{[2-(6-fluoro-1H-indol-3-yl)ethyl]amino}-2,3-dihydro-1H-inden-5- |
| | yl)-N-hydroxyacrylamide |
| 620 | (2E)-3-(1-{[2-(6-chloro-1H-indol-3-yl)ethyl]amino}-2,3-dihydro-1H-inden-5- |
| | yl)-N-hydroxyacrylamide |
| 621 | (2E)-N-hydroxy-3-(1-{[2-(6-methyl-1H-indol-3-yl)ethyl]amino}-2,3-dihydro- |
| | 1H-inden-5-yl)acrylamide |
| 622 | (2E)-N-hydroxy-3-(1-{[2-(1-methyl-1H-indol-3-yl)ethyl]amino}-2,3-dihydro- |
| | 1H-inden-5-yl)acrylamide |
| 623 | 4-hydroxy-N-{5-[(1E)-3-(hydroxyamino)-3-oxoprop-1-en-1-yl]-2,3-dihydro- |
| | 1H-inden-1-yl}-N-[2-(1H-indol-3-yl)ethyl]benzamide |
| 624 | N-[2-(6-fluoro-1H-indol-3-yl)ethyl]-4-hydroxy-N-{5-[(1E)-3-(hydroxyamino)- |
| | 3-oxoprop-1-en-1-yl]-2,3-dihydro-1H-inden-1-yl}benzamide |
| 625 | N-[2-(6-chloro-1H-indol-3-yl)ethyl]-4-hydroxy-N-{5-[(1E)-3-(hydroxyamino)- |
| | 3-oxoprop-1-en-1-yl]-2,3-dihydro-1H-inden-1-yl}benzamide |
| 626 | 4-hydroxy-N-{5-[(1E)-3-(hydroxyamino)-3-oxoprop-1-en-1-yl]-2,3-dihydro- |
| | 1H-inden-1-yl}-N-[2-(6-methyl-1H-indol-3-yl)ethyl]benzamide |
| 627 | 4-hydroxy-N-{5-[(1E)-3-(hydroxyamino)-3-oxoprop-1-en-1-yl]-2,3-dihydro- |
| | 1H-inden-1-yl}-N-[2-(6-methoxy-1H-indol-3-yl)ethyl]benzamide |
| 628 | 4-hydroxy-N-{5-[(1E)-3-(hydroxyamino)-3-oxoprop-1-en-1-yl]-2,3-dihydro- |
| | 1H-inden-1-yl}-N-[2-(1-methyl-1H-indol-3-yl)ethyl]benzamide |
| 629 | (2E)-N-hydroxy-3-{1-[[2-(1-methyl-1H-indol-3-yl)ethyl](1H- |
| | pyrazol-4-ylmethyl)amino]-2,3-dihydro-1H-inden-5-yl}acrylamide |
| 630 | (2E)-3-{1-[[2-(6-fluoro-1H-indol-3-yl)ethyl](1H-pyrazol- |
| | 4-ylmethyl)amino]-2,3-dihydro-1H-inden-5-yl}-N-hydroxyacrylamide |
| 631 | (2E)-3-{1-[[2-(6-chloro-1H-indol-3-yl)ethyl](1H-pyrazol- |
| | 4-ylmethyl)amino]-2,3-dihydro-1H-inden-5-yl}-N-hydroxyacrylamide |
| 632 | (2E)-N-hydroxy-3-{1-[[2-(6-methyl-1H-indol-3-yl)ethyl](1H- |
| | pyrazol-4-ylmethyl)amino]-2,3-dihydro-1H-inden-5-yl}acrylamide |
| 633 | (2E)-N-hydroxy-3-{1-[[2-(6-methoxy-1H-indol-3-yl)ethyl](1H-pyrazol-4- |
| | ylmethyl)amino]-2,3-dihydro-1H-inden-5-yl}acrylamide |
| 634 | (2E)-3-(1-{[2-(6-fluoro-1H-indol-3-yl)ethyl][(3- |
| | hydroxyphenyl)sulfonyl]amino}-2,3-dihydro-1H-inden-5-yl)-N- |
| | hydroxyacrylamide |
| 635 | (2E)-3-(1-{[2-(6-chloro-1H-indol-3-yl)ethyl][(3- |
| | hydroxyphenyl)sulfonyl]amino}-2,3-dihydro-1H-inden-5-yl)-N- |
| | hydroxyacrylamide |
| 636 | (2E)-N-hydroxy-3-(1-{[(3-hydroxyphenyl)sulfonyl][2-(6-methyl-1H-indol-3- |
| | yl)ethyl]amino}-2,3-dihydro-1H-inden-5-yl)acrylamide |
| 637 | (2E)-N-hydroxy-3-(1-{[(3-hydroxyphenyl)sulfonyl][2-(6-methoxy-1H-indol-3- |
| | yl)ethyl]amino}-2,3-dihydro-1H-inden-5-yl)acrylamide |
| 638 | (2E)-N-hydroxy-3-(1-{[(3-hydroxyphenyl)sulfonyl][2-(1-methyl-1H-indol-3- |
| | yl)ethyl]amino}-2,3-dihydro-1H-inden-5-yl)acrylamide |
| 639 | (2E)-N-hydroxy-3-(1-{[(3-hydroxyphenyl)sulfonyl][2-(1H-indol-3- |
| | yl)ethyl]amino}-2,3-dihydro-1H-inden-5-yl)acrylamide |
| 640 | (2E)-N-hydroxy-3-{1-[[2-(1H-indol-3-yl)ethyl](1H-pyrrol-3-ylmethyl)amino]- |
| | 2,3-dihydro-1H-inden-5-yl}acrylamide |
| 641 | (2E)-3-{1-[[2-(6-fluoro-1H-indol-3-yl)ethyl](1H-pyrrol-3-ylmethyl)amino]-2,3- |
| | dihydro-1H-inden-5-yl}-N-hydroxyacrylamide |
| 642 | (2E)-3-{1-[[2-(6-chloro-1H-indol-3-yl)ethyl](1H-pyrrol-3-ylmethyl)amino]- |
| | 2,3-dihydro-1H-inden-5-yl}-N-hydroxyacrylamide |
| 643 | (2E)-N-hydroxy-3-{1-[[2-(6-methyl-1H-indol-3-yl)ethyl](1H-pyrrol-3- |
| | ylmethyl)amino]-2,3-dihydro-1H-inden-5-yl}acrylamide |
| 644 | (2E)-N-hydroxy-3-{1-[[2-(1-methyl-1H-indol-3-yl)ethyl](1H-pyrrol-3- |
| | ylmethyl)amino]-2,3-dihydro-1H-inden-5-yl}acrylamide |
| 645 | (2E)-N-hydroxy-3-(1-{[2-(1H-indol-3-yl)ethyl][(2-methyl-1H-pyrrol-3- |
| | yl)methyl]amino}-2,3-dihydro-1H-inden-5-yl)acrylamide |
| 646 | (2E)-3-(1-{[2-(6-fluoro-1H-indol-3-yl)ethyl][(2-methyl-1H-pyrrol-3- |
| | yl)methyl]amino}-2,3-dihydro-1H-inden-5-yl)-N-hydroxyacrylamide |
| 647 | (2E)-3-(1-{[2-(6-chloro-1H-indol-3-yl)ethyl][(2-methyl-1H-pyrrol-3- |
| | yl)methyl]amino}-2,3-dihydro-1H-inden-5-yl)-N-hydroxyacrylamide |
| 648 | (2E)-N-hydroxy-3-(1-{[2-(6-methyl-1H-indol-3-yl)ethyl][(2-methyl-1H-pyrrol- |
| | 3-yl)methyl]amino}-2,3-dihydro-1H-inden-5-yl)acrylamide |
| 649 | (2E)-N-hydroxy-3-(1-{[2-(1-methyl-1H-indol-3-yl)ethyl][(2-methyl-1H-pyrrol- |
| | 3-yl)methyl]amino}-2,3-dihydro-1H-inden-5-yl)acrylamide |
| 650 | (2E)-3-(1-{[(4-ethyl-1H-pyrrol-3-yl)methyl][2-(1H-indol-3-yl)ethyl]amino}- |
| | 2,3-dihydro-1H-inden-5-yl)-N-hydroxyacrylamide |
| 651 | (2E)-3-(1-{[(4-ethyl-1H-pyrrol-3-yl)methyl][2-(6-fluoro- |
| | 1H-indol-3-yl)ethyl]amino}-2,3-dihydro-1H-inden-5-yl)-N- |
| | hydroxyacrylamide |
| 652 | (2E)-3-(1-{[2-(6-chloro-1H-indol-3-yl)ethyl][(4-ethyl-1H-pyrrol-3- |
| | yl)methyl]amino}-2,3-dihydro-1H-inden-5-yl)-N-hydroxyacrylamide |
| 653 | (2E)-3-(1-{[(4-ethyl-1H-pyrrol-3-yl)methyl][2-(6-methyl-1H-indol-3- |
| | yl)ethyl]amino}-2,3-dihydro-1H-inden-5-yl)-N-hydroxyacrylamide |
| 654 | (2E)-3-(1-{[(4-ethyl-1H-pyrrol-3-yl)methyl][2-(1-methyl-1H-indol-3- |
| | yl)ethyl]amino}-2,3-dihydro-1H-inden-5-yl)-N-hydroxyacrylamide |
| 655 | (2E)-3-(1-{[(5-ethyl-1H-pyrrol-3-yl)methyl][2-(1H-indol-3-yl)ethyl]amino}- |
| | 2,3-dihydro-1H-inden-5-yl)-N-hydroxyacrylamide |
| 656 | (2E)-3-(1-{[(5-ethyl-1H-pyrrol-3-yl)methyl][2-(6-fluoro- |
| | 1H-indol-3-yl)ethyl]amino}-2,3-dihydro-1H-inden-5-yl)-N- |
| | hydroxyacrylamide |
| 657 | (2E)-3-(1-{[2-(6-chloro-1H-indol-3-yl)ethyl][(5-ethyl-1H-pyrrol-3- |
| | yl)methyl]amino}-2,3-dihydro-1H-inden-5-yl)-N-hydroxyacrylamide |
| 658 | (2E)-3-(1-{[(5-ethyl-1H-pyrrol-3-yl)methyl][2-(6-methyl-1H-indol-3- |
| | yl)ethyl]amino}-2,3-dihydro-1H-inden-5-yl)-N-hydroxyacrylamide |
| 659 | (2E)-3-(1-{[(5-ethyl-1H-pyrrol-3-yl)methyl][2-(1-methyl-1H-indol-3- |
| | yl)ethyl]amino}-2,3-dihydro-1H-inden-5-yl)-N-hydroxyacrylamide |
| 660 | (2E)-3-(1-{[(2,4-dimethyl-1H-pyrrol-3-yl)methyl][2-(1H-indol-3- |
| | yl)ethyl]amino}-2,3-dihydro-1H-inden-5-yl)-N-hydroxyacrylamide |
| 661 | (2E)-3-(1-{[(2,4-dimethyl-1H-pyrrol-3-yl)methyl][2-(6-fluoro- |
| | 1H-indol-3-yl)ethyl]amino}-2,3-dihydro-1H-inden-5-yl)-N- |
| | hydroxyacrylamide |
| 662 | (2E)-3-(1-{[2-(6-chloro-1H-indol-3-yl)ethyl][(2,4-dimethyl- |
| | 1H-pyrrol-3-yl)methyl]amino}-2,3-dihydro-1H-inden-5-yl)- |
| | N-hydroxyacrylamide |
| 663 | (2E)-3-(1-{[(2,4-dimethyl-1H-pyrrol-3-yl)methyl][2-(6-methyl-1H-indol-3- |
| | yl)ethyl]amino}-2,3-dihydro-1H-inden-5-yl)-N-hydroxyacrylamide |
| 664 | (2E)-3-(1-{[(2,4-dimethyl-1H-pyrrol-3-yl)methyl][2-(1-methyl-1H-indol-3- |
| | yl)ethyl]amino}-2,3-dihydro-1H-inden-5-yl)-N-hydroxyacrylamide |
| 665 | (2E)-3-(1-{[(2,5-dimethyl-1H-pyrrol-3-yl)methyl][2-(1H-indol-3- |
| | yl)ethyl]amino}-2,3-dihydro-1H-inden-5-yl)-N-hydroxyacrylamide |
| 666 | (2E)-3-(1-{[(2,5-dimethyl-1H-pyrrol-3-yl)methyl][2-(6-fluoro- |
| | 1H-indol-3-yl)ethyl]amino}-2,3-dihydro-1H-inden-5-yl)-N- |
| | hydroxyacrylamide |
| 667 | (2E)-3-(1-{[2-(6-chloro-1H-indol-3-yl)ethyl][(2,5-dimethyl- |
| | 1H-pyrrol-3-yl)methyl]amino}-2,3-dihydro-1H-inden-5-yl)- |
| | N-hydroxyacrylamide |
| 668 | (2E)-3-(1-{[(2,5-dimethyl-1H-pyrrol-3-yl)methyl][2-(6-methyl-1H-indol-3- |
| | yl)ethyl]amino}-2,3-dihydro-1H-inden-5-yl)-N-hydroxyacrylamide |
| 669 | (2E)-3-(1-{[(2,5-dimethyl-1H-pyrrol-3-yl)methyl][2-(1-methyl-1H-indol-3- |
| | yl)ethyl]amino}-2,3-dihydro-1H-inden-5-yl)-N-hydroxyacrylamide |
| 670 | (2E)-3-(1-{[(5-chloro-1H-pyrrol-2-yl)methyl][2-(1H-indol- |
| | 3-yl)ethyl]amino}-2,3-dihydro-1H-inden-5-yl)-N-hydroxyacrylamide |
| 671 | (2E)-3-(1-{[(5-chloro-1H-pyrrol-2-yl)methyl][2-(6-fluoro- |
| | 1H-indol-3-yl)ethyl]amino}-2,3-dihydro-1H-inden-5-yl)-N- |
| | hydroxyacrylamide |
| 672 | (2E)-3-(1-{[2-(6-chloro-1H-indol-3-yl)ethyl][(5-chloro-1H- |
| | pyrrol-2-yl)methyl]amino}-2,3-dihydro-1H-inden-5-yl)-N- |
| | hydroxyacrylamide |
| 673 | (2E)-3-(1-{[(5-chloro-1H-pyrrol-2-yl)methyl][2-(6-methyl- |
| | 1H-indol-3-yl)ethyl]amino}-2,3-dihydro-1H-inden-5-yl)-N- |
| | hydroxyacrylamide |
| 674 | (2E)-3-(1-{[(5-chloro-1H-pyrrol-2-yl)methyl][2-(6-methoxy- |
| | 1H-indol-3-yl)ethyl]amino}-2,3-dihydro-1H-inden-5-yl)-N- |
| | hydroxyacrylamide |
| 675 | (2E)-3-(1-{[(5-chloro-1H-pyrrol-2-yl)methyl][2-(1-methyl- |
| | 1H-indol-3-yl)ethyl]amino}-2,3-dihydro-1H-inden-5-yl)-N- |
| | hydroxyacrylamide |
| 676 | (2E)-3-(1-{[(5-fluoro-1H-pyrrol-2-yl)methyl][2-(1H-indol- |
| | 3-yl)ethyl]amino}-2,3-dihydro-1H-inden-5-yl)-N-hydroxyacrylamide |
| 677 | (2E)-3-(1-{[2-(6-fluoro-1H-indol-3-yl)ethyl][(5-fluoro-1H- |
| | pyrrol-2-yl)methyl]amino}-2,3-dihydro-1H-inden-5-yl)-N- |
| | hydroxyacrylamide |
| 678 | (2E)-3-(1-{[2-(6-chloro-1H-indol-3-yl)ethyl][(5-fluoro-1H- |
| | pyrrol-2-yl)methyl]amino}-2,3-dihydro-1H-inden-5-yl)-N- |
| | hydroxyacrylamide |
| 679 | (2E)-3-(1-{[(5-fluoro-1H-pyrrol-2-yl)methyl][2-(6-methyl- |
| | 1H-indol-3-yl)ethyl]amino}-2,3-dihydro-1H-inden-5-yl)-N- |
| | hydroxyacrylamide |
| 680 | (2E)-3-(1-{[(5-fluoro-1H-pyrrol-2-yl)methyl][2-(6-methoxy- |
| | 1H-indol-3-yl)ethyl]amino}-2,3-dihydro-1H-inden-5-yl)-N- |
| | hydroxyacrylamide |
| 681 | (2E)-3-(1-{[(5-fluoro-1H-pyrrol-2-yl)methyl][2-(1-methyl- |
| | 1H-indol-3-yl)ethyl]amino}-2,3-dihydro-1H-inden-5-yl)-N- |
| | hydroxyacrylamide |
| 682 | (2E)-N-hydroxy-3-(1-{[2-(1H-indol-3-yl)ethyl][(5-methyl- |
| | 1H-pyrrol-2-yl)methyl]amino}-2,3-dihydro-1H-inden- |
| | 5-yl)acrylamide |
| 683 | (2E)-3-(1-{[2-(6-fluoro-1H-indol-3-yl)ethyl][(5-methyl-1H- |
| | pyrrol-2-yl)methyl]amino}-2,3-dihydro-1H-inden-5-yl)-N- |
| | hydroxyacrylamide |
| 684 | (2E)-3-(1-{[2-(6-chloro-1H-indol-3-yl)ethyl][(5-methyl-1H- |
| | pyrrol-2-yl)methyl]amino}-2,3-dihydro-1H-inden-5-yl)-N- |
| | hydroxyacrylamide |
| 685 | (2E)-N-hydroxy-3-(1-{[2-(6-methyl-1H-indol-3-yl)ethyl][(5- |
| | methyl-1H-pyrrol-2-yl)methyl]amino}-2,3-dihydro-1H-inden- |
| | 5-yl)acrylamide |
| 686 | (2E)-N-hydroxy-3-(1-{[2-(6-methoxy-1H-indol-3-yl)ethyl][(5- |
| | methyl-1H-pyrrol-2-yl)methyl]amino}-2,3-dihydro-1H-inden- |
| | 5-yl)acrylamide |
| 687 | (2E)-N-hydroxy-3-(1-{[2-(1-methyl-1H-indol-3-yl)ethyl][(5- |
| | methyl-1H-pyrrol-2-yl)methyl]amino}-2,3-dihydro-1H-inden- |
| | 5-yl)acrylamide |
| 688 | (2E)-3-(1-{[(3-chloro-1H-pyrrol-2-yl)methyl][2-(1H-indol- |
| | 3-yl)ethyl]amino}-2,3-dihydro-1H-inden-5-yl)-N-hydroxyacrylamide |
| 689 | (2E)-3-(1-{[(3-chloro-1H-pyrrol-2-yl)methyl][2-(6-fluoro- |
| | 1H-indol-3-yl)ethyl]amino}-2,3-dihydro-1H-inden-5-yl)-N- |
| | hydroxyacrylamide |
| 690 | (2E)-3-(1-{[2-(6-chloro-1H-indol-3-yl)ethyl][(3-chloro-1H- |
| | pyrrol-2-yl)methyl]amino}-2,3-dihydro-1H-inden-5-yl)-N- |
| | hydroxyacrylamide |
| 691 | (2E)-3-(1-{[(3-chloro-1H-pyrrol-2-yl)methyl][2-(6-methyl- |
| | 1H-indol-3-yl)ethyl]amino}-2,3-dihydro-1H-inden-5-yl)-N- |
| | hydroxyacrylamide |
| 692 | (2E)-3-(1-{[(3-chloro-1H-pyrrol-2-yl)methyl][2-(6-methoxy- |
| | 1H-indol-3-yl)ethyl]amino}-2,3-dihydro-1H-inden-5-yl)-N- |
| | hydroxyacrylamide |
| 693 | (2E)-3-(1-{[(3-chloro-1H-pyrrol-2-yl)methyl][2-(1-methyl- |
| | 1H-indol-3-yl)ethyl]amino}-2,3-dihydro-1H-inden-5-yl)-N- |
| | hydroxyacrylamide |
| 694 | (2E)-N-hydroxy-3-(1-{[2-(1H-indol-3-yl)ethyl][(3-methyl- |
| | 1H-pyrrol-2-yl)methyl]amino}-2,3-dihydro-1H-inden- |
| | 5-yl)acrylamide |
| 695 | (2E)-3-(1-{[2-(6-fluoro-1H-indol-3-yl)ethyl][(3-methyl-1H- |
| | pyrrol-2-yl)methyl]amino}-2,3-dihydro-1H-inden-5-yl)-N- |
| | hydroxyacrylamide |
| 696 | (2E)-3-(1-{[2-(6-chloro-1H-indol-3-yl)ethyl][(3-methyl-1H- |
| | pyrrol-2-yl)methyl]amino}-2,3-dihydro-1H-inden-5-yl)-N- |
| | hydroxyacrylamide |
| 697 | (2E)-N-hydroxy-3-(1-{[2-(6-methyl-1H-indol-3-yl)ethyl][(3- |
| | methyl-1H-pyrrol-2-yl)methyl]amino}-2,3-dihydro-1H-inden- |
| | 5-yl)acrylamide |
| 698 | (2E)-N-hydroxy-3-(1-{[2-(6-methoxy-1H-indol-3-yl)ethyl][(3- |
| | methyl-1H-pyrrol-2-yl)methyl]amino}-2,3-dihydro-1H-inden- |
| | 5-yl)acrylamide |
| 699 | (2E)-N-hydroxy-3-(1-{[2-(1-methyl-1H-indol-3-yl)ethyl][(3- |
| | methyl-1H-pyrrol-2-yl)methyl]amino}-2,3-dihydro-1H-inden- |
| | 5-yl)acrylamide |
| 700 | (2E)-3-(1-{[(4-chloro-1H-pyrrol-2-yl)methyl][2-(1H-indol- |
| | 3-yl)ethyl]amino}-2,3-dihydro-1H-inden-5-yl)-N-hydroxyacrylamide |
| 701 | (2E)-3-(1-{[(4-chloro-1H-pyrrol-2-yl)methyl][2-(6-fluoro- |
| | 1H-indol-3-yl)ethyl]amino}-2,3-dihydro-1H-inden-5-yl)-N- |
| | hydroxyacrylamide |
| 702 | (2E)-3-(1-{[2-(6-chloro-1H-indol-3-yl)ethyl][(4-chloro-1H- |
| | pyrrol-2-yl)methyl]amino}-2,3-dihydro-1H-inden-5-yl)-N- |
| | hydroxyacrylamide |
| 703 | (2E)-3-(1-{[(4-chloro-1H-pyrrol-2-yl)methyl][2-(6-methyl- |
| | 1H-indol-3-yl)ethyl]amino}-2,3-dihydro-1H-inden-5-yl)-N- |
| | hydroxyacrylamide |
| 704 | (2E)-3-(1-{[(4-chloro-1H-pyrrol-2-yl)methyl][2-(6-methoxy- |
| | 1H-indol-3-yl)ethyl]amino}-2,3-dihydro-1H-inden-5-yl)-N- |
| | hydroxyacrylamide |
| 705 | (2E)-3-(1-{[(4-chloro-1H-pyrrol-2-yl)methyl][2-(1-methyl- |
| | 1H-indol-3-yl)ethyl]amino}-2,3-dihydro-1H-inden-5-yl)-N- |
| | hydroxyacrylamide |
| 706 | (2E)-N-hydroxy-3-(1-{[2-(1H-indol-3-yl)ethyl][(4-methyl- |
| | 1H-pyrrol-2-yl)methyl]amino}-2,3-dihydro-1H-inden- |
| | 5-yl)acrylamide |
| 707 | (2E)-3-(1-{[2-(6-fluoro-1H-indol-3-yl)ethyl][(4-methyl-1H- |
| | pyrrol-2-yl)methyl]amino}-2,3-dihydro-1H-inden-5-yl)-N- |
| | hydroxyacrylamide |
| 708 | (2E)-3-(1-{[2-(6-chloro-1H-indol-3-yl)ethyl][(4-methyl-1H- |
| | pyrrol-2-yl)methyl]amino}-2,3-dihydro-1H-inden-5-yl)-N- |
| | hydroxyacrylamide |
| 709 | (2E)-N-hydroxy-3-(1-{[2-(6-methyl-1H-indol-3-yl)ethyl][(4- |
| | methyl-1H-pyrrol-2-yl)methyl]amino}-2,3-dihydro-1H-inden- |
| | 5-yl)acrylamide |
| 710 | (2E)-N-hydroxy-3-(1-{[2-(6-methoxy-1H-indol-3-yl)ethyl][(4- |
| | methyl-1H-pyrrol-2-yl)methyl]amino}-2,3-dihydro-1H-inden- |
| | 5-yl)acrylamide |
| 711 | (2E)-N-hydroxy-3-(1-{[2-(1-methyl-1H-indol-3-yl)ethyl][(4- |
| | methyl-1H-pyrrol-2-yl)methyl]amino}-2,3-dihydro-1H-inden- |
| | 5-yl)acrylamide |
| 712 | (2E)-3-(1-{[(3,4-dimethyl-1H-pyrrol-2-yl)methyl][2-(1H-indol-3- |
| | yl)ethyl]amino}-2,3-dihydro-1H-inden-5-yl)-N-hydroxyacrylamide |
| 713 | (2E)-3-(1-{[(3,4-dimethyl-1H-pyrrol-2-yl)methyl][2-(6-fluoro- |
| | 1H-indol-3-yl)ethyl]amino}-2,3-dihydro-1H-inden-5-yl)-N- |
| | hydroxyacrylamide |
| 714 | (2E)-3-(1-{[2-(6-chloro-1H-indol-3-yl)ethyl][(3,4-dimethyl- |
| | 1H-pyrrol-2-yl)methyl]amino}-2,3-dihydro-1H-inden-5-yl)- |
| | N-hydroxyacrylamide |
| 715 | (2E)-3-(1-{[(3,4-dimethyl-1H-pyrrol-2-yl)methyl][2-(6-methyl-1H-indol-3- |
| | yl)ethyl]amino}-2,3-dihydro-1H-inden-5-yl)-N-hydroxyacrylamide |
| 716 | (2E)-3-(1-{[(3,4-dimethyl-1H-pyrrol-2-yl)methyl][2-(6-methoxy-1H-indol-3- |
| | yl)ethyl]amino}-2,3-dihydro-1H-inden-5-yl)-N-hydroxyacrylamide |
| 717 | (2E)-3-(1-{[(3,4-dimethyl-1H-pyrrol-2-yl)methyl][2-(1-methyl-1H-indol-3- |
| | yl)ethyl]amino}-2,3-dihydro-1H-inden-5-yl)-N-hydroxyacrylamide |
| 718 | (2E)-3-(1-{[(4-chloro-3-methyl-1H-pyrrol-2-yl)methyl][2- |
| | (1H-indol-3-yl)ethyl]amino}-2,3-dihydro-1H-inden-5-yl)- |
| | N-hydroxyacrylamide |
| 719 | (2E)-3-(1-{[(4-chloro-3-methyl-1H-pyrrol-2-yl)methyl][2- |
| | (6-fluoro-1H-indol-3-yl)ethyl]amino}-2,3-dihydro-1H-inden- |
| | 5-yl)-N-hydroxyacrylamide |
| 720 | (2E)-3-(1-{[2-(6-chloro-1H-indol-3-yl)ethyl][(4-chloro-3- |
| | methyl-1H-pyrrol-2-yl)methyl]amino}-2,3-dihydro-1H-inden- |
| | 5-yl)-N-hydroxyacrylamide |
| 721 | (2E)-3-(1-{[(4-chloro-3-methyl-1H-pyrrol-2-yl)methyl][2- |
| | (6-methyl-1H-indol-3-yl)ethyl]amino}-2,3-dihydro-1H-inden- |
| | 5-yl)-N-hydroxyacrylamide |
| 722 | (2E)-3-(1-{[(4-chloro-3-methyl-1H-pyrrol-2-yl)methyl][2- |
| | (6-methoxy-1H-indol-3-yl)ethyl]amino}-2,3-dihydro-1H-inden-5-yl)-N- |
| | hydroxyacrylamide |
| 723 | (2E)-3-(1-{[(4-chloro-3-methyl-1H-pyrrol-2-yl)methyl][2- |
| | (1-methyl-1H-indol-3-yl)ethyl]amino}-2,3-dihydro-1H-inden- |
| | 5-yl)-N-hydroxyacrylamide |
| 724 | (2E)-3-(1-{[(3,4-dichloro-1H-pyrrol-2-yl)methyl][2-(1H-indol- |
| | 3-yl)ethyl]amino}-2,3-dihydro-1H-inden-5-yl)-N-hydroxyacrylamide |
| 725 | (2E)-3-(1-{[(3,4-dichloro-1H-pyrrol-2-yl)methyl][2-(6-fluoro- |
| | 1H-indol-3-yl)ethyl]amino}-2,3-dihydro-1H-inden-5-yl)-N- |
| | hydroxyacrylamide |
| 726 | (2E)-3-(1-{[2-(6-chloro-1H-indol-3-yl)ethyl][(3,4-dichloro- |
| | 1H-pyrrol-2-yl)methyl]amino}-2,3-dihydro-1H-inden-5-yl)- |
| | N-hydroxyacrylamide |
| 727 | (2E)-3-(1-{[(3,4-dichloro-1H-pyrrol-2-yl)methyl][2-(6-methyl-1H-indol-3- |
| | yl)ethyl]amino}-2,3-dihydro-1H-inden-5-yl)-N-hydroxyacrylamide |
| 728 | (2E)-3-(1-{[(3,4-dichloro-1H-pyrrol-2-yl)methyl][2-(6-methoxy-1H-indol-3- |
| | yl)ethyl]amino}-2,3-dihydro-1H-inden-5-yl)-N-hydroxyacrylamide |
| 729 | (2E)-3-(1-{[(3,4-dichloro-1H-pyrrol-2-yl)methyl][2-(1-methyl-1H-indol-3- |
| | yl)ethyl]amino}-2,3-dihydro-1H-inden-5-yl)-N-hydroxyacrylamide |
| 730 | (2E)-3-(1-{[(5-chloro-3,4-dimethyl-1H-pyrrol-2-yl)methyl][2- |
| | (1H-indol-3-yl)ethyl]amino}-2,3-dihydro-1H-inden-5-yl)- |
| | N-hydroxyacrylamide |
| 731 | (2E)-3-(1-{[(5-chloro-3,4-dimethyl-1H-pyrrol-2-yl)methyl][2- |
| | (6-fluoro-1H-indol-3-yl)ethyl]amino}-2,3-dihydro-1H-inden- |
| | 5-yl)-N-hydroxyacrylamide |
| 732 | (2E)-3-(1-{[(5-chloro-3,4-dimethyl-1H-pyrrol-2-yl)methyl][2- |
| | (6-chloro-1H-indol-3-yl)ethyl]amino}-2,3-dihydro-1H-inden- |
| | 5-yl)-N-hydroxyacrylamide |
| 733 | (2E)-3-(1-{[(5-chloro-3,4-dimethyl-1H-pyrrol-2-yl)methyl][2- |
| | (6-methyl-1H-indol-3-yl)ethyl]amino}-2,3-dihydro-1H-inden- |
| | 5-yl)-N-hydroxyacrylamide |
| 734 | (2E)-3-(1-{[(5-chloro-3,4-dimethyl-1H-pyrrol-2-yl)methyl][2- |
| | (6-methoxy-1H-indol-3-yl)ethyl]amino}-2,3-dihydro-1H-inden-5-yl)-N- |
| | hydroxyacrylamide |
| 735 | (2E)-3-(1-{[(5-chloro-3,4-dimethyl-1H-pyrrol-2-yl)methyl][2- |
| | (1-methyl-1H-indol-3-yl)ethyl]amino}-2,3-dihydro-1H-inden- |
| | 5-yl)-N-hydroxyacrylamide |
| 736 | (2E)-N-hydroxy-3-(1-{[2-(1H-indol-3-yl)ethyl][(3,4,5-trimethyl-1H-pyrrol-2- |
| | yl)methyl]amino}-2,3-dihydro-1H-inden-5-yl)acrylamide |
| 737 | (2E)-3-(1-{[2-(6-fluoro-1H-indol-3-yl)ethyl][(3,4,5-trimethyl- |
| | 1H-pyrrol-2-yl)methyl]amino}-2,3-dihydro-1H-inden-5-yl)- |
| | N-hydroxyacrylamide |
| 738 | (2E)-3-(1-{[2-(6-chloro-1H-indol-3-yl)ethyl][(3,4,5-trimethyl- |
| | 1H-pyrrol-2-yl)methyl]amino}-2,3-dihydro-1H-inden-5-yl)- |
| | N-hydroxyacrylamide |
| 739 | (2E)-N-hydroxy-3-(1-{[2-(6-methyl-1H-indol-3-yl)ethyl][(3,4,5-trimethyl-1H- |
| | pyrrol-2-yl)methyl]amino}-2,3-dihydro-1H-inden-5-yl)acrylamide |
| 740 | (2E)-N-hydroxy-3-(1-{[2-(6-methoxy-1H-indol-3-yl)ethyl][(3,4,5-trimethyl- |
| | 1H-pyrrol-2-yl)methyl]amino}-2,3-dihydro-1H-inden-5-yl)acrylamide |
| 741 | (2E)-N-hydroxy-3-(1-{[2-(1-methyl-1H-indol-3-yl)ethyl][(3,4,5-trimethyl-1H- |
| | pyrrol-2-yl)methyl]amino}-2,3-dihydro-1H-inden-5-yl)acrylamide |
| 742 | (2E)-3-(1-{[(4-fluoro-3,5-dimethyl-1H-pyrrol-2-yl)methyl][2- |
| | (1H-indol-3-yl)ethyl]amino}-2,3-dihydro-1H-inden-5-yl)- |
| | N-hydroxyacrylamide |
| 743 | (2E)-3-(1-{[(4-fluoro-3,5-dimethyl-1H-pyrrol-2-yl)methyl][2- |
| | (6-fluoro-1H-indol-3-yl)ethyl]amino}-2,3-dihydro-1H-inden- |
| | 5-yl)-N-hydroxyacrylamide |
| 744 | (2E)-3-(1-{[2-(6-chloro-1H-indol-3-yl)ethyl][(4-fluoro-3,5- |
| | dimethyl-1H-pyrrol-2-yl)methyl]amino}-2,3-dihydro-1H-inden-5-yl)-N- |
| | hydroxyacrylamide |
| 745 | (2E)-3-(1-{[(4-fluoro-3,5-dimethyl-1H-pyrrol-2-yl)methyl][2- |
| | (6-methyl-1H-indol-3-yl)ethyl]amino}-2,3-dihydro-1H-inden- |
| | 5-yl)-N-hydroxyacrylamide |
| 746 | (2E)-3-(1-{[(4-fluoro-3,5-dimethyl-1H-pyrrol-2-yl)methyl][2- |
| | (6-methoxy-1H-indol-3-yl)ethyl]amino}-2,3-dihydro-1H-inden-5-yl)-N- |
| | hydroxyacrylamide |
| 747 | (2E)-3-(1-{[(4-fluoro-3,5-dimethyl-1H-pyrrol-2-yl)methyl][2- |
| | (1-methyl-1H-indol-3-yl)ethyl]amino}-2,3-dihydro-1H-inden- |
| | 5-yl)-N-hydroxyacrylamide |
| 748 | (2E)-3-(1-{[(4,5-dimethyl-1H-pyrrol-2-yl)methyl][2-(1H-indol-3- |
| | yl)ethyl]amino}-2,3-dihydro-1H-inden-5-yl)-N-hydroxyacrylamide |
| 749 | (2E)-3-(1-{[(4,5-dimethyl-1H-pyrrol-2-yl)methyl][2-(6-fluoro- |
| | 1H-indol-3-yl)ethyl]amino}-2,3-dihydro-1H-inden-5-yl)-N- |
| | hydroxyacrylamide |
| 750 | (2E)-3-(1-{[2-(6-chloro-1H-indol-3-yl)ethyl][(4,5-dimethyl- |
| | 1H-pyrrol-2-yl)methyl]amino}-2,3-dihydro-1H-inden-5-yl)- |
| | N-hydroxyacrylamide |
| 751 | (2E)-3-(1-{[(4,5-dimethyl-1H-pyrrol-2-yl)methyl][2-(6-methyl-1H-indol-3- |
| | yl)ethyl]amino}-2,3-dihydro-1H-inden-5-yl)-N-hydroxyacrylamide |
| 752 | (2E)-3-(1-{[(4,5-dimethyl-1H-pyrrol-2-yl)methyl][2-(6-methoxy-1H-indol-3- |
| | yl)ethyl]amino}-2,3-dihydro-1H-inden-5-yl)-N-hydroxyacrylamide |
| 753 | (2E)-3-(1-{[(4,5-dimethyl-1H-pyrrol-2-yl)methyl][2-(1-methyl-1H-indol-3- |
| | yl)ethyl]amino}-2,3-dihydro-1H-inden-5-yl)-N-hydroxyacrylamide |
| 754 | (2E)-3-(1-{[(4,5-dichloro-1H-pyrrol-2-yl)methyl][2-(1H-indol- |
| | 3-yl)ethyl]amino}-2,3-dihydro-1H-inden-5-yl)-N-hydroxyacrylamide |
| 755 | (2E)-3-(1-{[(4,5-dichloro-1H-pyrrol-2-yl)methyl][2-(6-fluoro- |
| | 1H-indol-3-yl)ethyl]amino}-2,3-dihydro-1H-inden-5-yl)-N- |
| | hydroxyacrylamide |
| 756 | (2E)-3-(1-{[2-(6-chloro-1H-indol-3-yl)ethyl][(4,5-dichloro- |
| | 1H-pyrrol-2-yl)methyl]amino}-2,3-dihydro-1H-inden-5-yl)- |
| | N-hydroxyacrylamide |
| 757 | (2E)-3-(1-{[(4,5-dichloro-1H-pyrrol-2-yl)methyl][2-(6-methyl-1H-indol-3- |
| | yl)ethyl]amino}-2,3-dihydro-1H-inden-5-yl)-N-hydroxyacrylamide |
| 758 | (2E)-3-(1-{[(4,5-dichloro-1H-pyrrol-2-yl)methyl][2-(6-methoxy-1H-indol-3- |
| | yl)ethyl]amino}-2,3-dihydro-1H-inden-5-yl)-N-hydroxyacrylamide |
| 759 | (2E)-3-(1-{[(4,5-dichloro-1H-pyrrol-2-yl)methyl][2-(1-methyl-1H-indol-3- |
| | yl)ethyl]amino}-2,3-dihydro-1H-inden-5-yl)-N-hydroxyacrylamide |
| 760 | (2E)-3-(1-{[(3,5-dimethyl-1H-pyrrol-2-yl)methyl][2-(1H-indol- |
| | 3-yl)ethyl]amino}-2,3-dihydro-1H-inden-5-yl)-N-hydroxyacrylamide |
| 761 | (2E)-3-(1-{[(3,5-dimethyl-1H-pyrrol-2-yl)methyl][2-(6-fluoro- |
| | 1H-indol-3-yl)ethyl]amino}-2,3-dihydro-1H-inden-5-yl)-N- |
| | hydroxyacrylamide |
| 762 | (2E)-3-(1-{[2-(6-chloro-1H-indol-3-yl)ethyl][(3,5-dimethyl- |
| | 1H-pyrrol-2-yl)methyl]amino}-2,3-dihydro-1H-inden-5-yl)- |
| | N-hydroxyacrylamide |
| 763 | (2E)-3-(1-{[(3,5-dimethyl-1H-pyrrol-2-yl)methyl][2-(6-methyl-1H-indol-3- |
| | yl)ethyl]amino}-2,3-dihydro-1H-inden-5-yl)-N-hydroxyacrylamide |
| 764 | (2E)-3-(1-{[(3,5-dimethyl-1H-pyrrol-2-yl)methyl][2-(6-methoxy-1H-indol-3- |
| | yl)ethyl]amino}-2,3-dihydro-1H-inden-5-yl)-N-hydroxyacrylamide |
| 765 | (2E)-3-(1-{[(3,5-dimethyl-1H-pyrrol-2-yl)methyl][2-(1-methyl-1H-indol-3- |
| | yl)ethyl]amino}-2,3-dihydro-1H-inden-5-yl)-N-hydroxyacrylamide |
| 766 | 4-[([2-(6-fluoro-1H-indol-3-yl)ethyl]{5-[(1E)-3-(hydroxyamino)-3-oxoprop-1- |
| | en-1-yl]-2,3-dihydro-1H-inden-1-yl}amino)sulfonyl]benzoic acid |
| 767 | 4-[([2-(6-chloro-1H-indol-3-yl)ethyl]{5-[(1E)-3-(hydroxyamino)-3-oxoprop-1- |
| | en-1-yl]-2,3-dihydro-1H-inden-1-yl}amino)sulfonyl]benzoic acid |
| 768 | 4-({{5-[(1E)-3-(hydroxyamino)-3-oxoprop-1-en-1-yl]-2,3-dihydro-1H-inden- |
| | 1-yl}[2-(6-methyl-1H-indol-3-yl)ethyl]amino}sulfonyl)benzoic acid |
| 769 | 4-({{5-[(1E)-3-(hydroxyamino)-3-oxoprop-1-en-1-yl]-2,3-dihydro-1H-inden- |
| | 1-yl}[2-(6-methoxy-1H-indol-3-yl)ethyl]amino}sulfonyl)benzoic acid |
| 770 | 4-({{5-[(1E)-3-(hydroxyamino)-3-oxoprop-1-en-1-yl]-2,3-dihydro-1H-inden- |
| | 1-yl}[2-(1-methyl-1H-indol-3-yl)ethyl]amino}sulfonyl)benzoic acid |
| 771 | 4-({{5-[(1E)-3-(hydroxyamino)-3-oxoprop-1-en-1-yl]-2,3-dihydro-1H-inden- |
| | 1-yl}[2-(1H-indol-3-yl)ethyl]amino}sulfonyl)benzoic acid |
| 772 | 4-({{5-[(1E)-3-(hydroxyamino)-3-oxoprop-1-en-1-yl]-2,3-dihydro-1H-inden- |
| | 1-yl}[2-(1H-indol-3-yl)ethyl]amino}methyl)-N-methyl-1H-pyrazole-5- |
| | carboxamide |
| 773 | 4-[([2-(6-fluoro-1H-indol-3-yl)ethyl]{5-[(1E)-3-(hydroxyamino)-3-oxoprop-1- |
| | en-1-yl]-2,3-dihydro-1H-inden-1-yl}amino)methyl]-N-methyl-1H-pyrazole-5- |
| | carboxamide |
| 774 | 4-[([2-(6-chloro-1H-indol-3-yl)ethyl]{5-[(1E)-3-(hydroxyamino)-3-oxoprop-1- |
| | en-1-yl]-2,3-dihydro-1H-inden-1-yl}amino)methyl]-N-methyl-1H-pyrazole-5- |
| | carboxamide |
| 775 | 4-({{5-[(1E)-3-(hydroxyamino)-3-oxoprop-1-en-1-yl]-2,3-dihydro-1H-inden- |
| | 1-yl}[2-(6-methyl-1H-indol-3-yl)ethyl]amino}methyl)-N-methyl-1H-pyrazole- |
| | 5-carboxamide |
| 776 | 4-({{5-[(1E)-3-(hydroxyamino)-3-oxoprop-1-en-1-yl]-2,3-dihydro-1H-inden- |
| | 1-yl}[2-(6-methoxy-1H-indol-3-yl)ethyl]amino}methyl)-N-methyl-1H- |
| | pyrazole-5-carboxamide |
| 777 | 4-({{5-[(1E)-3-(hydroxyamino)-3-oxoprop-1-en-1-yl]-2,3-dihydro-1H-inden- |
| | 1-yl}[2-(1-methyl-1H-indol-3-yl)ethyl]amino}methyl)-N-methyl-1H-pyrazole- |
| | 5-carboxamide |
| 778 | (2E)-N-hydroxy-3-[1-([2-(1H-indol-3-yl)ethyl]{[5-(morpholin- |
| | 4-ylcarbonyl)-1H-pyrazol-4-yl]methyl}amino)-2,3-dihydro- |
| | 1H-inden-5-yl]acrylamide |
| 779 | (2E)-3-[1-([2-(6-fluoro-1H-indol-3-yl)ethyl]{[5-(morpholin- |
| | 4-ylcarbonyl)-1H-pyrazol-4-yl]methyl}amino)-2,3-dihydro- |
| | 1H-inden-5-yl]-N-hydroxyacrylamide |
| 780 | (2E)-3-[1-([2-(6-chloro-1H-indol-3-yl)ethyl]{[5-(morpholin- |
| | 4-ylcarbonyl)-1H-pyrazol-4-yl]methyl}amino)-2,3-dihydro- |
| | 1H-inden-5-yl]-N-hydroxyacrylamide |
| 781 | (2E)-N-hydroxy-3-[1-([2-(6-methyl-1H-indol-3-yl)ethyl]{[5- |
| | (morpholin-4-ylcarbonyl)-1H-pyrazol-4-yl]methyl}amino)- |
| | 2,3-dihydro-1H-inden-5-yl]acrylamide |
| 782 | (2E)-N-hydroxy-3-[1-([2-(6-methoxy-1H-indol-3-yl)ethyl]{[5- |
| | (morpholin-4-ylcarbonyl)-1H-pyrazol-4-yl]methyl}amino)- |
| | 2,3-dihydro-1H-inden-5-yl]acrylamide |
| 783 | (2E)-N-hydroxy-3-[1-([2-(1-methyl-1H-indol-3-yl)ethyl]{[5- |
| | (morpholin-4-ylcarbonyl)-1H-pyrazol-4-yl]methyl}amino)- |
| | 2,3-dihydro-1H-inden-5-yl]acrylamide |
| 784 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]-1H-pyrrole-2-carboxamide |
| 785 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]-5-methyl-1H-pyrrole-2-carboxamide |
| 786 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]-1H-imidazole-2-carboxamide |
| 787 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]-2-furamide |
| 788 | N-{5-[(1E)-3-(hydroxyamino)-3-oxoprop-1-en-1-yl]-2,3-dihydro-1H-inden-1- |
| | yl}-N-[2-(1H-indol-3-yl)ethyl]thiophene-2-carboxamide |
| 789 | (2E)-3-(1-{[(4-acetyl-1H-pyrrol-2-yl)methyl][2-(1H-indol- |
| | 3-yl)ethyl]amino}-2,3-dihydro-1H-inden-5-yl)-N-hydroxyacrylamide |
| 790 | (2E)-N-hydroxy-3-(1-{[2-(6-methoxy-1H-indol-3-yl)ethyl][(2- |
| | methyl-1H-pyrrol-3-yl)methyl]amino}-2,3-dihydro-1H-inden- |
| | 5-yl)acrylamide |
| 791 | (2E)-N-hydroxy-3-{1-[[2-(6-methoxy-1H-indol-3-yl)ethyl](1H-pyrrol-3- |
| | ylmethyl)amino]-2,3-dihydro-1H-inden-5-yl}acrylamide |
| 792 | (2E)-3-(1-{[(4-ethyl-1H-pyrrol-3-yl)methyl][2-(6-methoxy- |
| | 1H-indol-3-yl)ethyl]amino}-2,3-dihydro-1H-inden-5-yl)-N- |
| | hydroxyacrylamide |
| 793 | (2E)-3-(1-{[(5-ethyl-1H-pyrrol-3-yl)methyl][2-(6-methoxy- |
| | 1H-indol-3-yl)ethyl]amino}-2,3-dihydro-1H-inden-5-yl)-N- |
| | hydroxyacrylamide |
| 794 | (2E)-3-(1-{[(2,4-dimethyl-1H-pyrrol-3-yl)methyl][2-(6-methoxy-1H-indol-3- |
| | yl)ethyl]amino}-2,3-dihydro-1H-inden-5-yl)-N-hydroxyacrylamide |
| 795 | (2E)-3-(1-{[(2,5-dimethyl-1H-pyrrol-3-yl)methyl][2-(6-methoxy-1H-indol-3- |
| | yl)ethyl]amino}-2,3-dihydro-1H-inden-5-yl)-N-hydroxyacrylamide |
| 796 | (2E)-3-(1-{[2-(6-fluoro-1H-indol-3-yl)ethyl]amino}-2,3-dihydro-1H-inden-5- |
| | yl)-N-hydroxyacrylamide |
| 797 | (2E)-3-(1-{[2-(6-fluoro-1H-indol-3-yl)ethyl]amino}-2,3-dihydro-1H-inden-5- |
| | yl)-N-hydroxyacrylamide |
| 798 | (2E)-3-(1-{[2-(6-chloro-1H-indol-3-yl)ethyl]amino}-2,3-dihydro-1H-inden-5- |
| | yl)-N-hydroxyacrylamide |
| 799 | (2E)-3-(1-{[2-(6-chloro-1H-indol-3-yl)ethyl]amino}-2,3-dihydro-1H-inden-5-yl)- |
| | N-hydroxyacrylamide |
| 800 | (2E)-N-hydroxy-3-(1-{[2-(6-methyl-1H-indol-3-yl)ethyl]amino}-2,3-dihydro- |
| | 1H-inden-5-yl)acrylamide |
| 801 | (2E)-N-hydroxy-3-(1-{[2-(6-methyl-1H-indol-3-yl)ethyl]amino}-2,3-dihydro-1H- |
| | inden-5-yl)acrylamide |
| 802 | (2E)-N-hydroxy-3-(1-{[2-(1-methyl-1H-indol-3-yl)ethyl]amino}-2,3-dihydro- |
| | 1H-inden-5-yl)acrylamide |
| 803 | (2E)-N-hydroxy-3-(1-{[2-(1-methyl-1H-indol-3-yl)ethyl]amino}-2,3-dihydro-1H- |
| | inden-5-yl)acrylamide |
| 804 | (2E)-3-(1-{ethyl[2-(6-fluoro-1H-indol-3-yl)ethyl]amino}-2,3-dihydro-1H- |
| | inden-5-yl)-N-hydroxyacrylamide |
| 805 | (2E)-3-(1-{ethyl[2-(6-fluoro-1H-indol-3-yl)ethyl]amino}-2,3-dihydro-1H- |
| | inden-5-yl)-N-hydroxyacrylamide |
| 806 | (2E)-3-(1-{ethyl[2-(6-fluoro-1H-indol-3-yl)ethyl]amino}-2,3-dihydro-1H- |
| | inden-5-yl)-N-hydroxyacrylamide |
| 807 | (2E)-N-hydroxy-3-(1-{[2-(5-methoxy-1H-indol-1-yl)ethyl]amino}-2,3- |
| | dihydro-1H-inden-5-yl)acrylamide |
| 808 | (2E)-N-hydroxy-3-(1-{[2-(5-methoxy-1H-indol-1-yl)ethyl]amino}-2,3- |
| | dihydro-1H-inden-5-yl)acrylamide |

The compounds of this invention may contain one or more asymmetric centers, depending upon the location and nature of the various substituents desired. Asymmetric carbon atoms may be present in the (R) or (S) configuration. In certain instances, asymmetry may also be present due to restricted rotation about a given bond, for example, the central bond adjoining two substituted aromatic rings of the specified compounds. Substituents on a ring may also be present in either cis or trans form, and a substituent on a double bond may be present in either =Z- or =E-form. It is intended that all such configurations (including enantiomers and diastereomers) are included within the scope of the present invention. Preferred compounds are those with the absolute configuration of the compound of this invention which produces the more desirable biological activity. Separated, pure or partially purified isomers or racemic mixtures of the compounds of this invention are also included within the scope of the present invention. The purification of said isomers and the separation of said isomeric mixtures can be accomplished by standard techniques known in the art.

For the compounds containing one or more asymmetric centers, (±), (+), or (−) is used to describe the racemic mixture, the enantiomer with the positive optical rotation, or the negative rotation, respectively. In the absence of any (+) or (−) sign before a structure or a chemical name, the compound described is a racemic mixture with the relative stereochemistry shown. The exceptions are examples 1, 7, 16, 40, 41, 42, and 128 and their corresponding chiral intermediates. The absolute stereochemistry is depicted by the structures and/or IUPAC names.

Pharmaceutically acceptable salts of these compounds are also within the scope of this invention. The term “pharmaceutically acceptable salt” refers to a relatively non-toxic, inorganic or organic salt of a compound of the present invention. For example, see S. M. Berge, et al., “Pharmaceutical Salts,” J. Pharm. Sci., 66: 1-19, 1977.

Representative salts of the compounds of this invention include the conventional non-toxic salts and the quaternary ammonium salts that are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Pro-Drugs of the Present Invention

It is anticipated that pro-drug forms of the compounds identified above will prove useful in certain circumstances, and such compounds are also intended to fall within the scope of the invention. A pro-drug, for the purpose of this invention, is a compound that is converted into its parent compound by one or more metabolic processes within a patient's body. Such conversion processes include the major drug biotransformation reactions described in Goodman and Gilman's The Pharmacological Basis of Therapeutics (Ninth Edition), editor Molinoff et al., publ. by McGraw-Hill, pages 11-13, (1996), which is hereby incorporated by reference, including, but not by way of limitation, hydrolysis in the stomach, gut or plasma.

A pro-drug compound may have advantages over its parent compound in that it may be better absorbed, better distributed, and/or it may more readily penetrate the central nervous system, be more slowly metabolized or cleared, and the like. Pro-drug forms may also have formulation advantages in terms of crystallinity or water solubility. Accordingly, a pro-drug of this invention may have a chemical structure that enhances the properties of the parent compound into which it may be metabolized. Additional examples of such enhanced properties include those described in, for example, “Pharmaceutical Dosage Form and Drug Delivery Systems” (Sixth Edition), edited by Ansel et al., publ. by Williams & Wilkins, pgs. 27-29, (1995), which is incorporated herein by reference.

Examples of pro-drugs include parent compounds identified in the Tables above that have one or more hydroxyl groups where the hydroxyl groups on these compounds are converted to ester or carbonate groups. Such esters include alkyl esters such as methyl, ethyl, propyl, isopropyl, butyl, isobutyl, pentyl and alkyl-phenyl esters, and the like. Specific examples of esters include acetate and benzoate. Examples of the carbonates of the compounds of this invention include pharmaceutically acceptable carbonates such as methyl, ethyl, propyl, isopropyl, butyl, isobutyl or pentyl carbonate. Specific examples of carbonates include O—C(═O)—CH₂CH₃ (ethyl carbonate) and O—C(═O)—CH(CH₃)₂ (isopropyl carbonate).

These ester or carbonate group(s) may be hydrolyzed at physiological pH values, may be cleaved by endogenous esterases or lipases, or otherwise may be cleaved in vivo to release the parent compound as the active material for treating hyper-proliferative disorders. (See, e.g., U.S. Pat. No. 4,942,184, U.S. Pat. No. 4,960,790, U.S. Pat. No. 5,817,840, and U.S. Pat. No. 5824701, all of which are incorporated herein by reference in their entirety, including references therein.)

Unless the context clearly indicates to the contrary, whenever the term “compounds of this invention,” “compounds of the present invention”, and the like, are used herein, they are intended to include the chemically feasible pharmaceutically acceptable salts and/or esters as well as all stereoisomeric forms of the referenced compounds.

Method of Making the Compounds of the Present Invention

In general, the compounds used in this invention may be prepared by standard techniques known in the art, by known processes analogous thereto, and/or by the processes described herein, using starting materials which are either commercially available or producible according to routine, conventional chemical methods. The particular process to be utilized in the preparation of the compounds of this invention depends upon the specific compound desired. Such factors as whether the amine is substituted or not, the selection of the specific substituents possible at various locations on the molecule, and the like, each play a role in the path to be followed in the preparation of the specific compounds of this invention. Those factors are readily recognized by one of ordinary skill in the art.

The following preparative methods are presented to aid the reader in the synthesis of the compounds of the present invention.

Abbreviations and Acronyms

When the following abbreviations and symbols are used herein, they have the following meaning:

| | |
|---|---|
| [α]_{D} | optical rotation |
| AcOH | acetic acid |
| Boc | tert-butylcarboxy |
| DIBAL | diisobutylaluminum hydride |
| DMAP | 4-dimethylaminopyridine |
| DMF | N,N-dimethylformamide |
| DIPEA | diisopropylethylamine |
| DMSO | dimethylsulfoxide |
| DPPP | bis(diphenylphosphino)propane |
| EA | elemental analysis |
| ES | electrospray |
| Et₃N | triethylamine |
| Et₂O | diethyl ether |
| EtOAc | ethyl acetate |
| GC-MS | Gas chromatography -mass spectrometry |
| h | hour |
| Hex | Hexanes |
| HPLC | high performance liquid chromatography |
| iPrOH | 2-propanol |
| LC-MS | Liquid Chromatography/Mass Spectrometry |
| Me | methyl |
| MeOH | methanol |
| min | minutes |
| NaBH(OAc)₃ | sodium triacetoxyborohydride |
| NMR | Nuclear Magnetic Resonance Spectroscopy |
| OTBDMS | tert-butyl(dimethyl)silyloxy |
| OMe | methoxy |
| Pd(OAc)₂ | palladium (II) acetate |
| PyBOP | Bromotripyrrolidinophosphonium hexafluorophosphate |
| R_{f} | TLC Retention Factor |
| RT | retention time (HPLC) |
| rt | room temperature |
| TBDMS | tert-butyldimethylsilyl |
| THF | tetrahydrofuran |
| TLC | thin layer chromatography |

### Experimental Procedures:

LC-MS Methods

Method A:

HPLC—electrospray mass spectra (HPLC ES-MS) were obtained using a Hewlett-Packard 1100 HPLC equipped with a quatemary pump, a variable wavelength detector set at 254 nm, a YMC pro C-18 column (2×23 mm, 120A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonirile with 0.018% TFA. Gradient elution from 10% B to 95% over 3.5 minutes at a flowrate of 1.0 mUmin was used with an initial hold of 0.5 minutes and a final hold at 95% B of 0.5 minutes. Total run time was 6.5 minutes.

Method B:

HPLC—electrospray mass spectra (HPLC ES-MS) were obtained using a Gilson HPLC system equipped with two Gilson 306 pumps, a Gilson 215 Autosampler, a Gilson diode array detector, a YMC Pro C-18 column (2×23mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data was also acquired as an analog channel. The eluents were A: 2% acetonitrile in water with 0.02% TFA and B: 2% water in acetonirile with 0.018% TFA. Gradient elution from 10% B to 90% over 3.5 minutes at a flowrate of 1.5 mUmin was used with an initial hold of 0.5 minutes and a final hold at 90% B of 0.5 minutes. Total run time was 4.8 minutes.

NMR Methods

Proton (¹H) nuclear magnetic resonance (NMR) spectra were measured with a Varian Mercury (300 MHz) or a Bruker Avance (500 MHz) spectrometer with either Me₄Si (δ 0.00) or residual protonated solvent (CHCl₃ δ 7.26; MeOH δ 3.30; DMSO δ 2.49) as standard. The NMR data of the synthesized examples, some of which are not disclosed in the following detailed charaterizations, are in agreements with their corresponding structural assignments.

Optical Rotation

Optical rotations of the purified enantiomers were measured with a Perkin-Elmer 241 polarimeter under the Na D line at room temperature. [α]D was calculated and presented with the solvent and concentration used (g/100 mL).

Elemental analyses were conducted by Robertson Microlit Labs, Madison N.J. The results of elemental analyses, if conducted but not disclosed in the following detailed charaterizations, are in agreements with their corresponding structural assignments.

The general synthesis of a compound of this invention is described below in Flow Diagrams I-X. This illustration of the synthesis of indane derived compounds could be applied to the synthesis of tetrahydronaphthalene derived compounds as well by substituting appropriate starting materials. The starting materials and/or intermediates are either commercially available or are prepared in similar manners as described in the literature procedures or the procedures described in the specific examples.

The right-hand portion of the compounds of Formula (I), the optionally substituted phenyl propenoate moiety, may be constructed by forming connection A or connection B, described further below. The left-hand portion may be constructed by forming connection C or connection D. These connections are followed by hydroxamic acid formation.

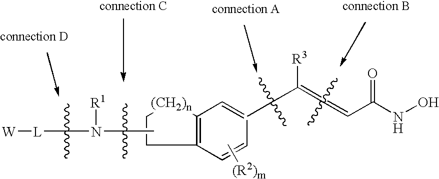

It should be apparent to those skilled in the art that the sequence of the synthetic steps is dependent on starting material availability and functional group compatibility and could vary from compound to compound (see, e.g., Table I, “Synthetic sequence” column for examples of the sequence of steps followed to provide the specific Compound Example). Protection and deprotection reactions could be involved in addition to the following reactions, as would be obvious to one skilled in the art. The groups and terms R¹, R², R³, R¹², m, L, and W used below are as defined previously unless specified otherwise.

Connection A

Connection A is the coupling of the optionally substituted indane portion of the molecule to the optionally substituted propenoate portion of the molecule. It can be formed by using metal-mediated cross-coupling reactions such as Heck Reaction as illustrated in Flow Diagram I.

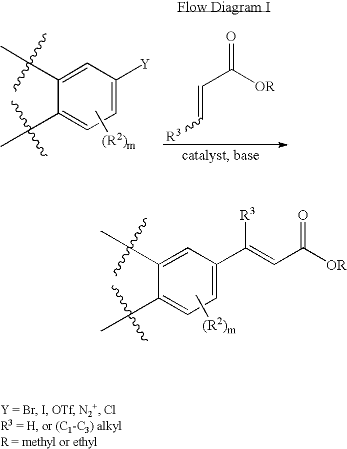

Alternatively, Connection A can be formed via the intermediate propynoate followed by halogenation of the propynoate as illustrated in Flow Diagram II. 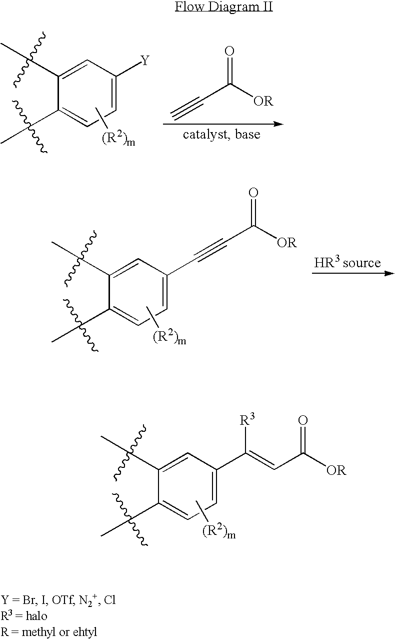
Connection B

Connection B is the coupling of the optionally substituted indane aldehyde or ketone to the acetate portion of the molecule. It can be formed by using olefination reaction such as Wittig reaction or Homer-Emmons reaction as illustrated in Flow Diagram III. 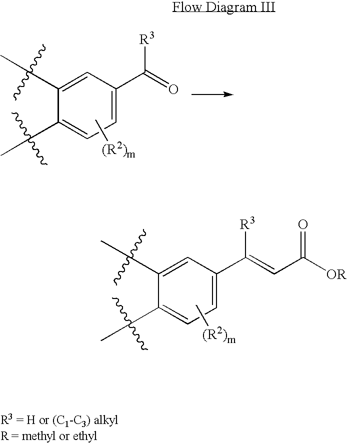
Connection C

Connection C is the coupling of the optionally substituted indanone to the optionally substituted amine. It can be formed via the reductive amination of optionally substituted indanones or a sequential reduction and displacement as illustrated in Flow Diagram IV. The optionally substituted amines are either commercially available or are prepared in similar manners as described in the specific procedures listed below or the literature procedures (for example, Journal of Organic Chemistry (2003), 68(12), 4938-4940.) 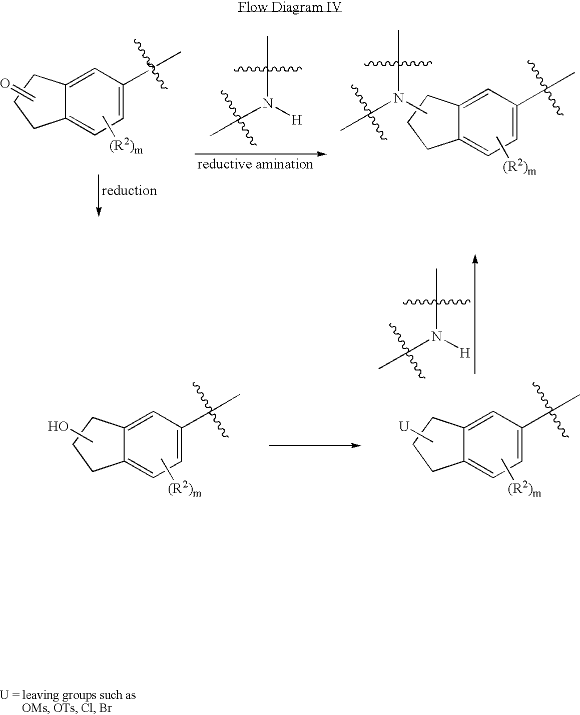
Connection D

Connection D is the coupling of the optionally substituted aminoindane to the optionally substituted alkyl groups. It can be formed via either the reductive amination or N-alkylation as illustrated in Flow Diagram V. 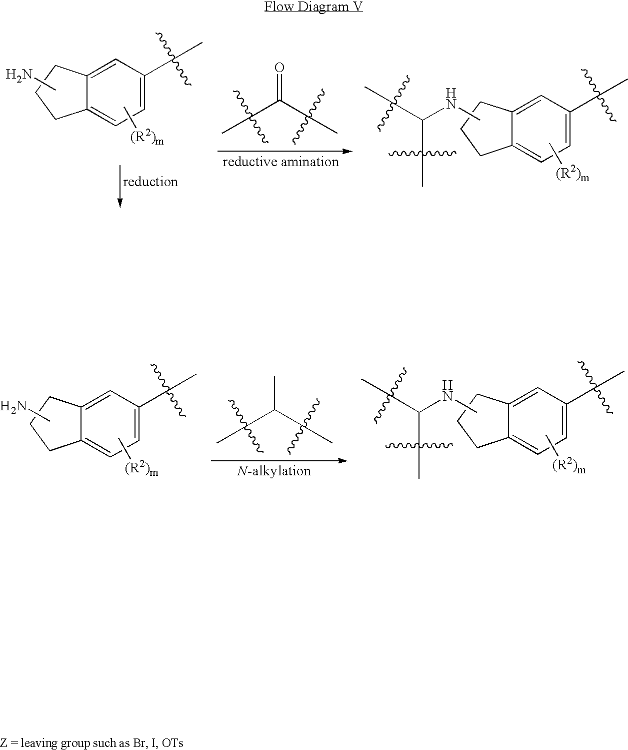
Hydroxamic Acid Formation

Hydroxamic acids could be formed via several pathways as illustrated in Flow Diagram VI. 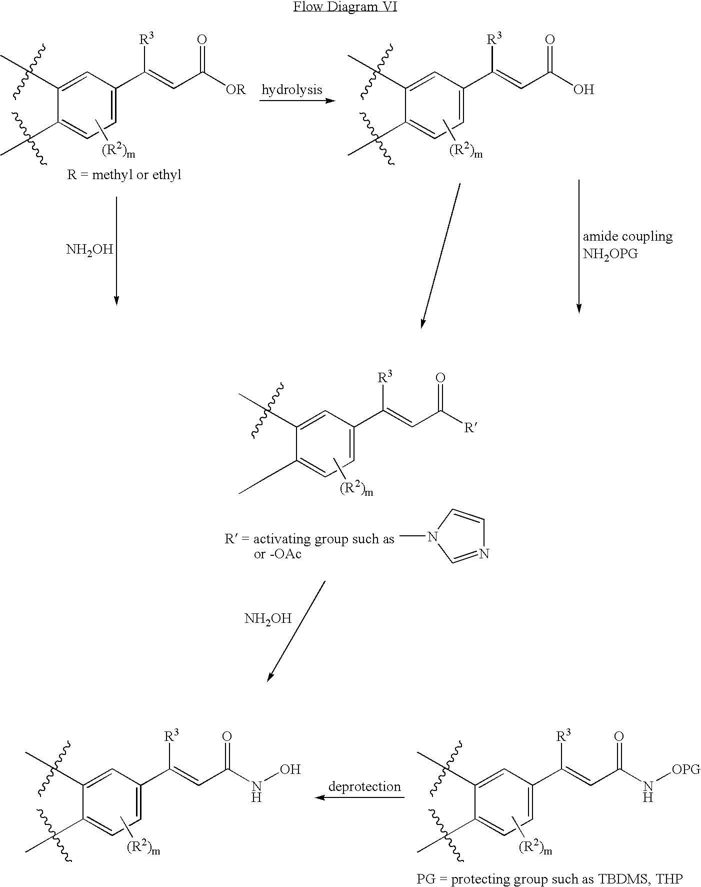
Further Manipulations

If the following functional groups are present in the molecule, the transformations listed in Flow Diagram VII could be conducted.

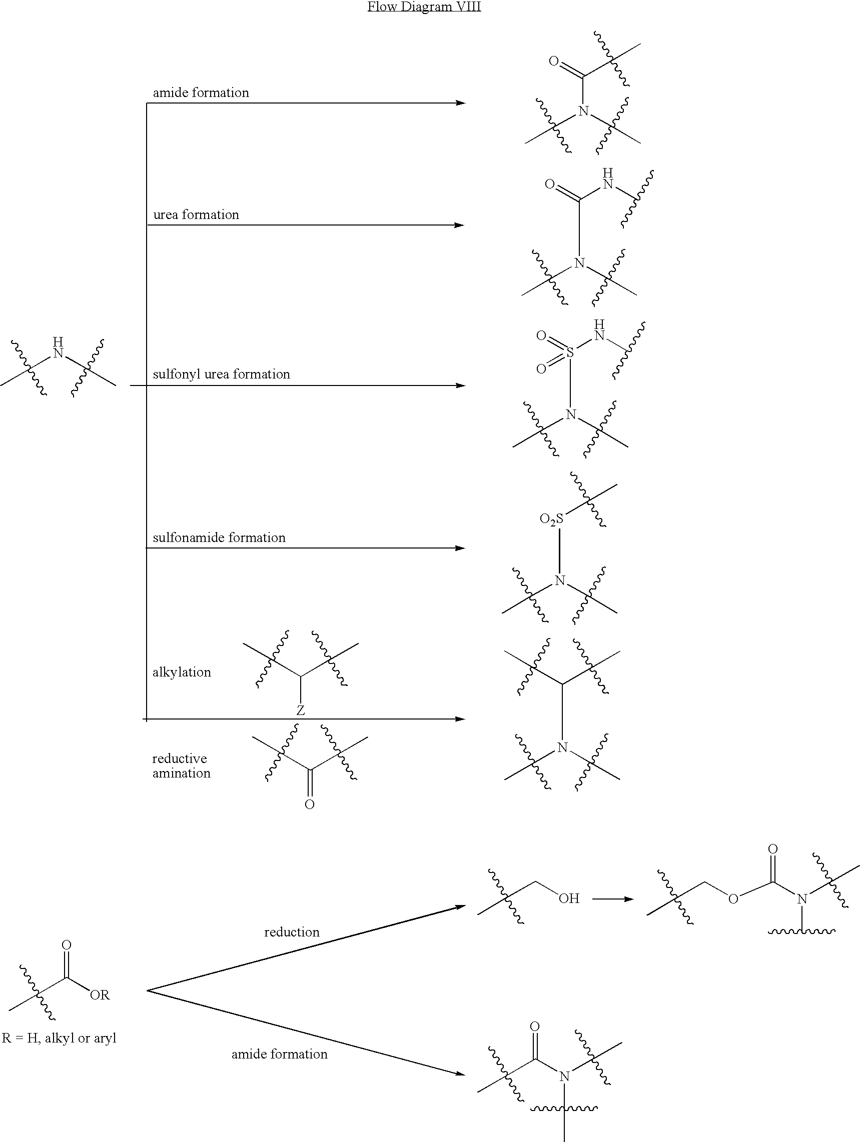

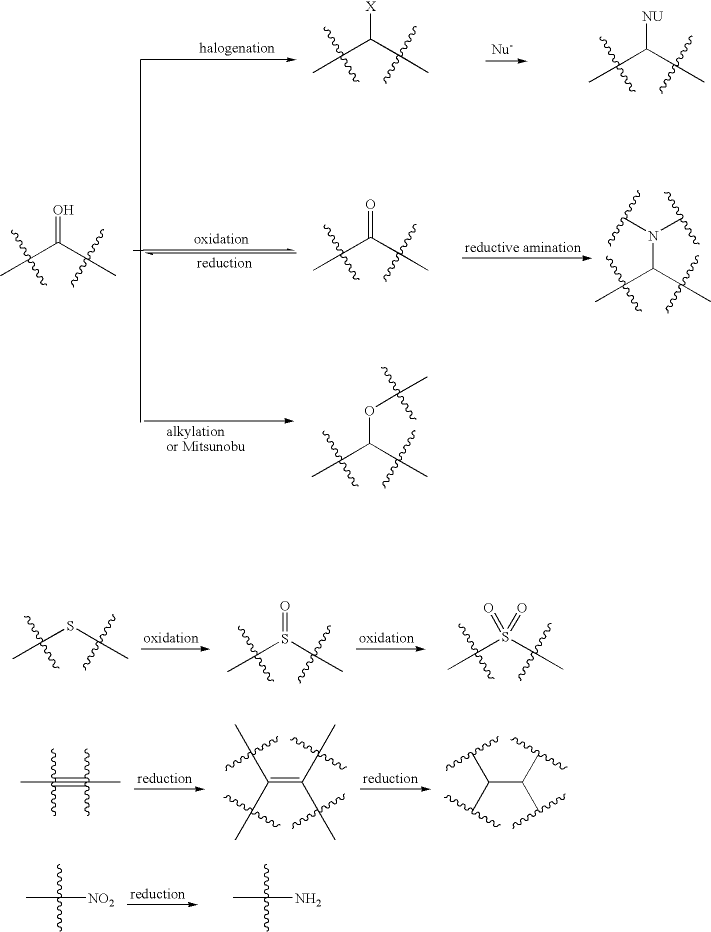

When R¹ is (C₁-C₆)alkyl optionally substituted with optionally substituted phenyl, optionally substituted pyrrolyl, optionally substituted pyrazolyl, or optionally substituted another heteroaryl, R¹ is often attached to its linked N atom via a reductive amination reaction between an aldehyde and optionally substituted amino-indane (Flow Diagram VIII). The aldehydes are either commercially available or are prepared in similar manners as described in the literature procedures [for example, Canadian Journal of Chemistry (1990), 68(5), 791-4; Canadian Journal of Chemistry (1995), 73(5), 675-84; Tetrahedron (2001), 57(15), 3063-3067. Canadian Journal of Chemistry (1978), 56(5), 654-7; Canadian Journal of Chemistry (1981), 59(17), 26736; Tetrahedron Letters (2002), 43(20), 3673-3675; Canadian Journal of Chemistry (1980), 58(23), 2527-30; Bioorganic & Medicinal Chemistry Letters (1994), 4(21), 2627-30; Chemicke Zvesti (1983), 37(2), 251-62.], the general synthetic sequence shown in Flow Diagram IX and X, or the specific procedures below. For the purpose of clear illustration, only the 1,4-substitution pattern is shown in Flow Diagrams IX and X. However, the synthetic sequence can be applied to 1, 2- or 1, 3-substitution pattern as well.

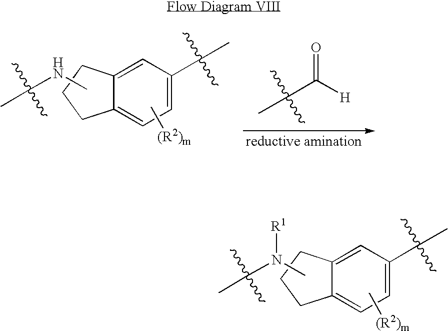

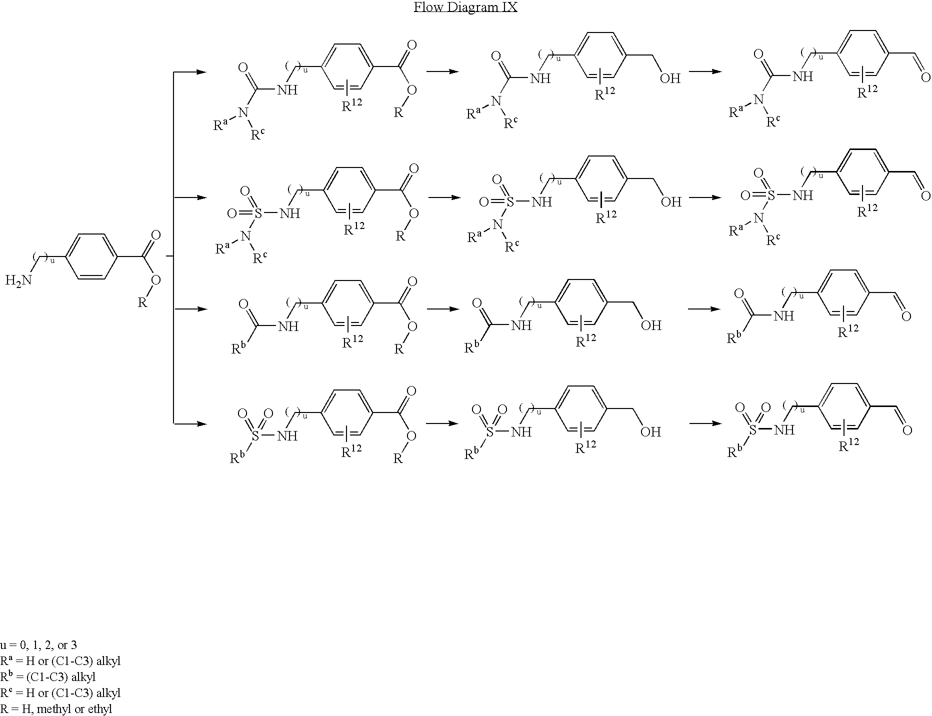

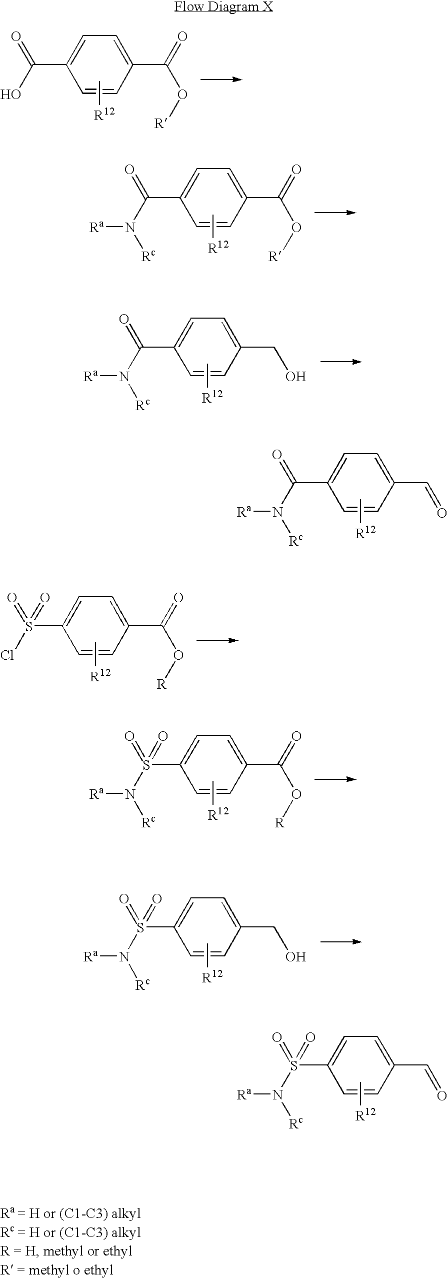

The following specific examples are presented to illustrate the invention, but they should not be construed as limiting the scope of the invention in any way. In the tables listing the intermediates, those compounds that have characterization data such as HPLC retention time, M+H mass spectroscopy data, TLC R_{f} value, or NMR data listed were actually synthesized. Those that do not have characterization data were not synthesized; however, they can be synthesized by following procedures that are well known to those skilled in the art and/or procedures that are disclosed in this application.

### EXPERIMENTAL EXAMPLES OF THE INVENTION

### Intermediate A

### tert-Butyl 3-(2-bromoethyl)-1H-indole-1-carboxylate

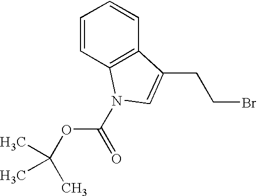

Ethyl 1H-indol-3-ylacetate (2.5 9, 12.3 mmol) was dissolved in THF (60 mL) and to the resulting solution was added Di-tert-butyl carbonate (2.9 g, 16.6 mmol), Et₃N (1.89 mL), and DMAP (150 mg, 1.23 mmol). The reaction was stirred for 16 h at rt. The solvent was removed under vacuum and the residue was re-dissolved in Et₂O and saturated NaHCO₃ was added and the mixture was stirred vigorously for 30 min. The organic phase was collected, and dried over Na₂SO₄. The solvent was removed by vacuum. The crude product was purified further by passing it through a plug of silica using Et₂O as eluent. The solvent was removed under vacuum to give tert-butyl 3-(2-ethoxy-2-oxoethyl)-1H-indole-1-carboxylate as an oil (3.73 g, 99%): ¹H NMR (CDCl₃) δ 8.14 (m, 1H), 7.54 (m, 2H), 7.32 (m, 1H), 7.24 (m, 2H), 4.19 (q, 2H), 3.71 (s, 1H), 1.68 (s, 9H), 1.29 (t, 3H).

The following intermediate compounds are synthesized in a similar manner:

| Intermediate | Structure | ¹H NMR |
|---|---|---|
| A1 | 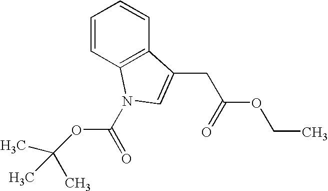 | (CDCl₃) δ 8.14 (m, 1H), 7.55 (m, 2H), 7.22-7.34 (m, 2H), 4.19 (q, 2H), 3.72 (s, 2H), 1.68 (s, 9H), 1.29 (t, 3H) |
| A2 | 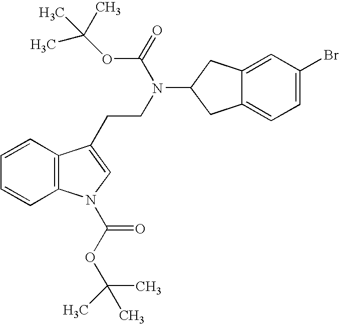 | (CD₂Cl₂) δ 8.10 (m, 1H), 7.34 (m, 2H), 7.29 (m, 3H), 7.21 (m, 1H), 7.08 (m, 1H), 3.08 (m, 6H), 2.92 (m, 3H), 1.67 (s, 9H), 1.46 (br s, 9H). |
| A3 | 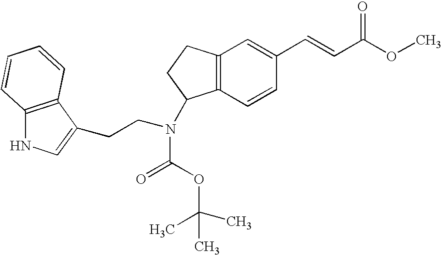 | HPLC RT: 3.87 (A) M + H: 460.9 |

### Intermediate B

### tert-Butyl 3-(2-oxoethyl)-1H-indole-1-carboxylate

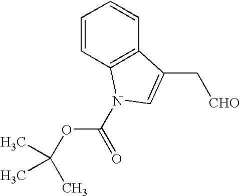

Intermediate A1 (tert-Butyl 3-(2-ethoxy-2-oxoethyl)-1H-indole-1-carboxylate) (1.5 g. 4.94 mmol) was dissolved in THF(30 mL) and the resulting mixture was cooled to −78° C. DIBAL (1M in Hex, 738 mg, 5.19 mmol) was added dropwise to the solution. No reaction occurred after the addition of the first equivalent of DIBAL was added. More DIBAL was added (1107 mg, 7.79 mmol) to the reaction. The reaction was then quenched with MeOH at −78° C. to limit alcohol formation even though there was still starting material. A saturated solution of Rochelle's salt (sodium potassium tartrate) was added to the reaction. This mixture was extracted with EtOAc. The organic layer was collected and dried over Na₂SO₄. The solvent was then removed under vacuum. The crude product was purified by silica gel chromatography using 5-10% EtOAc in Hex as eluent to give tert-butyl 3-(2-oxoethyl)-1H-indole-1-carboxylate as an oil (215 mg, 17% yield): ¹H-NMR δ (CD₂Cl₂) 9.78 (m, 1H), 8.18 (m, 1H), 7.61 (m, 1H), 7.47 (m, 1H), 7.37 (m, 1H), 7.27 (m, 1H), 3.80 (m, 2H), 1.70 (m, 9H).

### Intermediate C

### 5-Bromo-2,3-dihydro-1H-inden-2-ylamine hydrochloride

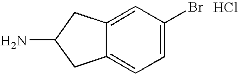

2-Aminoindane hydrochloride (4.12 g, 24.3 mmol) and water (40 mL) were mixed and the resulting mixture was heated to 60° C. Bromine (4.07 g, 25.5 mmol) was added dropwise over 45 min and the reaction mixture was stirred for an additional hour before it was cooled in an ice-bath. The solid formed was filtered and washed with water, Et₂O, and then dried under vacuum to give 5-bromo-2-indane as the hydrochloride salt (3.8 g, 63%). ¹H-NMR: (DMSO-d6) δ 8.08 (br s, 3H), 7.49 (m, 1H), 7.36 (m, 1H), 7.23 (d, 1 H), 4.00 (br s, 1H), 3.25 (m, 2H), 2.92 (m, 2H).

### Intermediate D

### Methyl (2E)-3-(1-oxo-2,3-dihydro-1H-inden-5-yl)-2-prodenoate

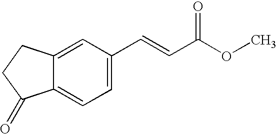

To a solution of 5-bromo-1-indanone (1.50 g, 71.0 mmol) in CH₃CN (45 mL) and Et₃N (45 mL) was added Pd(OAc)₂ (0.957 g, 4.2 mmol), PPh₃ (2.793 g, 10.7 mmol), methyl acrylate (16 mL, 177.7 mmol). The reaction mixture was heated to 85 ° C. under argon for 16 h. The mixture was cool to rt and the solvent was evaporated in vacuo. The resulting black residue was taken up in CH₂Cl₂ and filtered through Celite. The filtrate was washed with 2N HCl, saturated aqueous NaHCO₃, and brine. It was then dried over MgSO₄ and concentrated in vacuo. The resulting yellow crude solid was triturated with Et₂O to yield methyl (2E)-3-(1-oxo-2,3-dihydro-1H-inden-5-yl)-2-propenoate (13.23 g, 86%): TLC R_{f}=0.35 (25% EtOAc in Hex), ¹H-NMR (CD₂Cl₂) δ 7.72 (d, 1H), 7.7(s, 1H), 7.65 (s, 1H), 7.54-7.57(m, 1H), 6.5 (d, 1H), 3.80 (s, 3H), 3.16 (t, 2H) and 2.70 (t, 2H).

The following compounds are synthesized in a similar manner:

| | | HPLC RT | |
|---|---|---|---|
| | | (min) | |
| Intermediate | Structure | (method) | M + H |
| D1 | 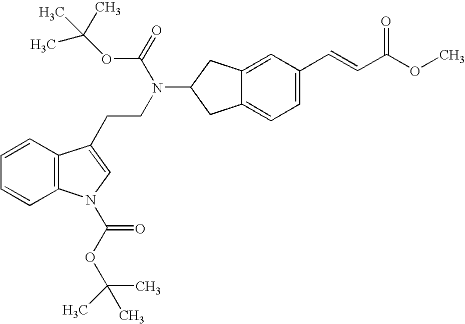 | 4.98 (A) | 583.1 (+Na) |
| D2 | 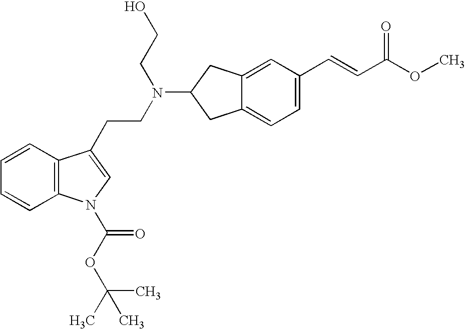 | 3.06 (B) | 505.6 |
| D3 | 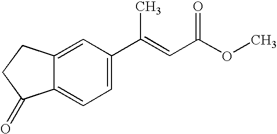 | 2.30 (B) | 231.3 |
| D4 | 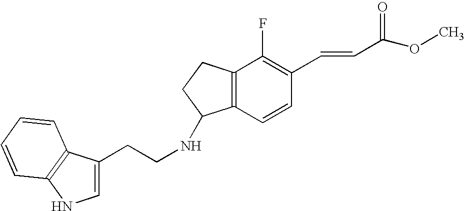 | 2.31 (A) | 379.0 |
| D5 | 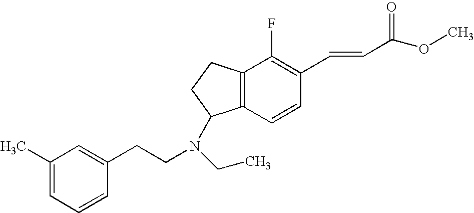 | ¹H-NMR (DMSO-d6) δ7.79 (d, 1H), 7.35 (t, 1H), 7.13 (t, 1H), 6.95 (m, 4H), 6.51 (d, 1H), 4.58 (t, 1H), 3.79 (s, 3H), 2.99 (m, 1H), 2.77 (m, 2H), 2.62 (m, 5H), 2.31 (s, 3H), 2.21 (m, 1H), 2.01 (m, 1H), 1.09 (t, 3H). | |

### Intermediate E

### Methyl (2E)-3-(1-{[2-(1H-indol-3-yl)ethyl]amino}-2,3-dihydro-1H-inden-5-yl-2-propenoate

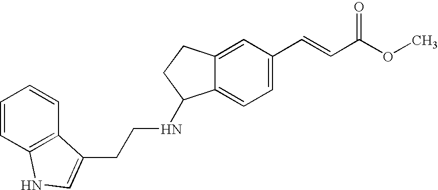

A mixture of Intermediate D [methyl ((2E)-3-(1-oxo-2,3-dihydro-1H-inden-5-yl)-2-propenoate)] (1.00 g, 4.62 mmol), tryptamine (0.78 g, 4.86 mmol), toluenesulfonic acid (0.02 g, 0.14 mmol) and toluene (25 mL) in a 100 mL round bottle flask with a Dean-Stark condenser was heated to reflux for 3 h. The crude mixture was concentrated under vacuum to give a black residue. It was dissolved with dichloroethane (20 mL) and NaBH(OAc)₃ (0.98 g, 4.62 mmol) was added. The mixture was stirred overnight at rt. The reaction was quenched with saturated NaHCO₃ and extracted with CH₂Cl₂ twice. The combined organic layer was washed with brine and dried over Na₂SO₄. The solvent was evaporated to give the crude product as a dark residue. It was purified with 40 M Biotage eluting with MeOH (with 2M NH₃)/CH₂Cl₂ (5/95) to obtain methyl (2E)-3-(1-{[2-(1H-indol-3-yl)ethyl]amino}-2,3-dihydro-1H-inden-5-yl)-2-propenoate as a brown solid (0.68 g, 40%): LC/MS [M+H] 361.0, RT 2.27 min (method A). ¹H-NMR (DMSO-d6) δ 10.76 (s, 1H), 7.63 (d, 1H), 7.56 (s, 1H), 7.49 (m, 2H), 7.34 (m, 2H), 7.13 (d,1H), 7.05 (m, 1H), 6.95 (m, 1H), 6.58 (d, 1H), 4.23 (t, 1H), 3.70 (s, 3H), 2.92 (m, 5H), 2.73 (m, 1H), 2.33 (m, 1H), 1.80 (m, 1H).

The following compounds are synthesized in a similar manner. For intermediates E4, E5, E6, and E7, a mixture of n-butanol and toluene is used as the solvent for the Schff base formation.

Intermediate E is also formed in a similar manner as described in the synthesis of intermediate Q with the alternative work up as an HCl salt.

| | | HPLC RT | |
|---|---|---|---|
| | | (min) | |
| Intermediate | Structure | (method) | M + H |
| E1^{a, b} | 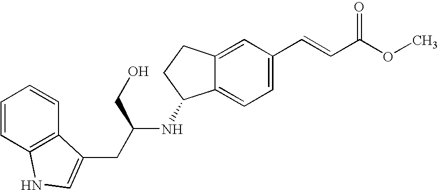 | 2.11 (A) | 391.0 |
| E2^{a, b} | 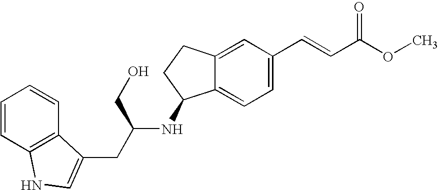 | 2.13 (A) | 391.0 |
| E3^{a} | 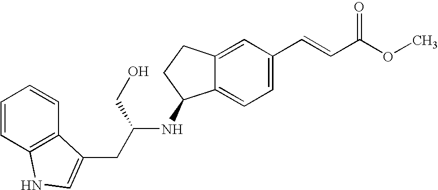 | 2.12 (A) | 391.0 |
| E4 | 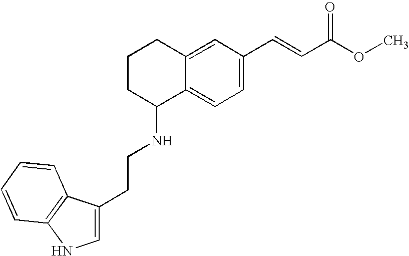 | 2.24 (A) | 375.0 |
| E5^{a} | 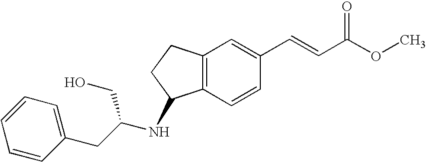 | 2.26 (A) | 351.9 |
| E6^{a} | 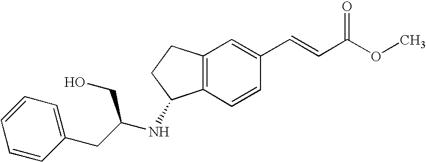 | 2.15 (A) | 351.9 |
| E7^{a} | 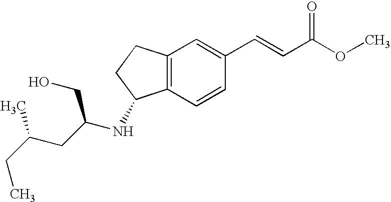 | 2.02 (A) | 318.0 |
| E8 | 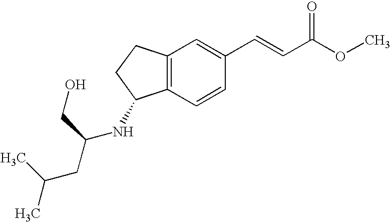 | 1.99 (A) | 317.9 |

a. The absolute configuration of the indane chiral center is tentatively assigned based the facial selectivity observed by Stalker et al. in Tetrahedron, 2002, 58, 4837-4849.
b. E1 and E2 are formed in the same reaction and E2 is isolated as the minor isomer.

### Intermediate F

### 5-Bromo-N-2-{[tert-butyl(dimethyl)silyl[oxy}ethyl)-2-indanamine

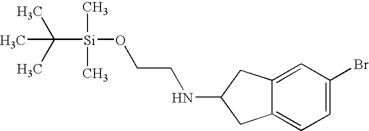

Intermediate C (5- Bromo-2-indanamine) (226 mg, 1.07 mmol), {[tert-butyl(dimethyl)silyl]oxy} acetaldehyde (186 mg, 1.07 mmol) and dichloroethane (10 mL) was placed in a flask along with AcOH (73 uL, 1.28 mmol), followed by the immediate addition of NaBH(OAc)₃ (316 mg, 1.49 mmol). The mixture was stirred for 1 h at rt. The reaction was quenched with saturated NaHCO₃, and extracted with CH₂Cl₂. The organic layer was collected and dried over Na₂SO₄. The solvent was removed under vacuum to give 5-bromo-N-(2-{[tert-butyl(dimethyl)silyl]oxy}ethyl)-2-indanamine as an oil (384 mg, 97%). It was used for further reactions without purification: ¹H-NMR (CD₂Cl₂) δ 7.26 (m, 1H), 7.17 (m, 1H), 6.99 (m, 1H), 3.64 (m, 2H), 3.56 (m, 1H), 3.04 (m, 2H), 2.57-2.70 (m, 4H), 0.83 (s, 9H), 0.01 (s, 6H).

The following compounds are synthesized in a similar manner.

| | | HPLC RT | |
|---|---|---|---|
| | | (min) | |
| Intermediate | Structure | (method) | M + H |
| F1 | 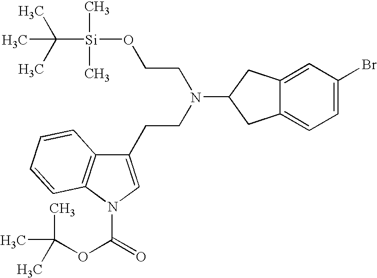 | 3.56 (A) | 613.1 |
| F2 | 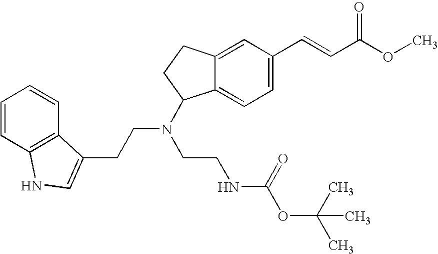 | 2.60 (A) | 504.1 |
| F3 | 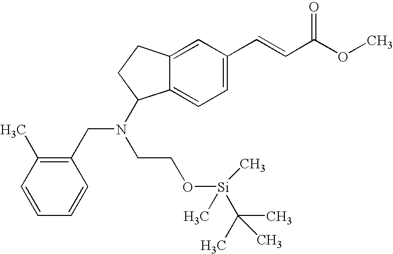 | 3.64 (A) | 480.1 |
| F4 | 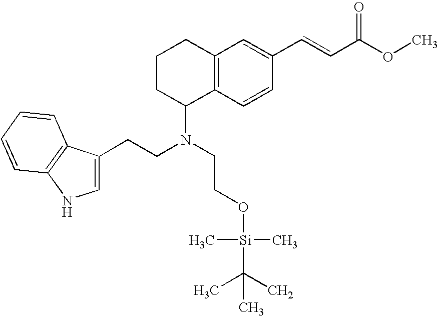 | 2.94 (A) | 533.0 |
| F5 | 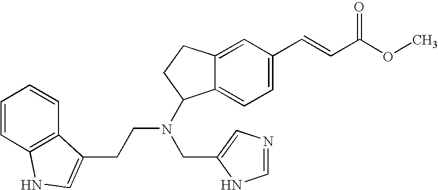 | 2.08 (A) | 441.1 |
| F6 | 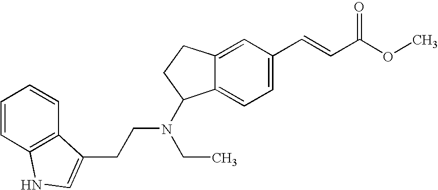 | 2.30 (A) | 389.1 |
| F7 | 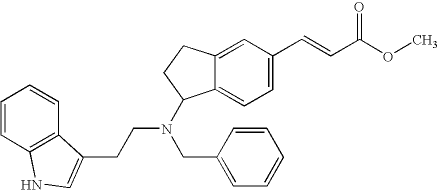 | 2.53 (A) | 451.0 |
| F8 | 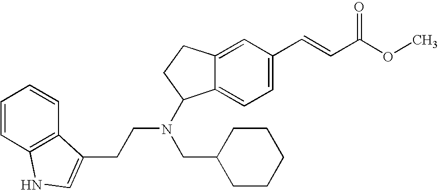 | 2.69 (A) | 457.2 |
| F9 | 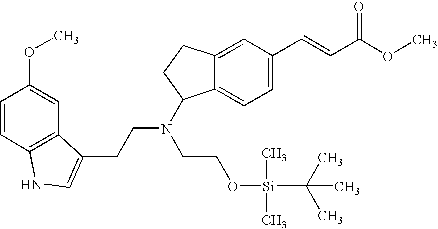 | 2.96 (A) | 549.2 |
| F10 | 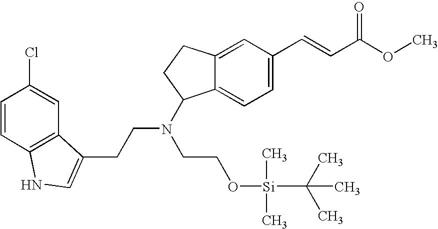 | 3.52 (A) | 353.1 |
| F11 | 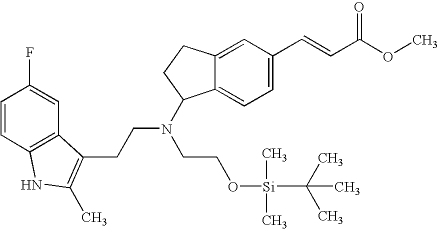 | 3.52 (A) | 351.1 |
| F12 | 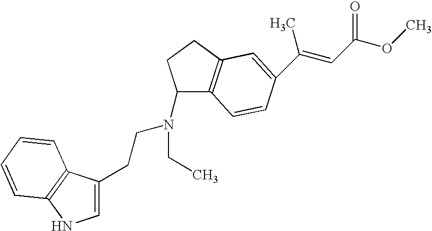 | 2.49 (A) | 403.0 |
| F13 | 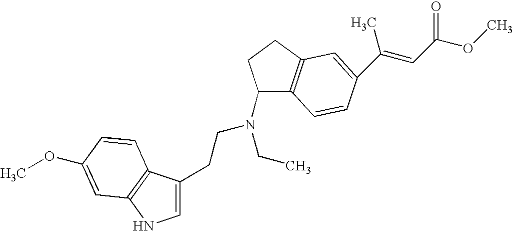 | 2.86 (A) | 433.2 |
| F14 | 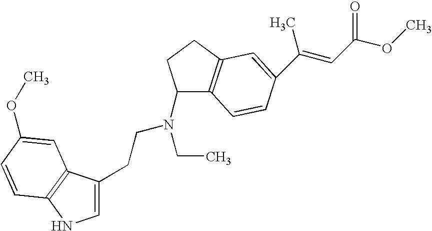 | 2.82 (A) | 433.2 |
| F15 | 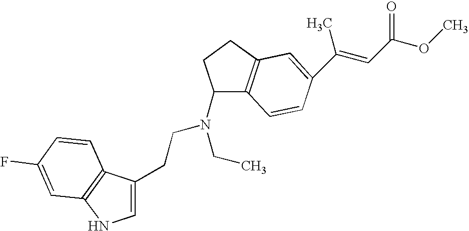 | 2.19 (B) | 421.2 |
| F16 | 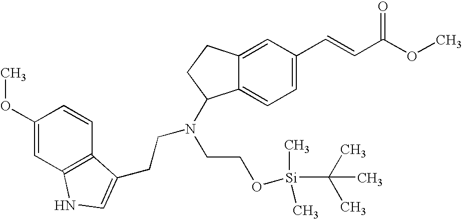 | 2.91 (A) | 549.4 |
| F17 | 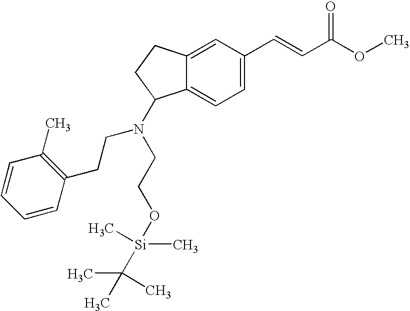 | 3.63 (A) | 494.1 |
| F18 | 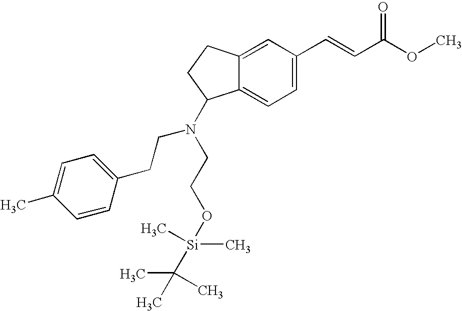 | 3.63 (A) | 494.1 |
| F19 | 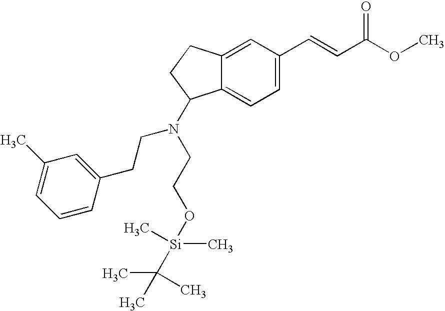 | 3.63 (A) | 494.1 |
| F20 | 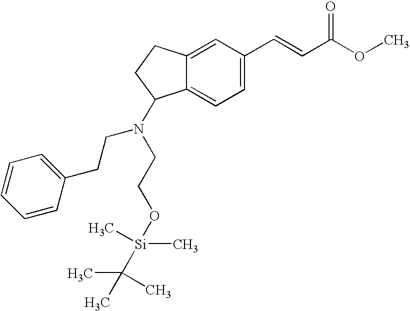 | 3.02 (A) | 480.1 |
| F21 | 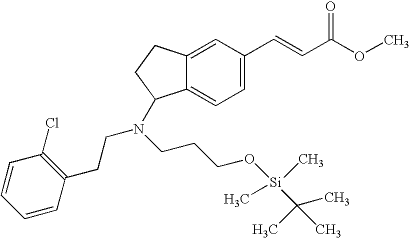 | 3.24 (A) | 528.1 |
| F22 | 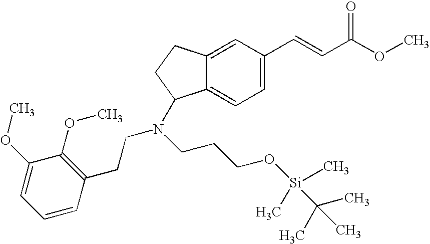 | 3.07 (A) | 554.2 |
| F23 | 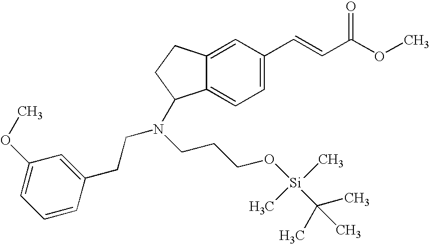 | 3.04 (A) | 524.2 |
| F24 | 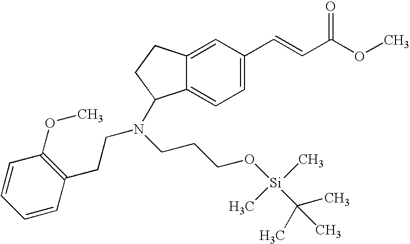 | 3.09 (A) | 524.2 |
| F25 | 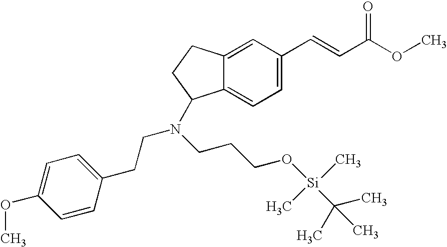 | 3.03 (A) | 524.2 |
| F26 | 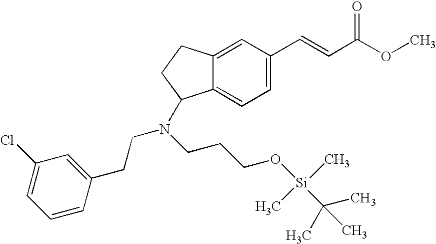 | 3.27 (A) | 528.1 |
| F27 | 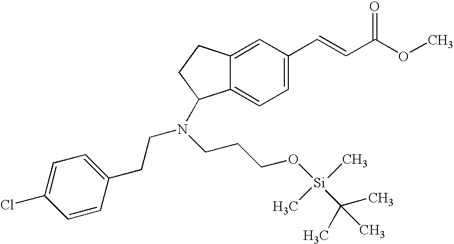 | 3.14 (A) | 528.3 |
| F28 | 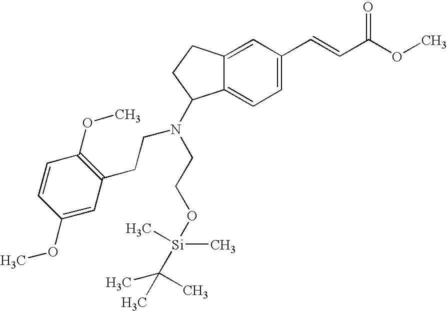 | 3.02 (A) | 540.1 |
| F29 | 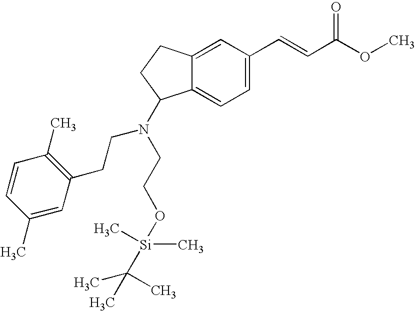 | 3.16 (A) | 508.2 |
| F30 | 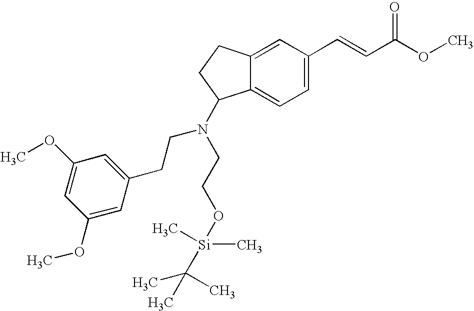 | 3.00 (A) | 540.1 |
| F31 | 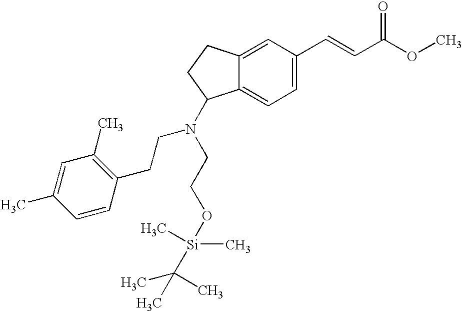 | 3.16 (A) | 508.2 |
| F32 | 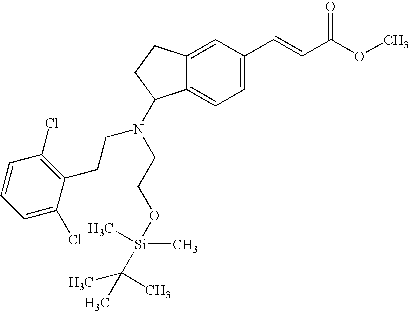 | 3.22 (A) | 548.2 |
| F33 | 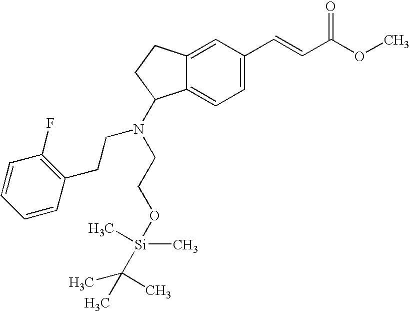 | 3.03 (A) | 498.1 |
| F34 | 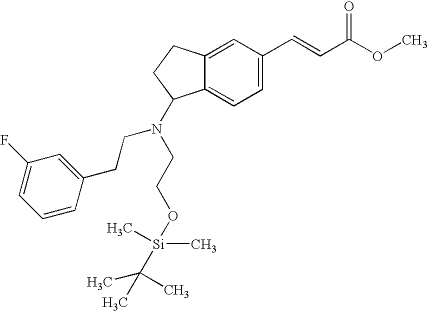 | 3.02 (A) | 498.1 |
| F35 | 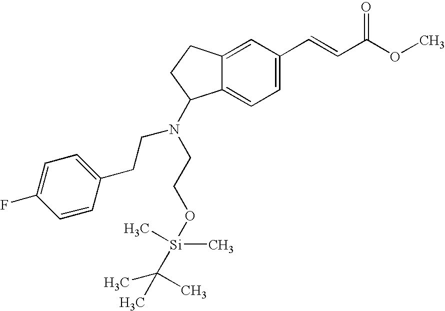 | 3.01 (A) | 498.1 |
| F36 | 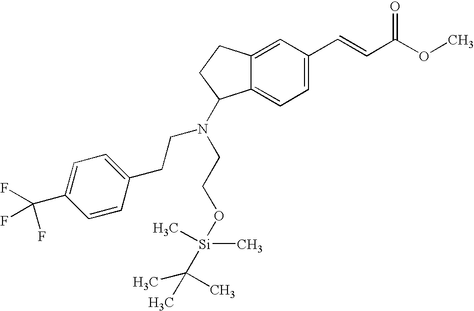 | 3.18 (A) | 548.1 |
| F37 | 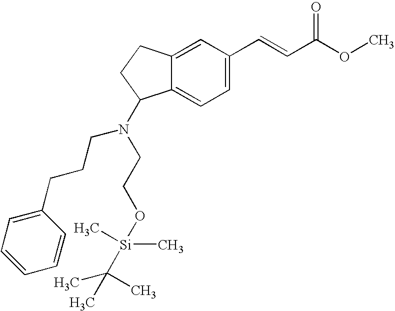 | 3.17 (A) | 494.2 |
| F38 | 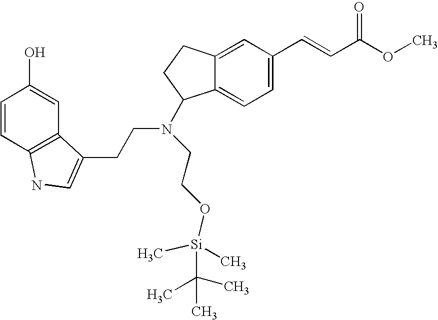 | 2.73 (A) | 534.7 |
| F39 | 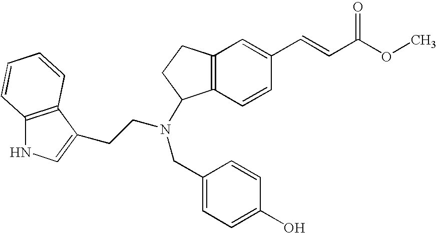 | 2.37 (A) | 467.1 |
| F40 | 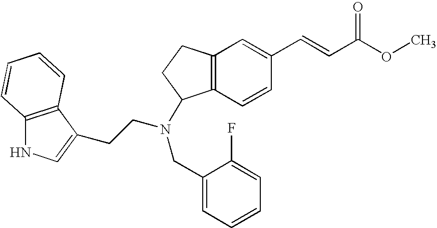 | 2.59 (A) | 469.0 |
| F41 | 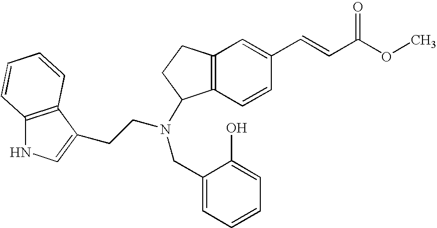 | 2.50 (A) | 467.0 |
| F42 | 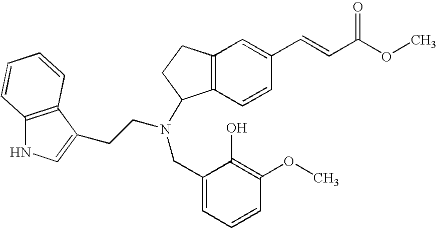 | 2.58 (A) | 497.1 |
| F43 | 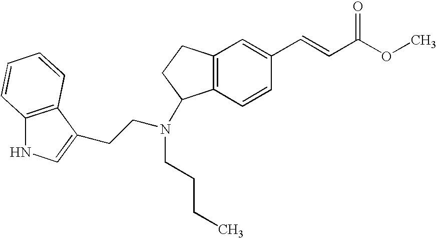 | 2.50 (A) | 417.2 |
| F44 | 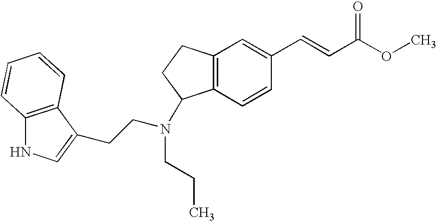 | 2.38 (A) | 403.1 |
| F45 | 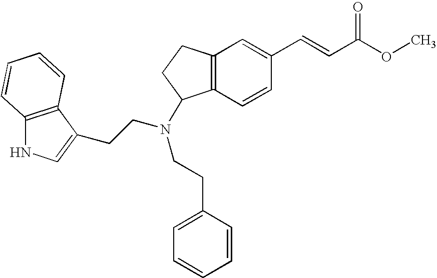 | 2.62 (A) | 465.1 |
| F46 | 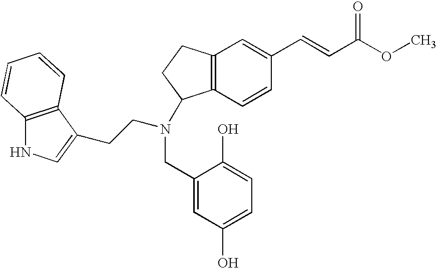 | 2.45 (A) | 483.0 |
| F47 | 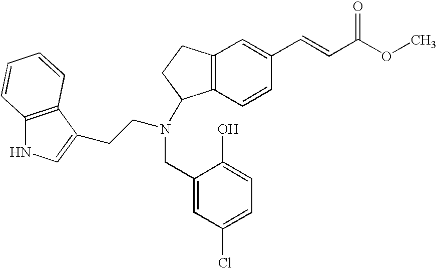 | 2.75 (A) | 501.1 |
| F48 | 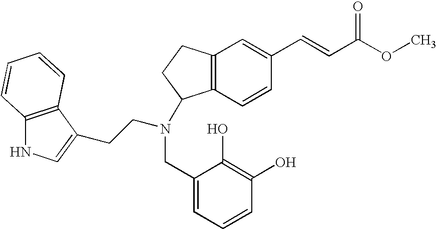 | 2.53 (A) | 483.1 |
| F49 | 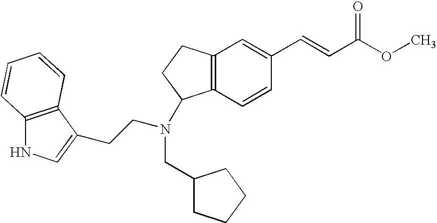 | 2.70 (A) | 443.1 |
| F50 | 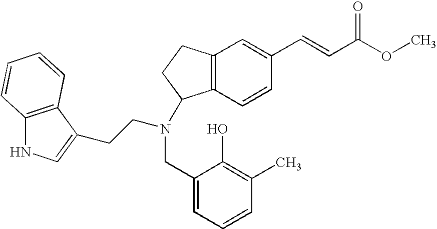 | 2.71 (A) | 481.1 |
| F51 | 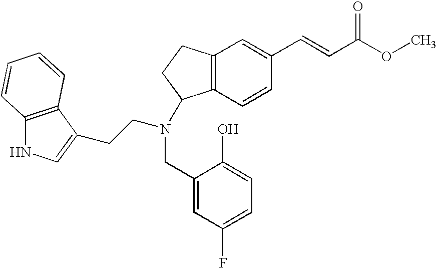 | 2.65 (A) | 485.1 |
| F52 | 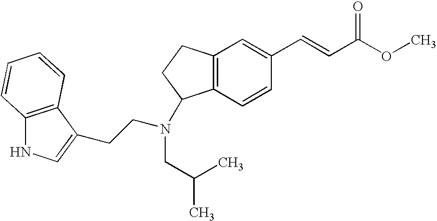 | 2.55 (A) | 417.1 |
| F53 | 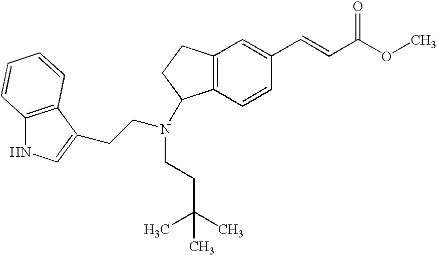 | 2.64 (A) | 445.2 |
| F54 | 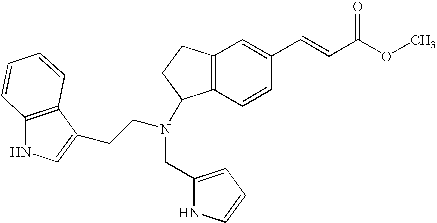 | 2.56 (A) | 439.9 |
| F55 | 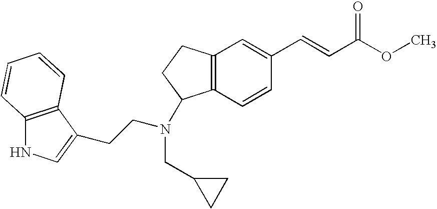 | 2.43 (A) | 415.1 |
| F56 | 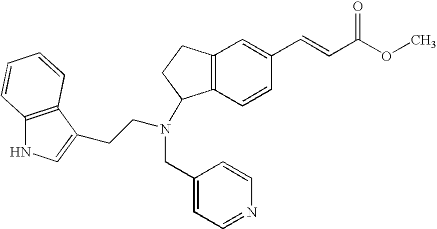 | 2.44 (A) | 452.2 |
| F57 | 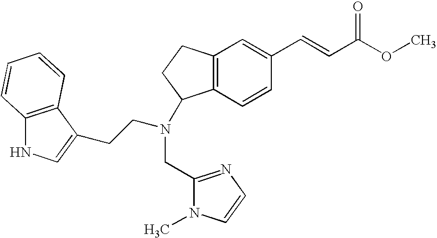 | 2.64 (A) | 455.1 |
| F58 | 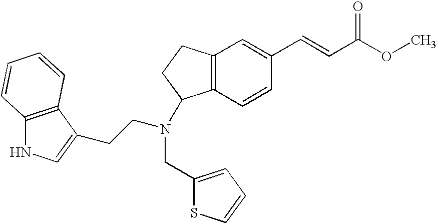 | 2.49 (A) | 457.1 |
| F59 | 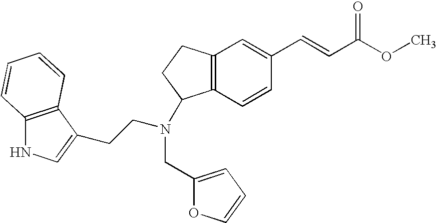 | 2.48 (A) | 441.0 |
| F60 | 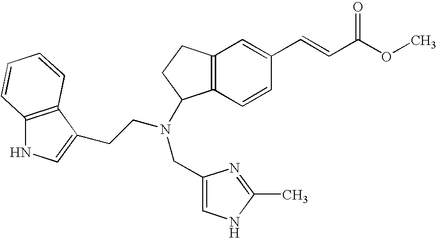 | 2.02 (A) | 455.2 |
| F61 | 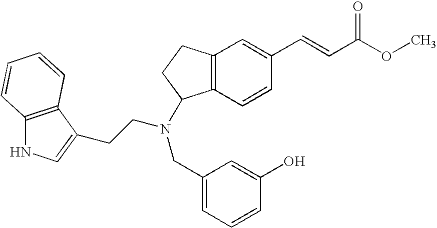 | 2.38 (A) | 467.1 |
| F62 | 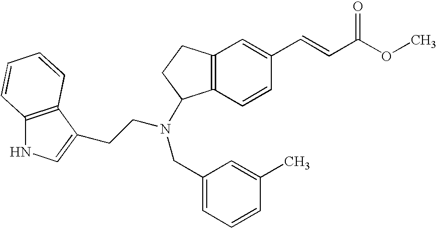 | 2.63 (A) | 465.1 |
| F63 | 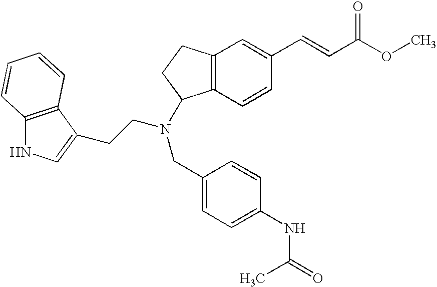 | 2.50 (A) | 508.1 |
| F64 | 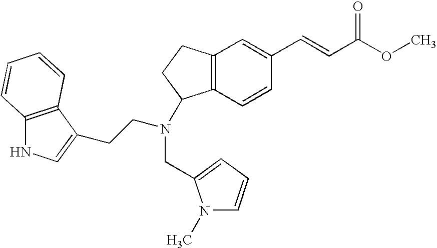 | 2.33 (A) | 454.1 |
| F65 | 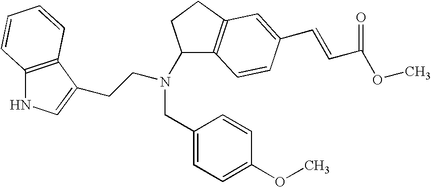 | 2.69 (A) | 481.0 |
| F66 | 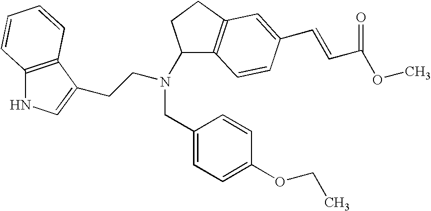 | 2.68 (A) | 495.1 |
| F67 | 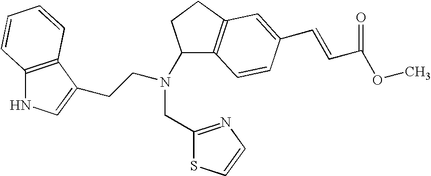 | 2.61 (A) | 458.0 |
| F68 | 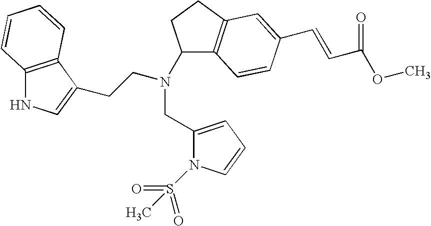 | 2.57 (A) | 518.0 |
| F69 | 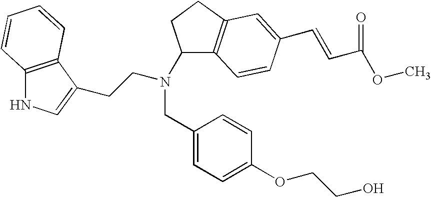 | 2.53 (A) | 511.1 |
| F70 | 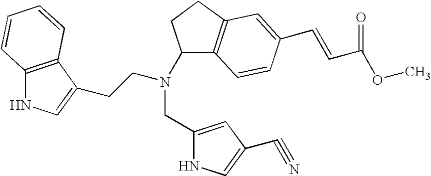 | 2.21 (A) | 464.9 |
| F71 | 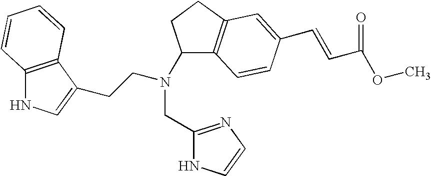 | 2.66 (A) | 441.1 |
| F72 | 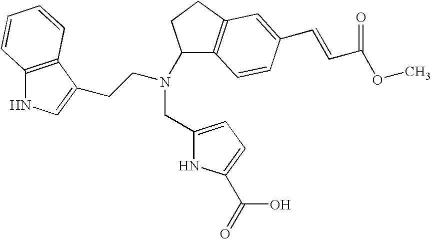 | 2.34 (A) | 484.0 |
| F73 | 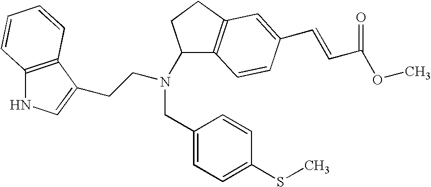 | 2.82 (A) | 497.0 |
| F74 | 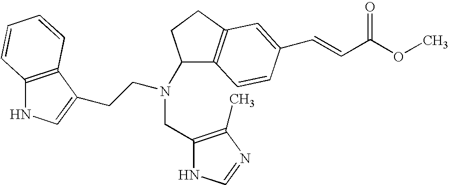 | 2.23 (A) | 455.1 |
| F75 | 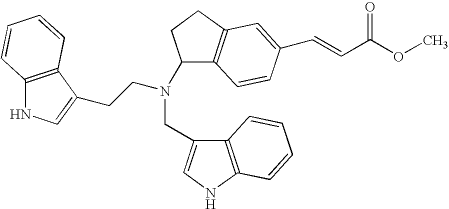 | 2.73 (A) | 490.0 |
| F76 | 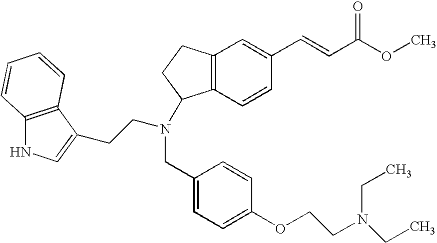 | 2.19 (A) | 566.2 |
| F77 | 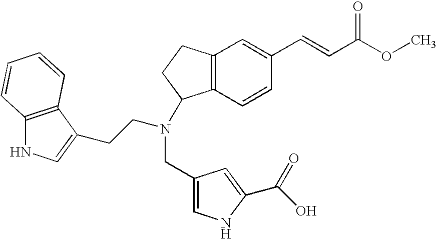 | 2.36 (A) | 484.0 |
| F78 | 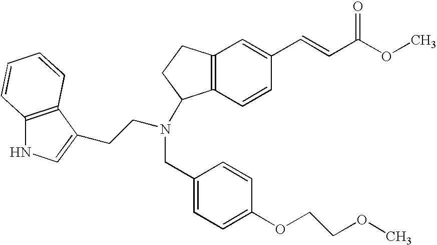 | 2.66 (A) | 525.1 |
| F79 | 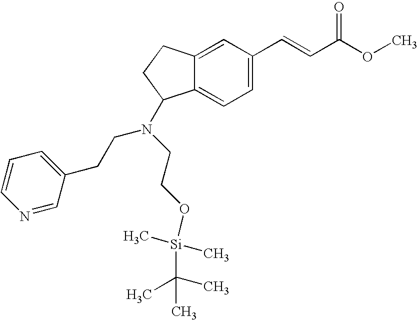 | 2.51 (A) | 481.1 |
| F80 | 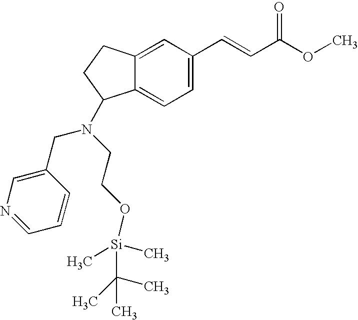 | 2.92 (A) | 467.2 |
| F81 | 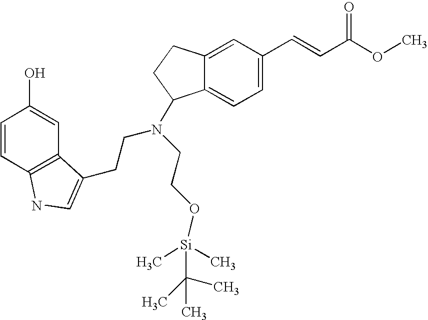 | 2.73 (A) | 534.7 |
| F82 | 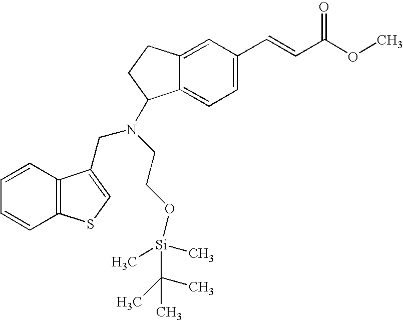 | 3.35 (A) | 522.1 |
| F83 | 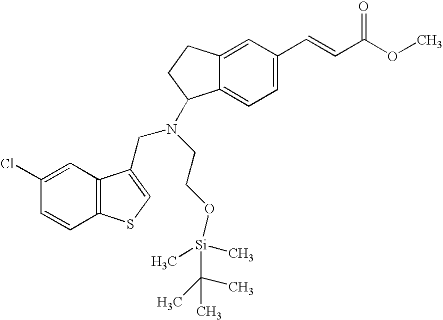 | 3.57 (A) | 556.1 |
| F84 | 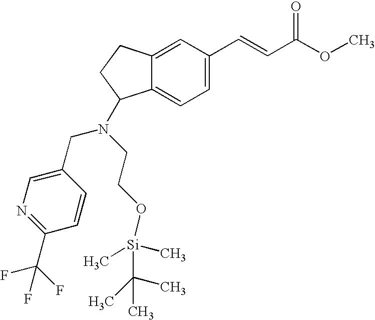 | 3.70 (A) | 535.0 |
| F85 | 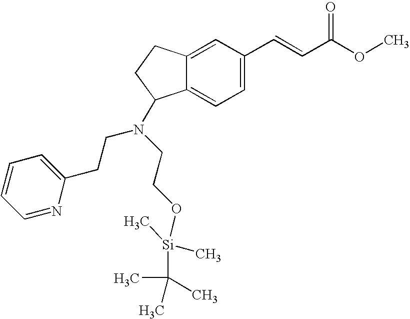 | 2.96 (A) | 481.2 |
| F86 | 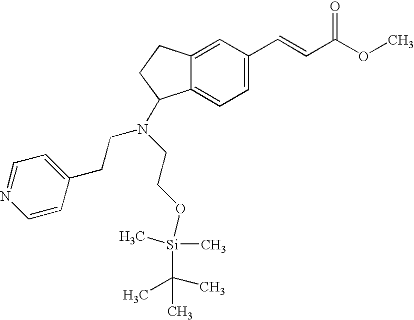 | 2.42 (A) | 481.5 |
| F87 | 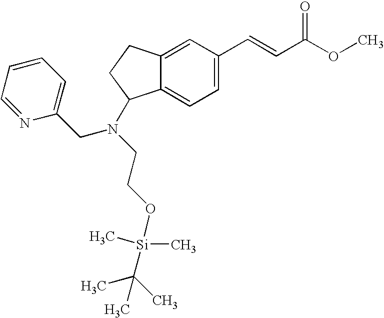 | 2.96 (A) | 467.3 |
| F88 | 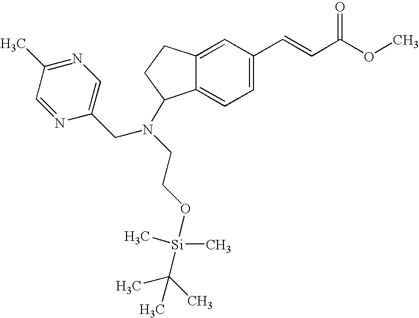 | 3.22 (A) | 482.1 |
| F89 | 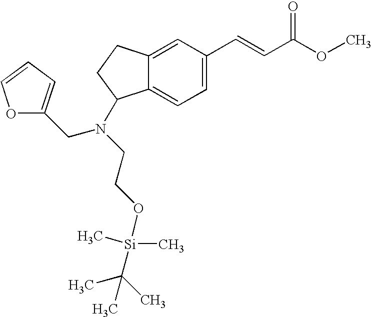 | 2.88 (A) | 456.1 |
| F90 | 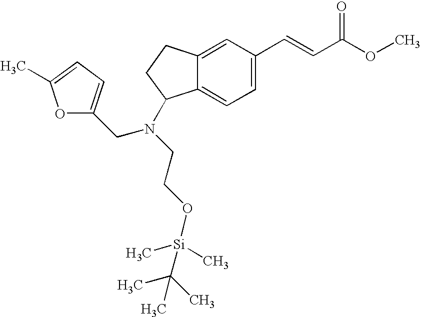 | 2.96 (A) | 470.1 |
| F91 | 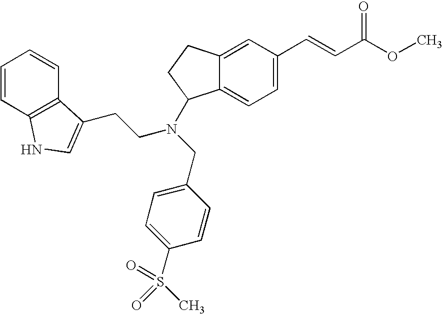 | 2.56 (A) | 529.0 |
| F92 | 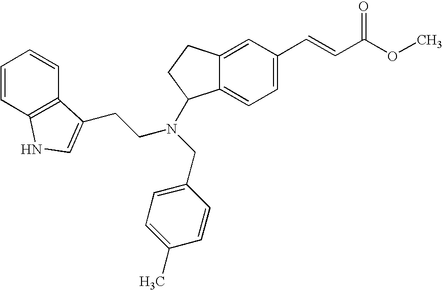 | 2.71 (A) | 465.0 |
| F93 | 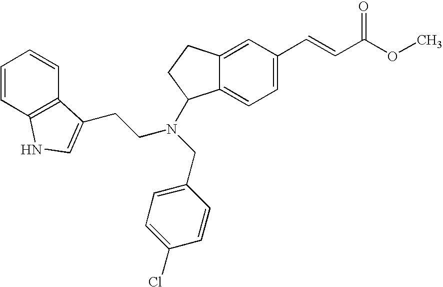 | 2.78 (A) | 485.1 |
| F94 | 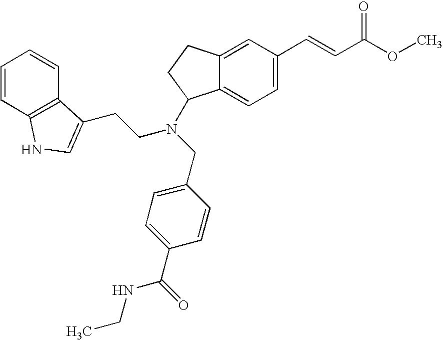 | 2.12 (A) | 522.0 |
| F95 | 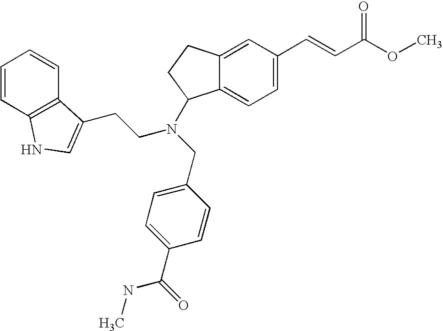 | 2.02 (A) | 508.0 |
| F96 | 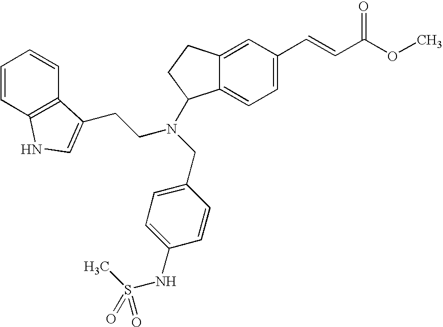 | 2.53 (A) | 544.1 |
| F97 | 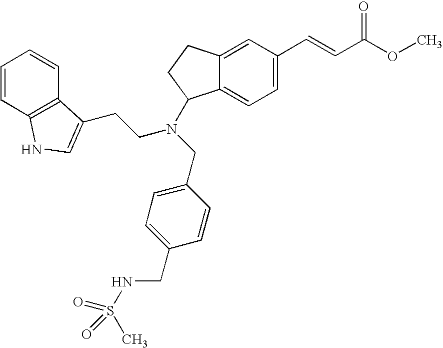 | 2.55 (A) | 558.1 |
| F98 | 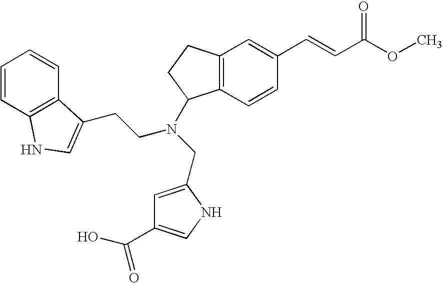 | 2.24 (A) | 484.0 |
| F99 | 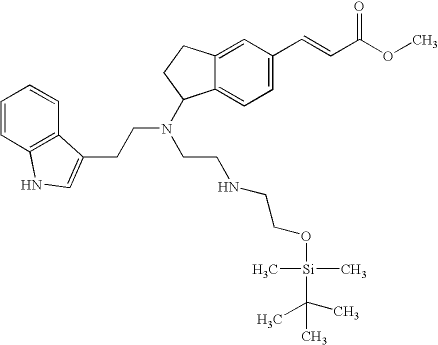 | 2.22 (A) | 562.2 |
| F100 | 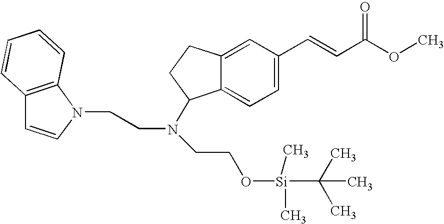 | 3.39 (A) | 519.0 |
| F101 | 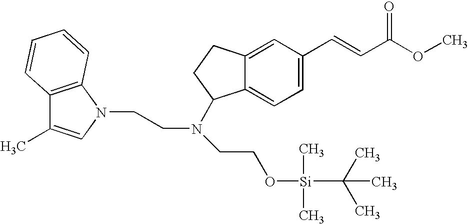 | 3.43 (A) | 533.2 |
| F102 | 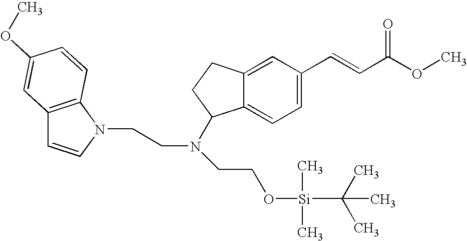 | 3.06 (A) | 549.2 |
| F103 | 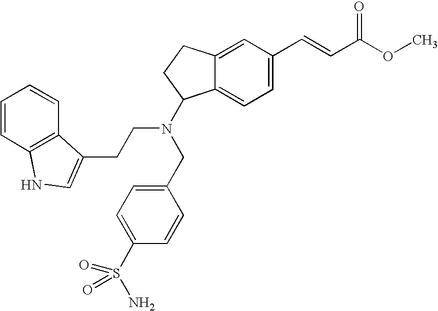 | 2.86 (A) | 530.1 |
| F104 | 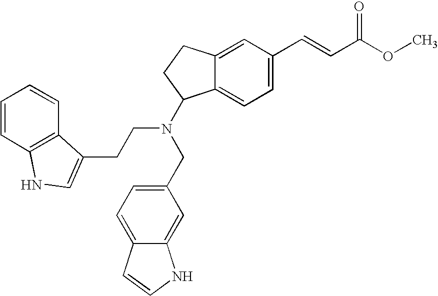 | 2.19 (A) | 490.1 |
| F105 | 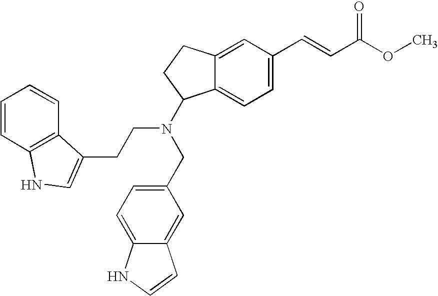 | 2.58 (A) | 490.1 |
| F106 | 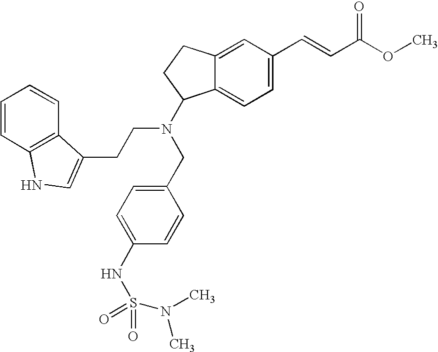 | 2.60 (A) | 573.0 |
| F107 | 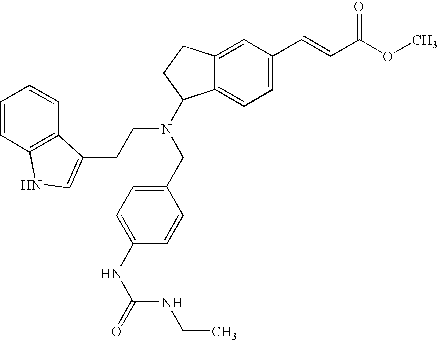 | 1.69 (A) | 537.1 |
| F108 | 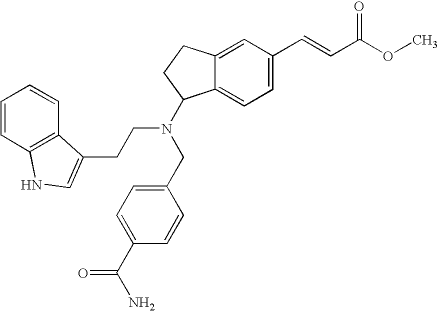 | 2.79 (A) | 494.1 |
| F109 | 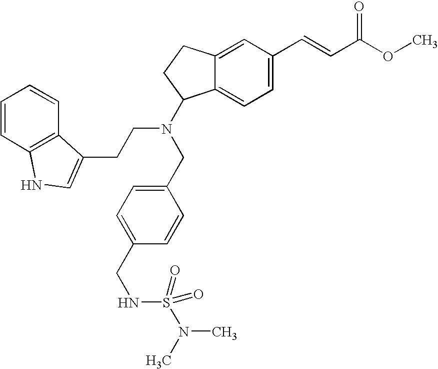 | 1.69 (A) | 587.1 |
| F110 | 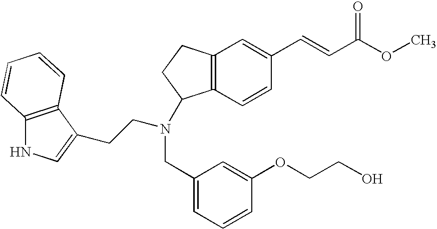 | 1.99 (A) | 511.1 |
| F111 | 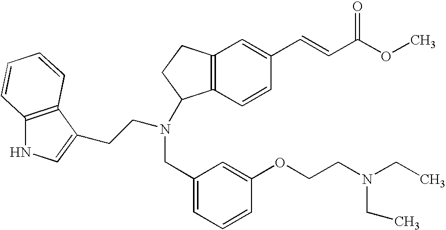 | 0.93 (A) | 566.3 |
| F112 | 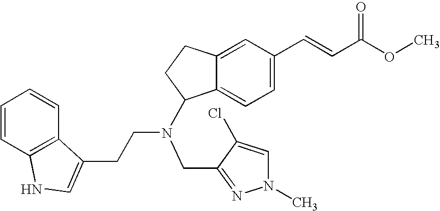 | 1.36 (A) | 489.0 |
| F113 | 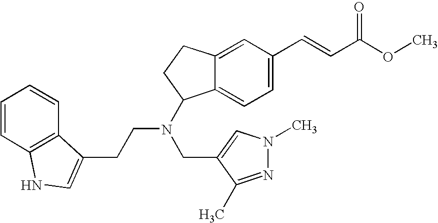 | 2.80 (A) | 469.1 |
| F114 | 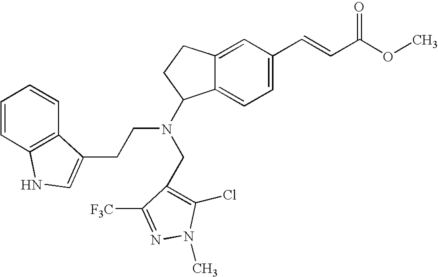 | 2.96 (A) | 556.9 |
| F115 | 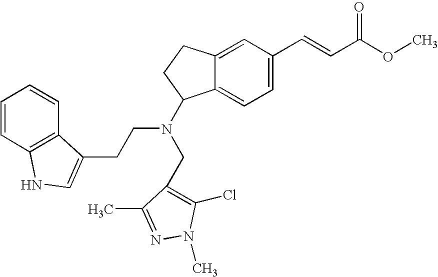 | 2.53 (A) | 502.9 |
| F116 | 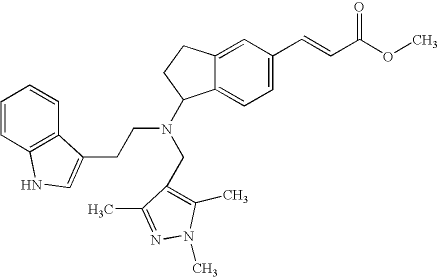 | 1.12 (A) | 483.1 |
| F117 | 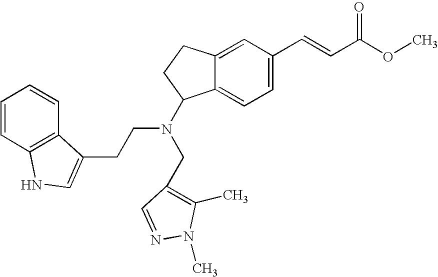 | 2.80 (A) | 469.1 |
| F118 | 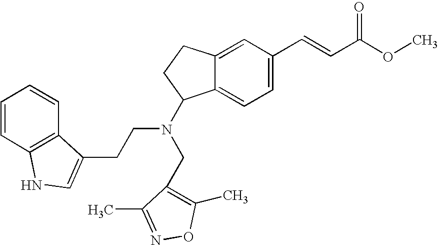 | 2.92 (A) | 470.1 |
| F119 | 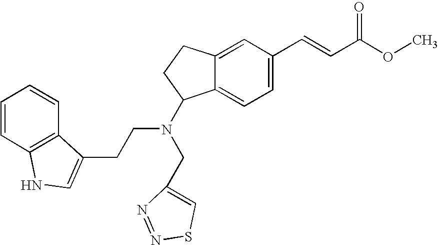 | 2.35 (A) | 458.9 |
| F120 | 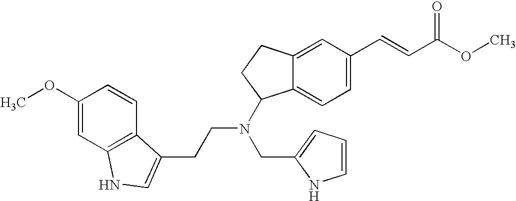 | 1.69 (A) | 469.9 |
| F121 | 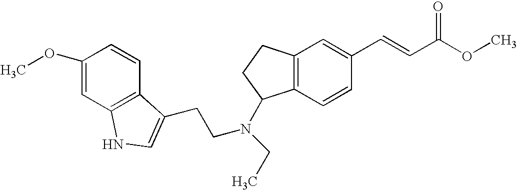 | 1.14 (A) | 419.1 |
| F122 | 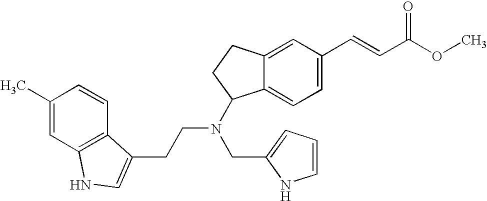 | 2.58 (A) | 453.9 |
| F123 | 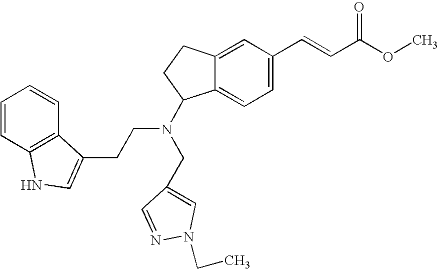 | 2.26 (A) | 469.1 |
| F124 | 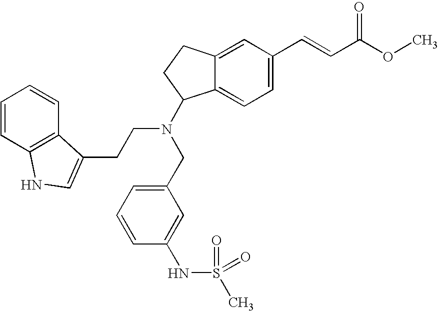 | 1.10 (A) | 544.0 |
| F125 | 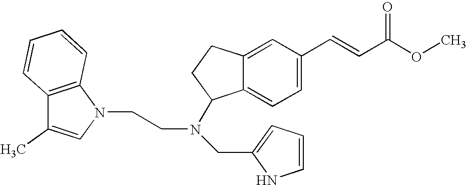 | 3.23 (A) | 454.1 |
| F126 | 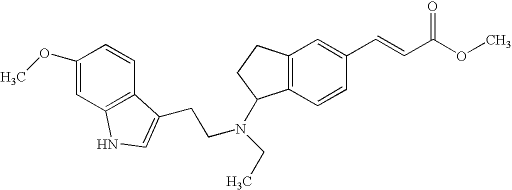 | 1.14 (A) | 419.1 |
| F127 | 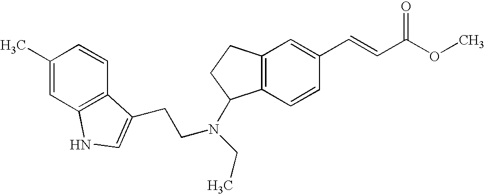 | 2.42 (A) | 403.1 |
| F128 | 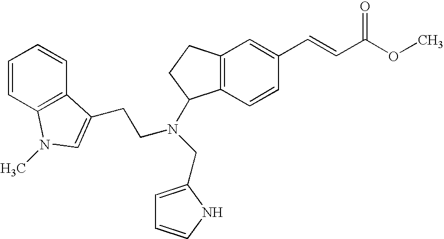 | 2.68 (A) | 453.9 |
| F129 | 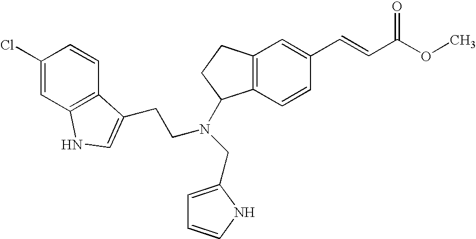 | 2.65 (A) | 473.9 |
| F130 | 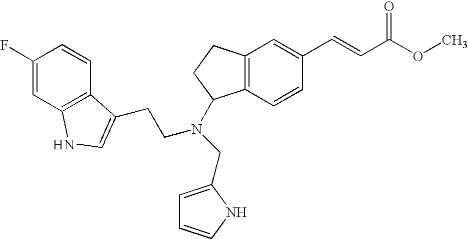 | 2.59 (A) | 457.9 |
| F131 | 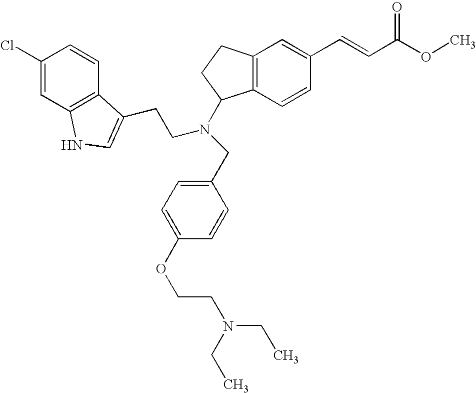 | 2.31 (A) | 600.4 |
| F132 | 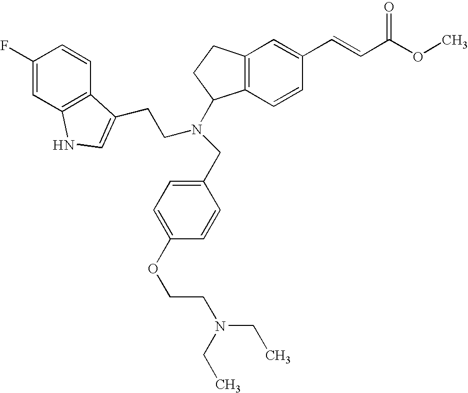 | 2.23 (A) | 584.2 |
| F133 | 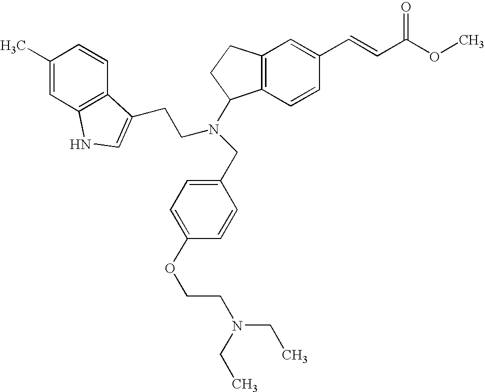 | 2.20 (A) | 580.3 |
| F134 | 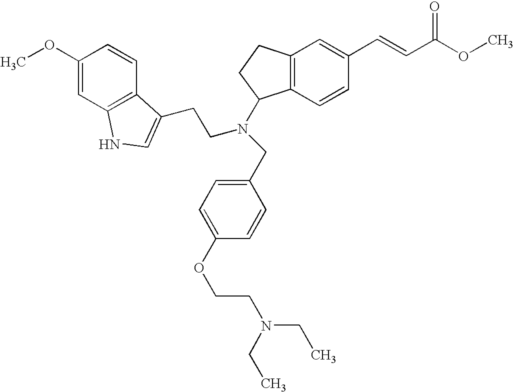 | 2.14 (A) | 596.3 |
| F135 | 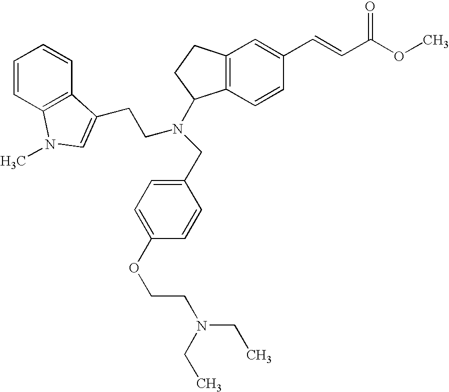 | 2.32 (A) | 580.1 |
| F136 | 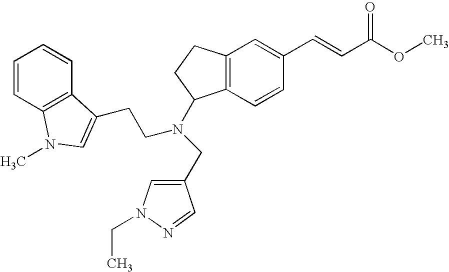 | 2.56 (A) | 483.0 |
| F137 | 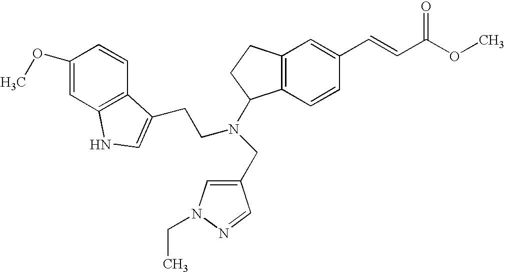 | 2.37 (A) | 499.0 |
| F138 | 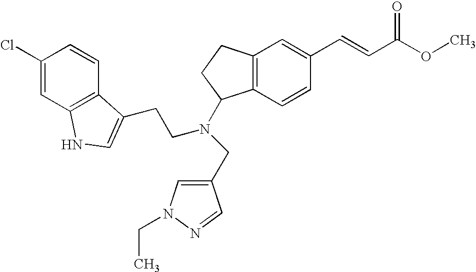 | 2.58 (A) | 503.0 |
| F139 | 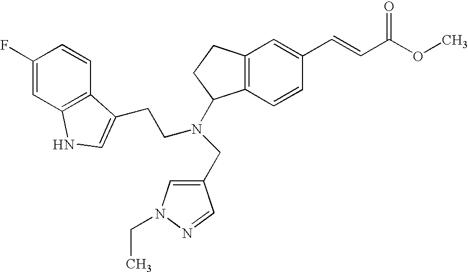 | 2.44 (A) | 487.1 |
| F140 | 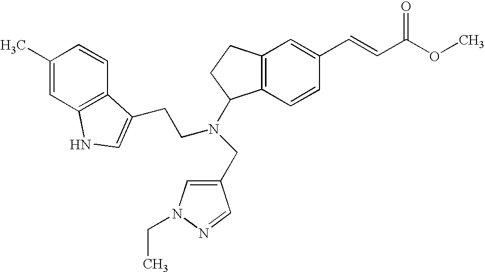 | 2.50 (A) | 483.2 |
| F141 | 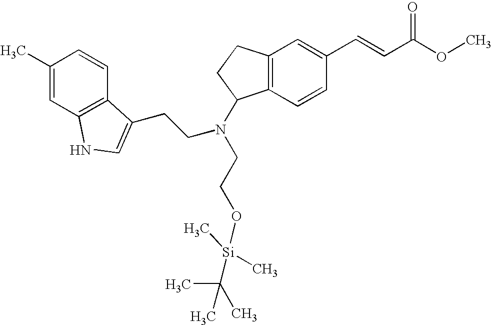 | 3.08 (A) | 533.2 |
| F142 | 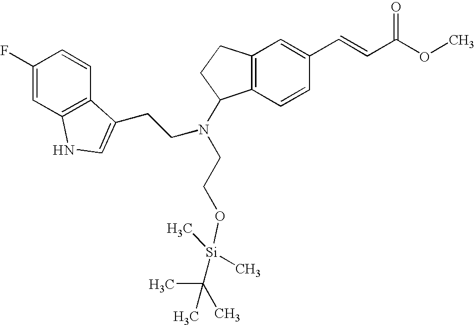 | 3.03 (A) | 537.2 |
| F143 | 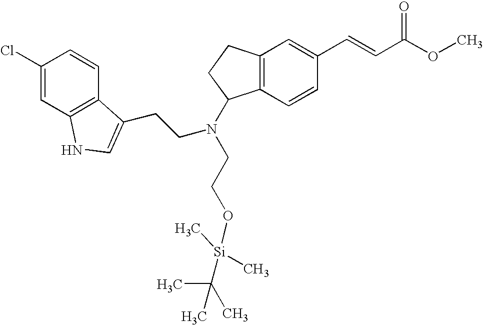 | 3.14 (A) | 553.3 |
| F144 | 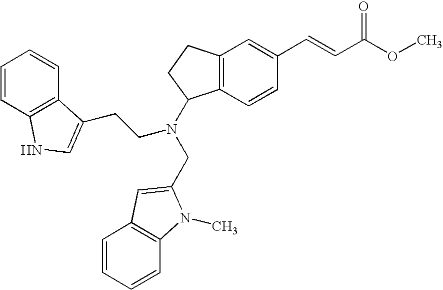 | 2.96 (A) | 504.1 |
| F145 | 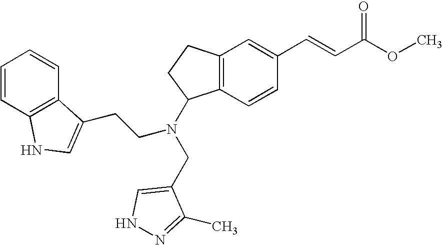 | 2.42 (A) | 455.0 |
| F146 | 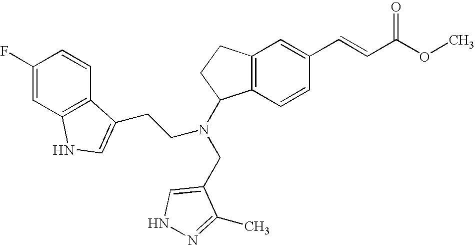 | 2.80 (A) | 473.1 |
| F147 | 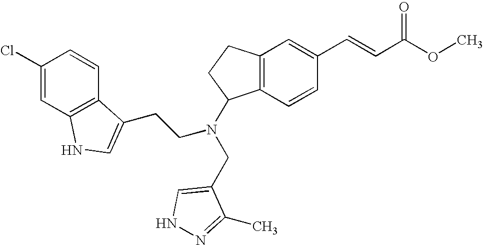 | 2.89 (A) | 489.1 |
| F148 | 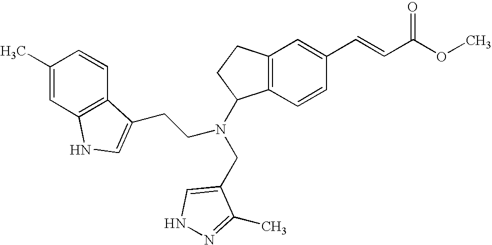 | 2.32 (A) | 469.1 |
| F149 | 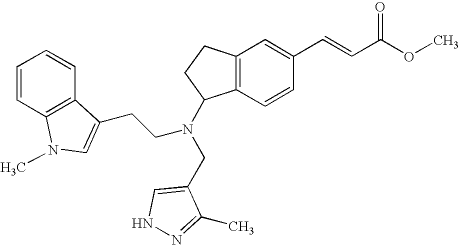 | 2.87 (A) | 469.1 |
| F150 | 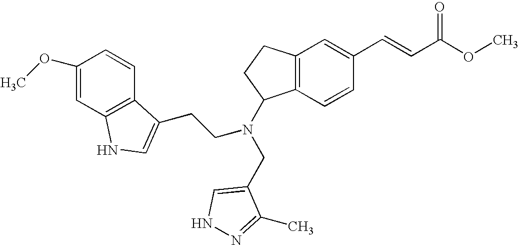 | 2.74 (A) | 485.1 |
| F151 | 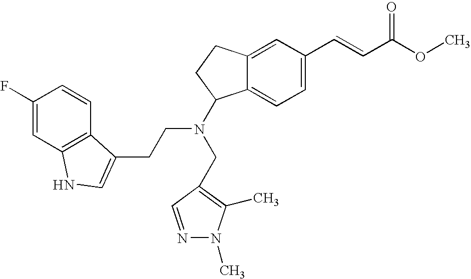 | 2.43 (A) | 487.0 |
| F152 | 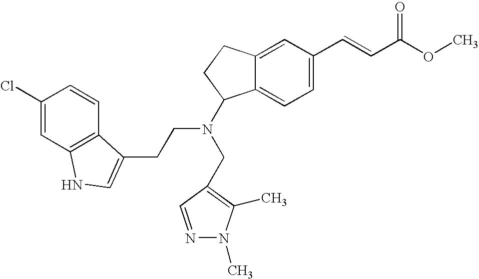 | 2.92 (A) | 503.2 |
| F153 | 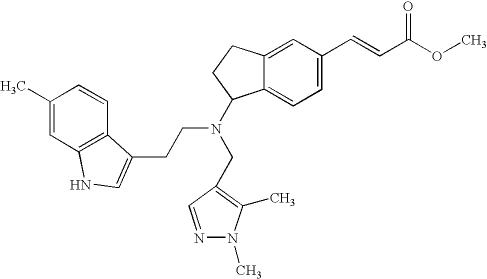 | 2.31 (A) | 483.2 |
| F154 | 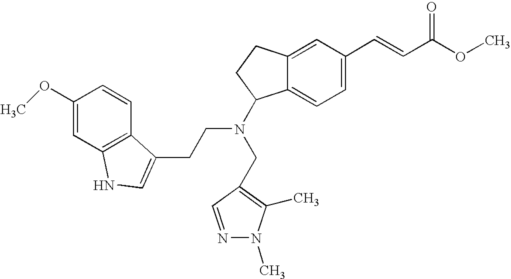 | 2.78 (A) | 499.1 |
| F155 | 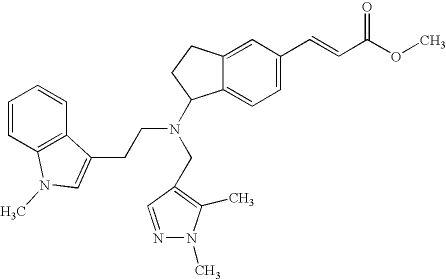 | 2.51 (A) | 483.0 |
| F156 | 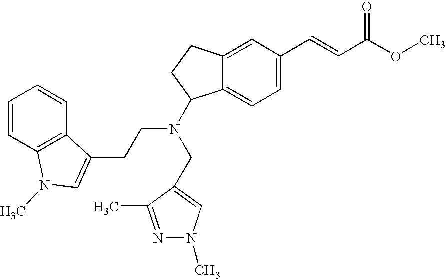 | 2.52 (A) | 483.0 |
| F157 | 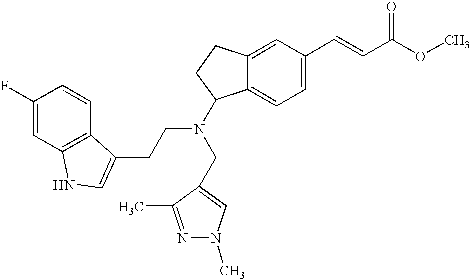 | 2.41 (A) | 487.1 |
| F158 | 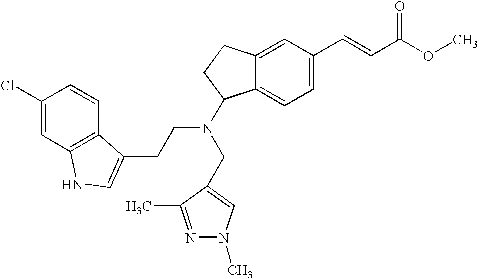 | 2.50 (A) | 502.9 |
| F159 | 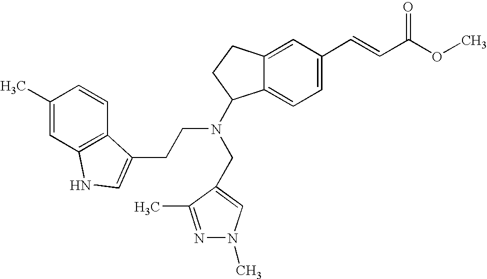 | 2.28 (A) | 483.2 |
| F160 | 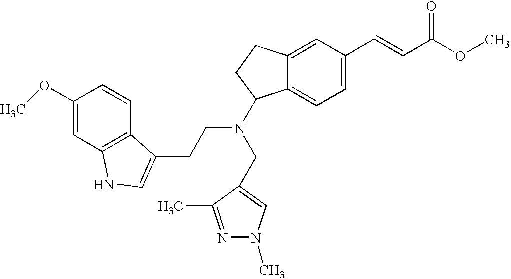 | 2.76 (A) | 499.1 |
| F161 | 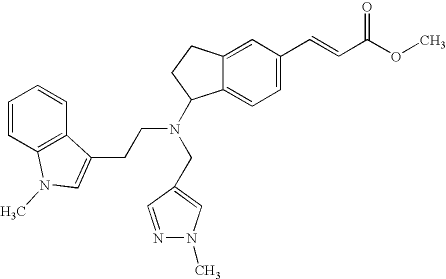 | 2.49 (A) | 469.0 |
| F162 | 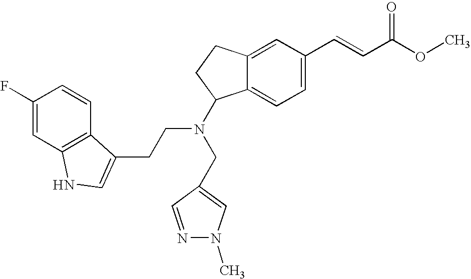 | 2.35 (A) | 473.1 |
| F163 | 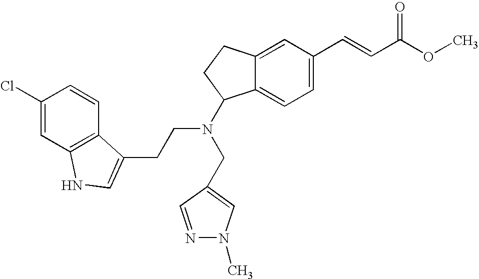 | 2.50 (A) | 490.0 |
| F164 | 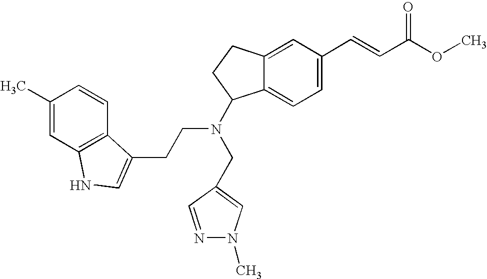 | 2.45 (A) | 469.0 |
| F165 | 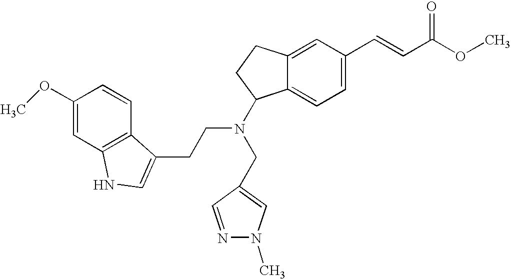 | 2.33 (A) | 485.0 |
| F166 | 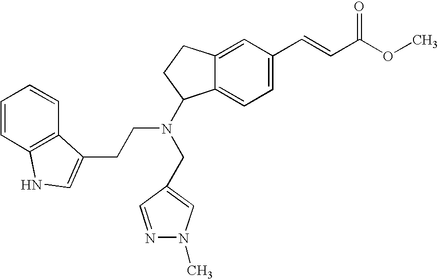 | 2.38 (A) | 455.0 |
| F167 | 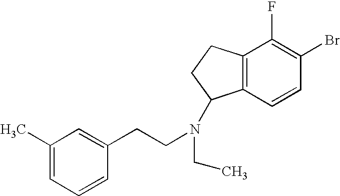 | ¹H-NMR (DMSO-d6) δ7.37 (t, 1H), 7.10 (t, 1H), 6.92 (m, 3H), 6.76 (d, 1H), 4.49 (t, 1H), 2.90 (m, 1H), 2.72 (m, 2H), 2.55 (m, 5H), 2.24 (s, 3H), 2.14 (m, 1H), 1.90 (m, 1H), 0.99 (t, 3H). | |
| F168 | 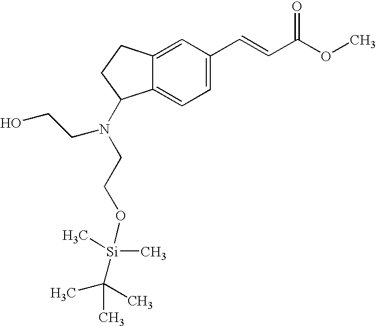 | 2.56 (A) | 420.1 |
| F169 | 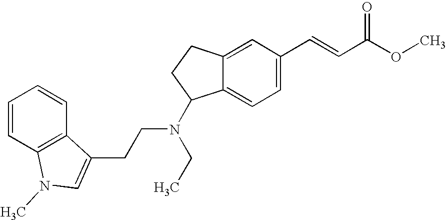 | 2.48 (A) | 403.1 |
| F170 | 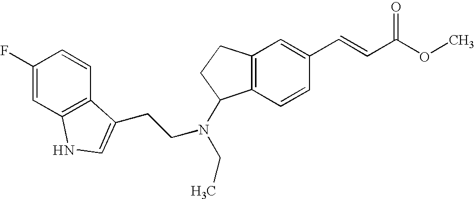 | 2.41 (A) | 407.1 |
| F171 | 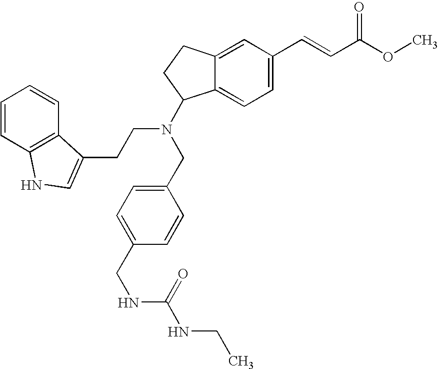 | 2.88 (A) | 551.2 |

### Intermediate G

### Methyl (2E)-3-(1-{(2-{[tert-butyl(dimethyl)silyl]oxy}ethyl)[2-(1H-indol-3-yl)ethyl]amino}-2,3-dihydro-1H-inden-5-yl)-2-propenoate

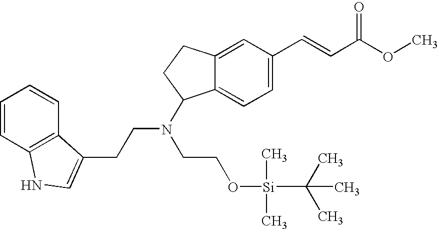

To a mixture of intermediate E (methyl (2E)-3-(1-{[2-(1H-indol-3-yl)ethyl]amino}-2,3-dihydro-1H-inden-5-yl)-2-propenoate) (0.49 g, 1.35 mmol), (2-bromoethoxy)-tert-butyldimethylsilane (0.65 g, 2.71 mmol) and N,N′-diisopropylethylamine (0.35 g, 2.71 mmol) in DMF (10 mL) was added a catalytic amount of KI. This mixture was heated at 80° C. overnight. The reaction was cooled to rt and quenched with water and extracted with CH₂Cl₂ twice. The combined organic layer was washed with water and dried over Na₂SO₄. The solvent was removed under vacuum to obtain the crude product. It was then purified with 25S Biotage eluting with 15% EtOAc in hexanes to yield methyl (2E)-3-(1-{(2-{[tert-butyl(dimethyl)silyl]oxy}ethyl)[2-(1H-indol-3-yl)ethyl]amino}-2,3-dihydro-1H-inden-5-yl)-2-propenoate as a yellow oil (0.48 g, 67%): LC/MS [M+H] 519.1, RT 2.90 min (method A); ¹H-NMR (CD₃OD) δ 7.67 (d, 1H), 7.40 (s, 1H), 7.33 (m, 4H), 7.03 (m, 2H), 6.89 (m, 1H), 6.47 (d, 1 H), 4.65 (t, 1 H), 3.76 (s, 3H), 3.63 (m, 2H), 2.90 (m, 6H), 2.64 (m, 2H), 2.24 (m, 1H), 2.02 (m, 1H), 0.85 (s, 9H), 0.00 (s, 6H).

The following compounds are synthesized in a similar manner. In the case of Intermediate G1, Intermediate A and C are used as starting materials. In the case of intermediate G5, G6, and G7, NaH is used as the base and intermediate G is used as the starting material. Intermediate G is also synthesized by a reductive amination reaction between intermediate E and {[tert-butyl(dimethyl)silyl]oxy} acetaldehyde.

| | | HPLC RT | |
|---|---|---|---|
| | | (min) | |
| Intermediate | Structure | (method) | M + H |
| G1 | 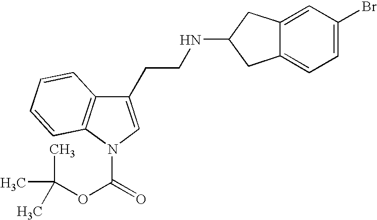 | 2.79 (A) | 455.1 |
| G2 | 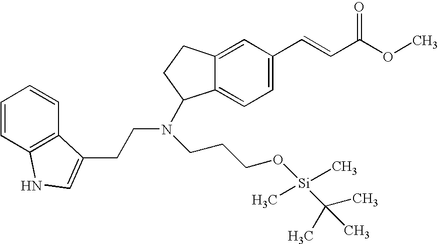 | 2.99 (A) | 533.1 |
| G3 | 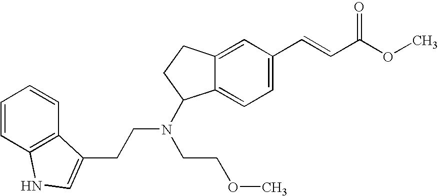 | 2.44 (A) | 419.1 |
| G4 | 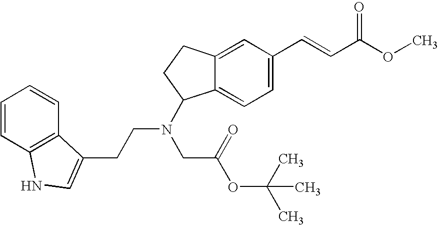 | 2.74 (A) | 475.1 |
| G5 | 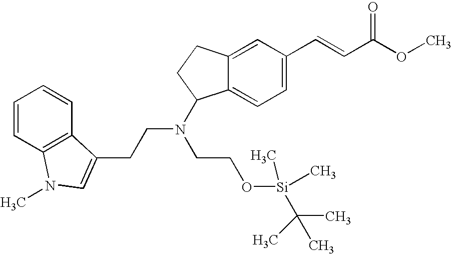 | 3.14 (A) | 533.1 |
| G6 | 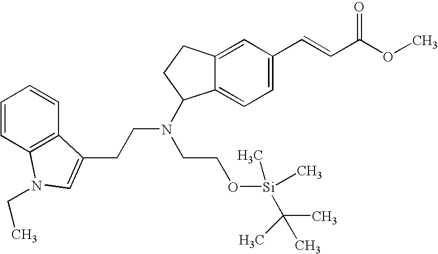 | 3.28 (A) | 547.2 |
| G7 | 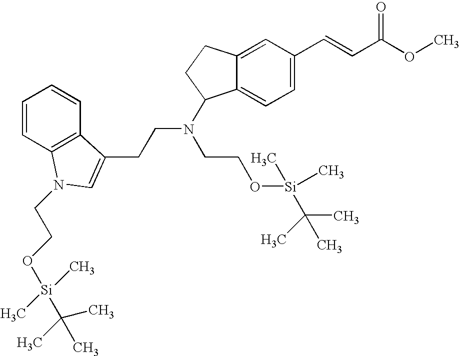 | 3.81 (A) | 677.3 |
| G8 | 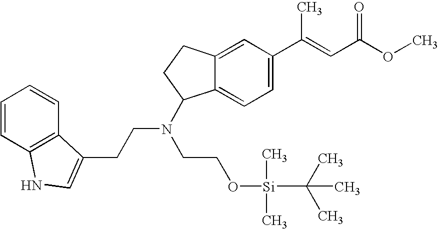 | 2.45 (B) | 533.3 |
| G9 | 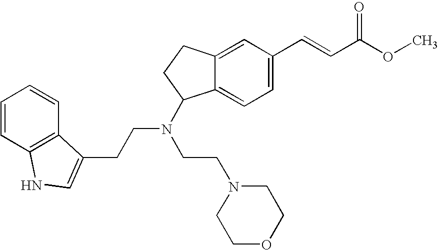 | 2.22 (A) | 474.2 |
| G10 | 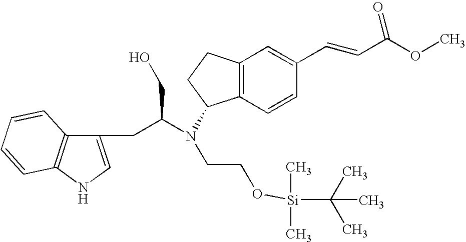 | | 549.0 |
| G11 | 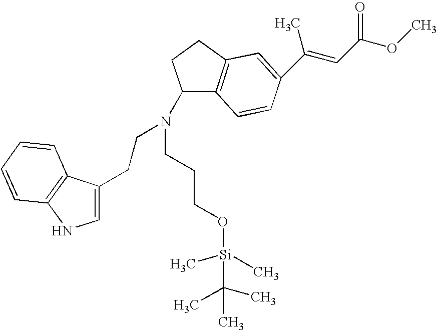 | 2.26 (B) | 547.3 |
| G12 | 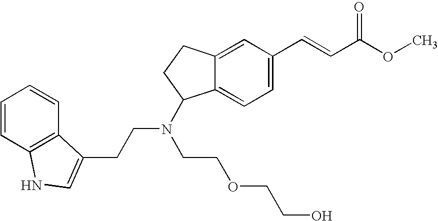 | 1.11 (A) | 449.1 |

### Intermediates H1 and H2

### Chiral separation of (±) (E)-3-(1-{(2-hydroxy-ethyl)-[2-(1H-indol-3-yl)-ethyl]-amino}-indan-5-yl)-acrylic acid methyl ester

Intermediate H1 Intermediate H2

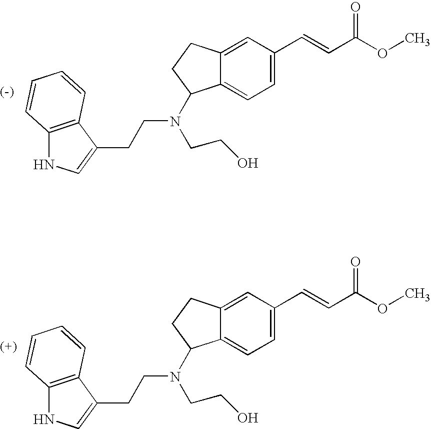

Racemic Intermediate J (methyl (2E)-3-(1-{(2-hydroxyethyl)[2-(1H-indol-3-yl)ethyl]amino}-2,3-dihydro-1H-inden-5-yl)-2-propenoate) (0.31 g) was separated with ChiralPAK AD-H using 25-40% iPrOH in hexane with 0.1% Et₃N (flow rate=15 mL/min, 250 uL/injection) to obtain first peak (RT=18.73 min, 105 mg): [α]_{D} (MeOH)=+70.2 (c 1.0). Second peak (RT=22.93 min, 103 mg): [α]_{D} (MeOH)=−67.9 (c 1.0). The overall recovery yield was 67%.

Intermediates I1 and I2

### Chiral separation of (±) (E)-3-{1-[2-(1H-indol-3-yl)-ethylamino]-indan-5-yl}-acrylic acid methyl ester

Intermediate I1 Intermediate I2

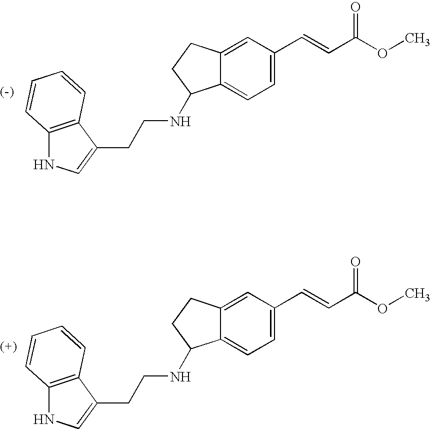

Racemic Intermediate E (methyl (2E)-3-(1-{[2-(1H-indol-3-yl)ethyl]amino}-2,3-dihydro-1H-inden-5-yl)-2-propenoate) (0.28 g) was separated with ChiralPAK AD-H using 20-28% MeOH/EtOH (1/1) in hexane with 0.1% Et₃N (flow rate=15 mL/min, 250 uL/injection) to obtain the first peak (RT=6.70 min, 100 mg) and the second peak (RT=8.10 min, 85 mg). The overall recovery yield was 66%.

### Intermediate J

### Methyl (2E)-3-(1-{(2-hydroxyethyl)[2-(1H-indol-3-yl)ethyl]amino}-2,3-dihydro-1H-inden-5-yl)-2-propenoate

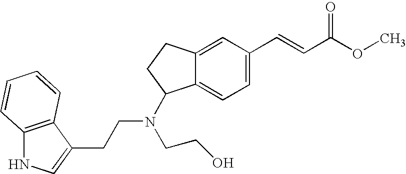

To a solution of Intermediate G (methyl (2E)-3-(1-{(2-{[tert-butyl(dimethyl)silyl]oxy}ethyl)[2-(1H-indol-3-yl)ethyl]amino}-2,3-dihydro-1H-inden-5-yl)-2-propenoate) (1.74 g, 3.35 mmol) in MeOH (5 mL) was added 5% TFA in water (15 mL). The mixture was stirred at 40° C. for 3 h. The reaction was quenched with saturated NaHCO₃ and extracted with EtOAc twice. The combined organic layer was washed with brine, dried over Na₂SO₄. The solvent was removed under reduced pressure to give the crude residue. It was purified with 25 M Biotage eluting with 100% EtOAc to obtain desired product methyl (2E)-3-(1-{(2-hydroxyethyl)[2-(1H-indol-3-yl)ethyl]amino}-2,3-dihydro-1H-inden-5-yl)-2-propenoate as a yellow oil (1.10 g, 80%): LC/MS [M+H] 405.0, RT 2.26 min (method A). ¹H-NMR (CD₃OD) δ 7.68 (d,1H), 7.43 (s, 1H), 7.30 (m, 4H), 7.03 (m, 2H), 6.90 (m, 1 H), 6.50 (d, 1H), 4.66 (t, 1H), 3.79 (s, 3H), 3.61 (m, 2H), 2.92 (m, 8H), 2.28 (m, 1H), 2.06 (m, 1H).

The following compounds are synthesized in a similar manner:

| | | HPLC RT | |
|---|---|---|---|
| | | (min) | |
| Intermediate | Structure | (method) | M + H |
| J1 | 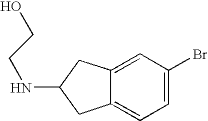 | 0.52 (A) | 256.0 |
| J2 | 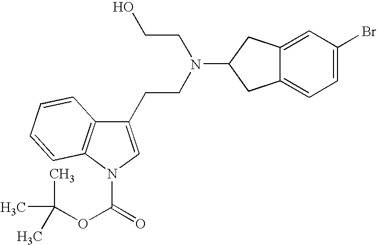 | 3.19 (B) | 499.5 |
| J3 | 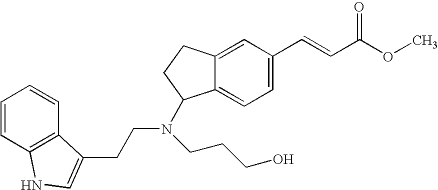 | 2.17 (A) | 419.0 |
| J4 | 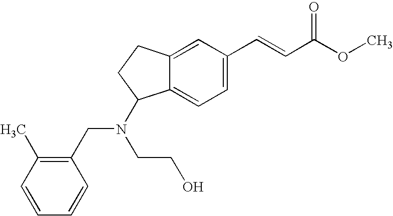 | 2.16 (A) | 366.0 |
| J5 | 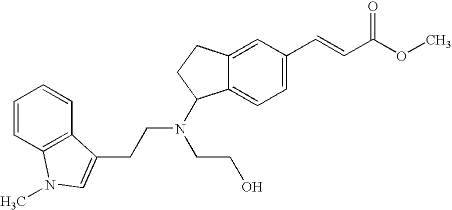 | 2.84 (A) | 419.1 |
| J6 | 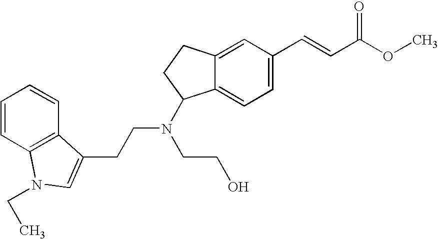 | 2.42 (A) | 433.1 |
| J7 | 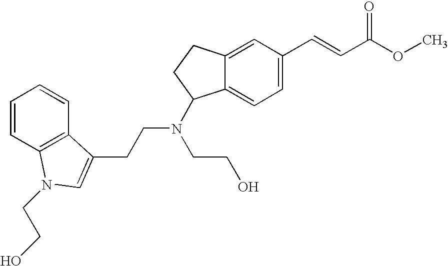 | 2.70 (A) | 449.1 |
| J8 | 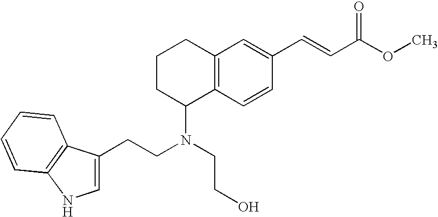 | 2.23 (A) | 419.0 |
| J9 | 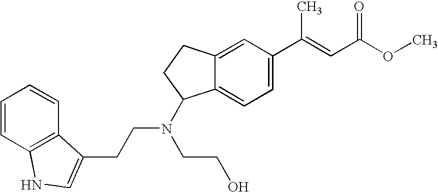 | 2.81 (A) | 419.2 |
| J10 | 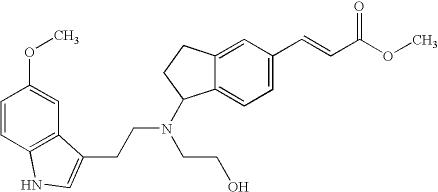 | 2.10 (A) | 435.1 |
| J11 | 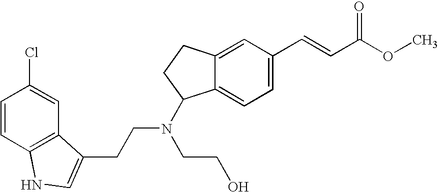 | 2.30 (A) | 439.1 |
| J12 | 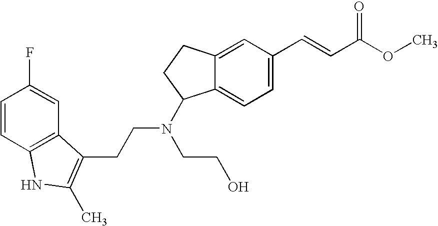 | 2.29 (A) | 437.0 |
| J13 | 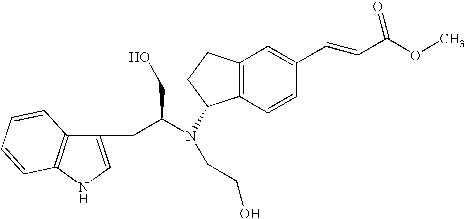 | 2.09 (A) | 435.0 |
| J14 | 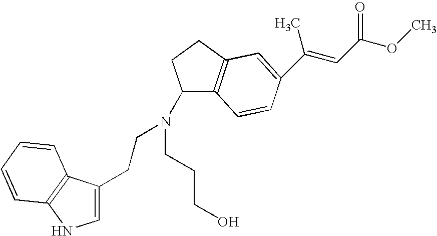 | 2.81 (A) | 433.1 |
| J15 | 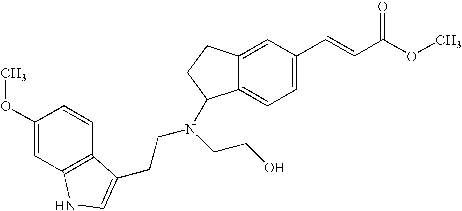 | 2.14 (A) | 435.1 |
| J16 | 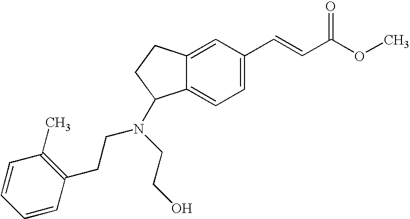 | 2.44 (A) | 380.2 |
| J17 | 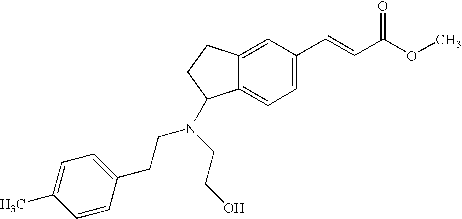 | 2.46 (A) | 380.2 |
| J18 | 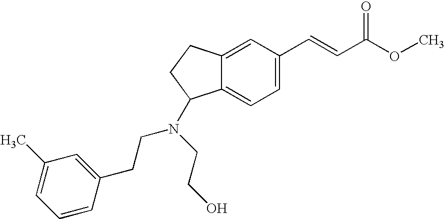 | 2.45 (A) | 380.2 |
| J19 | 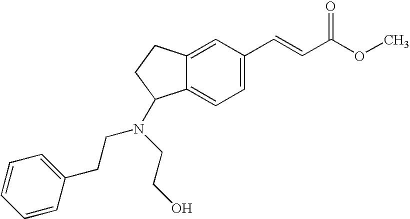 | 2.28 (A) | 366.1 |
| J20 | 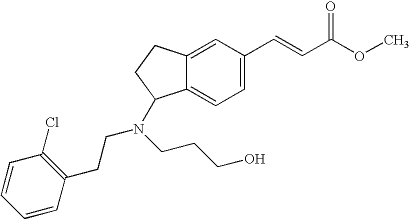 | 2.24 (A) | 414.2 |
| J21 | 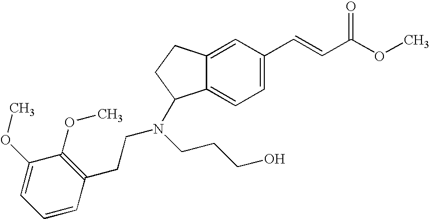 | 2.19 (A) | 440.1 |
| J22 | 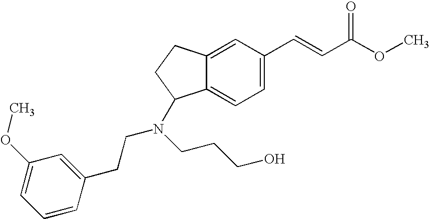 | 2.17 (A) | 410.1 |
| J23 | 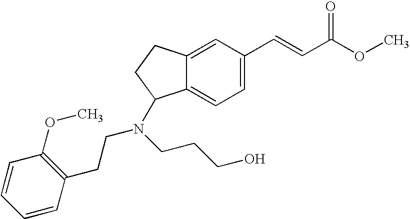 | 2.23 (A) | 410.1 |
| J24 | 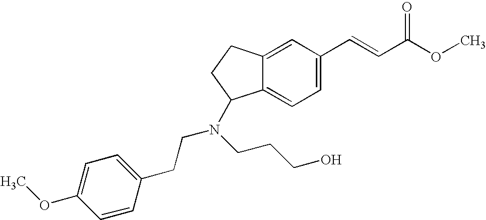 | 2.16 (A) | 410.1 |
| J25 | 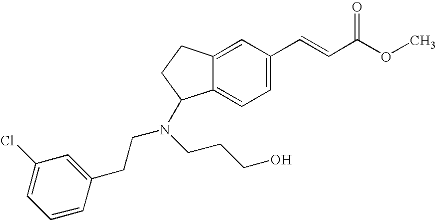 | 2.28 (A) | 414.2 |
| J26 | 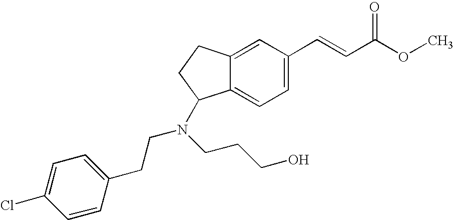 | 2.30 (A) | 414.2 |
| J27 | 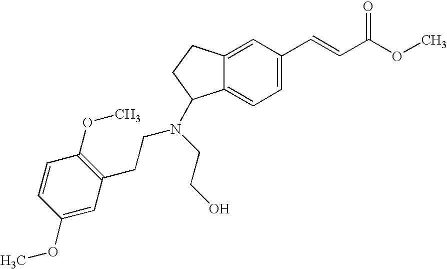 | 2.20 (A) | 426.0 |
| J28 | 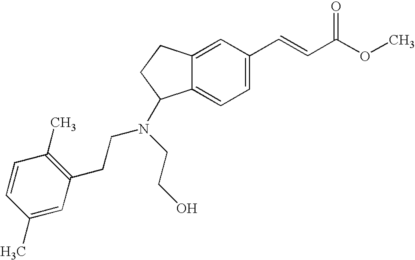 | 2.35 (A) | 394.1 |
| J29 | 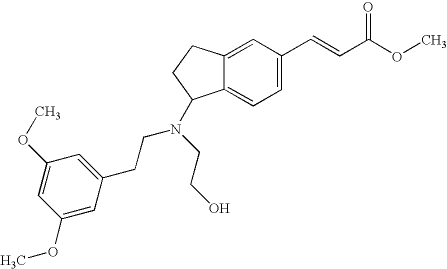 | 2.19 (A) | 426.0 |
| J30 | 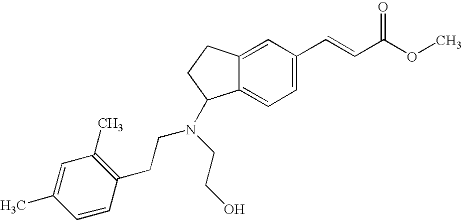 | 2.36 (A) | 394.0 |
| J31 | 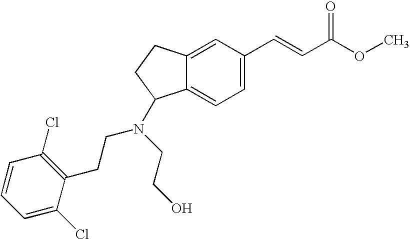 | 2.30 (A) | 434.0 |
| J32 | 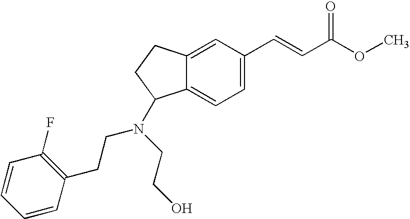 | 2.16 (A) | 384.0 |
| J33 | 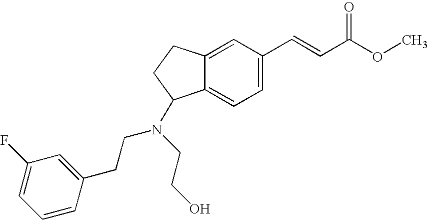 | 2.17 (A) | 384.0 |
| J34 | 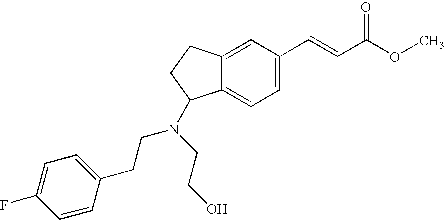 | 2.18 (A) | 384.0 |
| J35 | 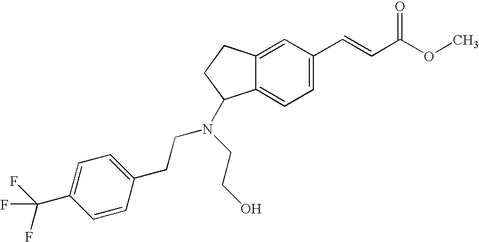 | 2.38 (A) | 434.0 |
| J36 | 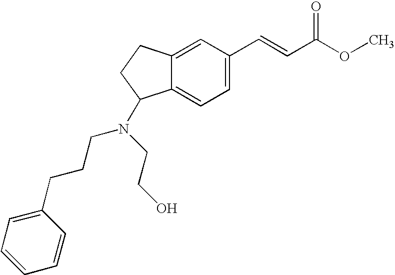 | 2.24 (A) | 380.0 |
| J37 | 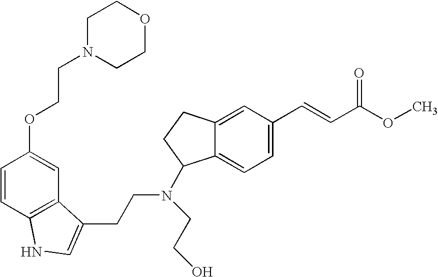 | 1.68 (A) | 534.1 |
| J38 | 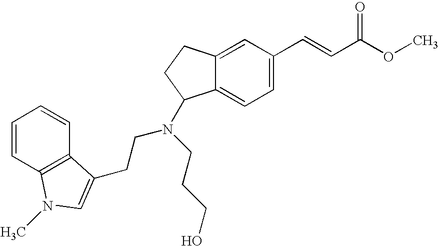 | 2.36 (A) | 433.2 |
| J40 | 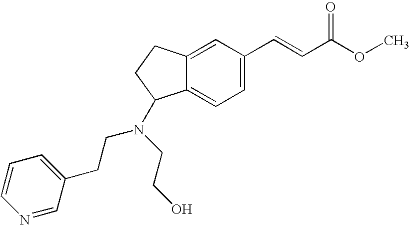 | 0.98 (A) | 366.7 |
| J41 | 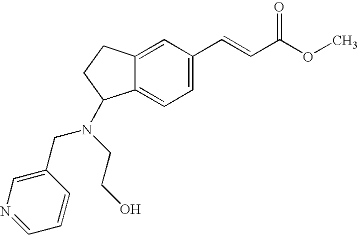 | 1.56 (A) | 352.8 |
| J42 | 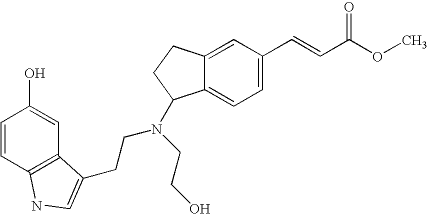 | 1.96 (A) | 421.1 |
| J43 | 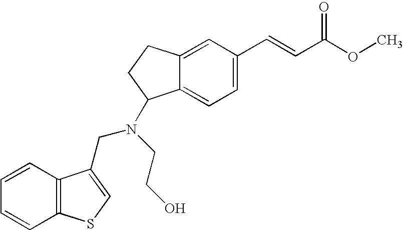 | 2.46 (A) | 407.9 |
| J44 | 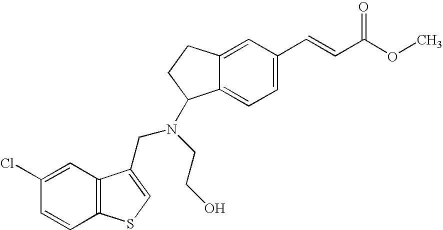 | 2.60 (A) | 441.9 |
| J45 | 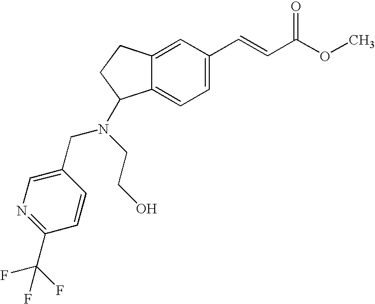 | TLC R_{f} = 0.14 (EtOAc:hexanes, 3:7) | |
| J46 | 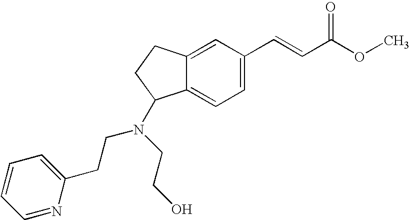 | 1.99 (A) | 367.0 |
| J47 | 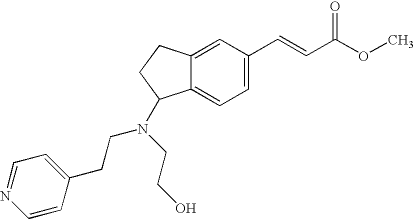 | 1.18 (A) | 367.0 |
| J48 | 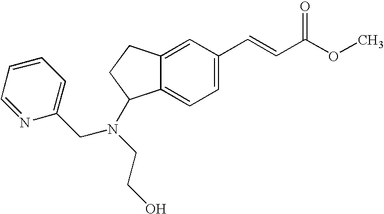 | 2.03 (A) | 353.0 |
| J49 | 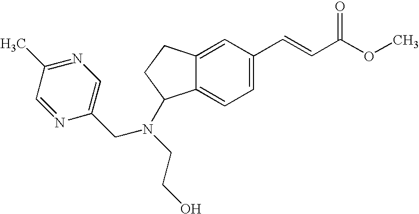 | 1.38 (A) | 368.1 |
| J50 | 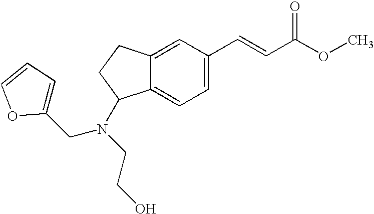 | 1.84 (A) | 341.9 |
| J51 | 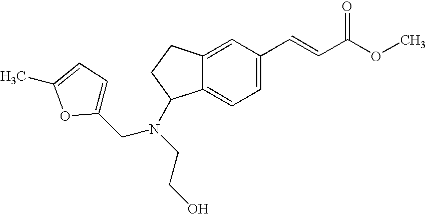 | 2.01 (A) | 356.0 |
| J52 | 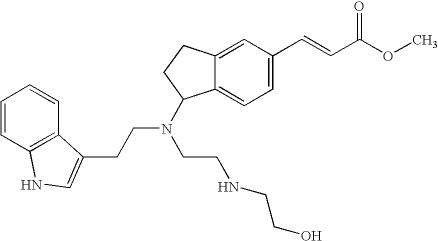 | 2.18 (A) | 448.2 |
| J53 | 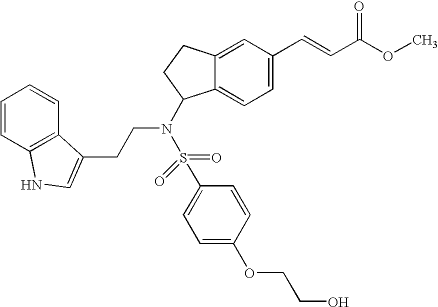 | 3.32 (A) | 561.0 |
| J54 | 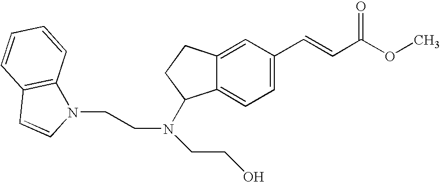 | 1.70 (A) | 404.9 |
| J55 | 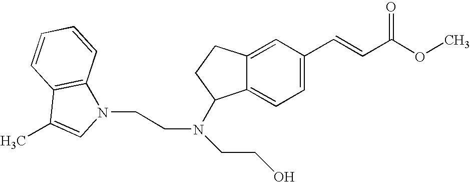 | 2.51 (A) | 419.0 |
| J56 | 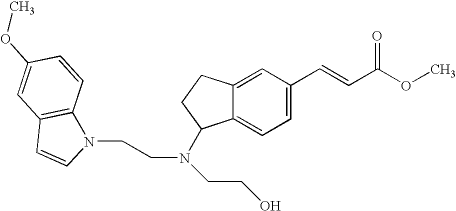 | 1.32 (A) | 435.0 |
| J57 | 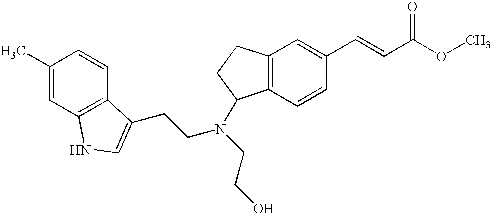 | 2.34 (A) | 419.1 |
| J58 | 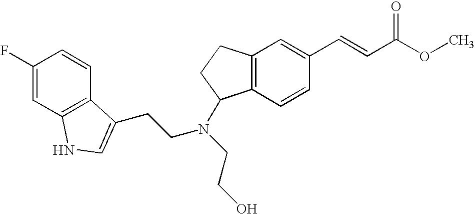 | 2.30 (A) | 423.0 |
| J59 | 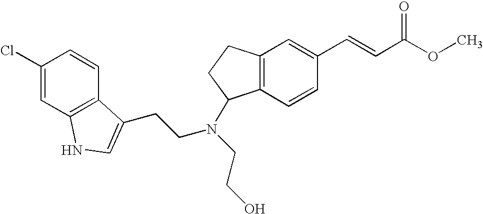 | 2.41 (A) | 438.9 |

### Intermediate K

### 5-Oxo-5,6,7,8-tetrahydro-2-naphthalenyl trifluoromethanesulfonate

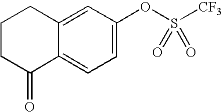

6-Hydroxy-1-tetralone (1.00 g, 6.17 mmol), and Et₃N (1.72 mL, 12.33 mmol) were dissolved in CH₂Cl₂ (30 mL) at rt. The resulting solution was cooled to 0° C. at which time trifluoromethanesulfonic anhydride (1.56 g, 9.25 mmol) was added dropwise. The reaction was stirred for 20 min at which time a solution of saturated NaHCO₃ was added. The bi-phasic mixture was vigorously stirred for 10 min then diluted with CH₂Cl₂. The organic layer was collected and washed with 1N HCl and brine. The organic phase was collected, dried over Na₂SO₄, and filtered. After removal of the solvent under vacuum, the crude oil was purified by silica gel chromatography using 20% EtOAc in Hex as eluent give the 5-oxo-5,6,7,8-tetrahydro-2-naphthalenyl trifluoromethanesulfonate as a near colorless oil (1.32 g, 73% yield): ¹H NMR (CD₂Cl₂) δ 8.10 (m, 1H), 7.23 (m, 2H), 3.04 (dd, 2H), 2.68 (dd, 2H), 2.19 (m, 2H).

### Intermediate L

### Methyl (2E)3-(5-oxo-5,6,7,8-tetrahydro-2-naphthalenyl)-2-propenoate

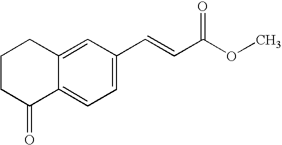

Intermediate K (5-Oxo-5,6,7,8-tetrahydro-2-naphthalenyl trifluoromethanesulfonate) (1.3 g, 4.42 mmol), methyl acrylate (3.98 mL, 44.2 mmol), Et₃N (1.23 mL, 8.84 mmol), and DPPP (100 mg, 0.24 mmol) were dissolved in DMF (20 mL) at rt. After the solution was degassed with nitrogen for 15 min., Pd(OAc)₂ (50 mg, 0.22 mol) was added and the solution was heated to 80° C. for 16 h. The reaction was cooled to rt and the volatile solvents were removed under vacuum. The crude product was purified by silica gel chromatography using 20-30% EtOAc in Hex as eluent to give methyl (2E)-3-(5oxo-5,6,7,8-tetrahydro-2-naphthalenyl)-2-propenoate (670 mg, 66% yield) as a white solid: LC/MS [M+H] 231.1, RT 3.22 min (method A); ¹H NMR (CD₂Cl₂) δ 7.99 (d, 1H), 7.67 (d, 1H), 7.49 (m, 1H), 7.4 (s, 1H), 6.54 (d, 1H), 3.81 (s, 3H), 3.88 (dd, 2H), 2.66 (dd, 2H), 2.16 (m, 2H).

### Intermediate M

### Methyl (2E)-3-{1-{acetyl[2-(1H-indol-3-yl)ethyl]amino}-2,3-dihydro-1H-inden-5-yl)-2-propenoate

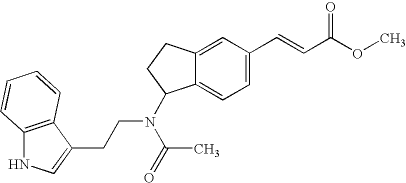

To a solution of Intermediate E (methyl (2E)-3-(1-{[2-(1H-indol-3-yl)ethyl]amino}-2,3-dihydro-1H-inden-5-yl)-2-propenoate) (82 mg, 0.23 mmol) in THF at 0° C. was added acetyl chloride (21 mg, 0.27 mmol) and Et₃N (34 mg, 0.34 mmol). The mixture was stirred at rt overnight. In the morning the reaction was quenched with water. The mixture was extracted with CH₂Cl₂ twice and the combined organic layer was washed with brine and dried over Na₂SO₄. It was concentrated under vacuo to obtain the crude residue. It was then purified with silica gel column chromatography eluting with 80% EtOAc in Hex to give the desired product as a pair of rotomers (74 mg, 81%): LC/MS [M+H] 402.9, RT 2.98 min (method A); ¹H-NMR (CD₃OD) δ 7.73 (d, J=12 Hz, 1 H), 7.56 (s, 1 H), 7.50 (m, 1H), 7.28 (m, 2H), 7.10 (m, 4H), 6.57 (dd, J=12 Hz, 3 Hz, 1H), 5.96 and 5.57 (t, 1H), 3.79 (s, 3H), 3.48 (t, 1H), 3.05 (m, 5H), 2.50 (m, 1H), 2.20 (s, 3H), 2.16 (m, 1H).

The following compounds are synthesized in a similar manner:

| | | HPLC RT | |
|---|---|---|---|
| | | (min) | |
| Intermediate | Structure | (method) | M + H |
| M1 | 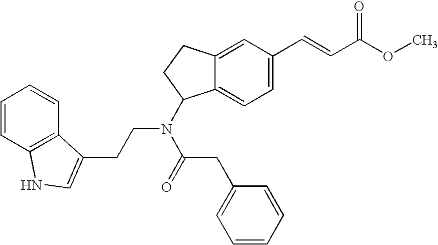 | 3.96 (A) | 479.1 |
| M2 | 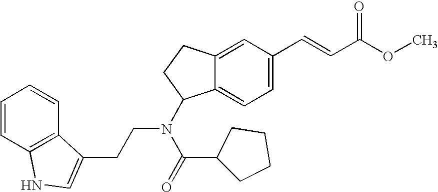 | 3.57 (A) | 457.1 |
| M3 | 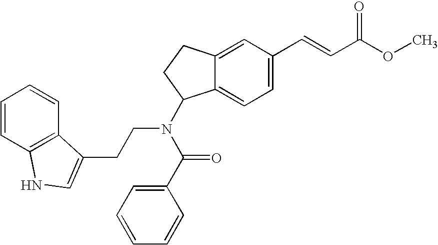 | 3.58 (A) | 465.0 |
| M4 | 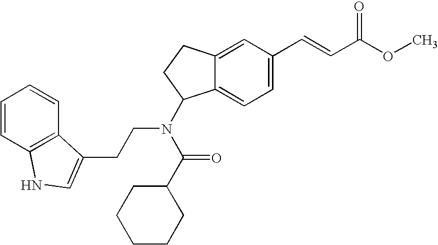 | 3.66 (A) | 471.1 |
| M5 | 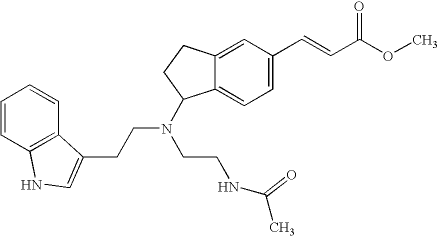 | 2.30 (A) | 446.1 |

### Intermediate N

### methyl (2E)-3-(1-{[(ethylamino)carbonyl][2-(1H-indol-3-yl)ethyl]amino}-2,3-dihydro-1H-inden-5-yl)acrylate

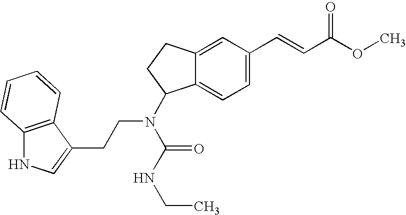

To a solution of Intermediate E [methyl (2E)-3-(1-{[2-(1H-indol-3-yl)ethyl]amino}-2,3-dihydro-1H-inden-5-yl)-2-propenoate] (88 mg, 0.24 mmol) in CH₂Cl₂ (2 mL) at 0° C. was added ethyl isocyanate (19 mg, 0.27 mmol). The resulting mixture was stirred at rt for 2 hrs. The mixture was then concentrated under vacuum and purified with 25S Biotage eluting with 60% EtOAc in Hex to obtain the desired product methyl (2E)-3-(1-{[(ethylamino)carbonyl][2-(1H-indol-3-yl)ethyl]amino}-2,3-dihydro-1H-inden-5-yl)acrylate as a yellow solid (100 mg, 95%): LC/MS [M+H] 432.0, RT 3.10 min (method A).

The following compounds are synthesized in a similar manner:

| | | HPLC RT | |
|---|---|---|---|
| | | (min) | |
| Intermediate | Structure | (method) | M + H |
| N1 | 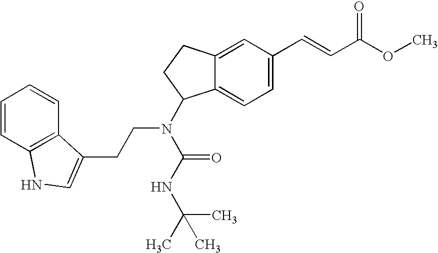 | 3.54 (A) | 460.2 |
| N2 | 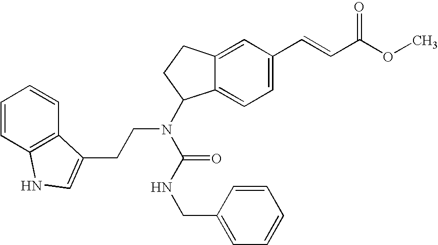 | 3.43 (A) | 493.9 |
| N3 | 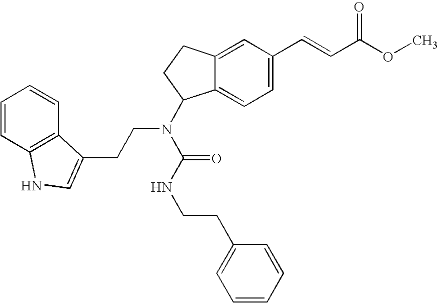 | 3.65 (A) | 508.0 |
| N4 | 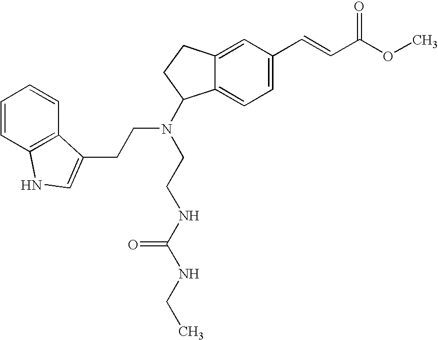 | 2.41 (A) | 475.1 |
| N5 | 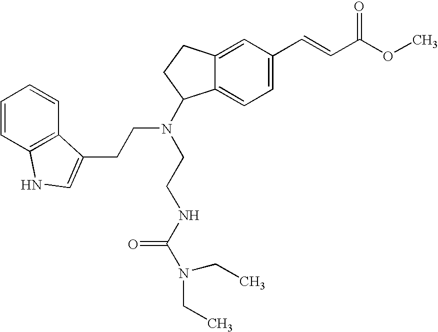 | 1.35 (A) | 503.1 |
| N6 | 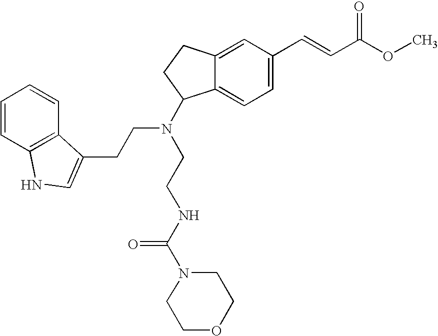 | 1.41 (A) | 517.1 |

### Intermediate O

### Methyl (2E)-3-{1-[[2-(1H-indol-3-yl)ethyl](phenylsulfonyl)amino]-2,3-dihydro-1H-inden-5-yl}acrylate

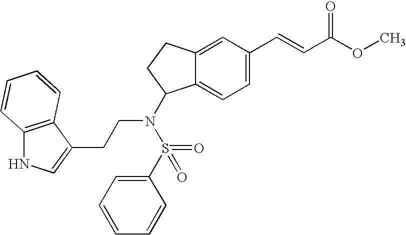

To a solution of Intermediate E [methyl (2E)-3-(1-{[2-1H-indol-3-yl)ethyl]amino}-2,3-dihydro-1H-inden-5-yl)-2-propenoate] (100 mg, 0.28 mmol) in CH₂Cl₂ (2 mL) at 0° C. added benzenesulfonyl chloride (58 mg, 0.33 mmol) and Et₃N (42 mg, 0.42 mmol) dropwise. The resulting mixture was stirred at rt overnight before it was concentrated under vacuum. The resulting residue was purified with 25S Biotage eluting with 25% EtOAc in Hex to obtain the desired product methyl (2E)-3-{1-[[2-(1H-indol-3-yl)ethyl](phenylsulfonyl)amino]-2,3-dihydro-1H-inden-5-yl}acrylate as a yellow solid ( 67 mg, 48%): LC/MS [M+H] 500.9, RT 3.82 min (method A).

The following compounds are synthesized in a similar manner:

| | | HPLC RT | |
|---|---|---|---|
| | | (min) | |
| Intermediate | Structure | (method) | M + H |
| O1 | 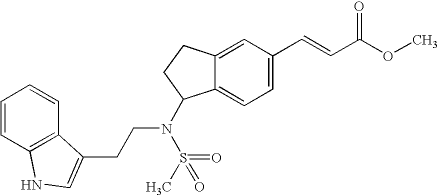 | 3.34 (A) | 439.0 |
| O2 | 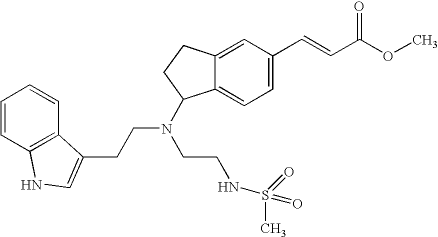 | 2.39 (A) | 482.0 |
| O3 | 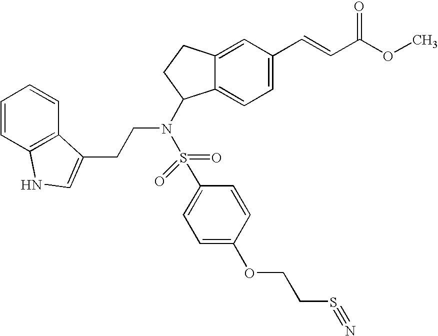 | 3.58 (A) | 569.8 |
| O4 | 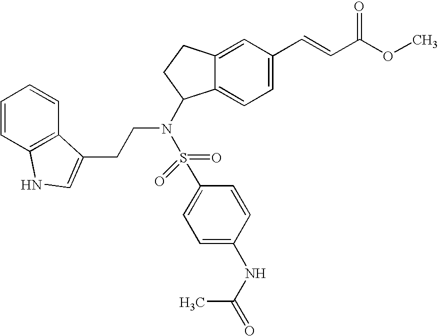 | 3.36 (A) | 558.1 |
| O5 | 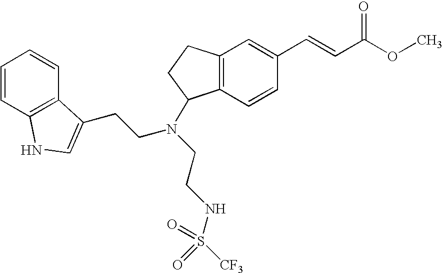 | 2.68 (A) | 536.0 |

### Intermediates P1 and P2

### Chiral separation of racemic methyl (2E)-3-(1-f(3-hydroxypropyl)f2- H-indol-3-yl)ethyl]amino]-2.3-dihydro-1H-inden-5-yl)acrylate

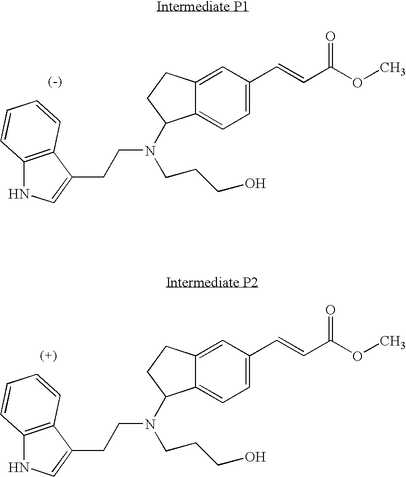

Racemic Intermediate J3 methyl (2E)-3-(1-{(3-hydroxypropyl)[2-(1H-indol-3-yl)ethyl]amino}-2,3-dihydro-1H-inden-5-yl)acrylate (0.6 g) was separated with ChiralPAK AD-H using 35-65% B (B=1:1 methanol: ethanol) in hexane with 0.1% Et₃N (flow rate=15 mUmin, 150 uL/injection) to obtain first peak (RT=5.35 min, 95 mg). Second peak (RT=7.98 min, 85 mg): [α]_{D}=+90.2 (c 1.0, MeOH). The overall recovery yield was 41%.

### Intermediate Q

### Methyl (2E)-3-{1-[(pyridin-3-ylmethyl)amino]-2,3-dihydro-1H-inden-5-yl}acrylate

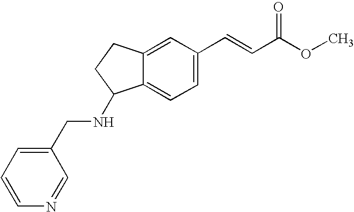

Intermediate D [methyl (2E)-3-(1-oxo-2,3-dihydro-1H-inden-5-yl)-2-propenoate] (250 mg, 1.16 mmol) was dissolved in CH₂Cl₂, treated with Titanium(IV)methoxide (497 mg, 2.89 mmol, 2.5 eq), molecular sieves (4A, 370 mg), and 3-(methylamine)pyridine (137 mg, 1.27 mmol, 1.1 eq). The reaction mixture was stirred at rt under nitrogen overnight. In the next morning, the mixture was treated with sodium triacetoxyborohydride (610 mg, 2.89 mmol, 2.5 eq) and stirred at rt overnight. It was then diluted with CH₂Cl₂ and MeOH and the reaction was quenched with water and stirred for 30 min. Celite was then added to the emulsion and filtered. The filtrate was then absorbed on silica and purified on the Biotage with 2-3% MeOH in CH₂Cl₂ to afford 340 mg (95% yield) of the product as a solid. R_{f}=0.25 (silica, MeOH: CH₂Cl₂, 4:96); LC/MS=308.9 [(M+H)+, RT=1.24 min (method A)]; ¹H NMR (DMSO-d6) δ 1.73-1.86 (m, 1H), 2.26-2.37 (m, 1H), 2.67-2.78 (m, 1H), 2.88-2.97 (m, 1H), 3.70 (s, 3H), 3.76 (d, 1H), 3.82 (d, 1H), 4.13 (t, 1H), 6.58 (d,1H), 7.31-7.35 (m, 1H), 7.41 (d, 1H), 7.52 (d, 1H), 7.57 (s, 1H), 7.64 (d, 1H), 7.77-7.82 (m, 1H), 8.42 (dd, 1H), 8.57 (d, 1H).

Alternatively, 1 N aqueous HCl is used to quench the reaction without the dilution with CH₂Cl₂ and MeOH. The desired product is collected in its hydrochloride salt form. Intermediate E, Q13, Q54, Q58, Q59, and Q60 are prepared as their hydrochloride salts.

The following compounds are synthesized in a similar manner:

| | | HPLC RT | |
|---|---|---|---|
| | | (min) | |
| Intermediate | Structure | (method) | M + H |
| Q1 | 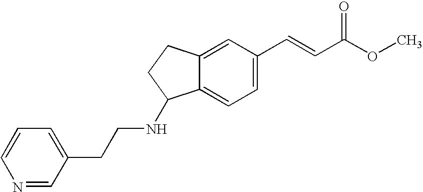 | 1.04 (A) | 323.0 |
| Q2 | 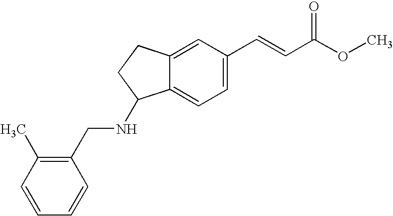 | 2.29 (A) | 321.9 |
| Q3 | 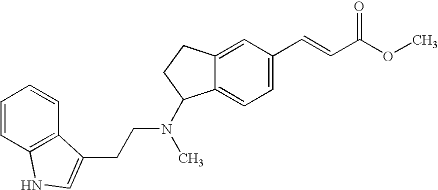 | 2.36 (A) | 375.0 |
| Q4 | 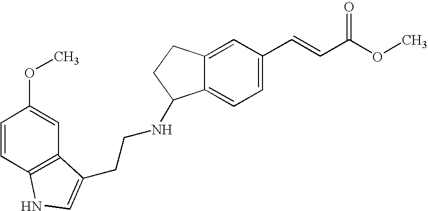 | 2.20 (A) | 391.0 |
| Q5 | 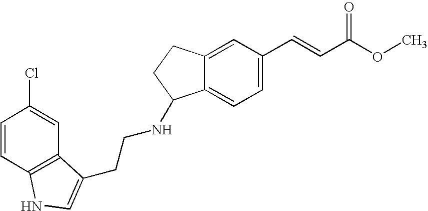 | 2.94 (A) | 395.1 |
| Q6 | 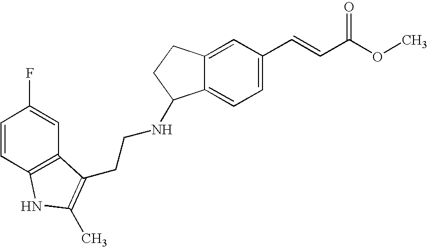 | 2.87 (A) | 393.0 |
| Q7 | 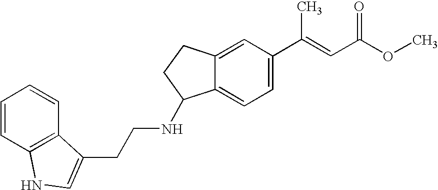 | 2.04 (B) | 375.2 |
| Q8 | 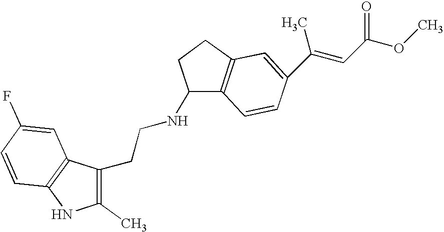 | 2.57 (A) | 407.0 |
| Q9 | 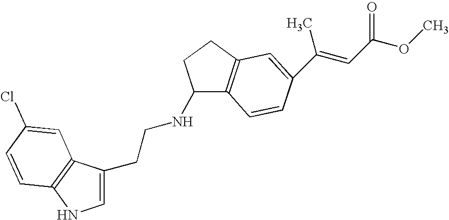 | 2.63 (A) | 409.2 |
| Q10 | 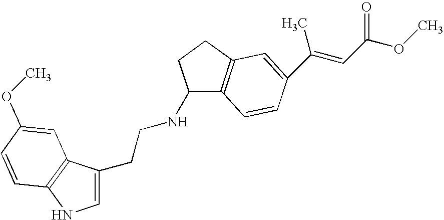 | 1.93 (A) | 405.2 |
| Q11 | 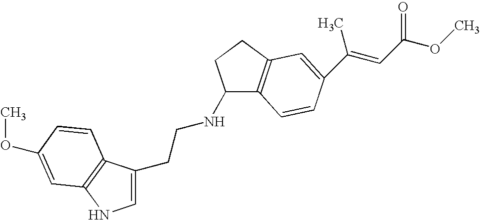 | 2.46 (A) | 405.2 |
| Q12 | 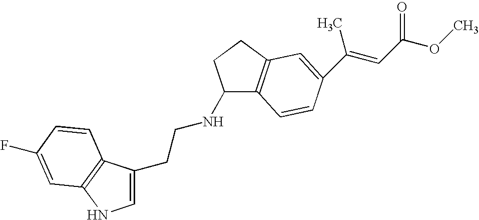 | 2.52 (A) | 393.2 |
| Q13 | 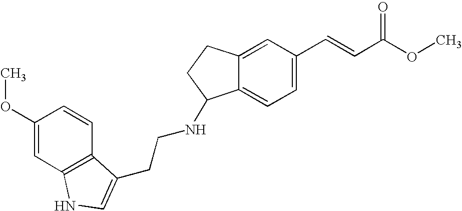 | 2.24 (A) | 391.0 |
| Q14 | 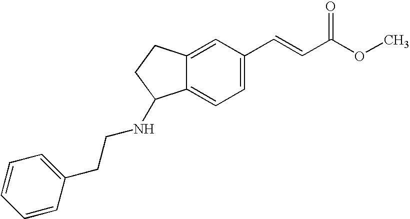 | 2.18 (A) | 322.1 |
| Q15 | 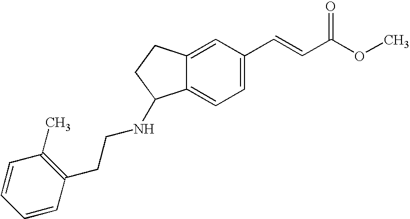 | 2.26 (A) | 336.1 |
| Q16 | 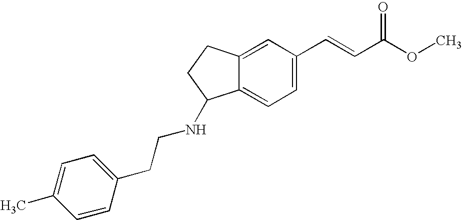 | 2.30 (A) | 336.1 |
| Q17 | 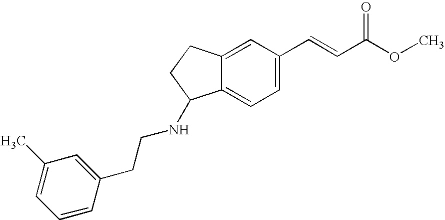 | 2.29 (A) | 336.1 |
| Q18 | 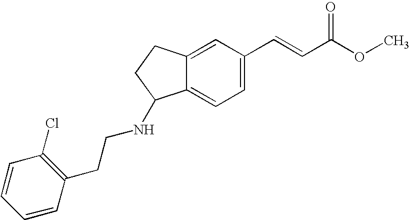 | 2.28 (A) | 356.1 |
| Q19 | 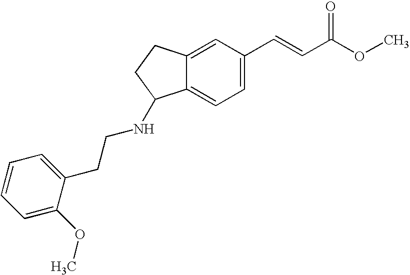 | 2.24 (A) | 352.1 |
| Q20 | 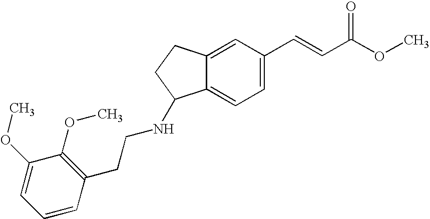 | 2.21 (A) | 382.1 |
| Q21 | 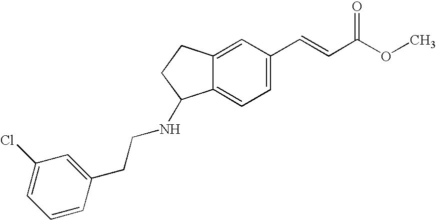 | 2.32 (A) | 356.1 |
| Q22 | 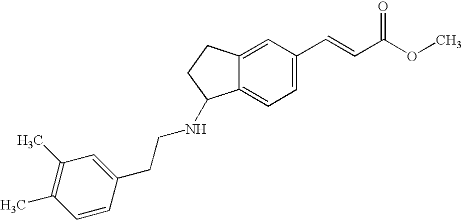 | 2.39 (A) | 350.1 |
| Q23 | 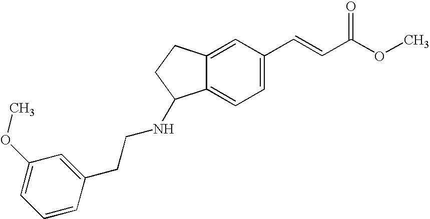 | 2.19 (A) | 352.1 |
| Q24 | 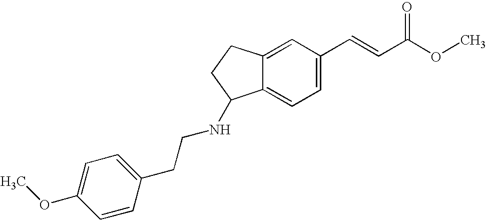 | 2.18 (A) | 352.1 |
| Q25 | 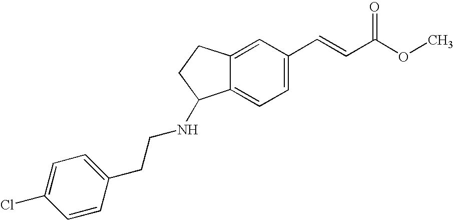 | 2.32 (A) | 356.0 |
| Q26 | 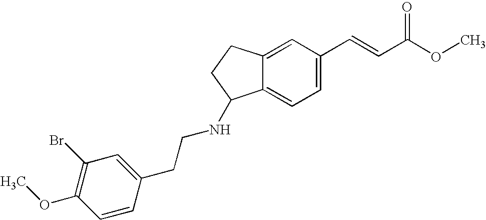 | 2.34 (A) | 429.9 |
| Q27 | 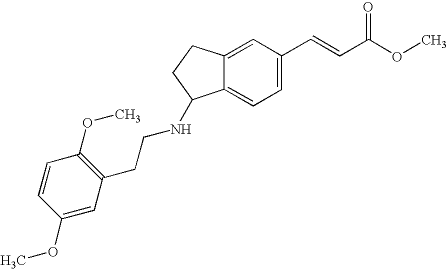 | 2.25 (A) | 382.0 |
| Q28 | 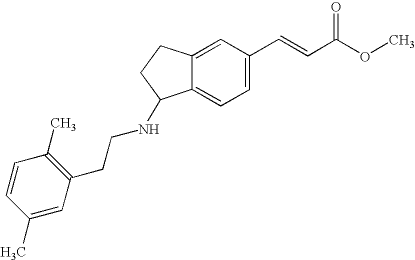 | 2.39 (A) | 350.0 |
| Q29 | 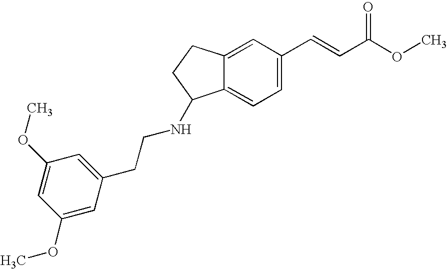 | 2.23 (A) | 382.0 |
| Q30 | 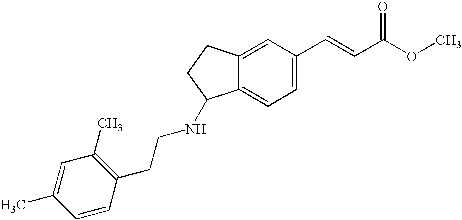 | 2.38 (A) | 350.0 |
| Q31 | 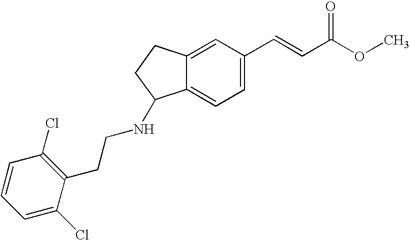 | 2.32 (A) | 390.0 |
| Q32 | 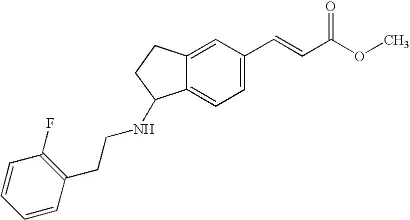 | 2.17 (A) | 339.9 |
| Q33 | 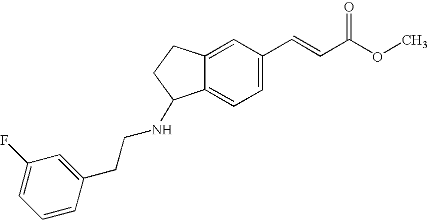 | 2.20 (A) | 339.9 |
| Q34 | 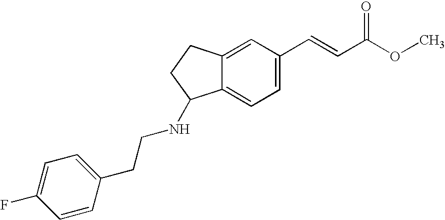 | 2.20 (A) | 339.9 |
| Q35 | 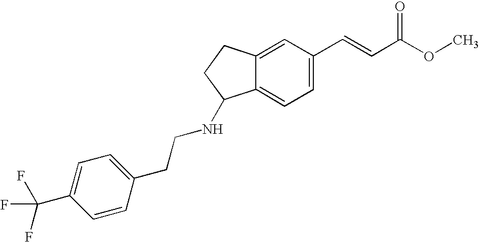 | 2.40 (A) | 389.9 |
| Q36 | 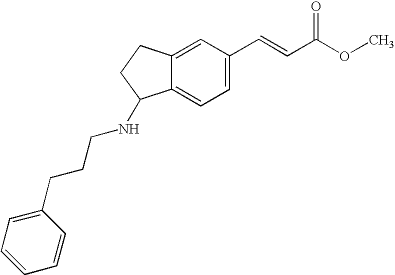 | 1.96 (A) | 336.0 |
| Q37 | 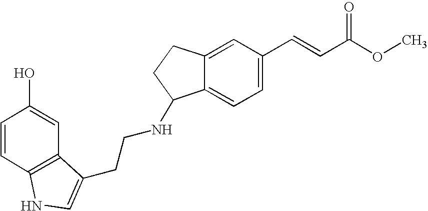 | 2.09 (A) | 377.0 |
| Q38 | 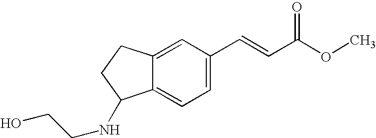 | 1.25 (A) | 261.9 |
| Q40 | 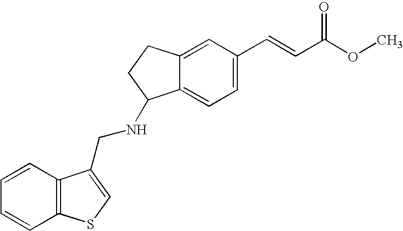 | 2.32 (A) | 363.6 |
| Q41 | 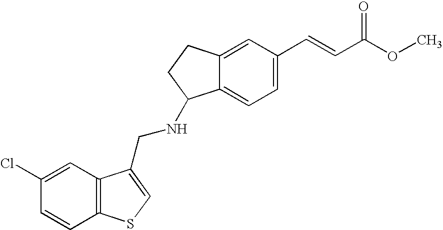 | 2.59 (A) | 397.8 |
| Q42 | 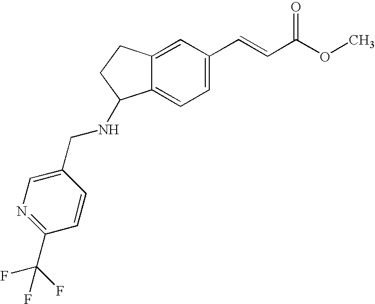 | 2.05 (A) | 376.3 |
| Q43 | 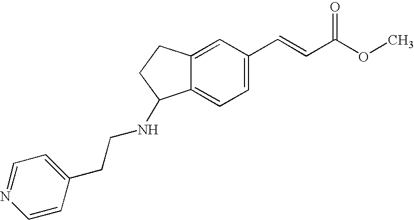 | 1.07 (A) | 323.0 |
| Q44 | 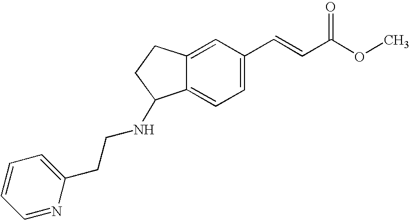 | 1.74 (A) | 323.1 |
| Q45 | 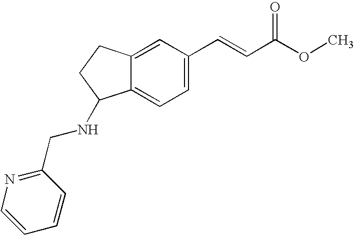 | 2.00 (A) | 309.1 |
| Q46 | 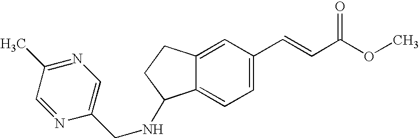 | 1.88 (A) | 323.9 |
| Q47 | 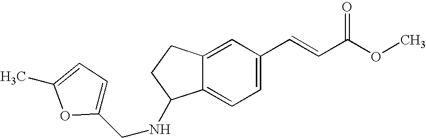 | 2.56 (A) | 311.9 |
| Q48 | 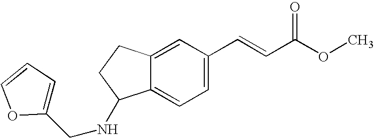 | 2.26 (A) | 297.9 |
| Q49 | 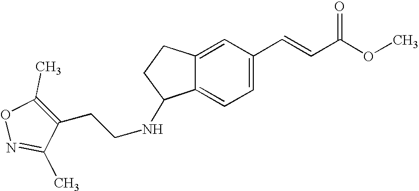 | 0.47 (A) | 341.0 |
| Q50 | 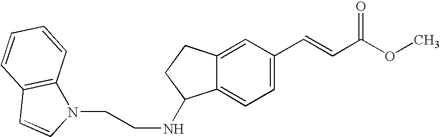 | 2.23 (A) | 361.0 |
| Q51 | 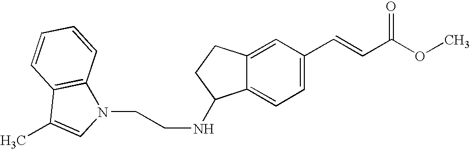 | 2.39 (A) | 375.0 |
| Q52 | 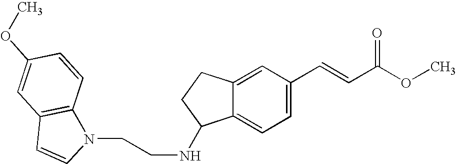 | 1.32 (A) | 391.0 |
| Q53 | 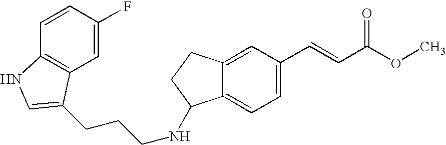 | 2.88 (A) | 393.0 |
| Q54 | 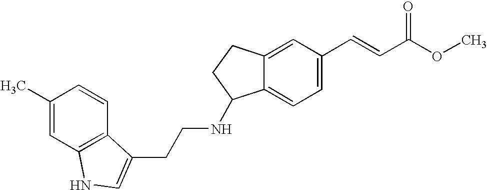 | 2.32 (A) | 375.0 |
| Q55 | 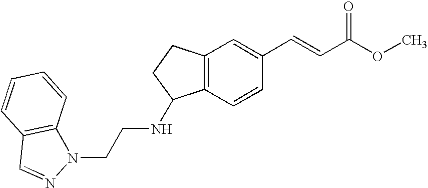 | 2.25 (A) | 362.0 |
| Q56 | 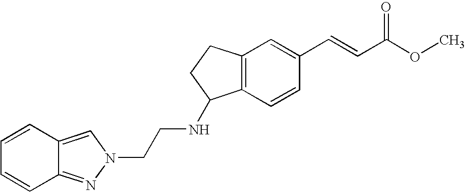 | 2.31 (A) | 361.9 |
| Q57 | 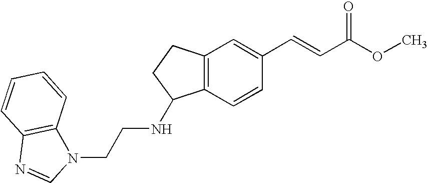 | 1.82 (A) | 362.1 |
| Q58 | 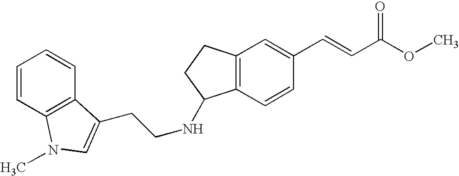 | 1.52 (A) | 375.5 |
| Q59 | 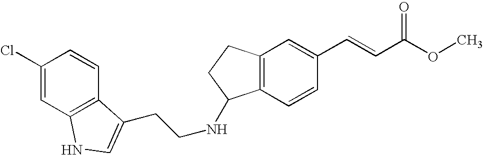 | 2.51 (A) | 395.0 |
| Q60 | 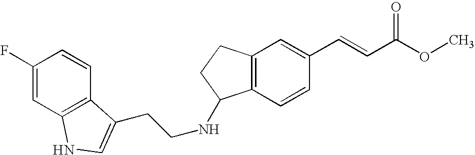 | 2.43 (A) | 379.0 |
| Q61 | 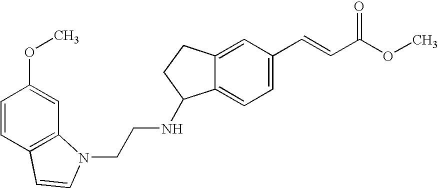 | 2.46 (A) | 391.1 |
| Q62 | 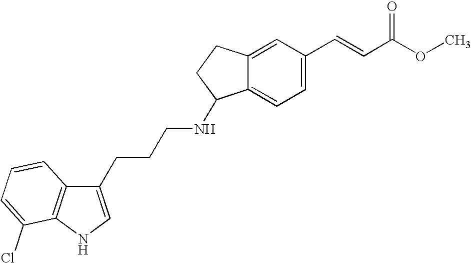 | 2.95 (A) | 409.0 |
| Q63 | 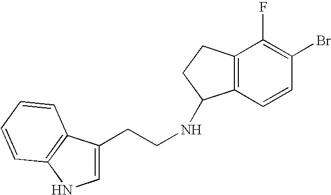 | 2.35 (A) | 373.4 |
| Q64 | 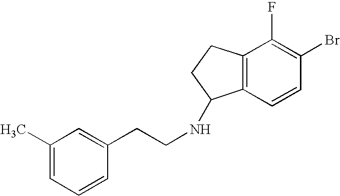 | 2.45 (A) | 347.9 |

### Intermediate R1 and R2

### Chiral Separation of (±) methyl (2E)-3-(5-{(2-hydroxyethyl)[2-(1H-indol-3-yl)ethyl]amino}-5,6,7,8-tetrahydronaphthalen-2-yl)acrylate

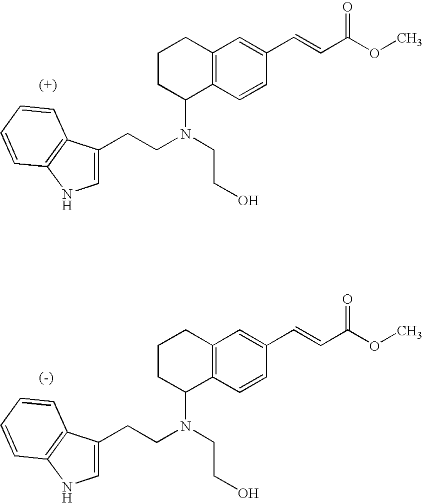

Racemic Intermediate J8, methyl (2E)-3-(5-{(2-hydroxyethyl)[2-(1H-indol-3-yl)ethyl]amino}-5,6,7,8-tetrahydronaphthalen-2-yl)acrylate (0.67 g) was separated with ChiralPAK AD-H 20×250 mm using 35-50% iPrOH in hexane with 0.1% Et₃N (flow rate=15 mL/min, 1600 uL/injection, 90 mg/injection) to obtain the first peak (RT=14.29 min, 295 mg): [α]_{D} (MeOH)=+168.7 (c 1.0). Second peak (RT=17.36 min, 288 mg): [α]_{D} (MeOH)=−172.6 (c 1.0). The overall recovery yield was 86%.

Using procedures similar to the above and what is described for intermediate H1, H2, I1, I2, P1 and P2, the following compounds are prepared in a similar manner.

| Inter- | | Chiral | Separation | RT | [α]_{D} |
|---|---|---|---|---|---|
| mediate | Structure | column | condition | (min) | (solvent) |
| R3 | 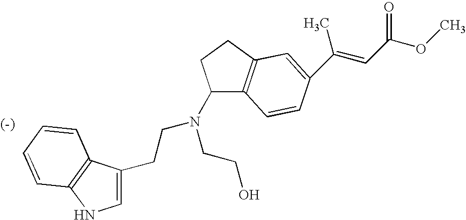 | Chiral PAK AD-H | 15-25% MeOH/EtO H (1/1) in hexanes with 0.1% Et₃N | 16.62 | −66.0 (c 1.0) (MeOH) |
| R4 | 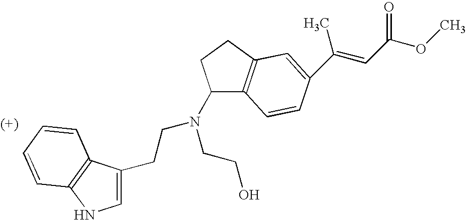 | Chiral PAK AD-H | 15-25% MeOH/EtO H (1/1) in hexanes with 0.1% Et₃N | 20.12 | +66.0 (c 1.0) (MeOH) |
| R5 | 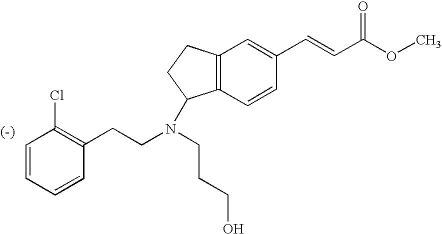 | Chiral PAK AD-H | 20% MeOH/EtO H (1/1) in hexanes with 0.1% Et₃N | 5.74 | * |
| R6 | 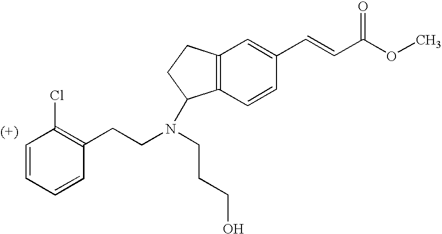 | Chiral PAK AD-H | 20% MeOH/EtO H (1/1) in hexanes with 0.1% Et₃N | 6.93 | * |
| R7 | 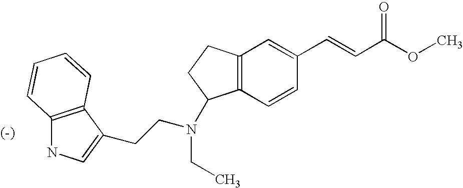 | Chiral PAK AD-H | 10% B (B = 1:1 MeOH:EtOH) in hexane with 0.1% Et₃N | 12.82 | −76.2 (c 1.0) (MeOH) |
| R8 | 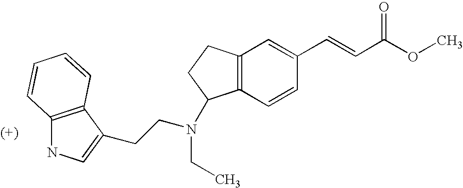 | Chiral PAK AD-H | 10% B (B = 1:1 MeOH:EtOH) in hexane with 0.1% Et₃N | 16.21 | +87.2 (c 1.0) (MeOH) |
| R9 | 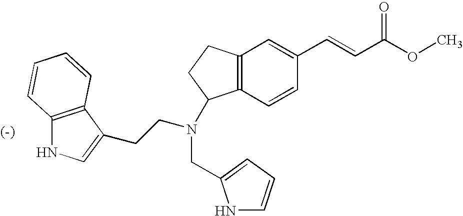 | Chiral PAK AD-H | 70% iPrOH in hexane with 0.1% Et₃N | 12.82 | −76.2 (c 1.0) (MeOH) |
| R10 | 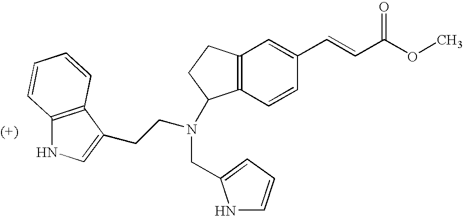 | Chiral PAK AD-H | 70% iPrOH in hexane with 0.1% Et₃N | 12.21 | +25.6 (c 1.0) (MeOH) |
| R11 | 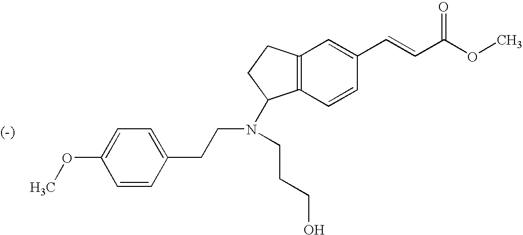 | Chiral PAK AD-H | 25% B (B = 1:1 MeOH:EtOH) in hexane with 0.1% Et₃N | 8.01 | −44.5 (c 1.0) (MeOH) |
| R12 | 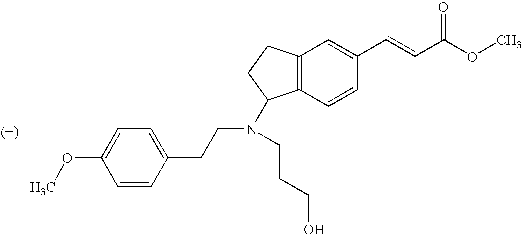 | Chiral PAK AD-H | 25% B (B = 1:1 MeOH:EtOH) in hexane with 0.1% Et₃N | 8.01 | +48.6 (c 1.0) (MeOH) |
| R13 | 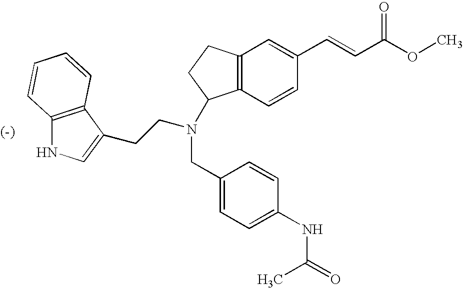 | Chiral PAK AD-H | 15% B (B = 2:1 MeOH:EtOH) in hexane with 0.1% Et₃N | 35.66 | −35.5 (c 1.0) (MeOH) |
| R14 | 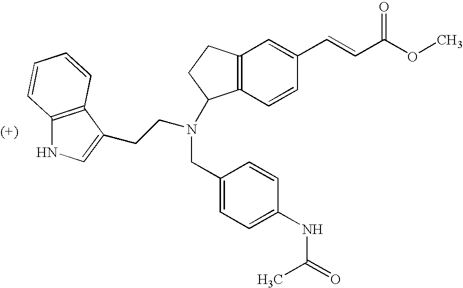 | Chiral PAK AD-H | 15% B (B = 2:1 MeOH:EtOH) in hexane with 0.1% Et₃N | 40.8 | +39.2 (c 1.0) (MeOH) |
| R15 | 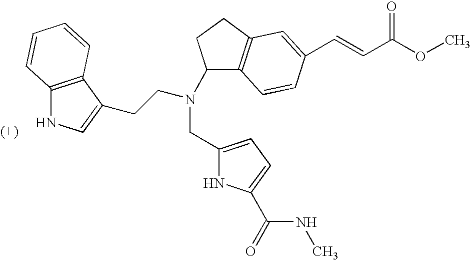 | Chiral PAK AD-H | 35% iPrOH in hexane with 0.1% Et₂NH | 12.20 | +22.0 (c 1.0) (MeOH) |
| R16 | 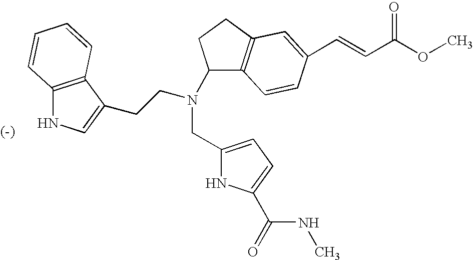 | Chiral PAK AD-H | 35% iPrOH in hexane with 0.1% Et₂NH | 16.01 | −21.9 (c 1.0) (MeOH) |
| R17 | 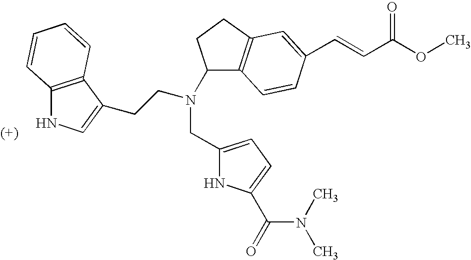 | Chiral PAK AD-H | 40% iPrOH in hexane with 0.1% Et₂NH | 17.48 | +36.4 (c 1.0) (MeOH) |
| R18 | 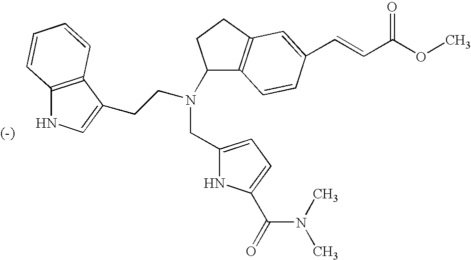 | Chiral PAK AD-H | 40% iPrOH in hexane with 0.1% Et₂NH | 22.87 | −34.8 (c 1.0) (MeOH) |
| R19 | 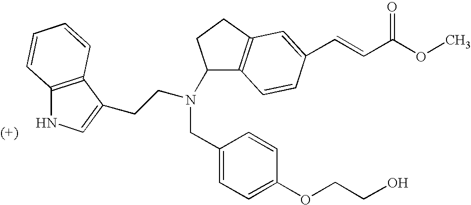 | Chiral PAK AD-H | 80% B (B = 2:1 MeOH:EtOH) in hexane with 0.1% Et₂NH | 25.1 | +20.4 (c 1.0) (MeOH) |
| R20 | 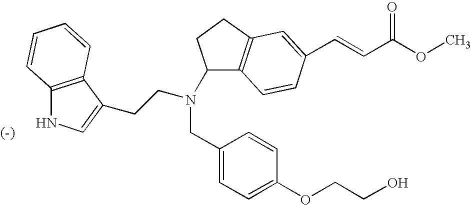 | Chiral PAK AD-H | 80% B (B = 2:1 MeOH:EtOH) in hexane with 0.1% Et₂NH | 39.5 | −20.8 (c 1.0) (MeOH) |
| R21 | 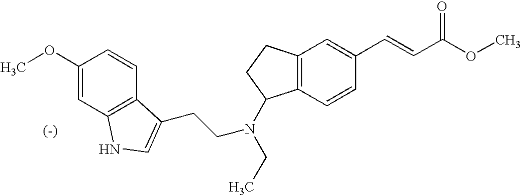 | Chiral Pak AD-H | 20% EtOH in hexanes with 0.1% Et₂NH | 20.30 | −69.9 (c 1.1) (MeOH) |
| R22 | 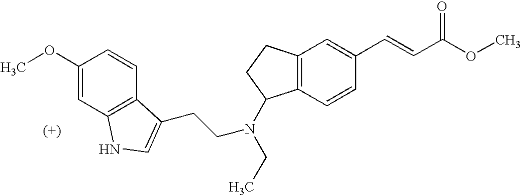 | Chiral Pak AD-H | 20% EtOH in hexanes with 0.1% Et₂NH | 26.60 | +72.2 (c 1.1) (MeOH) |
| R23 | 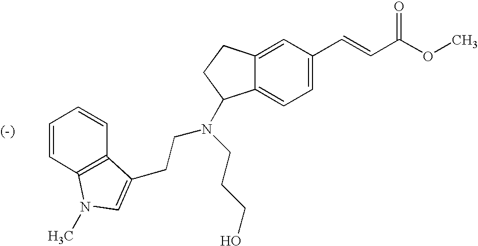 | Chiral Cel OD-H | 25-45% (1:1 MeOH—EtOH) in Hexanes with 0.1% Et₃N | 11.80 | −63.8 (c 1.1) (MeOH) |
| R24 | 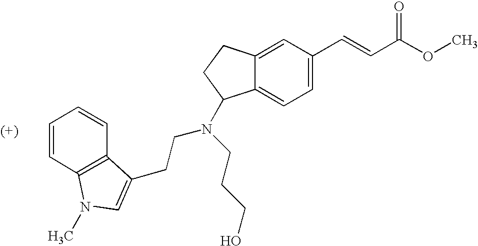 | Chiral Cel OD-H | 25-45% (1:1 MeOH—EtOH) in Hexanes with 0.1% Et₃N | 17.00 | +67.3 (c 1.1) (MeOH) |
| R25 | 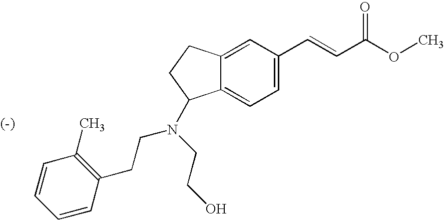 | Chiral Cel OD-H | 15-25% iPrOH in hexanes with 0.1% Et₃N | 23.40 | −69.5 (c 1.3) (MeOH) |
| R26 | 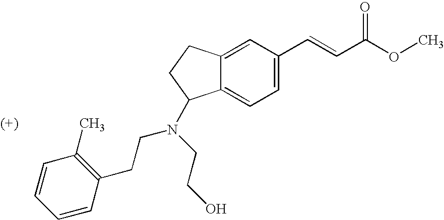 | Chiral Cel OD-H | 15-25% iPrOH in hexanes with 0.1% Et₃N | 13.4 | +75.5 (c 1.3) (MeOH) |
| R27 | 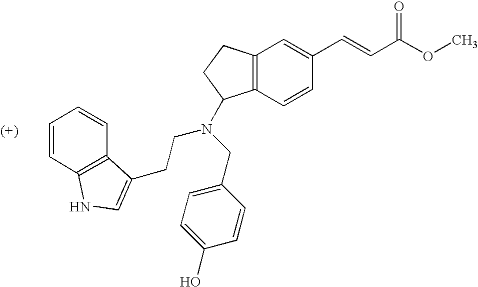 | Chiral Cel OD-H | 25% (1:1 MeOH—EtOH) in hexanes with 0.1% Et₃N | 20.00 | +17.9 (c 0.61) (CH₂Cl₂) |
| R28 | 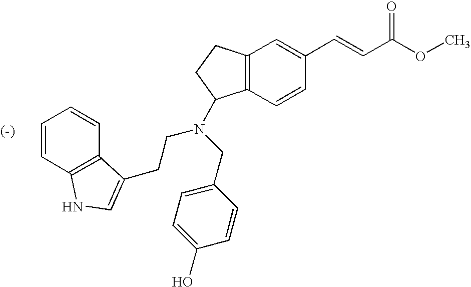 | Chiral Cel OD-H | 25% (1:1 MeOH—EtOH) in hexanes with 0.1% Et₃N | 16.20 | −18.6 (c 0.65) (CH₂Cl₂) |
| R29 | 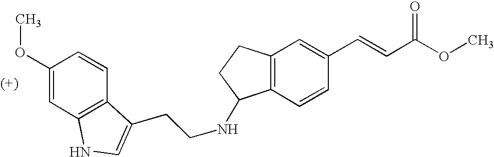 | Chiral Pak AD-H | 35% (1:1 MeOH—EtOH) in hexanes with 0.1% Et₃N | 13.00 | +20.8 (c 1.3) (MeOH) |
| R30 | 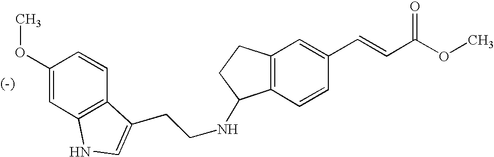 | Chiral Pak AD-H | 35% (1:1 MeOH—EtOH) in hexanes with 0.1% Et₃N | 16.00 | −20.7 (c 1.3) (MeOH) |
| R31 | 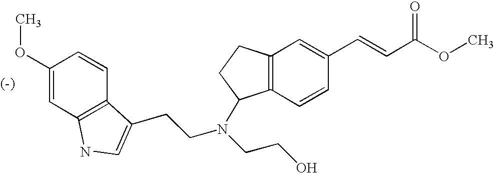 | Chiral Pak AD-H | 70% 1:1 MeOH—EtOH in hexanes with 0.1% Et₃N | 16.60 | −72.8 (c 1.3) (MeOH) |
| R32 | 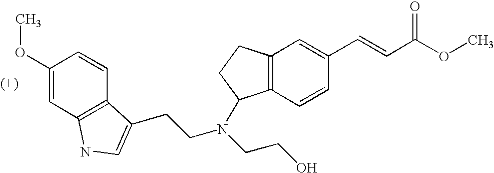 | Chiral Pak AD-H | 70% (1:1 MeOH—EtOH) in hexanes with 0.1% Et₃N | 9.00 | +63.4 (c 1.3) (MeOH) |
| R33 | 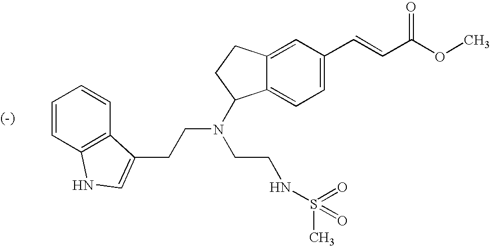 | Chiral Pak AD-H | 40% (2:1 MeOH—EtOH) in hexanes with 0.3% Et₂NH | 15.50 | −56.7 (c 1.1) (MeOH) |
| R34 | 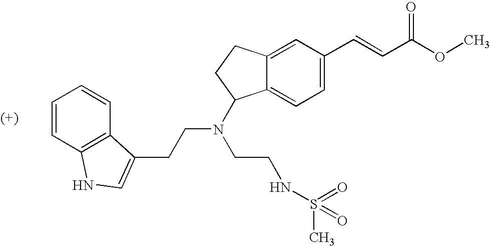 | Chiral Pak AD-H | 40% (2:1 MeOH—EtOH) in hexanes with 0.3% Et₂NH | 22.00 | +57.2 (c 1.3) (MeOH) |
| R35 | 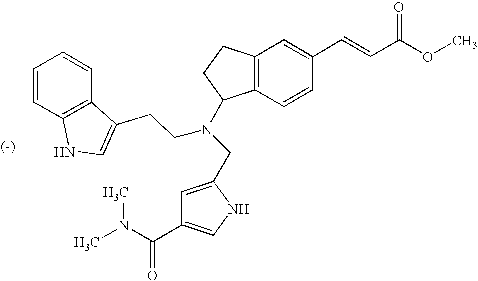 | Chiral Pak AD-H | 45% (2:1 MeOH—EtOH) in hexanes with 0.3% Et₂NH | 14.30 | −7.6 (c 0.9) (MeOH) |
| R36 | 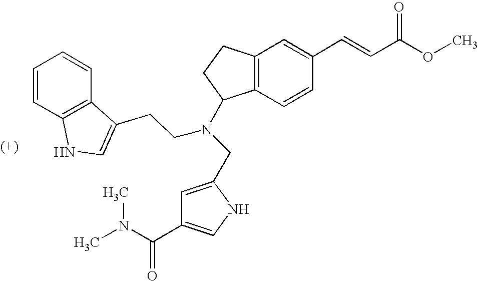 | Chiral Pak AD-H | 45% (2:1 MeOH—EtOH) in hexanes with 0.3% Et₂NH | 19.30 | +8.0 (c 0.9) (MeOH) |
| R37 | 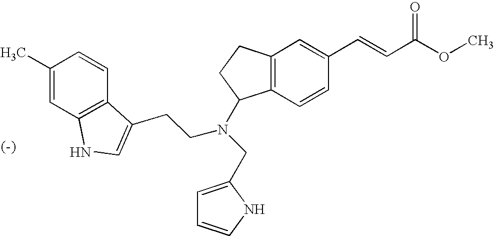 | Chiral PAK AD-H | 70% (4:1 iPrOH:MeOH) in hexanes with 0.1% Et₂NH | 16.20 | −21.0 (c 1.0) (MeOH) |
| R38 | 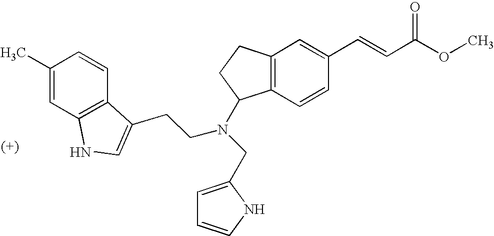 | Chiral PAK AD-H | 70% (4:1 iPrOH:MeOH) in hexanes with 0.1% Et₂NH | 9.08 | +20.8 (c 1.0) (MeOH) |
| R39 | 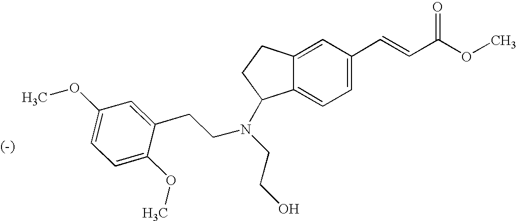 | Chiral PAK AD-H | 25% (MeOH:EtOH, 1:1) in hexane with 0.1% Et₃N | 11.80 | −58.1 (c 1.1) (MeOH) |
| R40 | 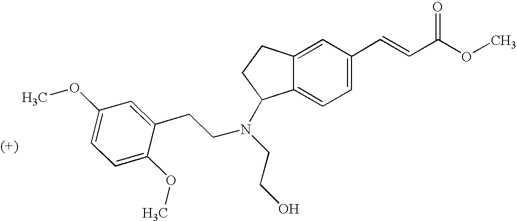 | Chiral PAK AD-H | 25% (MeOH:EtOH, 1:1) in hexane with 0.1% Et₃N | 17.59 | +55.1 (c 1.0) (MeOH) |
| R41 | 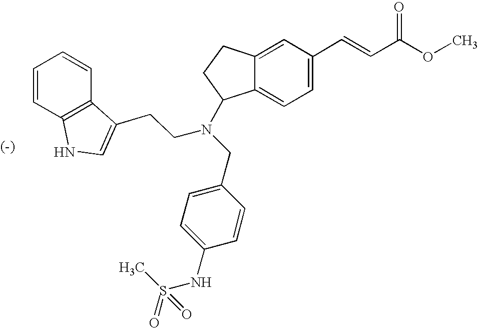 | Chiral PAK AD | 65% iPrOH in hexane with 0.1% Et₃N | 19.92 | −28.4 (c 1.4) (MeOH) |
| R42 | 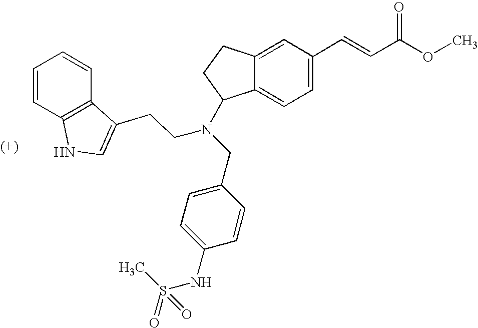 | Chiral PAK AD | 65% iPrOH in hexane with 0.1% Et₃N | 32.82 | +30.5 (c 1.3) (MeOH) |
| R43 | 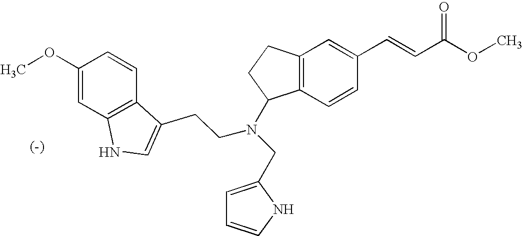 | Chiral PAK AD-H | 70% (4:1 MeOH:EtOH) in hexanes with 0.1% Et₃N | 34.40 | −21.0 (c 1.0) (MeOH) |
| R44 | 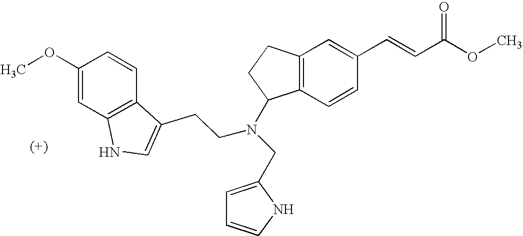 | Chiral PAK AD-H | 70% (4:1 MeOH:EtOH) in hexanes with 0.1% Et₂NH | 21.06 | +20.8 (c 1.0) (MeOH) |
| R45 | 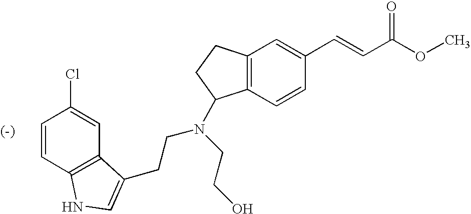 | | | | −79.3 (c 0.7) (MeOH) |
| R46 | 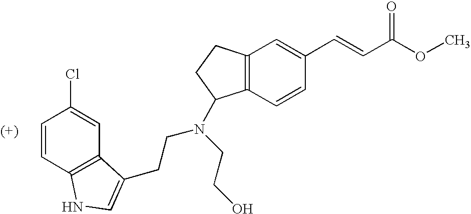 | | | | +83.0 (c 1.0) (MeOH) |
| R47 | 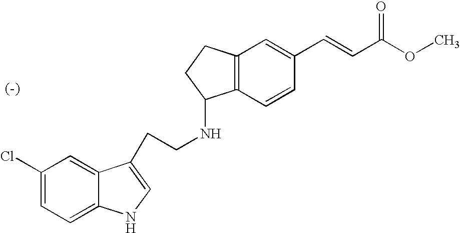 | | | | −124.0 (c 1.0) (MeOH) |
| R48 | 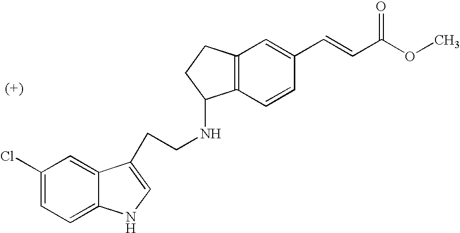 | | | | +164.0 (c 1.0) (MeOH) |
| R49 | 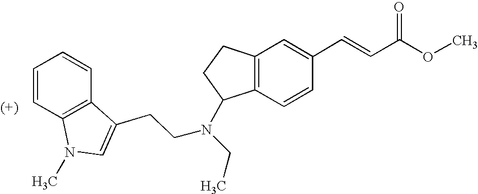 | Chiral Pak AD-H | 5% (4:1 iPrOH/MeOH) in hexanes with 0.1% Et₂NH | 21.30 | +75.5 (c 1.4) (MeOH) |
| R50 | 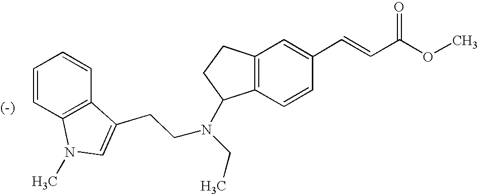 | Chiral Pak AD-H | 5% (4:1 iPrOH/MeOH) in hexanes with 0.1% Et₂NH | 16.70 | −74.4 (c 1.4) (MeOH) |
| R51 | 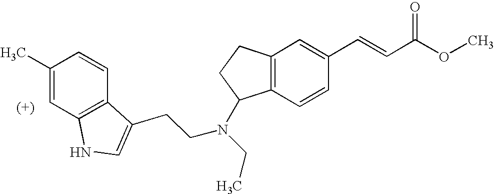 | Chiral PAK AD-H | 15% EtOH in hexanes with 0.1% Et₃N | 20.40 | +66.2 (c 1.0) (MeOH) |
| R52 | 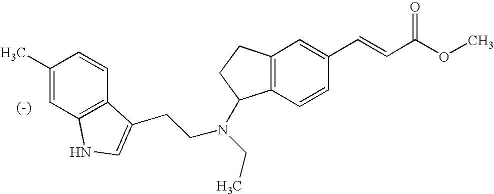 | Chiral PAK AD-H | 15% EtOH in hexanes with 0.1% Et₃N | 16.70 | −70.8 (c 0.8) (MeOH) |
| R53 | 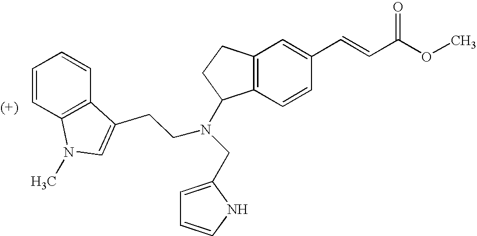 | | | | |
| R54 | 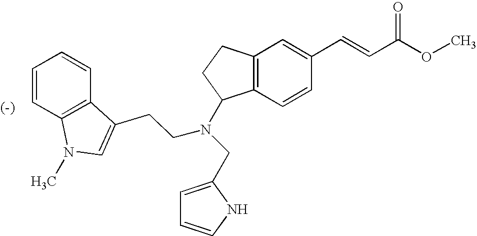 | | | | |
| R55 | 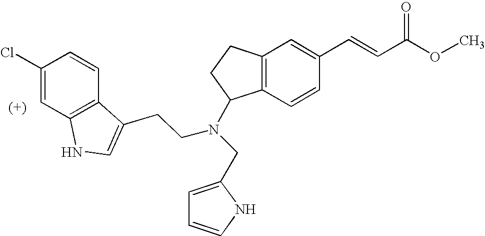 | Chiral PAK AD-H | 50% (4:1 iPrOH:MeOH) in hexanes with 0.1% Et₂NH | 17.50 | +27.0 (c 1.0) (MeOH) |
| R56 | 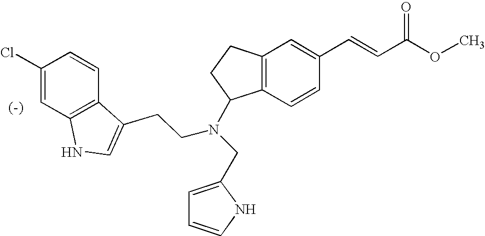 | Chiral PAK AD-H | 50% (4:1 iPrOH:MeOH) in hexanes with 0.1% Et₂NH | 33.70 | −20.6 (c 1.0) (MeOH) |
| R57 | 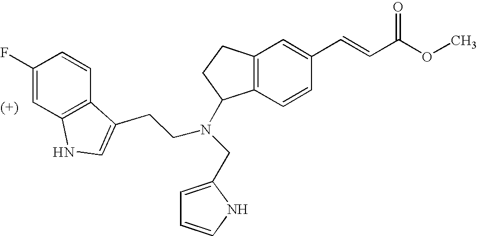 | Chiral Pak AD-H | 50% (4:1 iPrOH/MeOH) in hexanes with 0.1% Et₂NH | 17.40 | +21.8 (c 1.1) (MeOH) |
| R58 | 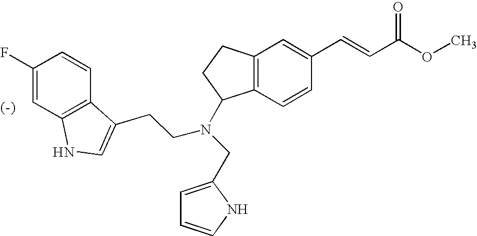 | Chiral Pak AD-H | 50% (4:1 iPrOH/MeOH) in hexanes with 0.1% Et₂NH | 27.70 | −23.5 (c 1.1) (MeOH) |
| R59 | 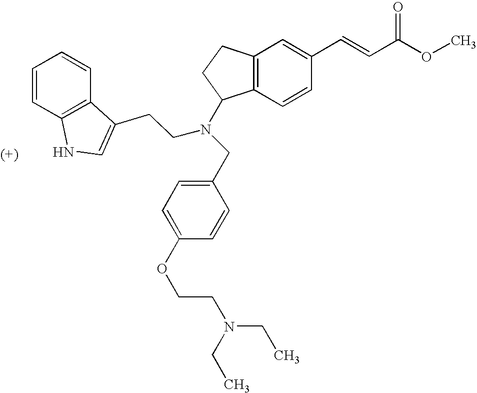 | Chiral PAK AD-H | 60% B (B = 9:1 EtOH:MeOH) in hexanes with 0.1% Et₃N | 18.70 | +17.4 (c 1.0) (MeOH) |
| R60 | 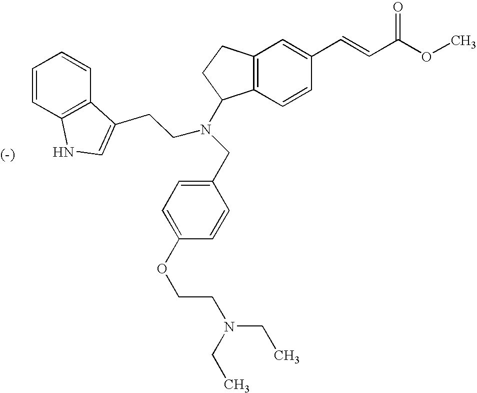 | Chiral PAK AD-H | 60% B (B = 9:1 EtOH:MeOH) in hexanes with 0.1% Et₃N | 14.20 | −13.6 (c 1.0) (MeOH) |
| R61 | 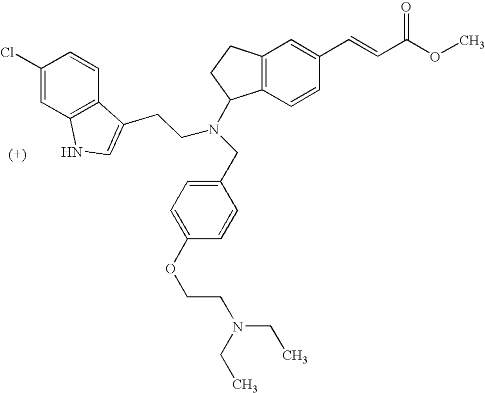 | Chiral PaK AD-H | 40% (9:1 EtOH/MeOH) in hexanes with Et₃N | 23.80 | +15.4 (c 1.3) (MeOH) |
| R62 | 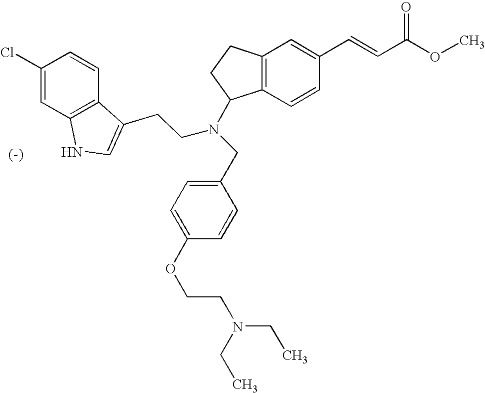 | Chiral PaK AD-H | 40% (9:1 EtOH/MeOH) in hexanes with Et₃N | 16.20 | −19.5 (c 1.3) (MeOH) |
| R63 | 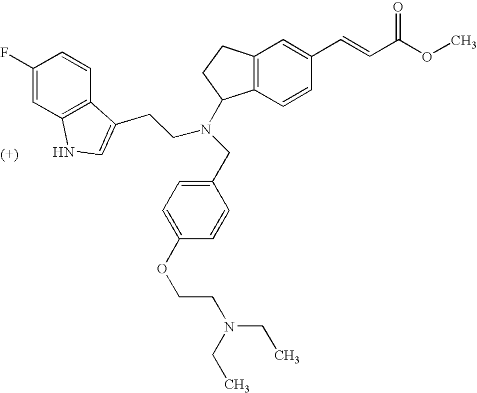 | Chiral Pak AD-H | 35% (9:1 EtOH—MeOH) in hexanes with 0.1% Et₂NH | 14.20 | +27.3 (c 1.0) (MeOH) |
| R64 | 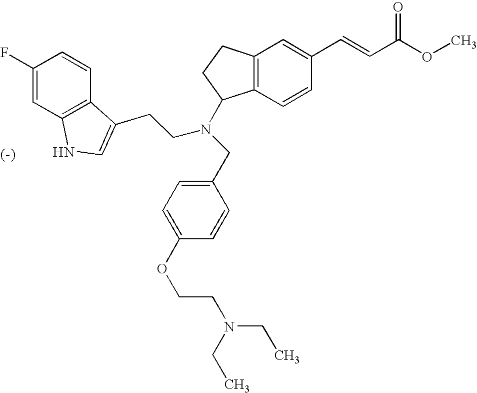 | Chiral Pak AD-H | 35% (9:1 EtOH—MeOH) in hexanes with 0.1% Et₂NH | 7.10 | −26.8 (c 1.0) (MeOH) |
| R65 | 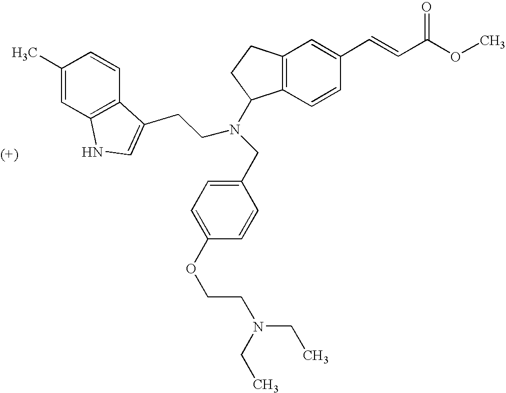 | Chiral Pak AD-H | 100% (2:1 MeOH—iPrOH) with 0.1% Et₃N | 32.00 | +16.5 (c 1.0) (MeOH) |
| R66 | 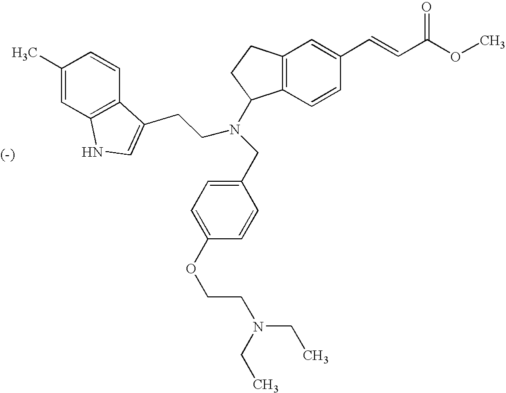 | Chiral Pak AD-H | 100% (2:1 MeOH—iPrOH) with 0.1% Et₃N | 14.50 | −16.1 (c 1.0) (MeOH) |
| R67 | 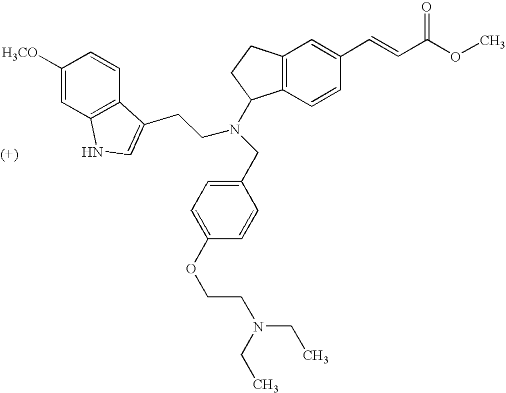 | Chiral Pak AD-H | 80% (1:1 MeOH—EtOH) in hexanes with 0.1% Et₂NH | 21.81 | +21.3 (c 1.0) (MeOH) |
| R68 | 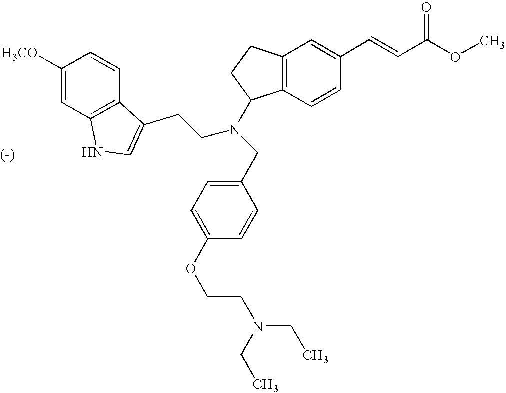 | Chiral Pak AD-H | 80% (1:1 MeOH—EtOH) in hexanes with 0.1% Et₂NH | 13.59 | −21.7 (c 0.9) (MeOH) |
| R69 | 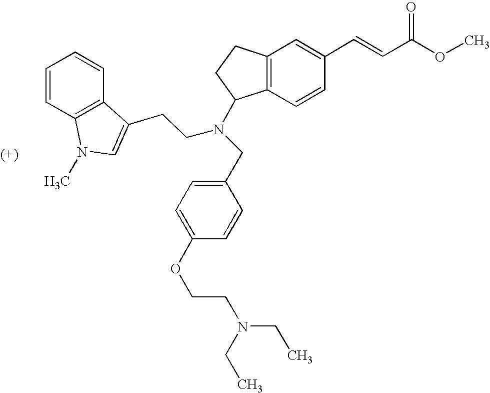 | Chiral Pak AD-H | 15% iPrOH in hexanes with 0.1% Et₃N | 13.17 | +24.6 (c 0.8) (MeOH) |
| R70 | 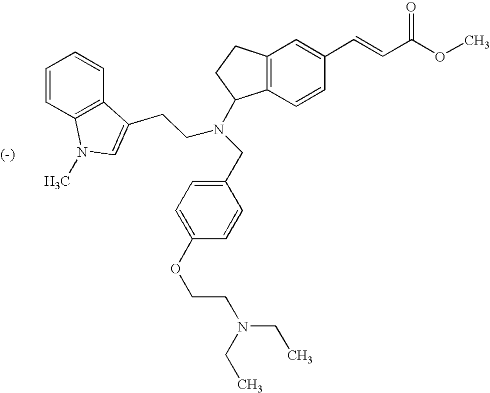 | Chiral Pak AD-H | 15% iPrOH in hexanes with 0.1% Et₃N | 19.37 | −25.0 (c 0.6) (MeOH) |
| R71 | 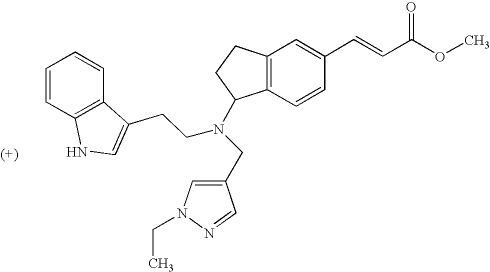 | Chiral Pak AD-H | 50% (4:1 iPrOH—MeOH) in hexanes with 0.1% Et₂NH | 8.79 | +19.0 (c 0.1) (MeOH) |
| R72 | 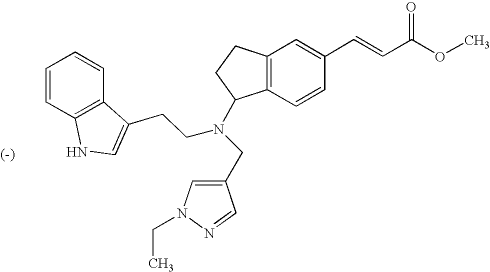 | Chiral Pak AD-H | 50% (4:1 iPrOH—MeOH) in hexanes with 0.1% Et₂NH | 16.63 | −15.0 (c 0.1) (MeOH) |
| R73 | 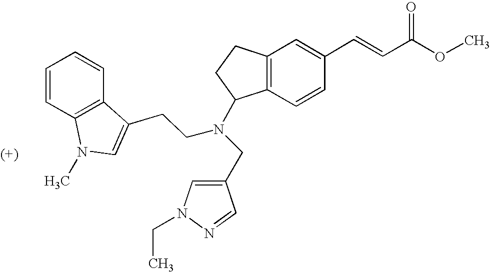 | Chiral Pak AD-H | 20% (1:1 MeOH—EtOH) in hexanes with 0.1% Et₃N | 10.00 | +13.0 (c 0.1) (MeOH) |
| R74 | 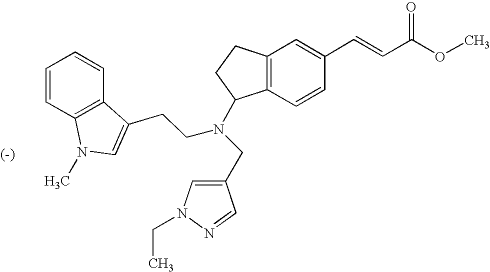 | Chiral Pak AD-H | 20% (1:1 MeOH—EtOH) in hexanes with 0.1% Et₃N | 14.50 | −10.0 (c 0.1) (MeOH) |
| R75 | 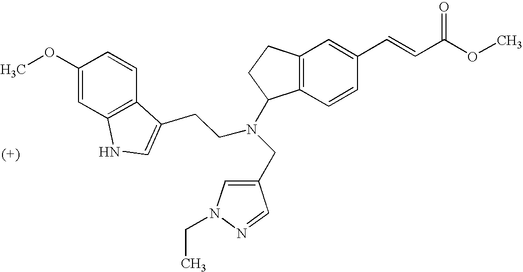 | Chiral Pak AD | 70% (4:1 MeOH—iPrOH) in hexanes with 0.1% Et₂NH | 14.00 | +12.0 (c 0.1) (MeOH) |
| R76 | 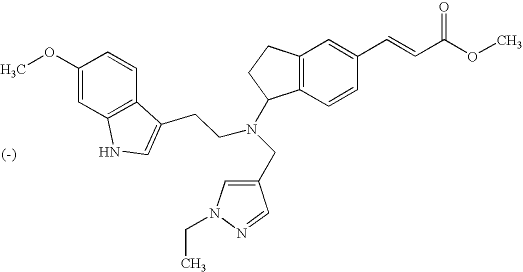 | Chiral Pak AD | 70% (4:1 MeOH—iPrOH) in hexanes with 0.1% Et₂NH | 25.40 | −14.0 (c 0.1) (MeOH) |
| R77 | 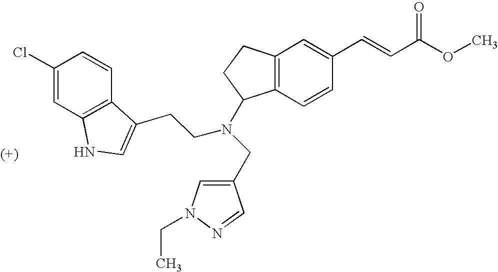 | Chiral PAK AD-H | 40% (1:1 MeOH—EtOH)/ Hexanes 0.1% Et₃N | 11.10 | +5.5 (c 1.0) (MeOH) |
| R78 | 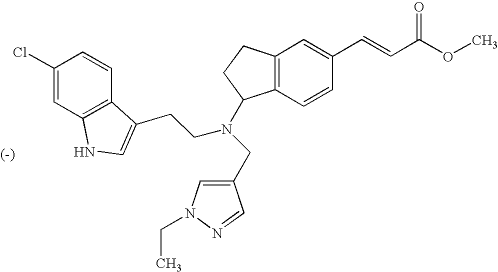 | Chiral PAK AD-H | 40% (1:1 MeOH—EtOH)/ Hexanes 0.1% Et₃N | 22.80 | −4.5 (c 1.0) (MeOH) |
| R79 | 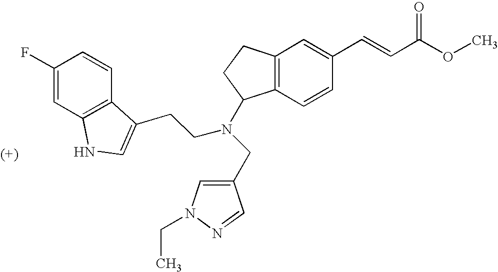 | Chiral PAK AD-H | 40% (1:1 MeOH—EtOH) in hexanes with 0.1% Et₃N | 8.10 | +5.2 (c 1.0) (MeOH) |
| R80 | 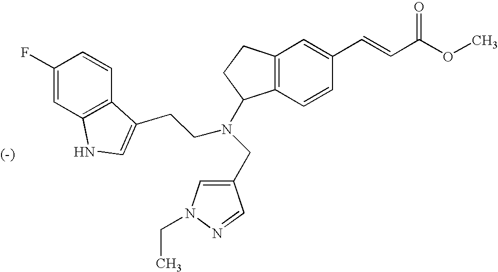 | Chiral PAK AD-H | 40% (1:1 MeOH—EtOH) in hexanes with 0.1% Et₃N | 16.50 | −8.7 (c 1.0) (MeOH) |
| R81 | 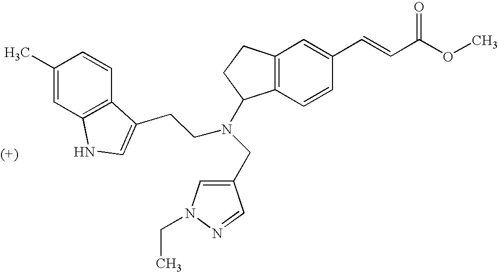 | Chiral PAK AD | 50% (4:1 iPrOH—MeOH) in hexanes with 0.1% Et₃N | 10.30 | +15.0 (c 0.1) (MeOH) |
| R82 | 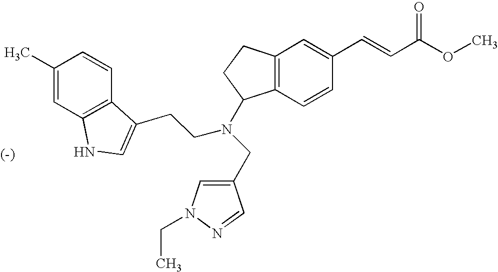 | Chiral PAK AD | 50% (4:1 iPrOH—MeOH) in hexanes with 0.1% Et₃N | 20.80 | −13.0 (c 0.1) (MeOH) |
| R83 | 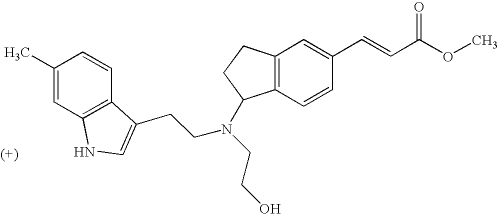 | Chiral PAK AD-H | 35% B (B = 4:1 iPrOH:MeOH) in hexanes with 0.1% Et₂NH | 17.50 | +58.5 (c 1.0) (MeOH) |
| R84 | 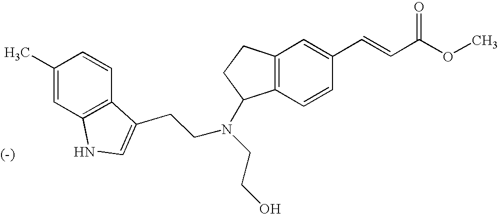 | Chiral PAK AD-H | 35% B (B = 4:1 iPrOH:MeOH) in hexanes with 0.1% Et₂NH | 33.70 | −64.3 (c 1.0) (MeOH) |
| R85 | 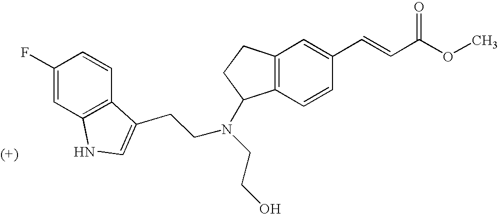 | Chiral Pak AD-H | 40% (4:1 iPrOH/MeOH) in hexanes with Et₂NH | 19.10 | +70.8 (c 1.1) (MeOH) |
| R86 | 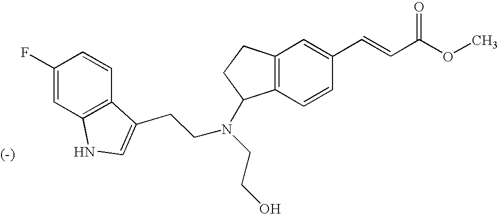 | Chiral Pak AD-H | 40% (4:1 iPrOH/MeOH) in hexanes with Et₂NH | 25.60 | −67.6 (c 1.1) (MeOH) |
| R87 | 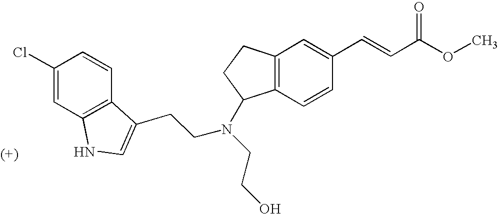 | Chiral Pak AD | 30% (4:1 iPrOH—MeOH) in hexanes | 12.38 | +71.6 (c 1.1) (MeOH) |
| R88 | 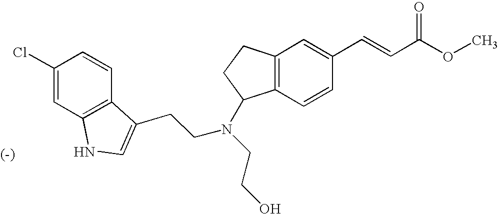 | Chiral Pak AD | 30% (4:1 iPrOH—MeOH) in hexanes | 18.93 | −64.4 (c 1.0) (MeOH) |
| R89 | 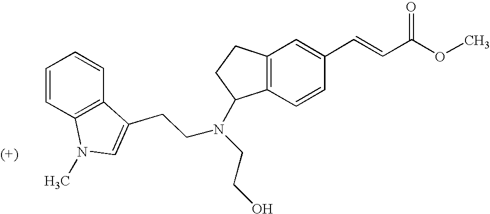 | Chiral Cel OD-H | 20% (4:1 iPrOH/MeOH) in hexanes with Et₂NH | 28.10 | +67.2 (c 1.4) (MeOH) |
| R90 | 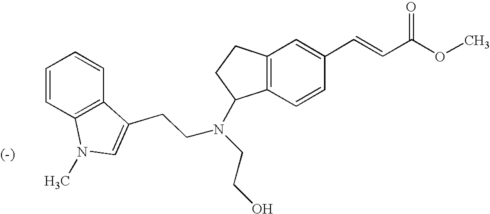 | Chiral Cel OD-H | 20% (4:1 iPrOH/MeOH) in hexanes with Et₂NH | 33.10 | −61.2 (c 1.5) (MeOH) |
| R91 | 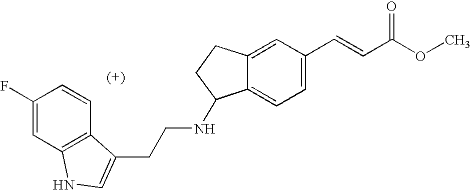 | Chiral Pak AD-H | 20% (3:1 MeOH—EtOH) in hexanes with 0.1% Et₃N | 15.75 | +18.8 (c 0.6) (MeOH) |
| R92 | 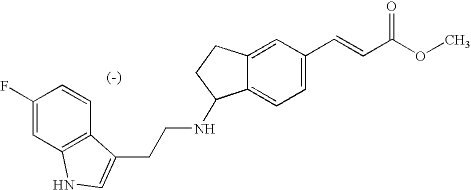 | Chiral Pak AD-H | 20% (3:1 MeOH—EtOH) in hexanes with 0.1% Et₃N | 20.75 | −18.9 (c 0.6) (MeOH) |
| R93 | 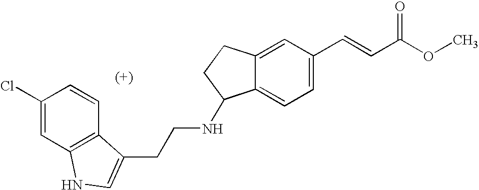 | Chiral PAK AD-H | 20% B (B = 3:1 MeOH:EtOH) in hexanes with 0.1% Et₃N | 18.20 | +20.5 (c 1.0) (MeOH) |
| R94 | 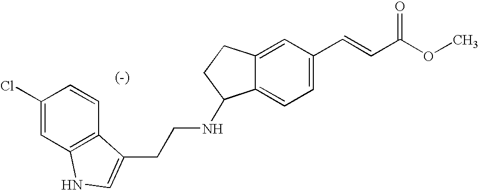 | Chiral PAK AD-H | 20% B (B = 3:1 MeOH:EtOH) in hexanes with 0.1% Et₃N | 23.40 | −23.0 (c 1.0) (MeOH) |
| R95 | 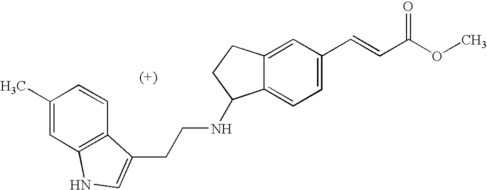 | Chiral Pak AD-H | 15% (3:1 MeOH—EtOH) in hexanes with 0.1% Et₃N | 27.30 | +21.1 (c 0.8) (MeOH) |
| R96 | 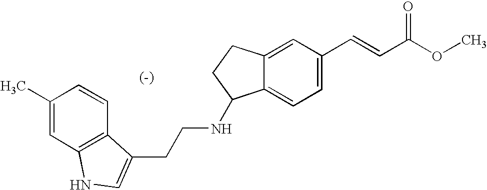 | Chiral Pak AD-H | 15% (3:1 MeOH—EtOH) in hexanes with 0.1% Et₃N | 33.30 | −19.8 (c 0.9) (MeOH) |
| R97 | 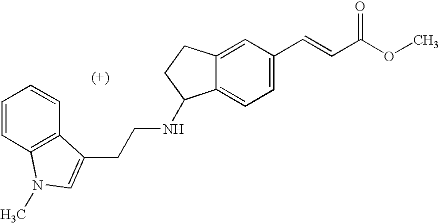 | Chiral Pak AD-H | 15% (1:1 MeOH/EtOH) in hexanes with 0.1% Et₂NH | 10.00 | +18.3 (c 1.3) (MeOH) |
| R98 | 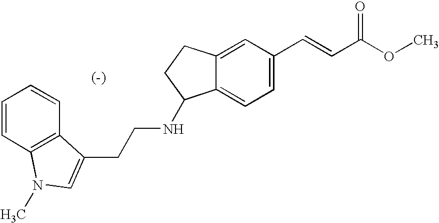 | Chiral Pak AD-H | 15% (1:1 MeOH/EtOH) in hexanes with 0.1% Et₂NH | 14.00 | −14.9 (c 1.2) (MeOH) |
| R99 | 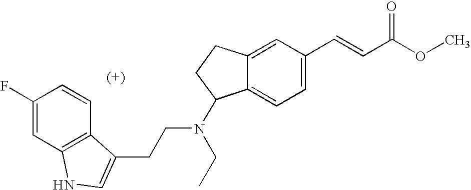 | Chiral Pak AD-H | 15% EtOH in Hexanes with 0.1% Et₂NH | 14.80 | +52.9 (c 1.1) (MeOH) |
| R100 | 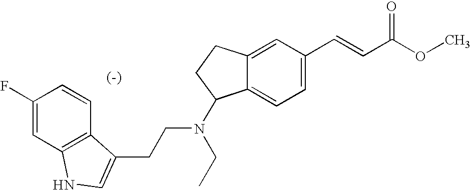 | Chiral Pak AD-H | 15% EtOH in Hexanes with 0.1% Et₂NH | 11.30 | −68.4 (c 1.2) (MeOH) |
| R101 | 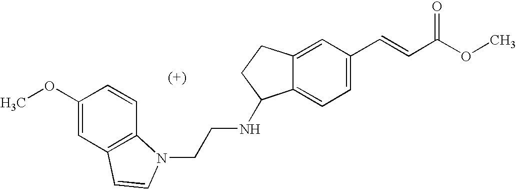 | Chiral Pak AD-H | 25% (3:1 MeOH—EtOH) in Hexane with 0.1% Et₃N | 19.95 | +22.2 (c 1.0) (MeOH) |
| R102 | 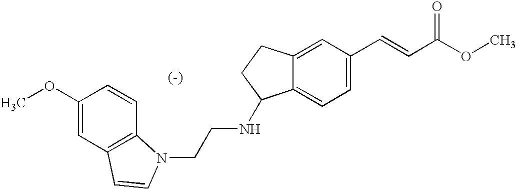 | Chiral Pak AD-H | 25% (3:1 MeOH—EtOH) in Hexane with 0.1% Et₃N | 26.09 | −23.9 (c 1.0) (MeOH) |

The optical rotations were not measured at the methyl ester stage. Assignments were made based on the optical rotation of the final hydroxamic acids.

### Intermediate S

### Methyl 3-(1-{(2-hydroxyethyl)[2-(1H-indol-3-yl)ethyl]amino}-2.3-dihydro-1H-inden-5-yl)propanoate

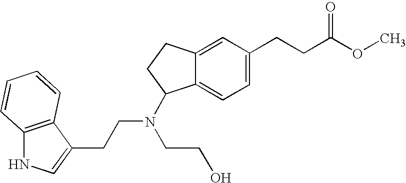

To a stirred solution mixture of intermediate J [methyl (2E)-3-(1-{(2-hydroxyethyl)[2-(1H-indol-3-yl)ethyl]amino}-2,3-dihydro-1H-inden-5-yl)acrylate] (300 mg, 0.74 mmol) and CuCl (55 mg, 0.56 mmol) in MeOH (5 mL) and THF (2.5 mL) at 0° C. was added NaBH₄ (280 mg, 7.42 mmol). The black mixture was allowed to stir at 0 ° C. for 1 h. The resulting black precipitate was removed by filtration, and the filtrate was acidified with 1 N HCl solution. White precipitate formed and saturated NaHCO₃ was added to dissolve it. The mixture was extracted with EtOAc three times. The combined organic layer was washed with water, dried over Na₂SO₄, filtered and concentrated to obtain a colorless oil. The crude product was resubmitted to the same condition as described above two more times. After the third time, the reaction was worked up as described above and the crude residue was purified with preparative TLC [10% MeOH (with 2M NH₃) in CH₂Cl₂ to obtain methyl 3-(1-{(2-hydroxyethyl)[2-(1H-indol-3-yl)ethyl]amino}2,3-dihydro-1H-inden-5-yl)propanoate as a colorless oil (65 mg, 21%): LC/MS [M+H] 407.1, RT 2.35 min (method A). ¹H-NMR (CD₃OD) δ 7.33 (m, 2H), 7.21 (d, J=5.7 Hz, 1H), 7.05 (m, 5H), 4.62 (t, 1H), 3.62 (s, 3H), 3.58 (m, 2H), 2.86 (m, 12H), 2.22 (m, 1H), 2.02 (m, 1H).

### Intermediate T

### (Methyl (2E)-3-(1-{(2-aminoethyl)[2-(1H-indol-3-yl)ethyl]amino}-2,3-dihydro-1H-inden-5-yl)acrylate

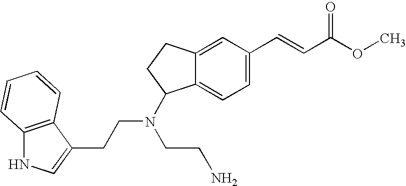

Intermediate F2 [methyl (2E)-3-(1-{{2-[(tert-butoxycarbonyl)amino]ethyl}[2-(1H-indol-3-yl)ethyl]amino}-2,3-dihydro-1H-inden-5yl)acrylate] (6.80 g, 13.5 mmol) was dissolved in anhydrous MeOH (20 mL) and cooled to 0° C. HCl (4N in dioxane, 20 mL) was added to the solution and the resulting mixture was warmed to rt. The reaction was then warmed to 40° C. for 1 h at which time the solvent was removed under vacuum and the crude solid was triturated with Et₂O and collected by filtration. The solid was dissolved in a biphasic mixture of EtOAC/saturated NaHCO₃ solution. The organic layer was collected, dried over anhydrous Na₂SO₄, filtered, followed by removal of solvent to give methyl (2E)-3-(1-{(2-aminoethyl)[2-(1H-indol-3-yl)ethyl]amino}-2,3-dihydro-1H-inden-5-yl)acrylate (3.70 g, 68% yield) as a yellow solid: LC/MS [M+H] 404.1, RT 0.97 min (method A). ¹H-NMR (CD₂Cl₂) δ 8.20 (br s, 1H), 7.67 (d, 1 H), 7.45-7.29 (m, 5H), 7.13 (m, 1H), 7.03-6.99 (m, 2H), 6.43 (d, 1H), 4.64 (t, 1H), 3.79 (s, 3H), 3.05-2.50, (m, 10H), 2.24 (m, 1H), 2.02 (m, 1H).

### Intermediate U

### Methyl (2E)-3-(1-{[2-(2-chlorophenoxy)ethyl][2-(1H-indol-3-yl)ethyl]amino}-2,3-dihydro-1H-inden-5-yl)acrylate

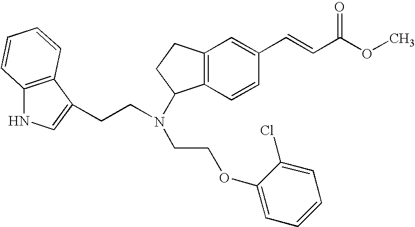

To a solution of intermediate J [methyl (2E)-3-(1{(2-hydroxyethyl)[2-(1 H-indol-3-yl)ethyl]amino}-2,3-dihydro-1H-inden-5-yl)acrylate] (150 mg, 0.37 mmol) in THF (3 mL) was added 2-chlorophenol (52 mg, 0.41 mmol), triphenylphosphine (194 mg, 0.74 mmol) and ADDP (187 mg, 0.74 mmol). The mixture was left to stir under nitrogen overnight. HPLC analytical showed complete conversion of starting material to product. Hexane (9 mL) was added to the mixture. The solid was filtered off and the filtrated was concentrated in vacuum. The crude residue was purified with 25 M Biotage eluting with 25% EtOAc in hexane to obtain methyl (2E)-3-(1-{[2-(2-chlorophenoxy)ethyl][2-(1H-indol-3-yl)ethyl]amino}-2,3-dihydro-1H-inden-5-yl)acrylate as a pale yellow oil (160 mg, 84%): LC/MS [M+H] 515.1, RT 2.66 min (method A). ¹H-NMR (DMSO-d6) δ 10.70 (s, 1H), 7.635 (d, J=12 Hz, 1H), 7.54 (s, 1H), 7.47 (d, J=6.3 Hz, 1H), 7.39 (dd, J=6 Hz and 1.2 Hz, 1H), 7.31 (m, 4H), 7.10 (d, J=1.8 Hz, 1H), 7.01 (m, 2H), 6.92 (m, 2H), 6.575 (d, J=12 Hz, 1H), 4.66 (t, 1H), 4.08 (t, 2H), 3.69 (s, 3H), 2.89 (m, 8H), 2.26 (m, 1H), 1.96 (m, 1H).

The following compounds are synthesized in a similar manner as described above.

| Inter- | | HPLC RT | |
|---|---|---|---|
| mediate | Structure | (min) (method) | M + H |
| U1 | 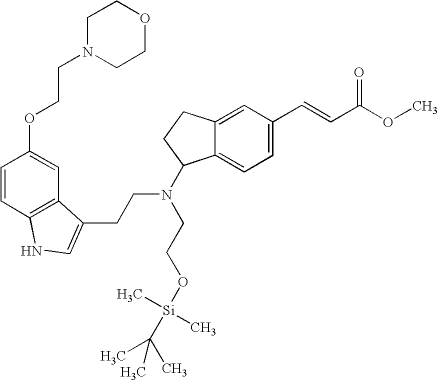 | TLC: R_{f}=0.77 (EtOAc: Hex, 1:1) | |
| U2 | 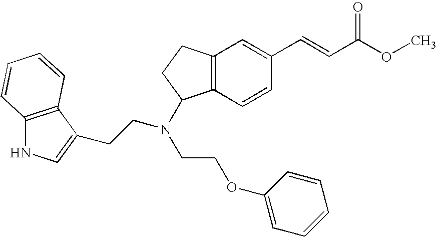 | 2.69 (A) | 481.1 |
| U3 | 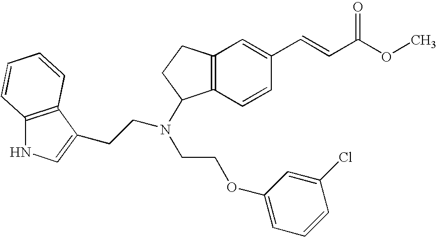 | 2.71 (A) | 515.1 |
| U4 | 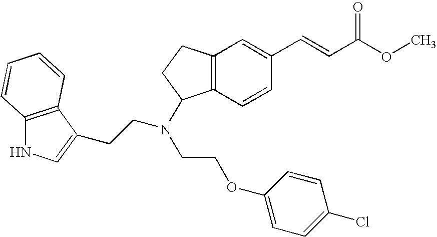 | 2.69 (A) | 515.2 |
| U5 | 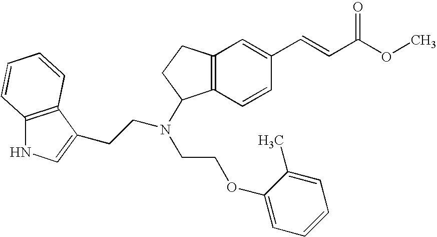 | 2.70 (A) | 495.2 |
| U6 | 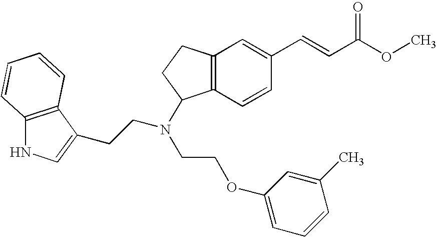 | 2.68 (A) | 495.2 |
| U7 | 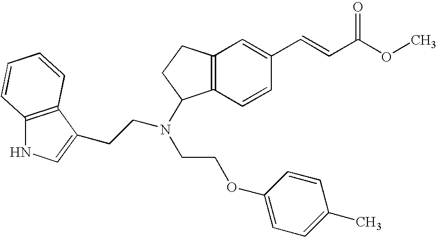 | 2.69 (A) | 495.1 |

### Intermediate V

### methyl (2E)-3-(1-methyl[2-(1-methyl-1H-indol-3-yl)ethyl]amino}-2,3-dihydro-1H-inden-5-yl)acrylate

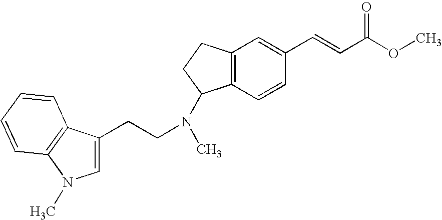

To a cold solution of intermediate E [methyl (2E)-3-(1-{[2-(1H-indol-3-yl)ethyl]amino}-2,3-dihydro-1H-inden-5-yl)acrylate] (1.15 g, 3.19 mmol) in THF (30 mL) was added NaH (0.36 g, 8.93 mmol). The reaction mixture was stirred for 5 min and then Mel (1.36 g, 9.57 mmol) was added. The reaction mixture was stirred at 0° C. for 1 h and then at rt for 2 h. Saturated NH₄Cl and ice water were added and the mixture was extracted with EtOAc. The organic layer was washed with brine, dried over Na₂SO₄, filtered and concentrated down. Chromatography using a Biotage cartridge (25S) with the EtOAct Hexane (40%) afforded methyl (2E)-3-(1-{methyl[2-(1-methyl-1H-indol-3-yl)ethyl]amino}-2,3-dihydro-1H-inden-5-yl)acrylate (0.59 g, 47%). LC/MS [M+H] 389.0, RT 2.51 min (method A). ¹H-NMR (DMSO-d6) δ 7.61 (d, 1 H), 7.54 (s, 1 H), 7.48 (s, 1H), 7.38 (s, 1H), 7.33 (d, 1H), 7.23 (s, 1H), 7.07 (m, 2H), 6.93 (m, 1H), 6.56 (d, 1H), 4.43 (t, 1H), 3.70 (s, 3H), 3.69 (s, 3H), 2.71˜2.92 (m, 4H), 2.60 (t, 2H), 2.25 (s, 3H), 2.05 (m, 1H), 1.95 (m, 1H).

The following compounds are prepared in a similar manner as described above.

| Inter- | | HPLC RT | |
|---|---|---|---|
| mediate | Structure | (min) (method) | M + H |
| V1 | 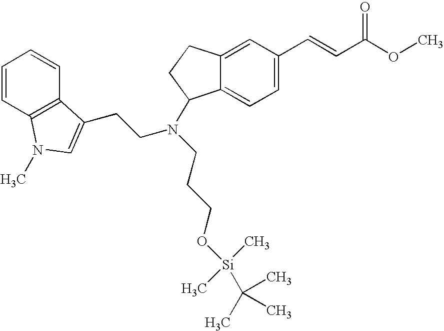 | 3.25 (A) | 547.5 |

### Intermediate W

### Methyl (2E)-3-{1-[(2-hydroxyethyl)(2-phenoxyethyl)amino]-2,3-dihydro-1H-inden-5-yl}acrylate

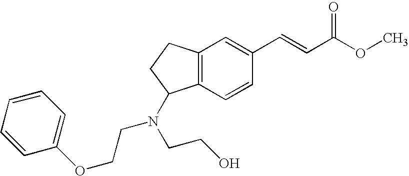

Intermediate F168 [methyl (2E)-3-{1-[(2-{[tert-butyl(dimethyl)silyl]oxy}ethyl)(2-hydroxyethyl)amino]-2,3-dihydro-1H-inden-5-yl}acrylate] (120 mg, 0.29 mmol), phenol (32 mg, 0.34 mmol), triphenylphosphine (150 mg, 0.57 mmol), and ADDP (144 mg, 0.57 mmol) were dissolved in CH₂Cl₂ and stirred for 18 h. Hexanes were added to the reaction mixture to precipitate triphenylphosphine oxide. The crude reaction mixture was then filtered and the filtrate was adsorbed onto silica supported tosic acid (Si-Tosic acid) Silicycle inc. (2 g) in a Baker SPE cartridge. The Si-Tosic acid was eluted with CH₂Cl₂ (50 mL), and with MeOH (50 mL) and the eluent was discarded. After 12 h, the Si-Tosic acid was eluted with 2 N NH₃ in MeOH (30 mL) and the eluent was collected and the solvent removed under vacuum. The crude material was dissolved in EtOAc and was washed with saturated NaHCO₃ solution, and brine. The organic layer was collected, dried over anhydrous Na₂SO₄, filtered, followed by removal of solvent under vacuum. The product was purified further by 25 M Biotage eluting with 40% EtOAc/hexanes with 1% 2N NH₃ in MeOH added to obtain methyl (2E)-3-{1-[(2-hydroxyethyl)(2-phenoxyethyl)amino]-2,3-dihydro-1H-inden-5-yl}acrylate as a light yellow oil (72 mg, 66% yield): LC/MS [M+H] 382.0, RT 2.19 min (method A). ¹H-NMR (CD₂Cl₂) δ 7.67 (d, 1H), 7.42 (br s, 1H), 7.38 (br s, 2H), 7.28 (m 2H), 6.95 (m, 1H), 6.89 (m, 2H), 6.43 (m, 1H), 4.63 (t, 1H), 4.10-3.99 (m, 2H), 3.79 (s, 3H), 3.66 (m, 1H), 3.50 (m, 1H), 3.03-2.83 (m, 4H), 2.73 (m, 1H), 2.32 (m, 1H), 2.05 (m, 1H).

The following compounds are prepared in a similar manner as described above.

| Inter- | | HPLC RT | |
|---|---|---|---|
| mediate | Structure | (min) (method) | M + H |
| W1 | 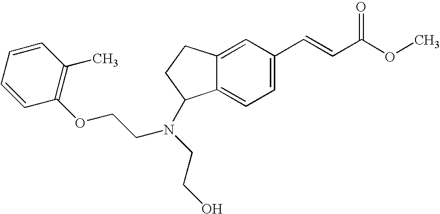 | 1.30 (A) | 396.0 |
| W2 | 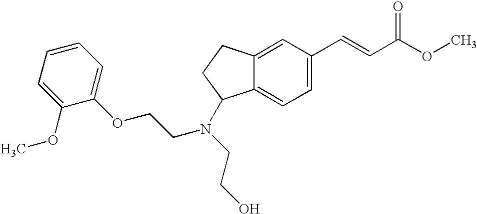 | 2.18 (A) | 412.0 |
| W3 | 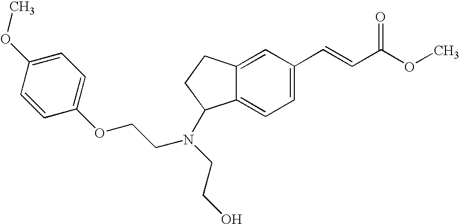 | 2.16 (A) | 412.0 |
| W4 | 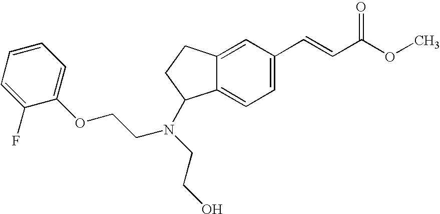 | 2.19 (A) | 399.9 |
| W5 | 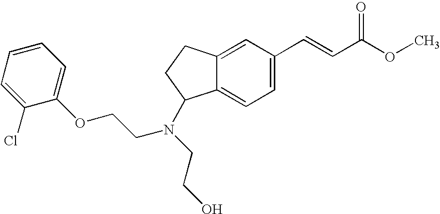 | 2.32 (A) | 416.0 |
| W6 | 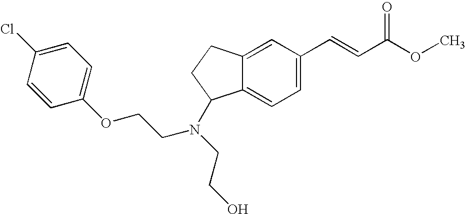 | 2.34 (A) | 416.0 |
| W7 | 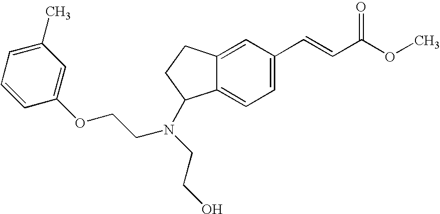 | 2.31 (A) | 396.0 |
| W8 | 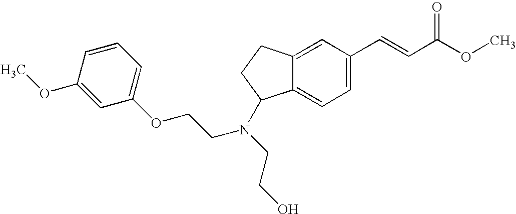 | 2.20 (A) | 412.0 |
| W9 | 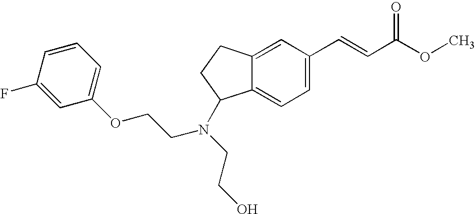 | 2.21 (A) | 399.9 |
| W10 | 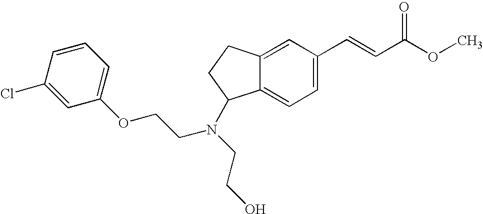 | 2.32 (A) | 416.0 |

### Intermediate X

### Methyl (2E)-3-(1-{(1H-indol-2-ylcarbonyl)[2-(1H-indol-2- yl)ethyl]amino}-2,3-dihydro-1H-inden-5-yl)acrylate

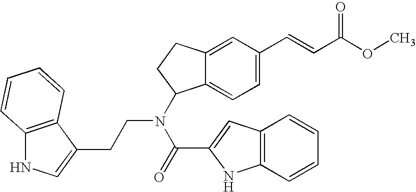

The HCl salt of intermediate E [Methyl (2E)-3-(1-{[2-(1H-indol-3-yl)ethyl]amino}-2,3-dihydro-1H-inden-5-yl)-2-propenoate hydrochloride] (0.15 g, 0.38 mmol) was dissolved in DMF (5 mL) and 2-indolecarboxylic acid (0.061 g, 0.34 mmol) was added followed by DIPEA (0.18 g, 1.37 mmol) and PyBOP (0.18 g, 0.34 mmol). The resulting mixture was stirred at room overnight, diluted with water and extracted with EtOAc. The organic layer was washed with 0.5 N HCl, water and brine and dried over Na₂SO4. The solvent was evaporated and the residue was purified by 40M Biotage eluting with hexane/EtOAc (6:1) to (3:1) to obtain Methyl (2E)-3-(1-{(1H-indol-2-ylcarbonyl)[2-(1H-indol-2- yl)ethyl]amino}-2,3-dihydro-1H-inden-5-yl)acrylate as a white solid (0.098 g, 57%): LC/MS [M+H] 504.0, RT 3.82 min (method A). ¹H-NMR (CD3OD) δ 7.70 (d, 1H), 7.60 (d, 1H), 7.52 (s, 1H), 7.46 (t, 2H), 7.26 (m, 3H), 7.06 (t, 1H), 7.01 (t, 1H), 6.88 (m, 3H), 6.53 (d, 1H), 6.17 (s, 1H), 3.57 (m, 1H), 2.99 (m, 6H), 2.43 (m, 1H), 2.05 (m, 1H).

The following compounds are prepared in a similar manner as described above.

| Inter- | | HPLC RT | |
|---|---|---|---|
| mediate | Structure | (min) (method) | M + H |
| X1 | 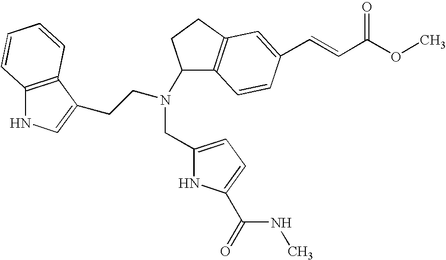 | 2.31 (A) | 497.0 |
| X2 | 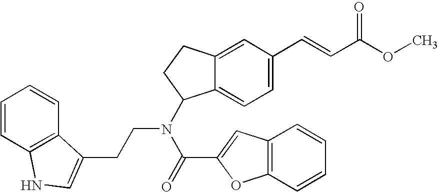 | 3.90 (A) | 505.0 |
| X3 | 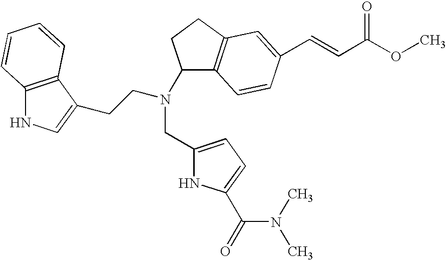 | 2.49 (A) | 511.0 |
| X4 | 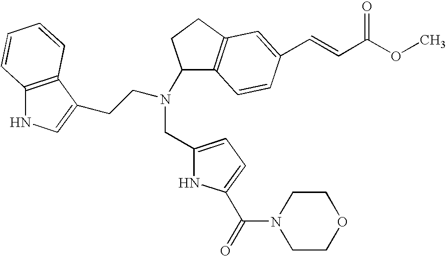 | 2.48 (A) | 553.0 |
| X5 | 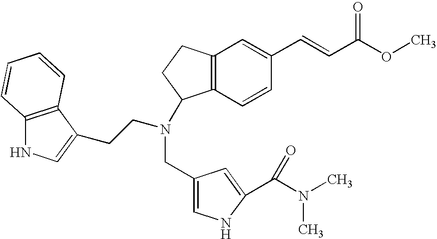 | 2.43 (A) | 511.1 |
| X6 | 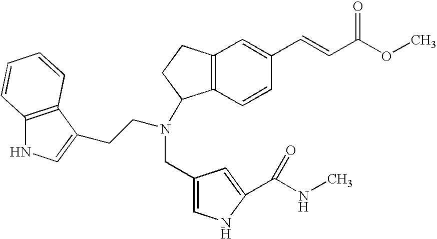 | 2.37 (A) | 497.1 |
| X7 | 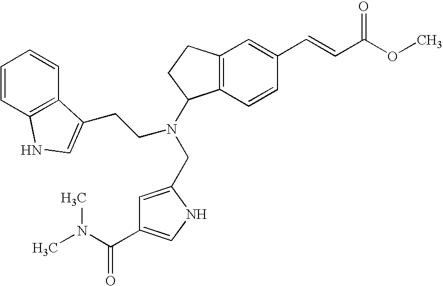 | 2.32 (A) | 511.0 |
| X8 | 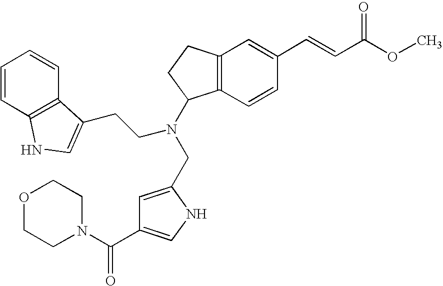 | 2.35 (A) | 553.0 |
| X9 | 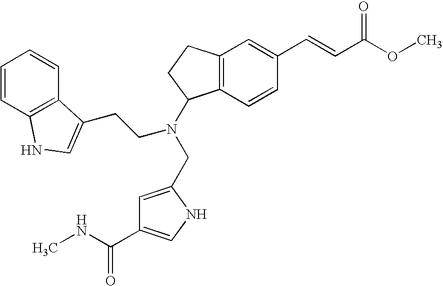 | 1.58 (A) | 497.0 |

### Intermediate Y

### Methyl (2E)-3-(1-}[(4-hydroxyphenyl)sulfonyl][2-(1H-indol-3-yl)ethyl]amino}-2,3-dihydro-1H-inden-5-yl)acrylate

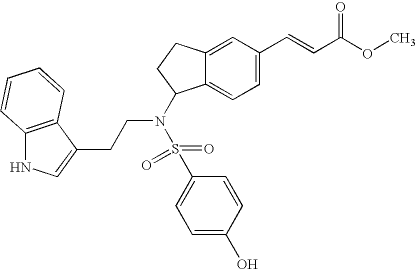

To a solution of intermediate O3 [methyl (2E)-3-(1-{{[4-(2-cyanoethoxy)phenyl]sulfonyl}[2-(1H-indol-3-yl)ethyl]amino}-2,3-dihydro-1H-inden-5-yl)acrylate] (0.30 g, 0.53 mmol) in MeOH (10 ml) was added K₂CO₃ (0.29 g, 2.1mmol). The mixture was stirred at rt for 5h under N₂. The reaction mixture was filtered to remove K₂CO₃. The filtrate was concentrated under vacuum to give a yellow residue. The residue was dissolved in CH₂Cl₂ and was washed with saturated NaHCO₃, brine and dried over Na₂SO₄. The solvent was evaporated to give methyl (2E)-3-(1-{[(4-hydroxyphenyl)sulfonyl][2-(1H-indol-3-yl)ethyl]amino}-2,3-dihydro-1H-inden-5-yl)acrylate as a dark oil (0.25 g, 91%). LC/MS [M+1]517.0, RT 3.26 min (Method A).

### Intermediate Z

### Methyl (2E)-3-(1-{{[4-(2-{[tert-butyl(dimethyl)silyl]oxy}ethoxy)phenyl]sulfonyl}[2(1H-indol-3-yl)ethyl]amino}-2.3-dihydro-1H-inden-5-yl)acrylate

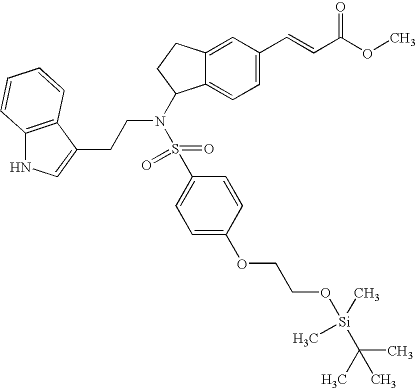

To a solution of intermediate Y [methyl (2E)-3-(1-{[(4-hydroxyphenyl)sulfonyl][2-(1H-indol-3-yl)ethyl]amino}-2,3-dihydro-1H-inden-5-yl)acrylate] (0.10 g, 0.17 mmol) in DMF (2 ml) was added (2-bromoethoxy)(tert-butyl)dimethyl silane (0.06 g, 0.26 mmol) and K₂CO₃ (0.096 g, 0.70 mmol). The mixture was stirred at 70 ° C. for 2 h. After cooled to rt, the mixture was diluted with EtOAc (20 ml) and washed with H₂O (3×10 mL), brine and dried over Na₂SO₄. The solvent was evaporated to give a 1:1 crude mixture of methyl (2E)-3-(1-{{[4-(2-{[tert-butyl(dimethyl)silyl]oxy}ethoxy)phenyl]sulfonyl}[2-(1H-indol-3-yl)ethyl]amino}-2,3-dihydro-1H-inden-5-yl)acrylate and 2-{[tert-butyl(dimethyl) silyl]oxy} ethyl (2E)-3-(1-{{[4-(2-{[tert -butyl(dimethyl)silyl]oxy} ethoxy) phenyl] sulfonyl}[2-1 H-1-indol-3-yl)ethyl]amino}-2,3-dihydro-1H-inden-5-yl)acrylate (110 mg). LC/MS [M+H] 675.0, RT 4.78 min and [M+H] 819.3, RT 5.66 min (method A).

The following compounds are prepared in a similar manner as described above.

| | | HPLC | |
|---|---|---|---|
| | | RT | |
| Inter- | | (min) | |
| mediate | Structure | (method) | M + H |
| Z1 | 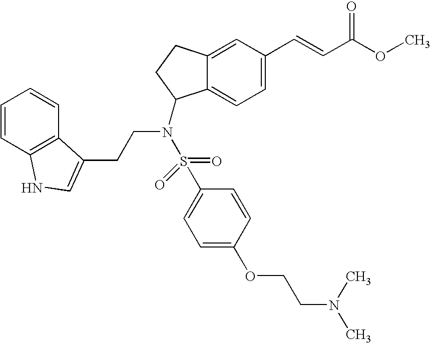 | 2.78 (A) | 588.1 |
| Z2 | 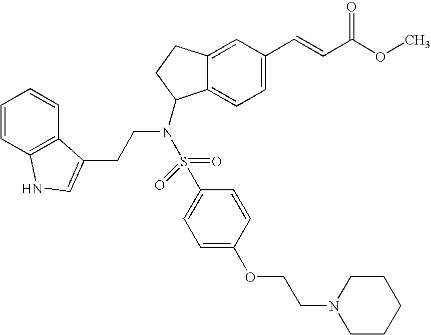 | 2.83 (A) | 628.4 |

### Intermediate AA

### Methyl (2E)-3-(1-{(2-{[(dimethylamino)sulfonyl]amino}ethyl)[2-(1H-indol-3-yl)ethyl]amino}-2,3-dihydro-1H-inden-5-yl)acrylate

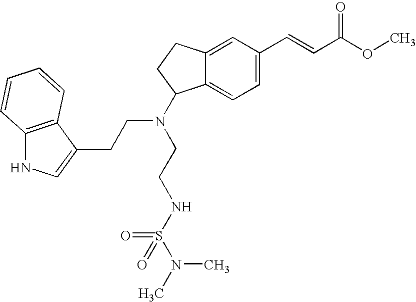

Intermediate T [methyl (2E)-3-(1-{(2-aminoethyl)[2-(1H-indol-3-yl)ethyl]amino}-2,3-dihydro-1H-inden-5-yl)acrylate] (150 mg, 0.37 mmol), and Et₃N (80 uL, 0.56 mmol) were dissolved in CH₂Cl₂ (3 mL). The solution was cooled to −40° C. and dimethylsulfamoyl chloride (50 uL, 0.41 mmol) was added. The reaction was warmed to rt and stirred for 18 h. The reaction was diluted with CH₂Cl₂ and washed with saturated NaHCO₃ solution and brine. The organic layer was collected, dried over anhydrous Na₂SO₄, filtered, followed by removal of solvent under vacuum. The crude product was purified by silica gel chromatography using 40% EtOAc/hexanes as eluent to obtain methyl (2E)-3-(1-{(2-{[(dimethylamino)sulfonyl]amino}ethyl)[2-(1H-indol-3-yl)ethyl]amino}-2,3-dihydro-1H-inden-5-yl)acrylate (123 mg, 65% yield) as an oily solid: LC/MS [M+H] 511.1, RT 2.36 min (method A).

### Intermediate AB

### Methyl (2E)-3-(1-{[(4-aminophenyl)sulfonyl][2-(1H-indol-3-yl)ethyl]amino}-2,3-dihydro-1H-inden-5-yl)acrylate

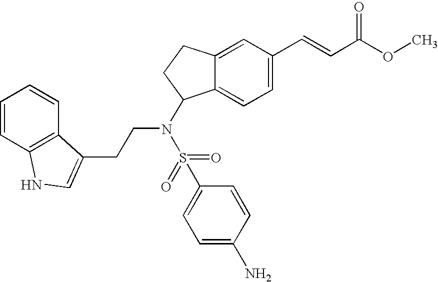

To a solution of intermediate O4 [methyl (2E)-3-(1-{{[4-(acetylamino)phenyl]sulfonyl}[2-(1H-indol-3-yl)ethyl]amino}-2,3-dihydro-1H-inden-5-yl)acrylate] (0.28g, 0.54mmol) in MeOH (10 ml) was slowly added 1M hydrogen chloride in 1,4-dioxane (0.8 ml). The mixture was heated to reflux for 5 h. After cooled to rt, the solvent was evaporated to give a yellow residue. The residue was purified on column chromatography with MeOH—CH₂Cl₂ (5/95, v/v) to give the desired product (0.26 g, 90%): LC/MS [M+H] 515.9, RT 3.48 min (method A).

### Intermediate AC

### Methyl (2E)-3-(1-{{{4-[(2-hydroxyethyl)amino]phenyl}sulfonyl)[2-(1H-indol-3-yl)ethyl]amino}-2,3-dihydro-1H-inden-5yl)acrylate

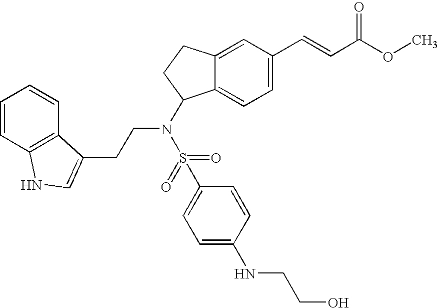

A solution of intermediate AB [methyl (2E)-3-(1-{[(4-aminophenyl)sulfonyl][2-(1H-indol-3-yl)ethyl]amino}-2,3-dihydro-1H-inden-5-yl)acrylate] (74 mg, 0.14 mmol) in MeOH (4 ml) was placed in a 20 ml sealed tube and was bubbled with ethylene oxide for 30 min. The sealed tube was closed and heated at 100° C. for 1 h. After cooled to rt, the crude was separated with reverse-phase preparative HPLC to give the desired compound (23 mg, 23%) with a 80% purity. No further purification was pursued. LC/MS [M+1] 559.9, RT 2.63 min (method A).

### Intermediate AD

### 1-Bromo-3-(chloromethyl)-2-fluorobenzene

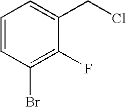

To a solution of 3-bromo-2-fluorophenyl)methanol (820 mg, 4.00 mmol) in toluene (10 mL was added thionyl chloride (0.44 mL, 6.00 mmol) and two drops of DMF. The reaction mixture was heated at 90° C. for 30 min, cooled to rt and concentrated down to afford intermediate 1-bromo-3-(chloromethyl)-2-fluorobenzene (890 mg, 99%). ¹H-NMR (DMSO-d6) δ 7.71 (m, 1H), 7.53 (m, 1H), 7.17 (m, 1H), 4.82 (d, 2H).

### Intermediate AE

### (3-Bromo-2-fluorophenyl)malonic acid

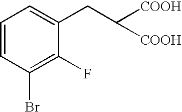

To a cold suspension of NaH (0.07 g, 3.10 mmol) in THF (5 mL) was added dropwise a solution of diethyl malonate (0.47 mL, 3.10 mmol) in THF (5 mL). The reaction was warmed up to rt and stirred for 1 h. Then a solution of intermediate AD [1-bromo-3-(chloromethyl)-2-fluorobenzene] (0.39 g, 1.72 mol) in THF (10 mL) was added dropwise. The reaction mixture was refluxed overnight, cooled and concentrated down. Water was added and extracted with CH₂Cl₂. The organic layer was washed with water, brine dried over Na₂SO₄, filtered and concentrated down. The residue was passed through a short silica gel pad to afford crude intermediate diethyl (3-bromo-2-fluorophenyl)malonate (0.59 g, 68%).

To a solution of diethyl (3-bromo-2-fluorophenyl)malonate (0.58 g, 1.73 mmol) in EtOH (20 mL) was added NaOH (50%, 2 mL). The reaction mixture was refluxed for 3 h, cooled down and concentrated. HCl (1N) was added to change pH to 4 and the mixture was extracted with ether. The organic layer was washed with water, brine, and dried over Na₂SO₄, filtered and concentrated down to afford (3-bromo-2-fluorophenyl)malonic acid as a white solid (0.45 g, 93%). ¹H-NMR (DMSO-d6) δ 12.91 (S, 2H), 7.54 (m, 1H), 7.30 (m, 1H), 7.06 (m, 1H), 3.57 (t, 1H), 3.07 (d, 2H).

### Intermediate AF

### (3-Bromo-2-fluorophenyl)acetic acid

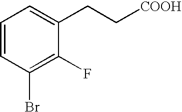

The solution of intermediate AE [3-bromo-2-fluorophenyl)malonic acid] (450 mg, 1.55 mmol) in dioxane (10 mL) was refluxed overnight, cooled down and concentrated. Water was added and the mixture was extracted with ether. The organic layer was washed with water, brine dried over Na₂SO₄, filtered and concentrated down afford (3-bromo-2-fluorophenyl)acetic acid as a white solid (370 mg, 96%). GC/MS [Exact Mass] 246; ¹H-NMR (DMSO-d6) δ 12.51 (S, 1H), 7.51 (m, 1H), 7.33 (m, 1H), 7.08 (m, 1H), 2.85 (t, 2H), 2.53 (t, 2H).

### Intermediate AG

### 5-Bromo-4-fluoroindan-1-one

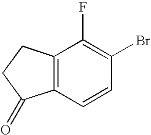

To the solution of intermediate AF [(3-bromo-2-fluorophenyl)acetic acid] (150 mg, 0.61 mmol) in CH₂Cl₂ (10 mL) was added thionyl chloride (0.13 mL, 1.82 mmol) and 2 drops of DMF. The mixture was stirred at rt overnight and concentrated down. The residue was dissolved in CH₂Cl₂ (5 mL) and then added to a cold solution of AlCl₃ in CH₂Cl₂ (5 mL). The reaction mixture was stirred at 0° C. for 20 min and then at rt for 3 h, poured into ice water and the mixture was extracted with CH₂Cl₂. The organic layer was washed with saturated NaHCO₃, brine, dried over Na₂SO₄, filtered and concentrated down. Chromatography using a Biotage cartridge (25S) with the EtOAc/Hexane (10/90) afforded 5-bromo4-fluoroindan-1-one (120 mg, 86%). GC/MS [Exact Mass] 228; ¹H-NMR (DMSO-d6) δ 7.74 (m, 1H), 7.40 (d, 1H), 3.12 (t, 2H), 2.68 (m, 2H).

### Intermediate AH

### N-(4-Formylphenyl)methanesulfonamide

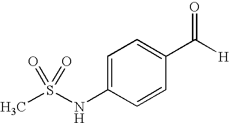

A solution of ethyl 4-aminobenzoate (1.00 g, 6.05 mmol) in pyridine (10 mL) was treated with methanesulfonyl chloride (1.11 g, 9.69 mmol) and stirred at rt for 1 hr. The mixture was diluted with EtOAc and H₂O and the layers were separated. The organic layer was washed with 1 N HCl, brine, and dried over MgSO₄. The solvent was removed at reduced pressure and the solid obtained was washed with Et₂O/hexanes to obtain Ethyl 4-[(methylsulfonyl)amino]benzoate as a light pink solid (1.29 g, 88%): ¹H NMR (DMSO-d6) δ 10.32 (s, 1H), 7.90 (d, 2H), 7.26 (d, 2H), 4.26 (q, 2H), 3.08 (s, 3H), 128 (t, 3H).

A LiAIH₄ solution (1.0 M in THF, 6.9 mL, 6.90 mmol) was added to an oven dried flask under nitrogen. The solution was diluted with THF (15 mL) and cooled to 0° C. A solution of ethyl 4-[(methylsulfonyl)amino]benzoate (1.29 g, 5.30 mmol) in THF (5 mL) was added dropwise to the LAH solution. After the addition, the reaction was warmed up to rt and stirred for 1 hr. TLC of a small aliquot showed complete reaction. The reaction was then cooled to 0° C. and treated carefully with EtOAc (5 mL), EtOH (5 mL), and 10% NaHSO₄ (7 mL). The suspension was filtered through celite and the filtrate was dried over MgSO₄. The solvent was removed at reduced pressure to obtain N-[4-(hydroxymethyl)phenyl]methanesulfonamide as a white solid (0.99 g, 93%): ¹H NMR (DMSO-d6) δ 9.62 (s, 1H), 7.24 (d, 2H), 7.13 (d, 2H), 5.12 (t, 1H), 4.42 (d, 2H), 2.92 (s, 3H).

N-[4-(hydroxymethyl)phenyl]methanesulfonamide (0.99 g, 4.91 mmol) was dissolved in THF (15 mL) and treated with MnO₂ (1.01 g, 9.83 mmol). The reaction was stirred at 50° C. overnight. The Manganese oxide was then filtered through celite and the filtrate was purified with 40 S Biotage eluting with 40-50% EtOAc in hexanes to obtain N-(4-formylphenyl)methanesulfonamide as a white solid (0.64 g, 65%): ¹H NMR (DMSO-d6) δ 10.47 (s, 1H), 9.86 (s, 1H), 7.86 (d, 2H), 7.33 (d, 2H), 3.13 (s, 3H).

By following the above procedures and those described for the amide formation (intermediate M, X), the sulfonamide formation (intermediate O), the urea formation (intermediate N), and the sulfonyl urea formation (intermediate AA), the following aldehydes are prepared in similar manners.

| Intermediate | Structure | TLC R_{f} (solvent) |
|---|---|---|
| AH1 | 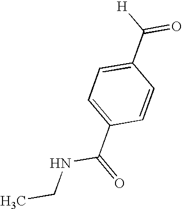 | 0.33 (EtOAc:hexanes, 1:1) |
| AH2 | 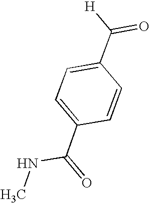 | 0.19 (EtOAc:hexanes, 1:1) |
| AH3 | 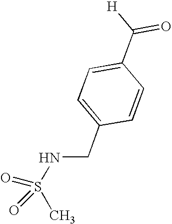 | 0.11 (EtOAc:hexanes, 2:3) |
| AH4 | 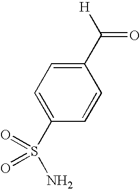 | 0.26 (EtOAc:CH₂Cl₂, 1:9) |
| AH5 | 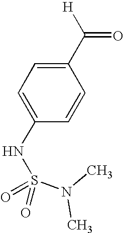 | 0.38 (EtOAc:hexanes, 2:3) |
| AH6 | 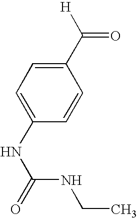 | 0.20 (EtOAc:hexanes, 2:3) |
| AH7 | 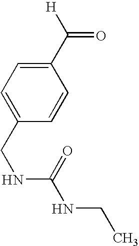 | 0.12 (EtOAc:hexanes, 3:2) |
| AH8 | 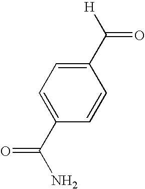 | 0.23 (EtOAc:hexanes, 3:2) |
| AH9 | 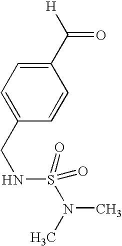 | 0.39 (EtOAc:hexanes, 2:3) |
| AH10 | 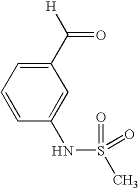 | 0.31 (EtOAc:hexanes, 2:3) |

### Compound Example 1

### (2E)-N-Hydroxy-3-((1R)-1-{[(1S)-2-hydroxy-1-(1H-indol-3-ylmethyl)ethyl]amino}-2,3-dihydro-1H-inden-5-yl)-2-propenamide

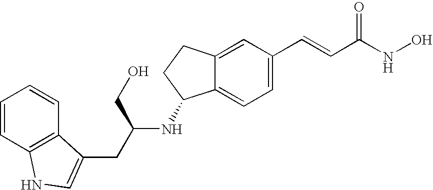

A mixture of Intermediate E1 (methyl (2E)-3-((1R)-1-{[(1S)-2-hydroxy-1-(1H-indol-3-ylmethyl)ethyl]amino}-2,3-dihydro-1H-inden-5-yl)-2-propenoate) (1.40 g, 3.59 mmol) and hydroxylamine hydrochloride (2.288 g, 32.27 mmol) in 40 mL MeOH was stirred for 10 min at rt, and cooled to ca. 5° C. with ice bath. KOH pellets (3.78 g, 57.3 mmol) was added to the cold reaction mixture. Ice bath was removed after 10 min. The reaction was allowed to warm to rt and was left stirring for 2 h. The reaction was quenched with 200 mL saturated aqueous NH₄Cl, extracted with EtOAc (5×200 mL). The combined extract was washed with saturated aqueous NaHCO₃, brine, and dried over Na₂SO₄. The solvent was removed under reduced pressure to yield (2E)-N-hydroxy-3-(1-{[(1S)-2-hydroxy-1-(1H-indol-3-ylmethyl)ethyl]amino-2,3-dihydro-1H-inden-5-yl)-2-propenamide (1.10 g, 78.%) as a white solid: LC/MS [M+H] 391.9, RT 1.65 min (method A); ¹H-NMR (DMSO-d6) δ 10.7(s, 1H), 10.6(broad, 1H), 9.0(broad, 1H), 7.51(d, 1H), 7.29-7.41(m, 5H), 7.13 (d, 1H), 7.05(t, 1H), 6.95 (t, 1H), 6.35 (d, 1H), 4.53 (d, 1H), 4.26 (t, 1H), 3.39 (s, 2H), 3.03 (m, 1H), 2.2.83-2.90 (m, 1H), 2.62-2.69 (m, 4H), 2.24-2.29 (m, 1H) and 1.35-1.41 (m, 1H).

### Compound Example 193

### (−)-(2E)-N-hydroxy-3-(1-(2-hydroxyethyl)[2-(1H-indol-3-yl)ethyl]amino}-2,3-dihydro-1H-inden-5-yl)but-2-enamide

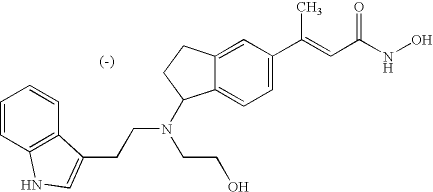 Intermediate R3 [methyl (2E)-3-{(1S)-1-[(2-hydroxyethyl)amino]-2,3-dihydro-1H-inden-5-yl)but-2-enoate -3-propyl-1H-indole] (0.445 g, 1.06 mmol) was dissolved in dioxane (10 mL) and cooled to 0° C. NH₂OH (10 mL, 50% in water) was added to above mixture followed by 1N NaOH (10 mL). The resulting solution was stirred at 0° C. for 2 h and at rt for another hour. The reaction was quenched with NH₄Cl saturated solution, diluted with EtOAc (30 mL) and stirred until both layers become clear. The organic layer was separated, washed with brine, dried over Na₂SO4 and concentrated to obtain the desired product (0.31 g, 70%): LC/MS [M+H] 420.2, RT 1.83 min (method A). ¹H-NMR (CD₃OD) δ 7.26 (m, 5H), 7.14 (m, 2H), 6.90 (t, 1H), 6.02 (s, 1H), 4.71 (s, 1H), 3.63 (m, 2H), 2.92 (m, 8H), 2.51 (s, 3H), 2.33 (m, 1H), 2.10 (m, 1H).

With the exception of compound example 43, 44, 45, 46, 181, 182, and 183, all other compound examples in table 1 are synthesized in a similar manner as described above for compound example 1 and 193.

### Compound Example 43

### (2E)-N-Hydroxy-3-(2-{[2-(1H-indol-3-yl)ethyl]amino}-2,3-dihydro-1H-inden-5-yl)-2-propenamide

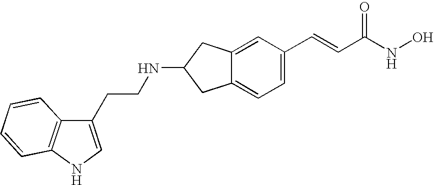

Compound Example 10 (tert-Butyl 3-[2-(tert-butoxycarbonyl){5-[(1E)-3-(hydroxyamino)-3-oxo-1-propenyl]-2,3-dihydro-1H-inden-2-yl)amino)ethyl]-1H-indole-1-carboxylate) (119 mg, 0.21 mmol) was dissolved in CH₂Cl₂ (4 mL) and TFA (1 mL) was added. The solution was stirred for 1 h before the solvent was removed under vacuum. The crude material was dissolved in a small amount of EtOAc. This solution was added to a stirring solution of 1:1 NaHCO₃/Na₂CO₃ (pH 9). The product precipitated out and the mixture was filtered. The solid was collected and dried to give (2E)-N-hydroxy-3-(2-{([2-(1H-indol-3-yl)ethyl]amino}-2,3-dihydro-1H-inden-5-yl)-2-propenamide as a white solid (46 mg, 60%): ¹H-NMR (DMSO-d6) δ 10.82 (s, 1H), 7.52 (d, 1H), 7.17-7.42 (m, 6H), 6.95-7.08 (m, 2H), 6.38 (d, 1H), 3.74 (m, 1H), 2.77-3.17 (m, 8H).

Examples 44, 45, and 46 are synthesized in a similar manner. In the case of examples 45 and 46, 95% TFA in water is used.

### Compound Example 182

### N-{(1R)-5-[(1E)-3-(Hydroxyamino)-3-oxoprop-1-en-1-yl]-2,3-dihydro-1H-inden-1-yl}-N-[(1S)-2-hydroxy-1-(1H-indol-3-ylmethyl)ethyl]benzamide

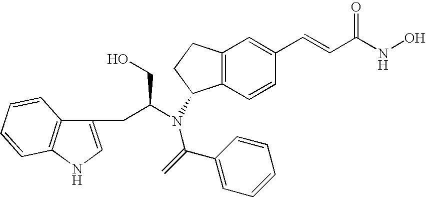

A mixture of intermediate E1 [methyl (2E)-3-((1R)-1-{[(1S)-2-hydroxy-1-(1H-indol-3-ylmethyl)ethyl]amino}-2,3-dihydro-1H-inden-5-yl)acrylate] (0.2 g, 0.51 mmol), benzoyl chloride (0.28 g, 2.05 mmol) and Et₃N (0.29 ml, 2.05 mmol) in CH₂Cl₂ (2 ml) was stirred at rt for 16h. To the crude mixture was added MeOH (2 ml ) and hydroxylamine hydrochloride salt (0.32 g, 4.61 mmol). The reaction mixture was stirred at rt for 10 min. and was cooled to 0° C. with an ice-water bath. Potassium hydroxide (0.67 g, 10.24 mmol) was added to the reaction mixture as pellets. The reaction was continued to stir for 1 h. The reaction was quenched with saturated aqueous NH₄Cl, and extracted with EtOAc. The combined extract was washed with saturated aqueous NaHCO₃, brine, and dried over Na₂SO₄. The solvent was removed and the crude product was purified with reverse-phase preparative HPLC to give the desired product as an oil (4 mg, 1.5%). LC/MS [M+1] 496.0, RT 2.45 min (Method A).

Examples 181 and 183 are prepared in a similar manner as described above.

Pro-drugs of this invention in general may be made by conventional methods well known in the art. For example, the hydroxyl groups may be converted to esters by reacting the compounds with carboxylic acid chlorides or anhydrides under standard conditions. The hydroxyl groups may also be converted to carbonates by reacting the compounds with chloroformates under standard conditions.

Salts of the compounds identified herein can be obtained by isolating the compounds as hydrochloride salts, prepared by treatment of the free base with anhydrous HCl in a suitable solvent such as THF. Generally, a desired salt of a compound of this invention can be prepared in situ during the final isolation and purification of a compound by means well known in the art. Or, a desired salt can be prepared by separately reacting the purified compound in its free base form with a suitable organic or inorganic acid and isolating the salt thus formed. These methods are conventional and would be readily apparent to one skilled in the art.

Additionally, sensitive or reactive groups on the compound of this invention may need to be protected and deprotected during any of the above methods. Protecting groups in general may be added and removed by conventional methods well known in the art (see, for example, T. W. Greene and P. G. M. Wuts, Protective Groups in Organic Synthesis; Wiley: New York, (1999).

Compositions of the Compounds of this Invention

The compounds of this invention can be utilized to achieve the desired pharmacological effect by administration to a patient in need thereof in an appropriately formulated pharmaceutical composition. The present invention includes pharmaceutical compositions that are comprised of a pharmaceutically acceptable carrier and a pharmaceutically effective amount of a compound, or salt thereof, of the present invention. A pharmaceutically acceptable carrier is any carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, otically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable soluhon or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations which are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients which can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CC₂F₂, F₂ClC—CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate); antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono-or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

It is believed that one skilled in the art, using the preceding information, can utilize the present invention to its fullest extent. Nevertheless, the following are examples of pharmaceutical formulations that can be used in the composition of the present invention. They are for illustrative purposes only, and are not to be construed as limiting the invention in any way.

Pharmaceutical compositions according to the present invention can be illustrated as follows:
Sterile IV Solution: A 2 mg/mL solution of the desired compound of this invention is made using sterile, injectable water, and the pH is adjusted if necessary. The solution is diluted for administration to 0.2-1 mg/mL with sterile 5% dextrose and is administered as an IV infusion over 120 minutes.
Lyophilized Dowder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-120 min.
Intramuscular suspension: The following solution or suspension can be prepared, for intramuscular injection:

50 mg/mL of the desired, water-insoluble compound of this invention

5 mg/mL sodium carboxymethylcellulose

4 mg/mL TWEEN 80

9 mg/mL sodium chloride

9 mg/mL benzyl alcohol
Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.
Soft Gelatin CaDsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.
Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.
Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.
   Method of Treating Hyper-Proliferative Disorders

Another embodiment of the present invention relates to a method of using the compounds described above, including salts and pro-drugs thereof and corresponding compositions thereof, to treat mammalian hyper-proliferative disorders. This method comprises administering to a patient an amount of a compound of this invention, or a pharmaceutically acceptable salt thereof, which is effective to treat the patient's hyper-proliferative disorder. A patient, for the purpose of this invention, is a mammal, including a human, in need of treatment for a particular hyper-proliferative disorder. Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The utility of the compounds of the present invention can be illustrated, for example, by their activity in vitro in the in vitro tumor cell proliferation assay described below. The link between activity in tumor cell proliferation assays in vitro and anti-tumor activity in the clinical setting has been very well established in the art. For example, the therapeutic utility of taxol (Silvestrini et al. Stem Cells 1993, 11(6), 528-35), taxotere (Bissery et al. Anti Cancer Drugs 1995, 6(3), 339), and topoisomerase inhibitors (Edelman et al. Cancer Chemother. Pharmacol. 1996, 37(5), 385-93) was demonstrated with the use of in vitro tumor proliferation assays.

The following assay is one of the methods by which compound activity relating to treatment of the disorders identified herein can be determined.

In vitro Tumor Cell Proliferation Assay

The adherent tumor cell proliferation assay used to test the compounds of the present invention involves a readout called Cell Titre-Glo developed by Promega (Cunningham, B A “A Growing Issue: Cell Proliferation Assays. Modern kits ease quantification of cell growth” The Scientist 2001, 15(13), 26, and Crouch, S P et al., “The use of ATP bioluminescence as a measure of cell proliferation and cytotoxicity” Journal of Immunological Methods 1993, 160, 81-88).

HCT116 cells (colon carcinoma, purchased from ATCC) or A549 (lung carcinoma, purchased from ATCC) were plated in 96-well plates at 3000 cells/well in complete media with 10% Fetal Calf Serum and incubated 24 h at 37° C. Twenty-four h after plating, test compounds were added over a final concentration range of 10 nM to 20 μM in serial dilutions at a final DMSO concentration of 0.2%. Cells were incubated for 72 h at 37° C. in complete growth media after addition of the test compound. On day 4, using a Promega Cell Titer Glo Luminescent® assay kit, the cells are lysed and 100 microliters of substrate/buffer mixture is added to each well, mixed and incubated at room temperature for 8 min. The samples were read on a luminometer to measure the amount of ATP present in the cell lysates from each well, which corresponds to the number of viable cells in that well. Values read at 24 h incubation were subtracted as Day 0. For determination of IC50's, a linear regression analysis were used to determine drug concentration which results in a 50% inhibition of cell proliferation using this assay format.

Representative compounds of this invention showed a significant inhibition of tumor cell proliferation in the assays with HCT116 cells (>50% inhibition at 10 uM) and representative compounds were also studied with the A549 cells and found to be active. MDA-MB-231 (breast adenocarcinoma, purchased from ATCC), LnCaP (prostate carcinoma, purchased from ATCC), H460 (lung carcinoma, purchased from ATCC), or Hela (cervix adenocarcinoma) cells can also be used in similar assays.

Based upon the above and other standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.01 mg/kg to about 200 mg/kg, and preferably from about 0.1 mg/kg to about 20 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or composition thereof can be ascertained by those skilled in the art using conventional treatment tests.

The compounds of this invention can be administered as the sole pharmaceutical agent or in combination with one or more other pharmaceutical agents where the combination causes no unacceptable adverse effects. For example, the compounds of this invention can be combined with known anti-hyper-proliferative or other indication agents, and the like, as well as with admixtures and combinations thereof.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with this invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′, 2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine. Other anti-hyper-proliferative agents suitable for use with this invention include but are not limited to other anti-cancer agents such as epothilone, irinotecan, raloxifen and topotecan.

It is believed that one skilled in the art, using the preceding information, can utilize the present invention to its fullest extent.

It should be apparent to one of ordinary skill in the art that changes and modifications can be made to this invention without departing from the spirit or scope of the invention as it is set forth herein.

What is claimed is:

1. A compound of Formula I 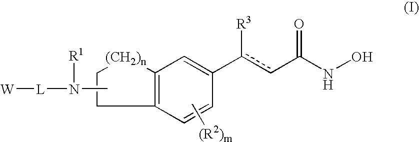
wherein
W is selected from H, (C₁-C₆)alkyl,
O-phenyl optionally substituted with up to 2 substituents each selected independently from R¹²,
phenyl optionally substituted with up to 2 substituents each selected independently from R¹², OH, COOR⁷, C(O)NHR⁷, S(O)₂(C₁-C₃)alkyl, NHS(O)₂(C₁-C₃)alkyl, N[(C₁-C₃)alkyl]₂, NH(C₁-C₃)alkyl,
NHC(O)(C₁-C₃)alkyl, 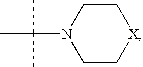
and
(C₁-C₃)alkoxy substituted with 1 substituent selected from N[(C₁-C₃)alkyl]₂, NH(C₁-C₃)alkyl, and 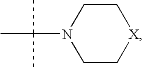
indolyl optionally substituted with 1 or 2 substituents each selected independently from R¹², OH, C(O)O(C₁-C₄)alkyl,
(C₁-C₃)alkyl substituted with 1 or 2 substituents each selected independently from OH, C(O)R⁸, (C₁-C₃)alkoxy, pyrrolidinyl, 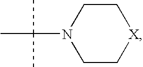
imidazolyl, NH(C₁-C₃)alkyl, and N[(C₁-C₃)alkyl]₂, and
(C₁-C₃)alkoxy substituted with 1 substituent selected from NH(C₁-C₃)alkyl, N[(C₁-C₃)alkyl]₂, pyrrolidinyl, imidazolyl, 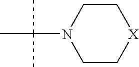
and (C₁-C₃)alkoxy, and
another heteroaryl optionally substituted with up to 3 substituents each independently selected from R¹²;
L is selected from CHR⁴, CHR⁵—CHR⁶, and CHR⁵—CH₂-CHR⁶;
R¹ is selected from H, C(O)R¹⁰, C(O)OR⁷, tetrahydropyranyl, (C₃-C₆)cycloalkyl, phenyl optionally substituted with up to 2 substituents each independently selected from R¹²,
pyridyl, optionally substituted with up to 2 substituents each independently selected from R¹²
S(O)₂-phenyl where said phenyl is optionally substituted with 1 or 2 substituents each independently selected from R¹²,NH₂, NHC(O)(C₁-C₃)alkyl, NH(C₁-C₃)alkyl-N[(C₁-C₃)alkyl]₂ NH(C₁-C₃)alkyl-OH, COOH, OH, and (C₁-C₃)alkoxy substituted with 1 substituent selected from N[(C₁-C₃)alkyl]₂, OH, and 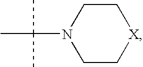
S(O)₂(C₁-C₃)alkyl optionally substituted with one phenyl ring,
(C₁-C₆)alkyl optionally substituted with 1 or 2 substituents each independently selected from OR¹¹, C(O)R¹⁰, C(O)OR⁷, N[(C₁-C₃)alkyl]₂,
(C₃-C₆)cycloalkyl, dioxopyrrolidinyl, 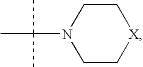
glucopyranosyl, glucopyranosylamino,
(C₁-C₃)alkoxy optionally substituted with 1 or 2 substituents each selected independently from OH, 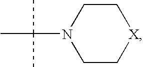
and imidazolyl,
O-phenyl optionally substituted with up to two substituents each independently selected from R¹²,
NH₂ where one H is optionally replaced with one substituent selected from S(O)₂(C₁-C₃)alkyl, S(O)₂NH(C₁-C₃)alkyl, S(O)₂CF₃, C(O)R⁷, S(O)₂N[(C₁-C₃)alkyl]₂, C(O)O(C₁-C₄)alkyl, C(O)NH(C₁-C₄)alkyl, C(O)N[(C₁-C₃)alkyl]₂, 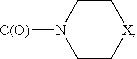
and (C₁-C₄)alkyl optionally substituted with one OH group,
phenyl optionally substituted with 1 or 2 substituents each independently selected from R¹², OH, S—(C₁-C₃)alkyl, C(O)NH₂, S(O)₂NH₂, C(O)N[(C₁-C₃)alkyl]₂, S(O)₂(C₁-C₃)alkyl, S(O)₂NHC(O)(C₁-C₃)alkyl, C(O)(C₁-C₃)alkyl, C(O)NH(C₁-C₃)alkyl, NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHC(O)NH₂, S(O)₂N[(C₁-C₃)alkyl]₂, NHS(O)₂NH(C₁-C₃)alkyl, NHC(O)(C₁-C₃)alkyl, S(O)₂NH(C₁-C₃)alkyl optionally substituted with 1 substituent selected from (C₁-C₃)alkoxy, NH(C₁-C₃)alkyl, N[(C₁-C₃)alkyl]₂, and 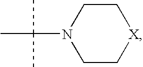
(C₁-C₃)alkyl substituted with one substituent selected from NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHS(O)₂NH(C₁-C₃)alkyl, and NHC(O)(C₁-C₃)alkyl, and
(C₁-C₃)alkoxy substituted with 1 substituent selected from OH, NH(C₁-C₃)alkyl, N[(C₁-C₃)alkyl]₂, (C₁-C₃)alkoxy, and 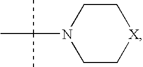
pyrrolyl optionally substituted with one substituent selected from R¹², C(O)N[(C₁-C₃)alkyl]₂, C(O)NH(C₁-C₃)alkyl, C(O)(C₁-C₃)alkyl, 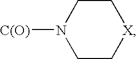
and S(O)₂(C₁-C₃)alkyl,
pyrazolyl optionally substituted with up to 3 substituents each selected independently from R¹², C(O)N[(C₁-C₃)alkyl]₂, C(O)NH(C₁-C₃)alkyl, and 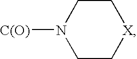
and
another heteroaryl optionally substituted with up to two substituents each independently selected from R¹²;
R² is in each instance selected independently from (C₁-C₃)alkyl, halo, (C₁-C₃)alkoxy, CF₃, NO₂, NH₂, CN, and COOH;
R³ is selected from H, (C₁-C₃)alkyl, and halo;
R⁴ is selected from H and (C₁-C₃)alkyl-OH;
R⁵ is selected from H, OH and (C₁-C₃)alkyl;
R⁶ is selected from H, C(O)OR⁷, C(O)R⁹, and
(C₁-C₆)alkyl optionally substituted with one substituent selected from OH, NHS(O)₂(C₁-C₃)alkyl, and NHC(O)(C₁-C₃)alkyl;
R⁷ is selected from H and (C₁-C₄)alkyl;
R⁸ is selected from OH, NH₂, N[(C₁-C₃)alkyl]₂, morpholinyl, and pyrrolidinyl;
R⁹ is selected from NH₂, morpholinyl, N[(C₁-C₃)alkyl]₂, and
NH(C₁-C₃)alkyl optionally substituted with one substituent selected from OH, COOH, and N[(C₁-C₃)alkyl]₂;
R¹⁰ is selected from (C₃-C₆)cycloalkyl, morpholinyl, N[(C₁-C₄)alkyl]₂, (C₁-C₃)alkoxy, heteroaryl optionally substituted with 1 or 2 substituents each independently selected from (C₁-C₃)alkyl, (C₁-C₃)alkoxy, OH, halo and CF₃,
phenyl optionally substituted with 1 or 2 substituents each independently selected from (C₁-C₃)alkyl, (C₁-C₃)alkoxy, OH, halo and CF₃,
(C₁-C₃)alkyl optionally substituted with one substituent selected from phenyl, imidazolyl, and 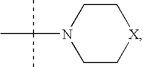
N
H(C₁-C₄)alkyl optionally substituted with 1 phenyl ring optionally substituted with 1 or 2 substituents each independently selected from (C₁-C₃)alkyl, (C₁-C₃)alkoxy, halo and CF₃, and
NH-phenyl where said phenyl is optionally substituted with 1 or 2 substituents each independently selected from (C₁-C₃)alkyl, (C₁-C₃)alkoxy, halo and CF₃;
R¹¹ is selected from H, C(O)N[(C₁-C₃)alkyl]₂, C(O)-pyrrolidinyl, C(O)NH-phenyl, and C(O)NH(C₁-C₃)alkyl optionally substituted with 1 phenyl ring;
R¹² is selected from (C₁-C₆)alkyl, (C₁-C₃)alkoxy, halo, NO₂, CN, CF₃, O—CF₃, and
phenyl optionally substituted with up to 2 substituents each selected independently from halo, (C₁-C₃)alkyl, and (C₁-C₃)alkoxy;
X is selected from O, S, CH₂, and NH, and
when X is NH, the H on NH is optionally replaced with C(O)(C₁-C₃)alkyl, S(O)₂(C₁-C₃)alkyl, or (C₁-C₆)alkyl
and when X is O, S, or CH₂, the 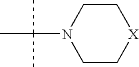
moiety is optionally substituted by replacing any H atom in the 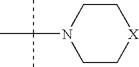
moiety with (C₁-C₄)alkyl;
m is selected from 0, 1 and 2;
n is selected from 1 and 2; [symbol] is selected from a double bond and a single bond;
or a pharmaceutically acceptable salt, ester or carbonate thereof.

2. A compound of claim 1 wherein
R¹ is selected from H, C(O)R¹⁰, tetrahydropyranyl, (C₃-C₆)cycloalkyl,
S(O)₂-phenyl where said phenyl is optionally substituted with 1 or 2 substituents each independently selected from R¹²,—NH₂, NHC(O)(C₁-C₃)alkyl, NH(C₁-C₃)alkyl-N[(C₁-C₃)alkyl]₂, NH(C₁-C₃)alkyl-OH, COOH, OH, and (C₁-C₃)alkoxy substituted with 1 substituent selected from N[(C₁-C₃)alkyl]₂, OH, 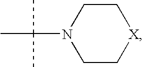
and
S(O)₂(C₁-C₃)alkyl optionally substituted with one phenyl ring,
(C₁-C₆)alkyl optionally substituted with 1 or 2 substituents each independently selected from OR¹¹, C(O)R¹⁰, C(O)OR⁷, N[(C₁-C₃)alkyl]₂,
(C₃-C₆)cycloalkyl, dioxopyrrolidinyl, 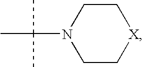
(
C₁-C₃)alkoxy optionally substituted with 1 or 2 substituents each selected independently from OH, 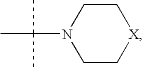
and imidazolyl,
O-phenyl optionally substituted with up to two substituents each independently selected from R¹²,
NH₂ where one H is optionally replaced with one substituent selected from S(O)₂(C₁-C₃)alkyl, S(O)₂NH(C₁-C₃)alkyl, S(O)₂CF₃, C(O)R⁷, S(O)₂N[(C₁-C₃)alkyl]₂, C(O)NH(C₁-C₄)alkyl, C(O)N[(C₁-C₃)alkyl]₂, 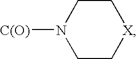
and (C₁-C₄)alkyl optionally substituted with one OH group,
phenyl optionally substituted with 1 or 2 substituents each independently selected from R¹², OH, S—(C₁-C₃)alkyl, C(O)NH₂, S(O)₂NH₂, C(O)N[(C₁-C₃)alkyl]₂, S(O)₂(C₁-C₃)alkyl, S(O)₂NHC(O)(C₁-C₃)alkyl, C(O)(C₁-C₃)alkyl, C(O)NH(C₁-C₃)alkyl, NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHC(O)NH₂, S(O)₂N[(C₁-C₃)alkyl]₂, NHS(O)₂NH(C₁-C₃)alkyl, NHC(O)(C₁-C₃)alkyl, S(O)₂NH(C₁-C₃)alkyl optionally substituted with 1 substituent selected from (C₁-C₃)alkoxy, NH(C₁-C₃)alkyl,
N[(C₁-C₃)alkyl]₂, and 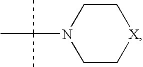
(C₁-C₃)alkyl substituted with one substituent selected from NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHS(O)₂NH(C₁-C₃)alkyl, and NHC(O)(C₁-C₃)alkyl, and
(C₁-C₃)alkoxy substituted with 1 substituent selected from OH, NH(C₁-C₃)alkyl, N[(C₁-C₃)alkyl]₂, (C₁-C₃)alkoxy, and 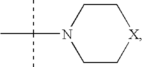
pyrrolyl optionally substituted with one substituent selected from R¹²,
C(O)N[(C₁-C₃)alkyl]₂, C(O)NH(C₁-C₃)alkyl, C(O)(C₁-C₃)alkyl, 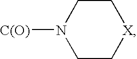
and S(O)₂(C₁-C₃)alkyl,
pyrazolyl optionally substituted with up to 3 substituents each selected independently from R¹², C(O)N[(C₁-C₃)alkyl]₂, C(O)NH(C₁-C₃)alkyl, and 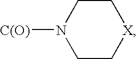
another heteroaryl optionally substituted with up to two substituents each independently selected from R¹²;
R¹⁰ is selected from (C₃-C₆)cycloalkyl, N[(C₁-C₄)alkyl]₂, (C₁-C₃)alkyl, NH(C₁-C₄)alkyl,
heteroaryl optionally substituted with 1 or 2 substituents each independently selected from (C₁-C₃)alkyl, (C₁-C₃)alkoxy, OH, halo and CF₃,
phenyl optionally substituted with 1 or 2 substituents each independently selected from (C₁-C₃)alkyl, (C₁-C₃)alkoxy, OH, halo and CF₃;
R¹¹ is H;
R¹² is selected from (C₁-C₃)alkyl, (C-C₃)alkoxy, halo, NO₂, CN, CF₃, and O—CF₃; and
m is selected from 0 and 1.

3. A compound of claim 1 wherein
W is selected from
O-phenyl optionally substituted with up to 2 substituents selected from R¹²,
phenyl optionally substituted with up to 2 substituents each selected independently from R¹², OH, COOR⁷, C(O)NHR⁷, S(O)₂(C₁-C₃)alkyl, NHS(O)₂(C₁-C₃)alkyl, N[(C₁-C₃)alkyl]₂, NH(C₁-C₃)alkyl, NHC(O)(C₁-C₃)alkyl, 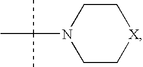
and
(C₁-C₃)alkoxy substituted with 1 substituent selected from N[(C₁-C₃)alkyl]₂, NH(C₁-C₃)alkyl, and 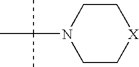
indolyl optionally substituted with 1 or 2 substituents each selected independently from R¹², OH, C(O)O(C₁-C₄)alkyl,
(C₁-C₃)alkyl substituted with 1 or 2 substituents each selected independently from OH, C(O)R⁸, (C₁-C₃)alkoxy, pyrrolidinyl, 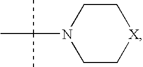
imidazolyl, NH(C₁-C₃)alkyl and N[(C₁-C₃)alkyl]₂, and
(C₁-C₃)alkoxy substituted with 1 substituent selected from NH(C₁-C₃)alkyl, N[(C₁-C₃)alkyl]₂, pyrrolidinyl, imidazolyl, 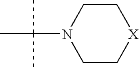
and (C₁-C₃)alkoxy, and
another heteroaryl optionally substituted with up to 3 substituents each independently selected from R¹²;
R¹ is selected from H, C(O)R¹⁰, tetrahydropyranyl, (C₃-C₆)cycloalkyl,
S(O)₂-phenyl where said phenyl is optionally substituted with 1 or 2 substituents each independently selected from R¹²,—NH₂, NHC(O)(C₁-C₃)alkyl, NH(C₁-C₃)alkyl-N[(C₁-C₃)alkyl]₂ NH(C₁-C₃)alkyl-OH, COOH, OH, and (C₁-C₃)alkoxy substituted with 1 substituent selected from N[(C₁-C₃)alkyl]₂, OH, and 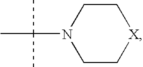
S
(O)₂(C₁-C₃)alkyl optionally substituted with one phenyl ring,
(C₁-C₆)alkyl optionally substituted with 1 or 2 substituents each independently selected from OR¹¹, C(O)R¹⁰, C(O)OR⁷, N[(C₁-C₃)alkyl]₂,
(C₃-C₆)cycloalkyl, dioxopyrrolidinyl, 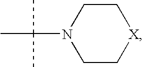
(C₁-C₃)alkoxy optionally substituted with 1 or 2 substituents each selected independently from OH, 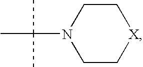
and imidazolyl,
O-phenyl optionally substituted with up to two substituents each independently selected from R¹²,
NH₂ where one H is optionally replaced with one substituent selected from S(O)₂(C₁-C₃)alkyl, S(O)₂NH(C₁-C₃)alkyl, S(O)₂CF₃, C(O)R⁷, S(O)₂N[(C₁-C₃)alkyl]₂, C(O)NH(C₁-C₄)alkyl, C(O)N[(C₁-C₃)alkyl]₂, 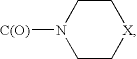
and (C₁-C₄)alkyl optionally substituted with one OH group,
phenyl optionally substituted with 1 or 2 substituents each independently selected from R¹², OH, S—(C₁-C₃)alkyl, C(O)NH₂, S(O)₂NH₂, C(O)N[(C₁-C₃)alkyl]₂, S(O)₂(C₁-C₃)alkyl, S(O)₂NHC(O)(C₁-C₃)alkyl, C(O)(C₁-C₃)alkyl, C(O)NH(C₁-C₃)alkyl, NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHC(O)NH₂, S(O)₂N[(C₁-C₃)alkyl]₂, NHS(O)₂NH(C₁-C₃)alkyl, NHC(O)(C₁-C₃)alkyl, S(O)₂NH(C₁-C₃)alkyl optionally substituted with 1 substituent selected from (C₁-C₃)alkoxy, NH(C₁-C₃)alkyl, N[(C₁-C₃)alkyl]₂, and 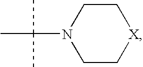
(C₁-C₃)alkyl substituted with one substituent selected from NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHS(O)₂NH(C₁-C₃)alkyl, and NHC(O)(C₁-C₃)alkyl, and
(C₁-C₃)alkoxy substituted with 1 substituent selected from OH, NH(C₁-C₃)alkyl, N[(C₁-C₃)alkyl]₂, (C₁-C₃)alkoxy, and 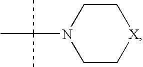
pyrrolyl optionally substituted with one substituent selected from R¹², C(O)N[(C₁-C₃)alkyl]₂, C(O)NH(C₁-C₃)alkyl, C(O)(C₁-C₃)alkyl, 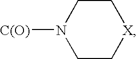
and S(O)₂(C₁-C₃)alkyl,
pyrazolyl optionally substituted with up to 3 substituents each selected independently from R¹², C(O)N[(C₁-C₃)alkyl]₂, C(O)NH(C₁-C₃)alkyl, and 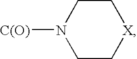
another heteroaryl optionally substituted with up to two substituents each independently selected from R¹²;
R² is in each instance selected independently from (C₁-C₃)alkyl, halo, (C₁-C₃)alkoxy or CF₃;
R⁴ and R⁵ are each H;
R⁶ is selected from H, and
(C₁-C₆)alkyl optionally substituted with one substituent selected from OH, NHS(O)₂(C₁-C₃)alkyl, and NHC(O)(C₁-C₃)alkyl;
R¹⁰ is selected from (C₃-C₆)cycloalkyl, N[(C₁-C₄)alkyl]₂, (C₁-C₃)alkyl, NH(C₁-C₄)alkyl, heteroaryl optionally substituted with 1 or 2 substituents each independently selected from (C₁-C₃)alkyl, (C₁-C₃)alkoxy, OH, halo and CF₃,
phenyl optionally substituted with 1 or 2 substituents each independently selected from (C₁-C₃)alkyl, (C₁-C₃)alkoxy, OH, halo and CF₃;
R¹¹ is H;
R¹² is selected from (C₁-C₃)alkyl, (C₁-C₃)alkoxy, halo, NO₂, CN, CF₃, and O—CF₃; and
m is selected from 0 and 1.

4. A compound of claim 3 wherein
W is selected from
phenyl optionally substituted with up to 2 substituents each selected independently from R¹², and
(C₁-C₃)alkoxy substituted with 1 substituent selected from N[(C₁-C₃)alkyl]₂, NH(C₁-C₃)alkyl, and 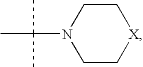
indolyl optionally substituted with 1 or 2 substituents each selected independently from R¹²,
(C₁-C₃)alkyl substituted with 1 or 2 substituents each selected independently from OH and (C₁-C₃)alkoxy, and
another heteroaryl optionally substituted with up to 3 substituents each independently selected from R¹².

5. A compound of claim 3 wherein L is CHR⁵—CHR⁶.

6. A compound of claim 3 wherein
R¹ is selected from H, C(O)R¹⁰, tetrahydropyranyl,
S(O)₂-phenyl where said phenyl is optionally substituted with 1 or 2 substituents each independently selected from R¹², COOH, OH, and
(C₁-C₃)alkoxy substituted with 1 substituent selected from N[(C₁-C₃)alkyl]₂, OH, and 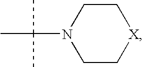
(C₁-C₆)alkyl optionally substituted with 1 or 2 substituents each independently selected from OR¹¹, N[(C₁-C₃)alkyl]₂,
(C₃-C₆)cycloalkyl, 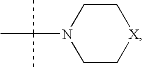
(C₁-C₃)alkoxy optionally substituted with 1 or 2 OH,
NH₂ where one H is replaced with one substituent selected from S(O)₂(C₁-C₃)alkyl, S(ONH(C₁-C₃)alkyl, S(O)₂CF₃, C(O)R⁷, S(O)₂N[(C₁-C₃)alkyl]₂, C(O)NH(C₁-C₄)alkyl, C(O)N[(C₁-C₃)alkyl]₂, 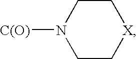
and (C₁-C₄)alkyl optionally substituted with one OH group,
phenyl optionally substituted with 1 or 2 substituents each independently selected from R¹², OH, C(O)NH₂, S(O)₂NH₂, S(O)₂NHC(O)(C₁-C₃)alkyl, C(O)NH(C₁-C₃)alkyl, NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHC(O)NH₂, NHS(O)₂NH(C₁-C₃)alkyl, NHC(O)(C₁-C₃)alkyl, S(O)₂NH(C₁-C₃)alkyl optionally substituted with 1 substituent selected from (C₁-C₃)alkoxy, NH(C₁-C₃)alkyl, N[(C₁-C₃)alkyl]₂, and 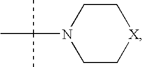
(C₁-C₃)alkyl substituted with one substituent selected from NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHS(O)₂NH(C₁-C₃)alkyl, and NHC(O)(C₁-C₃)alkyl, and
(C₁-C₃)alkoxy substituted with 1 substituent selected from OH, NH(C₁-C₃)alkyl, N[(C₁-C₃)alkyl]₂, (C₁-C₃)alkoxy, and 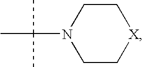
pyrrolyl optionally substituted with one substituent selected from R¹², C(O)N[(C₁-C₃)alkyl]₂, C(O)NH(C₁-C₃)alkyl, C(O)(C₁-C₃)alkyl, 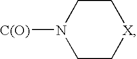
pyrazolyl optionally substituted with up to 3 substituents each selected independently from R¹², C(O)N[(C₁-C₃)alkyl]₂, C(O)NH(C₁-C₃)alkyl, and 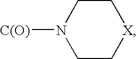
another heteroaryl optionally substituted with up to two substituents each independently selected from R¹².

7. A compound of claim 1 wherein
W is selected from
phenyl optionally substituted with up to 2 substituents each selected independently from R¹², and
(C₁-C₃)alkoxy substituted with 1 substituent selected from N[(C₁-C₃)alkyl]₂, NH(C₁-C₃)alkyl, and 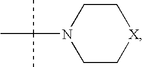
indolyl optionally substituted with 1 or 2 substituents each selected independently from R¹²,
(C₁-C₃)alkyl substituted with 1 or 2 substituents each selected independently from OH, (C₁-C₃)alkoxy, 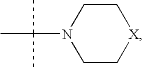
another heteroaryl optionally substituted with up to 3 substituents each independently selected from R¹²;
L is CHR⁵—CHR⁶;
R¹ is selected from H, C(O)R¹⁰, tetrahydropyranyl,
S(O)₂-phenyl where said phenyl is optionally substituted with 1 or 2 substituents each independently selected from R¹², COOH, OH, and (C₁-C₃)alkoxy substituted with 1 substituent selected from N[(C₁-C₃)alkyl]₂, OH, and 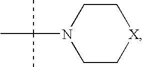
(C₁-C₆)alkyl optionally substituted with 1 or 2 substituents each independently selected from OR¹¹, N[(C₁-C₃)alkyl]₂,
(C₃-C₆)cycloalkyl, 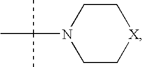
(C₁-C₃)alkoxy optionally substituted with 1 or 2 OH groups,
NH₂ where one H is replaced with one substituent selected from S(O)₂(C₁-C₃)alkyl, S(O)₂NH(C₁-C₃)alkyl, S(O)₂CF₃, C(O)R⁷, S(O)₂N[(C₁-C₃)alkyl]₂, C(O)NH(C₁-C₄)alkyl, C(O)N[(C₁-C₃)alkyl]₂, 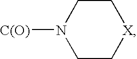
and (C₁-C₄)alkyl optionally substituted with one OH group,
phenyl optionally substituted with 1 or 2 substituents each independently selected from R¹², OH, C(O)NH₂, S(O)₂NH₂, S(O)₂NHC(O)(C₁-C₃)alkyl, C(O)NH(C₁-C₃)alkyl, NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHC(O)NH₂, NHS(O)₂NH(C₁-C₃)alkyl, NHC(O)(C₁-C₃)alkyl, S(O)₂NH(C₁-C₃)alkyl optionally substituted with 1 substituent selected from (C₁-C₃)alkoxy, NH(C₁-C₃)alkyl, N[(C₁-C₃)alkyl]₂, and 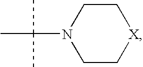
(C₁-C₃)alkyl substituted with one substituent selected from NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHS(O)₂NH(C₁-C₃)alkyl, and NHC(O)(C₁-C₃)alkyl, and
(C₁-C₃)alkoxy substituted with 1 substituent selected from OH, NH(C₁-C₃)alkyl, N[(C₁-C₃)alkyl]₂, (C₁-C₃)alkoxy, and 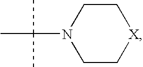
pyrrolyl optionally substituted with one substituent selected from R¹², C(O)N[(C₁-C₃)alkyl]₂, C(O)NH(C₁-C₃)alkyl, C(O)(C₁-C₃)alkyl, 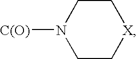
and S(O)₂(C₁-C₃)alkyl,
pyrazolyl optionally substituted with up to 3 substituents each selected independently from R¹², C(O)N[(C₁-C₃)alkyl]₂, C(O)NH(C₁-C₃)alkyl, and 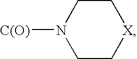
and
another heteroaryl optionally substituted with up to two substituents each independently selected from R¹²;
R² is halo;
R⁵ is H;
R⁶ is selected from H, and
(C₁-C₆)alkyl optionally substituted with one substituent selected from OH, NHS(O)₂(C₁-C₃)alkyl, and NHC(O)(C₁-C₃)alkyl;
R¹⁰ is selected from (C₃-C₆)cycloalkyl, N[(C₁-C₄)alkyl]₂, (C₁-C₃)alkyl and NH(C₁-C₄)alkyl-,
R¹¹ is H;
R¹² is selected from (C₁-C₃)alkyl, (C₁-C₃)alkoxy, halo, NO₂, CN, CF₃, and O—CF₃; and
m is selected from 0, and 1.

8. A compound of claim 7 wherein
W is selected from
phenyl optionally substituted with up to 2 substituents each selected independently from R¹², and
(C₁-C₃)alkoxy substituted with 1 substituent selected from N[(C₁-C₃)alkyl]₂, NH(C₁-C₃)alkyl, and 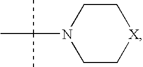
indolyl optionally substituted with 1 or 2 substituents each selected independently from R¹²
(C₁-C₃)alkyl substituted with 1 or 2 substituents each selected independently from OH, and (C₁-C₃)alkoxy.

9. A compound of claim 7 wherein
R¹ is selected from H,
S(O)₂-phenyl where said phenyl is optionally substituted with 1 or 2 substituents each independently selected from R¹², COOH, and OH,
(C₁-C₆)alkyl optionally substituted with 1 or 2 substituents each independently selected from OR¹¹, (C₃-C₆)cycloalkyl, 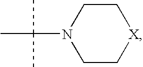
and (C₁-C₃)alkoxy optionally substituted with 1 or 2 OH groups,
NH₂ where one H is replaced with one substituent selected from S(O)₂(C₁-C₃)alkyl, S(O)₂NH(C₁-C₃)alkyl, S(O)₂CF₃, C(O)R⁷, S(O)₂N[(C₁-C₃)alkyl]₂, C(O)NH(C₁-C₄)alkyl, C(O)N[(C₁-C₃)alkyl]₂, 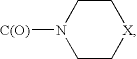
and (C₁-C₄)alkyl optionally substituted with one OH group,
phenyl optionally substituted with 1 or 2 substituents each independently selected from R¹², OH, C(O)NH₂, S(O)₂NH₂, S(O)₂NHC(O)(C₁-C₃)alkyl, C(O)NH(C₁-C₃)alkyl, NHS(O)₂(C₁-C₃)alkyl, NHS(O)2N[(C₁-C₃)alkyl]₂, NHC(O)NH(C₁-C₃)alkyl, NHC(O)N[(C₁-C₃)alkyl]₂, NHC(O)NH₂, NHS(O)₂NH(C₁-C₃)alkyl, NHC(O)(C₁-C₃)alkyl, S(O)₂NH(C₁-C₃)alkyl optionally substituted with 1 substituent selected from (C₁-C₃)alkoxy, NH(C₁-C₃)alkyl, N[(C₁-C₃)alkyl]₂, and 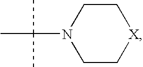
(C₁-C₃)alkyl substituted with one substituent selected from NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHS(O)₂NH(C₁-C₃)alkyl, and NHC(O)(C₁-C₃)alkyl, and
(C₁-C₃)alkoxy substituted with 1 substituent selected from OH, NH(C₁-C₃)alkyl, N[(C₁-C₃)alkyl]₂, (C₁-C₃)alkoxy, and 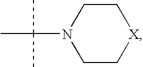
pyrrolyl optionally substituted with one substituent selected from R¹², C(O)N[(C₁-C₃)alkyl]₂, C(O)NH(C₁-C₃)alkyl, C(O)(C₁-C₃)alkyl, and 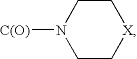
pyrazolyl optionally substituted with up to 3 substituents each selected independently from R¹², C(O)N[(C₁-C₃)alkyl]₂, C(O)NH(C₁-C₃)alkyl, and

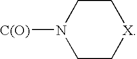

10. A compound of claim 1 wherein
W is selected from
phenyl optionally substituted with up to 2 substituents each selected independently from R¹², and
(C₁-C₃)alkoxy substituted with 1 substituent selected from N[(C₁-C₃)alkyl]₂, NH(C₁-C₃)alkyl, and 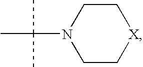
indolyl optionally substituted with 1 or 2 substituents each selected independently from R¹²,
(C₁-C₃)alkyl substituted with 1 or 2 substituents each selected independently from OH, and (C₁-C₃)alkoxy;
L is CHR⁵—CHR⁶;
R¹is selected from H,
S(O)₂-phenyl where said phenyl is optionally substituted with 1 or 2 substituents each independently selected from R¹², COOH, and OH,
(C₁-C₆)alkyl optionally substituted with 1 or 2 substituents each independently selected from OR¹¹, (C₃-C₆)cycloalkyl, 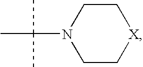
and (C₁-C₃)alkoxy optionally substituted with 1 or 2 OH groups,
NH₂ where one H is replaced with one substituent selected from S(O)₂(C₁-C₃)alkyl, S(O)₂NH(C₁-C₃)alkyl, S(0)₂CF₃, C(O)R⁷, S(O)₂N[(C₁-C₃)alkyl]₂, C(O)NH(C₁-C₄)alkyl, C(O)N[(C₁-C₃)alkyl]₂, 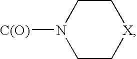
and (C₁-C₄)alkyl optionally substituted with one OH group,
phenyl optionally substituted with 1 or 2 substituents each independently selected from R¹², OH, C(O)NH₂, S(O)₂NH₂, S(O)₂N HC(O)(C₁-C₃)alkyl, C(O)NH(C₁-C₃)alkyl, NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHC(O)NH₂, NHS(O)₂NH(C₁-C₃)alkyl, NHC(O)(C₁-C₃)alkyl, S(O)₂NH(C₁-C₃)alkyl optionally substituted with 1 substituent selected from (C₁-C₃)alkoxy, NH(C₁-C₃)alkyl, N[(C₁-C₃)alkyl]₂, and 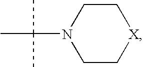
(C₁-C₃)alkyl substituted with one substituent selected from NHS(O)₂(C₁-C₃)alkyl, NHS(O)₂N[(C₁-C₃)alkyl]₂, NHC(O)NH(C₁-C₃)alkyl, NHC(O)N[(C₁-C₃)alkyl]₂, NHS(O)₂NH(C₁-C₃)alkyl, and NHC(O)(C₁-C₃)alkyl, and
(C₁-C₃)alkoxy substituted with 1 substituent selected from OH, NH(C₁-C₃)alkyl, N[(C₁-C₃)alkyl]₂, (C₁-C₃)alkoxy, and 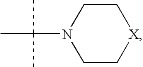
pyrrolyl optionally substituted with one substituent selected from R¹², C(O)N[(C₁-C₃)alkyl]₂, C(O)NH(C₁-C₃)alkyl, C(O)(C₁-C₃)alkyl, and 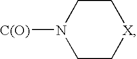
pyrazolyl optionally substituted with up to 3 substituents each selected independently from R¹², C(O)N[(C₁-C₃)alkyl]₂, C(O)NH(C₁-C₃)alkyl, and 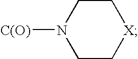
R² is halo;
R³ is selected from H, and (C₁)alkyl;
R⁵ is H;
R⁶ is selected from H, and (C₁-C₆)alkyl optionally substituted with one OH group;
R⁷ is selected from H and (C₁-C₄)alkyl;
R¹¹ is H;
R¹² is selected from (C₁-C₃)alkyl, (C₁-C₃)alkoxy, halo, CN, and CF₃;
m is selected from 0, and 1; and
n is 1.

11. A compound of claim 10 wherein L is CH₂—CH₂

12. A pharmaceutical composition comprising a compound of claim 1.

13. A pharmaceutical composition comprising a compound of claim 2.

14. A pharmaceutical composition comprising a compound of claim 3.

15. A pharmaceutical composition comprising a compound of claim 4.

16. A pharmaceutical composition comprising a compound of claim 5.

17. A pharmaceutical composition comprising a compound of claim 6.

18. A pharmaceutical composition comprising a compound of claim 7.

19. A pharmaceutical composition comprising a compound of claim 8.

20. A pharmaceutical composition comprising a compound of claim 9.

21. A pharmaceutical composition comprising a compound of claim 10.

22. A pharmaceutical composition comprising a compound of claim 11.

23. A method of treating a hyper-proliferative disorder in a mammal comprising administering to a mammal in need thereof a pharmaceutically effective amount of a compound of claim 1.

24. A method of treating a hyper-proliferative disorder in a mammal comprising administering to a mammal in need thereof a pharmaceutically effective amount of a compound of claim 2.

25. A method of treating a hyper-proliferative disorder in a mammal comprising administering to a mammal in need thereof a pharmaceutically effective amount of a compound of claim 3.

26. A method of treating a hyper-proliferative disorder in a mammal comprising administering to a mammal in need thereof a pharmaceutically effective amount of a compound of claim 4.

27. A method of treating a hyper-proliferative disorder in a mammal comprising administering to a mammal in need thereof a pharmaceutically effective amount of a compound of claim 5.

28. A method of treating a hyper-proliferative disorder in a mammal comprising administering to a mammal in need thereof a pharmaceutically effective amount of a compound of claim 6.

29. A method of treating a hyper-proliferative disorder in a mammal comprising administering to a mammal in need thereof a pharmaceutically effective amount of a compound of claim 7.

30. A method of treating a hyper-proliferative disorder in a mammal comprising administering to a mammal in need thereof a pharmaceutically effective amount of a compound of claim 8.

31. A method of treating a hyper-proliferative disorder in a mammal comprising administering to a mammal in need thereof a pharmaceutically effective amount of a compound of claim 9.

32. A method of treating a hyper-proliferative disorder in a mammal comprising administering to a mammal in need thereof a pharmaceutically effective amount of a compound of claim 10.

33. A method of treating a hyper-proliferative disorder in a mammal comprising administering to a mammal in need thereof a pharmaceutically effective amount of a compound of claim 11.

34. A method of any of claim 23, claim 24, claim 25, claim 26, claim 27, claim 28, claim 29, claim 30, claim 31, claim 32, and claim 33, wherein the hyper-proliferative disorder is selected from solid tumors, lymphomas, sarcomas and leukemias.

35. A method of claim 34 wherein the disorder is selected from solid tumors.

36. A method according to claim 35 wherein the tumor is selected from cancers of the breast, reproductive organs, respiratory tract, brain, head, neck, hematopoietic tissue, digestive tract and urinary tract.

37. A method according to claim 36 wherein the disorder is selected from cancers of the breast, prostate, ovary, lung, colon, head, neck and hematopoietic tissue.
